(19) United States
(12) Patent Application Publication
(10) Pub. No.: US 2021/0355503 A1
(43) Pub. Date: Nov. 18, 2021

(54) COMPOSITIONS AND METHODS FOR MANUFACTURING GENE THERAPY VECTORS

(71) Applicant: NightstaRx Limited, Maidenhead, Berkshire (GB)

(72) Inventors: Julian Hanak, London (GB); Richard Truran, London (GB)

(21) Appl. No.: 17/277,877

(22) PCT Filed: Sep. 23, 2019
(86) PCT No.: PCT/US2019/052501

§ 371 Date: Mar. 19, 2021

Related U.S. Application Data
(60) Provisional application No. 62/734,505, filed on Sep. 21, 2018.

(51) Int. Cl. C12N 15/86; B01D 15/32; B01D 15/36; B01D 15/42; B01D 69/08

(52) U.S. Cl. CPC C12N 15/86; B01D 15/327; B01D 15/362; B01D 15/363; C12N 2750/14123; B01D 69/08; C12N 2750/14151; C12N 2750/14152; B01D 15/424

(57) ABSTRACT

Disclosed are methods for the production and/or purification of a recombinant AAV (rAAV) particle from a mammalian host cell culture.

### CROSS REFERENCE TO RELATED APPLICATIONS

This application claims priority to U.S. Provisional Application No. 62/734,505, filed on Sep. 21, 2018; which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 23, 2019, is named NIGH-015/001WO_SL.txt and is 308 kilobytes in size.

### FIELD OF THE DISCLOSURE

The disclosure relates to the fields of human therapeutics, biologic drug products, viral delivery of human DNA sequences and methods of manufacturing same.

### BACKGROUND

There is a long-felt and unmet need for AAV-based delivery vectors and improved methods of manufacturing these AAV-based delivery vectors.

### SUMMARY

The disclosure provides a method of purifying a recombinant AAV (rAAV) particle from a mammalian host cell culture, comprising the steps of: (a) purifying the plurality of rAAV particles through hydrophobic interaction chromatography (HIC) to produce a HIC eluate comprising the plurality of rAAV particles; (b) purifying the HIC eluate of (a) through cation exchange chromatography (CEX) to produce a CEX eluate comprising a plurality of rAAV particles; (c) isolating a plurality of full rAAV particles from the CEX eluate of (b) by anion exchange (AEX) chromatography to produce a AEX eluate comprising a purified and enriched plurality of full rAAV particles; and (d) diafiltering and concentrating the AEX eluate from (c) into a formulation buffer by tangential flow filtration (TFF) to produce a final composition comprising a purified and enriched plurality of full rAAV particles and the final formulation buffer. In some embodiments, the method further comprises the steps of contacting a plurality of transfected mammalian host cells and a virus release solution under conditions suitable for the release of the plurality of rAAV particles into a harvest media to produce a composition comprising a plurality of rAAV particles, virus release solution and harvest media; and purifying the plurality of rAAV particles from the composition through hydrophobic interaction chromatography (HIC) to produce a HIC eluate comprising the plurality of rAAV particles. In some embodiments, the method further comprises the step of culturing a plurality of mammalian host cells in a harvest media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells, prior to the contacting step. In some embodiments, the AAV is an AAV8 or a derivative thereof. In some embodiments, the AAV comprises an AAV8 capsid protein or a derivative thereof.

In some embodiments of the methods of the disclosure, the harvest media comprises one or more of Dulbecco's Modified Eagle's medium (DMEM), stabilized glutamine, stabilized glutamine dipeptide and Benzonase.

In some embodiments of the methods of the disclosure, the harvest media comprises glycine, L-Arginine hydrochloride, L-Cystine dihydrocholoride, L-Glutamine, L-Histidine hydrochloride-H2O, L-Isoleucine, L-Leucine, L-Lysine hydrochloride, L-Methionine, L-Phenylalanine, L-Serine, L-Threonine, L-Tryptophan, L-Tyrosine disodium salt dehydrate, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl2) (anhyd.), Ferric Nitrate (Fe(NO3)3″9H2O), Magnesium Sulfate (MgSO4) (anhyd.), Potassium Chloride (KCl), Sodium Bicarbonate (NaHCO₃), Sodium Chloride (NaCl), Sodium Phosphate monobasic (NaH2PO4-H2O), and D-Glucose (Dextrose).

In some embodiments of the methods of the disclosure, the harvest media comprises 4 mM stabilized glutamine or stabilized glutamine dipeptide.

In some embodiments of the methods of the disclosure, the harvest media comprises a serum-free media. In some embodiments of the methods of the disclosure, the harvest media consists of a serum-free media.

In some embodiments of the methods of the disclosure, the harvest media comprises a protein-free media. In some embodiments of the methods of the disclosure, the harvest media consists of a protein-free media.

In some embodiments of the methods of the disclosure, the harvest media comprises a clarified media. In some embodiments of the methods of the disclosure, the harvest media consists of a clarified media.

In some embodiments of the methods of the disclosure, the exogenous sequence comprises: (a) a sequence encoding a rhodopsin kinase promoter; (b) a sequence encoding a retinitis pigmentosa GTPase regulator ORF15 isoform (RPGR^{ORF15}); and (c) a sequence encoding a polyadenylation (polyA) signal.

In some embodiments of the methods of the disclosure, the rhodopsin kinase promoter is a GRK1 promoter. In some embodiments, wherein the sequence encoding the GRK1 promoter comprises or consists of:

| (SEQ ID NO: 5) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments of the methods of the disclosure, the sequence encoding the RPGR^{ORF15} is a codon optimized human RPGR^{ORF15} sequence. In some embodiments, the sequence encoding RPGR^{ORF15} comprises a nucleotide sequence encoding an amino acid sequence of:

| (SEQ ID NO: 78) | |
|---|---|
| 1 | MREPEELMPD SGAVFTFGKS KFAENNPGKF WFKNDVPVHL SCGDEHSAVV TGNNKLYMFG |
| 61 | SNNWGQLGLG SKSAISKPTC VKALKPEKVK LAACGRNHTL VSTEGGNVYA TGGNNEGQLG |
| 121 | LGDTEERNTF HVISFFTSEH KIKQLSAGSN TSAALTEDGR LFMWGDNSEG QIGLKNVSNV |
| 181 | CVPQQVTIGK PVSWISCGYY HSAFVTTDGE LYVFGEPENG KLGLPNQLLG NHRTPQLVSE |
| 241 | IPEKVIQVAC GGEHTVVLTE NAVYTFGLGQ FGQLGLGTFL FETSEPKVIE NIRDQTISYI |
| 301 | SCGENHTALI TDIGLMYTFG DGRHGKLGLG LENFTNHFIP TLCSNFLRFI VKLVACGGCH |
| 361 | MVVFAAPHRG VAKEIEFDEI NDTCLSVATF LPYSSLTSGN VLQRTLSARM RRRERERSPD |
| 421 | SFSMRRTLPP IEGTLGLSAC FLPNSVFPRC SERNLQESVL SEQDLMQPEE PDYLLDEMTK |
| 481 | EAEIDNSSTV ESLGETTDIL NMTHIMSLNS NEKSLKLSPV QKQKKQQTIG ELTQDTALTE |
| 541 | NDDSDEYEEM SEMKEGKACK QHVSQGIFMT QPATTIEAFS DEEVEIPEEK EGAEDSKGNG |
| 601 | IEEQEVEANE ENVKVHGGRK EKTEILSDDL TDKAEVSEGK AKSVGEAEDG PEGRGDGTCE |
| 661 | EGSSGAEHWQ DEEREKGEKD KGRGEMERPG EGEKELAEKE EWKKRDGEEQ EQKEREQGHQ |
| 721 | KERNQEMEEG GEEEHGEGEE EEGDREEEEE KEGEGKEEGE GEEVEGEREK EEGERKKEER |
| 781 | AGKEEKGEEE GDQGEGEEEE TEGRGEEKEE GGEVEGGEVE EGKGEREEEE EEGEGEEEEG |
| 841 | EGEEEEGEGE EEEGEGKGEE EGEEGEGEEE GEEGEGEGEE EEGEGEGEEE GEGEGEEEEG |
| 901 | EGEGEEEGEG EGEEEEGEGK GEEEGEEGEG EGEEEEGEGE GEDGEGEGEE EEGEWEGEEE |
| 961 | EGEGEGEEEG EGEGEEGEGE GEEEEGEGEG EEEEGEEEGE EEGEGEEEGE GEGEEEEEGE |
| 1021 | VEGEVEGEEG EGEGEEEEGE EEGEEREKEG EGEENRRNRE EEEEEEGKYQ ETGEEENERQ |
| 1081 | DGEEYKKVSK IKGSVKYGKH KTYQKKSVTN TQGNGKEQRS KMPVQSKRLL KNGPSGSKKF |
| 1141 | WNNVLPHYLE LK. |

In some embodiments, the sequence encoding RPGR^{ORF15} comprises or consists of a nucleotide sequence of:

| (SEQ ID NO: 80) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3061 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa. |

In some embodiments of the methods of the disclosure, the sequence encoding the polyA signal comprises a bovine growth hormone (BGH) polyA sequence. In some embodiments, the sequence encoding the BGH polyA signal comprises a nucleotide sequence of:

| (SEQ ID NO: 83) | |
|---|---|
| 1 | cgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg. |

In some embodiments of the methods of the disclosure, the exogenous sequence comprises a sequence encoding an ATP Binding Cassette, Subfamily Member 4 (ABCA4) protein or a portion thereof. In some embodiments, the exogenous sequence comprises a 5′ sequence encoding an ABCA4 protein or a portion thereof. In some embodiments, the exogenous sequence comprises a 3′ sequence encoding an ABCA4 protein or a portion thereof.

In some embodiments of the methods of the disclosure, the exogenous sequence further comprises a sequence encoding a promoter. In some embodiments, the exogenous sequence further comprises a sequence encoding a rhodopsin kinase (RK) promoter. In some embodiments, the RK promoter is a GRK1 promoter.

In some embodiments of the methods of the disclosure, the sequence encoding the GRK1 promoter comprises or consists of:

| (SEQ ID NO: 5) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments of the methods of the disclosure, the exogenous sequence further comprises a sequence encoding a chicken beta-actin (CBA) promoter. In some embodiments, the sequence encoding the CBA promoter comprises or consists of:

| (SEQ ID NO: 16) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |
| or | |
| (SEQ ID NO: 24) | |
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

In some embodiments of the methods of the disclosure, the sequence encoding the ABCA4 is a human ABCA4 sequence. In some embodiments, the sequence encoding ABCA4 comprises a 5′ nucleotide sequence comprising nucleotides 1-4500 of SEQ ID NO: 2 or SEQ ID NO: 1, or a 3′ truncation variant thereof of either. In some embodiments, the sequence encoding ABCA4 comprises a 5′ nucleotide sequence comprising nucleotides 1-3701 or 1-4326 of SEQ ID NO: 2 or SEQ ID NO: 1. In some embodiments, the sequence encoding ABCA4 comprises a 3′ nucleotide sequence comprising nucleotides 3000-6822 of SEQ ID NO: 2 or SEQ ID NO: 1, or a 5′ truncation variant thereof of either. In some embodiments, the sequence encoding ABCA4 comprises a 3′ nucleotide sequence comprising nucleotides 3154-6822, 3196-6822, 3494-6822, 3603-6822, 3653-6822, 3678-6822, 3702-6822 or 3494-6822 of SEQ ID NO:2 or SEQ ID NO: 1. In some embodiments, the sequence encoding ABCA4 comprises a 5′ nucleotide sequence comprising nucleotides 1-4326 of SEQ ID NO: 2 or SEQ ID NO: 1 and the sequence encoding ABCA4 comprises a 3′ nucleotide sequence comprising nucleotides 3154-6822 of SEQ ID NO: 2 or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3196-6822 of SEQ ID NO: 2. or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3494-6822 of SEQ ID NO:2 or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3603-6822 of SEQ ID NO:2 or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3653-6822 of SEQ ID NO:2 or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3678-6822 of SEQ ID NO:2 or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3702-6822 of SEQ ID NO:2 or SEQ ID NO: 1. In some embodiments, the sequence encoding ABCA4 comprises a 5′ nucleotide sequence comprising nucleotides 1-3701 and the sequence encoding ABCA4 comprises a 3′ nucleotide sequence comprising nucleotides 3494-6822 of SEQ ID NO:2 or SEQ ID NO: 1.

SEQ ID NO: 1 is the human ABCA4 nucleic acid sequence corresponding to NCBI Reference Sequence NM_000350.2. SEQ ID NO: 1 is identical to NCBI Reference Sequence NM_000350.2. The ABCA4 coding sequence spans nucleotides 105 to 6926 of SEQ ID NO: 1.

SEQ ID NO: 2 is identical to SEQ ID NO: 1 with the exception of the following mutations: nucleotide 1640 G>T, nucleotide 5279 G>A, nucleotide 6173 T>C. These mutations do not alter the encoded amino acid sequence, and thus the ABCA4 protein encoded by SEQ ID NO: 2 is identical to the ABCA4 protein encoded by SEQ ID NO: 1.

In some embodiments of the methods of the disclosure, the plasmid vector comprising an exogenous sequence further comprises a sequence encoding a 5′ inverted terminal repeat (ITR) and a sequence encoding a 3′ ITR. In some embodiments, the sequence encoding the 5′ ITR and the sequence encoding the 3′ ITR are derived from a 5′ITR sequence and a 3′ ITR sequence of an AAV of serotype 2 (AAV2). In some embodiments, the sequence encoding the 5′ ITR and the sequence encoding the 3′ ITR comprise sequences that are identical to a sequence of a 5′ITR and a sequence of a 3′ ITR of an AAV2. In some embodiments, the sequence encoding the 5′ ITR comprises or consists of the nucleotide sequence of:

(SEQ ID NO: 34)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT.

In some embodiments, the sequence encoding the 3′ ITR comprises or consists of the nucleotide sequence of:

(SEQ ID NO: 35)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

In some embodiments of the compositions of the disclosure, the polynucleotide further comprises a Kozak sequence. In some embodiments, the Kozak sequence comprises or consists of the nucleotide sequence of GGCCACCATG (SEQ ID NO: 73).

In some embodiments of the compositions of the disclosure, the polynucleotide comprises or consists of the sequence of:

| (SEQ ID NO: 74) | |
|---|---|
| 1 | CTGCGCGCTC GCTCGCTCAC TGAGGCCGCC CGGGCGTCGG GCGACCTTTG GTCGCCCGGC |
| 61 | CTCAGTGAGC GAGCGAGCGC GCAGAGAGGG AGTGGCCAAC TCCATCACTA GGGGTTCCTG |
| 121 | CGGCAATTCA GTCGATAACT ATAACGGTCC TAAGGTAGCG ATTTAAATAC GCGCTCTCTT |
| 181 | AAGGTAGCCC CGGGACGCGT CAATTGGGGC CCCAGAAGCC TGGTGGTTGT TTGTCCTTCT |
| 241 | CAGGGGAAAA GTGAGGCGGC CCCTTGGAGG AAGGGGCCGG GCAGAATGAT CTAATCGGAT |
| 301 | TCCAAGCAGC TCAGGGGATT GTCTTTTTCT AGCACCTTCT TGCCACTCCT AAGCGTCCTC |
| 361 | CGTGACCCCG GCTGGGATTT AGCCTGGTGC TGTGTCAGCC CCGGGGCCAC CATGAGAGAG |
| 421 | CCAGAGGAGC TGATGCCAGA CAGTGGAGCA GTGTTTACAT TCGGAAAATC TAAGTTCGCT |
| 481 | GAAAATAACC CAGGAAAGTT CTGGTTTAAA AACGACGTGC CCGTCCACCT GTCTTGTGGC |
| 541 | GATGAGCATA GTGCCGTGGT CACTGGGAAC AATAAGCTGT ACATGTTCGG GTCCAACAAC |
| 601 | TGGGGACAGC TGGGGCTGGG ATCCAAATCT GCTATCTCTA AGCCAACCTG CGTGAAGGCA |
| 661 | CTGAAACCCG AGAAGGTCAA ACTGGCCGCT TGTGGCAGAA ACCACACTCT GGTGAGCACC |
| 721 | GAGGGCGGGA ATGTCTATGC CACCGGAGGC AACAATGAGG GACAGCTGGG ACTGGGGGAC |
| 781 | ACTGAGGAAA GGAATACCTT TCACGTGATC TCCTTCTTTA CATCTGAGCA TAAGATCAAG |
| 841 | CAGCTGAGCG CTGGCTCCAA CACATCTGCA GCCCTGACTG AGGACGGGCG CCTGTTCATG |
| 901 | TGGGGAGATA ATTCAGAGGG CCAGATTGGG CTGAAAAACG TGAGCAATGT GTGCGTCCCT |
| 961 | CAGCAGGTGA CCATCGGAAA GCCAGTCAGT TGGATTTCAT GTGGCTACTA TCATAGCGCC |
| 1021 | TTCGTGACCA CAGATGGCGA GCTGTACGTC TTTGGGGAGC CCGAAAACGG AAAACTGGGC |
| 1081 | CTGCCTAACC AGCTGCTGGG CAATCACCGG ACACCCCAGC TGGTGTCCGA GATCCCTGAA |
| 1141 | AAAGTGATCC AGGTCGCCTG CGGGGGAGAG CATACAGTGG TCCTGACTGA GAATGCTGTG |
| 1201 | TATACCTTCG GACTGGGCCA GTTTGGCCAG CTGGGGCTGG GAACCTTCCT GTTTGAGACA |
| 1261 | TCCGAACCAA AAGTGATCGA GAACATTCGC GACCAGACTA TCAGCTACAT TTCCTGCGGA |
| 1321 | GAGAATCACA CCGCACTGAT CACAGACATT GGCCTGATGT ATACCTTTGG CGATGGACGA |
| 1381 | CACGGGAAGC TGGGACTGGG ACTGGAGAAC TTCACTAATC ATTTTATCCC CACCCTGTGT |
| 1441 | TCTAACTTCC TGCGGTTCAT CGTGAAACTG GTCGCTTGCG GCGGGTGTCA CATGGTGGTC |
| 1501 | TTCGCTGCAC CTCATAGGGG CGTGGCTAAG GAGATCGAAT TTGACGAGAT TAACGATACA |
| 1561 | TGCCTGAGCG TGGCAACTTT CCTGCCATAC AGCTCCCTGA CTTCTGGCAA TGTGCTGCAG |
| 1621 | AGAACCCTGA GTGCAAGGAT GCGGAGAAGG GAGAGGGAAC GCTCTCCTGA CAGTTTCTCA |
| 1681 | ATGCGACGAA CCCTGCCACC TATCGAGGGA ACACTGGGAC TGAGTGCCTG CTTCCTGCCT |
| 1741 | AACTCAGTGT TTCCACGATG TAGCGAGCGG AATCTGCAGG AGTCTGTCCT GAGTGAGCAG |
| 1801 | GATCTGATGC AGCCAGAGGA ACCCGACTAC CTGCTGGATG AGATGACCAA GGAGGCCGAA |
| 1861 | ATCGACAACT CTAGTACAGT GGAGTCCCTG GGCGAGACTA CCGATATCCT GAATATGACA |
| 1921 | CACATTATGT CACTGAACAG CAATGAGAAG AGTCTGAAAC TGTCACCAGT GCAGAAGCAG |
| 1981 | AAGAAACAGC AGACTATTGG CGAGCTGACT CAGGACACCG CCCTGACAGA GAACGACGAT |
| 2041 | AGCGATGAGT ATGAGGAAAT GTCCGAGATG AAGGAAGGCA AAGCTTGTAA GCAGCATGTC |
| 2101 | AGTCAGGGGA TCTTCATGAC ACAGCCAGCC ACAACTATTG AGGCTTTTTC AGACGAGGAA |
| 2161 | GTGGAGATCC CCGAGGAAAA AGAGGGCGCA GAAGATTCCA AGGGGAATGG AATTGAGGAA |
| 2221 | CAGGAGGTGG AAGCCAACGA GGAAAATGTG AAAGTCCACG GAGGCAGGAA GGAGAAAACA |
| 2281 | GAAATCCTGT CTGACGATCT GACTGACAAG GCCGAGGTGT CCGAAGGCAA GGCAAAATCT |
| 2341 | GTCGGAGAGG CAGAAGACGG ACCAGAGGGA CGAGGGGATG GAACCTGCGA GGAAGGCTCA |
| 2401 | AGCGGGGCTG AGCATTGGCA GGACGAGGAA CGAGAGAAGG GCGAAAAGGA TAAAGGCCGC |
| 2461 | GGGGAGATGG AACGACCTGG AGAGGGCGAA AAAGAGCTGG CAGAGAAGGA GGAATGGAAG |
| 2521 | AAAAGGGACG GCGAGGAACA GGAGCAGAAA GAAAGGGAGC AGGGCCACCA GAAGGAGCGC |
| 2581 | AACCAGGAGA TGGAAGAGGG CGGCGAGGAA GAGCATGGCG AGGGAGAAGA GGAAGAGGGC |
| 2641 | GATAGAGAAG AGGAAGAGGA AAAAGAAGGC GAAGGGAAGG AGGAAGGAGA GGGCGAGGAA |
| 2701 | GTGGAAGGCG AGAGGGAAAA GGAGGAAGGA GAACGGAAGA AAGAGGAAAG AGCCGGCAAA |
| 2761 | GAGGAAAAGG GCGAGGAAGA GGGCGATCAG GGCGAAGGCG AGGAGGAAGA GACCGAGGGC |
| 2821 | CGCGGGGAAG AGAAAGAGGA GGGAGGAGAG GTGGAGGGCG GAGAGGTCGA AGAGGGAAAG |
| 2881 | GGCGAGCGCG AAGAGGAAGA GGAAGAGGGC GAGGGCGAGG AAGAAGAGGG CGAGGGGGAA |
| 2941 | GAAGAGGAGG GAGAGGGCGA AGAGGAAGAG GGGGAGGGAA AGGGCGAAGA GGAAGGAGAG |
| 3001 | GAAGGGGAGG GAGAGGAAGA GGGGGAGGAG GGCGAGGGGG AAGGCGAGGA GGAAGAAGGA |
| 3061 | GAGGGGGAAG GCGAAGAGGA AGGCGAGGGG GAAGGAGAGG AGGAAGAAGG GGAAGGCGAA |
| 3121 | GGCGAAGAGG AGGGAGAAGG AGAGGGGGAG GAAGAGGAAG GAGAAGGGAA GGGCGAGGAG |
| 3181 | GAAGGCGAAG AGGGAGAGGG GGAAGGCGAG GAAGAGGAAG GCGAGGGCGA AGGAGAGGAC |
| 3241 | GGCGAGGGCG AGGGAGAAGA GGAGGAAGGG GAATGGGAAG GCGAAGAAGA GGAAGGCGAA |
| 3301 | GGCGAAGGCG AAGAAGAGGG CGAAGGGGAG GGCGAGGAGG GCGAAGGCGA AGGGGAGGAA |
| 3361 | GAGGAAGGCG AAGGAGAAGG CGAGGAAGAA GAGGGAGAGG AGGAAGGCGA GGAGGAAGGA |
| 3421 | GAGGGGGAGG AGGAGGGAGA AGGCGAGGGC GAAGAAGAAG AAGAGGGAGA AGTGGAGGGC |
| 3481 | GAAGTCGAGG GGGAGGAGGG AGAAGGGGAA GGGGAGGAAG AAGAGGGCGA AGAAGAAGGC |
| 3541 | GAGGAAAGAG AAAAAGAGGG AGAAGGCGAG GAAAACCGGA GAAATAGGGA AGAGGAGGAA |
| 3601 | GAGGAAGAGG GAAAGTACCA GGAGACAGGC GAAGAGGAAA ACGAGCGGCA GGATGGCGAG |
| 3661 | GAATATAAGA AAGTGAGCAA GATCAAAGGA TCCGTCAAGT ACGGCAAGCA CAAAACCTAT |
| 3721 | CAGAAGAAAA GCGTGACCAA CACACAGGGG AATGGAAAAG AGCAGAGGAG TAAGATGCCT |
| 3781 | GTGCAGTCAA AACGGCTGCT GAAGAATGGC CCATCTGGAA GTAAAAAATT CTGGAACAAT |
| 3841 | GTGCTGCCCC ACTATCTGGA ACTGAAATAA GAGCTCCTCG AGGCGGCCCG CTCGAGTCTA |
| 3901 | GAGGGCCCTT CGAAGGTAAG CCTATCCCTA ACCCTCTCCT CGGTCTCGAT TCTACGCGTA |
| 3961 | CCGGTCATCA TCACCATCAC CATTGAGTTT AAACCCGCTG ATCAGCCTCG ACTGTGCCTT |
| 4021 | CTAGTTGCCA GCCATCTGTT GTTTGCCCCT CCCCCGTGCC TTCCTTGACC CTGGAAGGTG |
| 4081 | CCACTCCCAC TGTCCTTTCC TAATAAAATG AGGAAATTGC ATCGCATTGT CTGAGTAGGT |
| 4141 | GTCATTCTAT TCTGGGGGGT GGGGTGGGGC AGGACAGCAA GGGGGAGGAT TGGGAAGACA |
| 4201 | ATAGCAGGCA TGCTGGGGAT GCGGTGGGCT CTATGGCTTC TGAGGCGGAA AGAACCAGAT |
| 4261 | CCTCTCTTAA GGTAGCATCG AGATTTAAAT TAGGGATAAC AGGGTAATGG CGCGGGCCGC |
| 4321 | AGGAACCCCT AGTGATGGAG TTGGCCACTC CCTCTCTGCG CGCTCGCTCG CTCACTGAGG |
| 4381 | CCGGGCGACC AAAGGTCGCC CGACGCCCGG GCTTTGCCCG GGCGGCCTCA GTGAGCGAGC |
| 4441 | GAGCGCGCAG. |

In some embodiments of the methods of the disclosure, the plasmid vector comprising an exogenous sequence, the helper plasmid vector or the plasmid vector comprising the sequence encoding a viral Rep protein and a viral Cap protein further comprises a sequence encoding a selection marker.

In some embodiments of the methods of the disclosure, the sequence encoding the viral Rep protein and the sequence encoding the viral Cap protein comprise sequences isolated or derived from AAV serotype 8 (AAV8) viral Rep protein and viral Cap protein sequences.

In some embodiments of the methods of the disclosure, the harvest media comprises DMEM, 4 mM stabilized glutamine or stabilized glutamine dipeptide, and Benzonase.

In some embodiments of the methods of the disclosure, the mammalian host cells have been transfected with a composition comprising one or more of a polymer (e.g. a polyethylenimine (PEI) composition), calcium phosphate, a lipid, a vector capable of traversing a cell membrane (e.g. a liposome, a micelle, a nanoparticle (e.g. carbon, silicon, polymer and gold). In some embodiments, the mammalian host cells have been transfected with a composition comprising polyethylenimine (PEI) (i.e. a PEI composition).

In some embodiments of the methods of the disclosure, the virus release solution comprises a salt and a high pH. In some embodiments, the salt comprises NaCl. In some embodiments, the high pH is a basic pH. In some embodiments, the high pH is greater than 7.0. In some embodiments, high pH comprises a pH greater than or equal to 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0.

In some embodiments of the methods of the disclosure, the conditions suitable for the formation of a plurality of rAAV particles comprise incubating the mammalian host cells at conditions recapitulating in vivo physiology for at least 1, 2, 3, 4, 5, 6, 7, 8, 9, 10, 11, 12, 13, 14, 15, 16, 17, 18, 19, 20, 21, 22, 23, or 24 hours. In some embodiments, conditions recapitulating in vivo physiology include 5% CO2 at a temperature that is minimally human internal body temperature. In some embodiments, conditions suitable for the formation of a plurality of rAAV particles comprises incubating the mammalian host cells at a CO2 level equal to or less than 10% CO2. In some embodiments, human internal body temperature is at least 36° C.

In some embodiments of the methods of the disclosure, the HIC step of (a) further comprises the steps of: (i) generating a HIC chromatogram; and (ii) selecting a fraction on the HIC chromatogram containing rAAV particles to produce the HIC eluate comprising a plurality of rAAV viral particles. In some embodiments, the HIC step further comprises diluting the harvest media into a high salt buffer prior to generating the HIC chromatogram. In some embodiments, the plurality of rAAV particles are eluted using a step gradient. In some embodiments, the step gradient comprises a decrease in salt concentration at each step gradient. In some embodiments of the methods of the disclosure, the CEX step of (b) further comprises the steps of: (i) generating a CEX chromatogram; and (ii) selecting a fraction from the CEX chromatogram containing rAAV particles to produce the CEX eluate comprising a plurality of rAAV viral particles. In some embodiments, the CEX chromatography comprises an SO₃− cation exchange matrix. In some embodiments, the CEX chromatography step further comprises adjusting the HIC eluate into a low salt buffer prior to generating the CEX chromatogram. In some embodiments, the adjustment comprises a dilution step. In some embodiments, the adjustment step comprises a TFF step. In some embodiments, the TFF step is performed using a 100 kDa hollow fiber filter (HFF). In some embodiments, the TFF step is performed using at least a 70 kDa HFF. In some embodiments, the TFF step is performed using at least a 50 kDa HFF. In some embodiments, the TFF step is performed using at least a 50, 55, 60, 65, 70, 75, 80, 85, 90, 95, or 100 kDa HFF or any number of kDa in between. In some embodiments, the pH of the HIC eluate is adjusted to pH 3.0 to pH 4.0, inclusive of the endpoints. In some embodiments, the pH of the HIC eluate is adjusted to pH 3.5 to pH 3.7, inclusive of the endpoints. In some embodiments, the CEX step further comprises filtering the HIC eluate. In some embodiments, filtering the HIC eluate comprises a 0.8/0.45 μm polyethersulfone (PES) filter. In some embodiments, the plurality of rAAV particles are eluted using a step gradient. In some embodiments, the step gradient comprises a pH gradient, a salt gradient or a combination thereof. In some embodiments, the plurality of rAAV particles are eluted using a linear gradient. In some embodiments, the linear gradient comprises a pH gradient, a salt gradient or a combination thereof. In some embodiments, the CEX step further comprises neutralizing the pH of the CEX eluate. In some embodiments, the pH of the neutralized CEX eluate is pH 9.0.

In some embodiments of the methods of the disclosure, the AEX Chromatography step of (c) further comprises the steps of: (i) generating an AEX chromatogram; and (ii) selecting a fraction from the AEX chromatogram containing full rAAV particles to produce the AEX eluate comprising a purified and enriched plurality of full rAAV particles. In some embodiments, the AEX chromatography comprises an Anion Exchange (QA) matrix. In some embodiments, the AEX chromatography step further comprises diluting the CEX eluate into a low salt buffer prior to generating the AEX chromatogram. In some embodiments, the adjustment comprises a dilution step. In some embodiments, the adjustment step comprises a TFF step. In some embodiments, the adjustment step comprises a first TFF step and a second TFF step. In some embodiments, the TFF step is performed using a 100 kDa hollow fiber filter (HFF). In some embodiments, the diluted CEX eluate is pH 9.0. In some embodiments, the purified and enriched plurality of full rAAV particles are eluted using a linear gradient. In some embodiments, the purified and enriched plurality of full rAAV particles are eluted using a step gradient. In some embodiments, the CEX step further comprises neutralizing the pH of the eluate comprising the purified and enriched plurality of full rAAV particles.

In some embodiments of the methods of the disclosure, the TFF step of (d) is performed using a 100 kDa hollow fiber filter (HFF). In some embodiments, step (f) the method further comprises a second TFF step, and wherein both the first and second TFF steps are performed using a 100 kDa HFF. In some embodiments, the final formulation buffer comprises Tris, MgCl₂, and NaCl. In some embodiments, the final formulation buffer comprises 20 mM Tris, 1 mM MgCl₂, and 200 mM NaCl at pH 8. In some embodiments, the final formulation buffer further comprises poloxamer 188 at 0.001%.

In some embodiments of the methods of the disclosure, the methods further comprise adding poloxamer 188 to the final composition.

In some embodiments of the methods of the disclosure, the final composition comprising the purified and enriched plurality of full rAAV particles and the final formulation buffer is frozen at −80° C.

The disclosure provides a composition comprising a plurality of rAAV particles produced by a method of the disclosure.

In some embodiments of the compositions of the disclosure, the composition comprises (a) between 0.5×10¹¹ vg/mL and 1×10¹³ vg/mL, inclusive of the endpoints and (b) less than 50% empty capsids. In some embodiments of the compositions of the disclosure, the composition comprises (a) between 0.5×10¹¹ vg/mL and 1×10¹³ vg/mL, or between 1×10¹¹ vg/mL and 1×10¹³ vg/mL, inclusive of the endpoints and (b) less than 30% empty capsids. In some embodiments, the composition comprises (a) between 0.5×10¹¹ vg/mL and 1×10¹³ vg/mL, or between 1×10¹¹ vg/mL and 1×10¹³ vg/mL, inclusive of the endpoints and (b) less than 25% empty capsids In some embodiments, the composition comprises (a) between 0.5×10¹¹ vg/mL and 1×10¹³ vg/mL, or between 1×10¹¹ vg/mL and 1×10¹³ vg/mL, inclusive of the endpoints and (b) less than 99%, 97%, 95%, 90%, 85%, 80%, 75%, 70%, 65%, 60%, 55%, 50%, 45%, 40%, 35%, 30%, 25%, 20%, 15%, 10%, 5%, 2%, 1%, or any percentage in between of empty capsids. In some embodiments, the composition comprises about 5×10¹² vg/mL.

In some embodiments of the compositions of the disclosure, the composition comprises (a) between 0.5×10¹¹ vg/mL and 1×10¹³ vg/mL, or between 1×10¹¹ vg/mL and 1×10¹³ vg/mL, inclusive of the endpoints and (b) at least 70% full capsids. In some embodiments, the composition comprises (a) between 0.5×10¹¹ vg/mL and 1×10¹³ vg/mL, or between 1×10¹¹ vg/mL and 1×10¹³ vg/mL, inclusive of the endpoints and (b) at least 1%, 2%, 5%, 10%, 15%, 20%, 25%, 30%, 35%, 40%, 45%, 50%, 55%, 60%, 65%, 70%, 75%, 80%, 85%, 90%, 95%, 97%, 99%, 100%, or any percentage in between of full capsids. In some embodiments, the composition comprises 5×10¹² vg/mL.

In some embodiments of the compositions of the disclosure, a portion of the plurality of rAAV comprises a functional vector genome, wherein each functional vector genome is capable of expressing an exogenous sequence in a cell following transduction. In some embodiments, the portion of the plurality of rAAV comprising a functional vector genome expresses the exogenous sequence at a 2-fold increase when compared to a level of expression of a corresponding endogenous sequence in a nontransduced cell. In some embodiments, the portion of the plurality of rAAV comprising a functional vector genome expresses the exogenous sequence at a 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or any other increment fold increase in between, when compared to a level of expression of a corresponding endogenous sequence in a nontransduced cell.

In some embodiments of the compositions of the disclosure, including those wherein a portion of the plurality of rAAV comprises a functional vector genome, wherein each functional vector genome is capable of expressing an exogenous sequence in a cell following transduction, the exogenous sequence and the corresponding endogenous sequence are not identical. In some embodiments, the exogenous sequence and the corresponding endogenous sequence are not identical, but a protein encoded by the exogenous sequence and a protein encoded by the endogenous sequence are identical. In some embodiments, the exogenous sequence and the corresponding endogenous sequence have at least 70%, 75%, 80%, 85%, 90%, 95%, 97%, 99% or any percentage in between of identity. In some embodiments, the exogenous sequence is codon-optimized when compared to the endogenous sequence. In some embodiments, the exogenous sequence and the corresponding endogenous sequence have at least 50%, 55%, 60%, 65%, 70%, 75%, 80%, 85%, 90%, 95%, 97%, 99% or any percentage in between of identity. In some embodiments of the composition of the disclosure, following transduction of a cell with a composition of the disclosure, the exogenous sequence encodes a protein. In some embodiments, the protein encoded by the exogenous sequence has an activity level equal to or greater than an activity level of a protein encoded by a corresponding sequence of a nontransduced cell. In some embodiments, the exogenous sequence and the corresponding endogenous sequence are identical. In some embodiments, the exogenous sequence and the corresponding endogenous sequence are not identical. In some embodiments, the exogenous sequence and the corresponding endogenous sequence have at least 70%, 75%, 80%, 85%, 90%, 95%, 97%, 99% or any percentage in between of identity. In some embodiments, following transduction of a cell with a composition of the disclosure, the exogenous sequence encodes a protein.

In some embodiments of the methods of the disclosure, including those wherein the method comprises the step of culturing a plurality of mammalian host cells in a harvest media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells, prior to the contacting step, the plasmid vector comprising an exogenous sequence, the helper plasmid vector, and the plasmid vector comprising a sequence encoding a viral Rep protein and a viral Cap protein are provided at a molar ratio of about 0.5:1:1 to about 10:1:1, about 1:1:1 to about 10:1:1, about 2:1:1 to about 10:1:1, or about 3:1:1 to about 10:1:1, respectively, optionally about 0.5:1:1, about 1:1:1, about 2:1:1, about 3:1:1, about 4:1:1, about 5:1:1, about 6:1:1, about 7:1:1, about 8:1:1, about 9:1:1, or about 10:1:1. In some embodiments, the plasmid vector comprising an exogenous sequence, the helper plasmid vector, and the plasmid vector comprising a sequence encoding a viral Rep protein and a viral Cap protein are provided in a molar ratio of about 1:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 3:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 10:1:1, respectively.

In some embodiments of the methods of the disclosure, including those wherein the method comprises the step of culturing a plurality of mammalian host cells in a harvest media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells, prior to the contacting step, the plasmid vector comprising an exogenous sequence (pITR) and the helper plasmid vector (pHELP) is provided in a molar ratio of between 1:1 and 20:19 or between 1:20 and 20:1, or between 1:20 and 1:1 (e.g., any of the ratios shown below in Table A). In some embodiments, the molar ratio of pITR and the plasmid vector comprising a sequence encoding a viral Rep protein and a viral Cap protein (pREPCAP) is between 1:1 and 20:19, or between 1:20 and 20:1, or between 1:20 and 1:1 (e.g., any of the ratios shown below in Table A). In some embodiments, the plasmid vector comprising an exogenous sequence, the helper plasmid vector, and the plasmid vector comprising a sequence encoding a viral Rep protein and a viral Cap protein are provided in a molar ratio of about 3:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 10:1:1, respectively. In certain embodiments, the transfection is conducted using CaPO₄ or PEI. In particular embodiments, the transfection is conducted using PEI at a PEI:DNA ratio (mL:mg) of about 1:1 to about 5:1, respectively, optionally about 2:1 to about 4:1, about 4:1, about 3:1, or about 2:1. In certain embodiments, the transection is conducted using PEI, wherein the plasmid vector comprising an exogenous sequence, the helper plasmid vector, and the plasmid vector comprising a sequence encoding a viral Rep protein and a viral Cap protein are provided in a molar ratio of about 1:1:1, respectively. In certain embodiments, the transfection is conducted using PEI at a PEI:DNA ratio (mL:mg) of about 0.5:1 to 5:1 or about 1:1 to about 5:1, respectively, optionally about 2:1 to about 4:1, about 4:1, about 3:1, or about 2:1, wherein the plasmid vector comprising an exogenous sequence, the helper plasmid vector, and the plasmid vector comprising a sequence encoding a viral Rep protein and a viral Cap protein are provided in a molar ratio of about 0.5:1:1 to about 10:1:1, about 1:1:1 to about 10:1:1, about 2:1:1 to about 10:1:1 optionally about 0.5:1:1, about 1:1:1, about 2:1:1, about 3:1:1, about 4:1:1, about 5:1:1, about 6:1:1, about 7:1:1, about 8:1:1, about 9:1:1, or about 10:1:1. In some embodiments, the plasmid vector comprising an exogenous sequence, the helper plasmid vector, and the plasmid vector comprising a sequence encoding a viral Rep protein and a viral Cap protein are provided in a molar ratio of about 1:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 3:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 10:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 2:1:1, about 3:1:1, about 4:1:1, about 5:1:1, about 6:1:1, about 7:1:1, about 8:1:1, or about 9:1:1, respectively.

TABLE A
Molar ratio of pHELP and/or pREPCAP v pITR
pHELP and/or pREPCAP

| | | | | | | | | | | | | | | | | | | | |
|---|---|---|---|---|---|---|---|---|---|---|---|---|---|---|---|---|---|---|---|
| 1 | | | | | | | | | | | | | | | | | | | |
| 2 | 2:1 | | | | | | | | | | | | | | | | | | |
| 3 | 3:1 | 3:2 | | | | | | | | | | | | | | | | | |
| 4 | 4:1 | 4:2 | 4:3 | | | | | | | | | | | | | | | | |
| 5 | 5:1 | 5:2 | 5:3 | 5:4 | | | | | | | | | | | | | | | |
| 6 | 6:1 | 6:2 | 6:3 | 6:4 | 6:5 | | | | | | | | | | | | | | |
| 7 | 7:1 | 7:2 | 7:3 | 7:4 | 7:5 | 7:6 | | | | | | | | | | | | | |
| 8 | 8:1 | 8:2 | 8:3 | 8:4 | 8:5 | 8:6 | 8:7 | | | | | | | | | | | | |
| 9 | 9:1 | 9:2 | 9:3 | 9:4 | 9:5 | 9:6 | 9:7 | 9:8 | | | | | | | | | | | |
| 10 | 10:1 | 10:2 | 10:3 | 10:4 | 10:5 | 10:6 | 10:7 | 10:8 | 10:9 | | | | | | | | | | |
| 11 | 11:1 | 11:2 | 11:3 | 11:4 | 11:5 | 11:6 | 11:7 | 11:8 | 11:9 | 11:10 | | | | | | | | | |
| 12 | 12:1 | 12:2 | 12:3 | 12:4 | 12:5 | 12:6 | 12:7 | 12:8 | 12:9 | 12:10 | 12:11 | | | | | | | | |
| 13 | 13:1 | 13:2 | 13:3 | 13:4 | 13:5 | 13:6 | 13:7 | 13:8 | 13:9 | 13:10 | 13:11 | 13:12 | | | | | | | |
| 14 | 14:1 | 14:2 | 14:3 | 14:4 | 14:5 | 14:6 | 14:7 | 14:8 | 14:9 | 14:10 | 14:11 | 14:12 | 14:13 | | | | | | |
| 15 | 15:1 | 15:2 | 15:3 | 15:4 | 15:5 | 15:6 | 15:7 | 15:8 | 15:9 | 15:10 | 15:11 | 15:12 | 15:13 | 15:14 | | | | | |
| 16 | 16:1 | 16:2 | 16:3 | 16:4 | 16:5 | 16:6 | 16:7 | 16:8 | 16:9 | 16:10 | 16:11 | 16:12 | 16:13 | 16:14 | 16:15 | | | | |
| 17 | 17:1 | 17:2 | 17:3 | 17:4 | 17:5 | 17:6 | 17:7 | 17:8 | 17:9 | 17:10 | 17:11 | 17:12 | 17:13 | 17:14 | 17:15 | 17:16 | | | |
| 18 | 18:1 | 18:2 | 18:3 | 18:4 | 18:5 | 18:6 | 18:7 | 18:8 | 18:9 | 18:10 | 18:11 | 18:12 | 18:13 | 18:14 | 18:15 | 18:16 | 18:17 | | |
| 19 | 19:1 | 19:2 | 19:3 | 19:4 | 19:5 | 19:6 | 19:7 | 19:8 | 19:9 | 19:10 | 19:11 | 19:12 | 19:13 | 19:14 | 19:15 | 19:16 | 19:17 | 19:18 | |
| 20 | 20:1 | 20:2 | 20:3 | 20:4 | 20:5 | 20:6 | 20:7 | 20:8 | 20:9 | 20:10 | 20:11 | 20:12 | 20:13 | 20:14 | 20:15 | 20:16 | 20:17 | 20:18 | 20:19 |

In some embodiments of the methods of the disclosure, including those wherein the method comprises the step of culturing a plurality of mammalian host cells in a harvest media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells, prior to the contacting step, and in which a molar ratio of the plasmid vector to either the helper plasmid vector or the RepCap vector comprises a greater value for the plasmid vector than either the helper plasmid vector or the RepCap vector, the culturing a plurality of mammalian host cells in a harvest media under conditions suitable for the formation of a plurality of rAAV particles comprises a transfection agent. In some embodiments, the transfection agent comprises polyethylenimine. In some embodiments, the transfection agent comprises calcium phosphate (CaPO₄).

In certain related embodiments, the disclosure provides a method of producing a recombinant AAV vector, comprising transfecting mammalian host cells with: (i) a plasmid vector comprising an exogenous sequence; (ii) a plasmid vector comprising a sequence encoding a viral Rep protein and a viral Cap protein; and (iii) a helper plasmid vector, wherein the mammalian host cells are contacted with a transfection medium comprising the plasmid vector comprising the exogenous sequence, the plasmid vector comprising a sequence encoding a viral Rep protein and a viral Cap protein, and the helper plasmid at a molar ratio of about 0.5:1:1 to about 10:1:1, or about 1:1:1 to about 10:1:1, respectively, optionally about 2:1:1, about 3:1:1, about 4:1:1, about 5:1:1, about 6:1:1, about 7:1:1, about 8:1:1, about 9:1:1, or about 10:1:1. In some embodiments, the transfection medium comprises a transfection agent selected from polyethylenimine (PEI) and CaPO₄. In certain embodiments, the transfection agent is PEI, and wherein the tranfection medium comprises PEI and DNA at a ratio of about 5:1 to about 1:1, about 2:1 to about 4:1, about 4:1, about 3:1, about 2:1, or about 1:1.

In particular embodiments of the methods of producing a recombinant AAV vector disclosed herein, the exogenous sequence comprises: (a) a sequence encoding a rhodopsin kinase promoter; (b) a sequence encoding a retinitis pigmentosa GTPase regulator ORF15 isoform (RPGR^{ORF15}); and (c) a sequence encoding a polyadenylation (polyA) signal. In some embodiments, the rhodopsin kinase promoter is a GRK1 promoter, e.g., a GRK1 promoter comprising or consisting of:

| (SEQ ID NO: 5) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments, the sequence encoding the RPGRORF15 is a codon optimized human RPGRORF15 sequence, including but not limited to any of those disclosed herein.

In particular embodiments of the methods of producing a recombinant AAV vector disclosed herein, the sequence encoding the polyA signal comprises a bovine growth hormone (BGH) polyA sequence, including but not limited to any of those disclosed herein.

In particular embodiments of the methods of producing a recombinant AAV vector disclosed herein, the plasmid vector comprising an exogenous sequence further comprises a sequence encoding a 5′ inverted terminal repeat (ITR) and a sequence encoding a 3′ ITR. In some embodiments, the sequence encoding the 5′ ITR and the sequence encoding the 3′ ITR are derived from a 5′ITR sequence and a 3′ ITR sequence of an AAV of serotype 2 (AAV2). In some embodiments, the sequence encoding the 5′ ITR and the sequence encoding the 3′ ITR comprise sequences that are identical to a sequence of a 5′ITR and a sequence of a 3′ ITR of an AAV2. In other embodiments, the ITRs comprise one or more modifications as compared to a wild type AAV2, e.g., one or more nucleotide deletions, insertions or substitutions. In certain embodiments, the ITRs are derived from a 3′ AAV2 ITR in forward and reverse orientation with subsequent deletions to produce stabilized ITRs. In certain embodiment, the sequence encoding the 5′ ITR comprises or consists of the nucleotide sequence of:

(SEQ ID NO: 34)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT.

In certain embodiments, the sequence encoding the 3′ ITR comprises or consists of the nucleotide sequence of:

(SEQ ID NO: 35)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

In particular embodiments of the methods of producing a recombinant AAV vector disclosed herein, the exogenous sequence further comprises a sequence encoding a Kozak sequence. In certain embodiments, the Kozak sequence comprises the nucleotide sequence of GGCCACCATG (SEQ ID NO: 73).

In particular embodiments of the methods of producing a recombinant AAV vector disclosed herein, the exogenous sequence comprises the sequence of:

| (SEQ ID NO: 74) | |
|---|---|
| 1 | CTGCGCGCTC GCTCGCTCAC TGAGGCCGCC CGGGCGTCGG GCGACCTTTG GTCGCCCGGC |
| 61 | CTCAGTGAGC GAGCGAGCGC GCAGAGAGGG AGTGGCCAAC TCCATCACTA GGGGTTCCTG |
| 121 | CGGCAATTCA GTCGATAACT ATAACGGTCC TAAGGTAGCG ATTTAAATAC GCGCTCTCTT |
| 181 | AAGGTAGCCC CGGGACGCGT CAATTGGGGC CCCAGAAGCC TGGTGGTTGT TTGTCCTTCT |
| 241 | CAGGGGAAAA GTGAGGCGGC CCCTTGGAGG AAGGGGCCGG GCAGAATGAT CTAATCGGAT |
| 301 | TCCAAGCAGC TCAGGGGATT GTCTTTTTCT AGCACCTTCT TGCCACTCCT AAGCGTCCTC |
| 361 | CGTGACCCCG GCTGGGATTT AGCCTGGTGC TGTGTCAGCC CCGGGGCCAC CATGAGAGAG |
| 421 | CCAGAGGAGC TGATGCCAGA CAGTGGAGCA GTGTTTACAT TCGGAAAATC TAAGTTCGCT |
| 481 | GAAAATAACC CAGGAAAGTT CTGGTTTAAA AACGACGTGC CCGTCCACCT GTCTTGTGGC |
| 541 | GATGAGCATA GTGCCGTGGT CACTGGGAAC AATAAGCTGT ACATGTTCGG GTCCAACAAC |
| 601 | TGGGGACAGC TGGGGCTGGG ATCCAAATCT GCTATCTCTA AGCCAACCTG CGTGAAGGCA |
| 661 | CTGAAACCCG AGAAGGTCAA ACTGGCCGCT TGTGGCAGAA ACCACACTCT GGTGAGCACC |
| 721 | GAGGGCGGGA ATGTCTATGC CACCGGAGGC AACAATGAGG GACAGCTGGG ACTGGGGGAC |
| 781 | ACTGAGGAAA GGAATACCTT TCACGTGATC TCCTTCTTTA CATCTGAGCA TAAGATCAAG |
| 841 | CAGCTGAGCG CTGGCTCCAA CACATCTGCA GCCCTGACTG AGGACGGGCG CCTGTTCATG |
| 901 | TGGGGAGATA ATTCAGAGGG CCAGATTGGG CTGAAAAACG TGAGCAATGT GTGCGTCCCT |
| 961 | CAGCAGGTGA CCATCGGAAA GCCAGTCAGT TGGATTTCAT GTGGCTACTA TCATAGCGCC |
| 1021 | TTCGTGACCA CAGATGGCGA GCTGTACGTC TTTGGGGAGC CCGAAAACGG AAAACTGGGC |
| 1081 | CTGCCTAACC AGCTGCTGGG CAATCACCGG ACACCCCAGC TGGTGTCCGA GATCCCTGAA |
| 1141 | AAAGTGATCC AGGTCGCCTG CGGGGGAGAG CATACAGTGG TCCTGACTGA GAATGCTGTG |
| 1201 | TATACCTTCG GACTGGGCCA GTTTGGCCAG CTGGGGCTGG GAACCTTCCT GTTTGAGACA |
| 1261 | TCCGAACCAA AAGTGATCGA GAACATTCGC GACCAGACTA TCAGCTACAT TTCCTGCGGA |
| 1321 | GAGAATCACA CCGCACTGAT CACAGACATT GGCCTGATGT ATACCTTTGG CGATGGACGA |
| 1381 | CACGGGAAGC TGGGACTGGG ACTGGAGAAC TTCACTAATC ATTTTATCCC CACCCTGTGT |
| 1441 | TCTAACTTCC TGCGGTTCAT CGTGAAACTG GTCGCTTGCG GCGGGTGTCA CATGGTGGTC |
| 1501 | TTCGCTGCAC CTCATAGGGG CGTGGCTAAG GAGATCGAAT TTGACGAGAT TAACGATACA |
| 1561 | TGCCTGAGCG TGGCAACTTT CCTGCCATAC AGCTCCCTGA CTTCTGGCAA TGTGCTGCAG |
| 1621 | AGAACCCTGA GTGCAAGGAT GCGGAGAAGG GAGAGGGAAC GCTCTCCTGA CAGTTTCTCA |
| 1681 | ATGCGACGAA CCCTGCCACC TATCGAGGGA ACACTGGGAC TGAGTGCCTG CTTCCTGCCT |
| 1741 | AACTCAGTGT TTCCACGATG TAGCGAGCGG AATCTGCAGG AGTCTGTCCT GAGTGAGCAG |
| 1801 | GATCTGATGC AGCCAGAGGA ACCCGACTAC CTGCTGGATG AGATGACCAA GGAGGCCGAA |
| 1861 | ATCGACAACT CTAGTACAGT GGAGTCCCTG GGCGAGACTA CCGATATCCT GAATATGACA |
| 1921 | CACATTATGT CACTGAACAG CAATGAGAAG AGTCTGAAAC TGTCACCAGT GCAGAAGCAG |
| 1981 | AAGAAACAGC AGACTATTGG CGAGCTGACT CAGGACACCG CCCTGACAGA GAACGACGAT |
| 2041 | AGCGATGAGT ATGAGGAAAT GTCCGAGATG AAGGAAGGCA AAGCTTGTAA GCAGCATGTC |
| 2101 | AGTCAGGGGA TCTTCATGAC ACAGCCAGCC ACAACTATTG AGGCTTTTTC AGACGAGGAA |
| 2161 | GTGGAGATCC CCGAGGAAAA AGAGGGCGCA GAAGATTCCA AGGGGAATGG AATTGAGGAA |
| 2221 | CAGGAGGTGG AAGCCAACGA GGAAAATGTG AAAGTCCACG GAGGCAGGAA GGAGAAAACA |
| 2281 | GAAATCCTGT CTGACGATCT GACTGACAAG GCCGAGGTGT CCGAAGGCAA GGCAAAATCT |
| 2341 | GTCGGAGAGG CAGAAGACGG ACCAGAGGGA CGAGGGGATG GAACCTGCGA GGAAGGCTCA |
| 2401 | AGCGGGGCTG AGCATTGGCA GGACGAGGAA CGAGAGAAGG GCGAAAAGGA TAAAGGCCGC |
| 2461 | GGGGAGATGG AACGACCTGG AGAGGGCGAA AAAGAGCTGG CAGAGAAGGA GGAATGGAAG |
| 2521 | AAAAGGGACG GCGAGGAACA GGAGCAGAAA GAAAGGGAGC AGGGCCACCA GAAGGAGCGC |
| 2581 | AACCAGGAGA TGGAAGAGGG CGGCGAGGAA GAGCATGGCG AGGGAGAAGA GGAAGAGGGC |
| 2641 | GATAGAGAAG AGGAAGAGGA AAAAGAAGGC GAAGGGAAGG AGGAAGGAGA GGGCGAGGAA |
| 2701 | GTGGAAGGCG AGAGGGAAAA GGAGGAAGGA GAACGGAAGA AAGAGGAAAG AGCCGGCAAA |
| 2761 | GAGGAAAAGG GCGAGGAAGA GGGCGATCAG GGCGAAGGCG AGGAGGAAGA GACCGAGGGC |
| 2821 | CGCGGGGAAG AGAAAGAGGA GGGAGGAGAG GTGGAGGGCG GAGAGGTCGA AGAGGGAAAG |
| 2881 | GGCGAGCGCG AAGAGGAAGA GGAAGAGGGC GAGGGCGAGG AAGAAGAGGG CGAGGGGGAA |
| 2941 | GAAGAGGAGG GAGAGGGCGA AGAGGAAGAG GGGGAGGGAA AGGGCGAAGA GGAAGGAGAG |
| 3001 | GAAGGGGAGG GAGAGGAAGA GGGGGAGGAG GGCGAGGGGG AAGGCGAGGA GGAAGAAGGA |
| 3061 | GAGGGGGAAG GCGAAGAGGA AGGCGAGGGG GAAGGAGAGG AGGAAGAAGG GGAAGGCGAA |
| 3121 | GGCGAAGAGG AGGGAGAAGG AGAGGGGGAG GAAGAGGAAG GAGAAGGGAA GGGCGAGGAG |
| 3181 | GAAGGCGAAG AGGGAGAGGG GGAAGGCGAG GAAGAGGAAG GCGAGGGCGA AGGAGAGGAC |
| 3241 | GGCGAGGGCG AGGGAGAAGA GGAGGAAGGG GAATGGGAAG GCGAAGAAGA GGAAGGCGAA |
| 3301 | GGCGAAGGCG AAGAAGAGGG CGAAGGGGAG GGCGAGGAGG GCGAAGGCGA AGGGGAGGAA |
| 3361 | GAGGAAGGCG AAGGAGAAGG CGAGGAAGAA GAGGGAGAGG AGGAAGGCGA GGAGGAAGGA |
| 3421 | GAGGGGGAGG AGGAGGGAGA AGGCGAGGGC GAAGAAGAAG AAGAGGGAGA AGTGGAGGGC |
| 3481 | GAAGTCGAGG GGGAGGAGGG AGAAGGGGAA GGGGAGGAAG AAGAGGGCGA AGAAGAAGGC |
| 3541 | GAGGAAAGAG AAAAAGAGGG AGAAGGCGAG GAAAACCGGA GAAATAGGGA AGAGGAGGAA |
| 3601 | GAGGAAGAGG GAAAGTACCA GGAGACAGGC GAAGAGGAAA ACGAGCGGCA GGATGGCGAG |
| 3661 | GAATATAAGA AAGTGAGCAA GATCAAAGGA TCCGTCAAGT ACGGCAAGCA CAAAACCTAT |
| 3721 | CAGAAGAAAA GCGTGACCAA CACACAGGGG AATGGAAAAG AGCAGAGGAG TAAGATGCCT |
| 3781 | GTGCAGTCAA AACGGCTGCT GAAGAATGGC CCATCTGGAA GTAAAAAATT CTGGAACAAT |
| 3841 | GTGCTGCCCC ACTATCTGGA ACTGAAATAA GAGCTCCTCG AGGCGGCCCG CTCGAGTCTA |
| 3901 | GAGGGCCCTT CGAAGGTAAG CCTATCCCTA ACCCTCTCCT CGGTCTCGAT TCTACGCGTA |
| 3961 | CCGGTCATCA TCACCATCAC CATTGAGTTT AAACCCGCTG ATCAGCCTCG ACTGTGCCTT |
| 4021 | CTAGTTGCCA GCCATCTGTT GTTTGCCCCT CCCCCGTGCC TTCCTTGACC CTGGAAGGTG |
| 4081 | CCACTCCCAC TGTCCTTTCC TAATAAAATG AGGAAATTGC ATCGCATTGT CTGAGTAGGT |
| 4141 | GTCATTCTAT TCTGGGGGGT GGGGTGGGGC AGGACAGCAA GGGGGAGGAT TGGGAAGACA |
| 4201 | ATAGCAGGCA TGCTGGGGAT GCGGTGGGCT CTATGGCTTC TGAGGCGGAA AGAACCAGAT |
| 4261 | CCTCTCTTAA GGTAGCATCG AGATTTAAAT TAGGGATAAC AGGGTAATGG CGCGGGCCGC |
| 4321 | AGGAACCCCT AGTGATGGAG TTGGCCACTC CCTCTCTGCG CGCTCGCTCG CTCACTGAGG |
| 4381 | CCGGGCGACC AAAGGTCGCC CGACGCCCGG GCTTTGCCCG GGCGGCCTCA GTGAGCGAGC |
| 4441 | GAGCGCGCAG. |

In particular embodiments of the methods of producing a recombinant AAV vector disclosed herein, the exogenous sequence comprises a sequence encoding an ATP Binding Cassette, Subfamily Member 4 (ABCA4) protein or a portion thereof. In some embodiments, the exogenous sequence comprises a 5′ sequence encoding an ABCA4 protein or a portion thereof. In some embodiments, the exogenous sequence comprises a 3′ sequence encoding an ABCA4 protein or a portion thereof. In some embodiments, the exogenous sequence further comprises a sequence encoding a promoter. In some embodiments, the exogenous sequence comprises a sequence encoding a rhodopsin kinase (RK) promoter. In certain embodiments, the RK promoter is a GRK1 promoter. In some embodiments, the sequence encoding the GRK1 promoter comprises or consists of:

| (SEQ ID NO: 75) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In certain embodiments, the exogenous sequence comprises a sequence encoding a chicken beta-actin (CBA) promoter. In some embodiments, the sequence encoding the CBA promoter comprises or consists of:

| (SEQ ID NO: 76) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |
| or | |
| (SEQ ID NO: 77) | |
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

In some embodiments, the sequence encoding the ABCA4 is a human ABCA4 sequence or a variant thereof. In certain embodiments, the sequence encoding ABCA4 comprises a 5′ nucleotide sequence comprising nucleotides 1-3701 or 1-4326 of SEQ ID NO: 2 or SEQ ID NO: 1. In certain embodiments, the sequence encoding ABCA4 comprises a 3′ nucleotide sequence comprising nucleotides 3154-6822, 3196-6822, 3494-6822, 3603-6822, 3653-6822, 3678-6822, 3702-6822 or 3494-6822 of SEQ ID NO: 2 or SEQ ID NO: 1. In particular embodiments, the methods disclosed herein are used to produce upstream and/or downstream ABCA4 vectors that may be used according to a dual vector system disclosed herein. In particular embodiments, the ABCA4 vectors include, but are not limited to, those disclosed in or comprising sequences disclosed in any of FIGS. 307-335.

In particular embodiments of the methods of producing a recombinant AAV vector disclosed herein, the plasmid vector comprising an exogenous sequence, the helper plasmid vector or the plasmid vector comprising the sequence encoding a viral Rep protein and a viral Cap protein further comprises a sequence encoding a selection marker.

In particular embodiments of the methods of producing a recombinant AAV vector disclosed herein, the sequence encoding the viral Rep protein and the sequence encoding the viral Cap protein comprise sequences isolated or derived from AAV serotype 8 (AAV8) viral Rep protein and viral Cap protein sequences, including variants thereof.

### BRIEF DESCRIPTION OF THE DRAWINGS

The file of this patent contains at least one drawing/photograph executed in color. Copies of this patent with color drawings(s)/photographs(s) will be provided by the Office upon request and payment of the necessary fee.

Several of the drawings are chromatograms. Generally, the green line indicates fluorescence, the red line indicated absorbance (260 nm), the blue line indicates absorbance (280 nm), and the black line indicates conductibity. Viewed in black and white, the conductivity line typically starts low and increases over time, and the absorbance (260 nm) and absorbance (280 nm) lines largely track each other.

FIG. 1 is a diagram summarizing exemplary cell culture and expansion steps of the manufacturing process. Cells in serum containing adherent cell culture are passaged and expanded through the steps shown to populate twenty HYPERstacks (36 layered culture vessel).

FIG. 2 is a schematic overview of AAV8-RPGR upstream manufacturing process including in-process limits and QC testing.

FIG. 3 is a schematic flow diagram of the cell thaw step.

FIG. 4 is a table showing the parameters and operating ranges/setpoints for the cell thaw process.

FIG. 5 is a table showing key materials/consumables used in the cell thaw process.

FIG. 6 is a schematic flow diagram of the generic passage procedure.

FIG. 7 is a table showing generic guidance for the cell passage regime.

FIG. 8 is a table showing recommended reagent volumes (HBSS, cell dissociation solution and growth media) and cell seeding densities for cell passages.

FIG. 9 is a table showing key materials/consumables used in the cell thaw and passage regimes.

FIG. 10 is a diagram summarizing the transfection and harvesting steps of the manufacturing process. Cells are transfected using a polyethylenimine (PEI) based transfection protocol. (1) DNA and PEIpro® are diluted separately in Transfection Solution. (2) The PEI solution is added dropwise to the DNA solution and incubated for 10 minutes at room temperature. (3) The DNA/PEI solution is added to the previously prepared Transfection Media (DMEM+4 mM stabilized glutamine or stabilized glutamine dipeptide+10% FBS). (4) Growth Media (DMEM+4 mM stabilized glutamine or stabilized glutamine dipeptide+10% FBS) is removed from the HYPERstack, Transfection Media containing DNA/PEI is added and cells are incubated at 37° C., 5% CO2 for 24 hours. (5) The Transfection Media is removed from the HYPERstack, Harvest Media (DMEM+4 mM stabilized glutamine or stabilized glutamine dipeptide+0% FBS+Benzonase) is added and cells are incubated at 37° C., 5% CO2 for 72 hours. (6) Virus Release Solution is added to the HYPERstack and cells are incubated in, 5% CO2 for 18 hours to release AAV particles.

FIG. 11 is a table showing a guide to creating the calcium phosphate mediated transfection solution per 5×36-layer HYPERStacks®.

FIG. 12 is a table showing a guide to creating the PEIpro® mediated transfection solution per 5×36-layer HYPERStacks®.

FIG. 13 is a schematic flow diagram of the transient transfection and media harvest steps.

FIG. 14 is a table showing the volumes of chloroquine and media required for the initial media change, as a function of the production scale.

FIG. 15 is a table showing the parameters and operating ranges/setpoints for the transfection and harvest steps.

FIG. 16 is a table showing key materials/consumables used in the calcium phosphate cell transfection process.

FIG. 17 is a table showing the key materials/consumables used in the PEI cell transfection process.

FIG. 18 is a schematic flow diagram of the filtration clarification step.

FIG. 19 is a table showing the parameters and operating ranges/setpoints for the clarification filtration step.

FIG. 20 is a table showing the key materials/consumables used in the clarification filtration step.

FIG. 21 is a diagram summarizing the downstream processing steps (DSP) of the manufacturing process. (1) Harvest Media containing AAV particles is collected from the HYPERstack. (2) Diluted Harvest media is purified by Hydrophobic Interaction Chromatography (HIC), the peak containing AAV particles is selected, and the eluate collected. (3) Diluted HIC eluate is further purified by cation exchange chromatography (CEX), the peak containing rAAV particles is selected and the eluate collected. (5) Diluted CEX eluate is enriched for full rAAV particles by anion exchange chromatography (AEX) with gradient elution, the peak containing full rAAV particles is selected and the eluate collected. (6) AEX eluate is concentrated and diafiltrated into final formulation buffer (FFB) (without Pluronic F-68) via two tangential flow filtration (TFF) steps using a 100 kDa hollow fiber filter (HFF). (7) Pluronic F-68 (also referred to as poloxamer 188) is added and the drug substance is frozen at −80° C.

FIG. 22 is a schematic overview of the AAV8-RPGR downstream and fill and finish manufacturing process including QC testing and in-process controls.

FIG. 23A is a table showing the advantages of macro-porous chromatography technology.

FIG. 23B is a series of 3 images of chromatography media showing, from left to right, a membrane, a monolith and a conventional bead.

FIGS. 24A and B are a pair of graphs depicting HPLC analytics on initial material (Fingerprint, Total particles, Empty/Full particles) (Harvest) (left graph depicts partial separation method analysis and right graph depicts total analysis).

FIG. 25 is a photographs of an SDS-PAGE analysis of rAAV-RPGR harvest material

FIG. 26 is an exemplary result for total host DNA and protein from samples of harvested media and harvested media post-clarification.

FIG. 27 is a table summarizing the hydrophobic interaction chromatography (HIC) AAV capture process.

FIG. 28 is a schematic diagram depicting stability testing procedures for hydrophobic conditions.

FIG. 29 is a graph depicting a chromatogram from HIC procedure outlined in FIG. 89.

FIG. 30 is a table depicting the results of HIC at harvest, before filtration (BF), and after filtration (AF) by measuring OD600, conditions as depicted in FIG. 89.

FIG. 31 is a table depicting the running conditions of HIC without filtration of load.

FIG. 32 is a pair of chromatographs corresponding to the HIC running conditions of FIG. 31.

FIG. 33 is a pair of photographs depicting SDS-PAGE analyses of the HIC depicted in FIG. 31 and FIG. 32.

FIG. 34 is a schematic diagram depicting HIC with potassium phosphate (KP) precipitation. Results in less protein denaturation and higher protein stability (native).

FIG. 35 is a pair of chromatograms corresponding to the HIC experiment of FIG. 34 (using a C4 A column).

FIG. 36 is a pair of chromatograms corresponding to the HIC experiment of FIG. 34 (using an OH column).

FIG. 37 is a series of photographs depicting SDS-PAGE analyses of the HIC depicted in FIG. 95-97.

FIG. 38 is a series of tables depicting results of (NH4)2SO4 and PK using C4 A and OH columns.

FIG. 39 is a schematic diagram depicting HIC conditions—loading amount in this figure is loading amount for FIGS. 40-96 and 102.

FIG. 40 is a pair of chromatograms corresponding to the HIC experiment of FIG. 39.

FIG. 41 is a schematic diagram depicting loading capacity of HIC on 1 mL column, for example, as shown in FIGS. 39 and 40.

FIG. 42 is a pair of chromatograms depicting the FLD response of the HPLC total Analytics of the initial material.

FIG. 43 is a pair of ddPCR analyses (a table and chromatograph for each) for two HIC experiments. HIC-9 was performed without sorbitol. HIC-10 was performed using sorbitol.

FIG. 44 is a pair of tables depicting ddPCR analyses for two HIC experiments. HIC-10 was performed on an OH column. HIC-10 was performed on a C4 A column.

FIG. 45 is a pair of graphs showing a comparison of linear gradient elution and the optimized step elution for the HIC purification step

FIG. 46 is a series of chromatograms depicting robustness of HIC experiments by comparison of molarity of HIC dilution buffer.

FIG. 47 is a series of chromatograms depicting capacity of HIC experiments on a 2 mL column (HIC-16 and HIC-17).

FIG. 48 is a table depicting chromatographic conditions for HIC-18.

FIG. 49 is a pair of chromatograms corresponding to FIG. 48.

FIG. 50 is a pair of tables and a chromatogram depicting ddPCR results from HIC-18 (run on an OH 80-mL capacity column).

FIG. 51 is a table depicting chromatographic conditions for HIC-19.

FIG. 52 is a pair of chromatograms corresponding to FIG. 51.

FIG. 53 is a pair of tables and a chromatogram depicting ddPCR results from HIC-19 (run with a step elution).

FIG. 54 is a pair of tables providing conductivity measurements for HIC-18 and HIC-19, respectively, and a chromatogram corresponding to the HIC experiments of FIGS. 49, 50, and 51.

FIG. 55 is a table depicting chromatographic conditions for HIC-20.

FIG. 56 is a pair of chromatograms corresponding to FIG. 55.

FIG. 57 is a pair of tables and a chromatogram depicting ddPCR results from HIC-20 (run on an OH 80-mL capacity column).

FIG. 58 is a photograph of a SDS-PAGE analysis of the HIC-20 corresponding to FIG. 57.

FIG. 59 is a table summarizing the type of column, buffer used, and purpose of each of 20 HIC experiments.

FIG. 60A-B are a chromatogram and an SDS-PAGE gel, respectively, which show an exemplary HIC AAV capture step. FIG. 60A shows a chromatogram from an 80 mL column. The HIC capture step has been successfully scaled up from a 1 mL column to an 80 mL column. FIG. 60B shows an SDS-PAGE gel analysis of the HIC Harvest Media, Flow through, Load and eluate fractions. The lanes show, from left to right: marker, input Harvest media, Load, flow through (FT), W, fractions E1, E2, E2 diluted two-fold (E2.2×), E3, diluted two-fold (E3.2×), clean in place (CIP), and clean in place diluted two-fold (CEP.2×). The E2 fraction containing AAV particles is boxed in green, the Harvest Media lane is boxed in red.

FIG. 61A-B are a pair of chromatograms showing a gradient (FIG. 61A) and isocratic elution (FIG. 61B) protocols for the HIC step. E1, E2 and E3 fractions are boxed.

FIG. 62A-B are a pair of SDS-PAGE gels showing the rational for a 2 versus a 3 step process. FIG. 62A shows an exemplary HIC elution. FIG. 62B shows an AEX full to empty separation proof of concept run. The fraction containing capsids is boxed in red (FIG. 62A, while the fraction containing empty and full capsids after the AEX step are boxed in red (left) and green (right) (FIG. 62B). The purity over the HIC step and the subsequent purity of a HIC and AEX QA purified product is not sufficient. The intermediate polishing step (CEX cation exchange, SO₃−) is required.

FIG. 63 is a graph showing the optimization of the filtration step that is after the HIC capture step. On the X-axis are shown different types of filters: PES=polyethersulfone, CA=cellulose acetate, GF=glass fibre, PVDF=polydivinyl fluoride, PTFE=polytetrafluoroethylene, MV=mixed esters, RC=regenerated cellulose. On the y axis are shown the average recovery of AAV particles (%) for each filter type. Orange bars indicate filters with limited scale up options (PVDF and PTFE).

FIG. 64A-B are a chromatogram and an SDS-PAGE gel, respectively, showing the capture of rAAV particles using hydrophobic interaction chromatography (HIC). In FIG. 16A, absorbance in mAU is indicated on the y-axis from 0 to 300 in increments of 50. Fractions E2 and E3 containing rAAV particles are boxed in dark green and light green, respectively. Wash, eluate, and CIP fractions are indicated on the X axis. FIG. 64B is an SDS-PAGE gel showing the purity of the eluted fractions from FIG. 64A. The lanes showing Fraction E2 containing rAAV particles are boxed. 2× indicates two-fold dilution.

FIG. 65A-B are a chromatogram and a table, respectively, showing step recoveries of an exemplary HIC step.

FIG. 66A-B are a chromatogram and three images of transmission electron microscopy (TEM) micrographs, respectively, showing AAV particles purified using HIC. FIG. 66A is a chromatogram showing the elution of AAV particles purified in an exemplary HIC step. Fractions E3, E4 and E5 containing AAV particles are indicated with brackets on the x axis. FIG. 66B shows TEM micrographs of the AAV particles eluted in the E3, E4 and E5 fractions. Scale bars indicate 200 nm.

FIG. 67 is a series of six TEM micrographs of the E3, E4 and E5 HIC fractions at two different magnifications. In the top row, scale bars, from left to right, indicate 0.5 μM, 0.5 μM, and 500 nM. IN the bottom row, scale bars indicate 200 nm.

FIG. 68 is a table summarizing the cation exchange chromatography (CEX) process for AAV intermediate purification.

FIG. 69 is a pair of chromatograms depicting a development intermediate purification step SO3 performed at either pH 4.0 (SO3-1) or pH 3.5 (SO3-2).

FIG. 70 is a photograph of an SDS-PAGE analysis of the intermediate purification SO3 step performed at pH 3.5 (SO3-2).

FIG. 71 is a pair of tables and a chromatogram depicting ddPCR results for SO3-2.

FIG. 72 is a table depicting chromatographic conditions for SO3-3.

FIG. 73 is a pair of chromatograms corresponding to FIG. 72.

FIG. 74 is a pair of tables and a chromatogram depicting ddPCR results for SO3-3.

FIG. 75 is a table depicting chromatographic conditions for SO3-4.

FIG. 76 is a pair of chromatograms corresponding to FIG. 75.

FIG. 77 is a photograph of an SDS-PAGE analysis of SO3-4.

FIG. 78 is a pair of chromatograms depicting an intermediate purification step SO3 performed at either pH 3.8 (SO3-5) or pH 3.6 (SO3-7).

FIG. 79 is a photograph of an SDS-PAGE analysis showing that pH 3.6±0.1 is a preferred or optimal pH for HIC experiments using conditions of FIGS. 69-78.

FIG. 80 is an analysis of column capacity determination on SO3.

FIG. 81 is a table depicting chromatographic conditions for SO3-9, capacity run without filtration of load material.

FIG. 82 is a pair of chromatograms corresponding to FIG. 135.

FIG. 83 is a table depicting chromatographic conditions for SO3-10, capacity run with filtration of load material.

FIG. 84 is a pair of chromatograms corresponding to FIG. 135.

FIG. 85 is a series of chromatograms comparing SO3-7, SO3-9 and SO3-10.

FIG. 86 is a pair of tables depicting HPLC analytics for SO3-9 and SO3-10.

FIG. 87 is a table depicting chromatographic conditions for SO3-11.

FIG. 88 is a chromatogram corresponding to FIG. 141.

FIG. 89 is a pair of ddPCR analyses for either without poloxamer, SO3-7 (left graph and chromatogram) or with poloxamer SO3-11 (right graph and chromatogram).

FIG. 90 is a table depicting chromatographic conditions for SO3-12.

FIG. 91 is a photograph showing the SO3-12 Load sample and the SO3-12 FT sample.

FIG. 92 is a pair of chromatograms corresponding to FIG. 90.

FIG. 93 is a pair of ddPCR analyses for either HIC-20 (left graph and chromatogram) or SO3-12 (right graph and chromatogram).

FIG. 94 is a photograph of an SDS-PAGE analysis of SO3-12.

FIG. 95 is a table summarizing the type of column, buffer used, and purpose of each of 12 SO3 experiments.

FIG. 96 is a HPLC chromatogram determining the Full:Empty ratio of the material following intermediate purification SO3-12.

FIG. 97A-B are a chromatogram and an SDS-PAGE gel, respectively, that show an intermediate polishing step by CEX using an SO3− column matrix. FIG. 97A shows a pH 3.6 SO3− zoomed in chromatogram, with the fraction containing rAAV particles boxed. FIG. 97B shows an SDS-PAGE gel of the pH 3.5 (E2), pH 3.6 (SO3 7 E2), pH 3.8 (SO3-5 E2) and pH 4.0 (E2) samples. All gels were slightly overdeveloped in order to expose all protein bands in the present sample. There are slightly less contaminants present in the lower pH samples than in the samples with higher pH. The optimal pH is 3.6+/−0.1.

FIG. 98A-D are a pair of chromatograms (FIG. 98A, C) and a pair of SDS-PAGE gels corresponding to the chromatograms (FIG. 98B, D), showing pH optimization of the CEX step. FIG. 98A, B are at pH 4.0, FIG. 98C, D are at pH 3.5.

FIG. 99A-C are a series of 2 transmission electron micrographs (FIG. 99A-B) and a table (FIG. 99C) showing a transmission electron microscopic (TEM) analysis of the SO3 CEX eluate. In the sample, 21.8% of AAVs were neither full nor empty. Blue arrows indicate full capsid AAVs, red arrows indicate empty capsid AAVs, and green arrows indicate uncertain (neither full nor empty) AAVs.

FIG. 100A-B are a chromatogram and an SDS page gel, respectively, showing the elution of AAV particles CEX in the AAV intermediate (polishing) purification step. In FIG. 100A, the y-axis shows absorbance in mAU, indicated from 0 to 2500 in increments of 500. Wash, eluate and CIP fractions are indicated on the x axis. Fractions E2 and E3 containing AAV particles are boxed in dark green and light green, respectively. FIG. 100B is an SDS-PAGE gel showing the purity of the eluted fractions from FIG. 100A. The lanes showing fraction E2 containing AAV particles are boxed. 2× and 10× indicate two-fold and ten-fold dilutions, respectively.

FIG. 101 is a table summarizing the anion exchange chromatography (AEX) process for enrichment of rAAV full particles.

FIG. 102 is a HPLC chromatogram depicting the QA elution profile of material following intermediate purification (SO3-12) using different pH of buffers without MgCl₂.

FIG. 103A-B are a chromatogram and a heat plot, respectively, showing the resolution of full and empty peaks as a function of pH and MgCl₂ concentration. FIG. 103A shows overlaid AEX QA matrix chromatograms (A260 signal) at pH 9.5 with varying concentrations of MgCl₂. The black arrow indicates 0 mM MgCl₂, the orange arrow indicates 2 mM MgCl₂, the blue arrow indicates 1 mM MgCl₂. FIG. 103A is a heat plot illustrating the ability to separate full and empty particles, with pH on one axis and MgCl₂ on the other. Separation is indicated by color from minimum (purple) to maximum (white). Optimal separation is seen at pH 9.0 and 0 mM MgCl₂.

FIG. 104A-B are a chromatogram and an SDS-PAGE gel, respectively, showing the enrichment of full AAV particles using AEX. In FIG. 104A, the y-axis shows absorbance in mAU, indicated from 0 to 100 in increments of 50. Fractions E2, E3, E4, E5 and E6 are indicated on the X axis. Fraction E3 containing full AAV particles is boxed. FIG. 104B is an SDS-PAGE gel showing the purity of the eluted fractions from FIG. 104A. Fraction QA2 E3 containing full rAAV particles is boxed.

FIG. 105A-F are two chromatograms (FIG. 105A, D), three tables (FIG. 105B, C, F) and an SDS-PAGE gel (FIG. 105E) summarizing the full particle enrichment step. FIG. 105A is an exemplary AEX QA-2 chromatogram, while FIG. 105D is a zoom of the chromatogram in FIG. 105A. FIG. 105B is a table summarizing the full particle purity estimation by spectrophotometry. An A260:A280 ratio of about 1.3 as seen in the E3 fraction indicates a high percentage of full particles. FIG. 105C is a table summarizing the full particle content estimation by HPLC of the QA2 E2 and E3 AEX fractions. FIG. 105E is an SDS-PAGE gel showing the QA2 AEX load, eluate and CIP fractions. Fraction E3 containing full AAV particles is boxed. FIG. 105F is a table summarizing full particle recovery in each fraction by HPLC.

FIG. 106A-C are a TEM micrograph, and two tables, respectively, showing the enrichment of full AAV particles by anion exchange chromatography (AEX). FIG. 106A is a TEM micrograph of the QA2 E3 fraction showing rAAV particles. Scale bar indicates 200 nm. FIG. 106B shows the titer of AAV particles by Droplet Digital PCR (ddPCR). The E3 fraction is indicated with a green box. FIG. 106C shows the number of counted viruses, the percent of full and partial particles by percentage, and the estimated number of empty/damaged particles by percentage for fraction AQ2E3 (also referred to as QA2 E3).

FIG. 107 is a table showing the expected yields at each step of the manufacturing process.

FIG. 108A-D is a series of graphs showing ddPCR results for samples S03-14 E1, QA-3 (A), QA-4 (B), QA-5 (C), and QA-6 (D).

FIG. 109 is a chart providing TEM results for QA-3 through QA-8. All samples were clear, without impurities, aggregates of particles were rarely noticed in samples SO3-14, QA-3 E3, QA-6 E3 and QA-8 E3. Ratio between full and empty/damaged viruses were similar in all QA samples (71-77%), but was lower in SO3-14 sample (46%). Some of the particles were not classified as full or empty. A third group of viruses was introduced (unclassified). Viruses from this group were not electron lucent on the whole surface, but displayed just electron dense spot on the surface. Such viruses could be full, not completely full, not correctly formed or damaged.

FIG. 110 is a chromatogram and corresponding table showing comparison of purification of empty and full particles under QA-7 (capacity) and QA-8 (regular conditions).

FIG. 111 is a pair of chromatogram showing purification of QA-8. Lower chromatogram is a higher magnification of the upper chromatogram.

FIG. 112A-C is a series of tables providing ddPCR and HPLC E/F results. Preparative runs from QA-7 onwards were performed using analytical column (QA-0.1 mL with 2 μm pores).

FIG. 113 is a pair of SDS-page analyses showing presence of protein found at each step of purification for each of QA-7 and QA-8.

FIG. 114 is a pair of TEM micrographs and a corresponding table showing the full fraction (E3) from run QA-8.

FIG. 115 is a table providing chromatographic conditions for S03 15.

FIG. 116 is a pair of chromatograms showing purification of S0315. The bottom chromatogram is a higher magnification of the top chromatogram.

FIG. 117A-B is a pair of tables providing HPLC (A) and ddPCR (B) results for SO3 15.

FIG. 118 is a table providing chromatographic conditions for QA-9.

FIG. 119 is a pair of chromatograms showing purification using QA-9. The bottom chromatogram is a higher magnification of the top chromatogram.

FIG. 120 is a table providing chromatographic conditions for QA-10.

FIG. 121 is a pair of chromatograms showing purification using QA-10. The bottom chromatogram is a higher magnification of the top chromatogram.

FIG. 122 is a table providing chromatographic conditions for QA-11.

FIG. 123 is a pair of chromatograms showing purification using QA-11. The bottom chromatogram is a higher magnification of the top chromatogram.

FIG. 124 is a table providing chromatographic conditions for QA-12.

FIG. 125 is a chromatogram showing purification using QA-12.

FIG. 126 is a pair of chromatograms showing empty/full ratio using QA-9. The bottom chromatogram is a higher magnification of the top chromatogram.

FIG. 127 is a chromatogram showing empty/full ratio using QA-9.

FIG. 128 is a pair of chromatograms showing empty/full ratio using QA-10. The bottom chromatogram is a higher magnification of the top chromatogram.

FIG. 129 is a chromatogram showing empty/full ratio using QA-10.

FIG. 130 is a pair of chromatograms showing empty/full ratio using QA-11. The bottom chromatogram is a higher magnification of the top chromatogram.

FIG. 131 is a chromatogram showing empty/full ratio using QA-11.

FIG. 132A-C is a series of tables providing ddPCR and HPLC results from QA-9, QA-10 and QA-11.

FIG. 132D is a table providing the empty/full ratio, purity, and recovery from QA-9, QA-10 and QA-11.

FIG. 133 is a table providing elution properties from preparative runs QA-9, QA-10 and QA-11.

FIG. 134 is a series of SDS-page analyses showing protein purifications using preparative runs QA-9, QA-10 and QA-11.

FIG. 135 is a table providing virus count, percent full, percent empty and percent unclassified following purification and TEM analysis of purified viruses from S03-15 E1, QA-9, QA-10 and QA-11. All samples contained small aggregates, which were composed mostly of damaged or not completely formed viruses. Ratio between full and empty/damaged viruses were similar in QA-10 and QA-11 samples (74%), but was lower in SO3-14 sample (45%) and higher in sample QA-9 E3. Some of the particles were not classified as full or empty. A third group of viruses was introduced (unclassified). Viruses from this group were not electron lucent on the whole surface, but displayed just electron dense spot on the surface. Such viruses could be full, not completely full, not correctly formed or damaged.

FIG. 136 is a table providing chromatographic conditions for QA-13.

FIG. 137 is pair of a chromatograms of QA-13 elucidating fractionation method. The bottom chromatogram is a higher magnification of the top chromatogram.

FIG. 138 is a table providing conditions for TFF exchange into formulation buffer.

FIG. 139 is series of chromatograms showing HPLC E/F coupled with MALS detector analytics.

FIG. 140 is series of chromatograms showing HPLC E/F coupled with MALS detector analytics.

FIG. 141 is a table summarizing the empty/full ratios, purity and recovery percentages for each step of virus purification using QA-13.

FIG. 142 is a table summarizing the composition of each of samples S03-14, QA-3, QA-4, QA-5, QA-6, and QA-8 (relevant for FIGS. 142-156). Five samples of Adeno associated virus (AAV) and one additional sample for analysis with transmission electron microscopy (TEM) were analysed to determine viral integrity and to evaluate the relation between full/empty particles.

FIG. 143 is a TEM micrograph showing viruses were spread evenly throughout the grid (S03-14) when observed under low magnification. For FIGS. 143-170, samples were prepared for examination with TEM using negative staining method. Thawed samples were mixed gently and applied on freshly glow-discharged copper grids (400 mesh, formvar-carbon coated) for 5 minutes, washed and stained with 1 droplet of 1% (w/v) water solution of uranyl acetate. Two grids were prepared for each sample. The grids were observed with transmission electron microscope Philips CM 100 (FEI, The Netherlands), operating at 80 kV. At least 10 grid squares were examined thoroughly and a lot of micrographs (camera ORIUS SC 200, Gatan, Inc.) were taken to evaluate the relation between full and empty particles. Micrographs were taken coincidentally at different places on the grid.

FIG. 144 is a pair of representative micrographs of sample SO3-14; small aggregates were present (black arrow). Impurities were not detected and only a few small aggregates could be noticed.

FIG. 145 is a micrograph showing particles which could not be classified neither as full nor as empty/damaged (white arrows).

FIG. 146 is a pair of micrographs showing that in sample QA3-E3 more aggregates were present in comparison to sample SO3-14 and aggregates could be slightly larger. Other impurities could not be found.

FIG. 147 is a pair of micrographs showing empty/damaged particles marked with black arrow and non-classified marked with white arrow. Non-classified particles could represent full virus, but they did not looked perfect.

FIG. 148 is a micrograph showing that viruses (QA-4 E3) were evenly spread throughout the grid. No impurities or aggregates were found.

FIG. 149 is a pair of representative micrograph of QA-4 E3 showing full, empty and non-classified particles.

FIG. 150 is a pair of representative micrographs of QA-5 E3 showing full, empty and non-classified particles. No impurities or aggregates were found. Empty/damaged particles marked with black arrow and non-classified marked with white arrow. Non-classified particles could represent full virus, but they did not looked perfect.

FIG. 151 representative micrograph of QA-5 E3 showing full, empty and non-classified particles under low magnification.

FIG. 152 is a pair of representative micrograph of QA-6 E3 showing full, empty and non-classified particles. No impurities or aggregates were found. Viruses were spread evenly (left micrograph); a few aggregates were present (right micrograph).

FIG. 153 is a pair of representative micrographs of sample QA-6 E3 chosen for evaluation full/empty ratio; empty/damaged particles were marked with black arrows, non-classified with white arrow.

FIG. 154 is a micrograph of QA-8 E3 viruses observed under low magnification. Sample was without impurities, but contained some small aggregates.

FIG. 155 is a pair of representative micrographs of sample QA-8 E3; small aggregate (black arrow) contains damaged viruses.

FIG. 156 is a table providing a ratio between full and empty/damaged particles. The ratio between full and empty/damaged viruses was determined by counting the particles in selected micrographs taken at the same magnification. Sample SO3-14 contained 46% of full viruses, all other samples contained higher and more similar % of full viruses (71-77%). All samples were clear, without impurities, aggregates of particles were rarely noticed in samples SO3-14, QA-3 E3, QA-6 E3 and QA-8 E3. Ratio between full and empty/damaged viruses were similar in all QA samples (71-77%), but was lower in SO3-14 sample (46%).

FIG. 157 is a table providing the compositions of each sample used in the analyses for FIGS. 157-169.

FIG. 158 is a representative TEM micrograph showing S03-15 E1 viruses of non-diluted sample observed under low magnification. Viruses were spread evenly throughout. Samples were prepared for examination with TEM using negative staining method. Thawed samples were mixed gently and applied on freshly glow-discharged copper grids (400 mesh, formvar-carbon coated) for 5 minutes, washed and stained with 1 droplet of 1% (w/v) water solution of uranyl acetate. Three grids were prepared for each sample, one with non-diluted and two with diluted sample. We diluted sample with 0.1 M PB. The grids were observed with transmission electron microscope Philips CM 100 (FEI, The Netherlands), operating at 80 kV. At least 10 grid squares were examined thoroughly and several micrographs (camera ORIUS SC 200, Gatan, Inc.) were taken to evaluate the ratio between full and empty particles. Micrographs were taken coincidentally at different places on the grid.

FIG. 159 is a pair of representative micrographs of sample SO3-15; left: non-diluted sample; right: diluted sample. Viruses were spread evenly throughout the grid.

FIG. 160 is a pair of representative micrographs of sample SO3-15; left: non-diluted sample; right: diluted sample. Viruses were spread evenly throughout the grid, few small aggregates were present in non-diluted, as well as in diluted sample (white arrow).

FIG. 161 is a pair of representative micrographs of QA-9 E3 viruses of non-diluted (left) and diluted (right) sample observed under low magnification. Viruses were evenly spread and just a few aggregates could be found. No other impurities were present.

FIG. 162 is a pair of representative micrographs of QA-9 E3 viruses of non-diluted (left) and diluted (right) sample chosen for counting. Viruses were evenly spread and just a few aggregates could be found. No other impurities were present.

FIG. 163 is a pair of representative micrographs of QA-9 E3 viruses. Most of the viruses were full with characteristic shape (left); small aggregates contained damaged particles (right).

FIG. 164 is a pair of representative micrographs of QA-10 E3 viruses of non-diluted (left) and diluted (right) sample observed under low magnification. All grids with sample QA-10 E3 expressed appropriate quality. Beside some small aggregates we found other structures which might represented completely disintegrated viruses (FIG. 165, right micrograph); such structures were present on all three grids of the sample, but were bound just on small part of the grids. Sample QA-10 E3 contained more damaged particles in comparison to the sample QA-9 E3.

FIG. 165 is a pair of representative micrographs of QA-10 E3 viruses of diluted sample QA-10 E3 with denoted almost completely damaged viruses (left); right micrograph: most probably the rest of destroyed viruses.

FIG. 166 is a representative micrograph of non-diluted sample QA-10 E3 chosen for virus counting. 21 micrographs were used for counting the particles and calculation of ratio between full and empty/damaged viruses.

FIG. 167 is a representative micrograph of QA-11 E3 viruses of non-diluted sample observed under low magnification. Sample QA-11 E3 contained small aggregates. Ratio between full and empty/damaged viruses was determined with counting the particles on 33 micrographs taken at same magnification.

FIG. 168 is a pair of representative micrographs of QA-11 E3 viruses non-diluted (left) and diluted (right) sample chosen for counting.

FIG. 169 is a table providing the ratio between full and empty/damaged particles for each sample. The ratio between full and empty/damaged viruses by counting the particles in selected micrographs taken at the same magnification. Particles were classified into 3 groups: full, unclassified, empty and damaged together. Sample SO3-15 E1 contained 45% of full viruses, sample QA-9 E3 80%, samples QA-10 E3 and QA-11 E3 were similar regarding full/empty ratio (74% of full viruses). All samples contained small aggregates, which were composed mostly of damaged or not completely formed viruses. Ratio between full and empty/damaged viruses were similar in QA-10 and QA-11 samples (74%), but was lower in SO3-14 sample (45%) and higher in sample QA-9 E3. Some of the particles could not be classified as full or empty, thus they were put in a third group as “unclassified”. Viruses from this group were not electron lucent on the whole surface, but displayed just electron dense spot on the surface. Such viruses could be full, not completely full, not correctly formed or damaged.

FIG. 170A-B is a pair of tables providing ddPCR and HPLC results for QA-13 and TFF1 steps.

FIG. 171 is a series of charts and summary table providing HPLC E/F coupled with MALS detector analytics of TFF1.

FIG. 172 is an SDS analysis of purified QA-13 virus.

FIG. 173 is a pair of SDS analyses comparing virus purification following QA and TFF.

FIG. 174 is a schematic overview of AAV8-RPGR upstream manufacturing process including in-process limits and QC testing

FIG. 175 is a schematic flow diagram of the cell thaw step.

FIG. 176 is a table showing recommended minimum warming durations for media warming.

FIG. 177 is a table showing the parameters and operating ranges/setpoints for the cell thaw process.

FIG. 178 is a table showing the materials/consumables used in the cell thaw process.

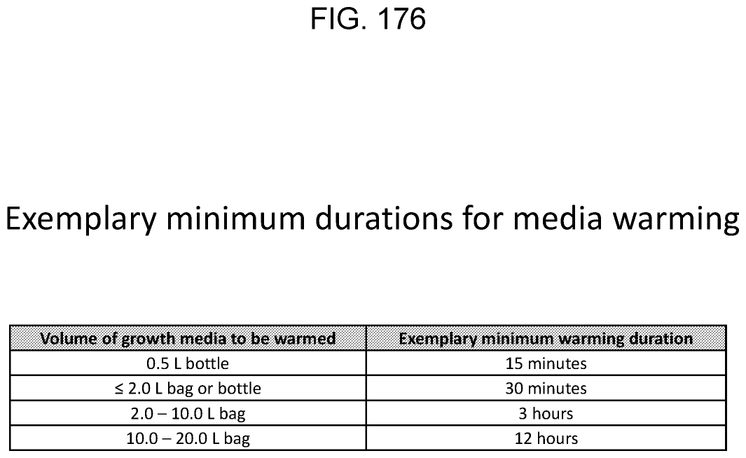

FIG. 176 is a table showing the volumes of chloroquine and media required for the initial media change, as a function of the production scale.

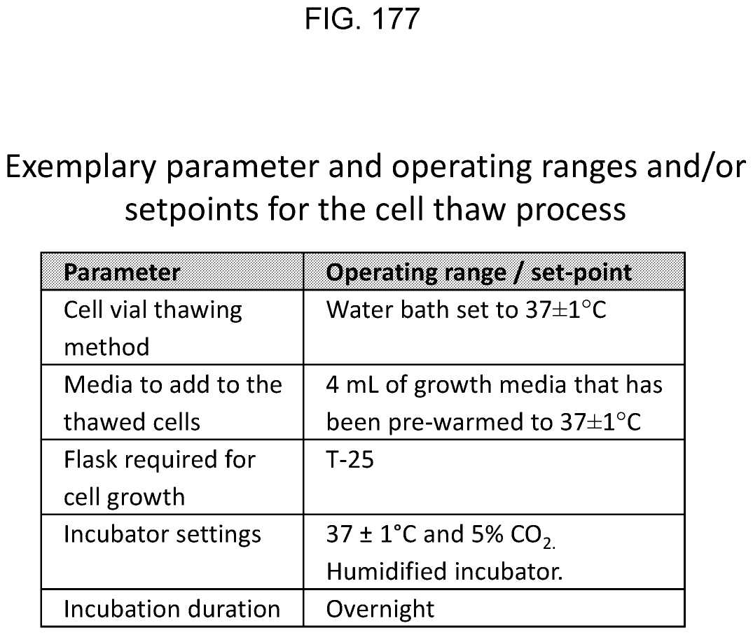

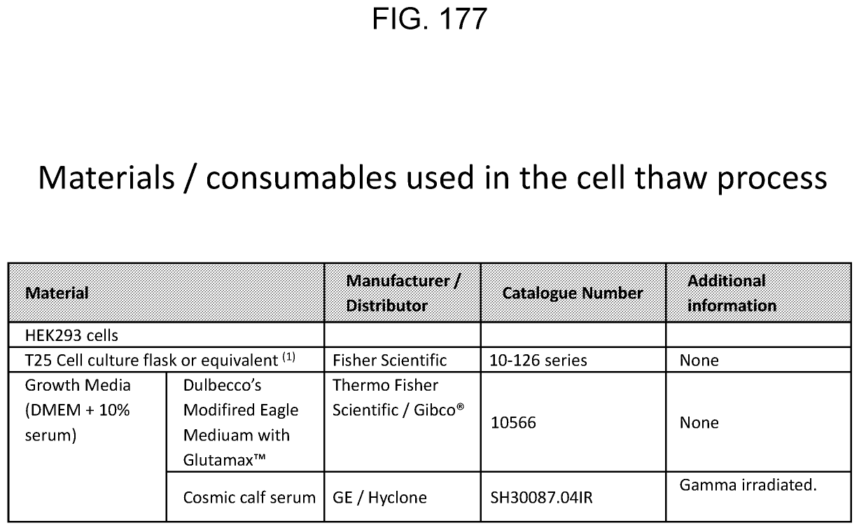

FIG. 177 is a table showing the parameters and operating ranges/setpoints for the transfection and harvest steps.

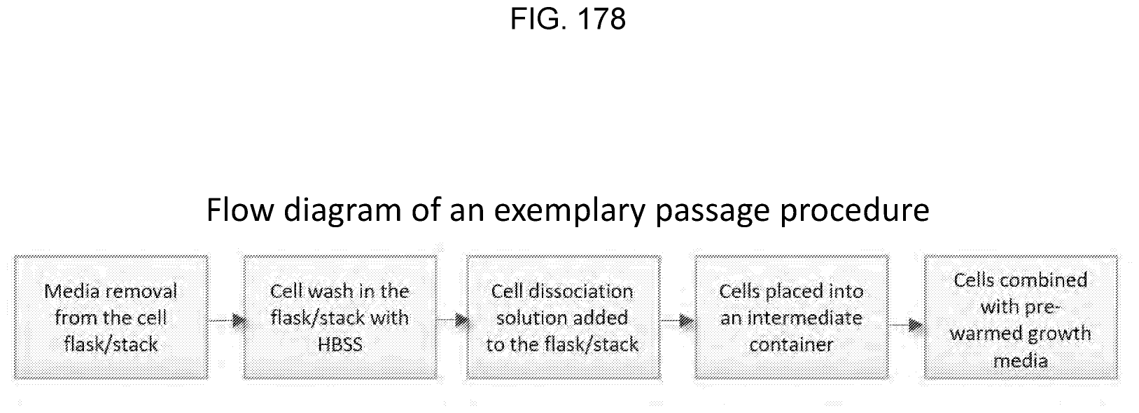

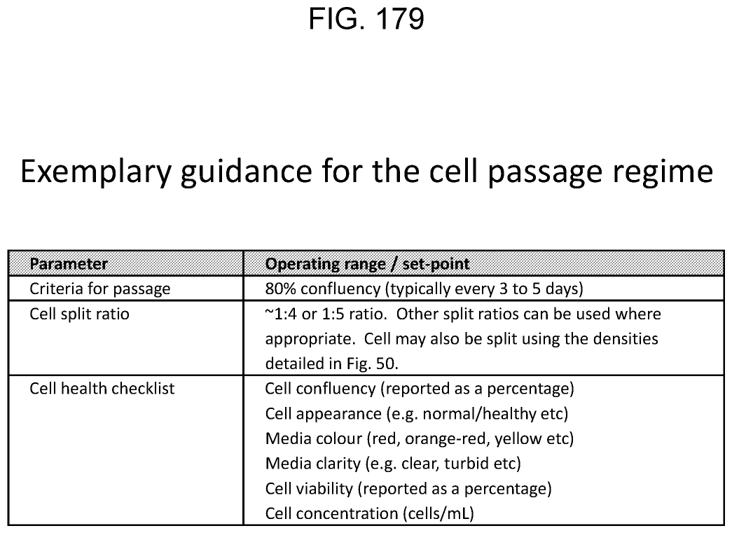

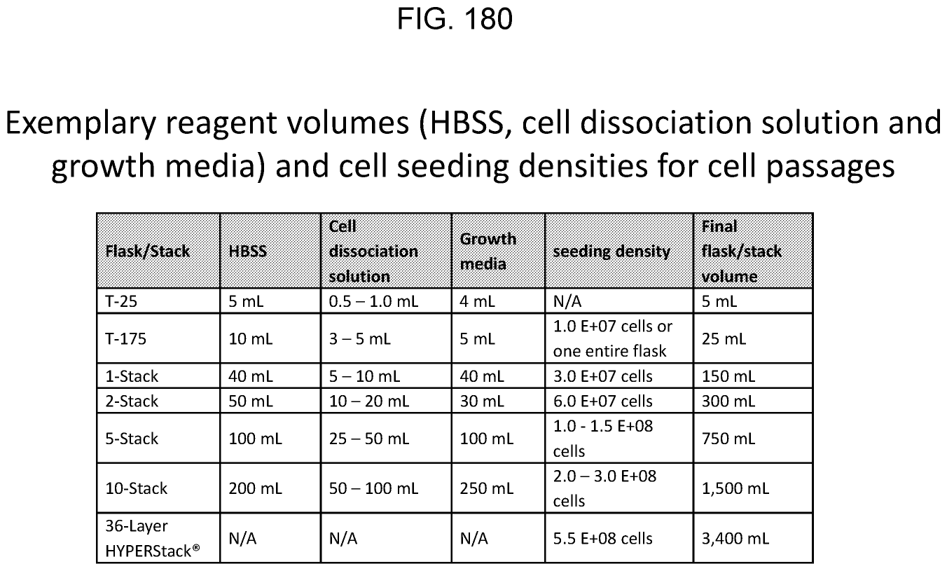

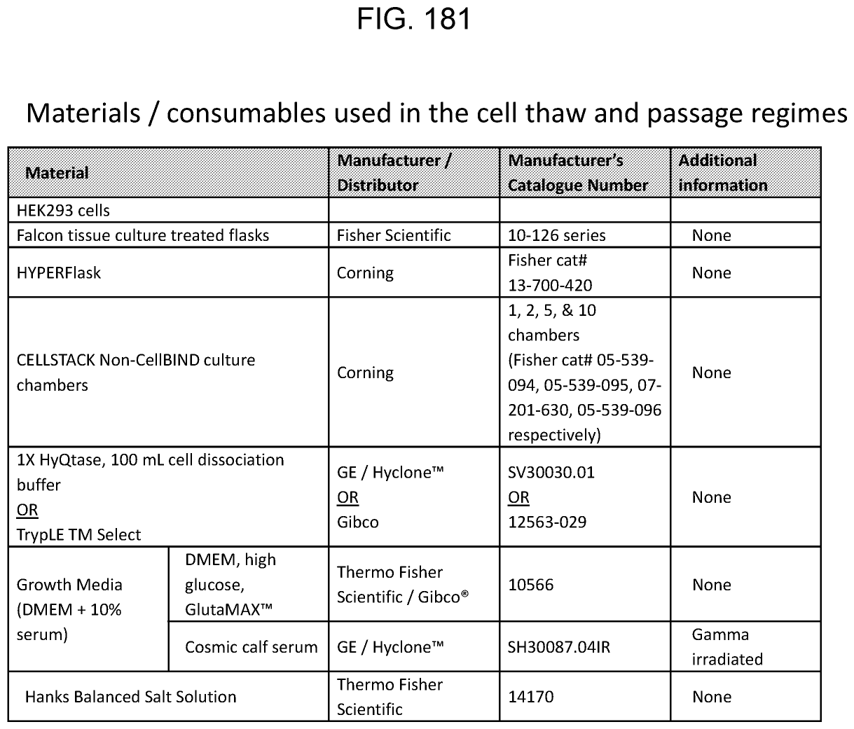

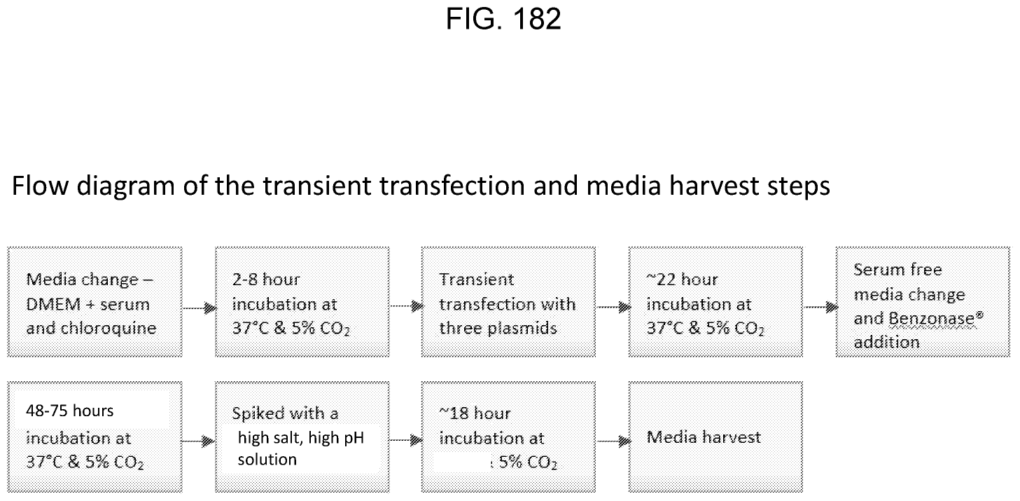

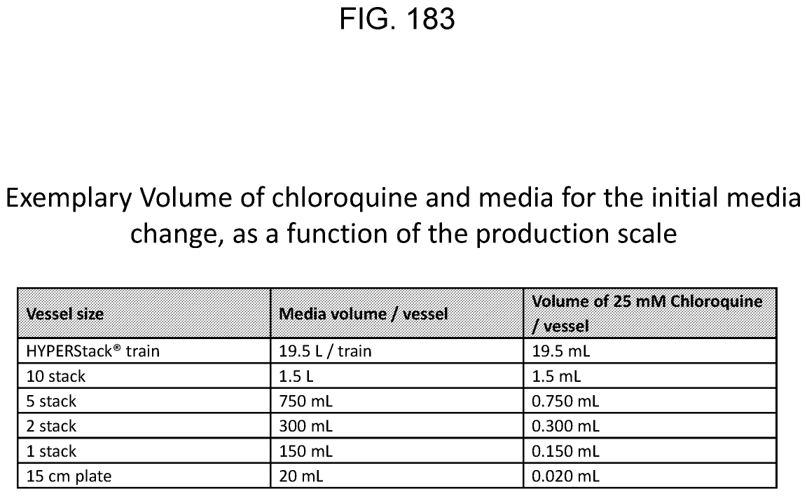

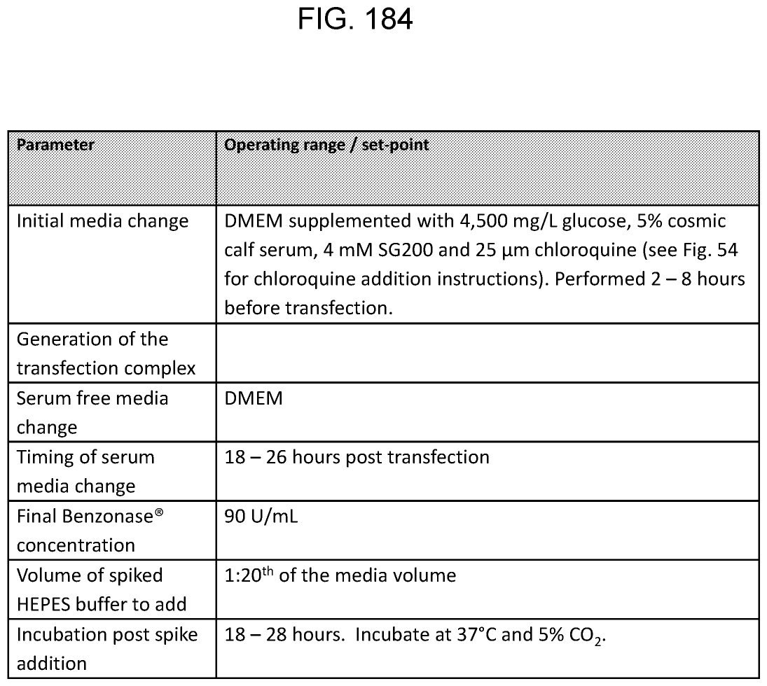

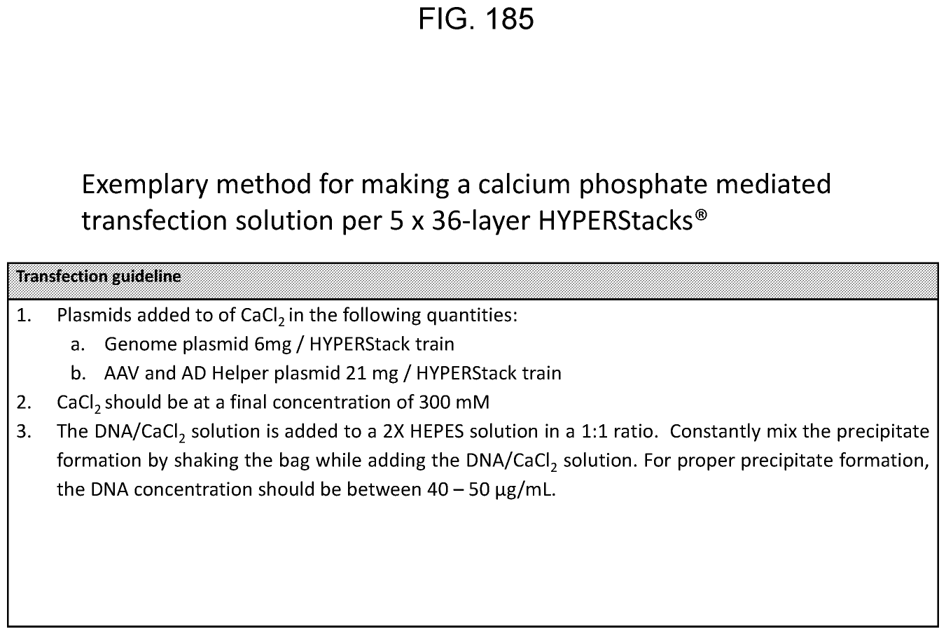

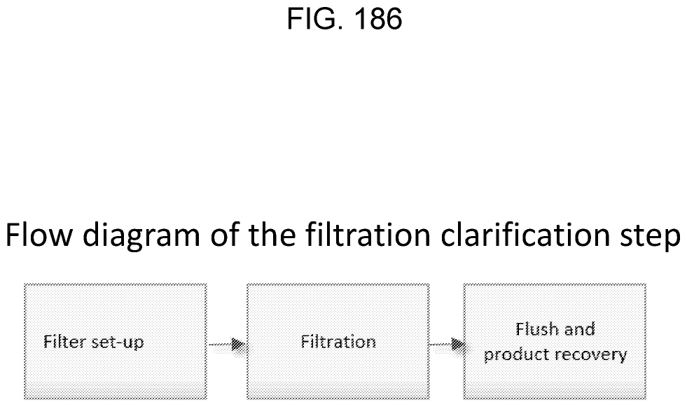

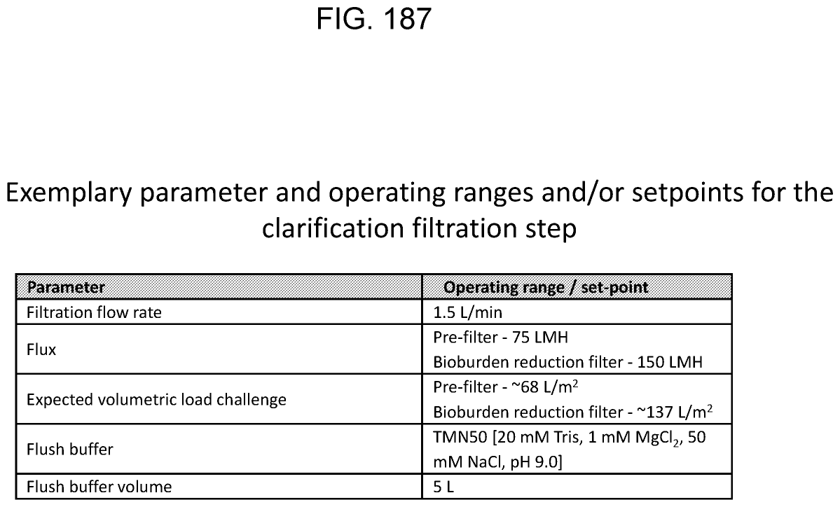

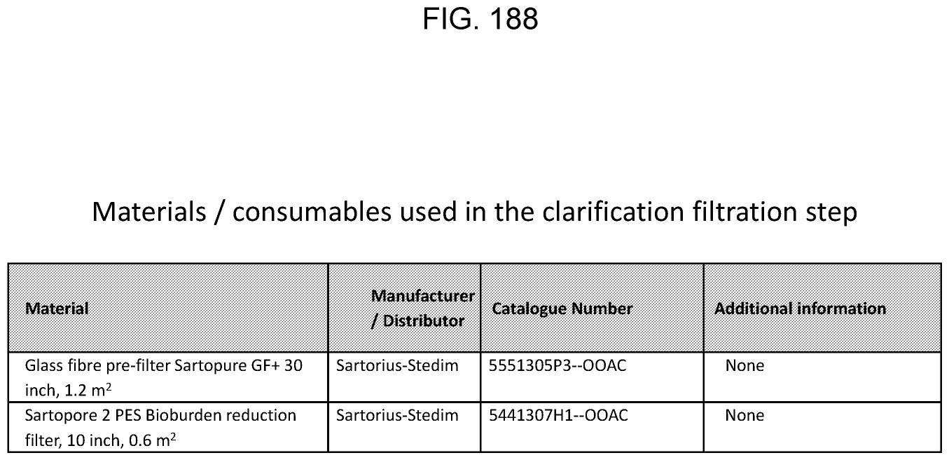

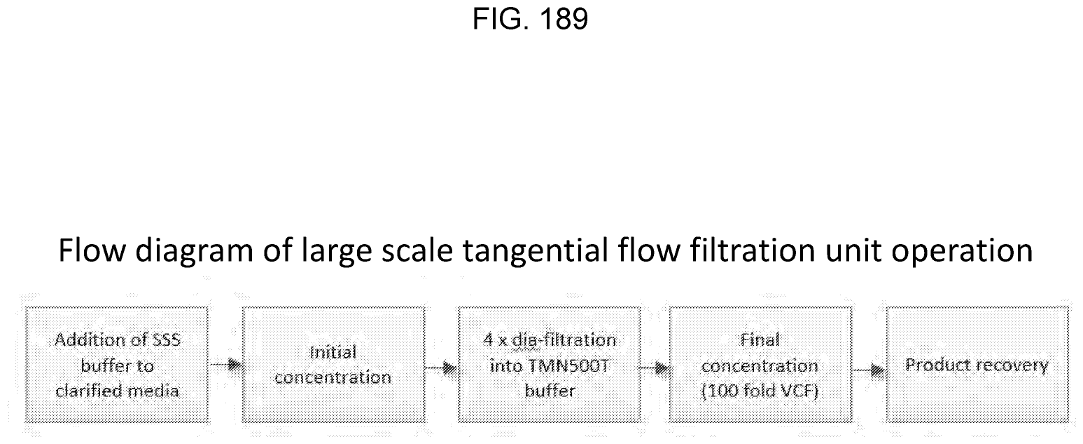

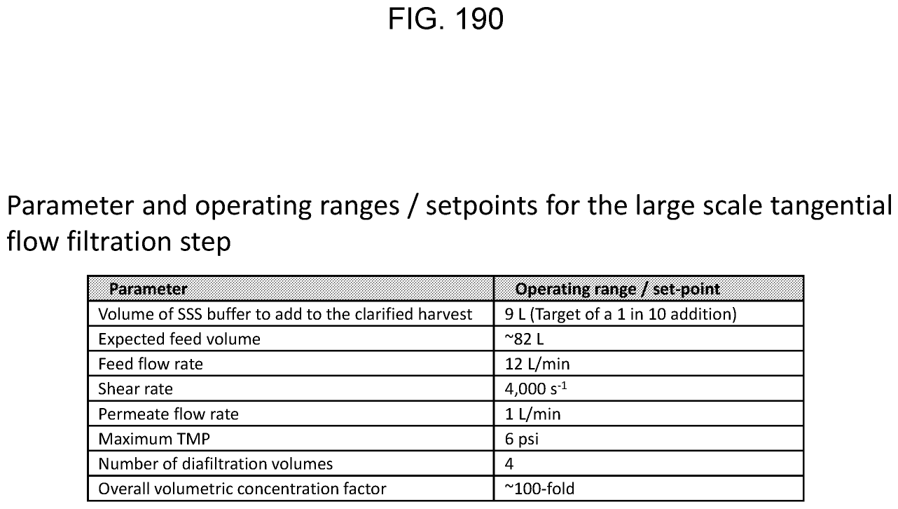

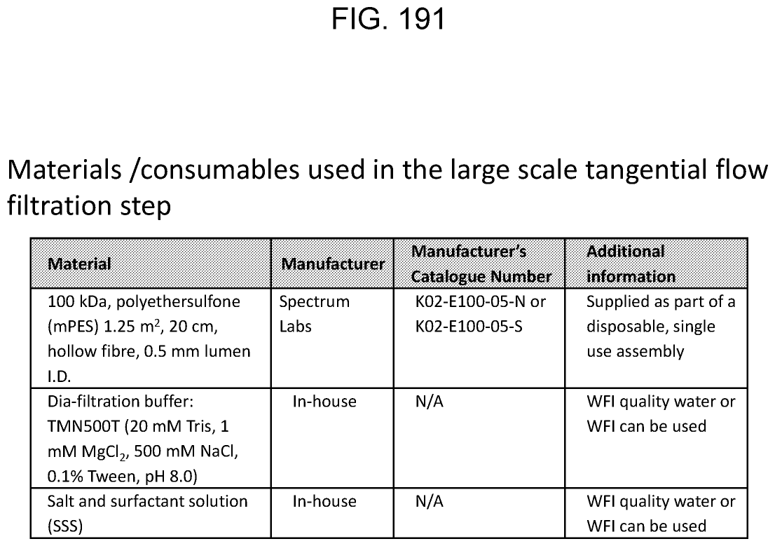

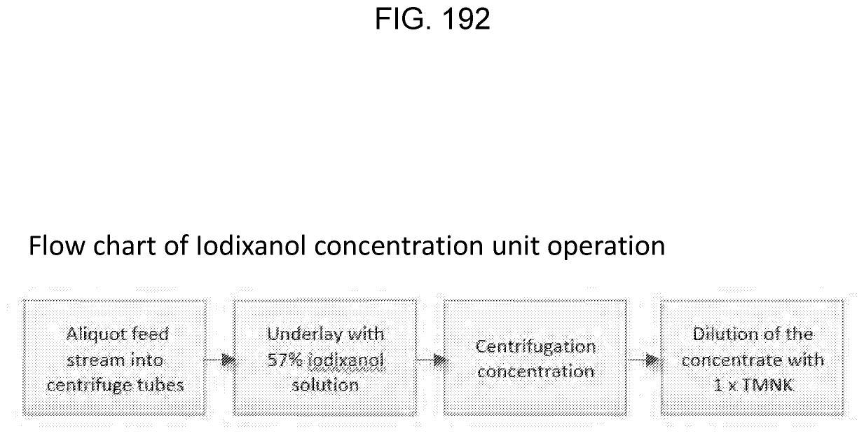

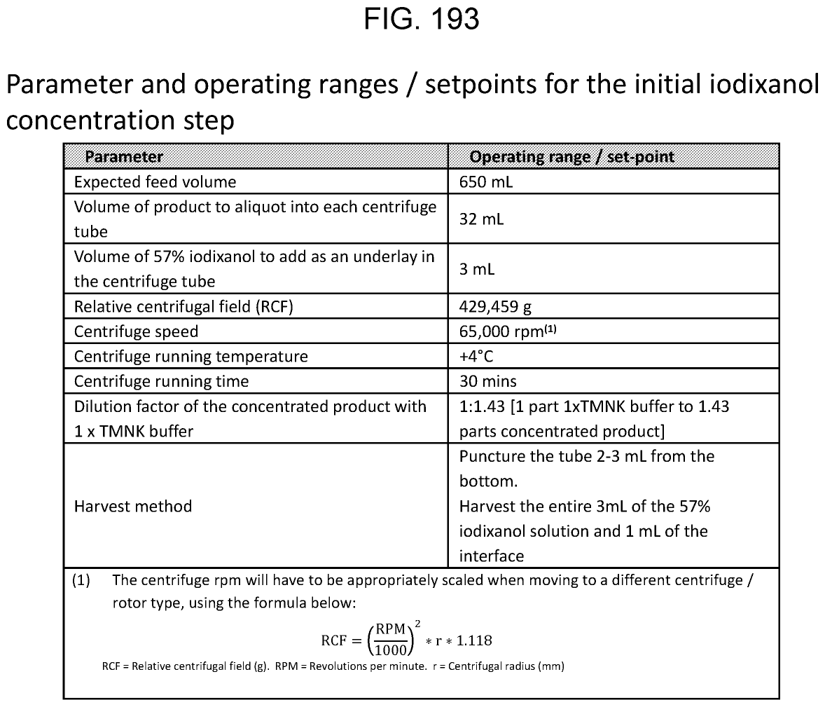

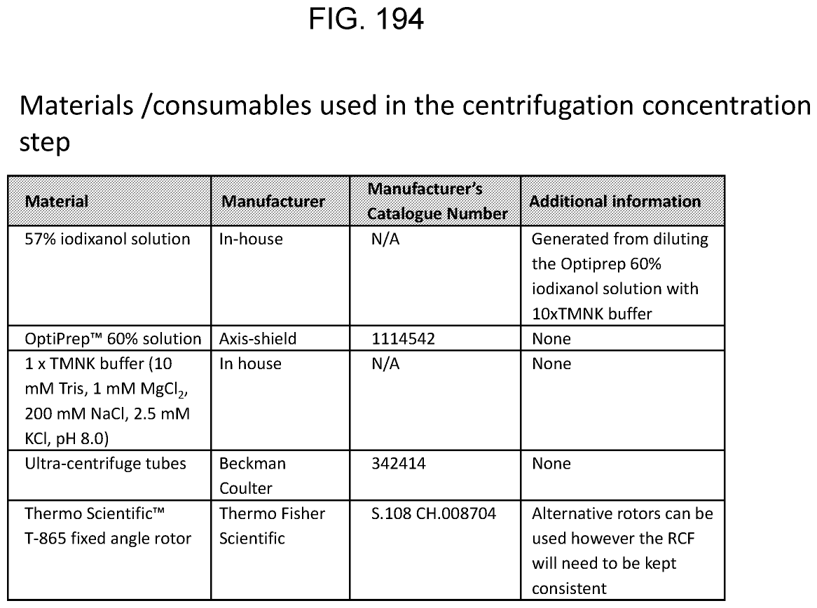

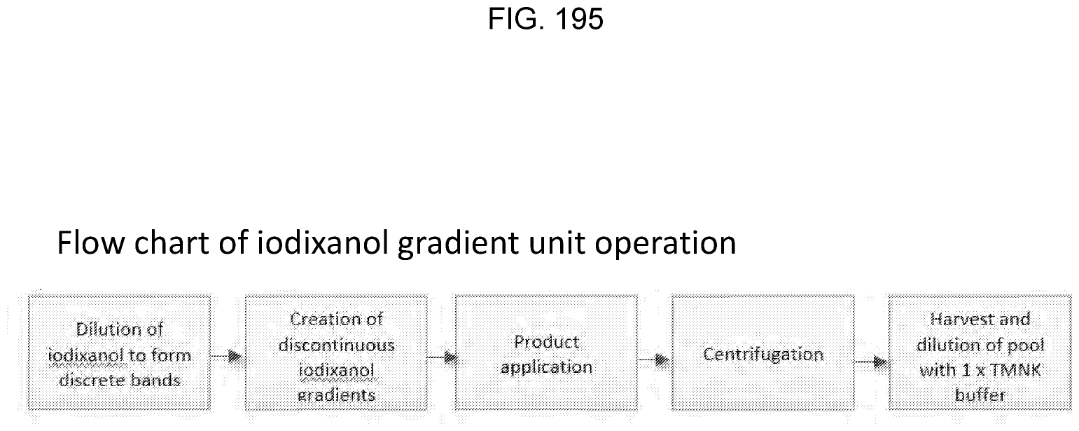

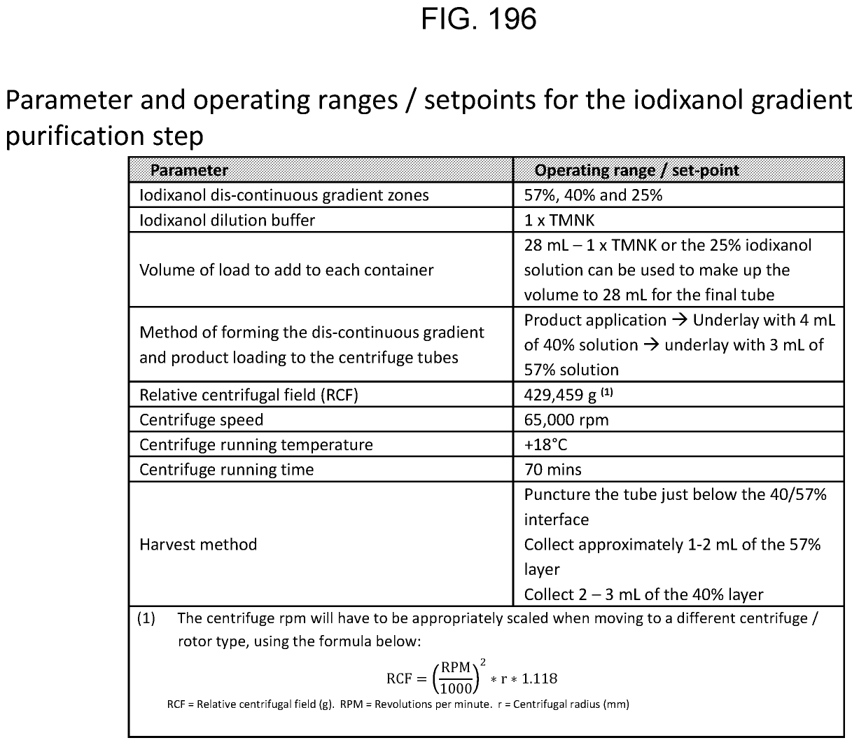

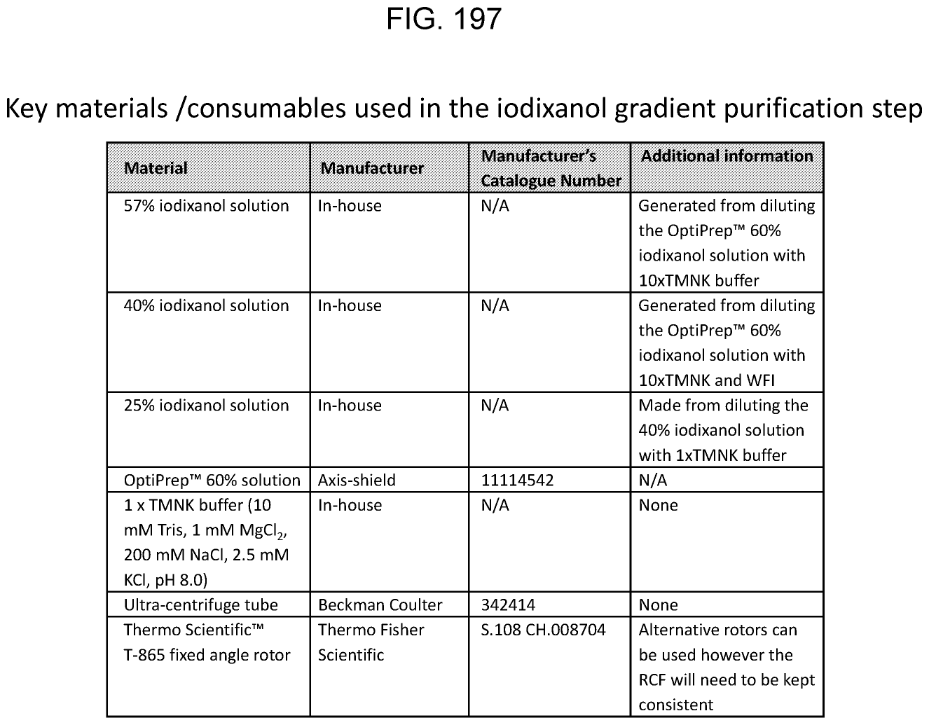

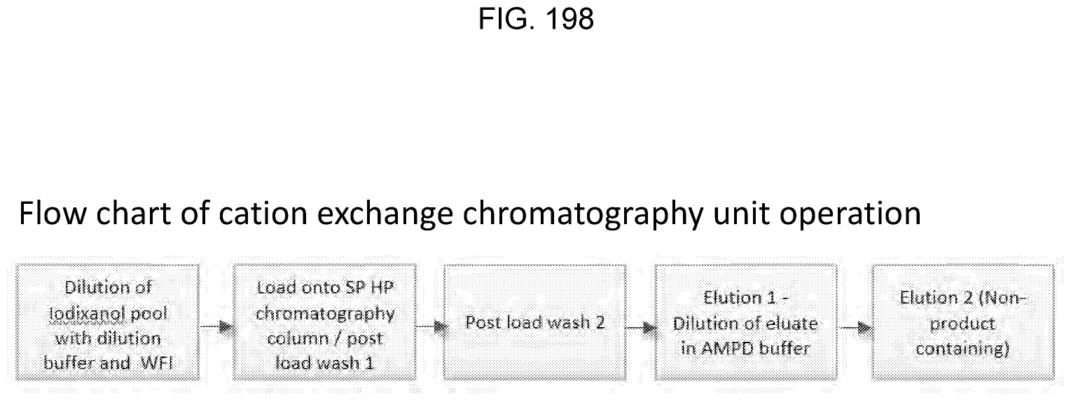

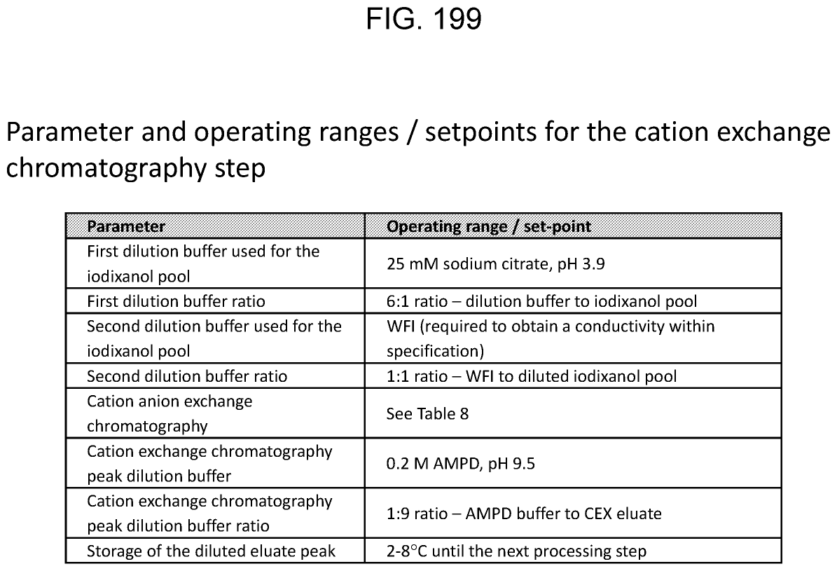

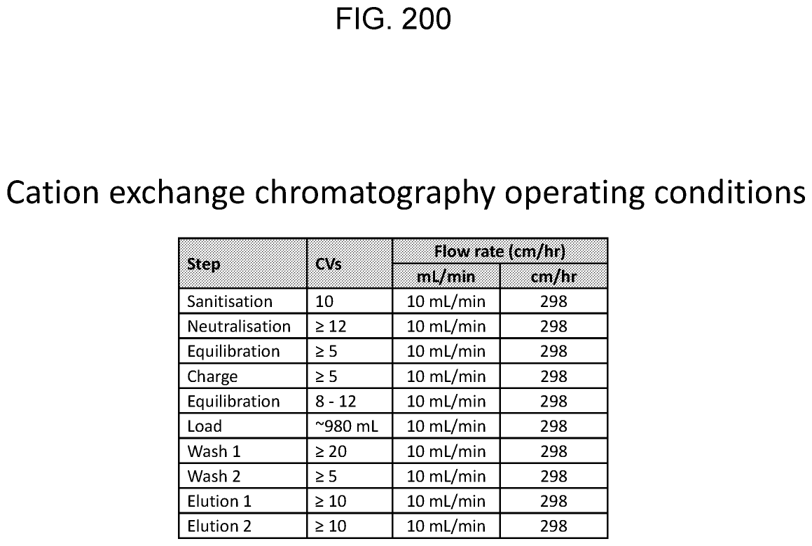

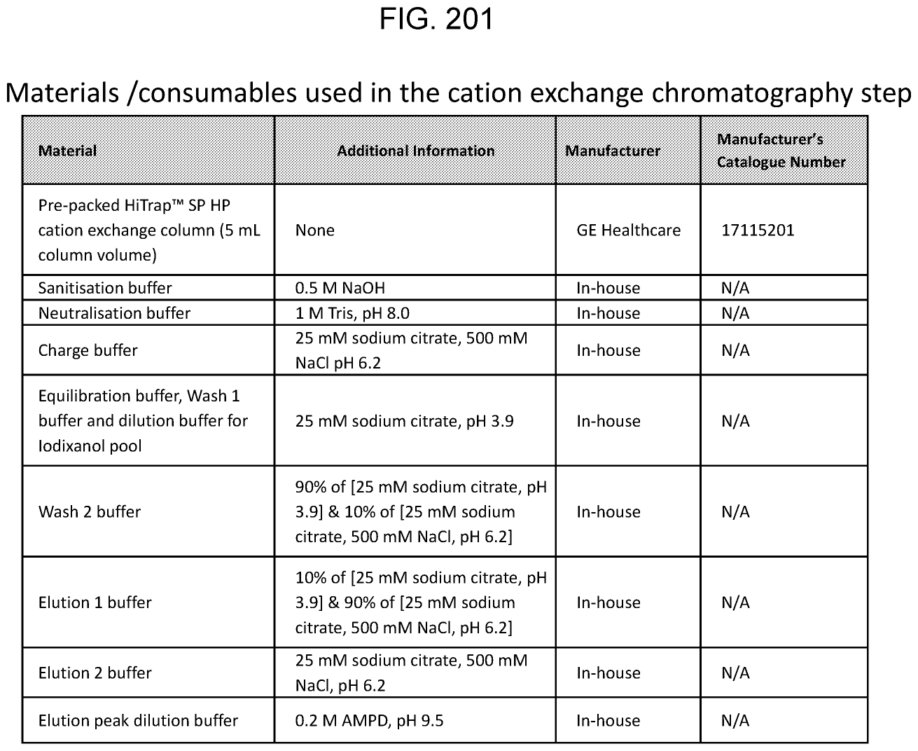

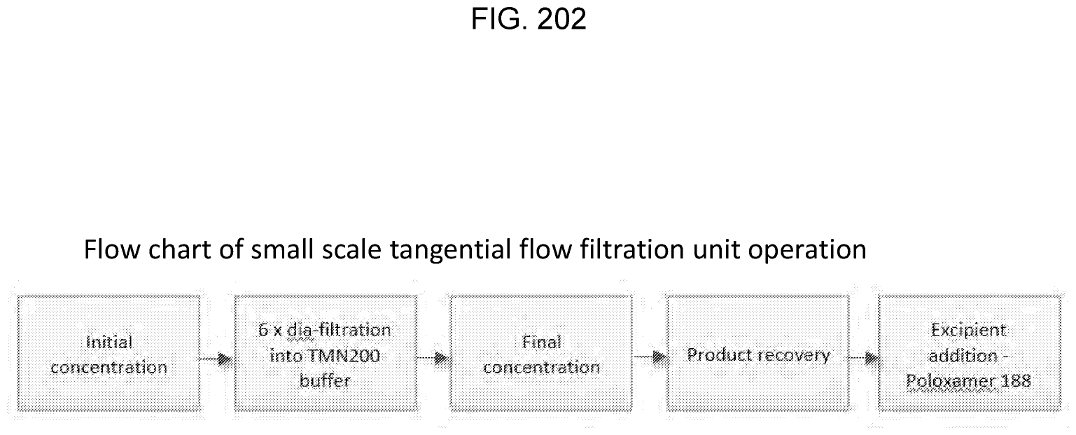

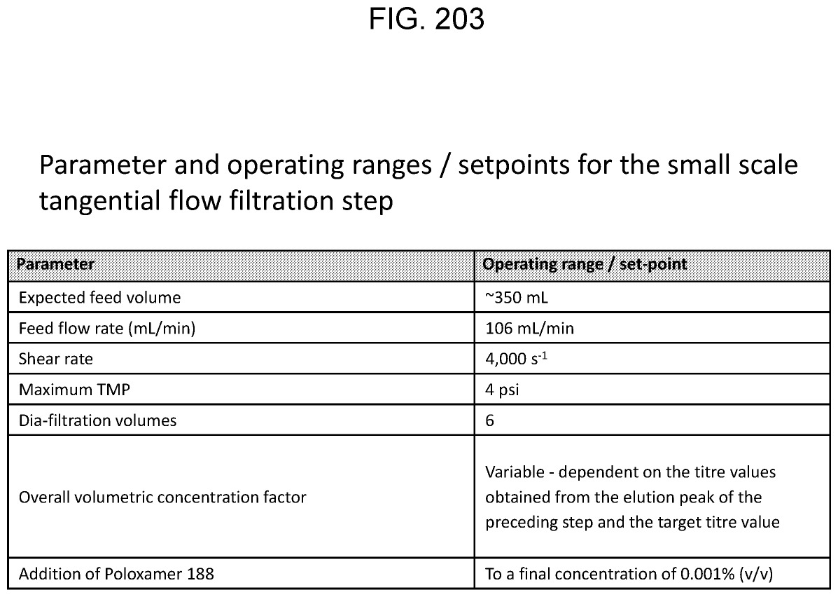

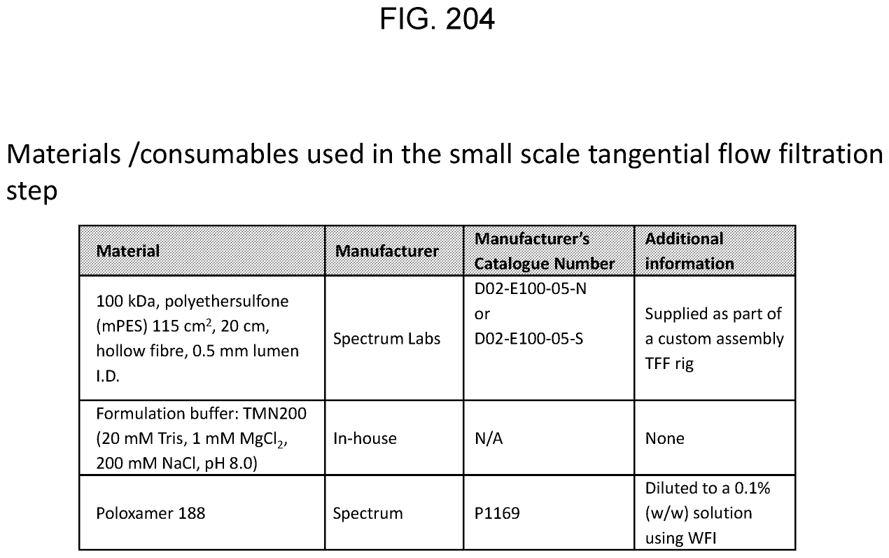

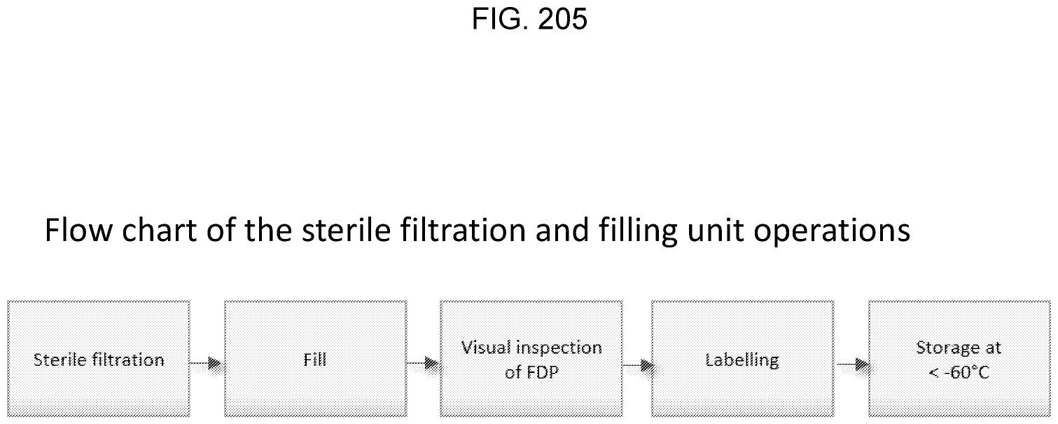

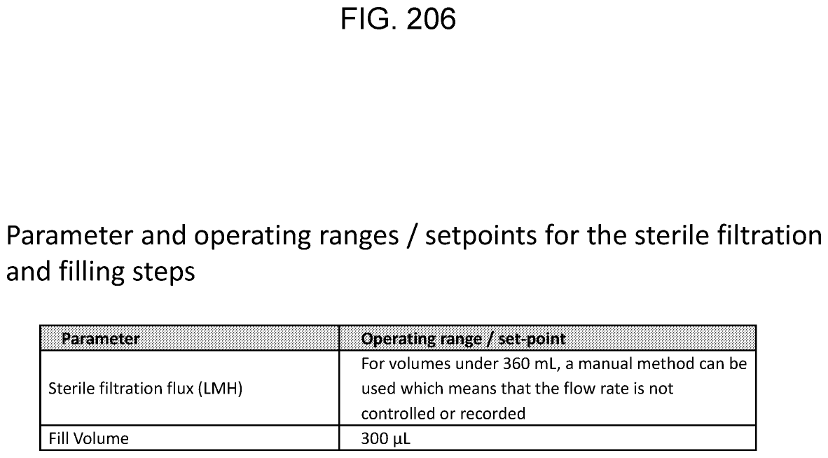

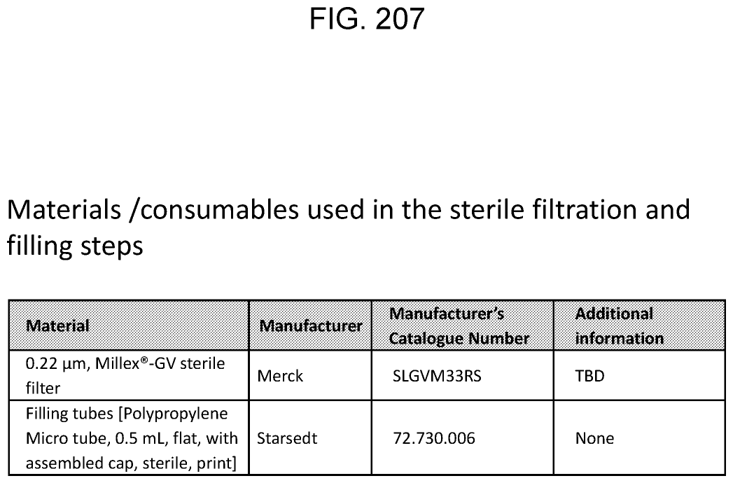

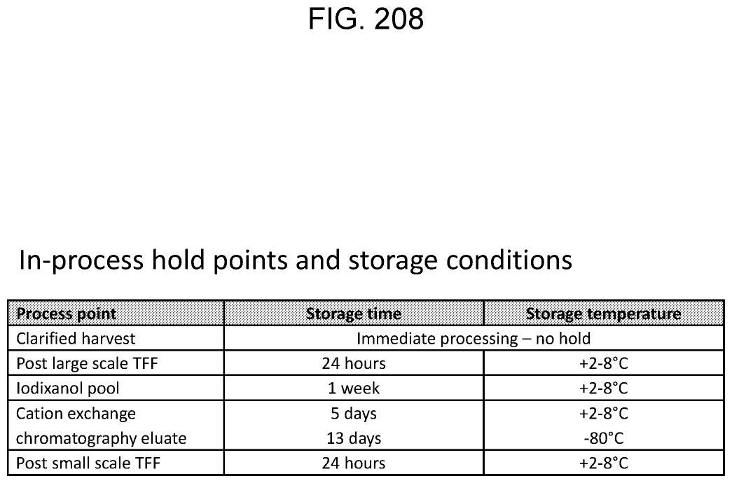

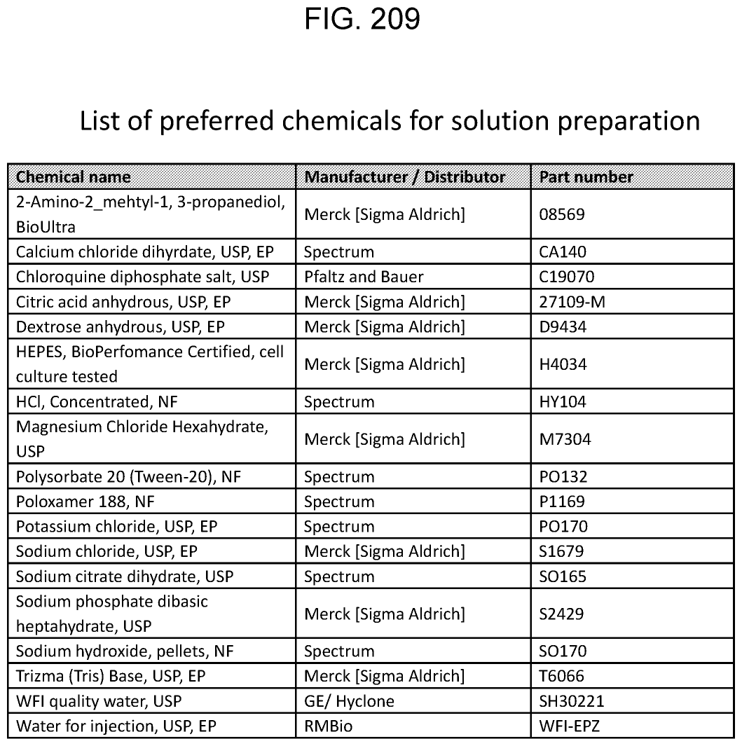

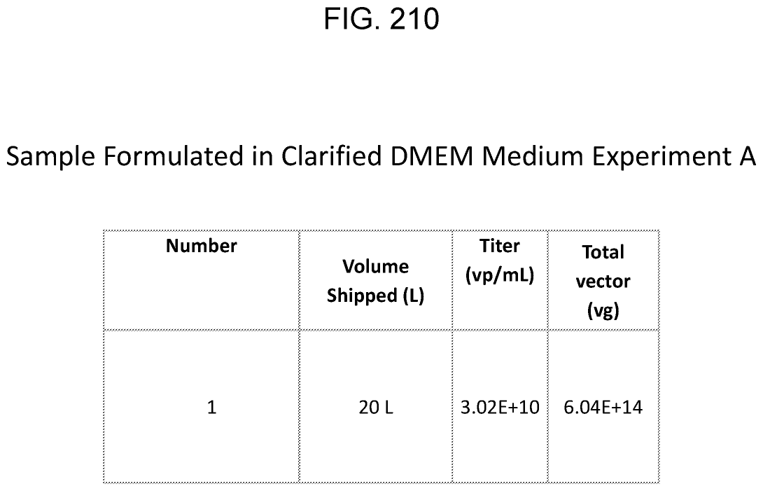

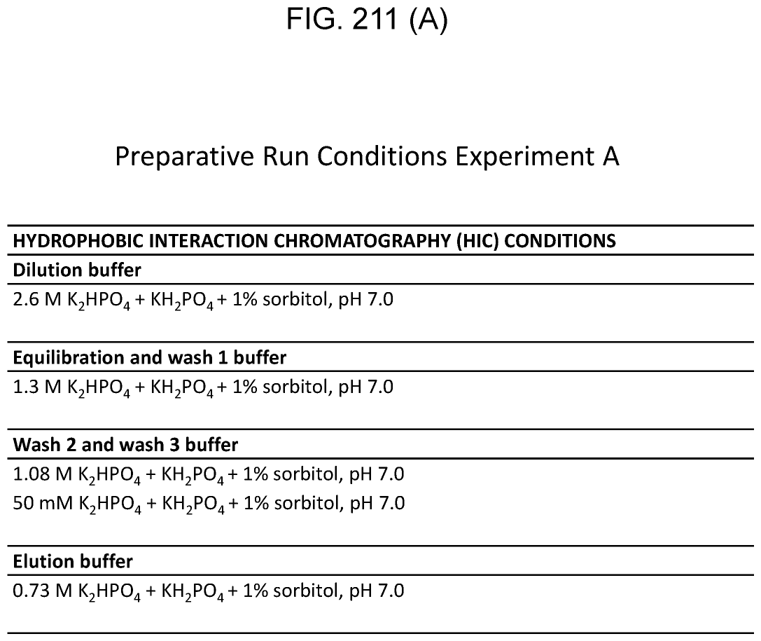

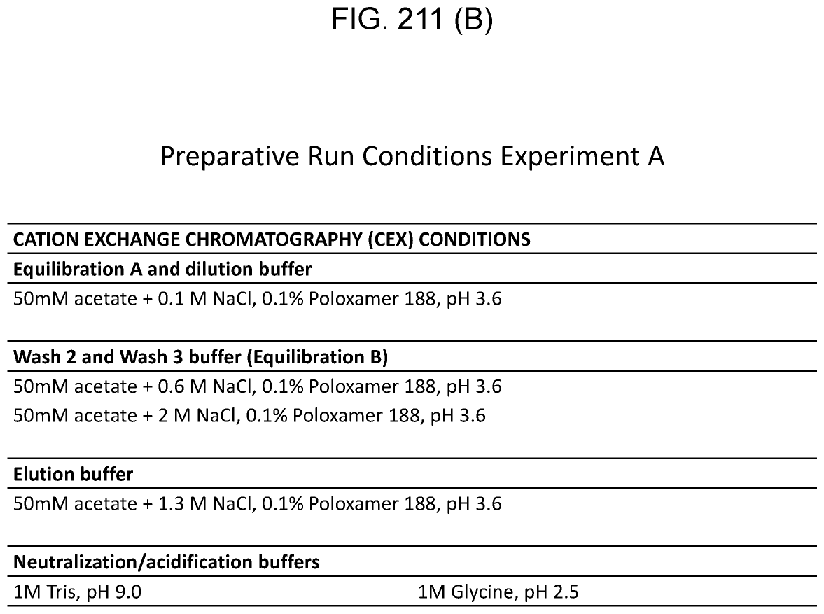

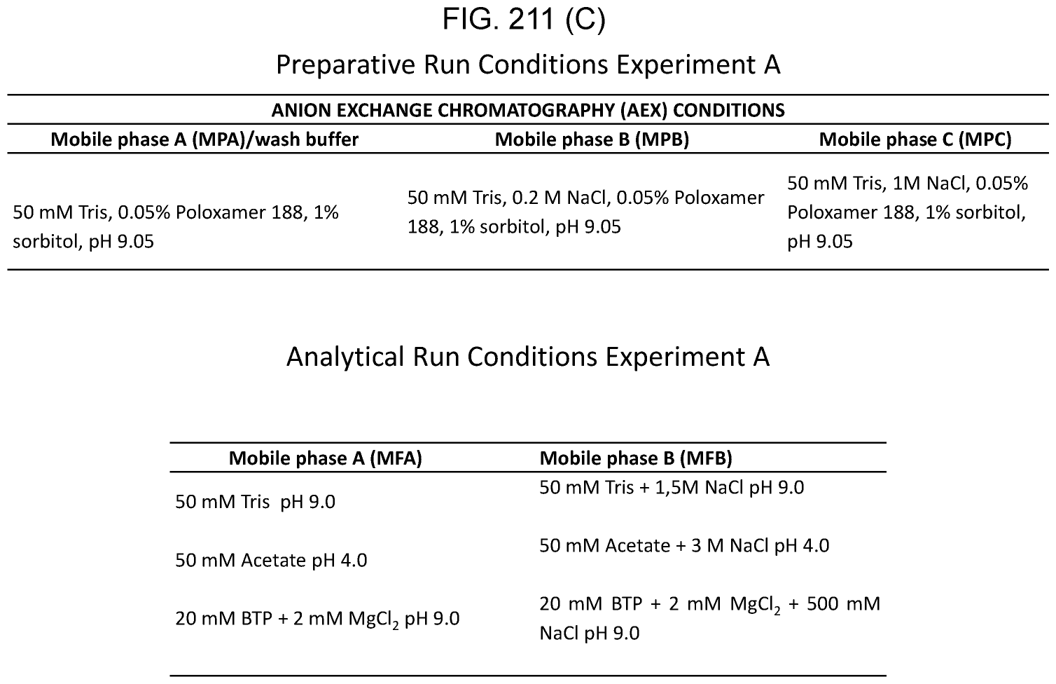

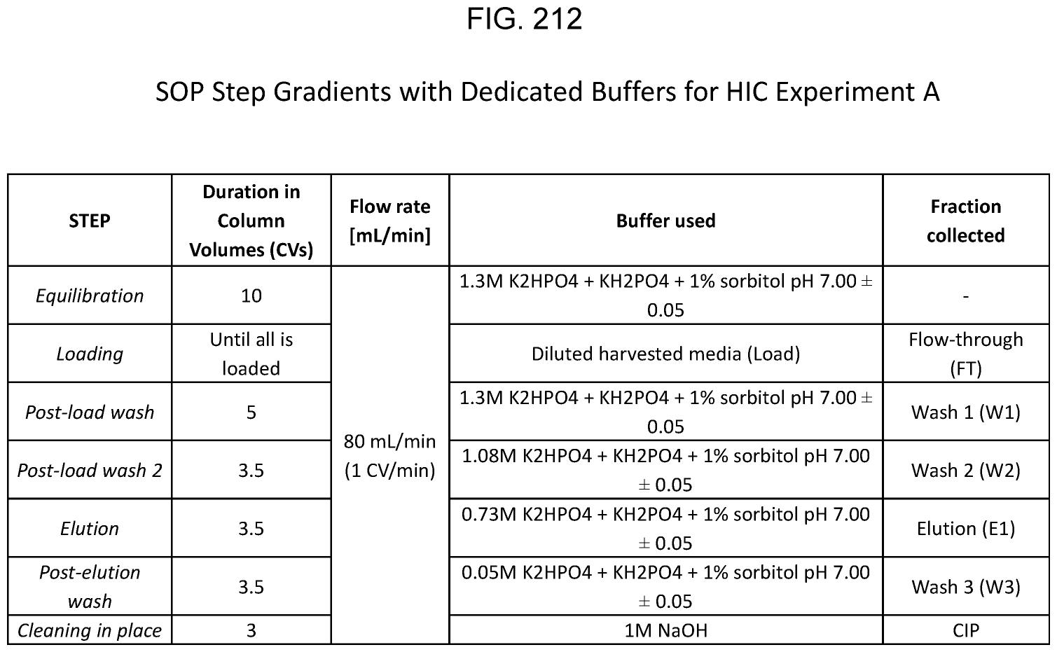

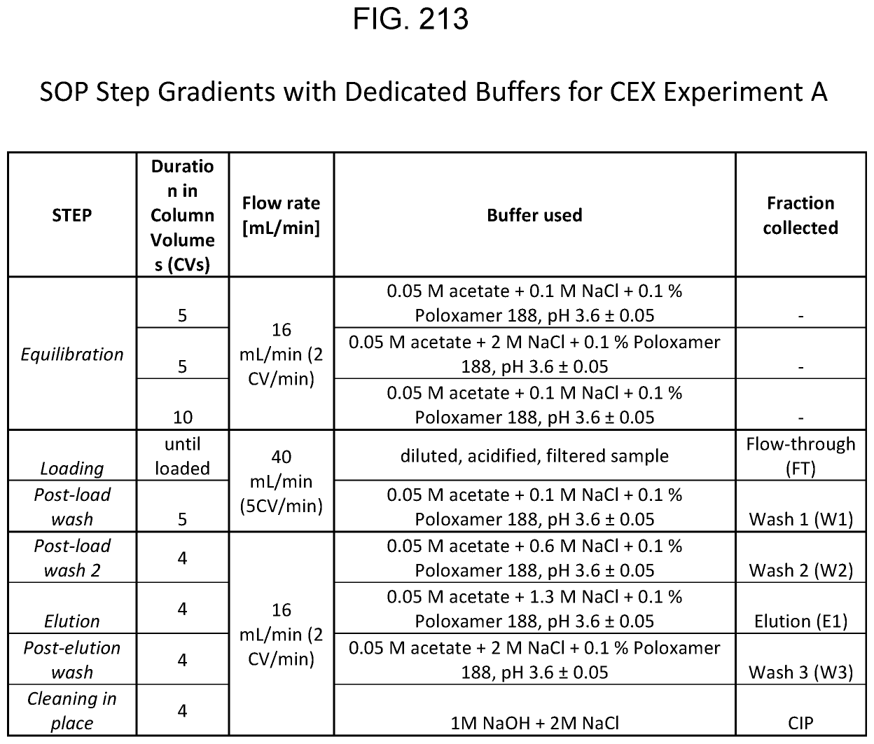

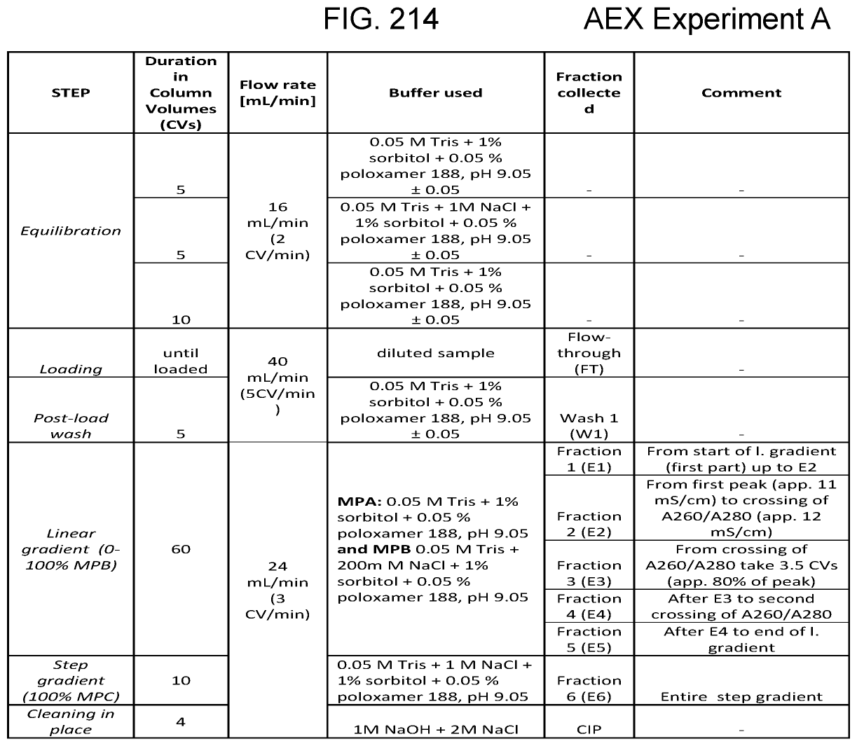

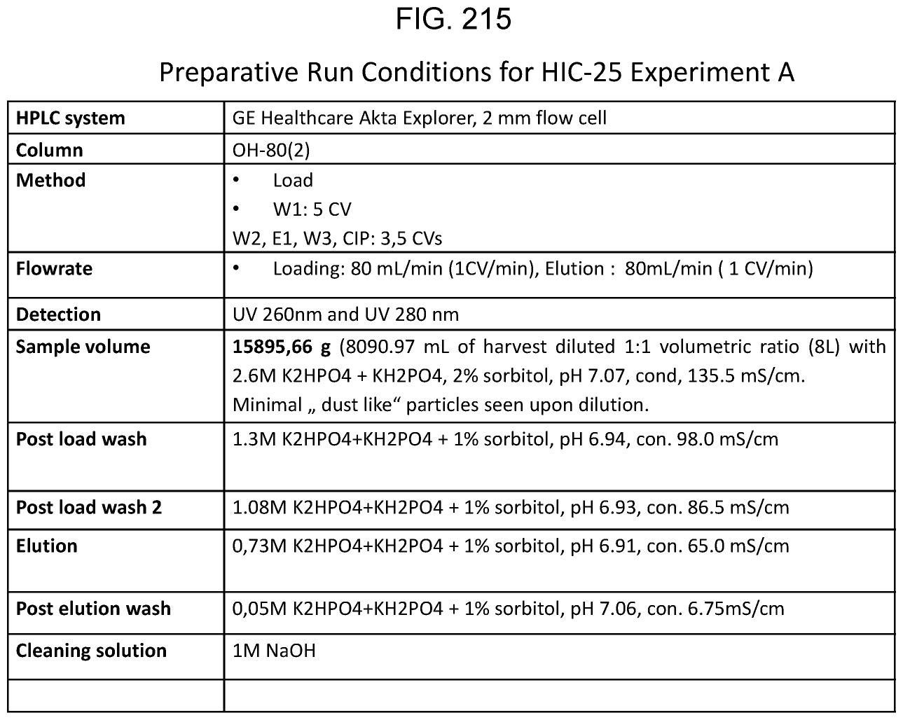

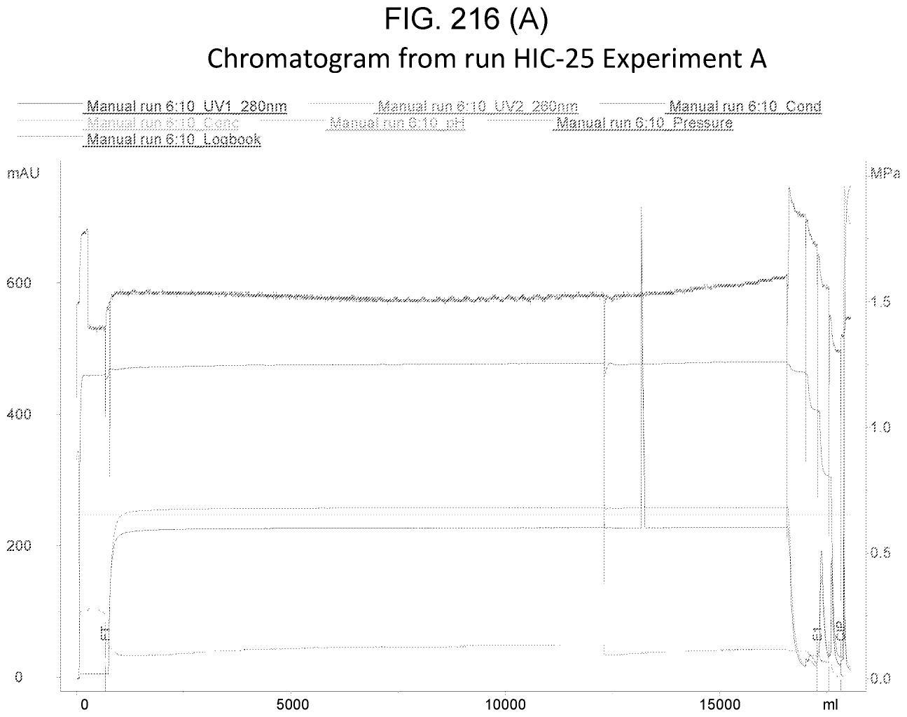

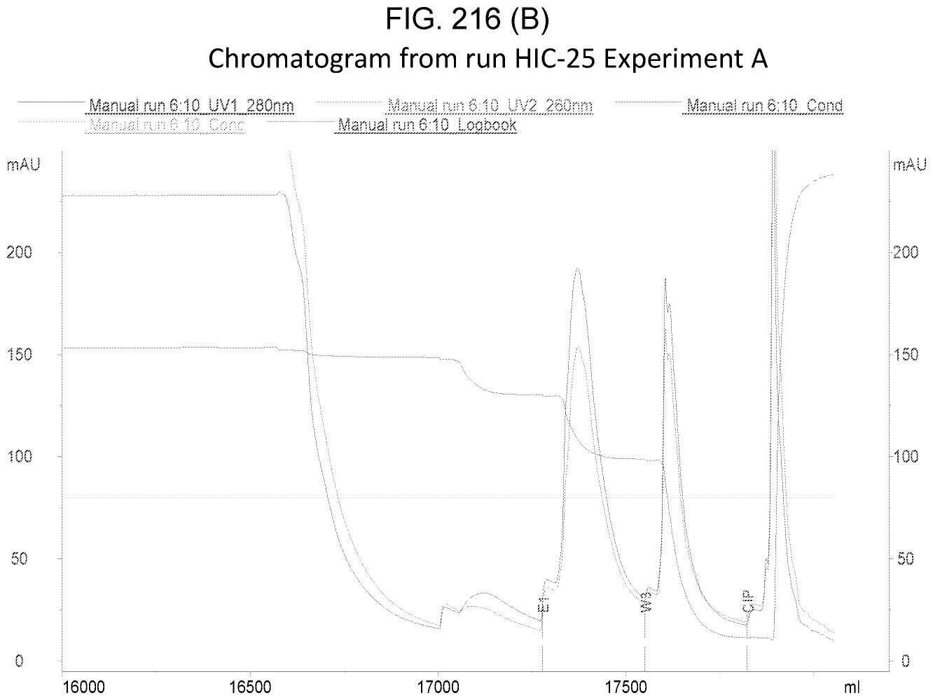

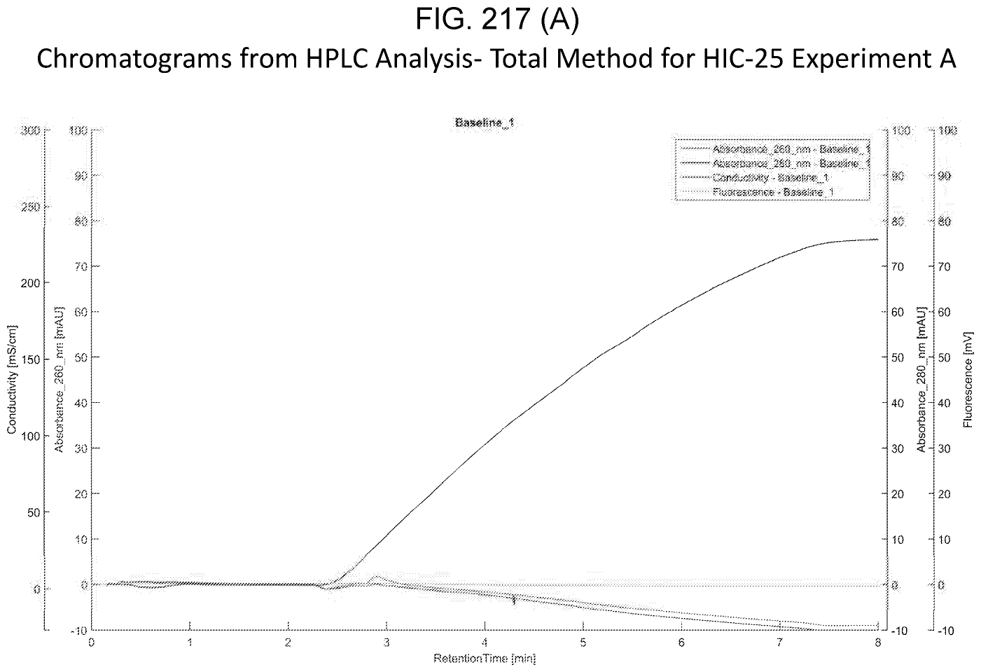

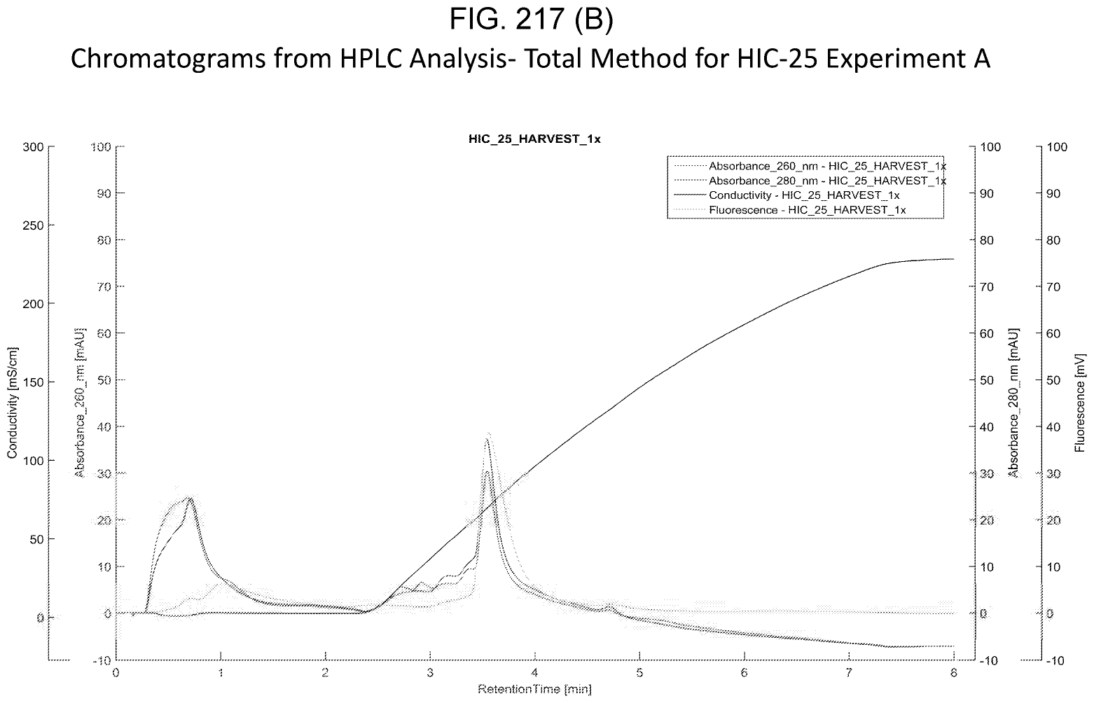

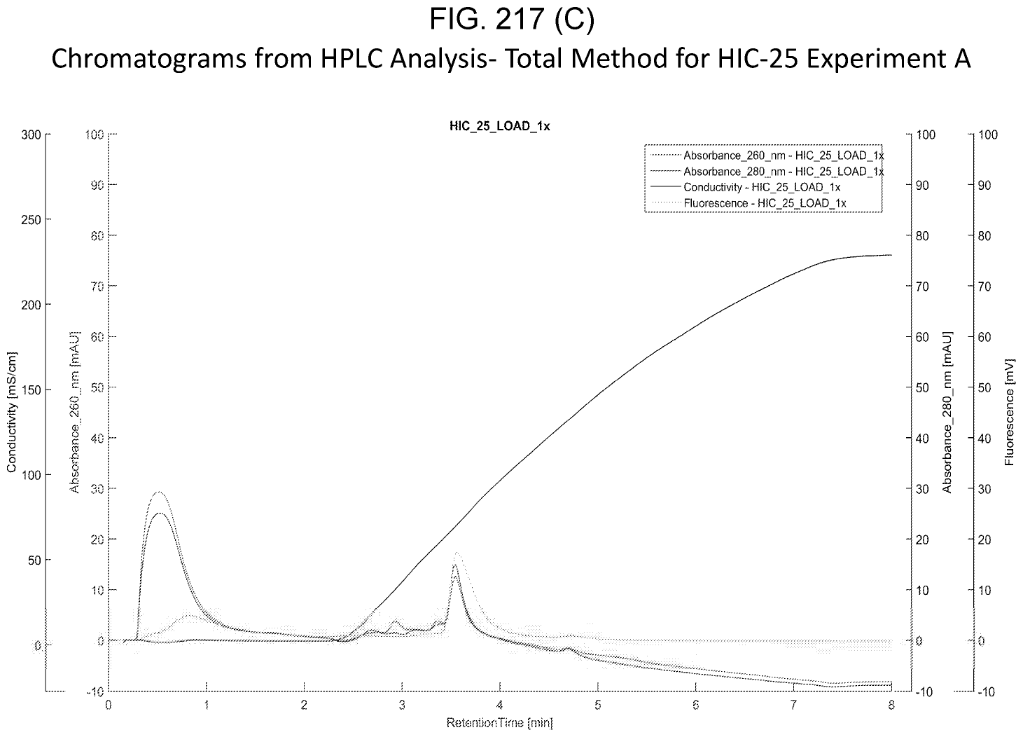

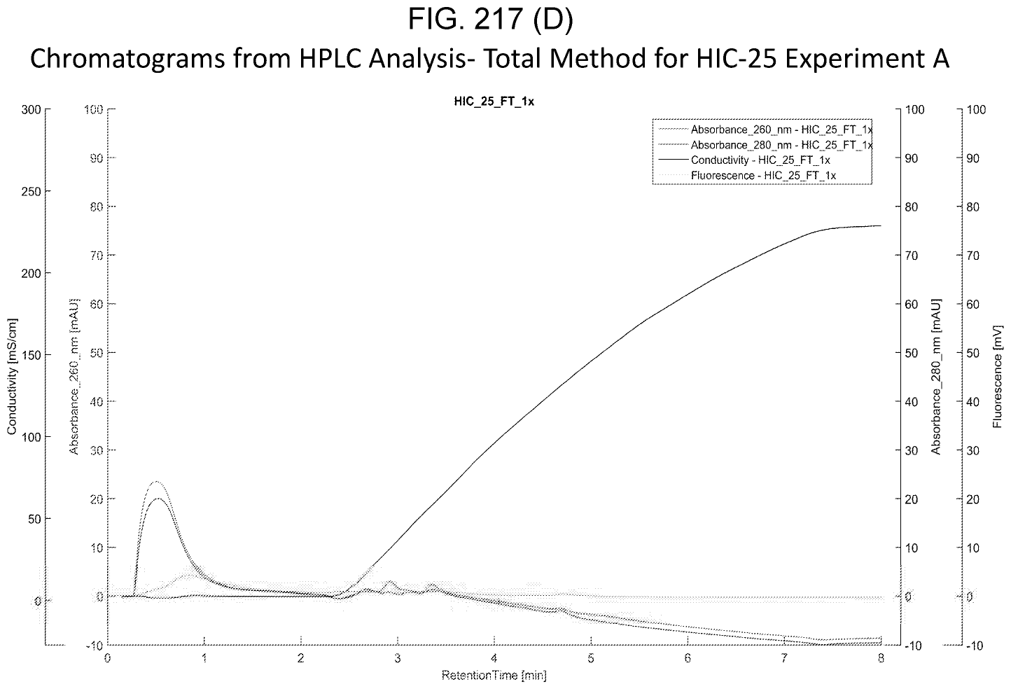

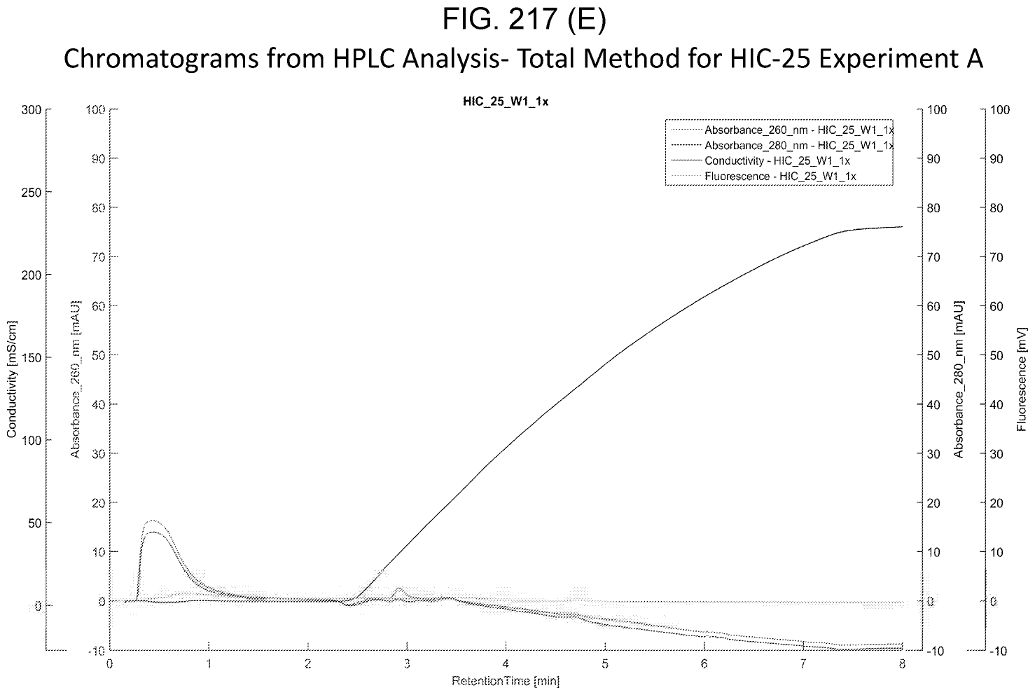

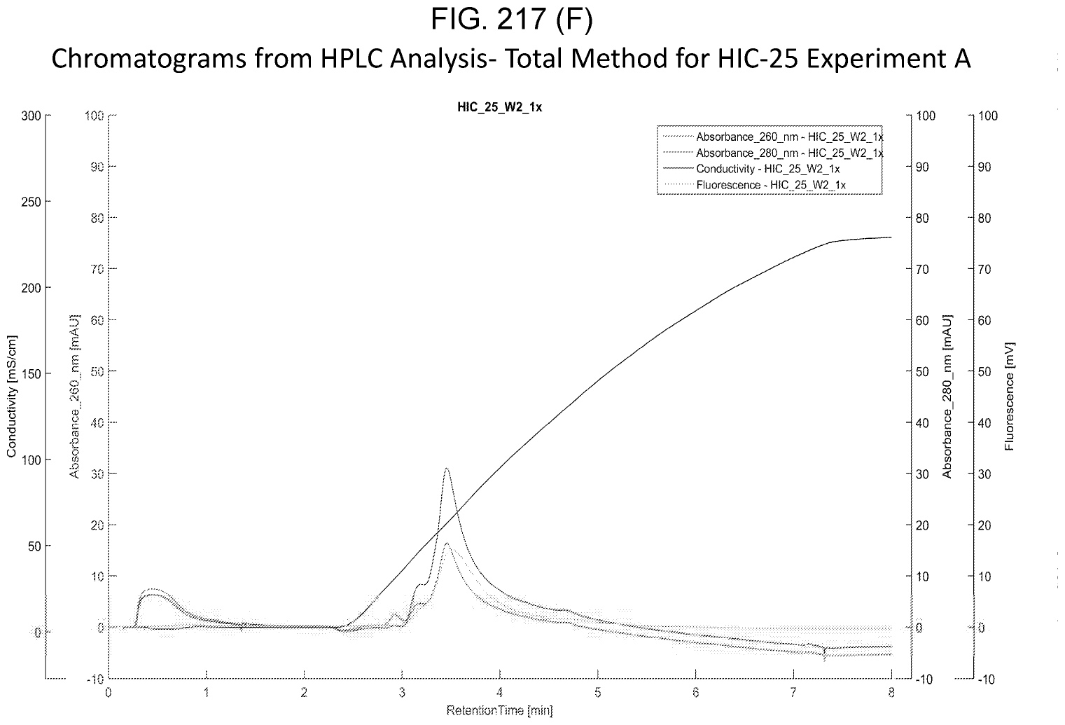

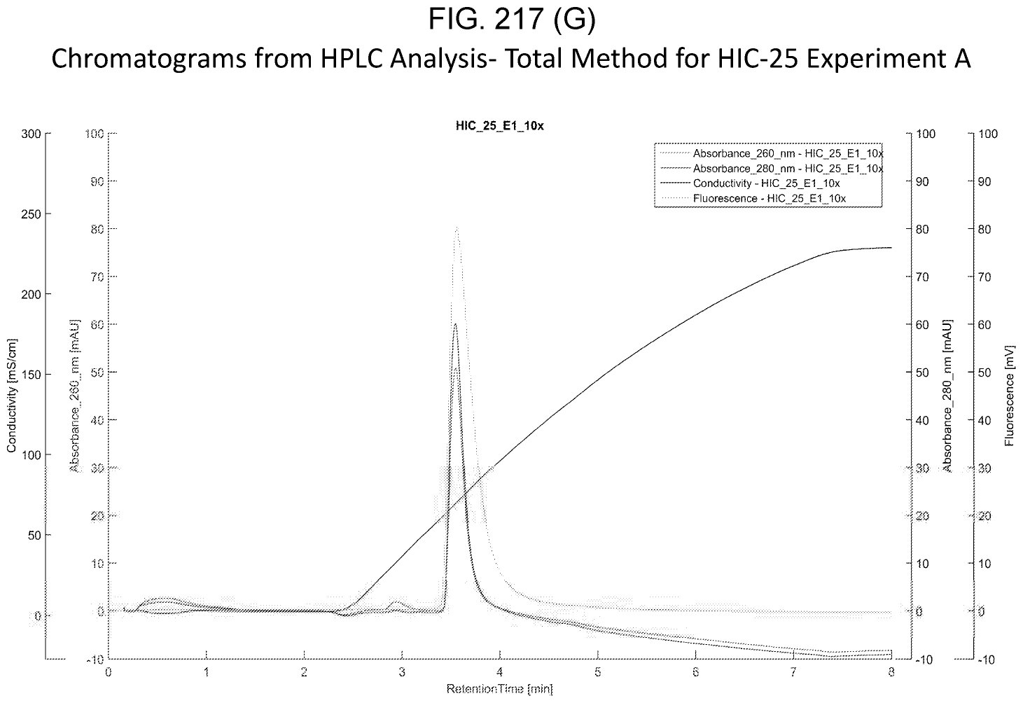

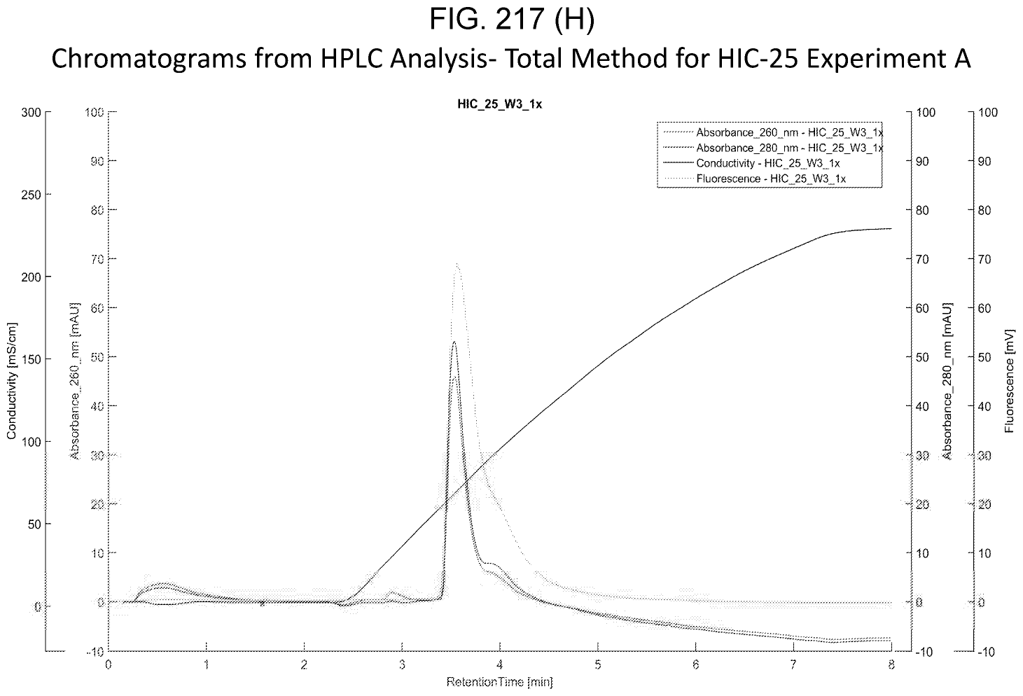

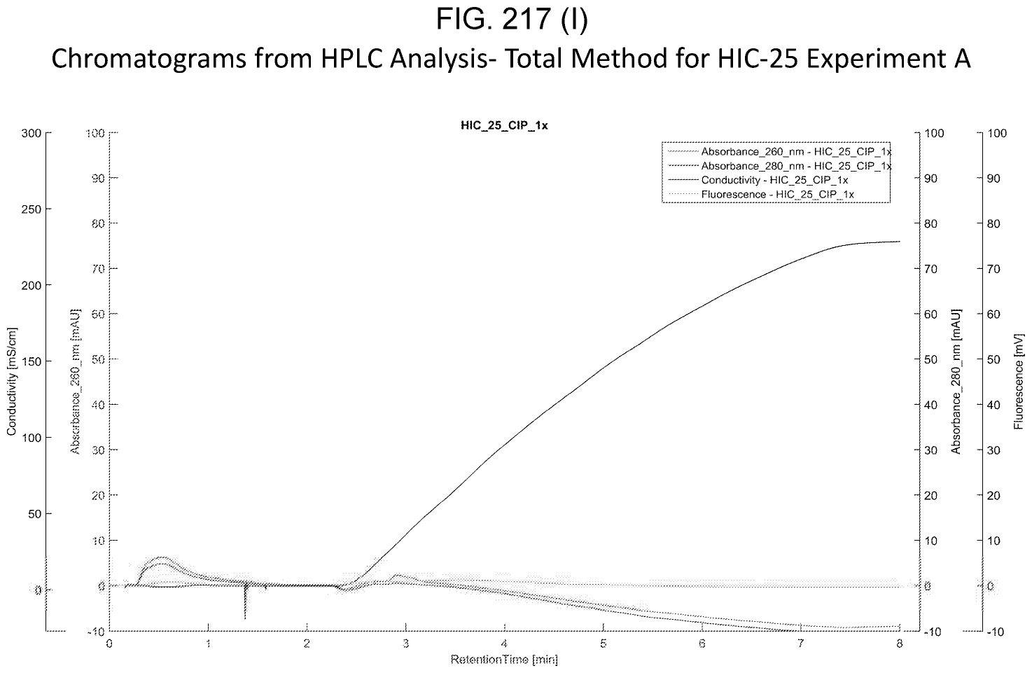

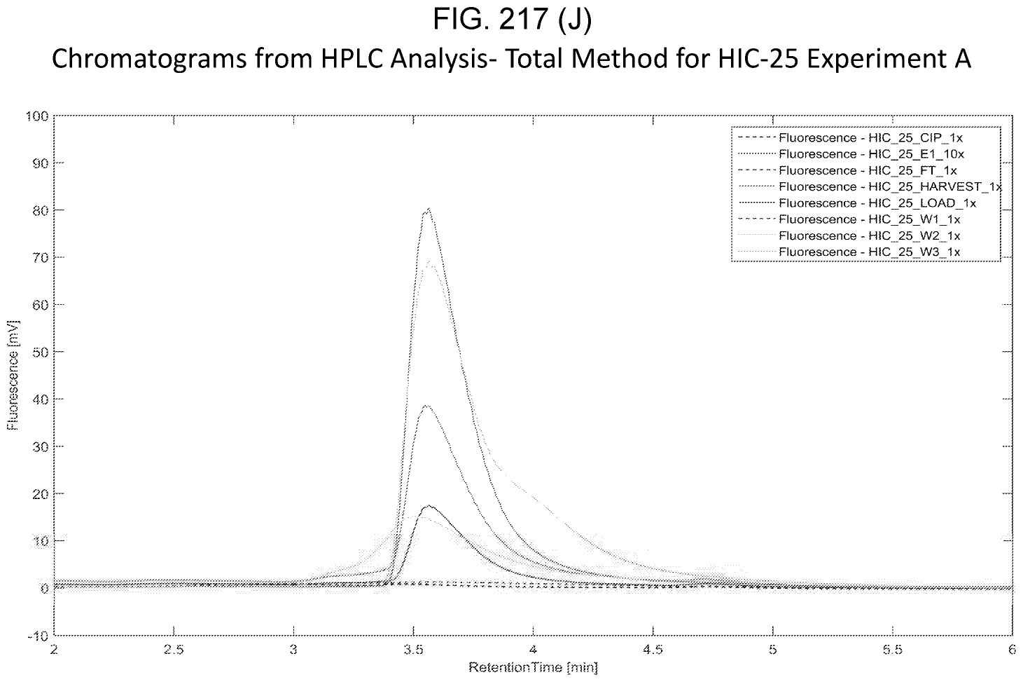

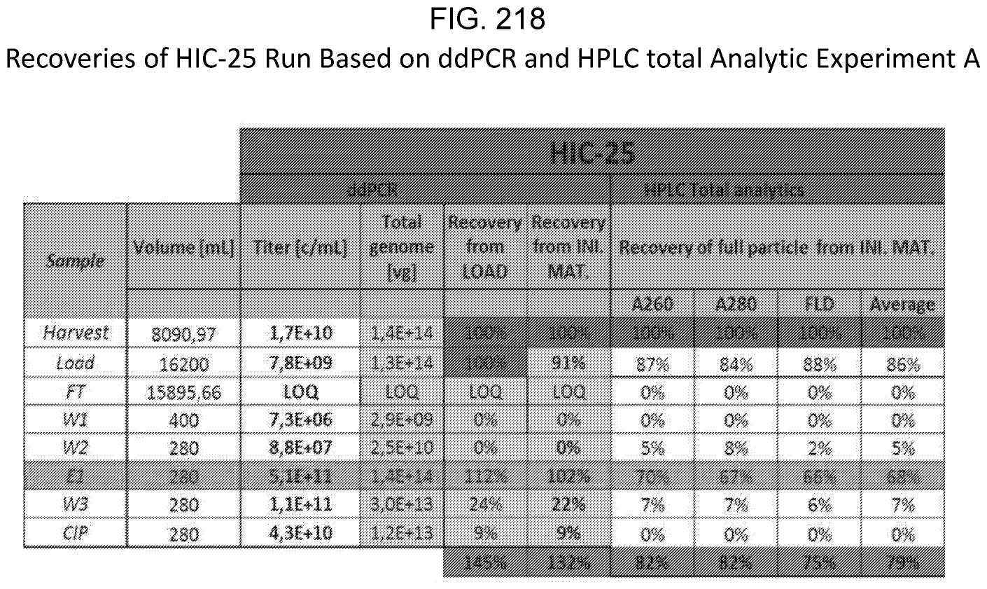

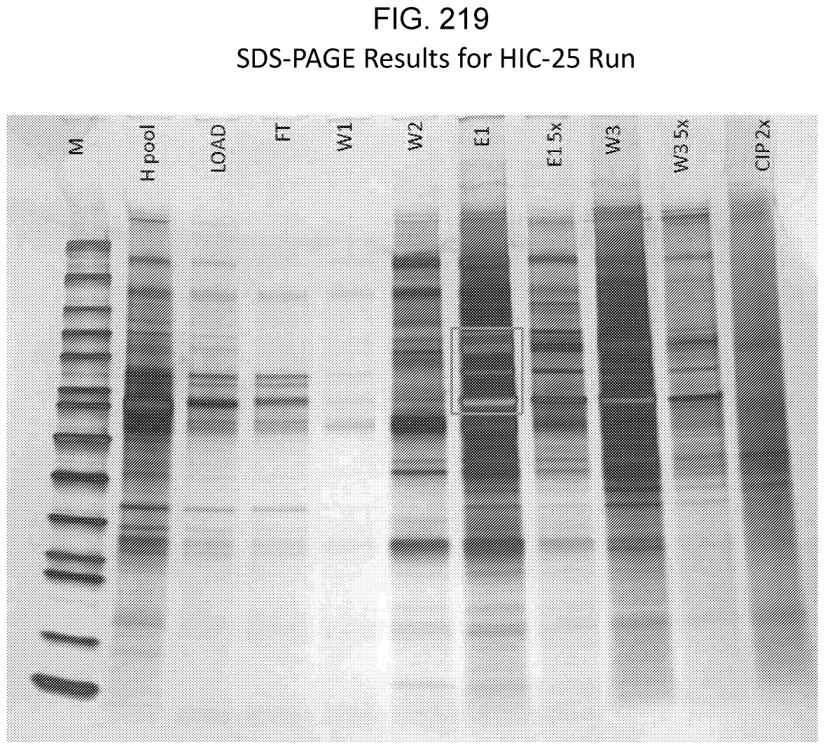

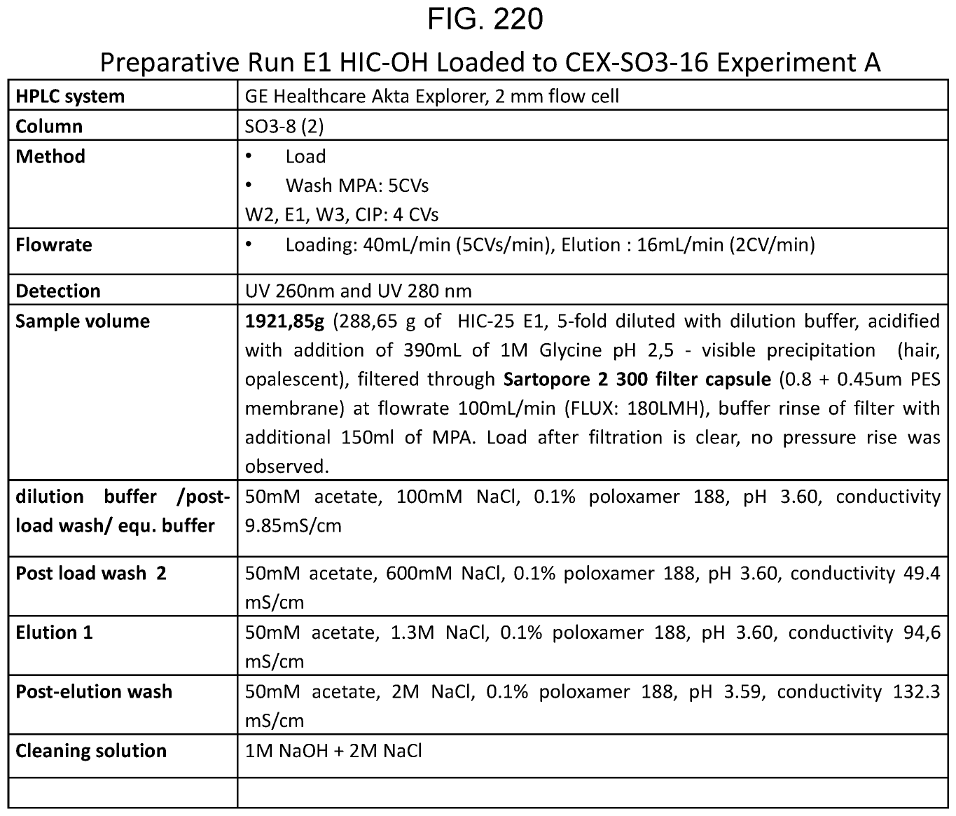

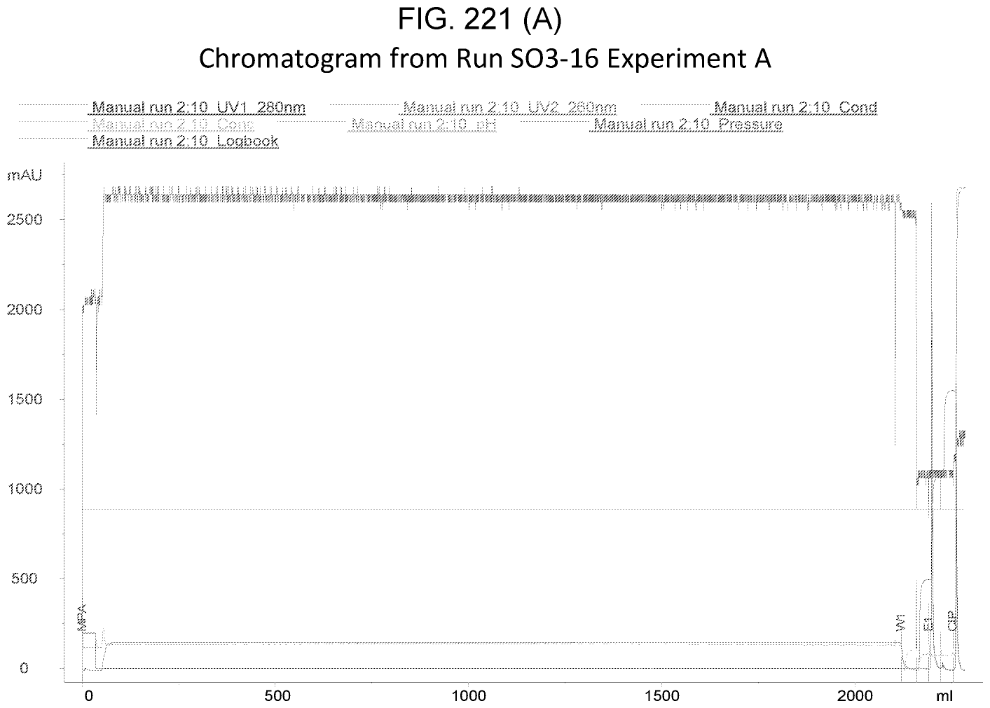

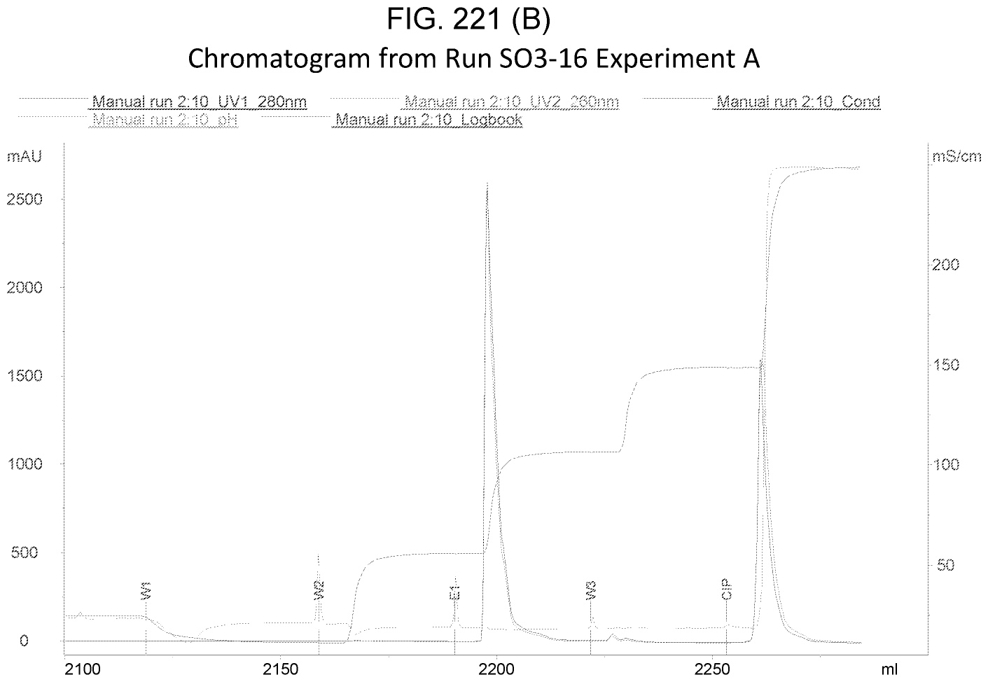

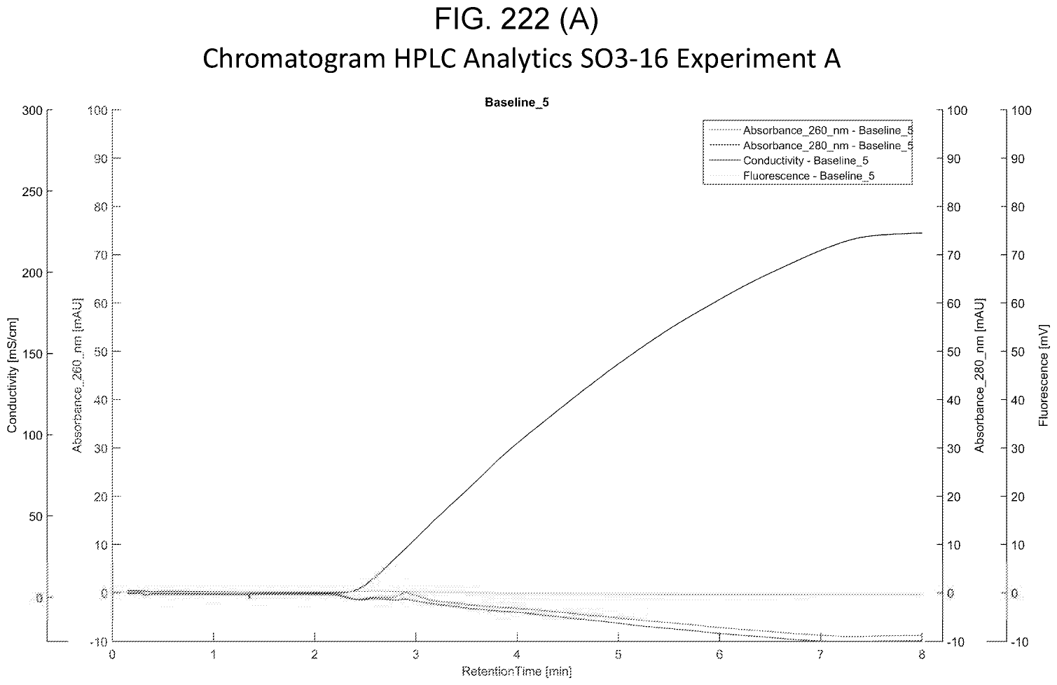

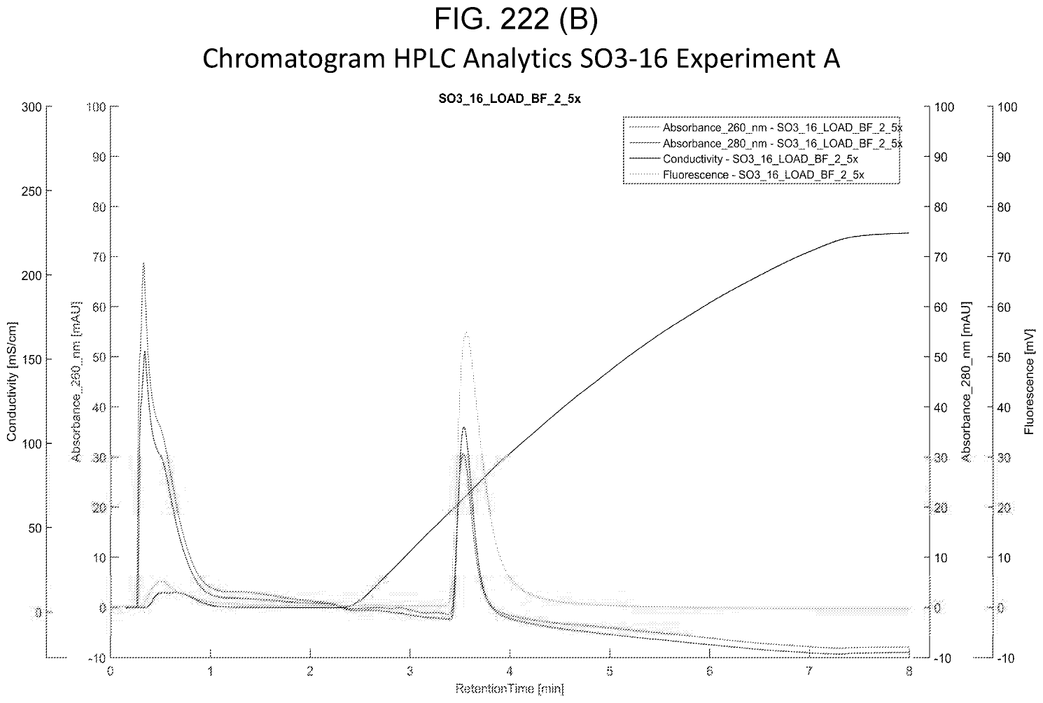

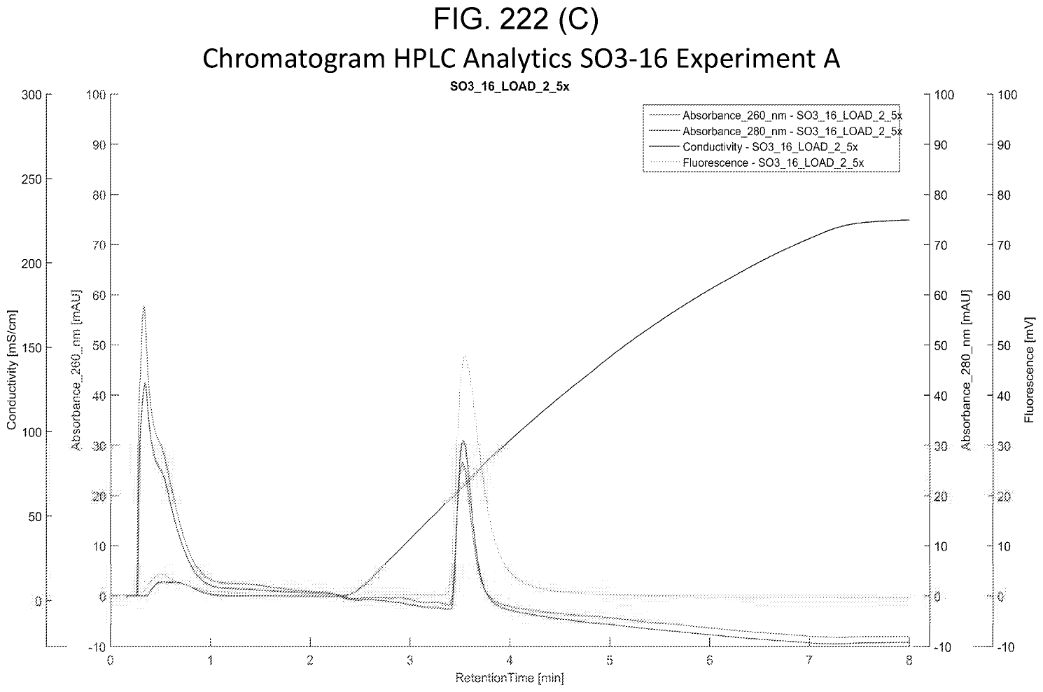

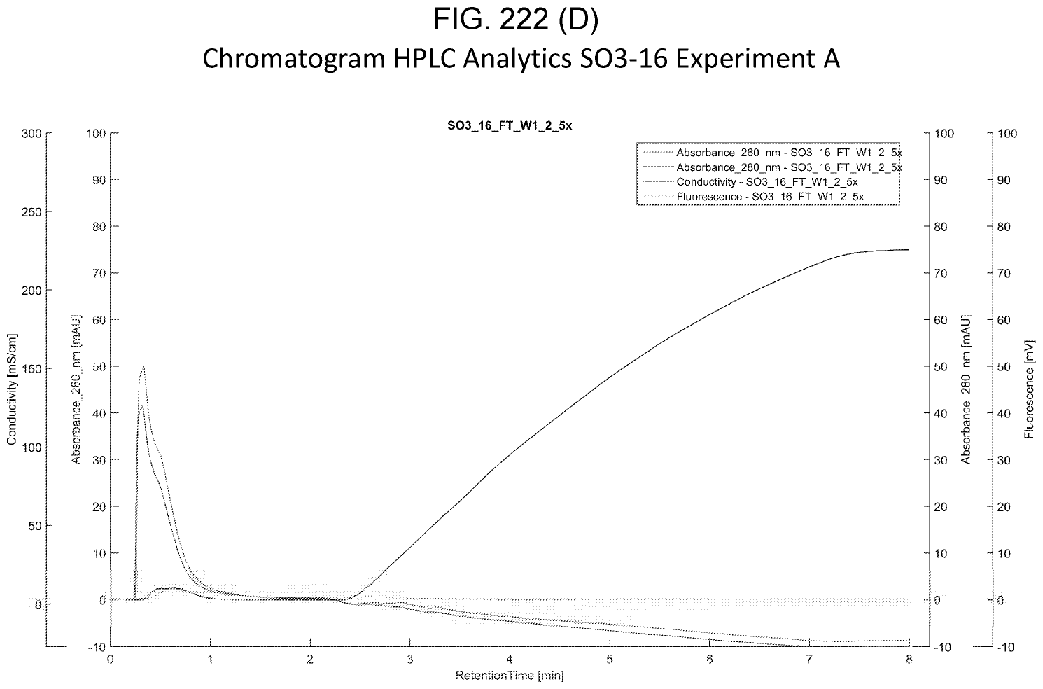

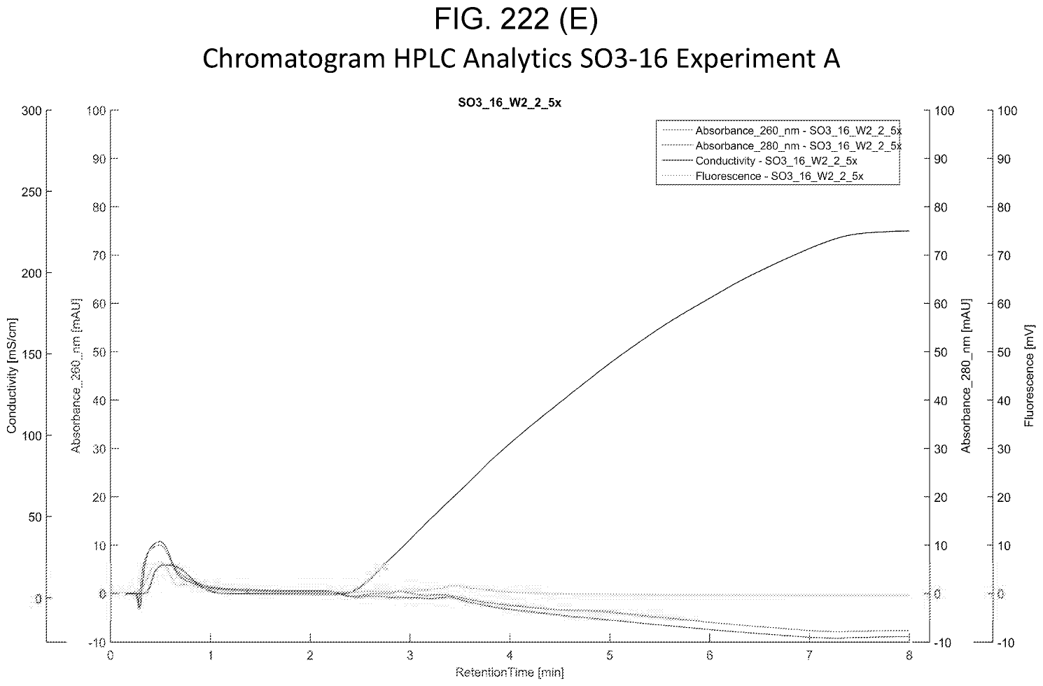

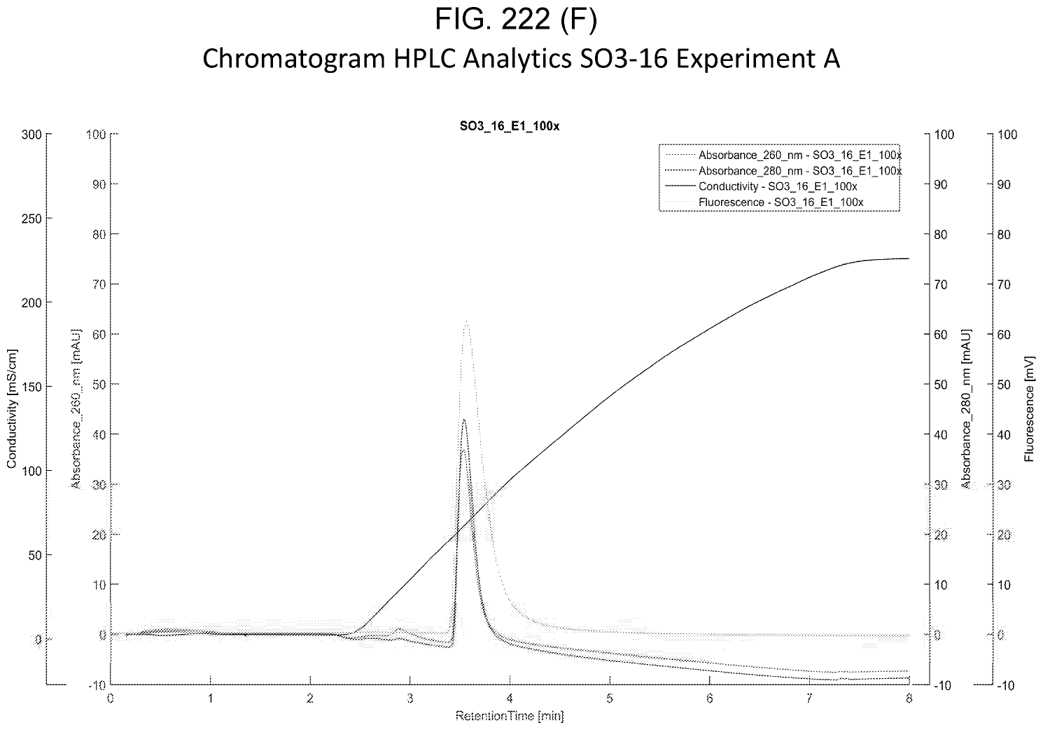

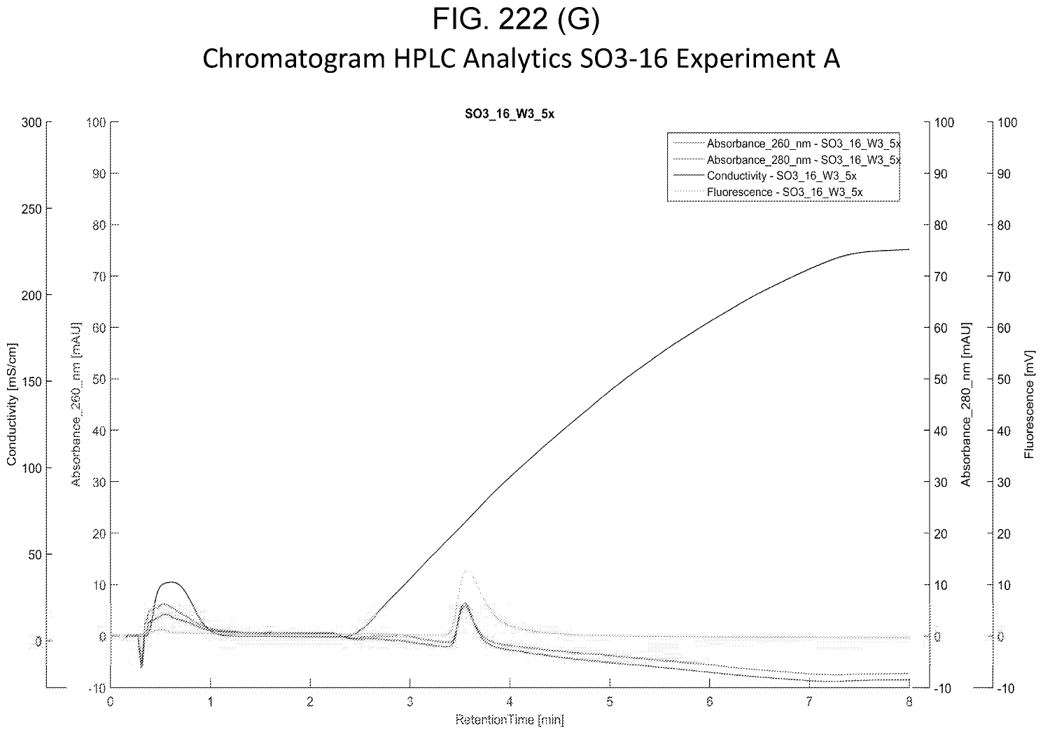

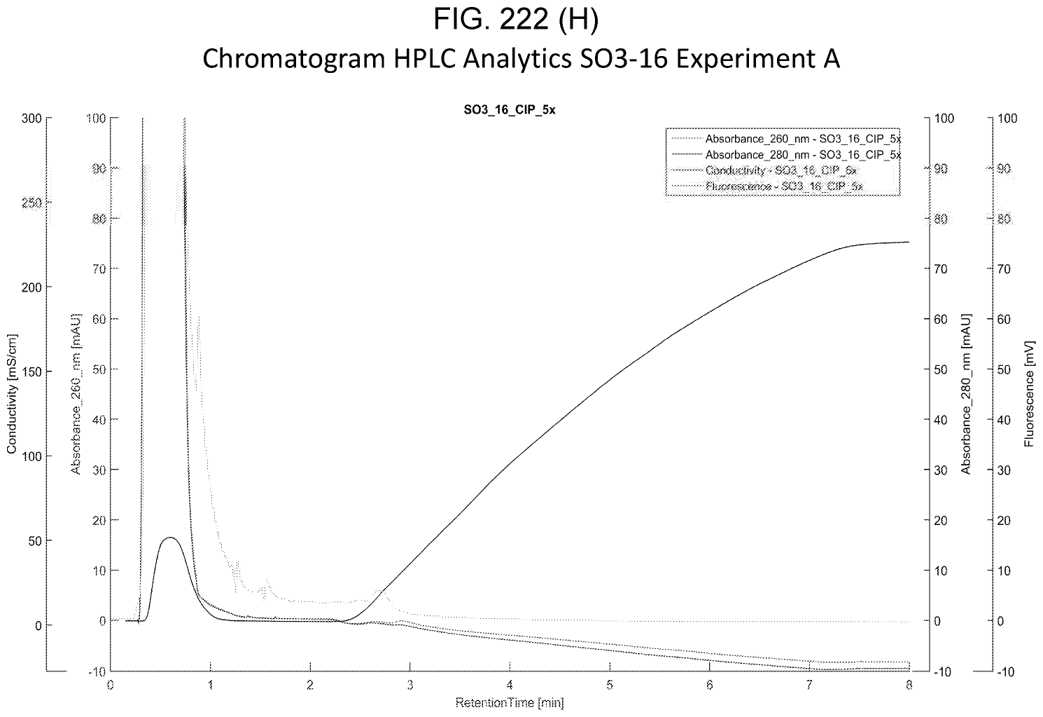

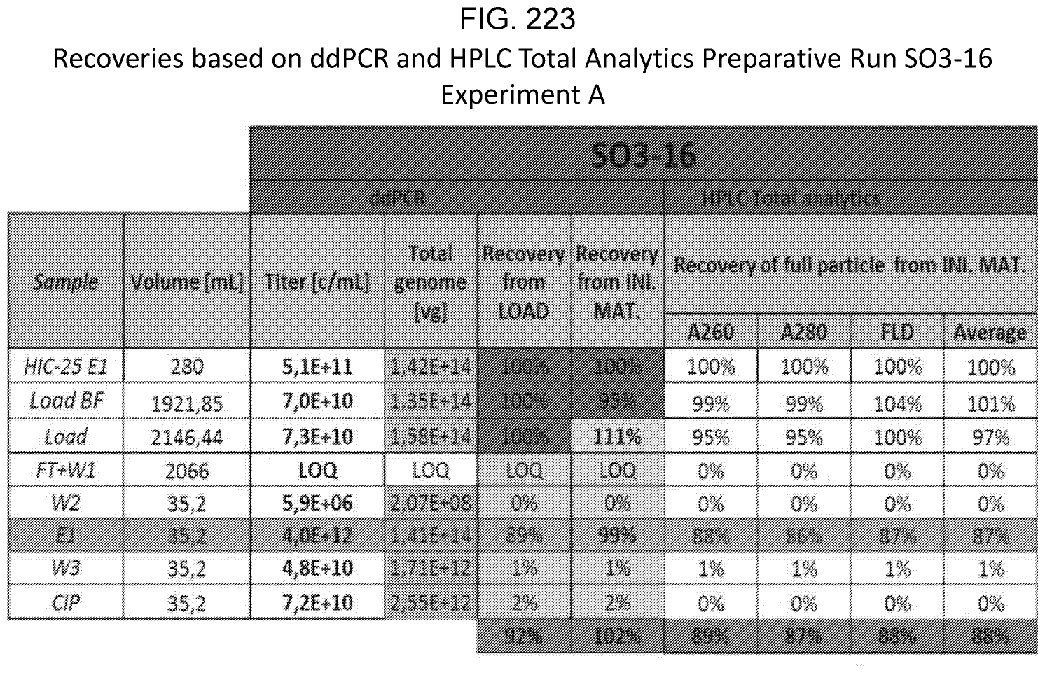

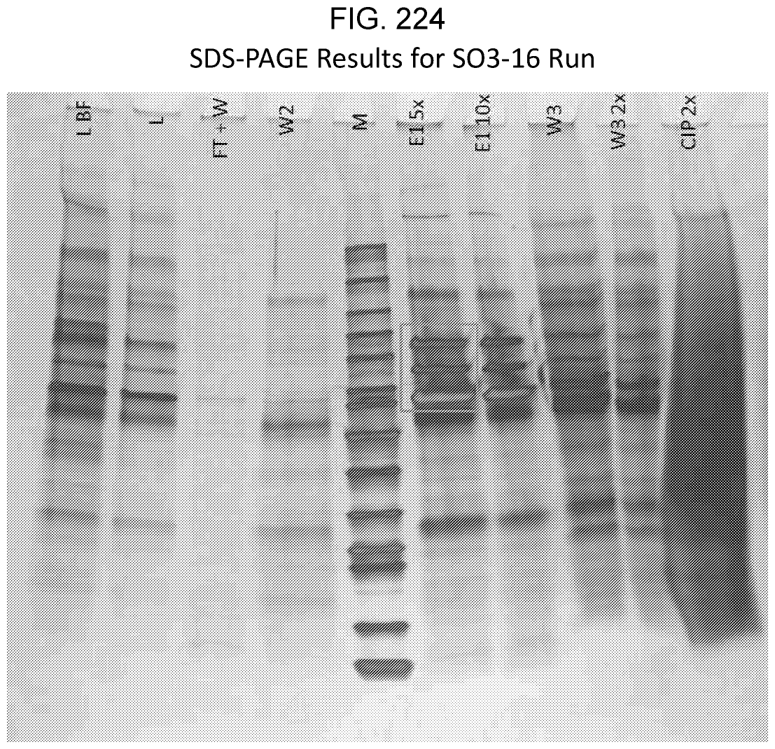

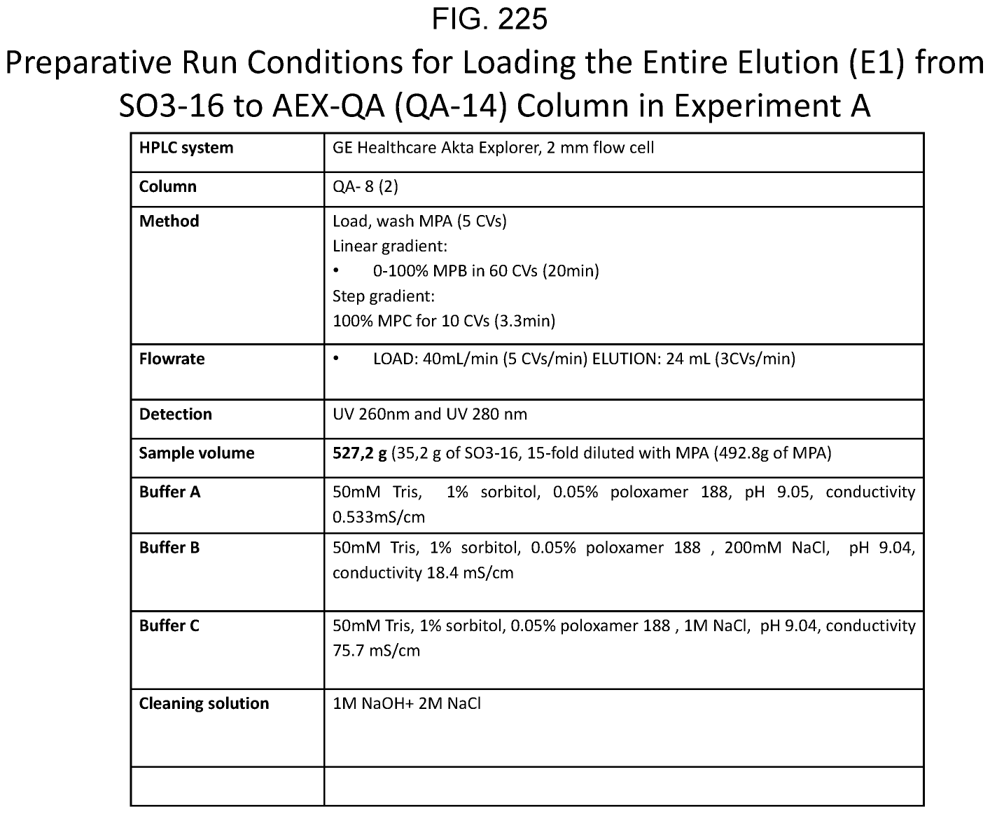

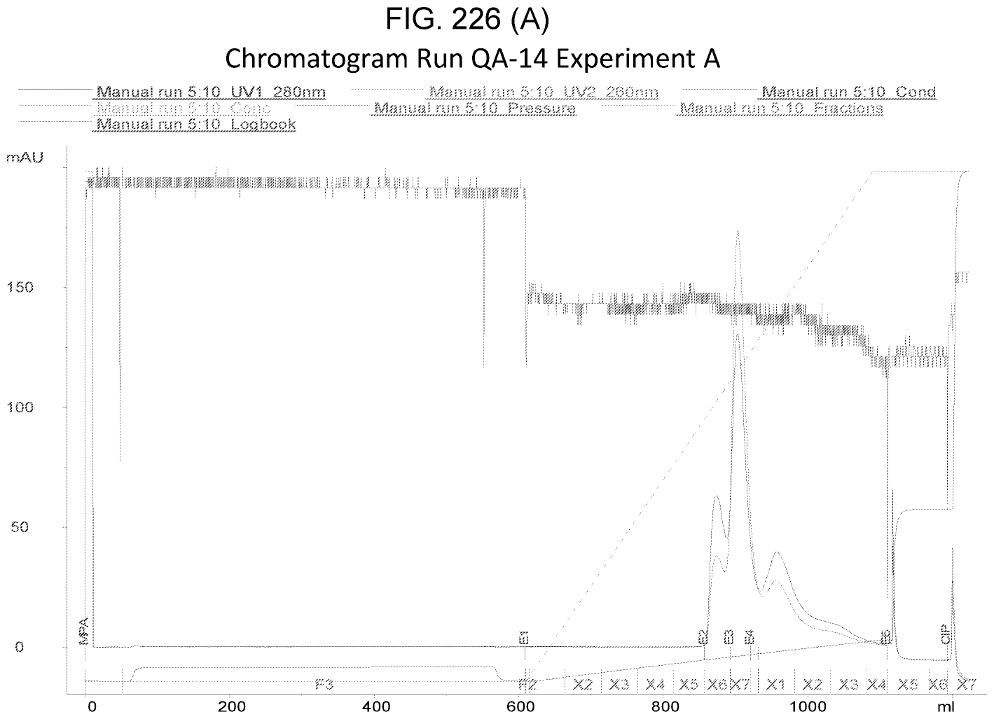

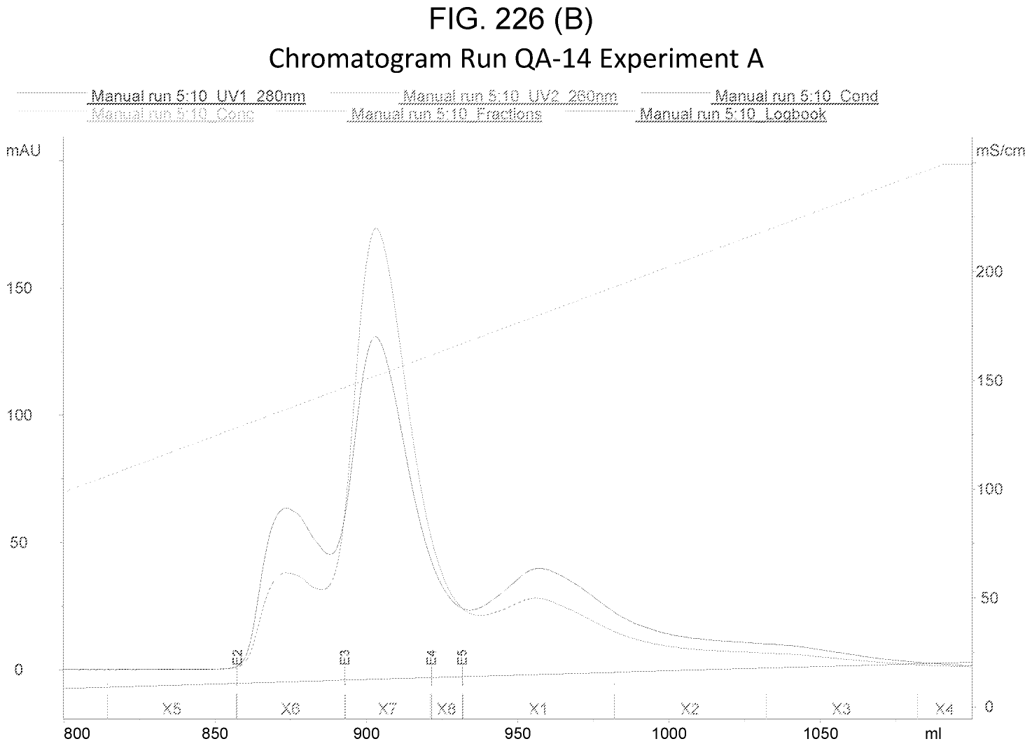

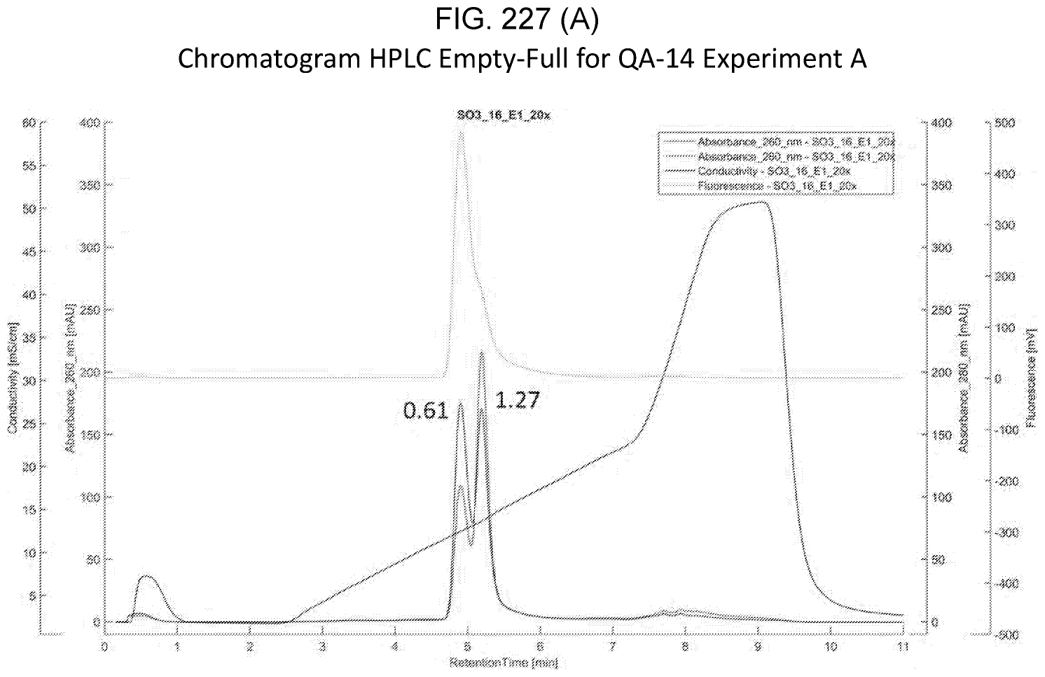

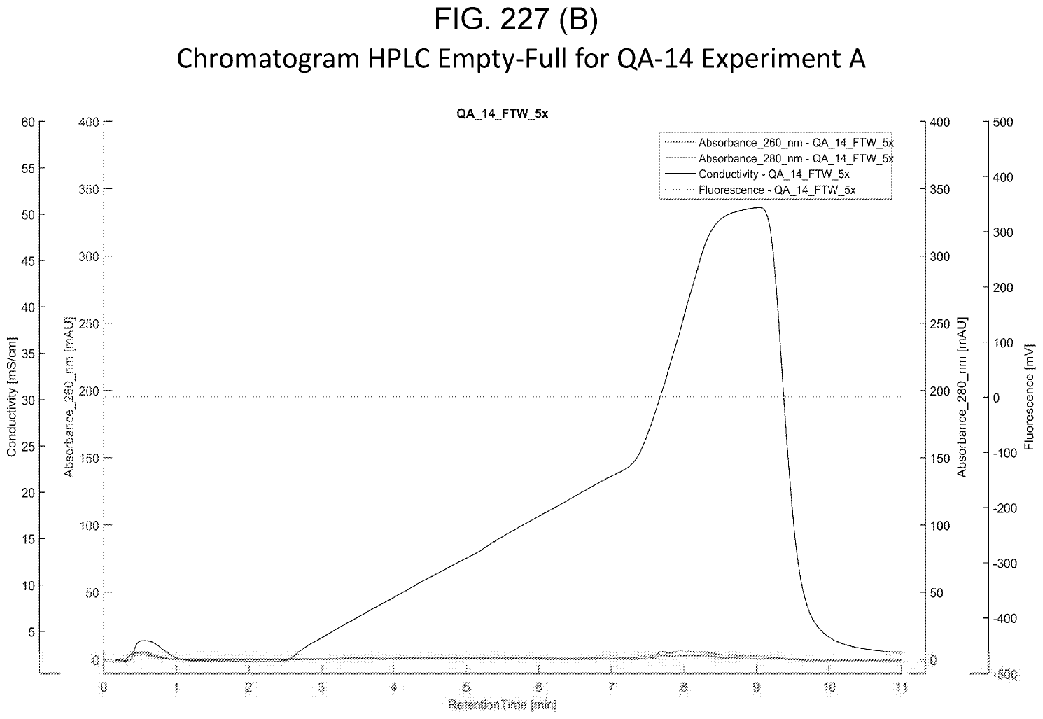

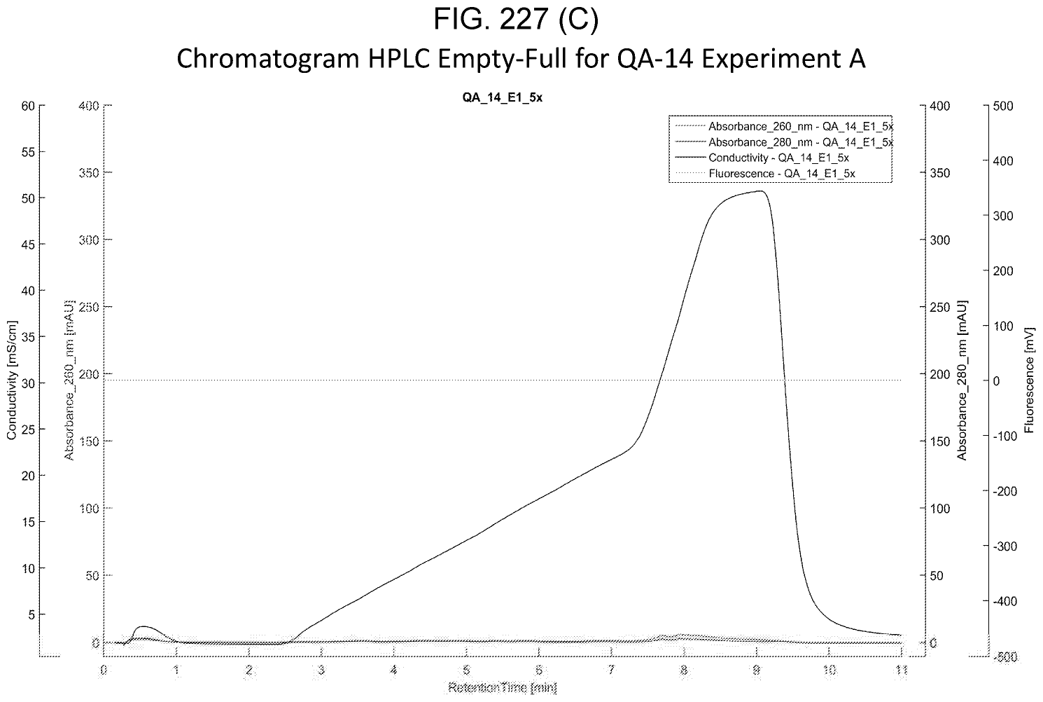

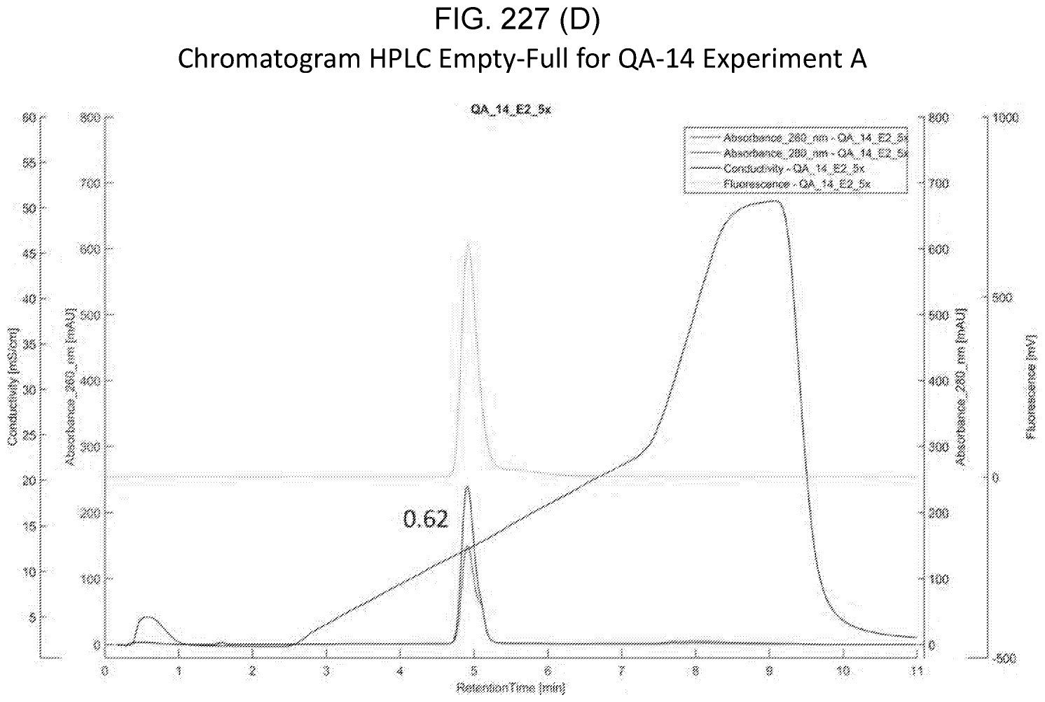

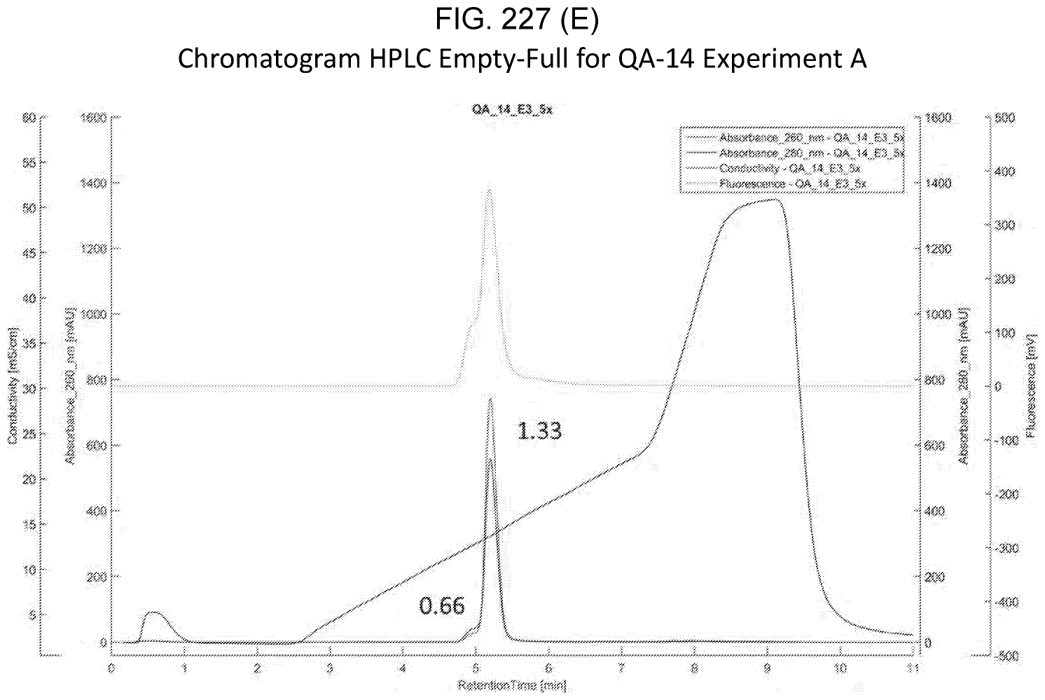

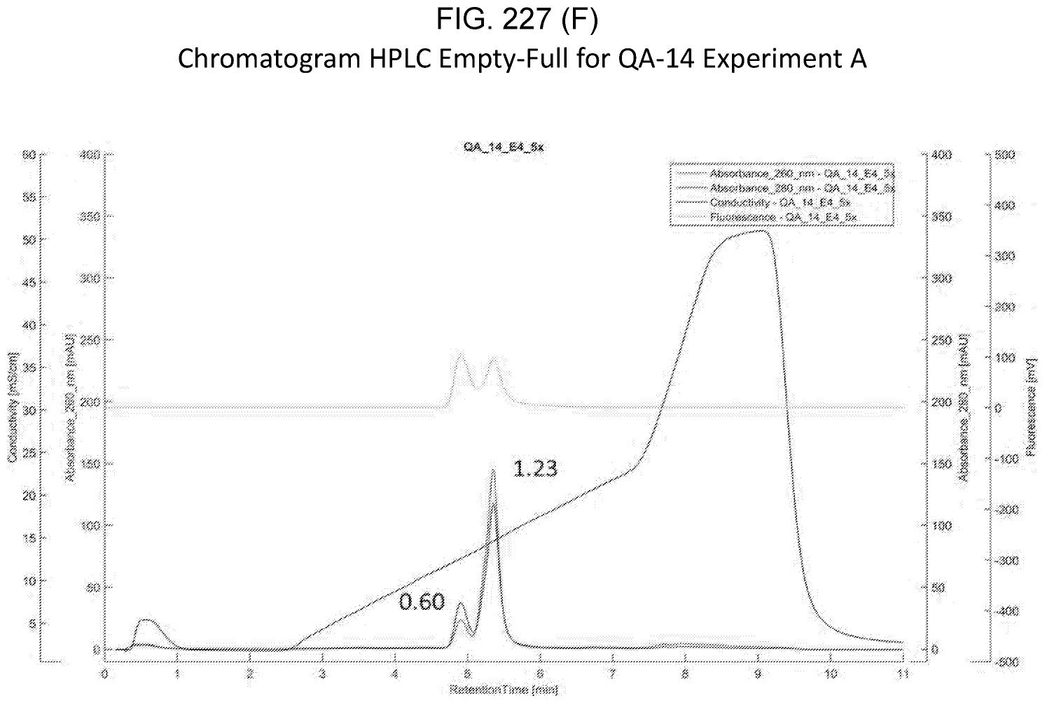

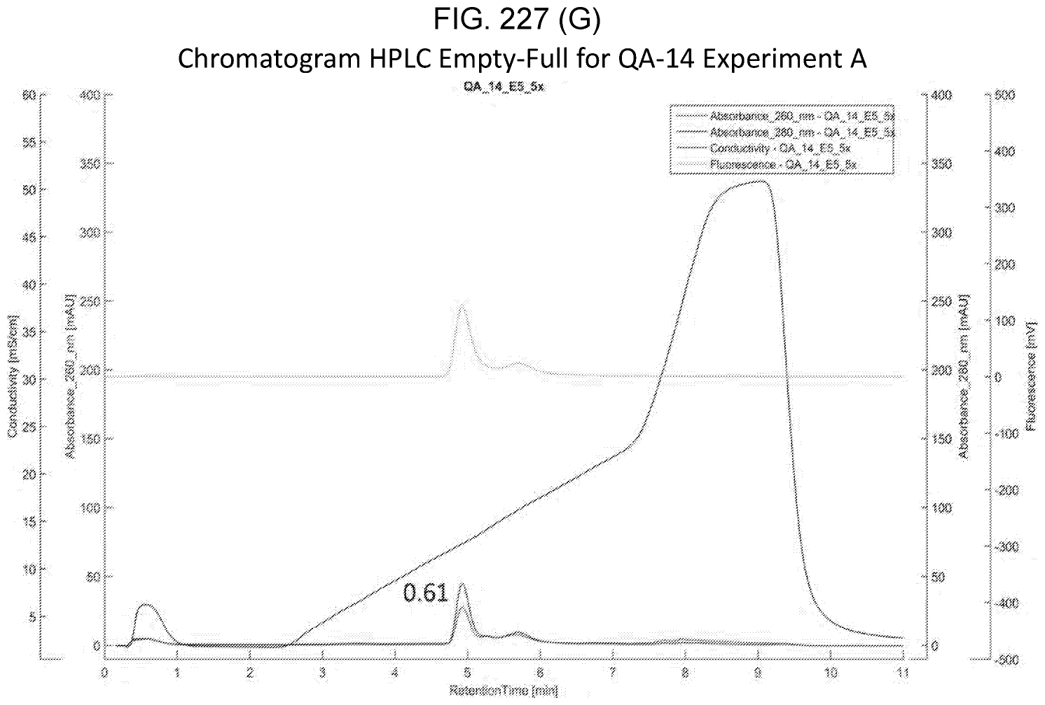

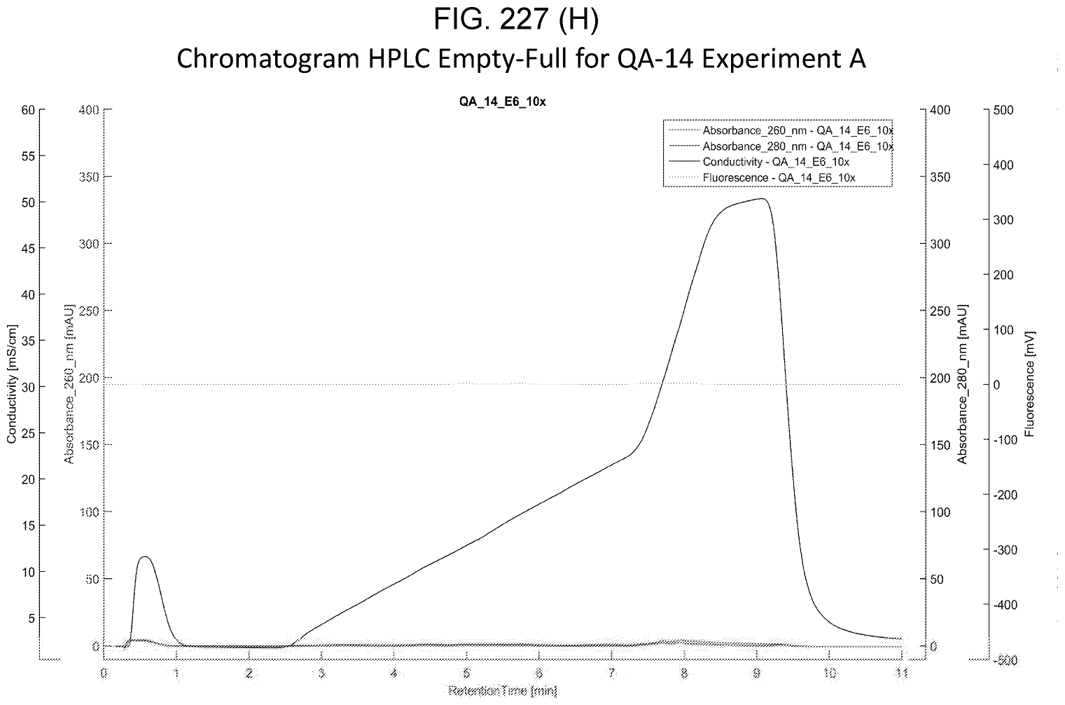

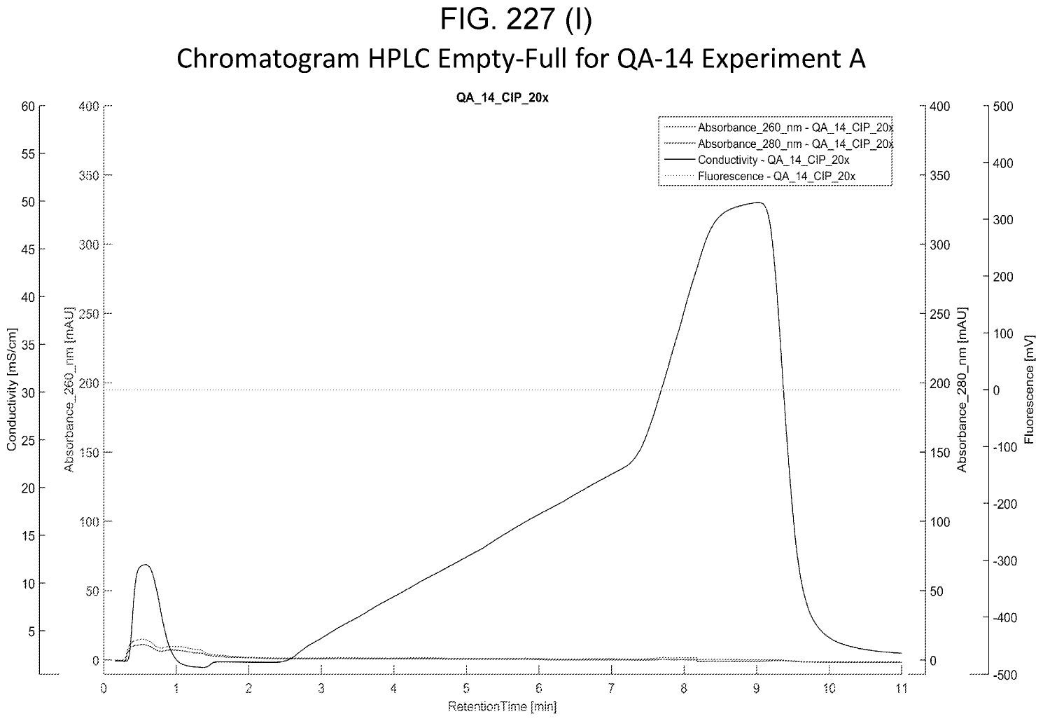

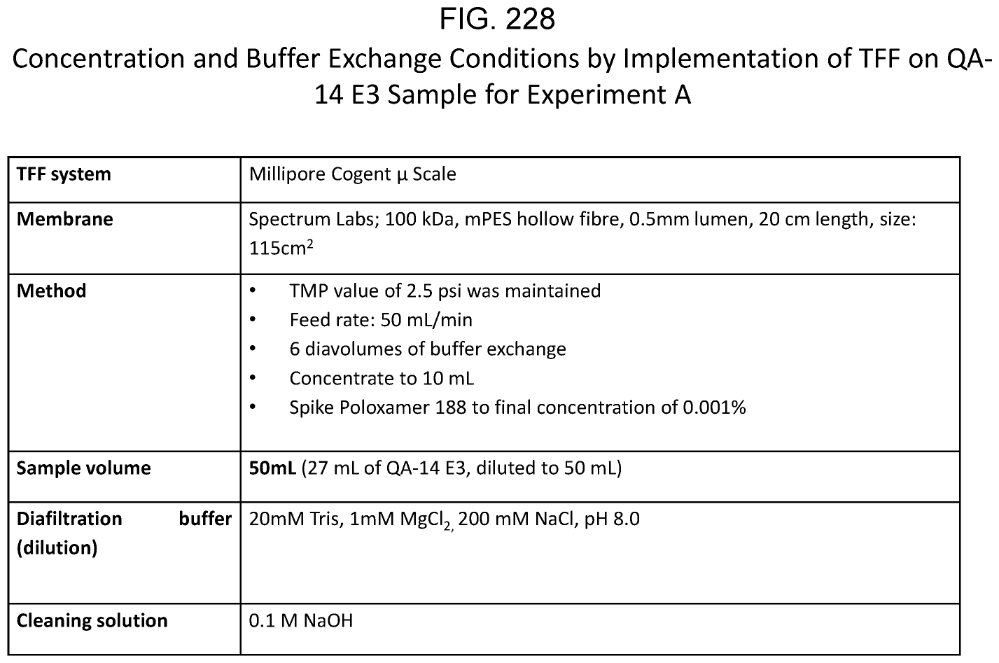

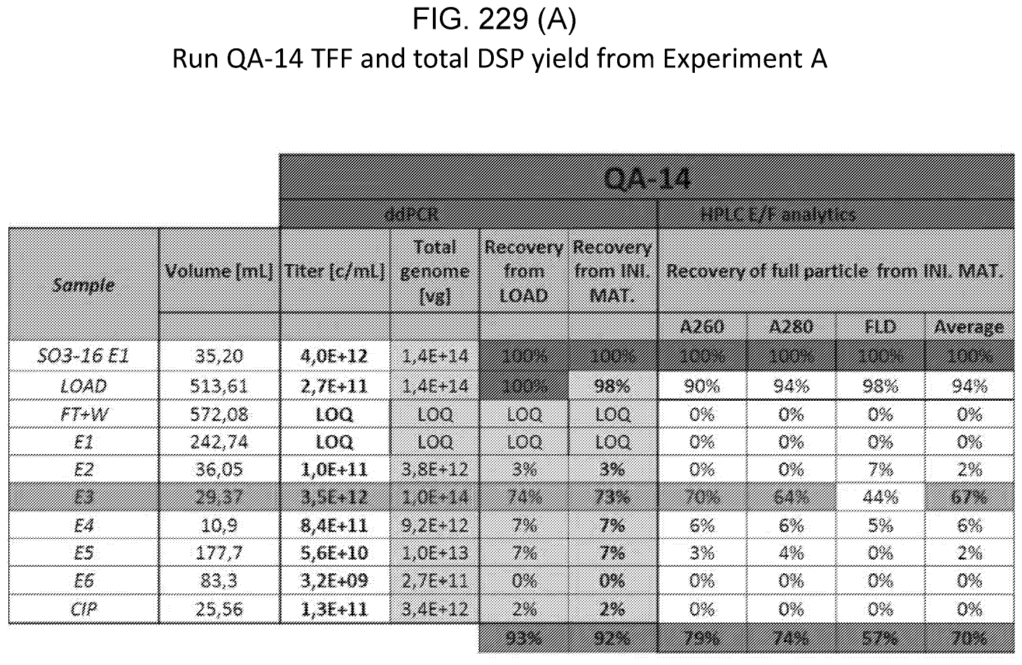

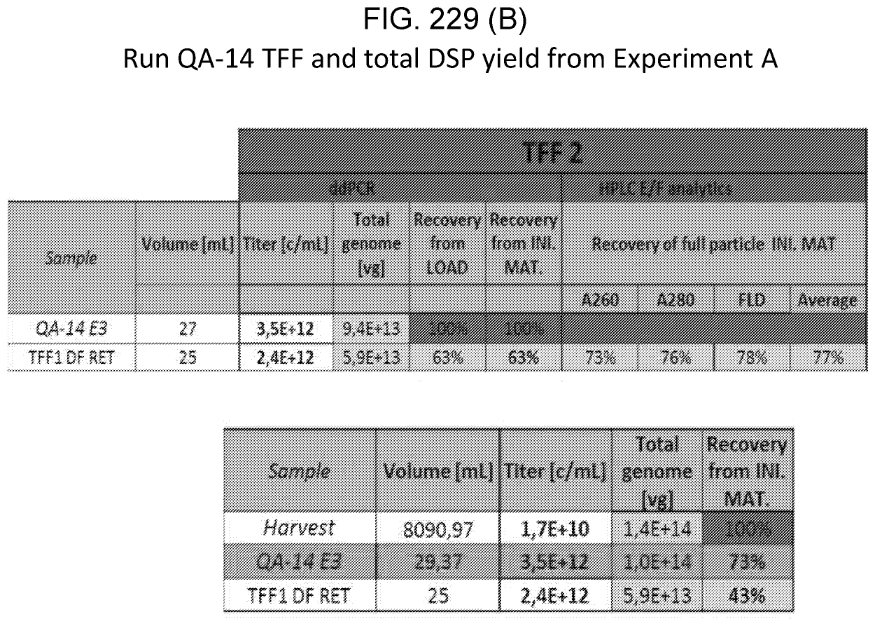

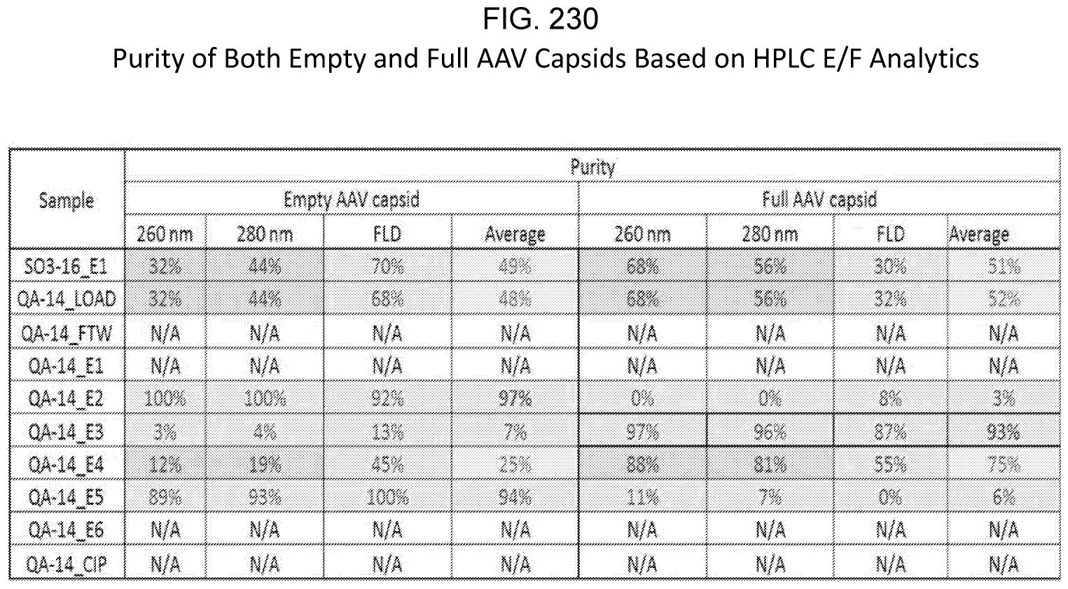

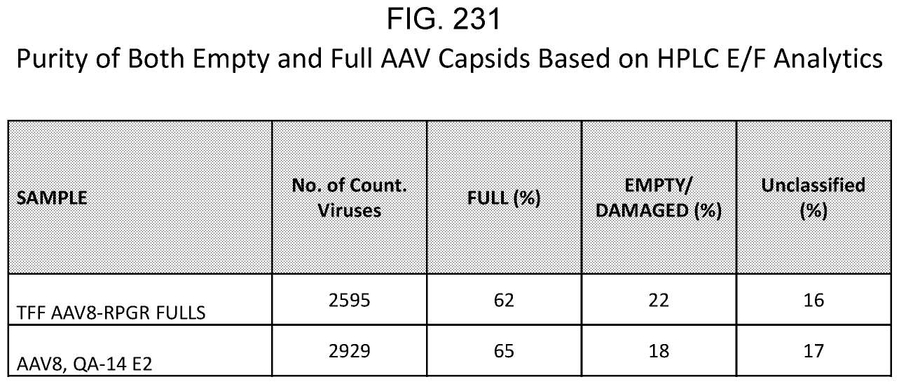

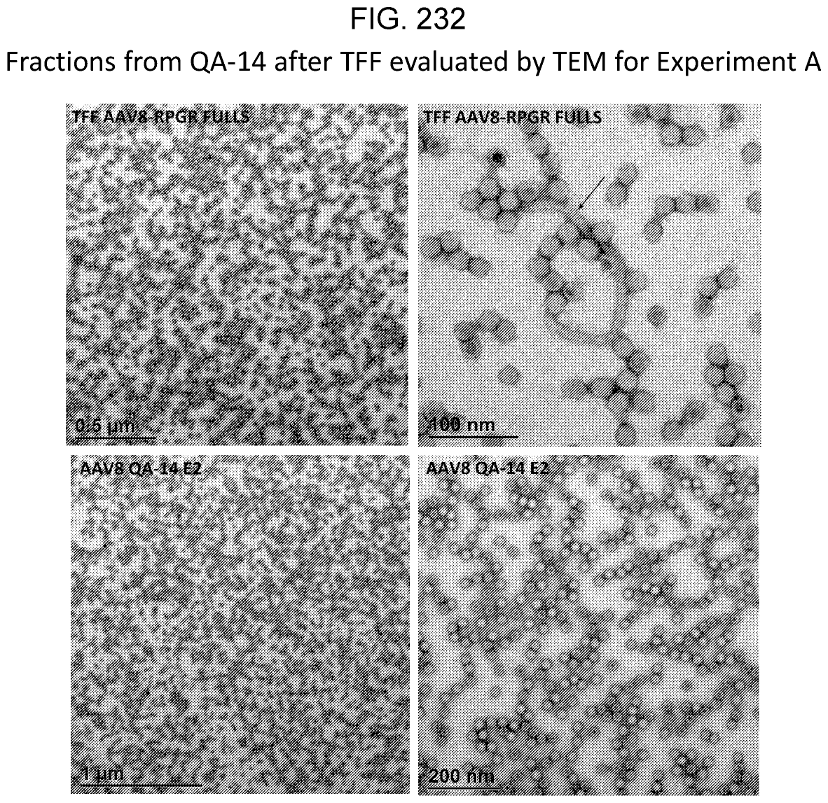

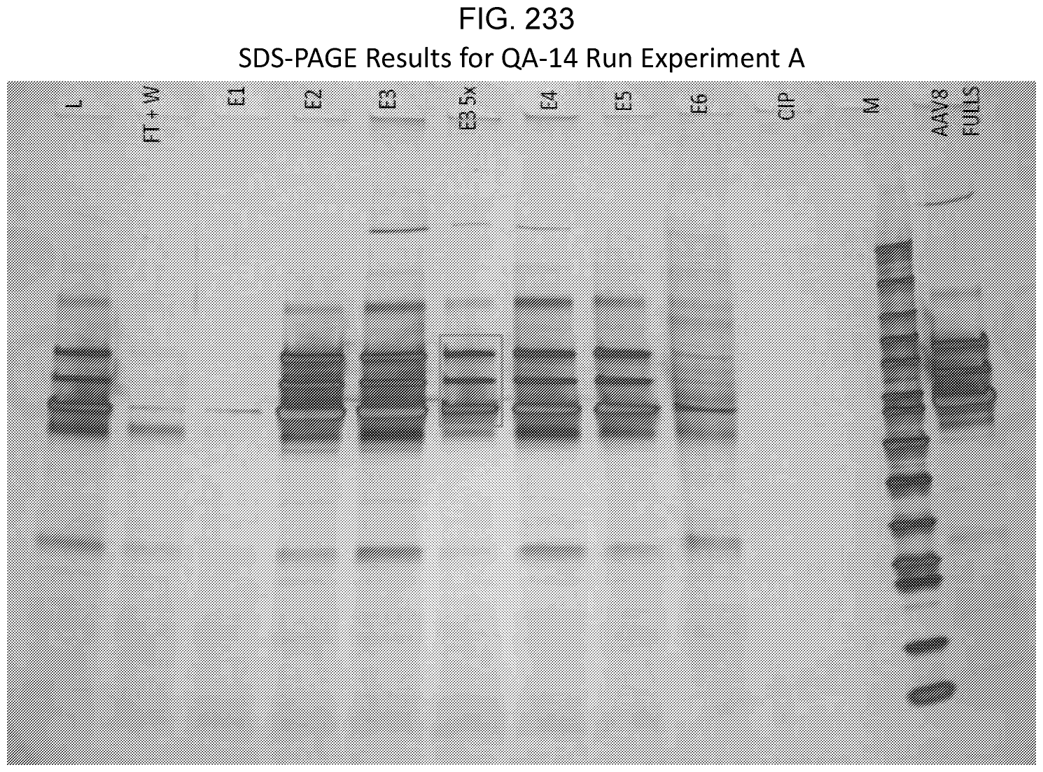

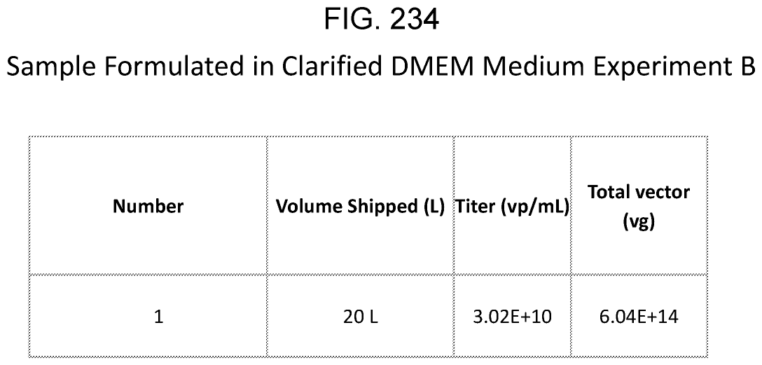

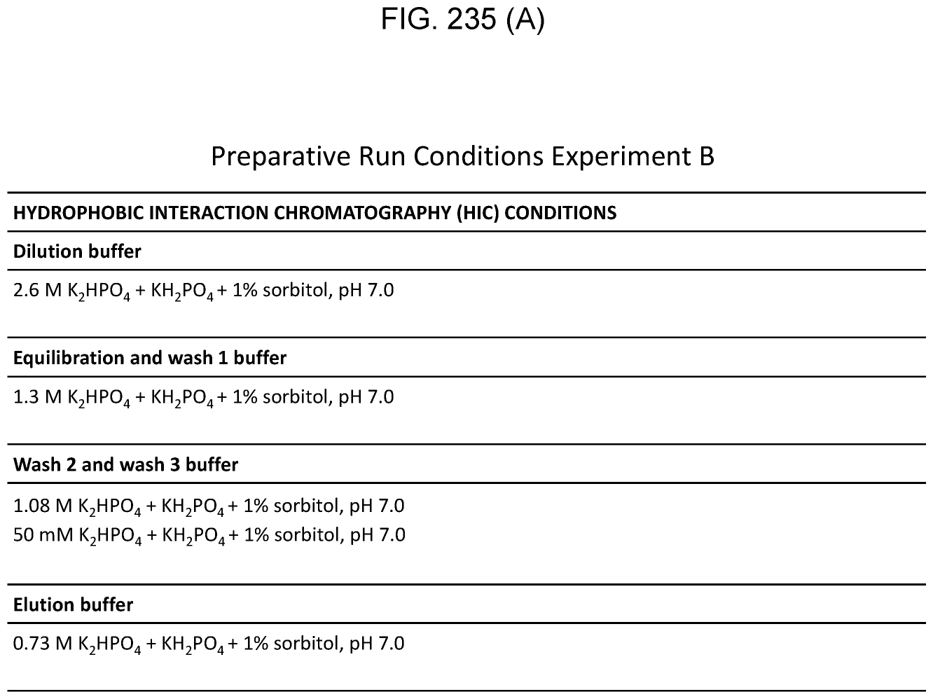

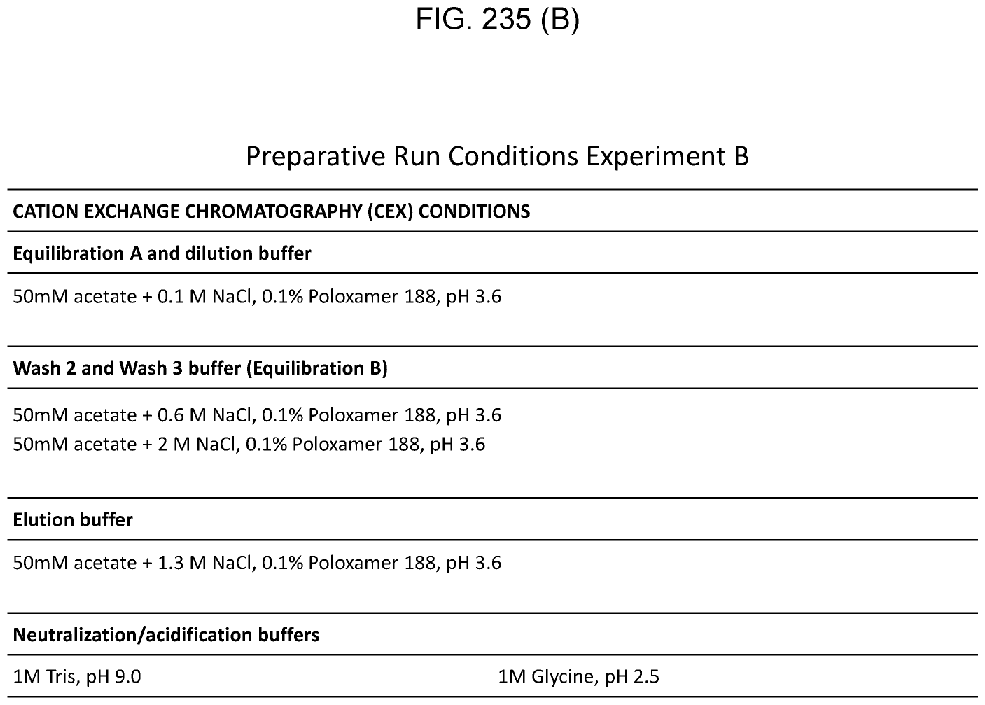

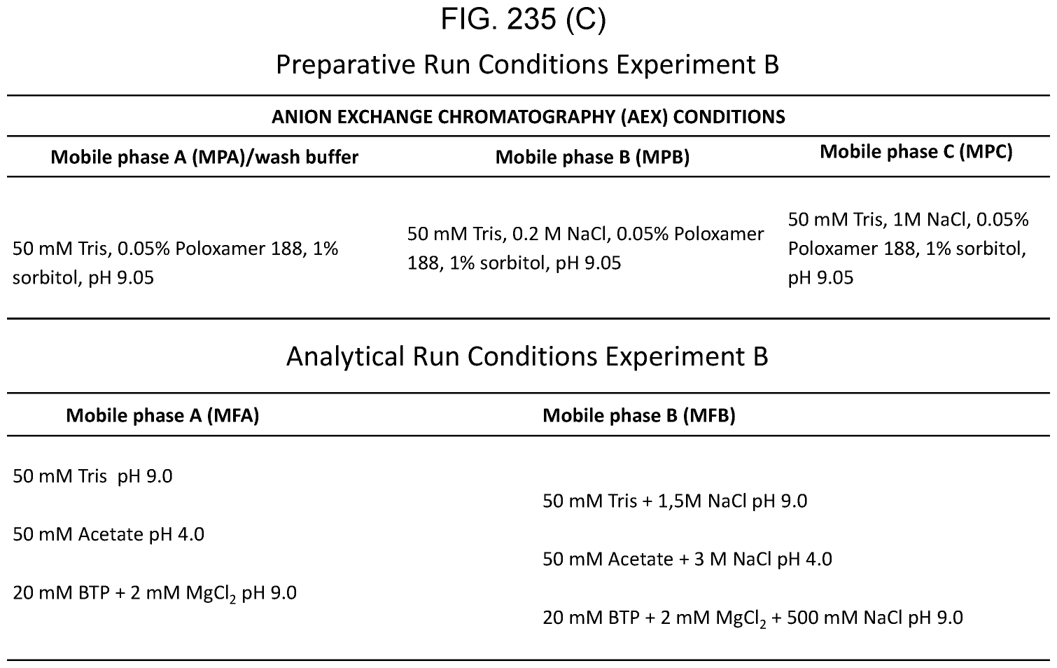

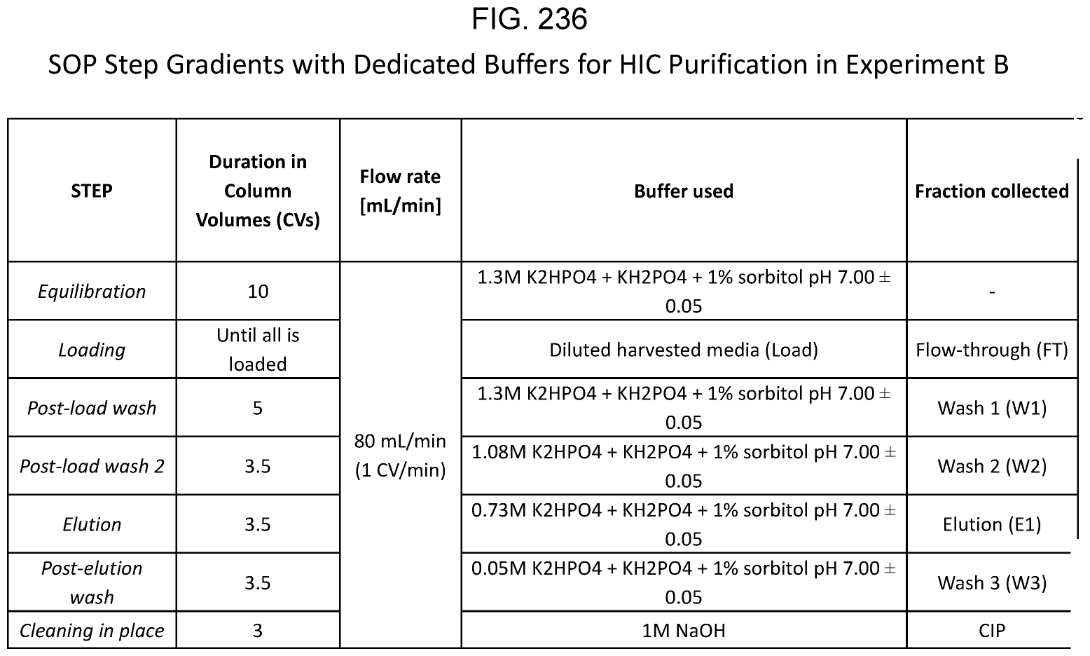

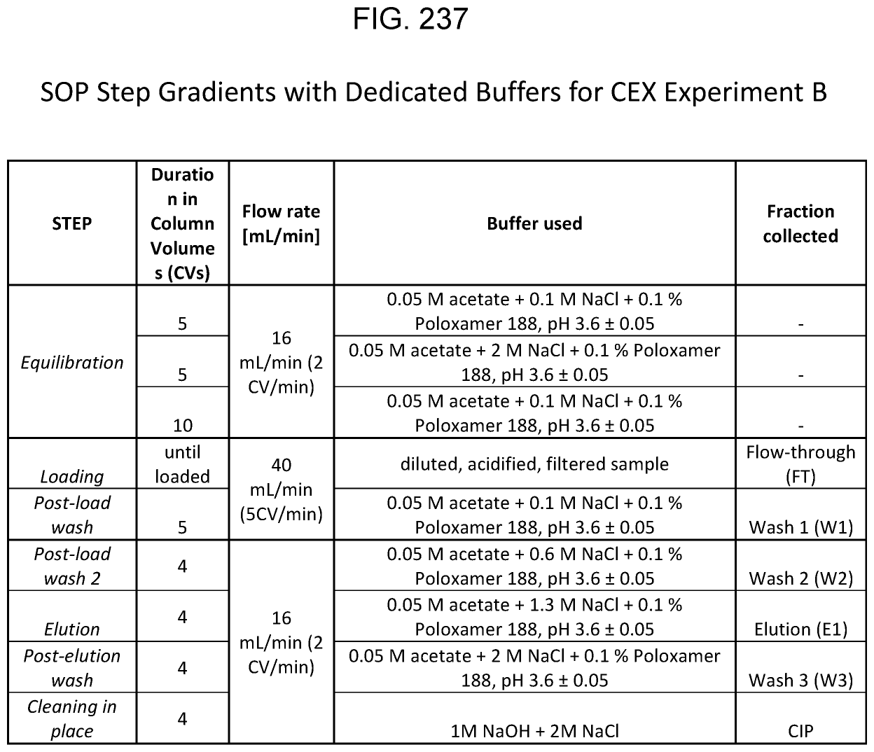

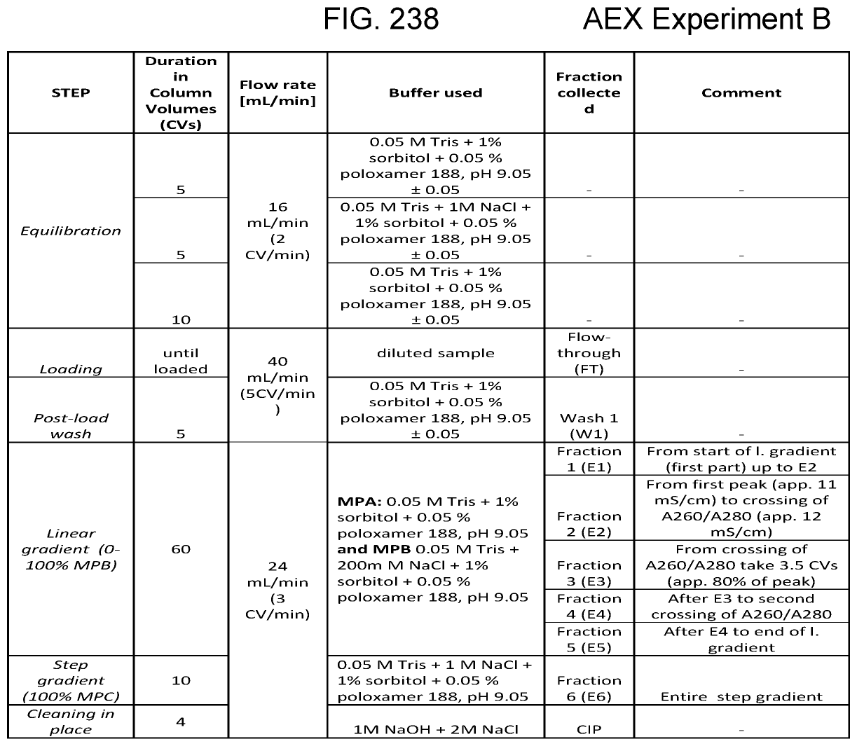

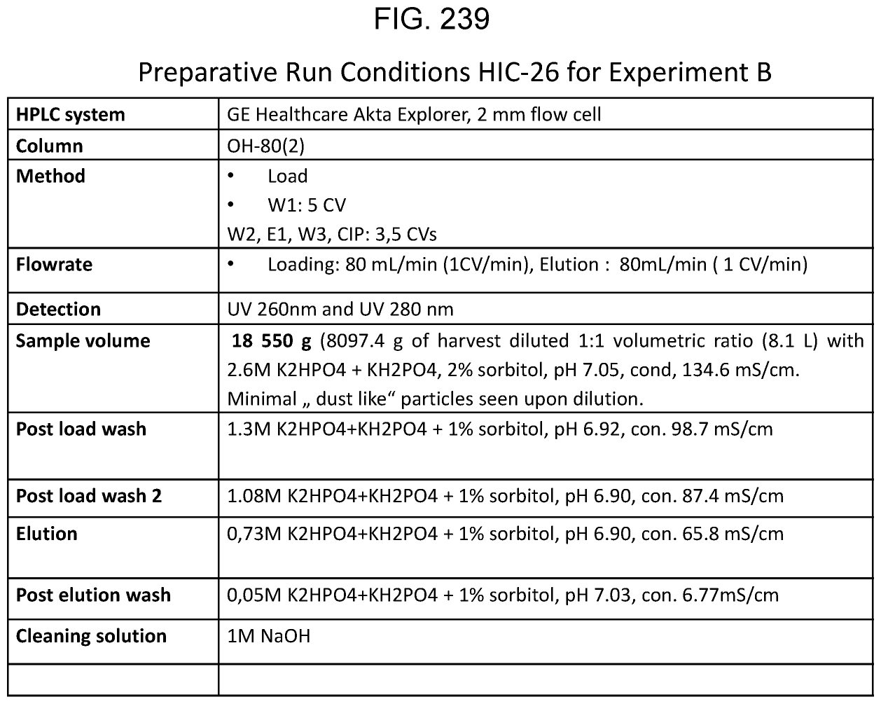

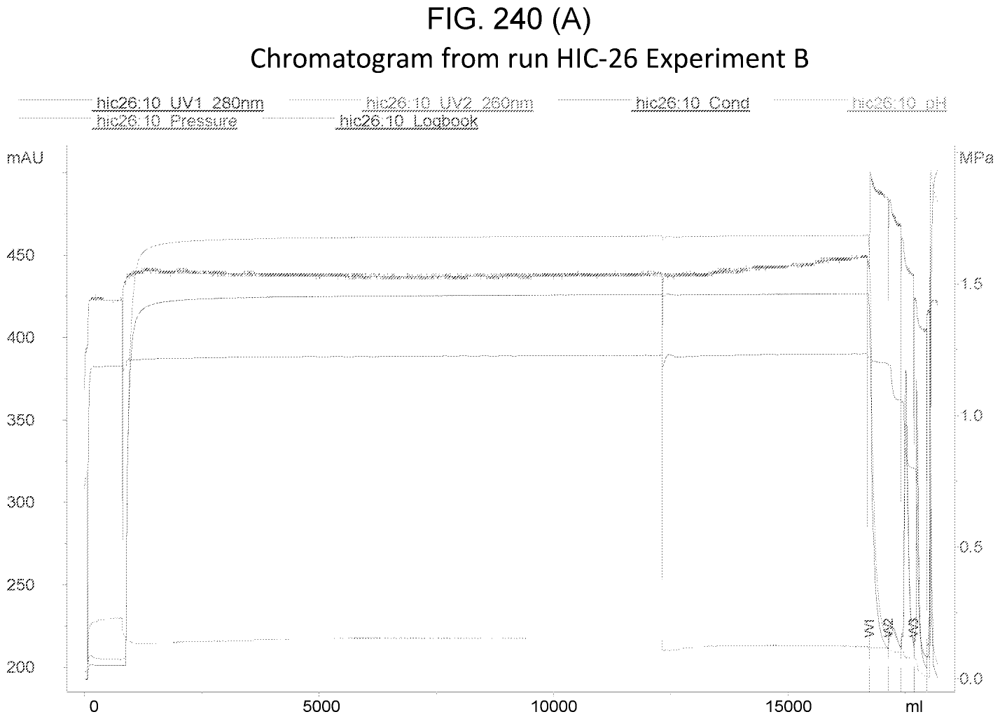

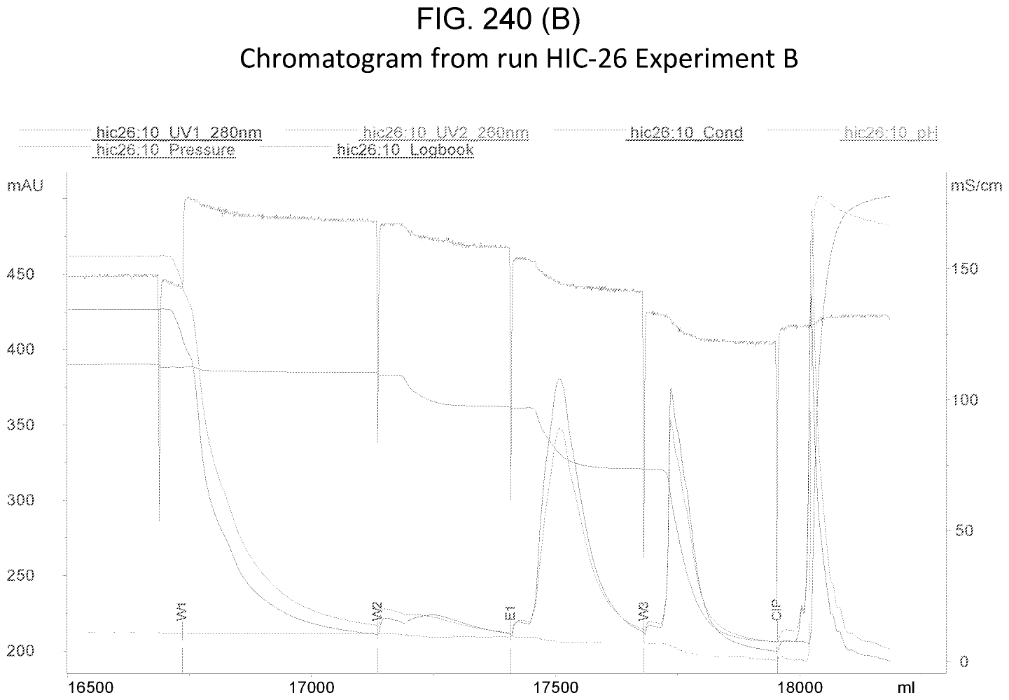

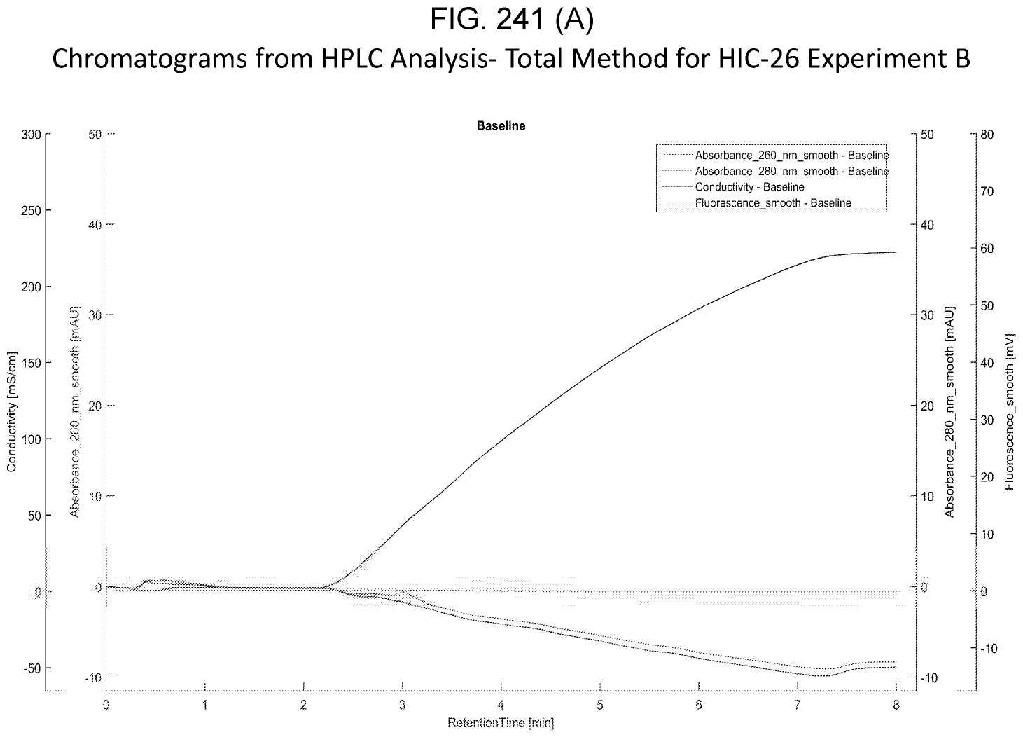

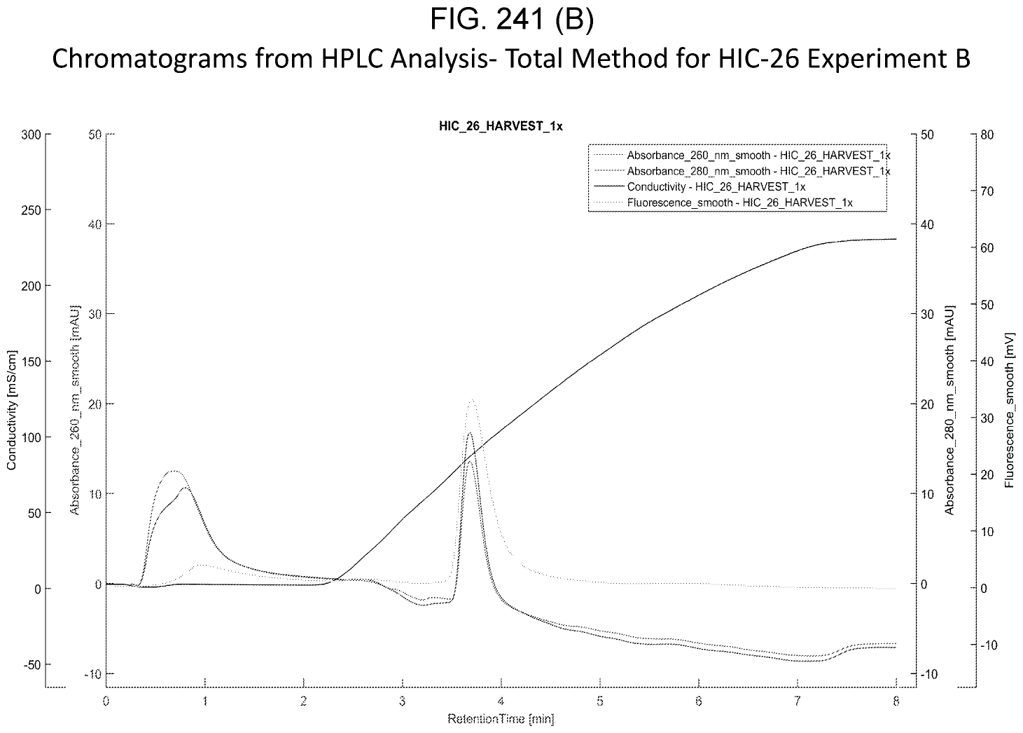

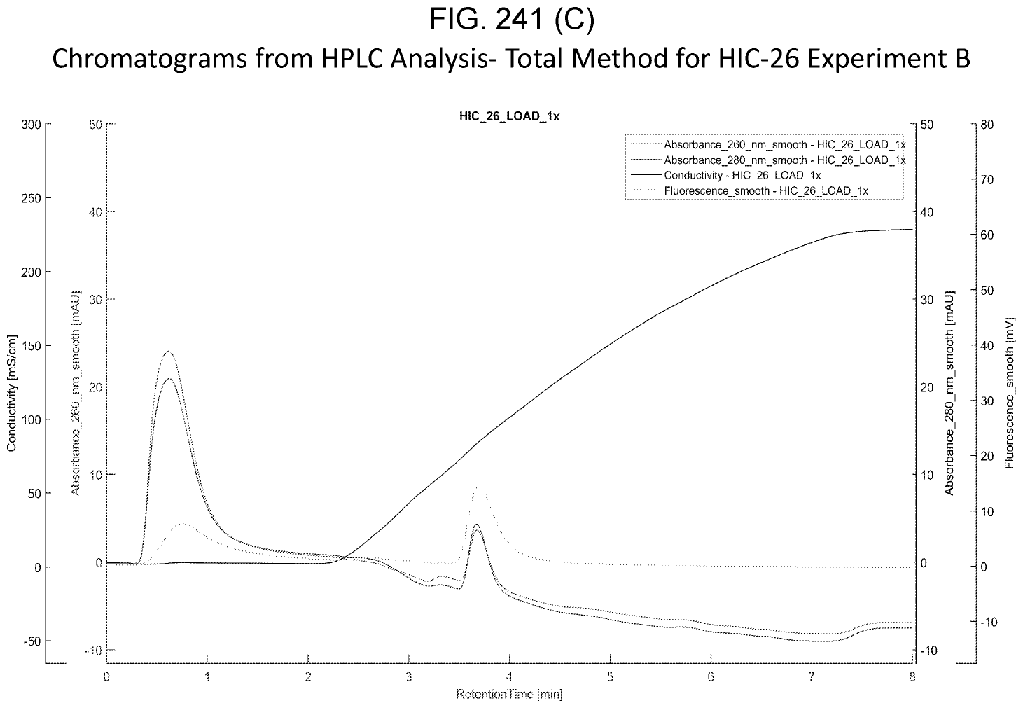

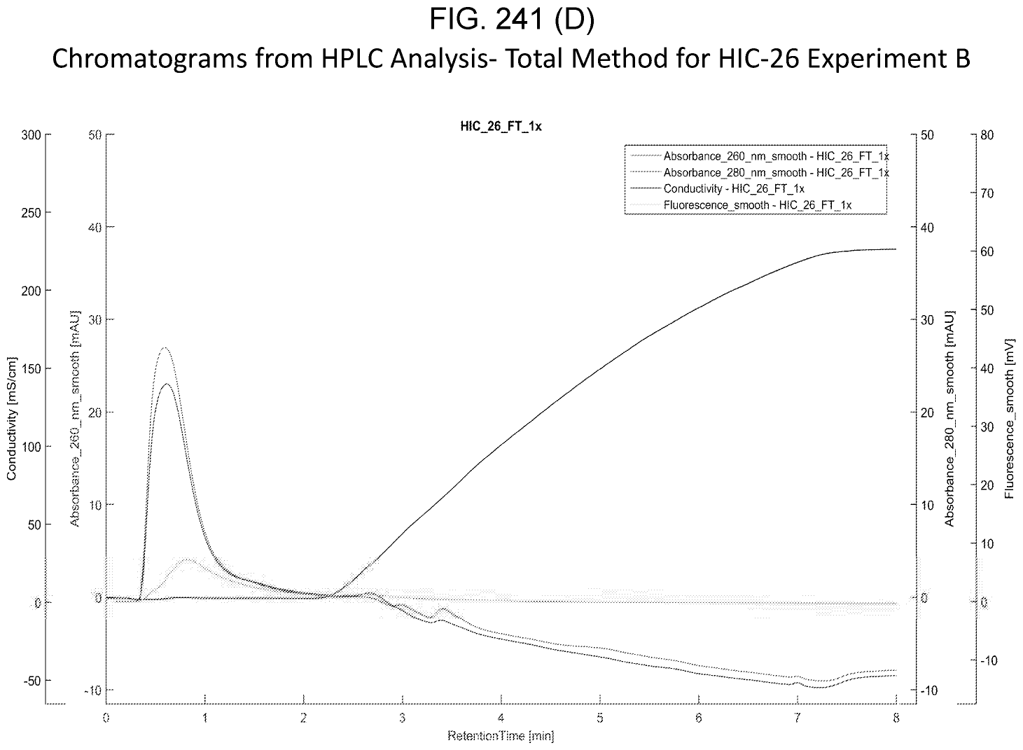

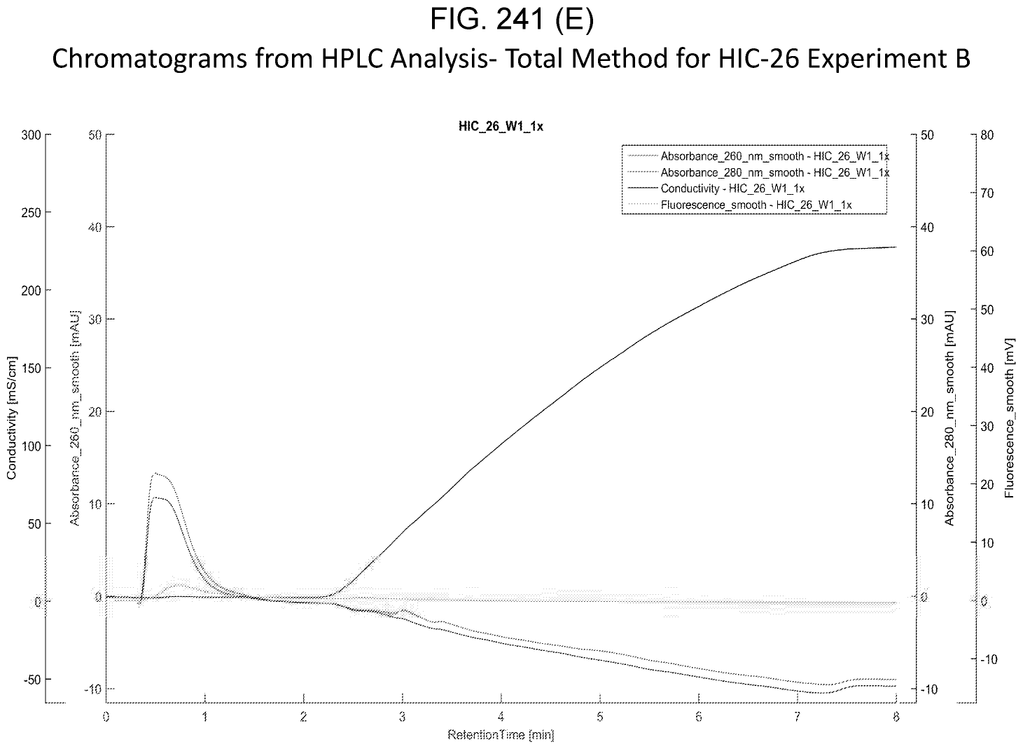

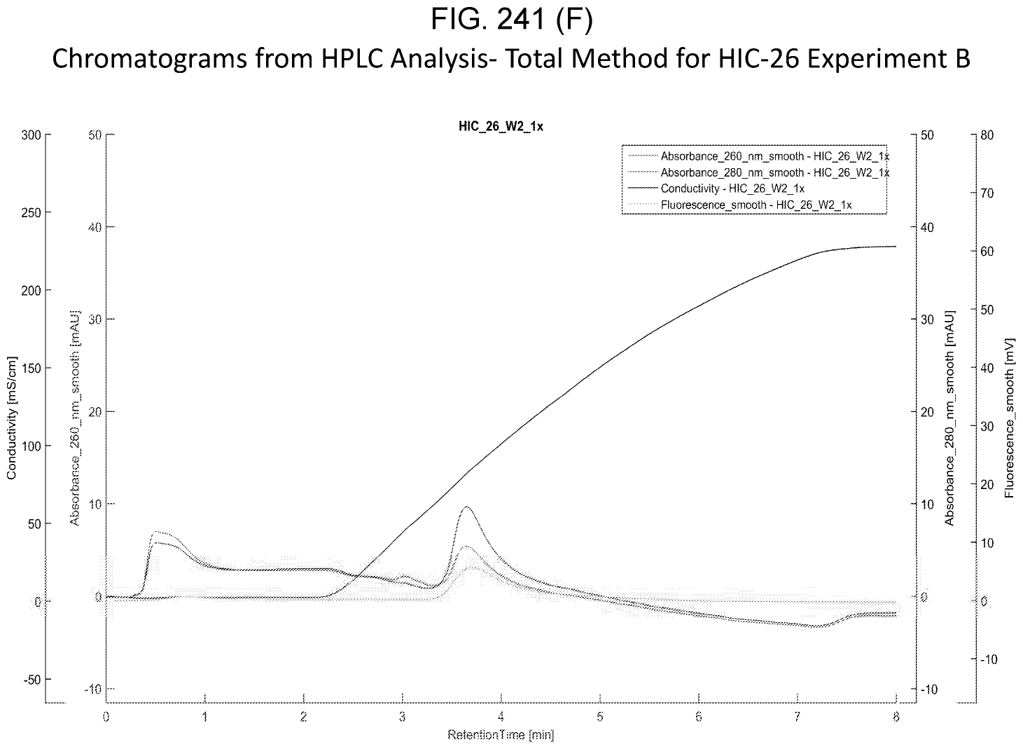

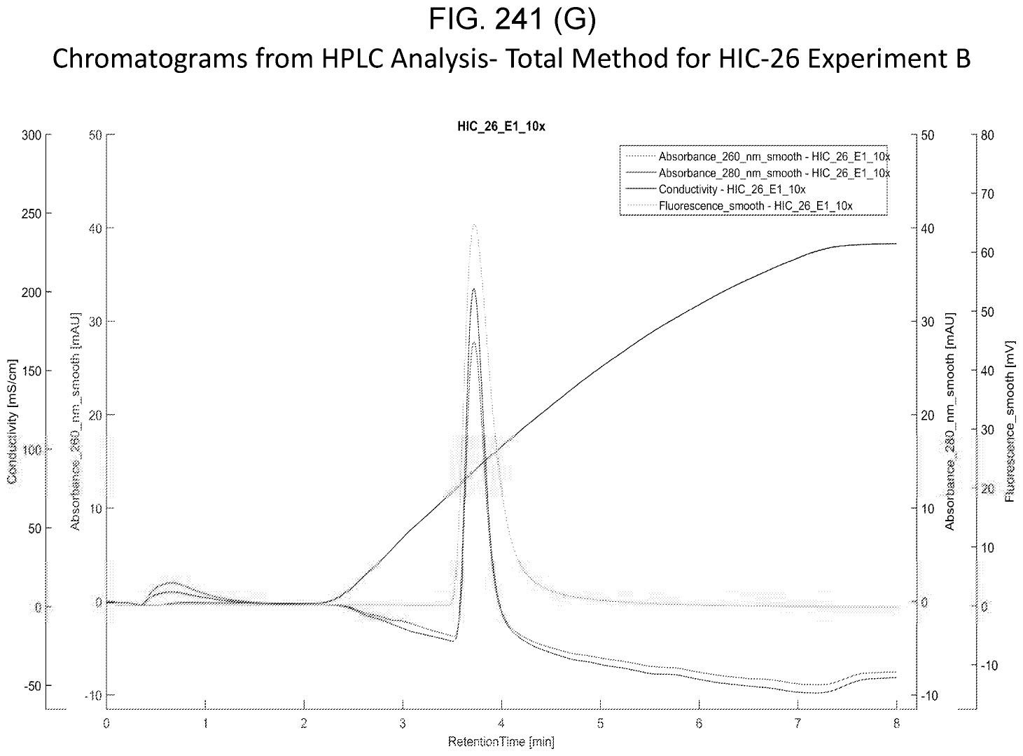

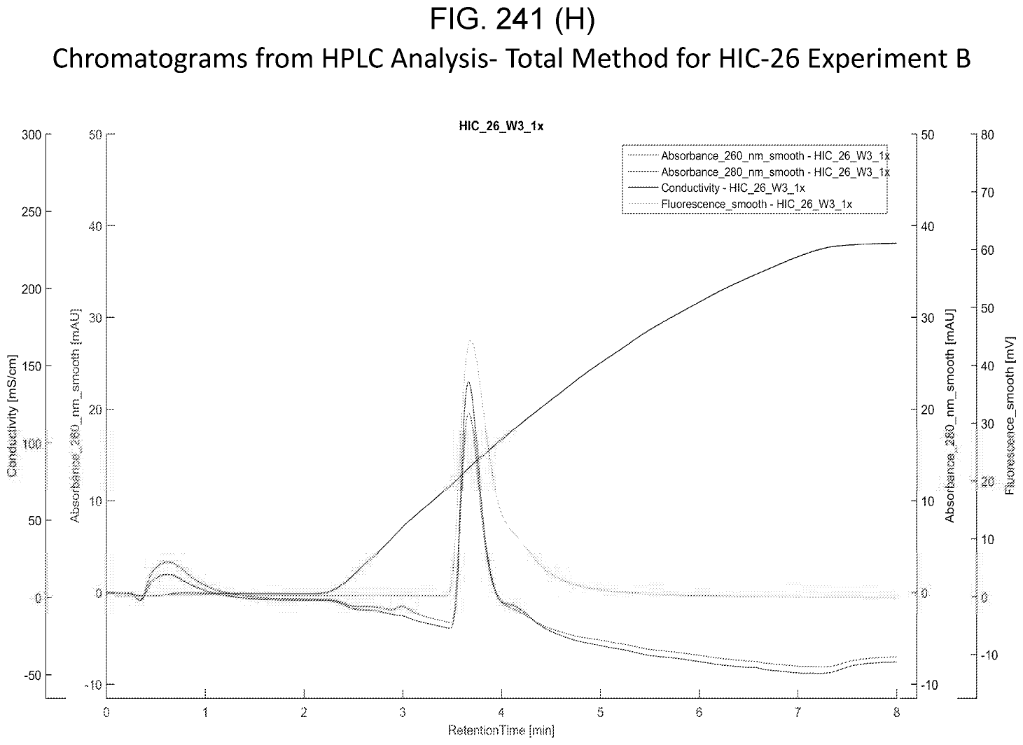

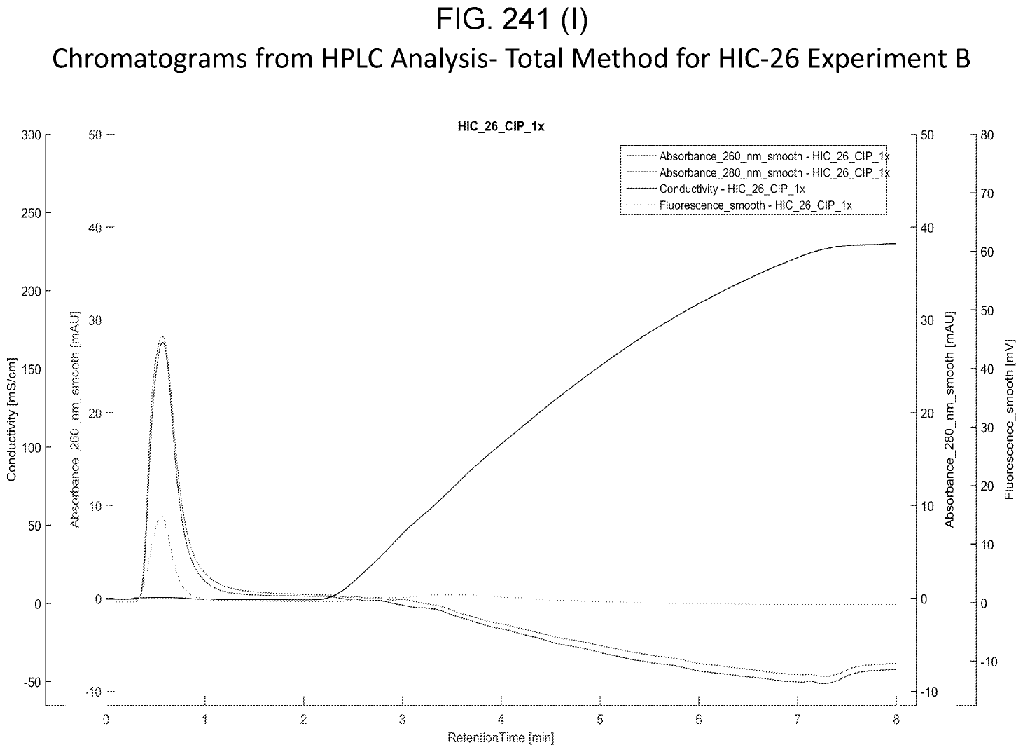

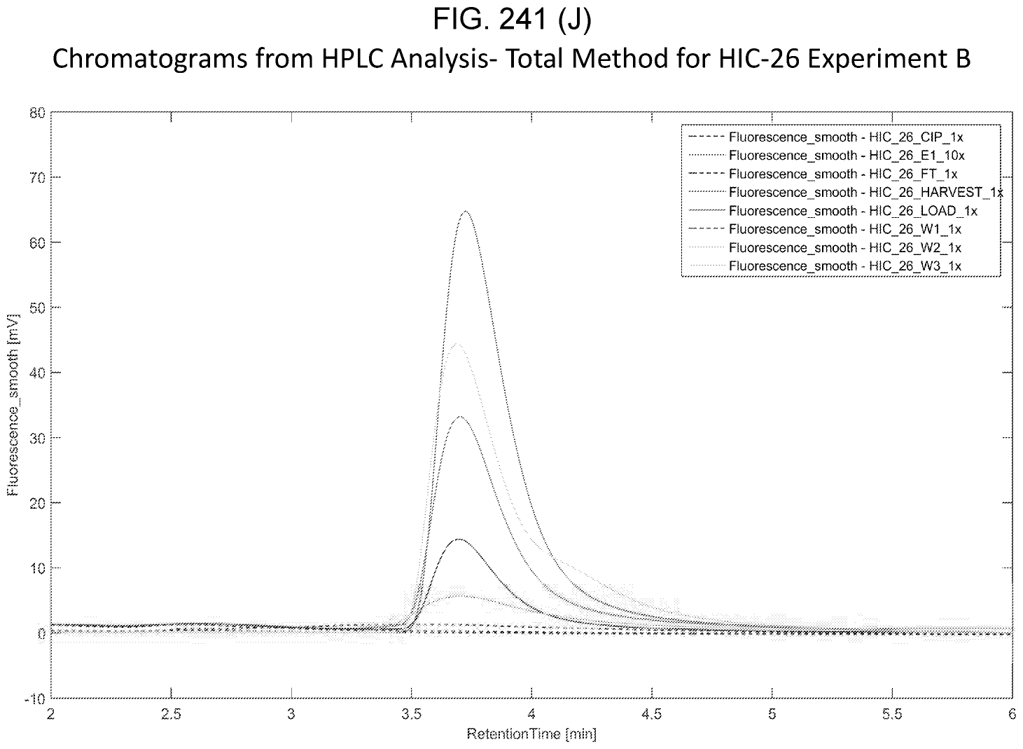

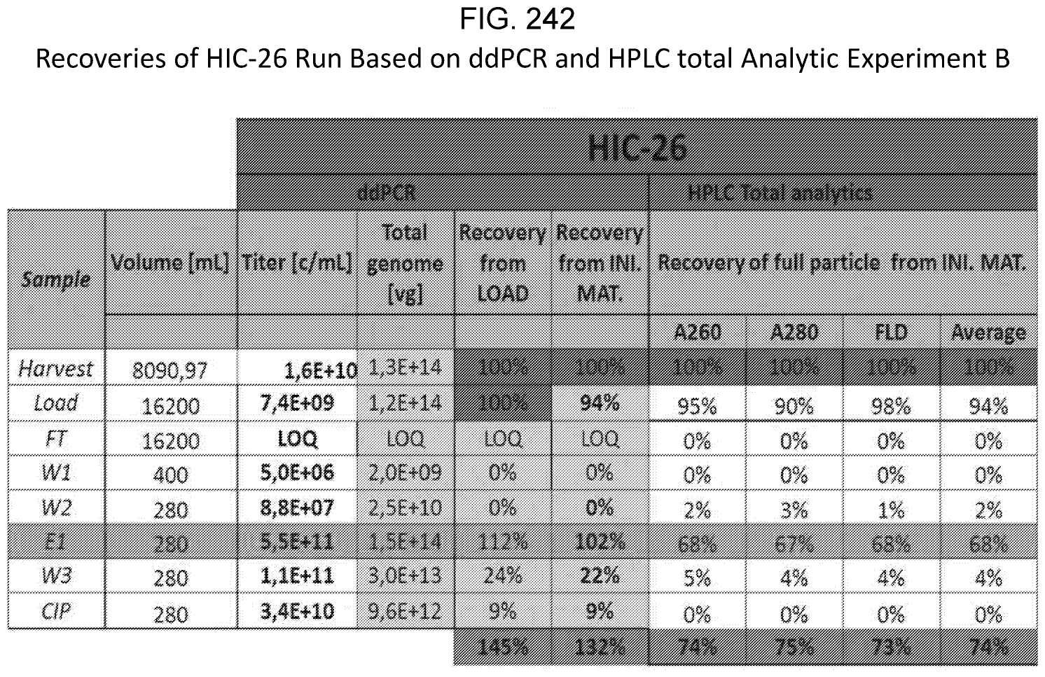

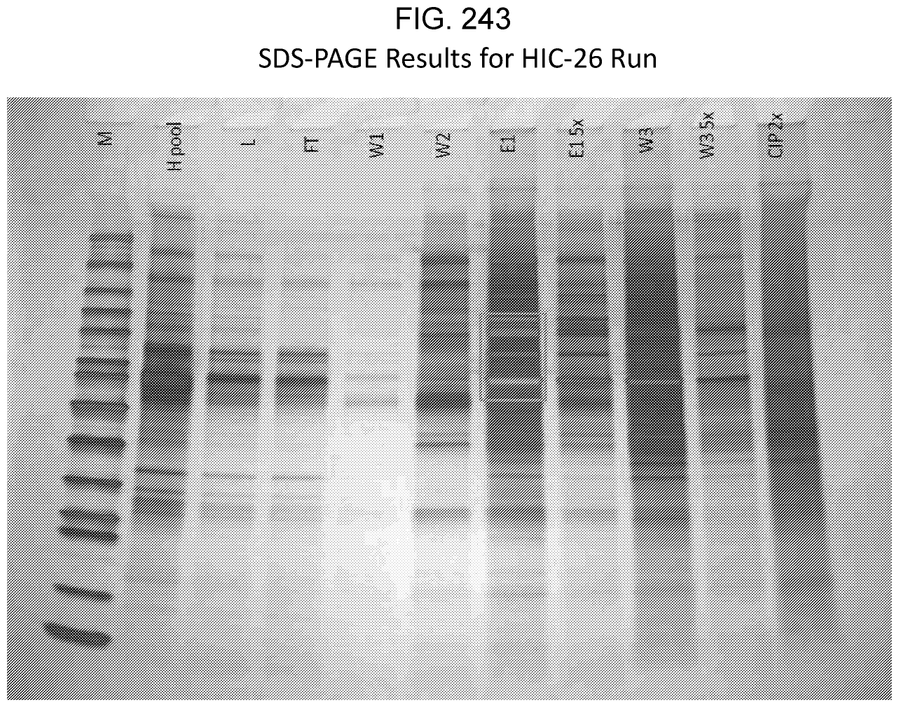

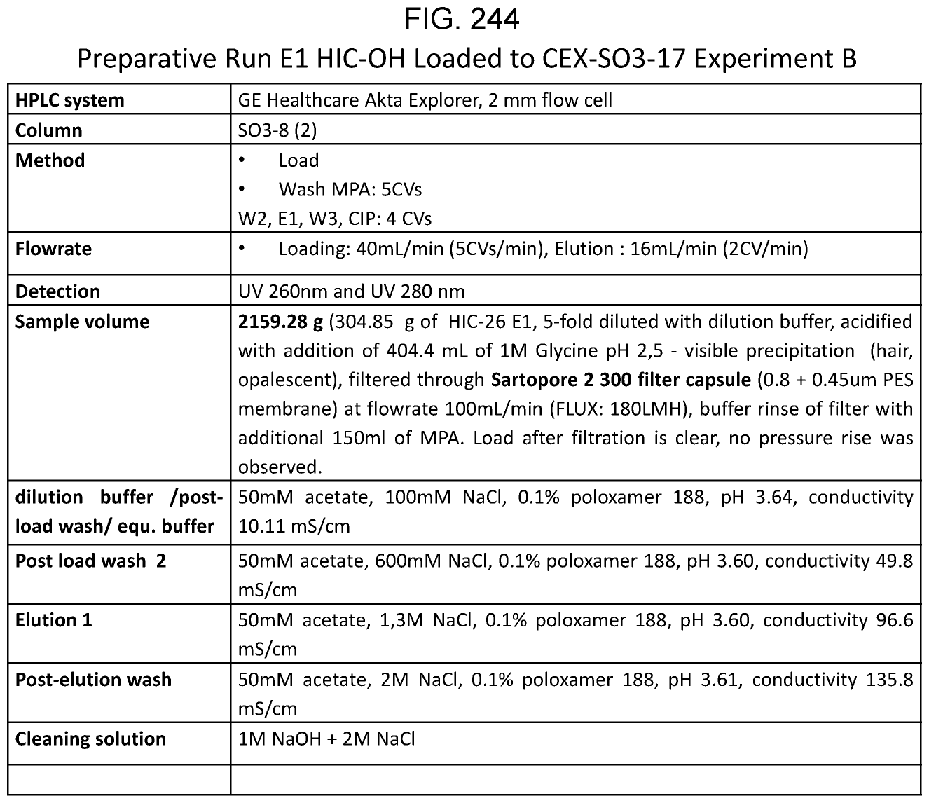

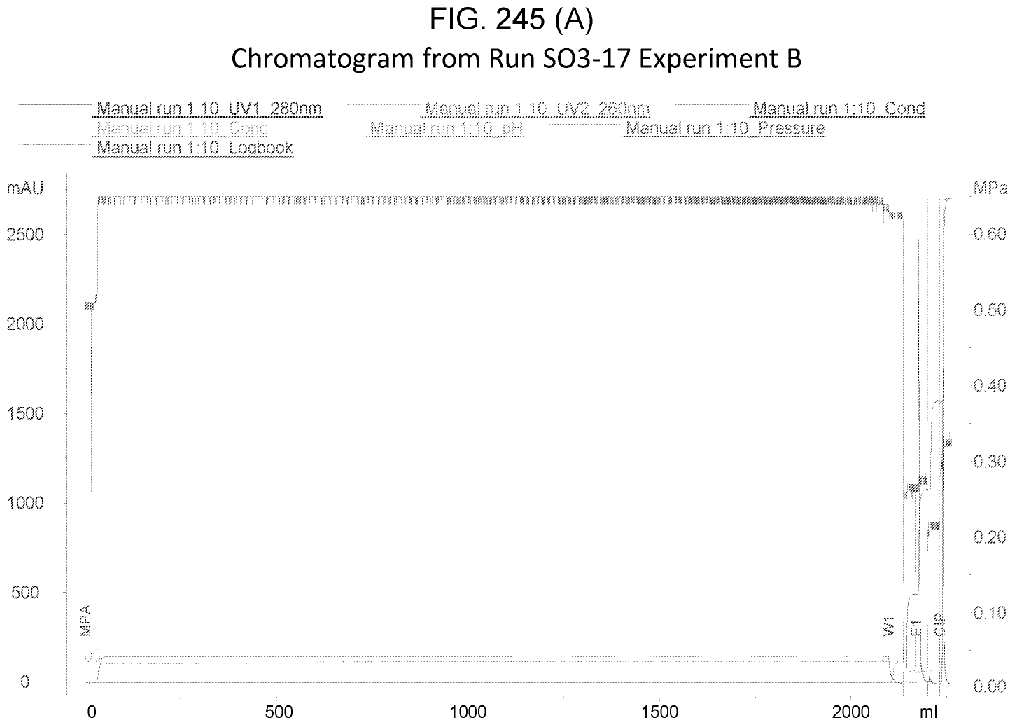

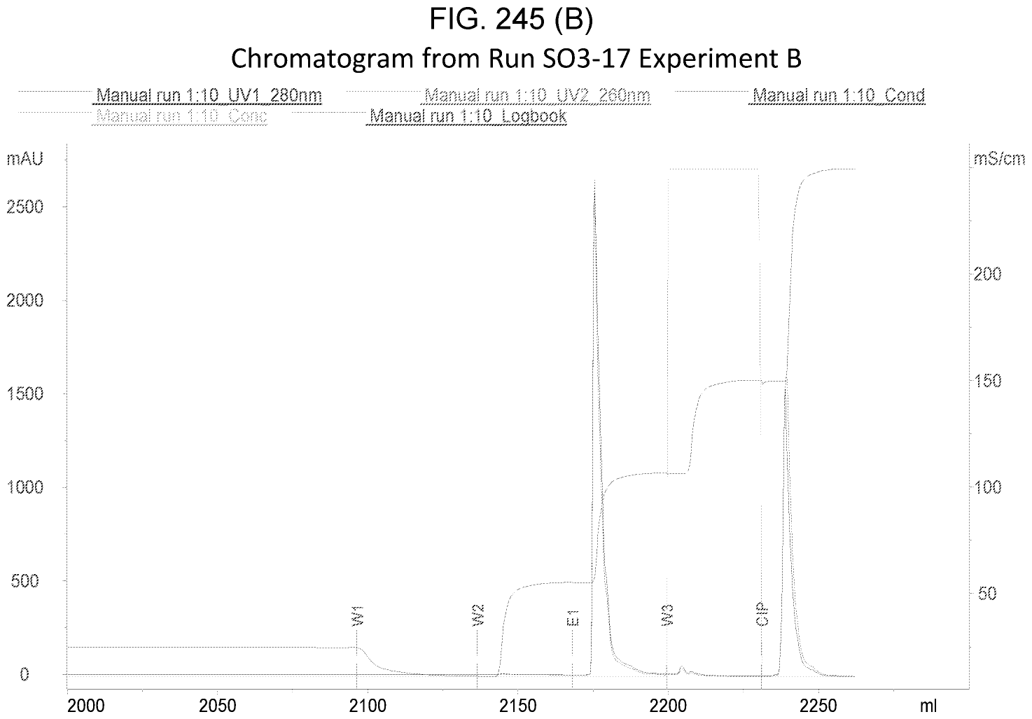

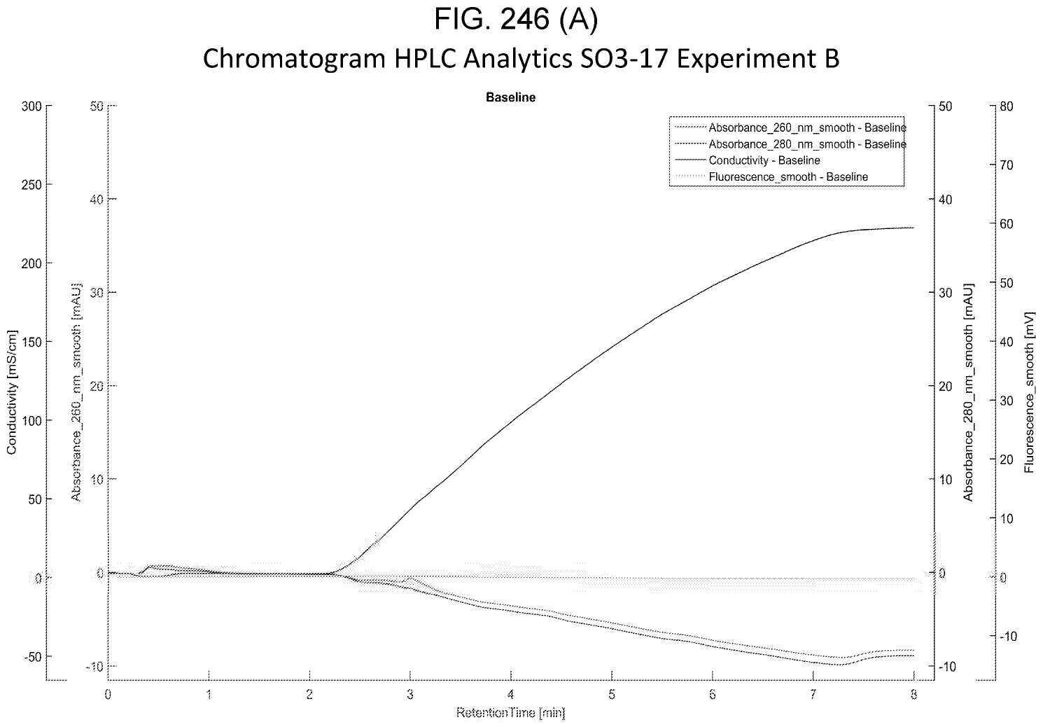

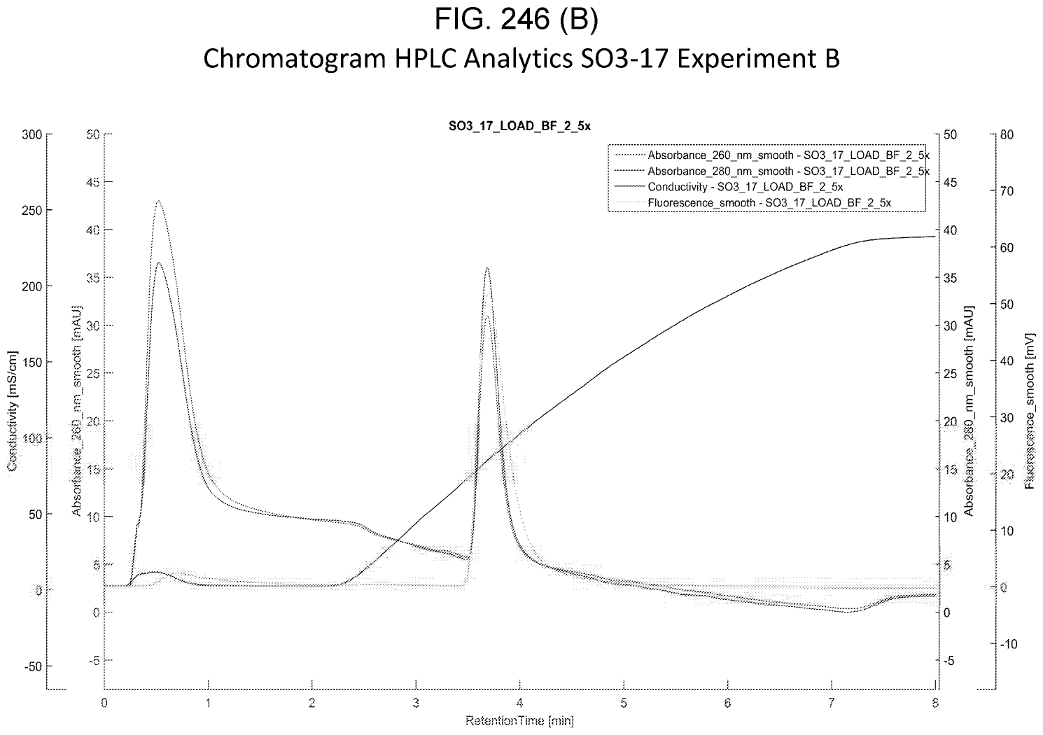

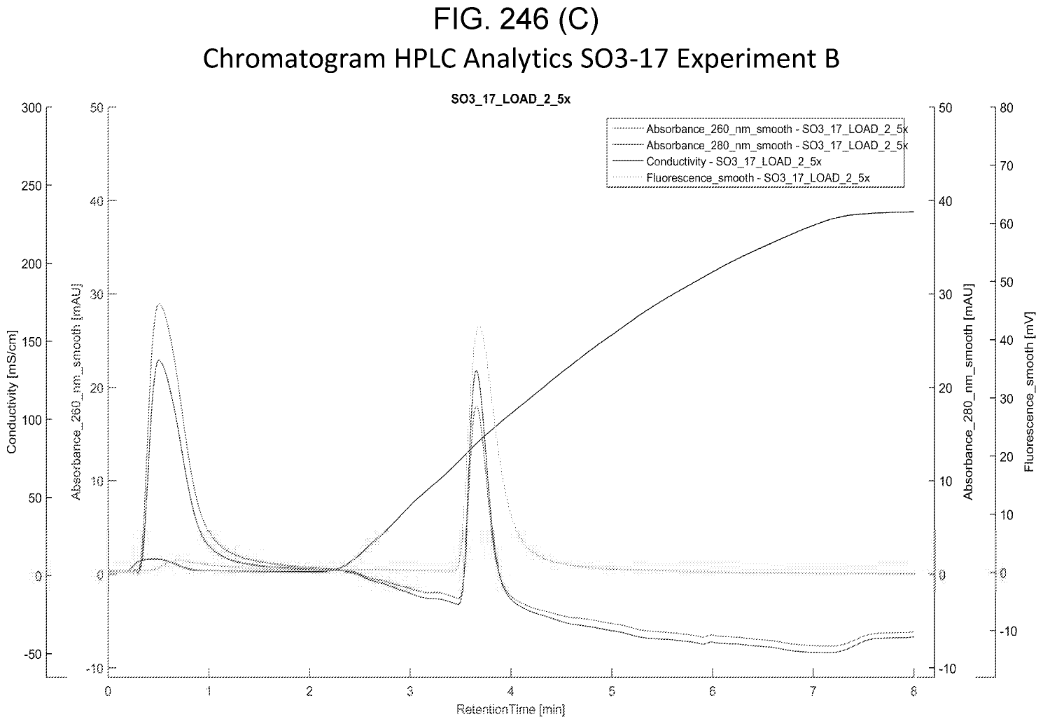

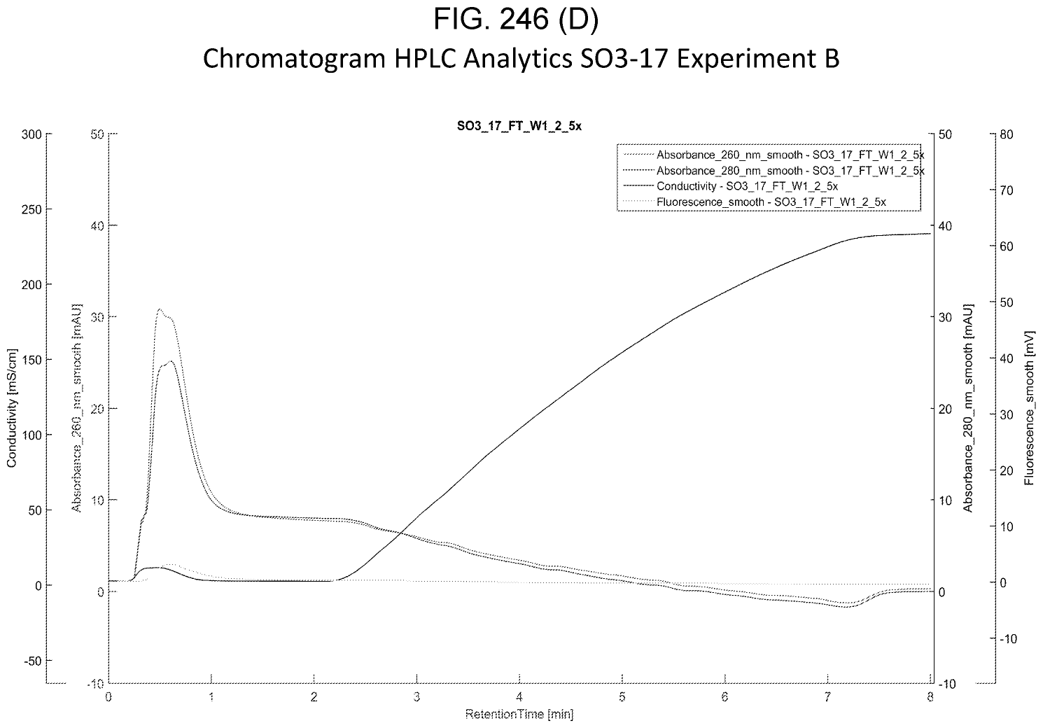

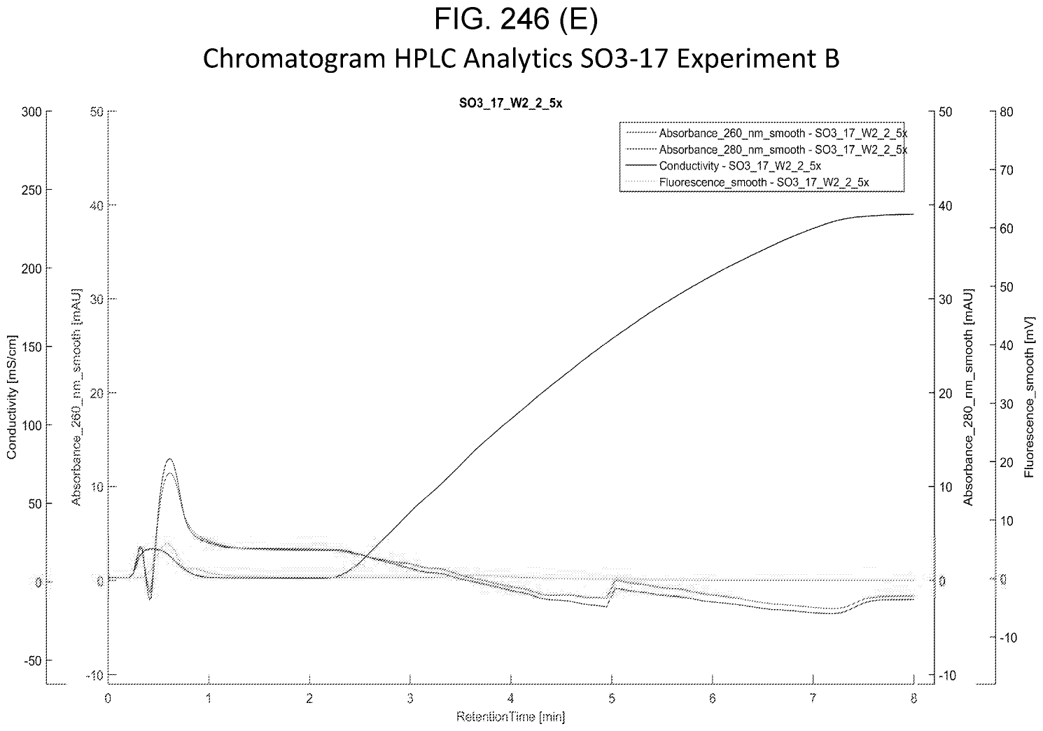

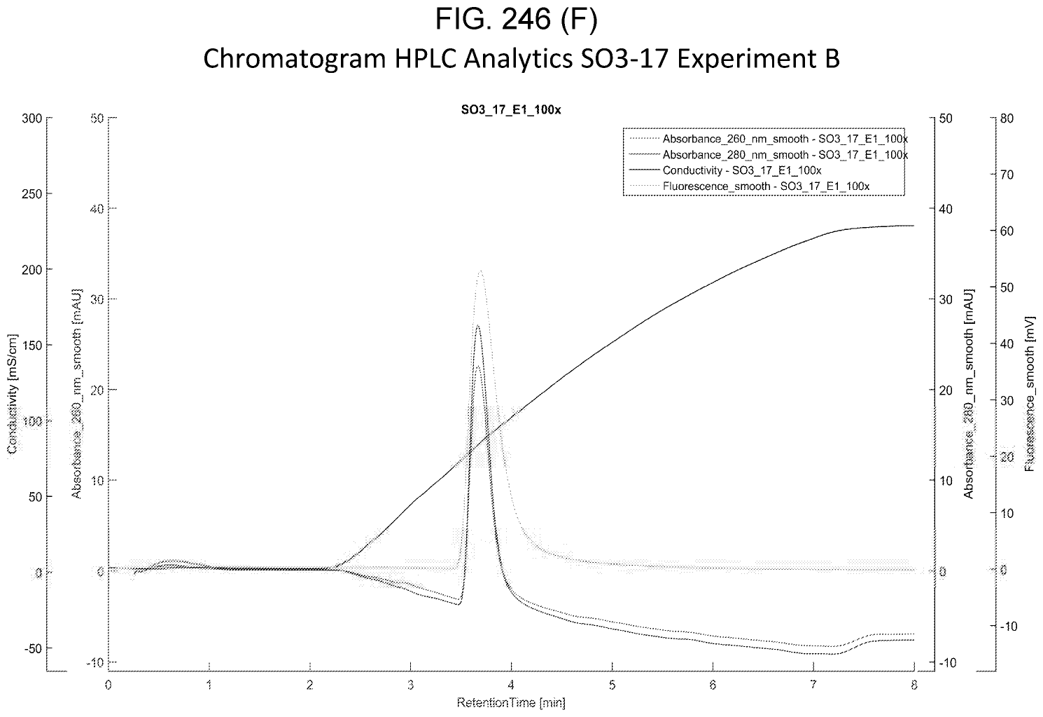

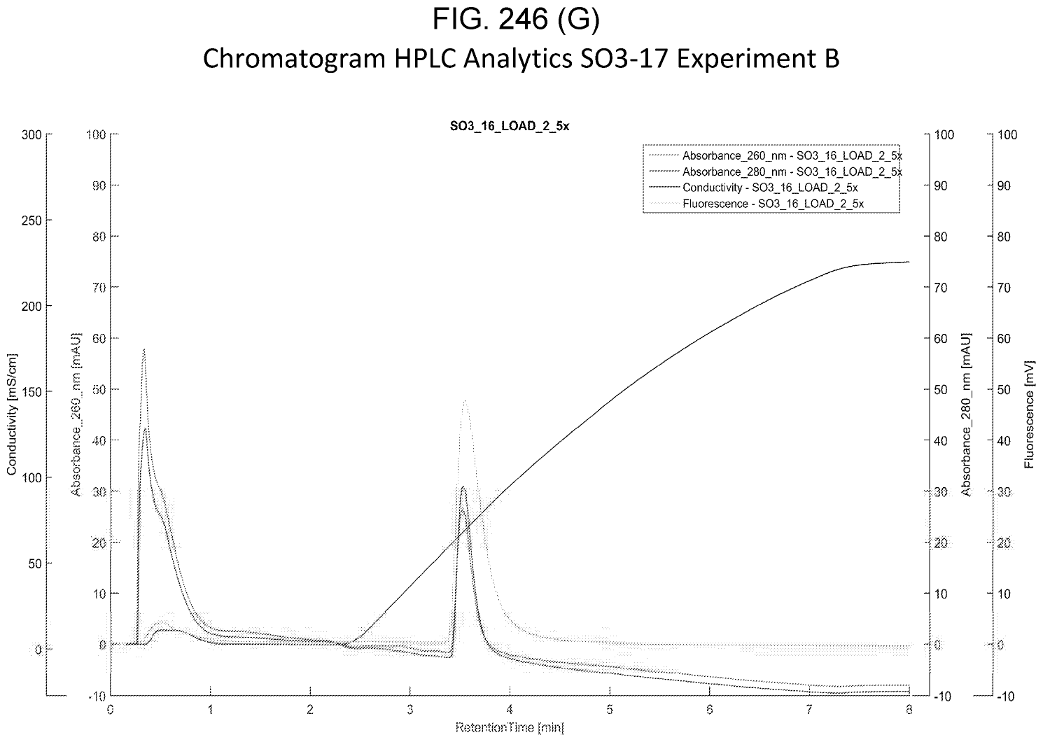

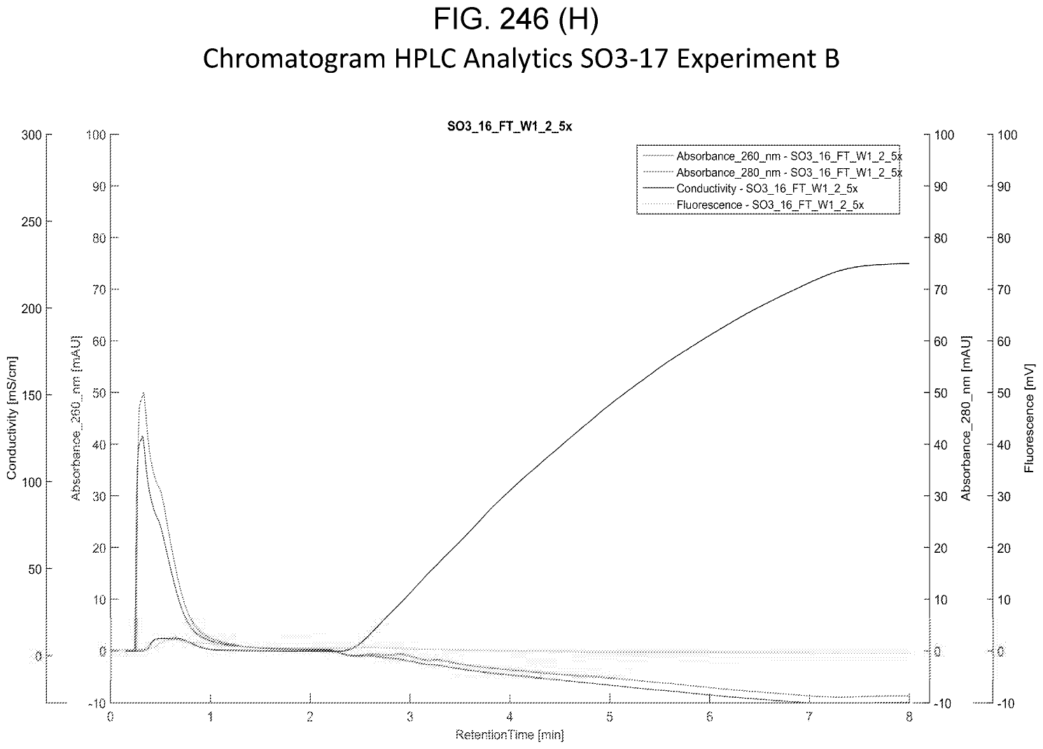

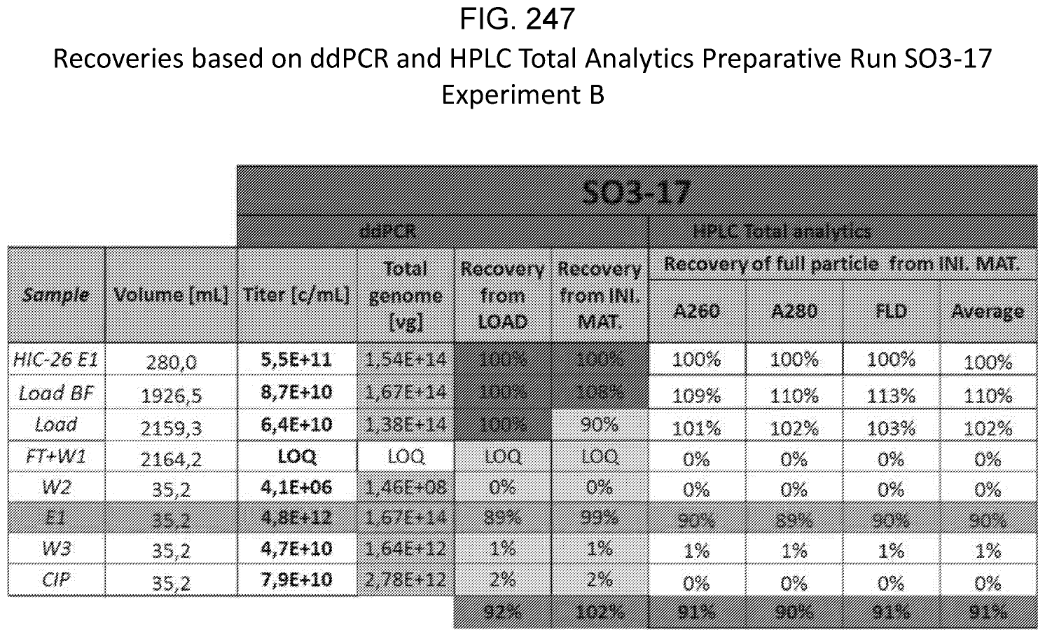

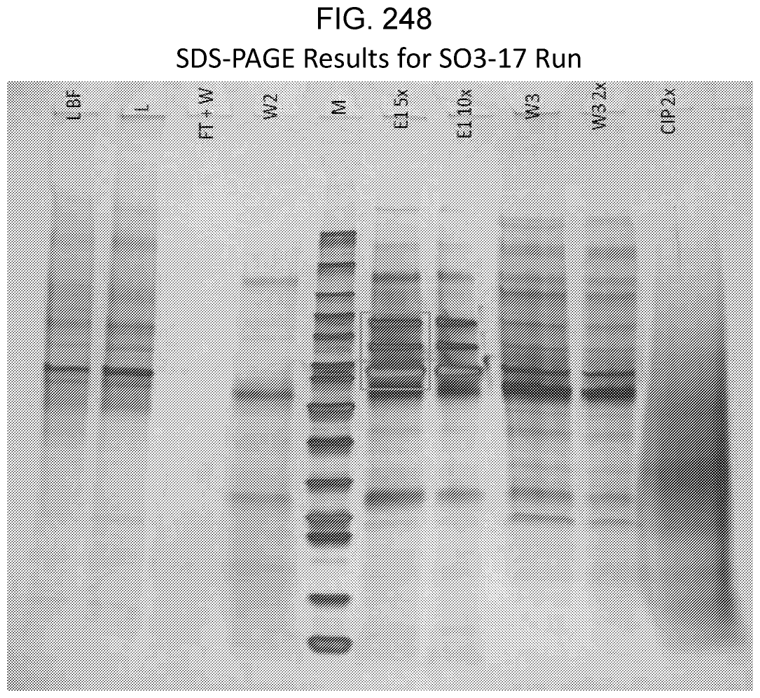

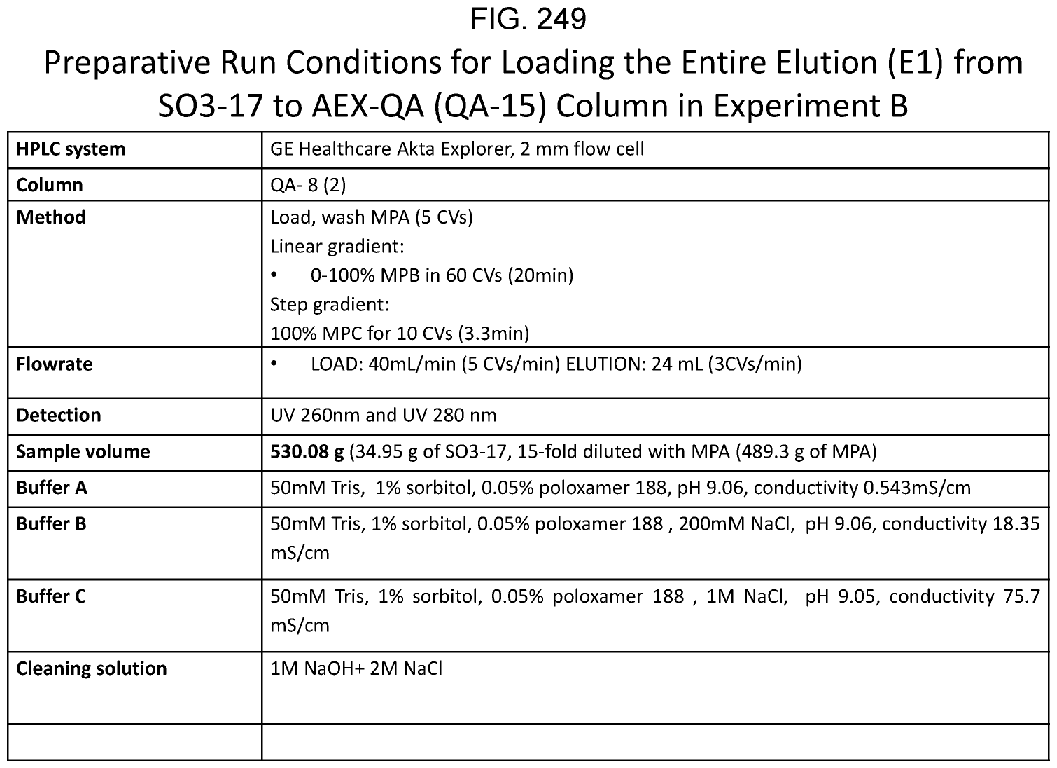

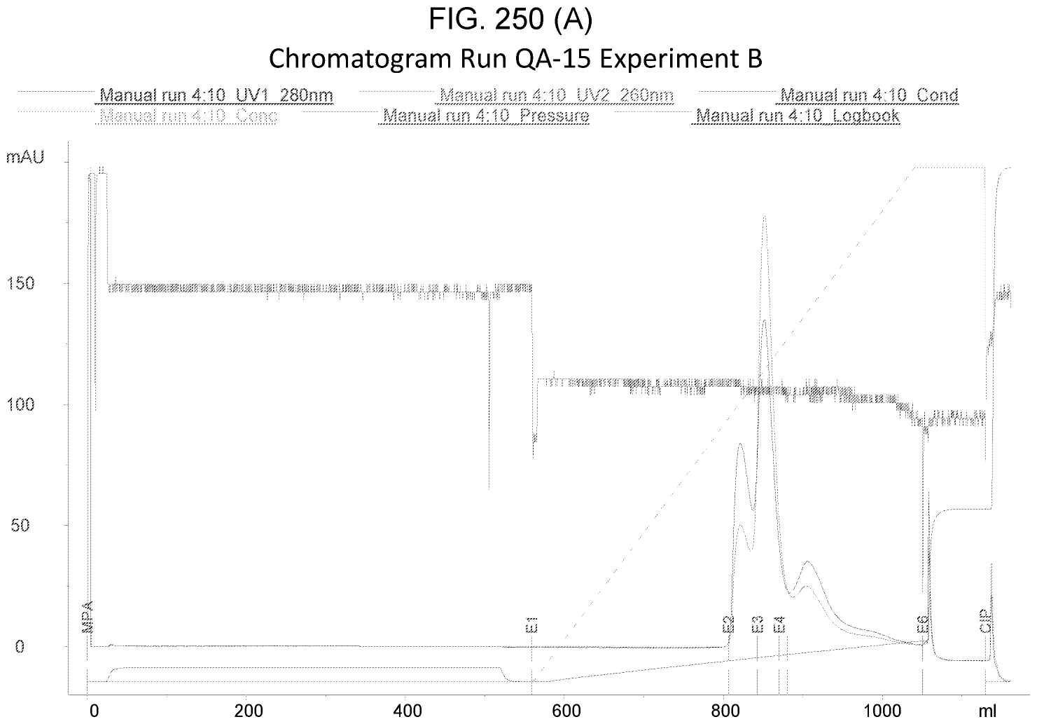

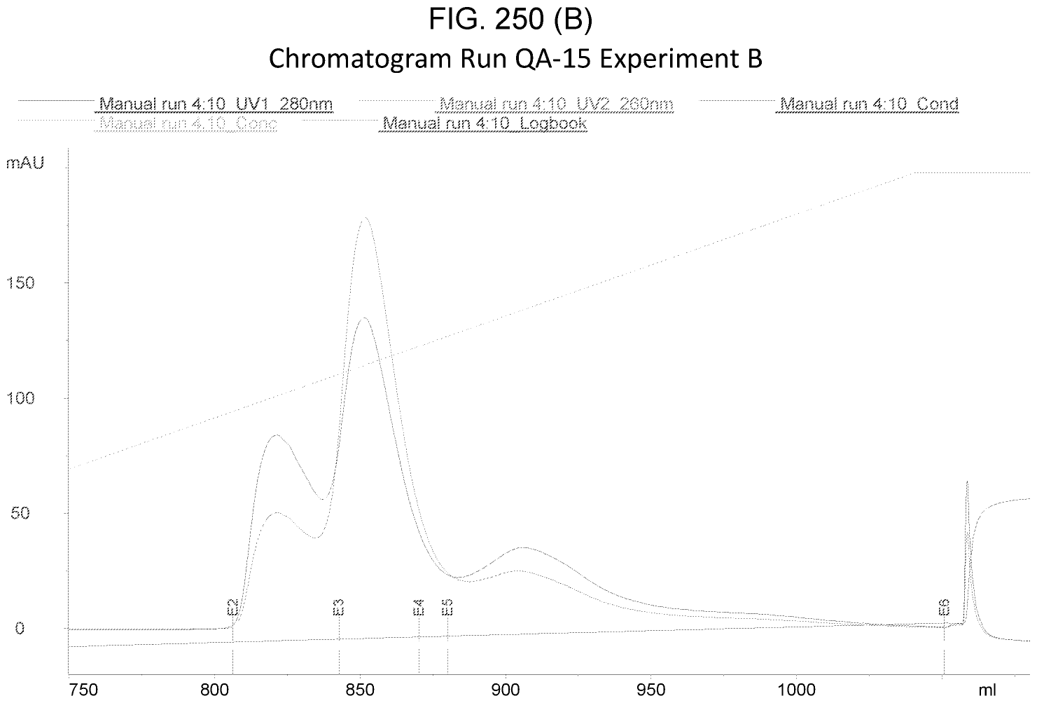

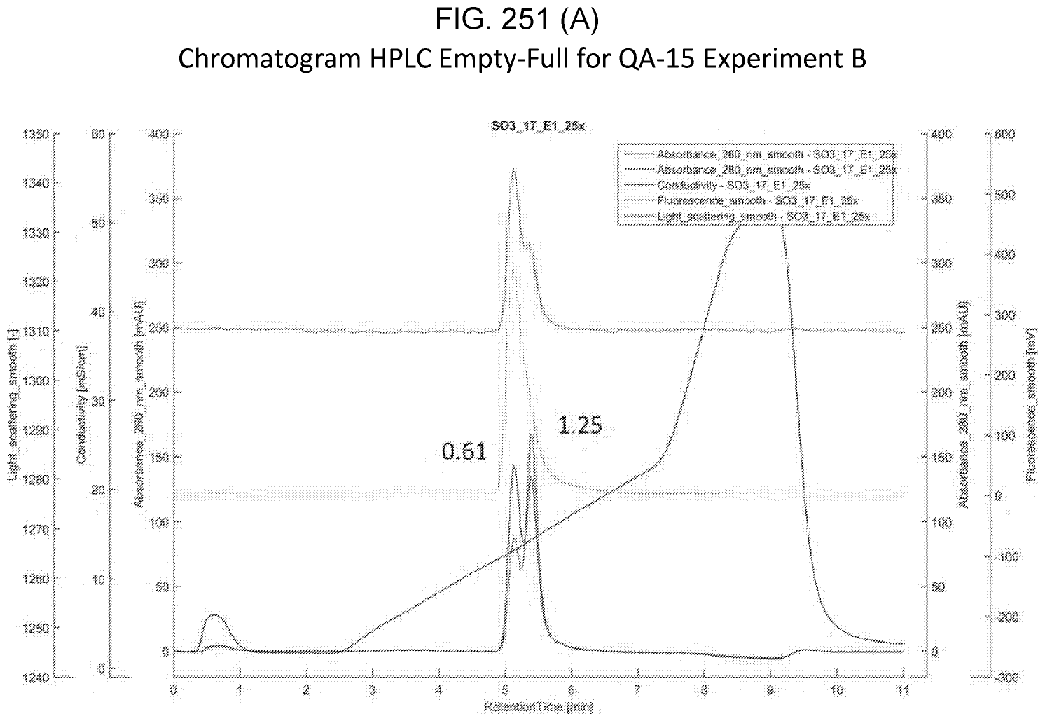

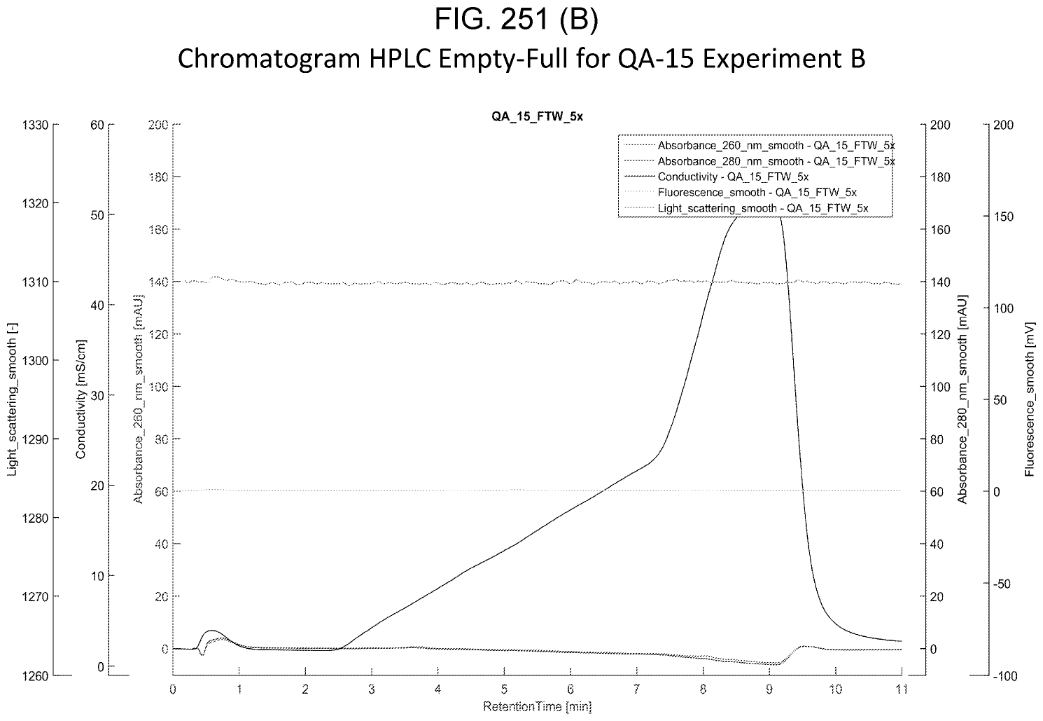

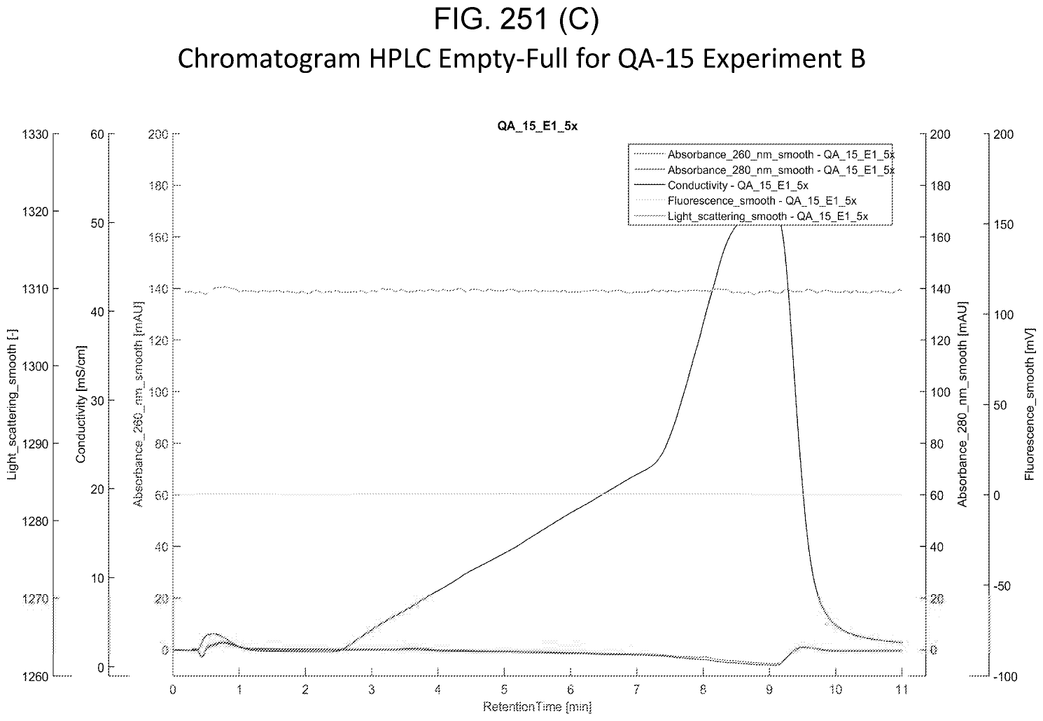

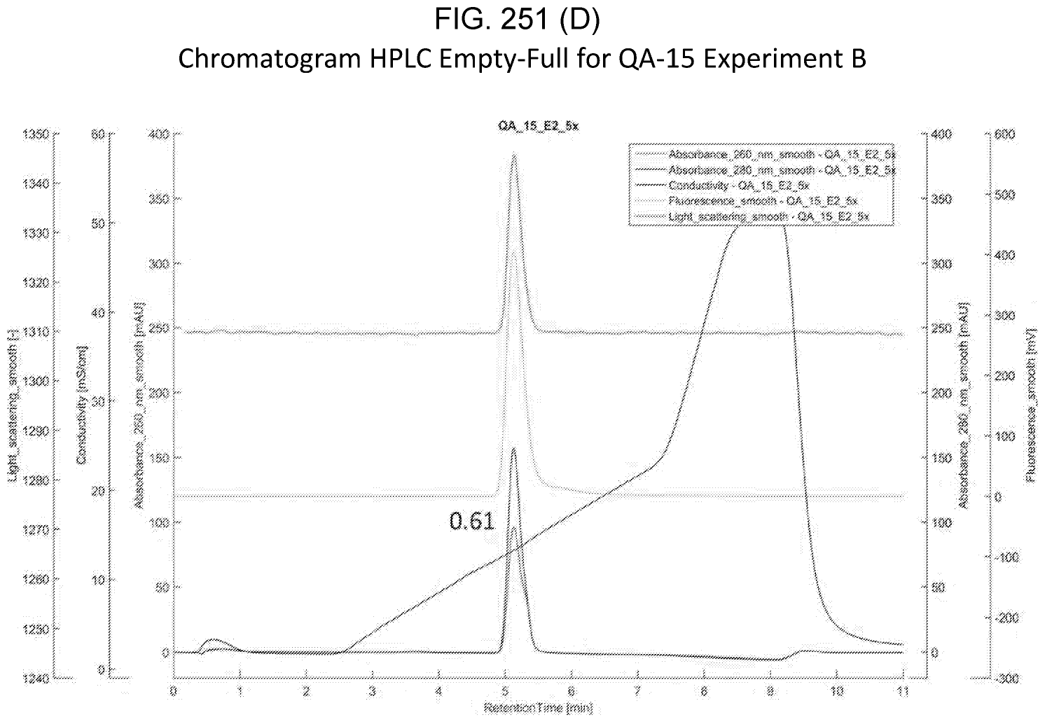

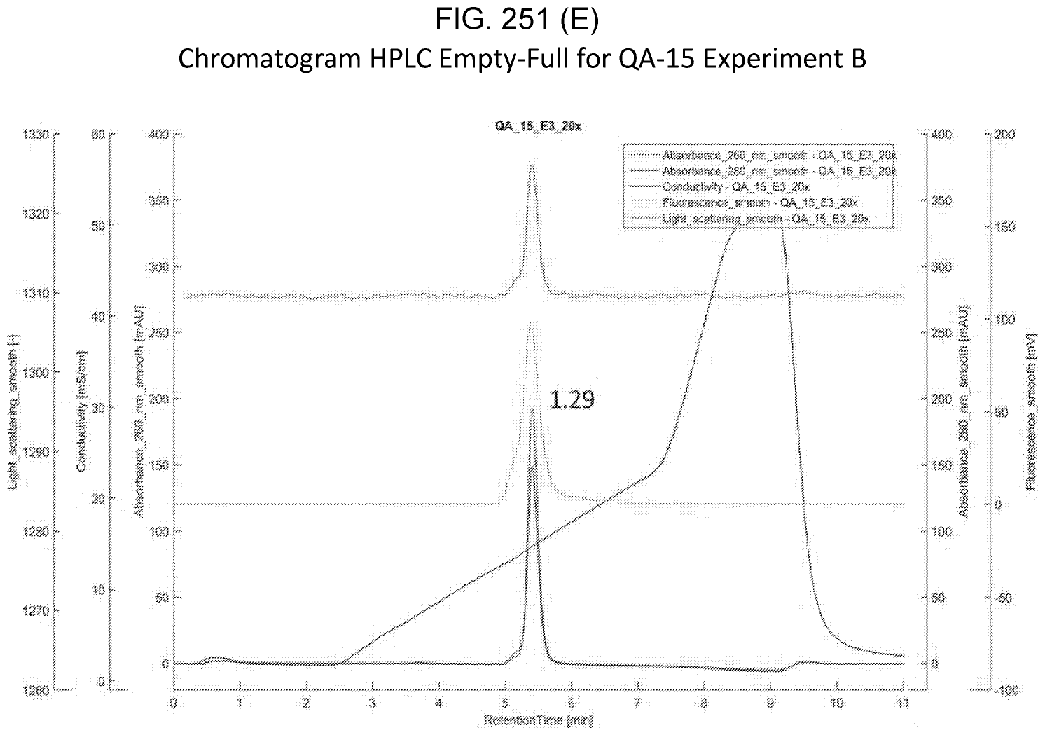

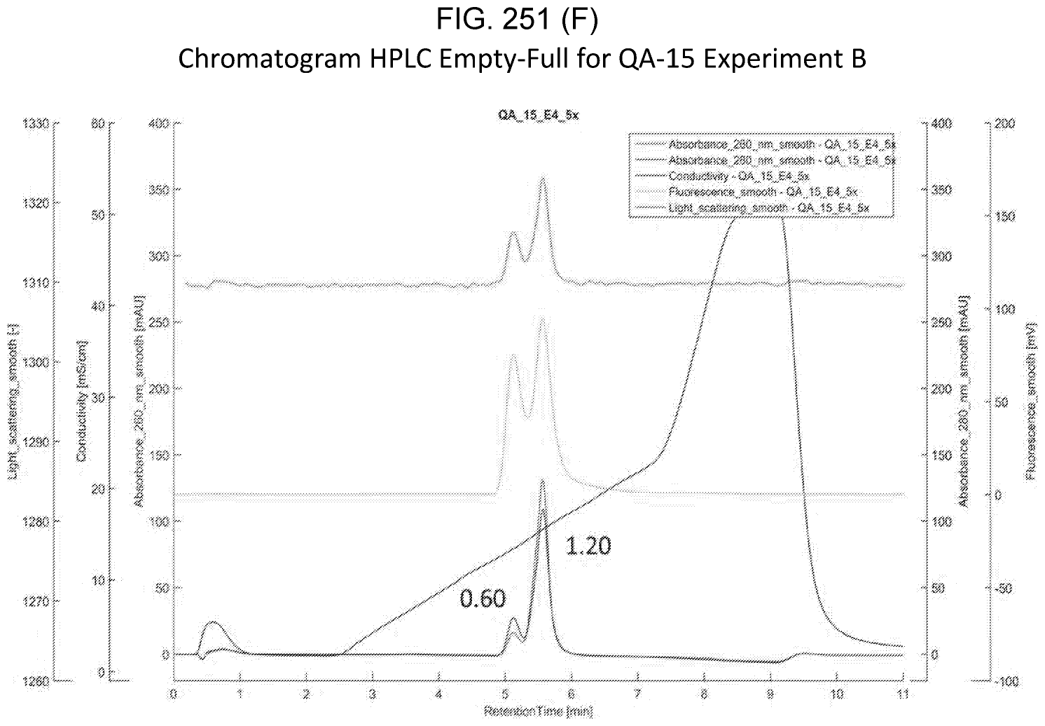

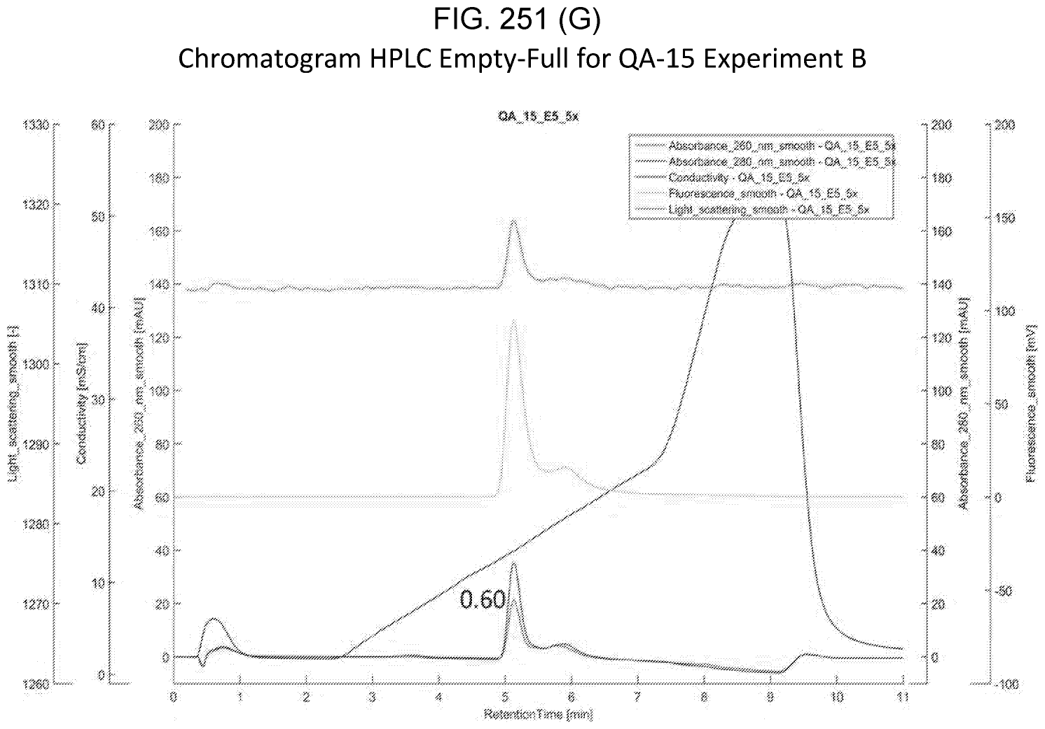

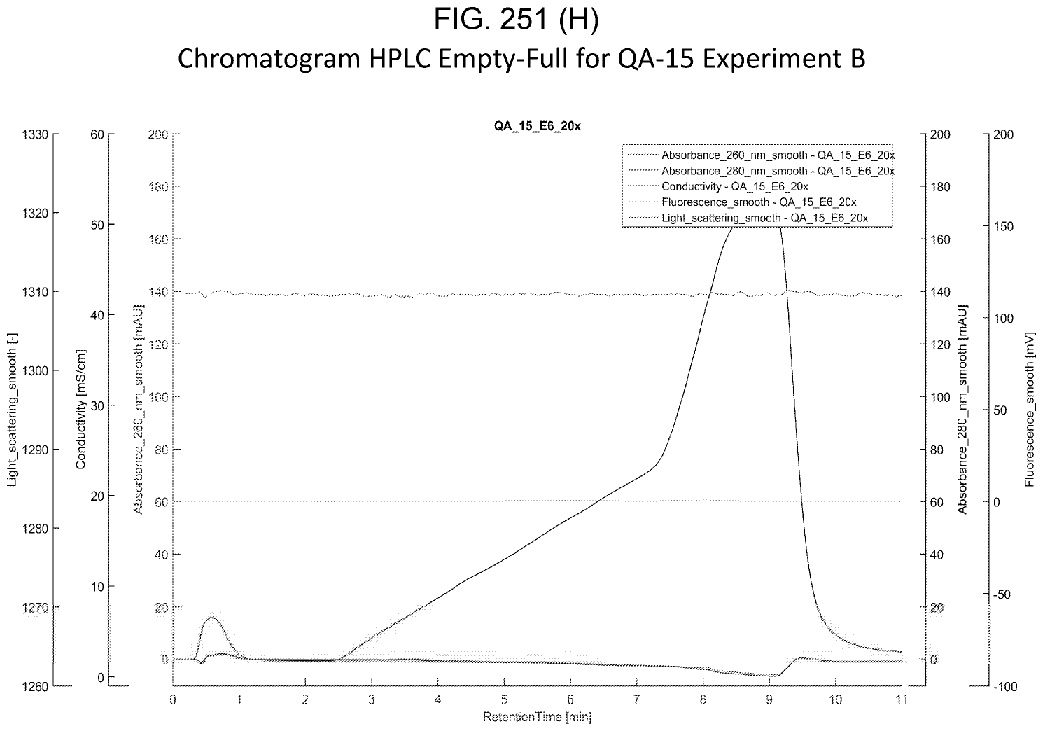

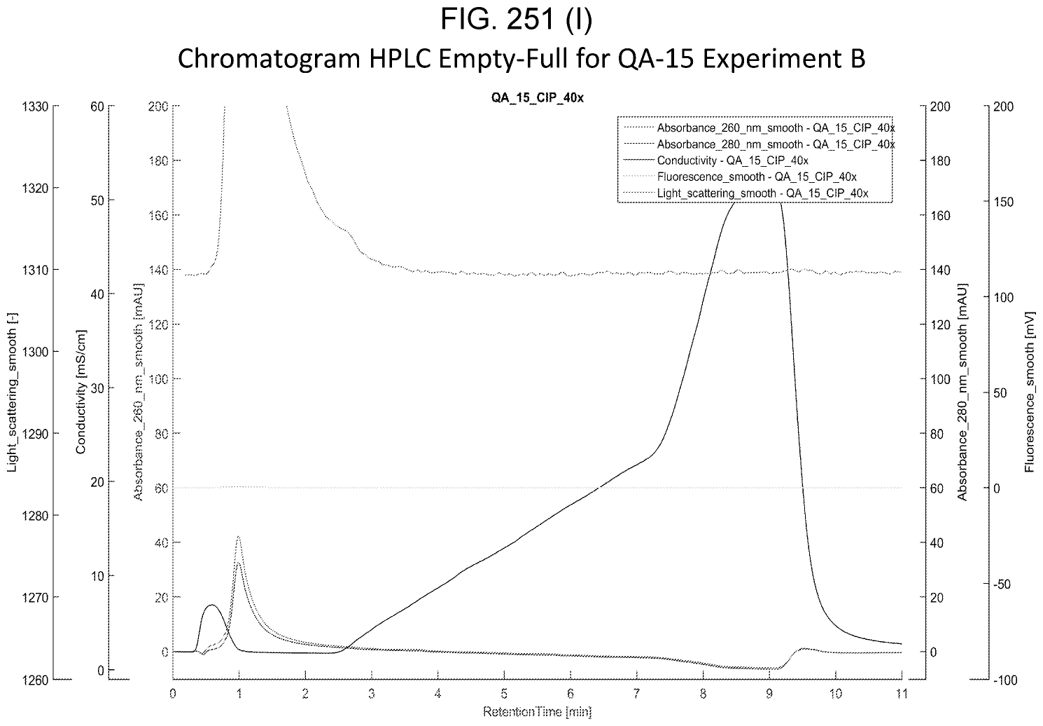

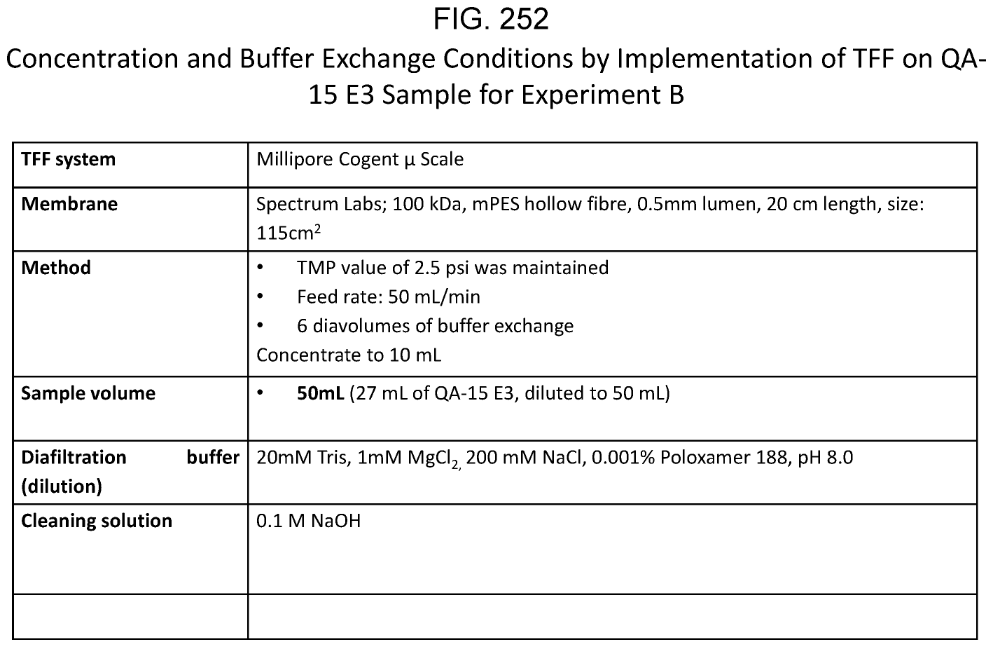

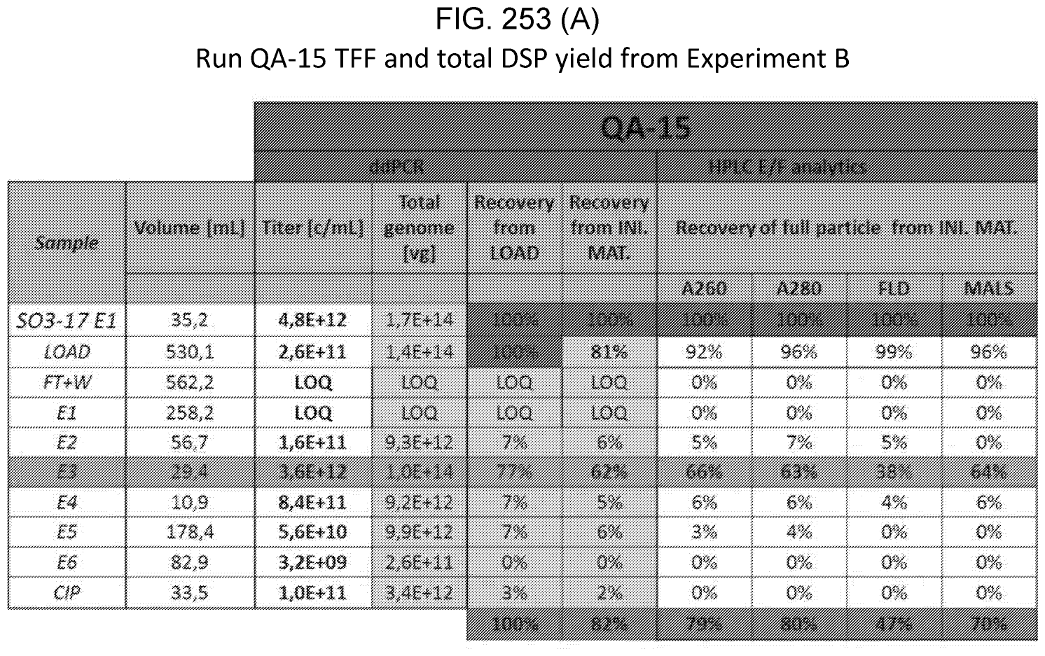

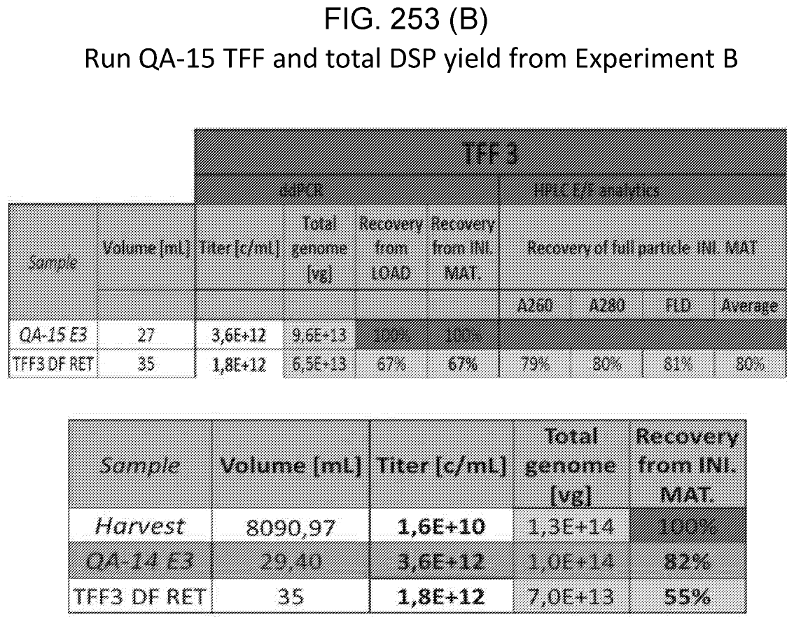

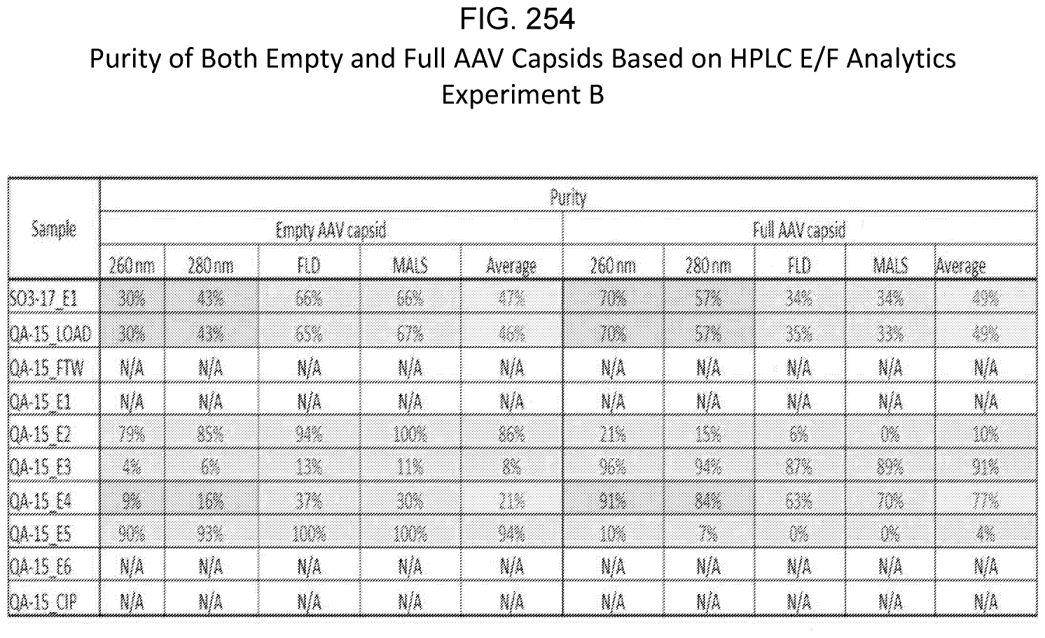

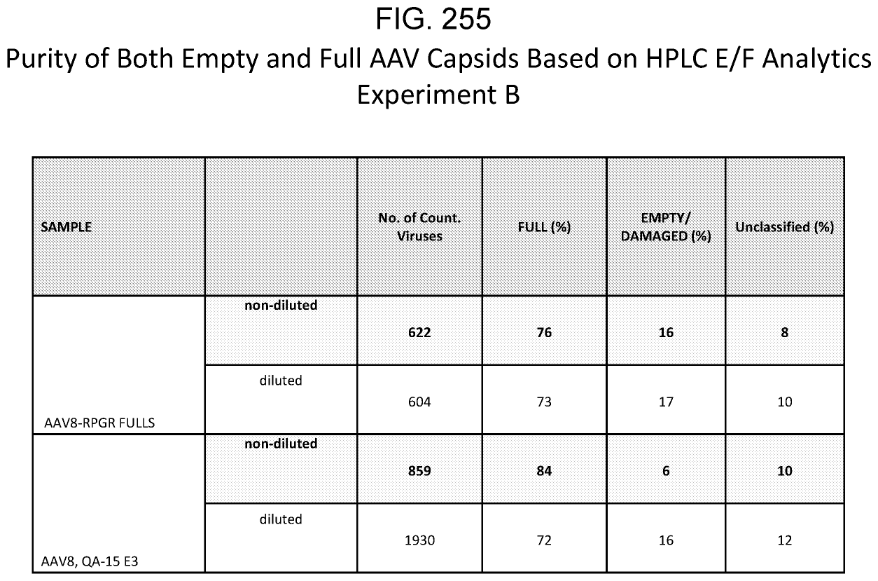

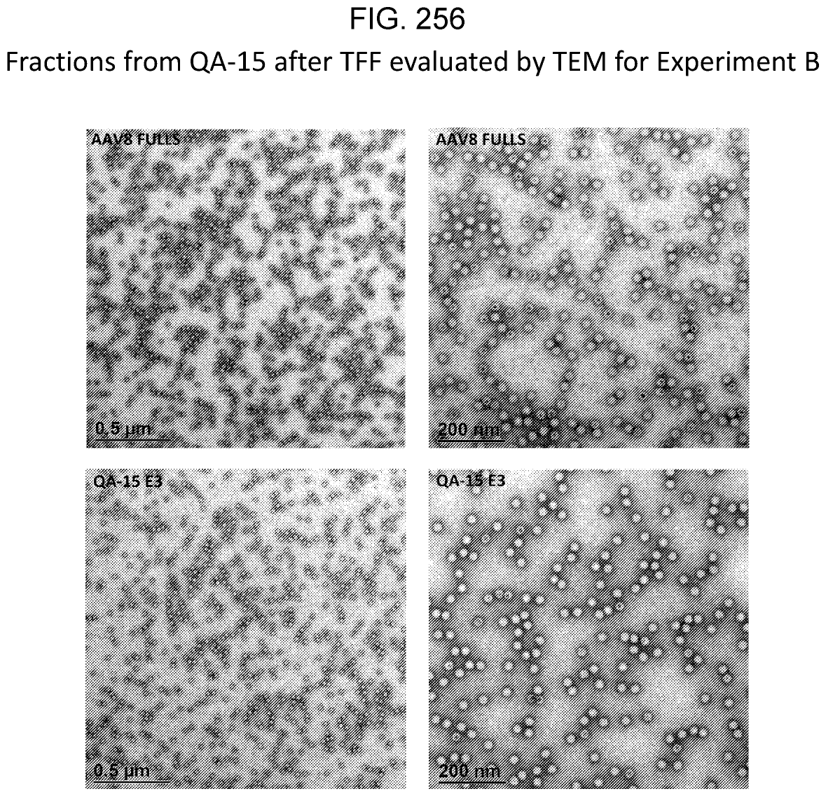

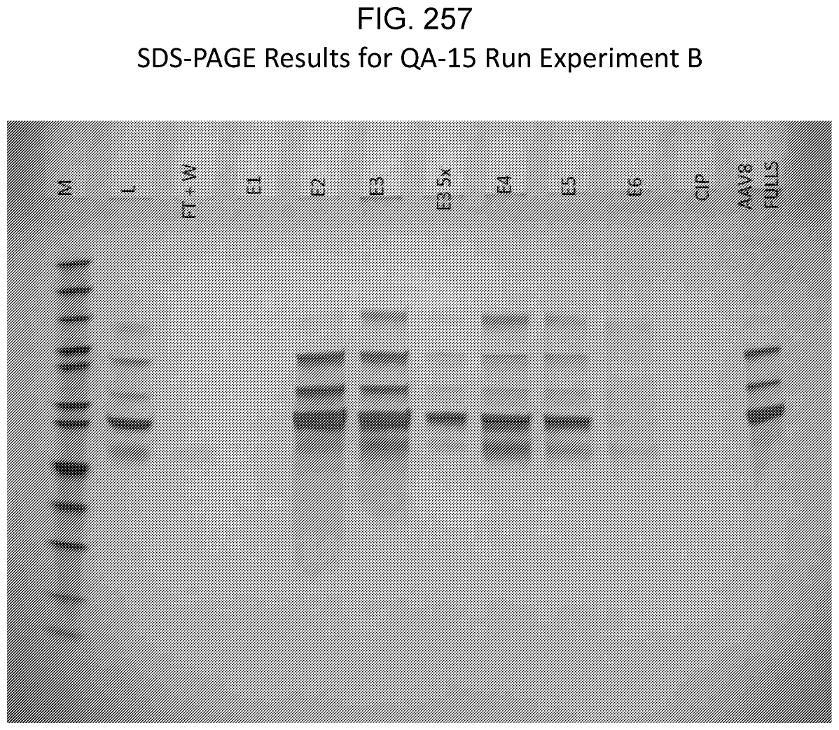

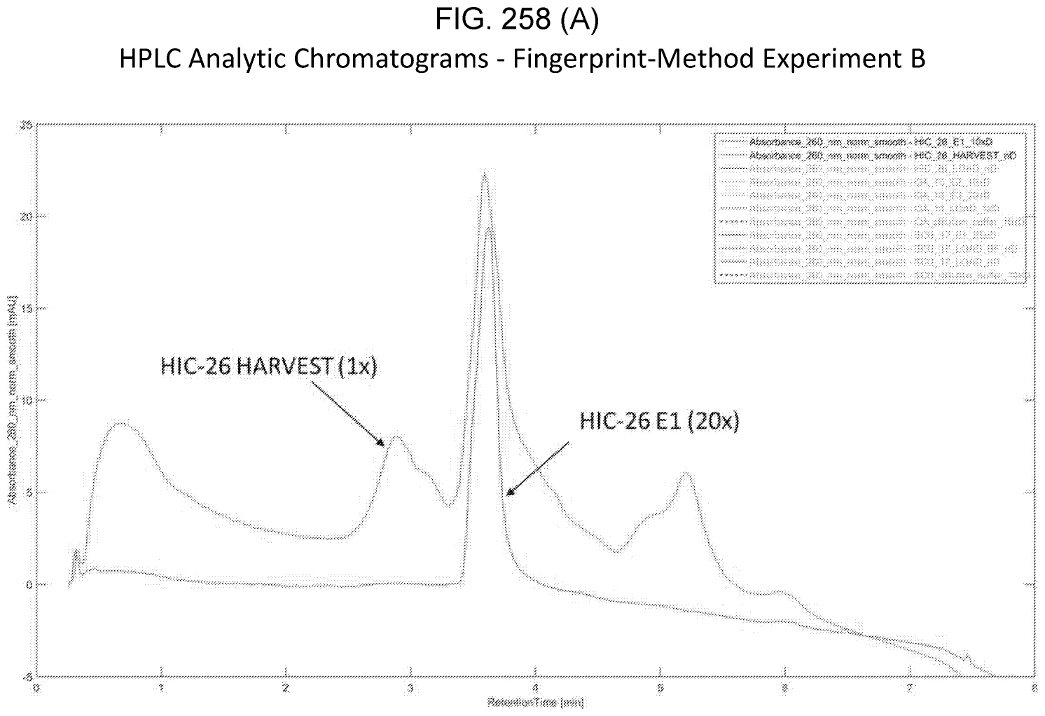

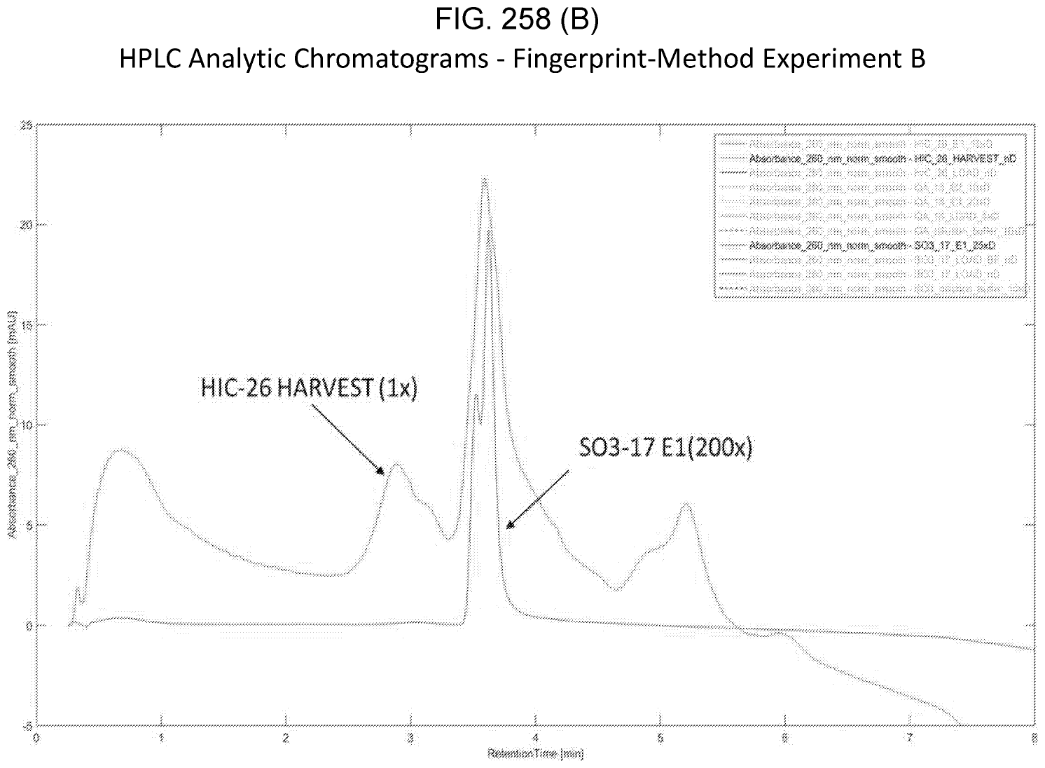

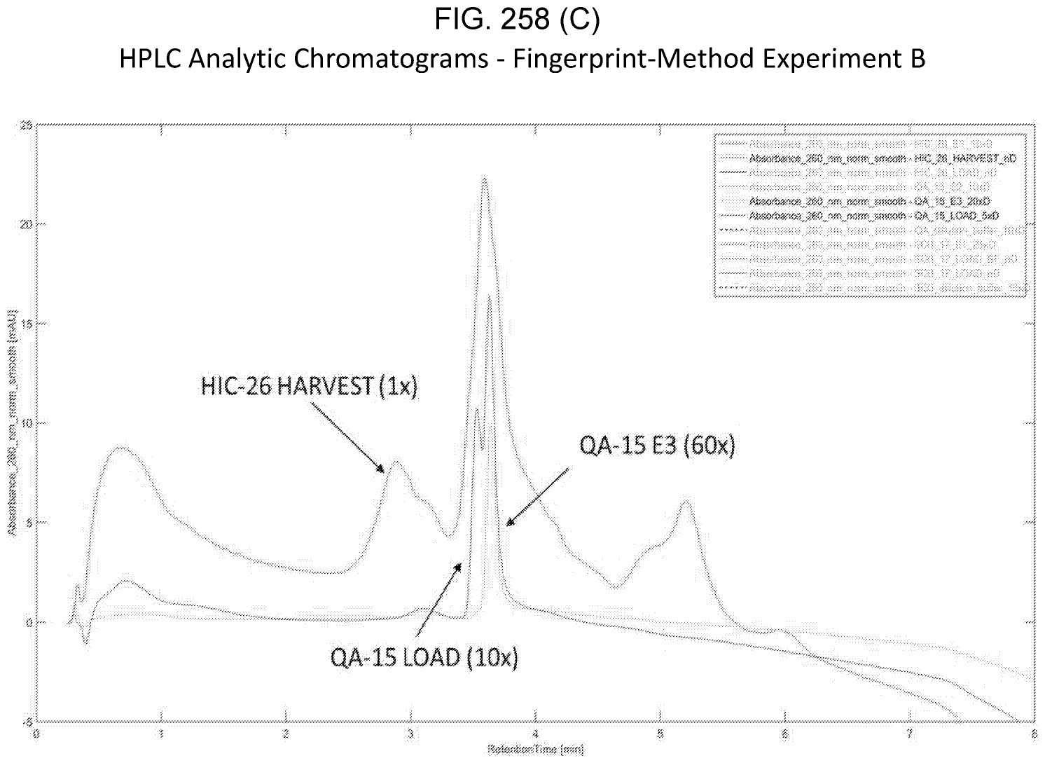

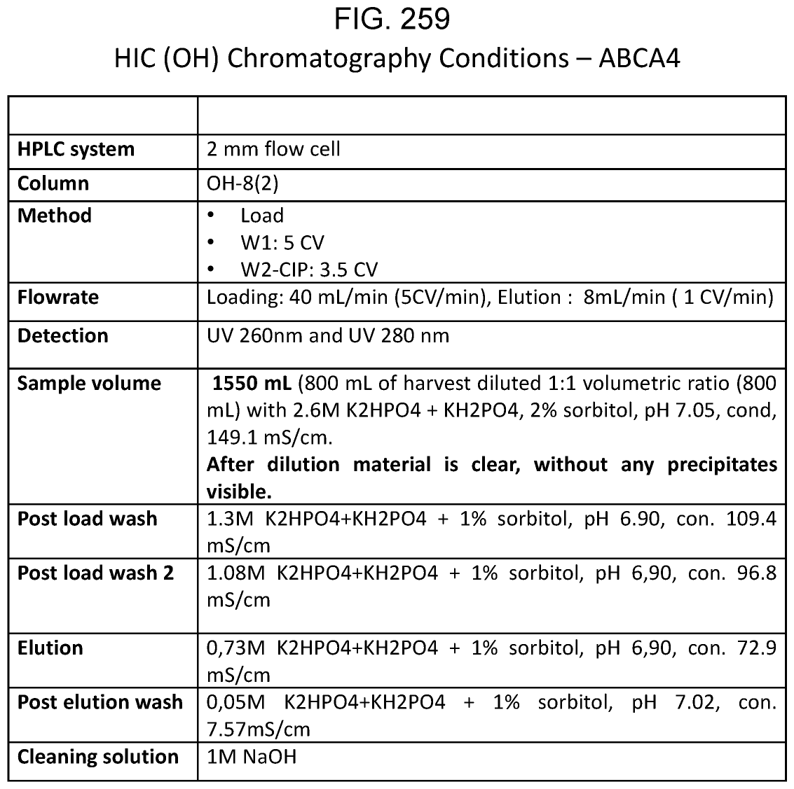

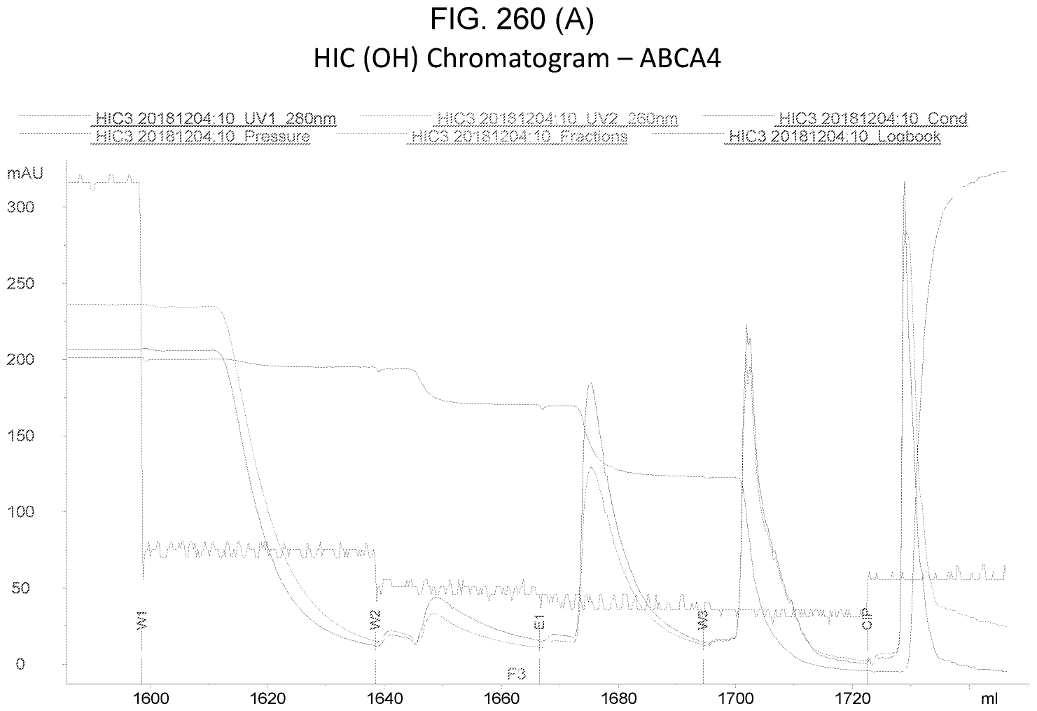

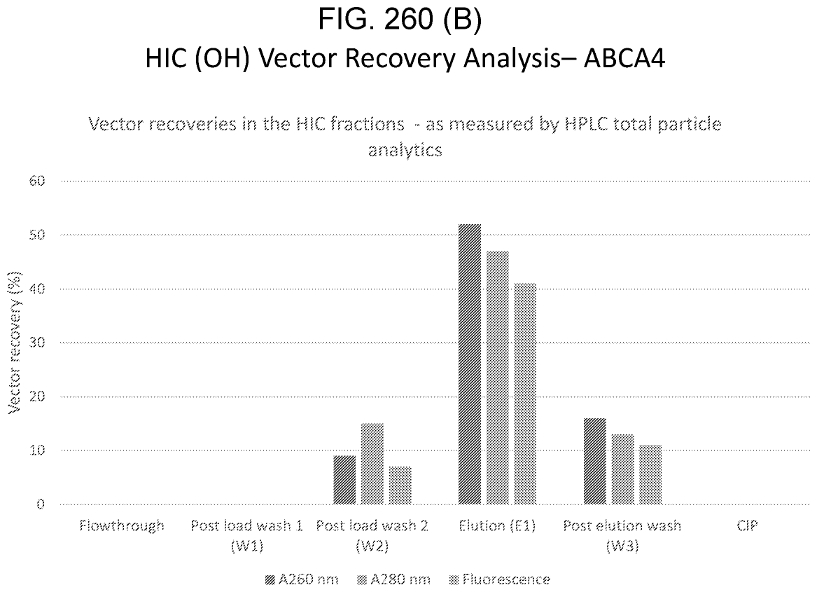

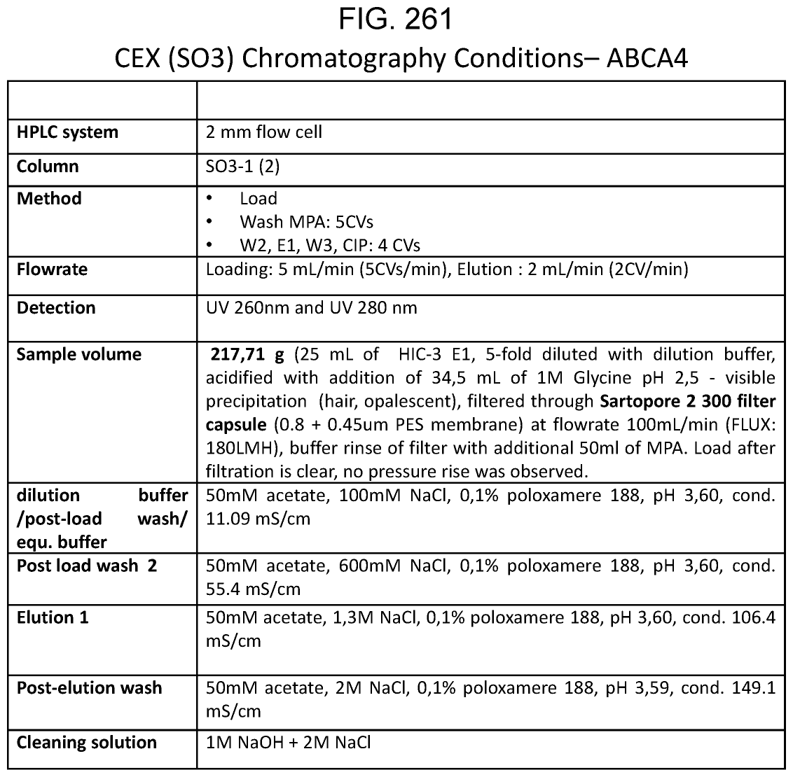

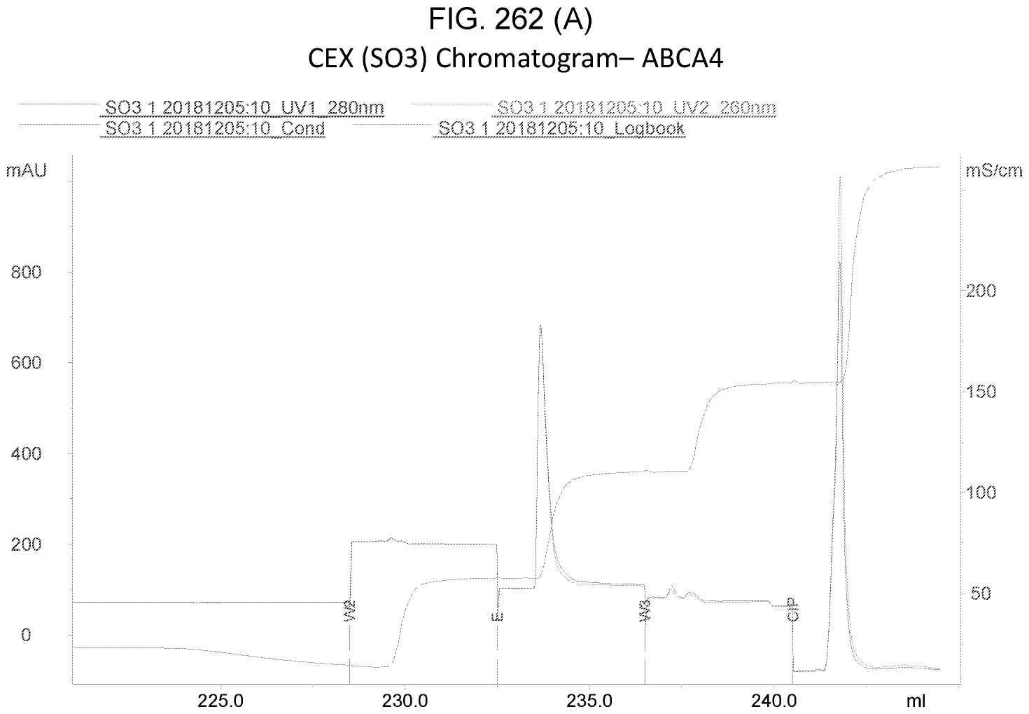

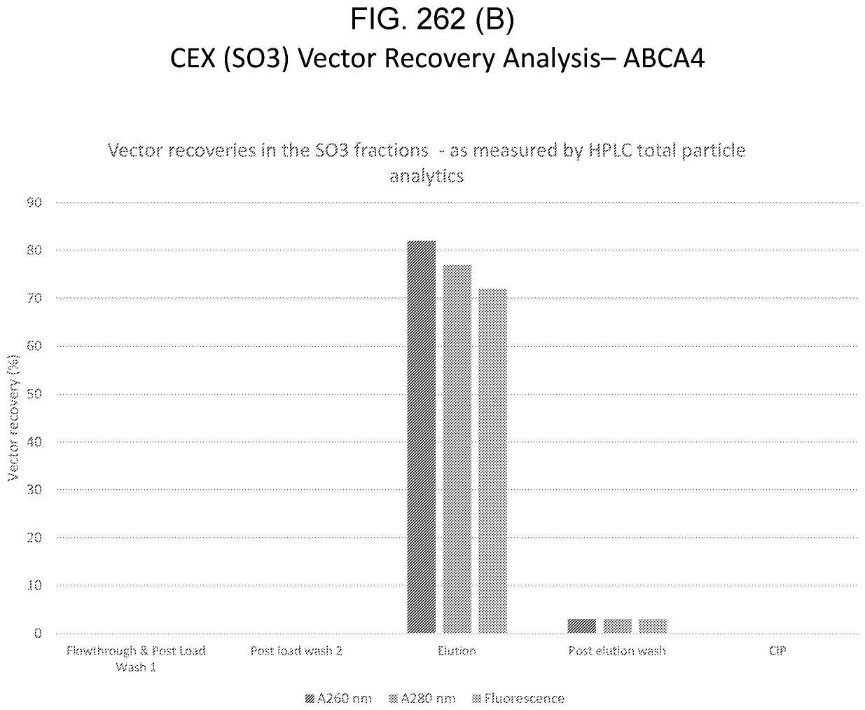

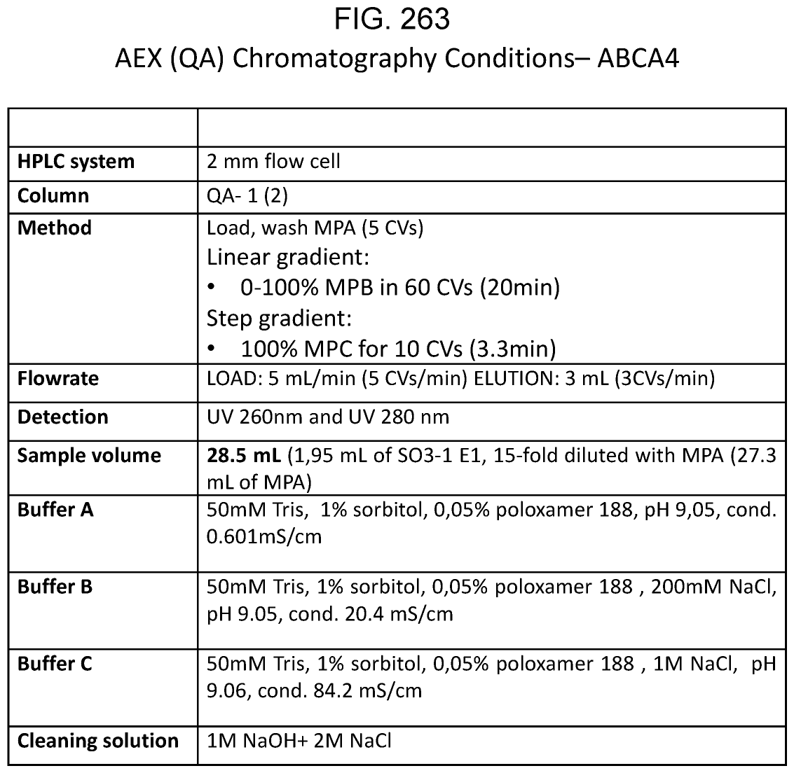

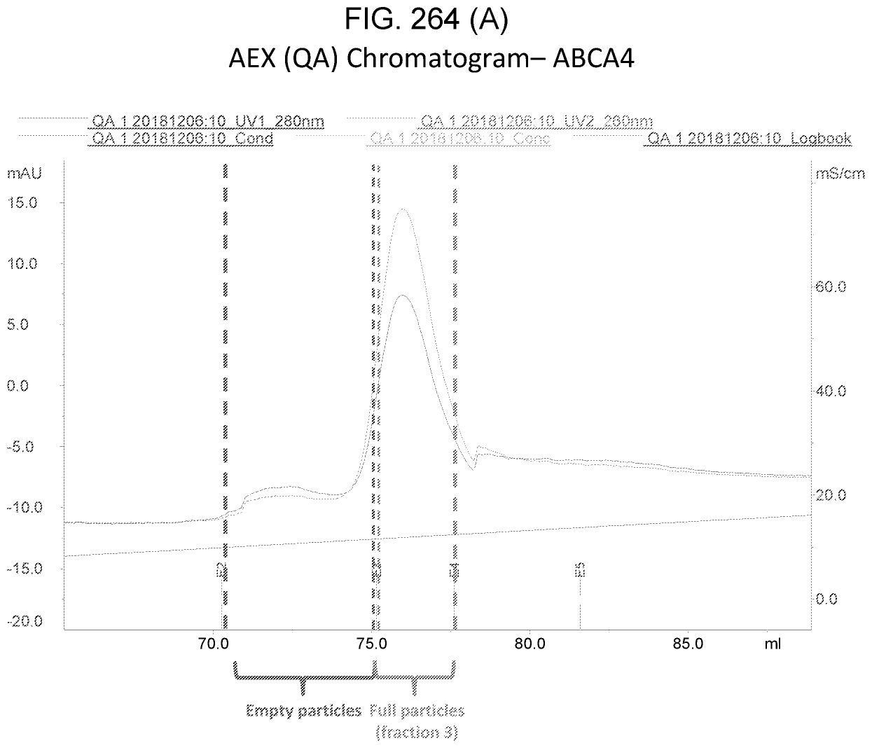

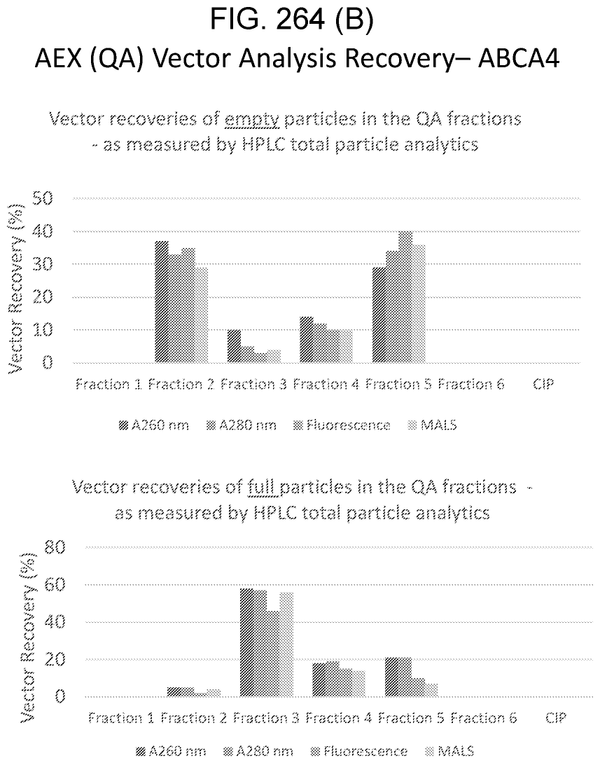

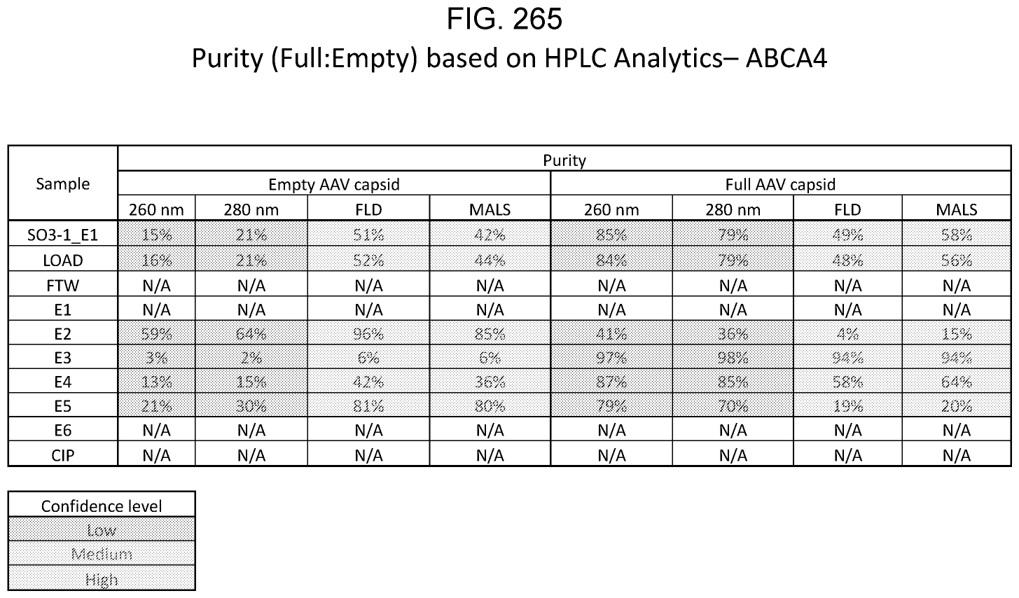

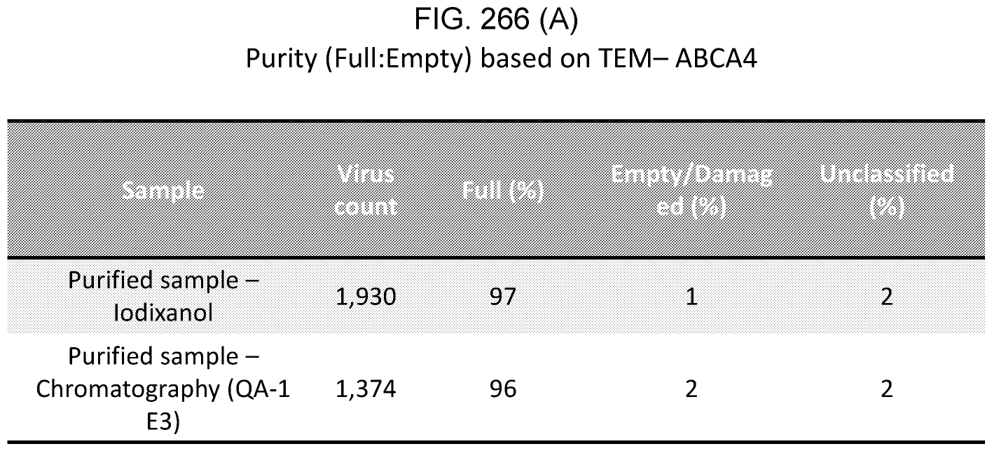

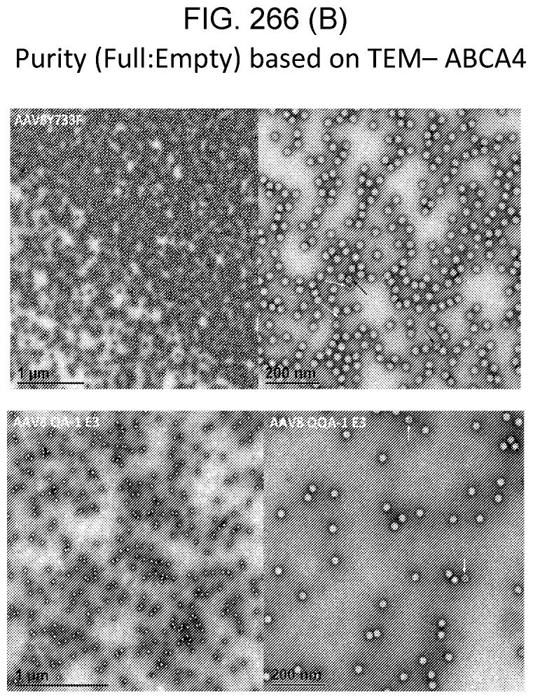

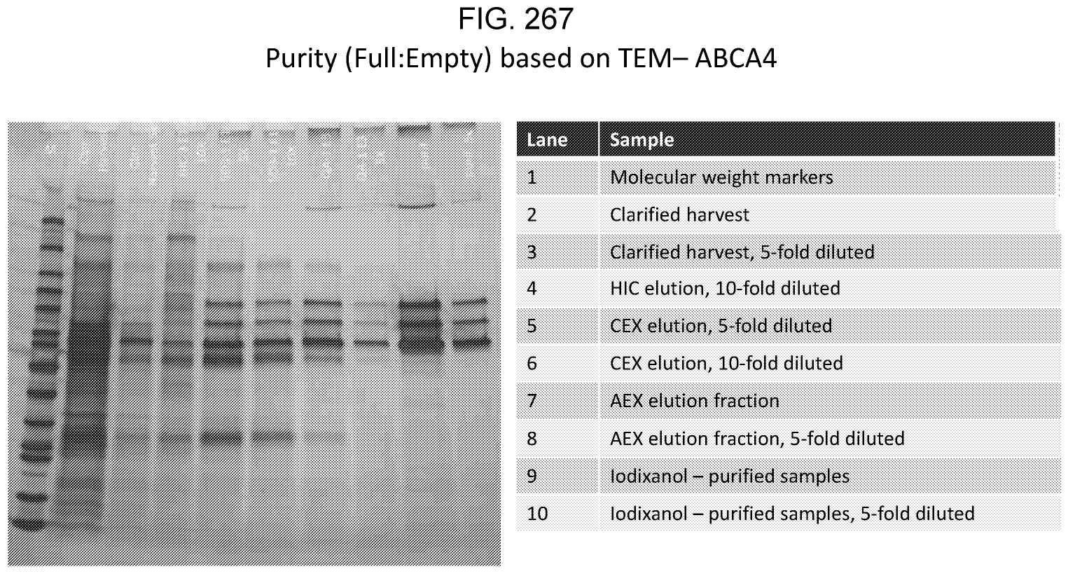

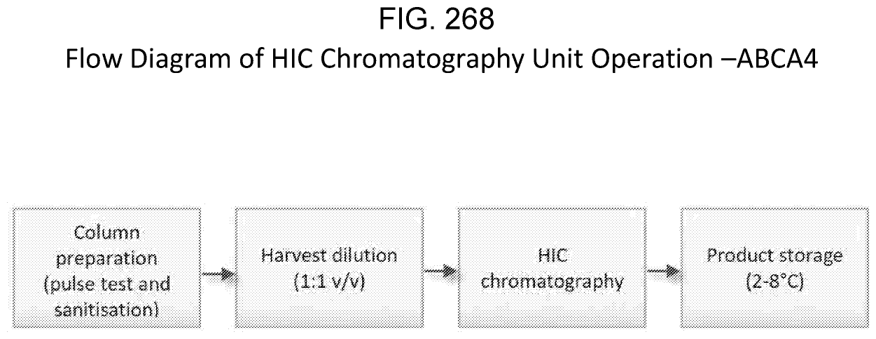

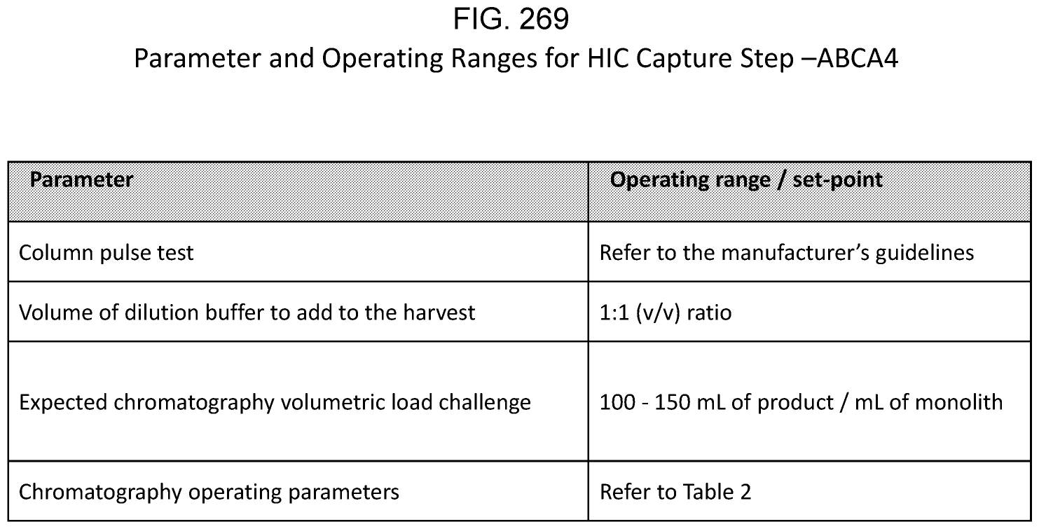

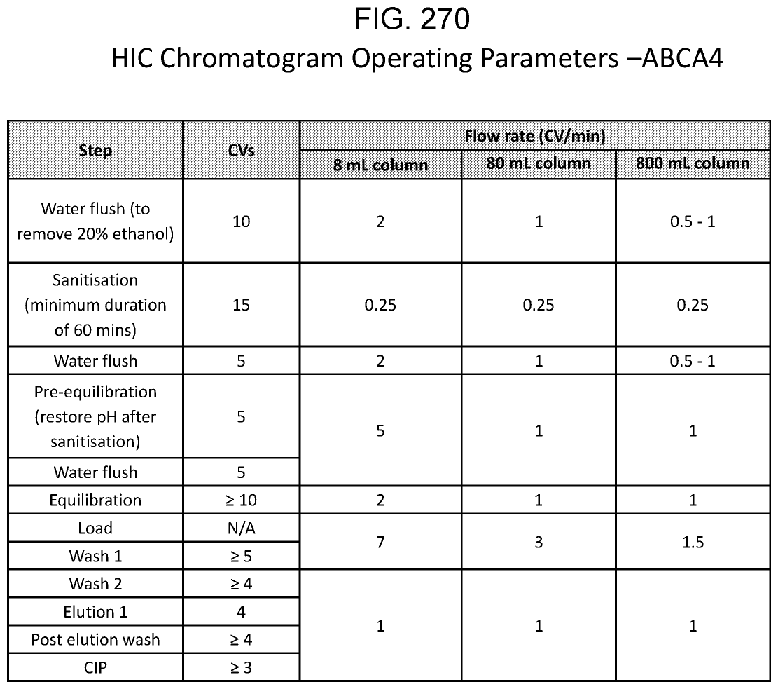

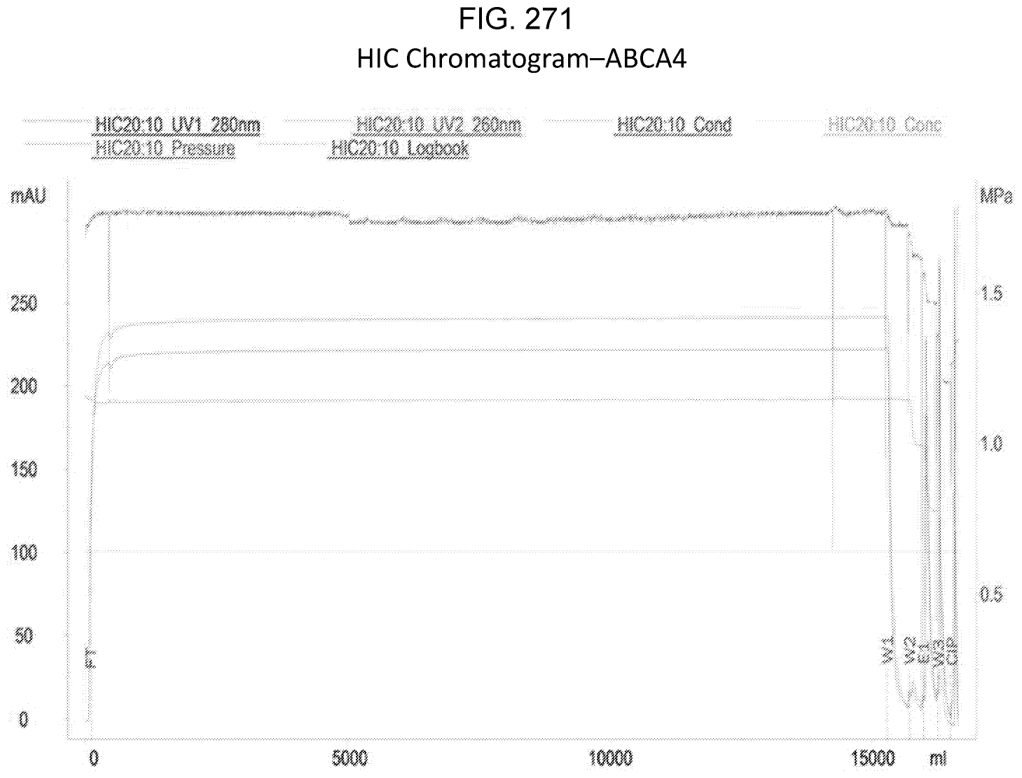

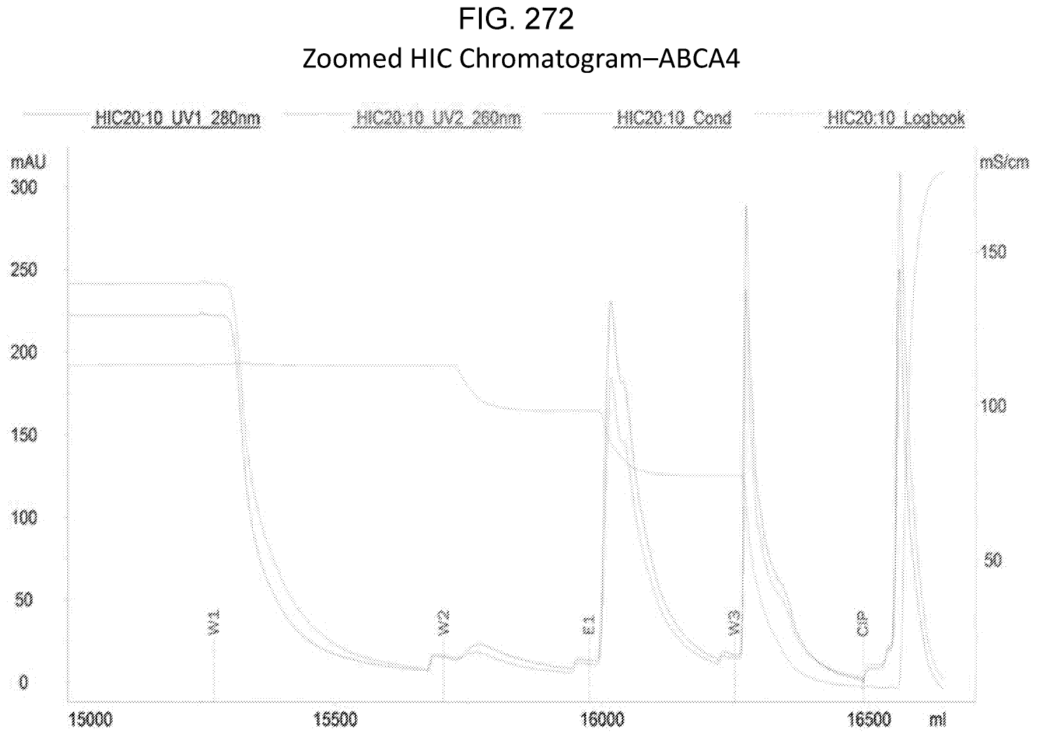

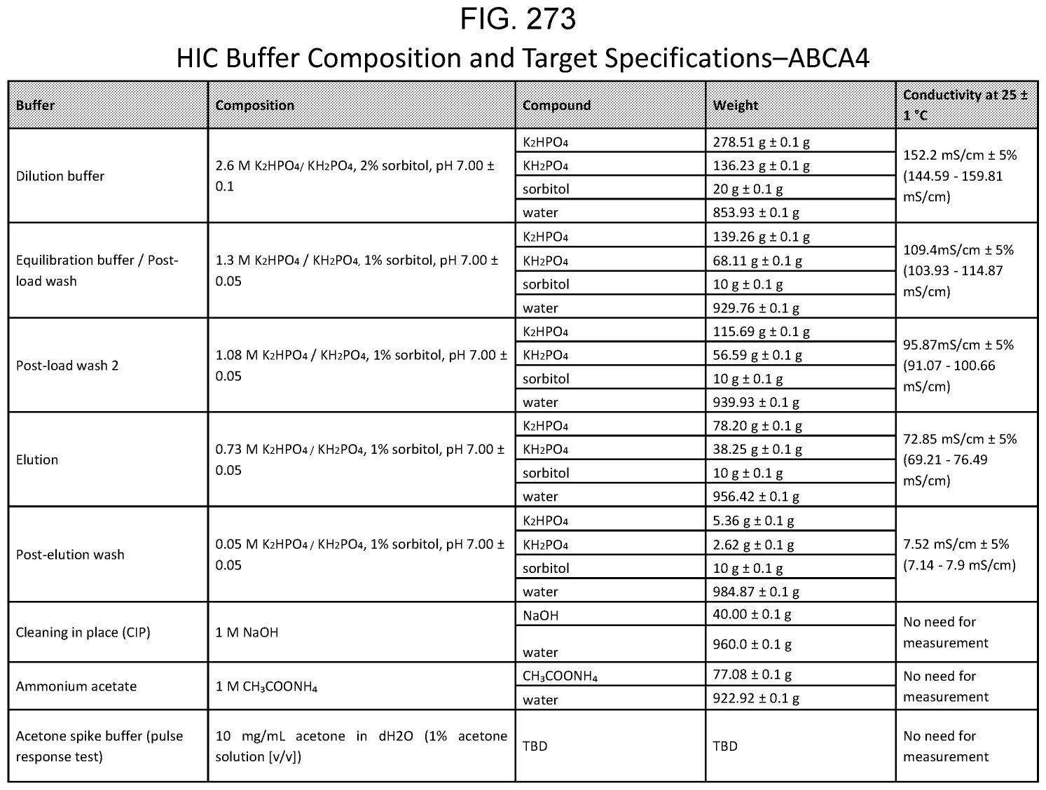

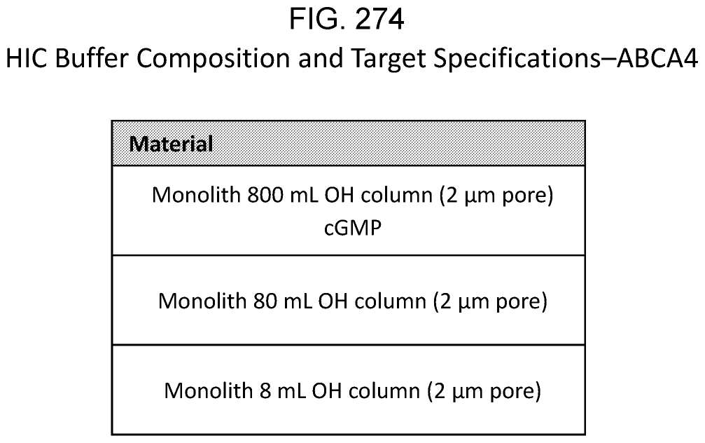

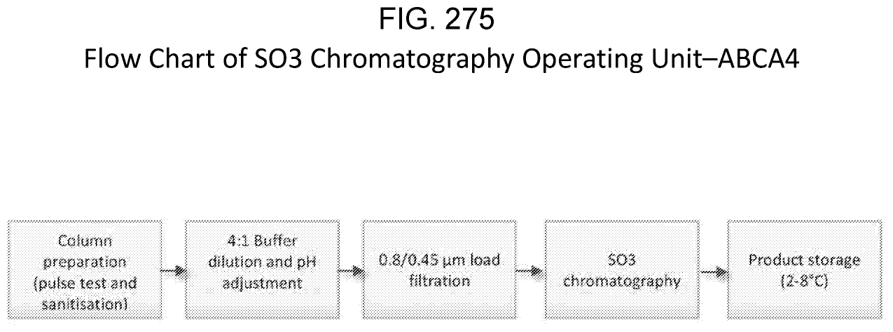

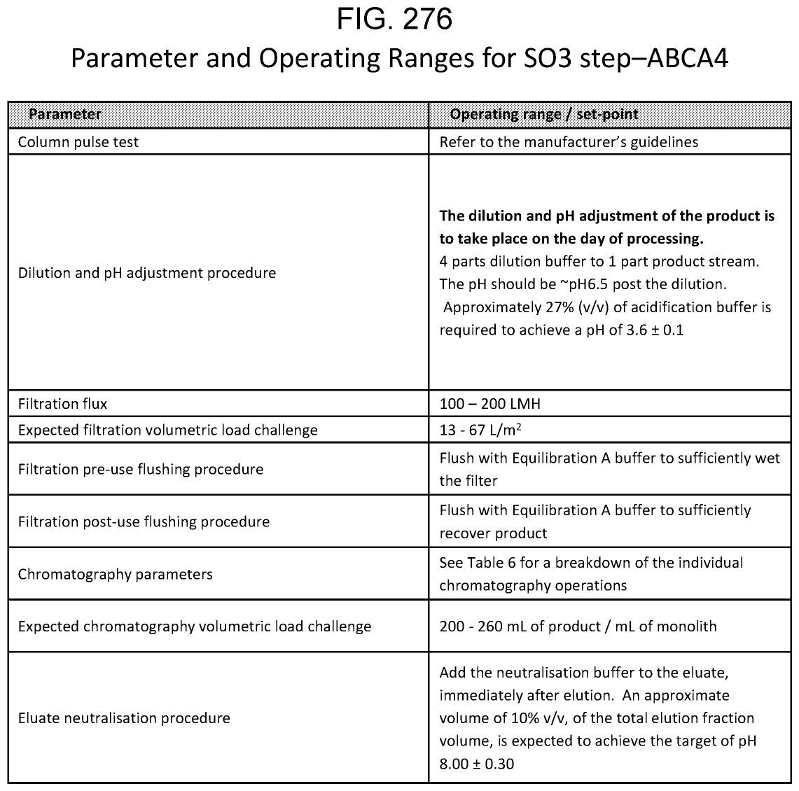

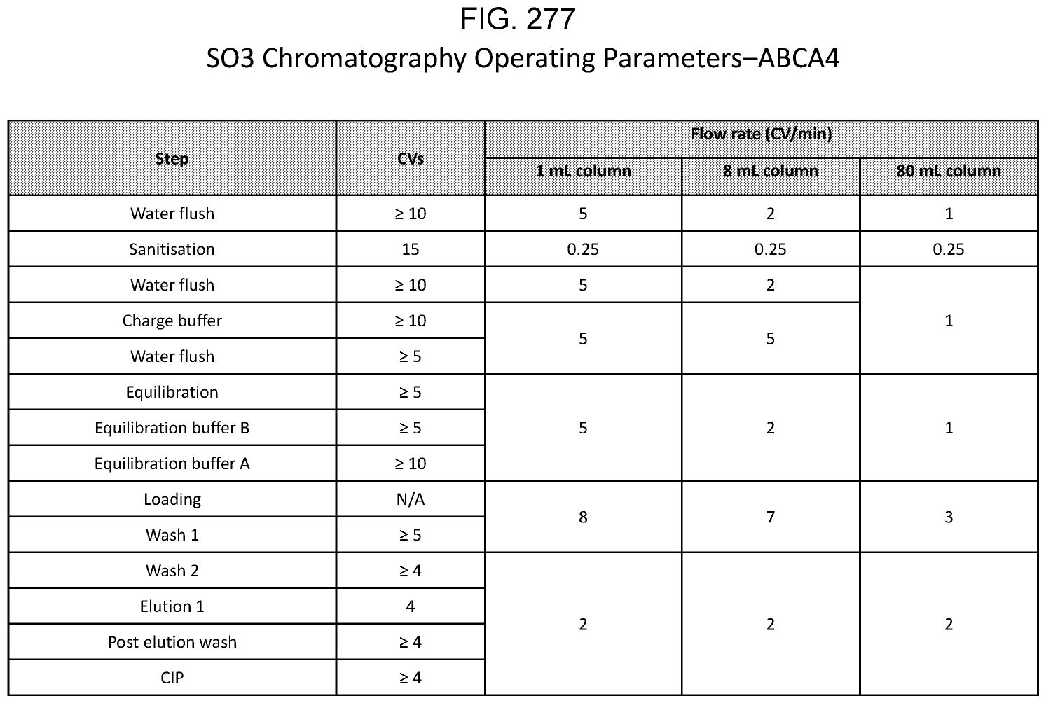

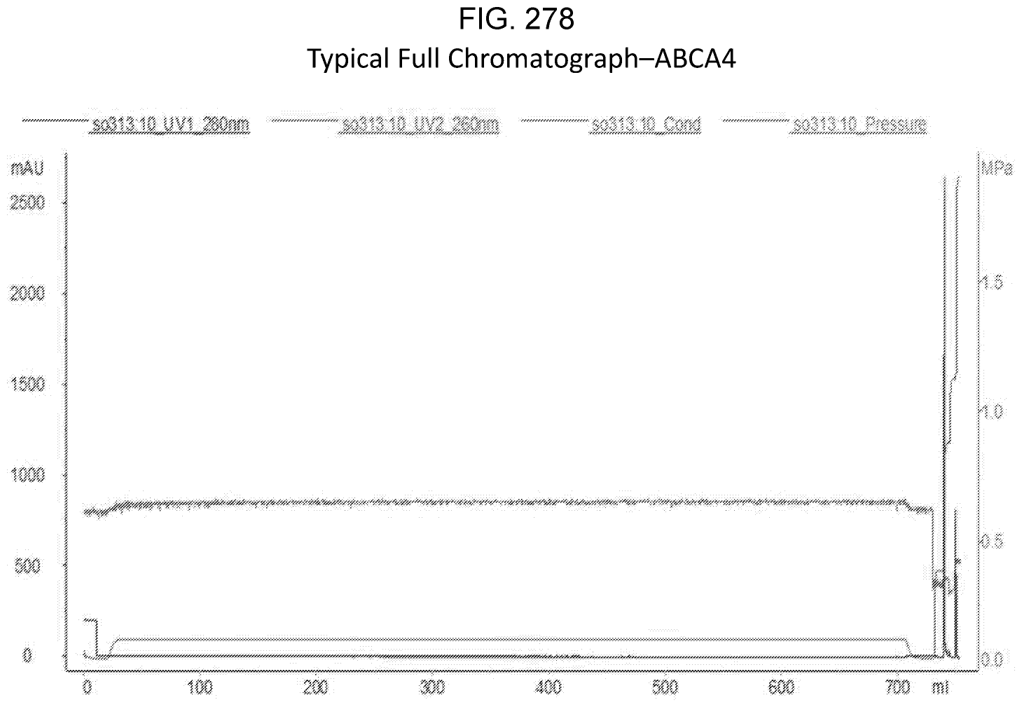

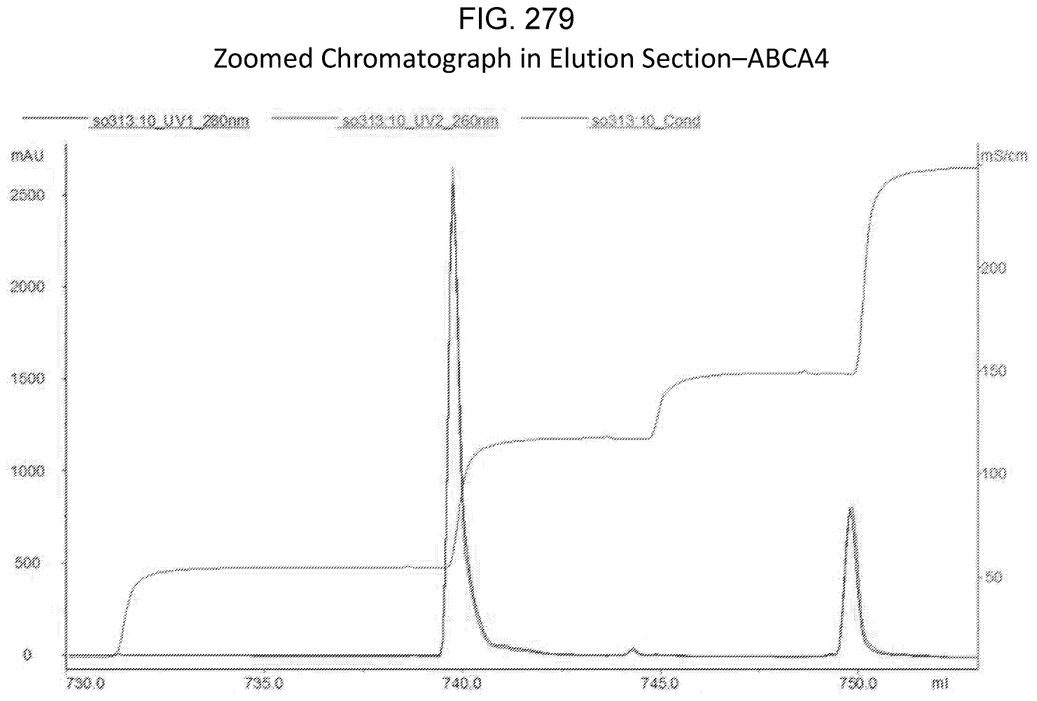

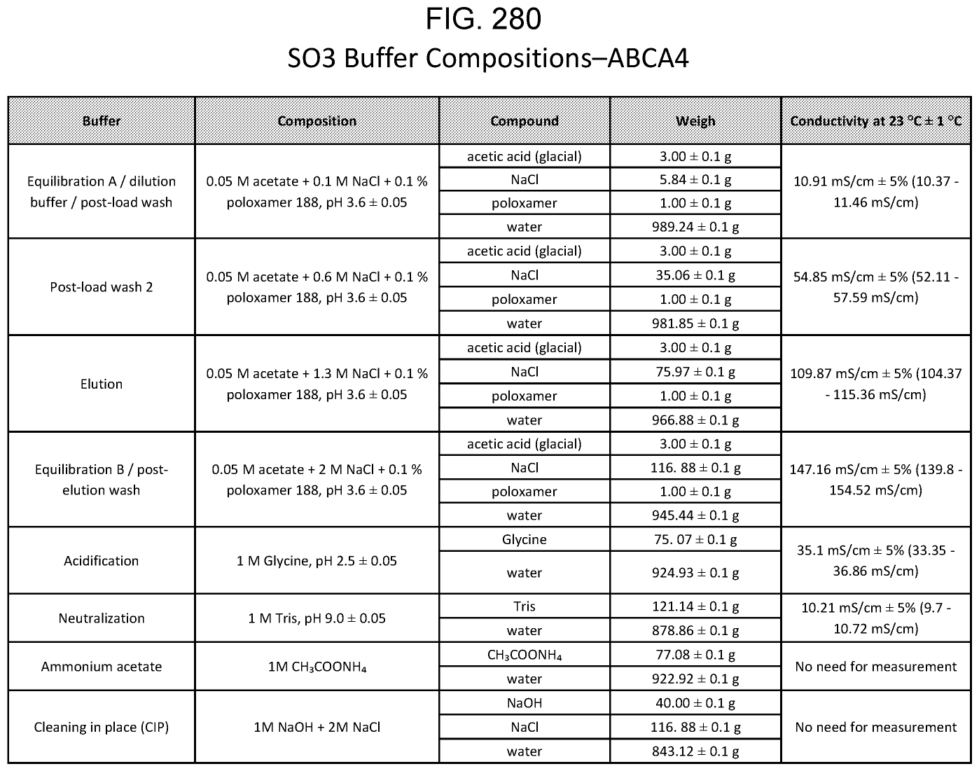

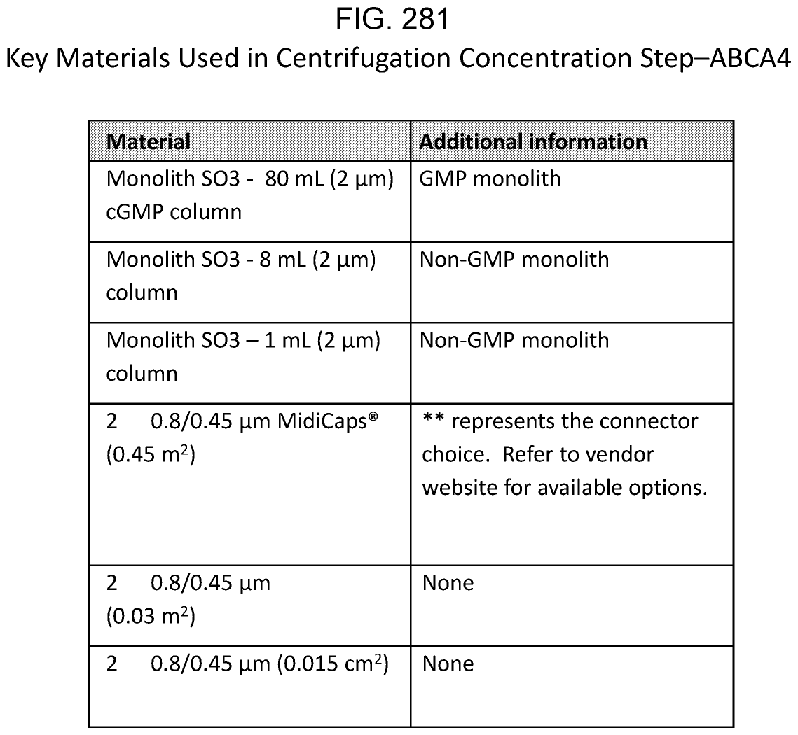

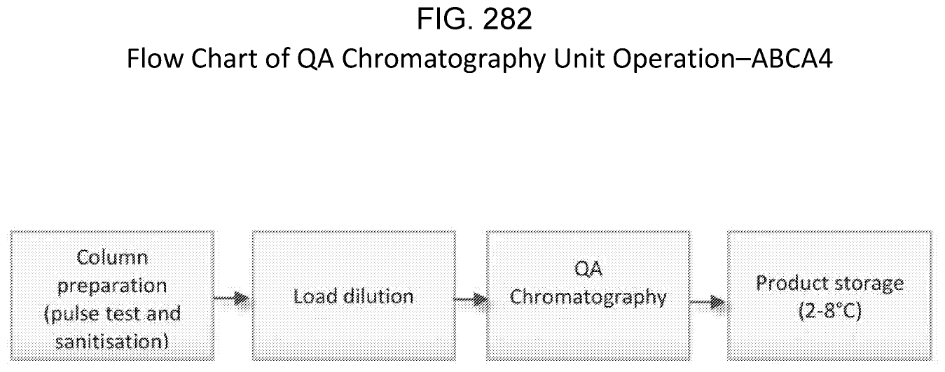

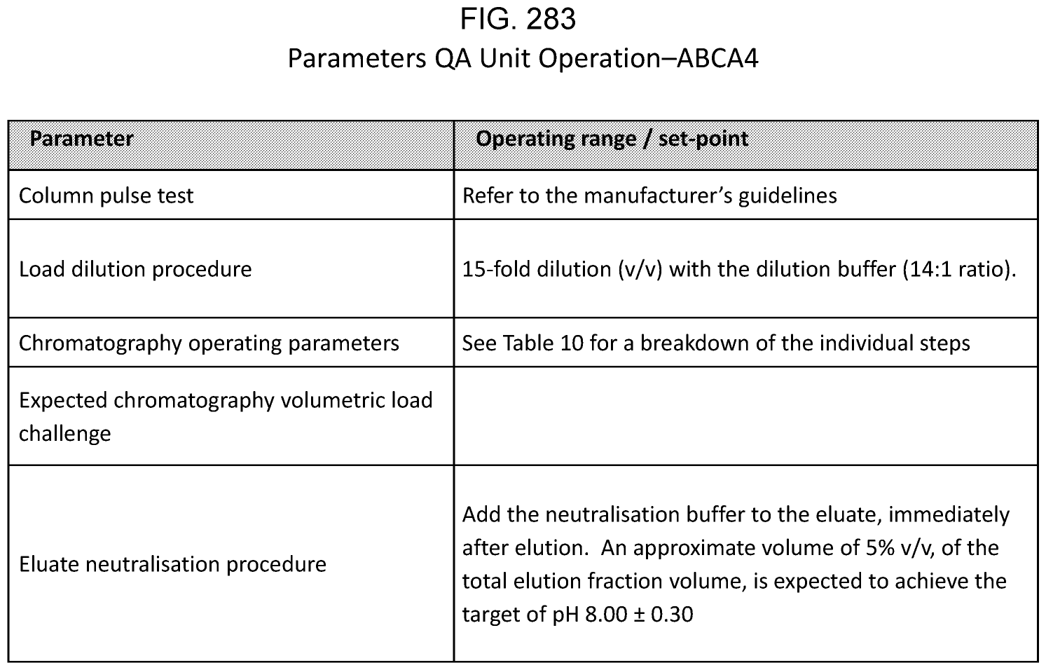

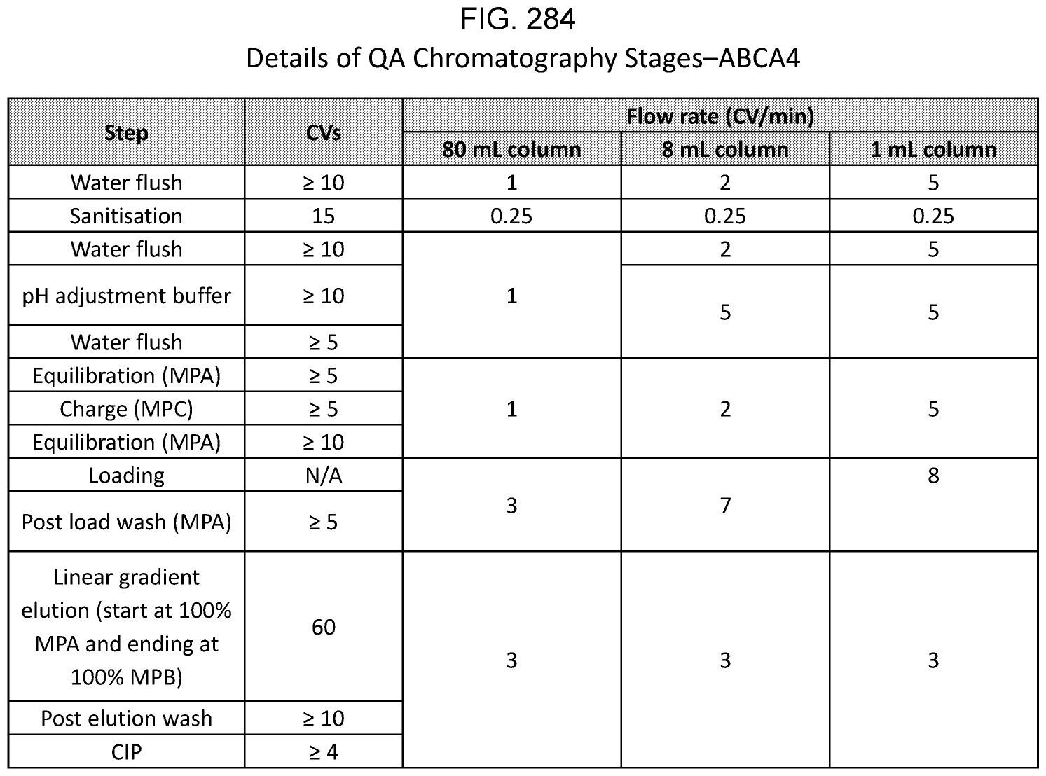

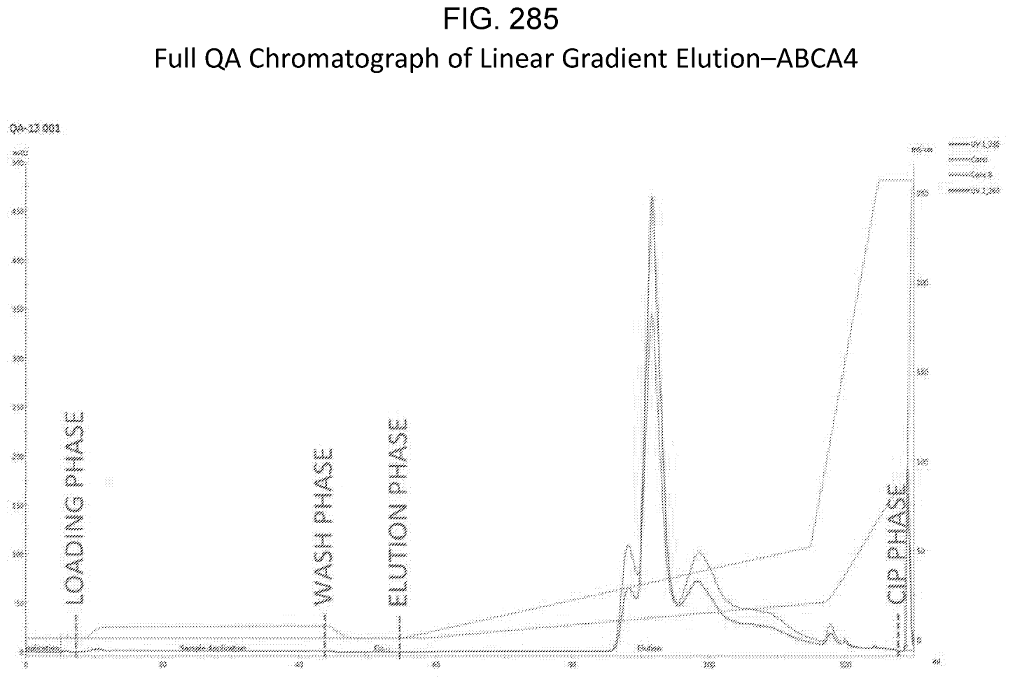

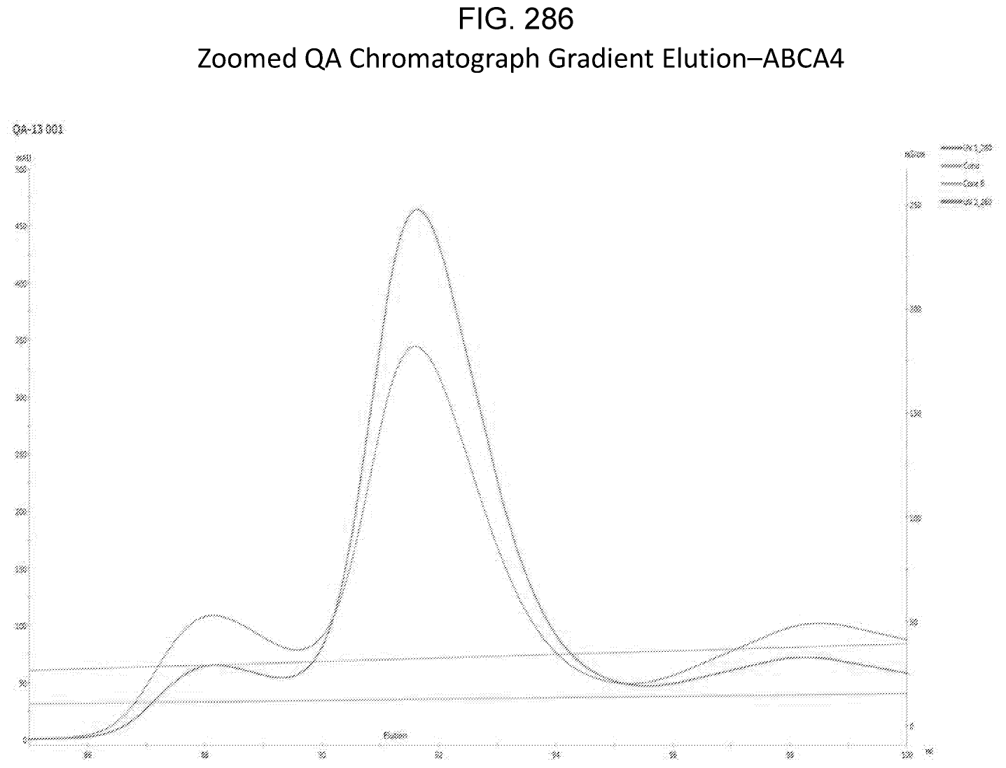

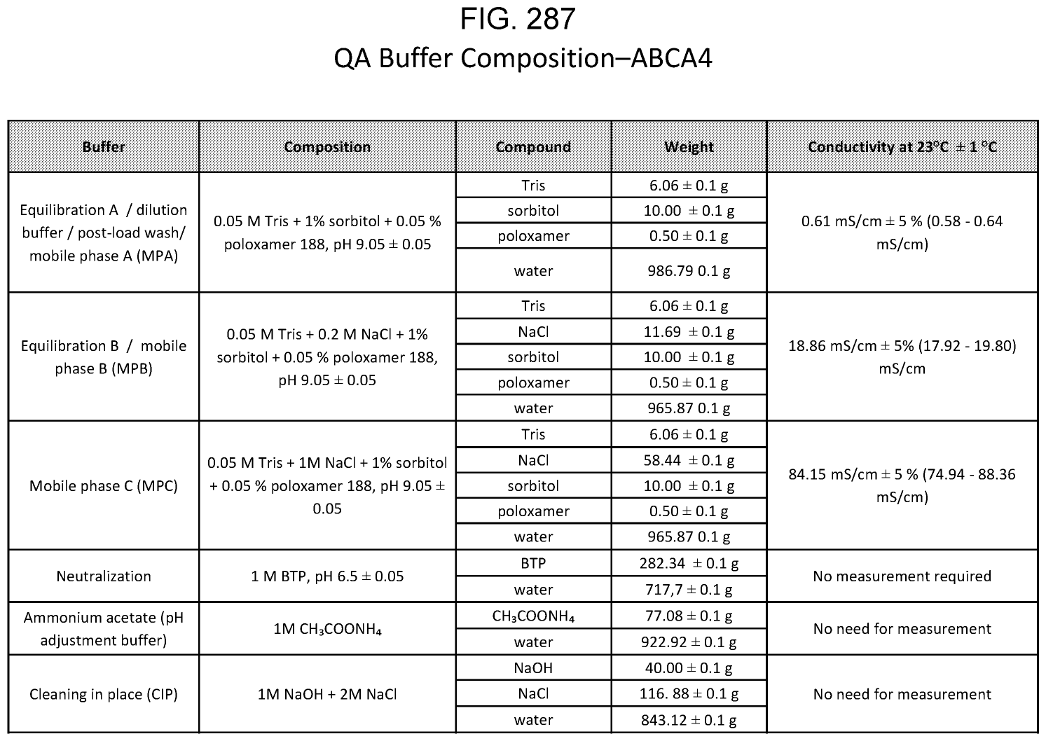

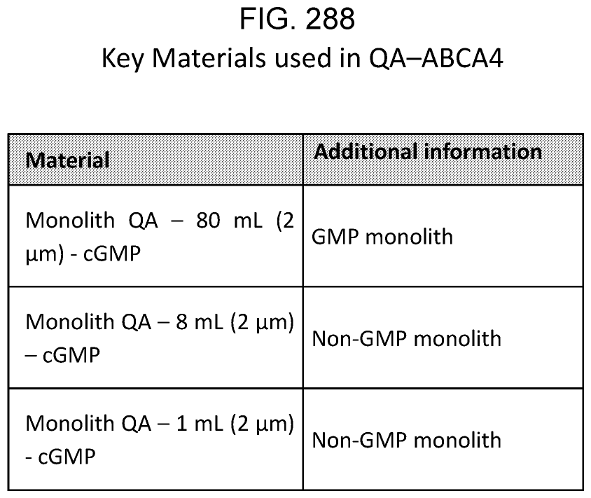

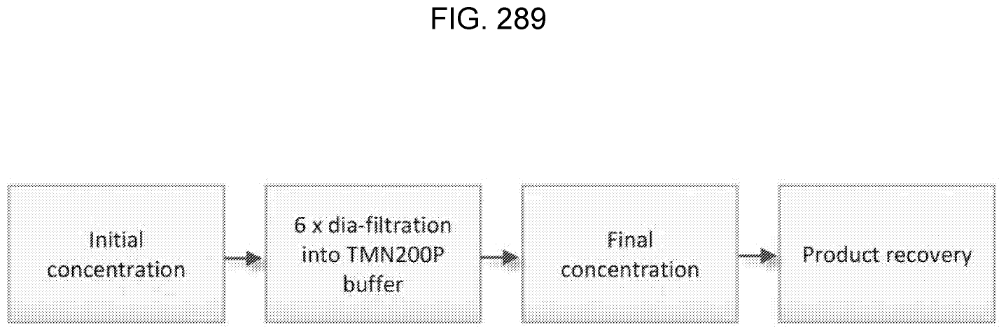

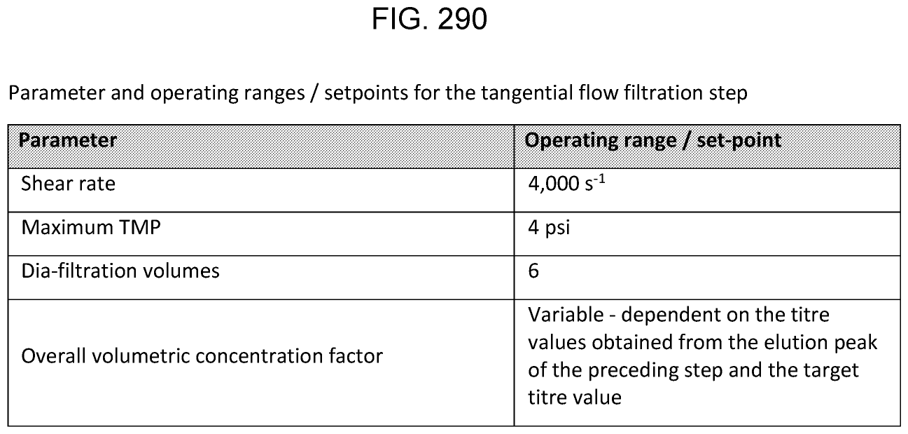

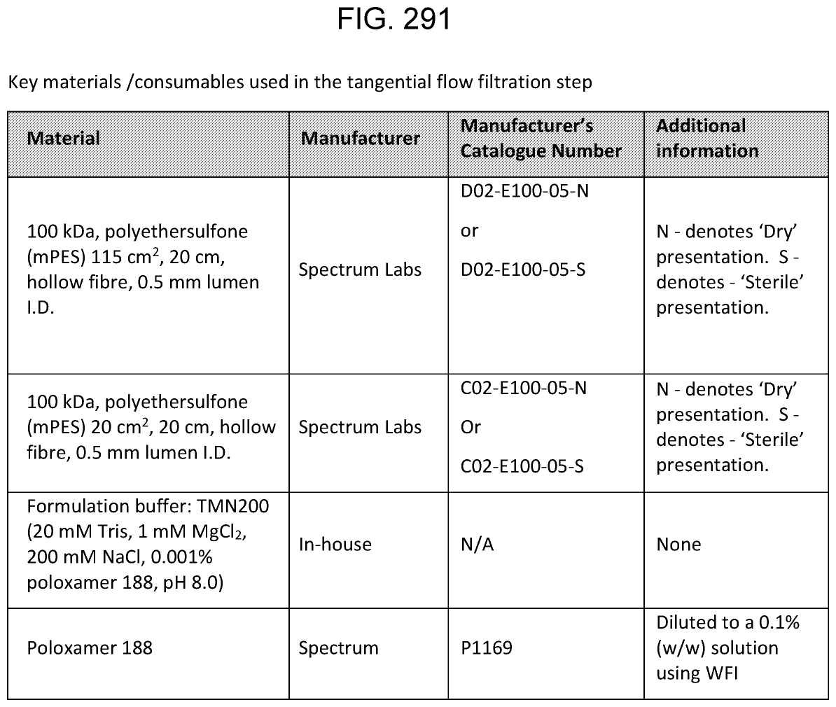

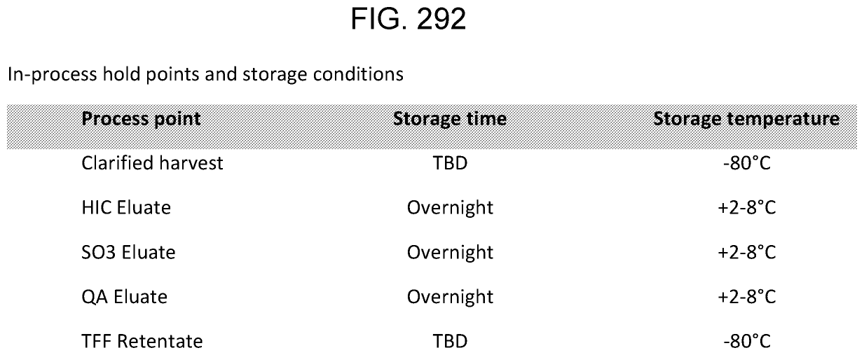

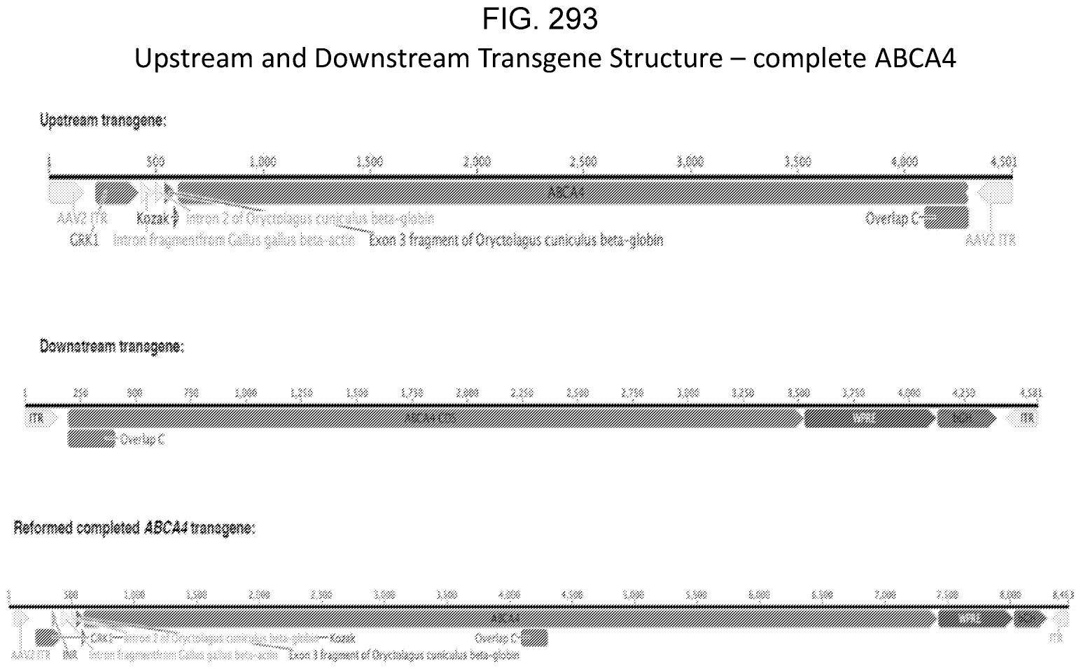

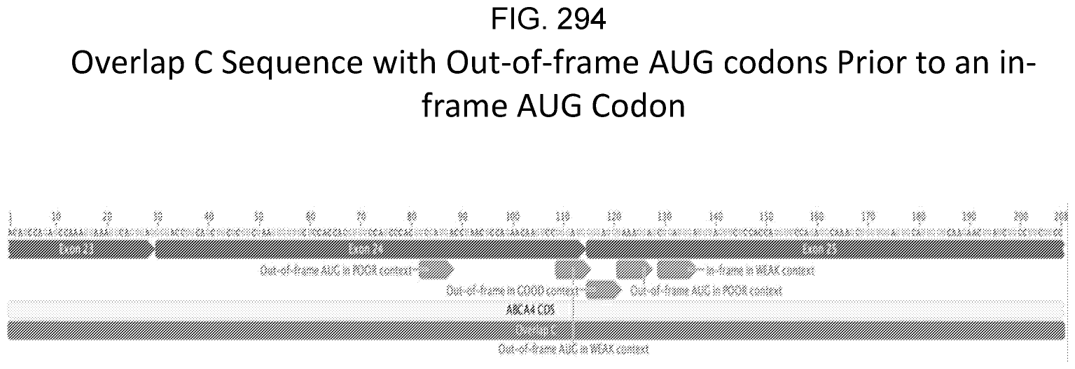

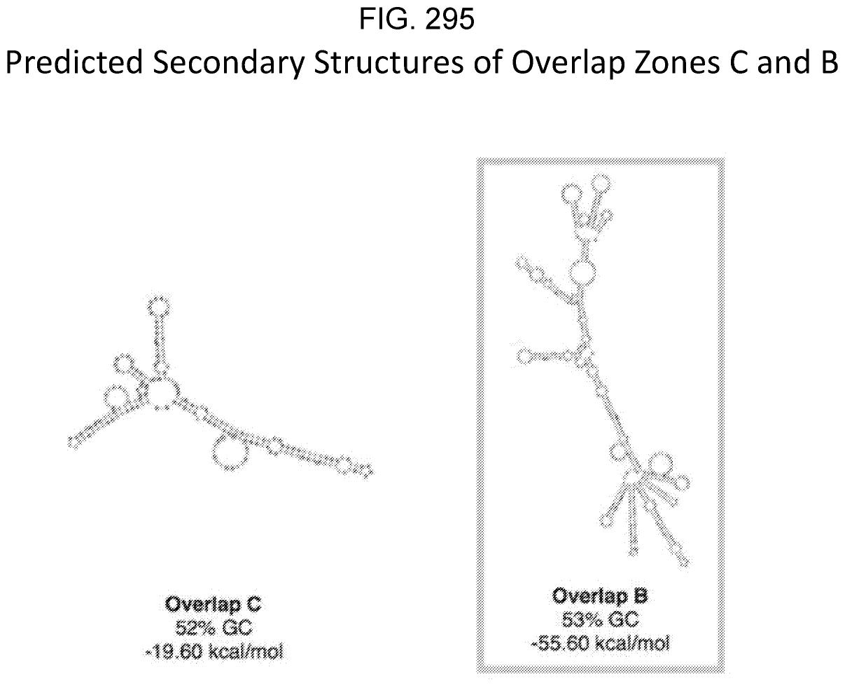

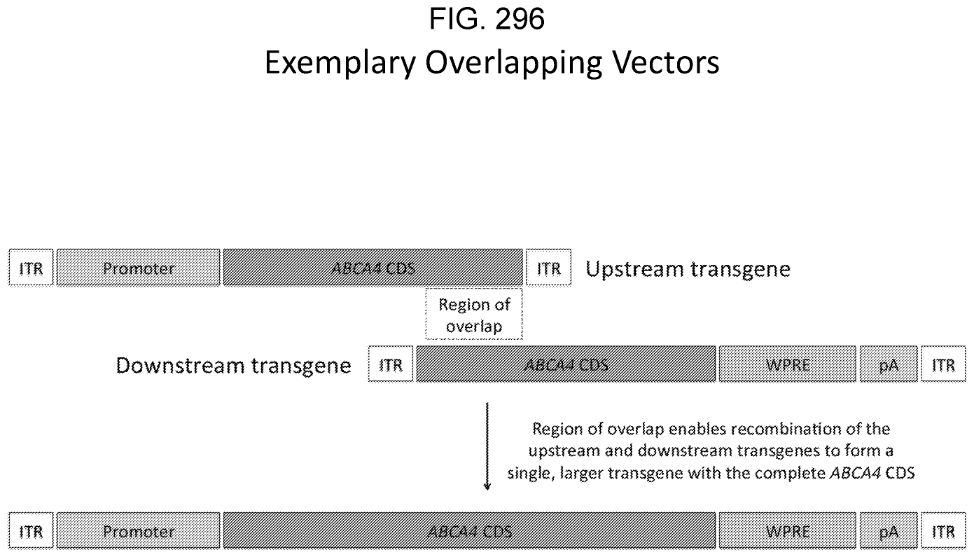

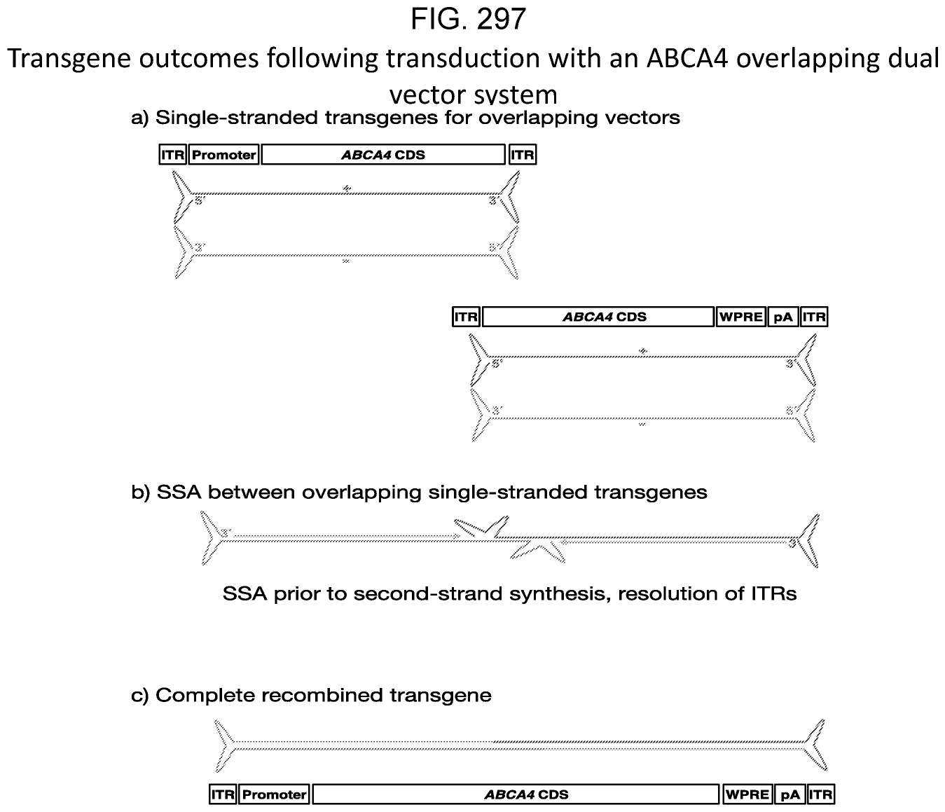

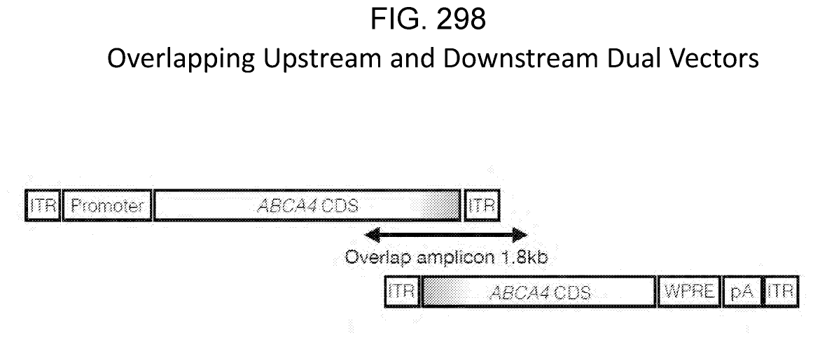

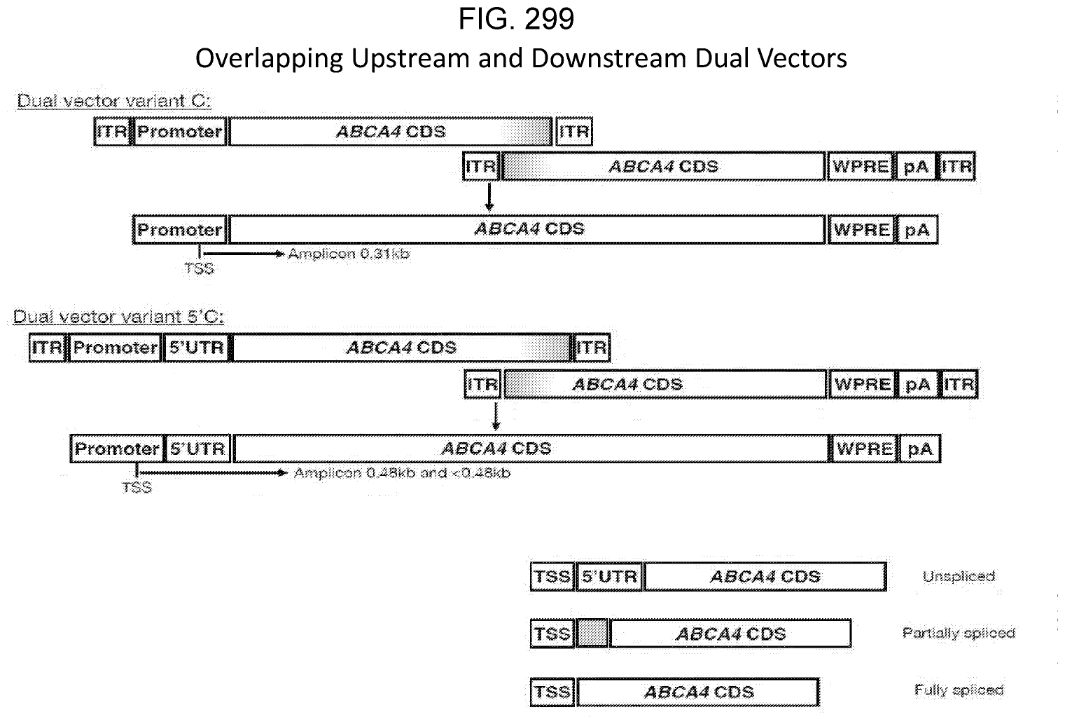

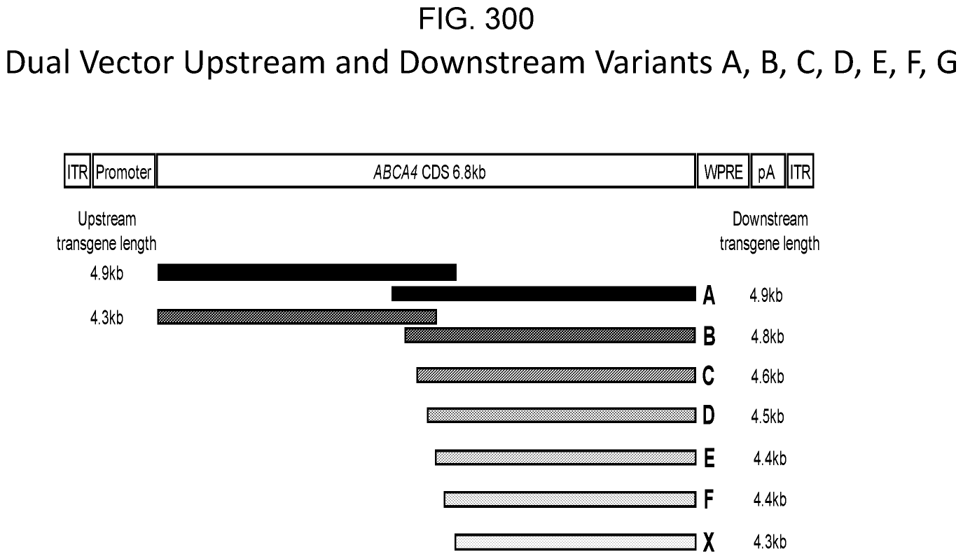

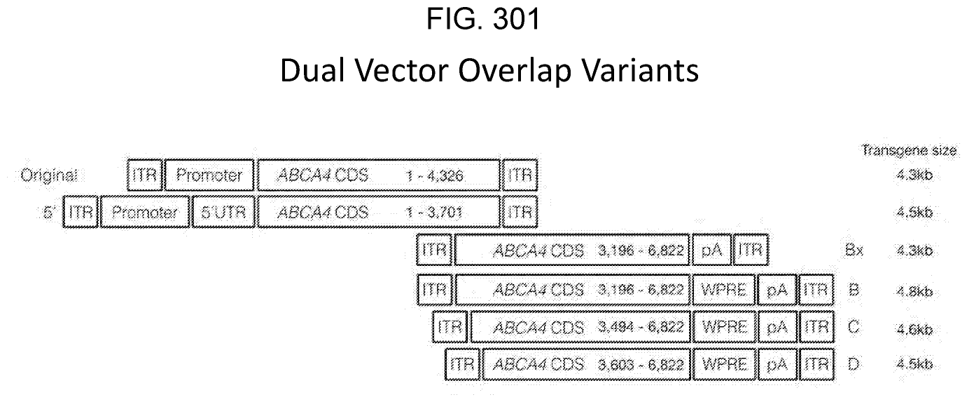

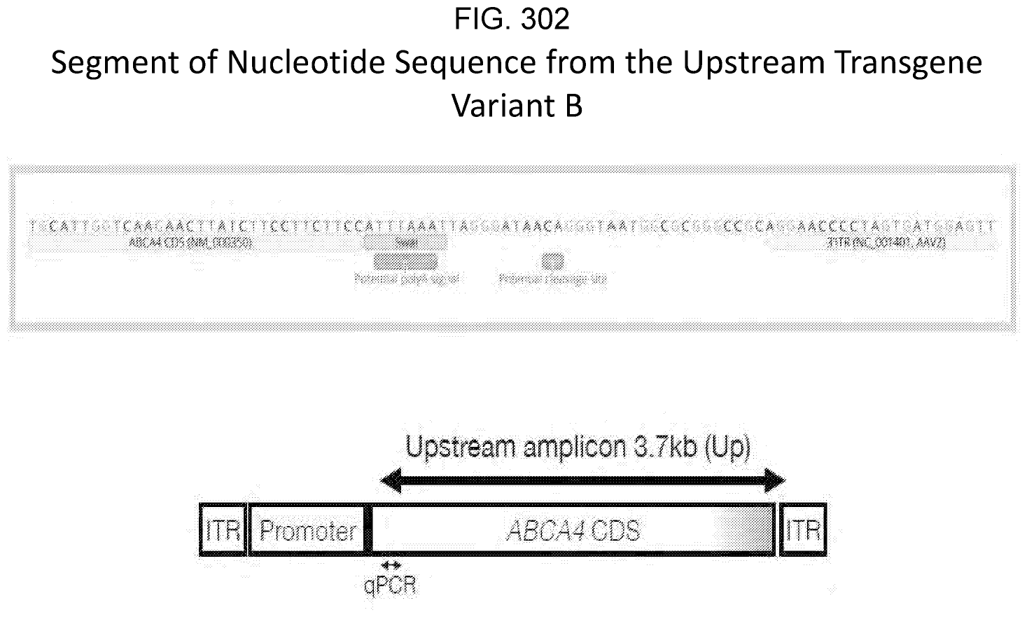

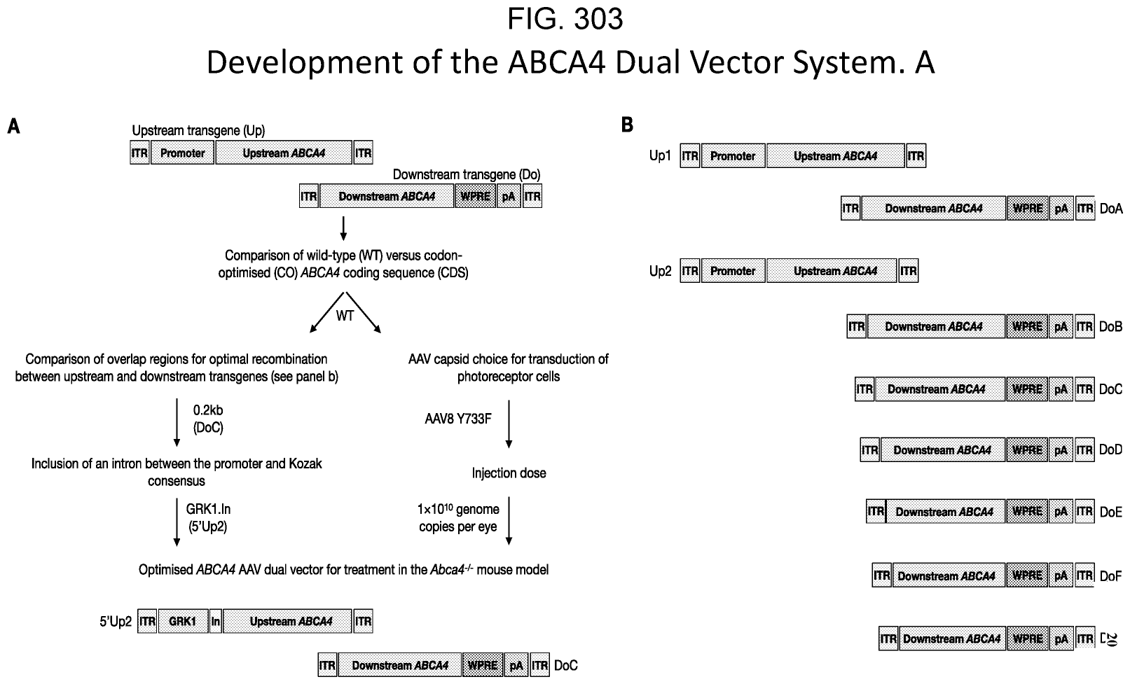

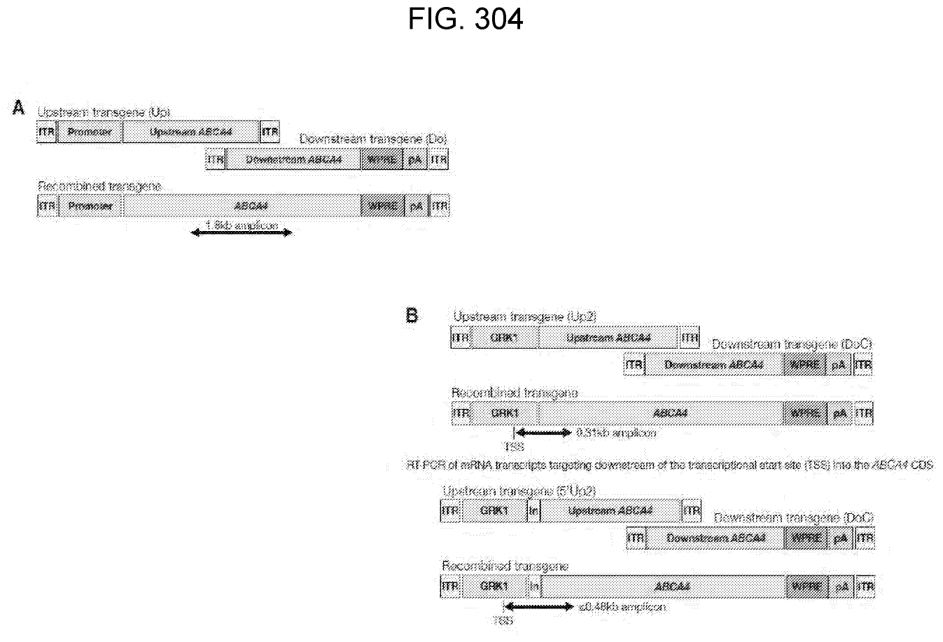

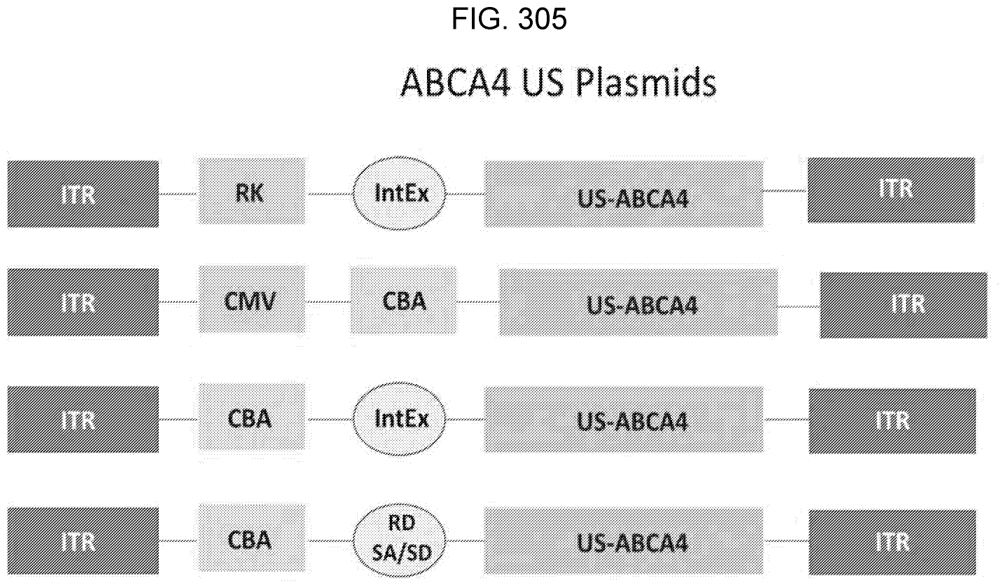

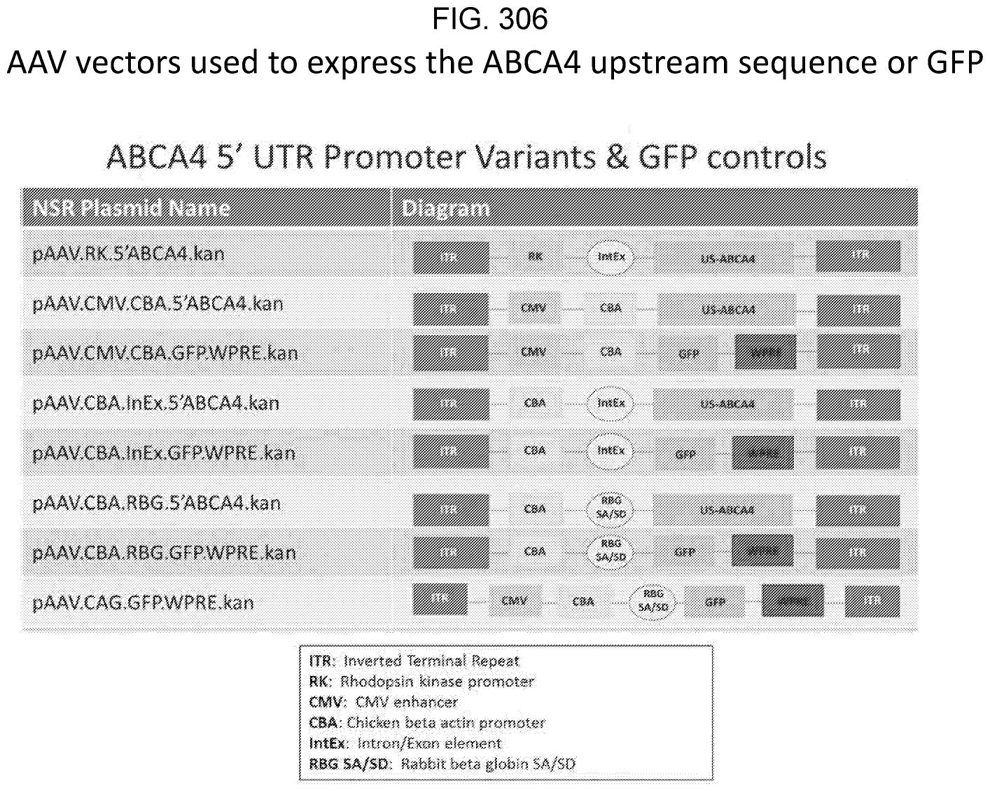

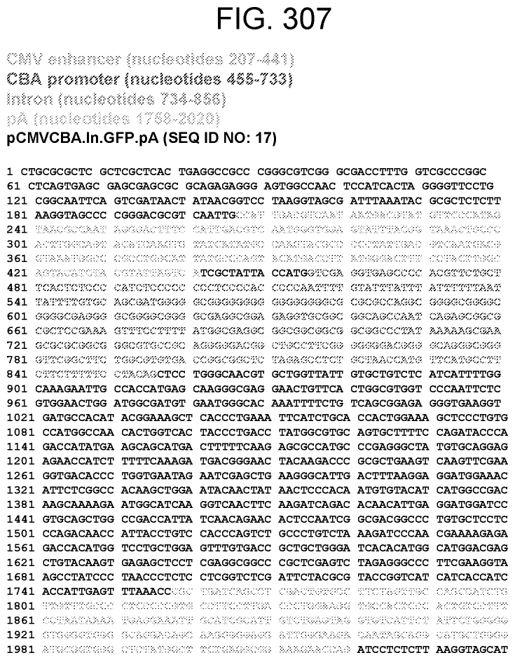

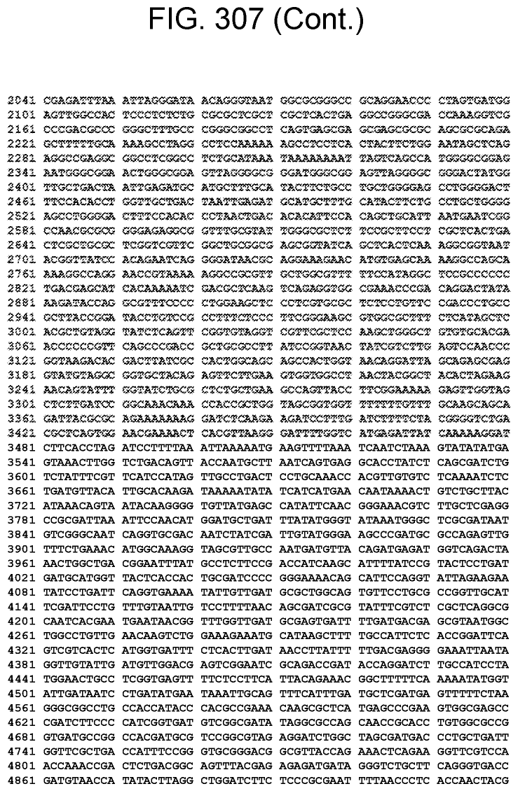

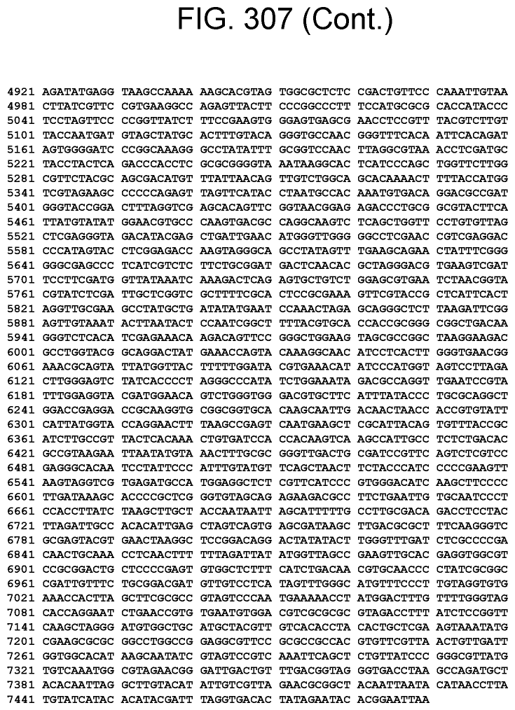

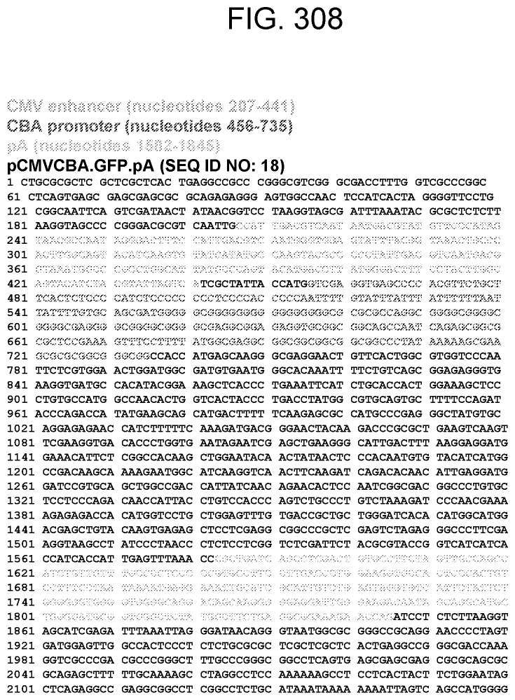

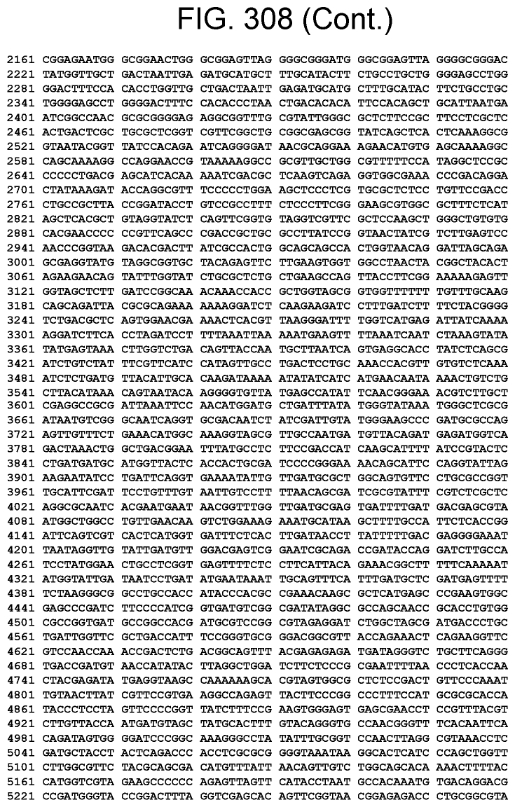

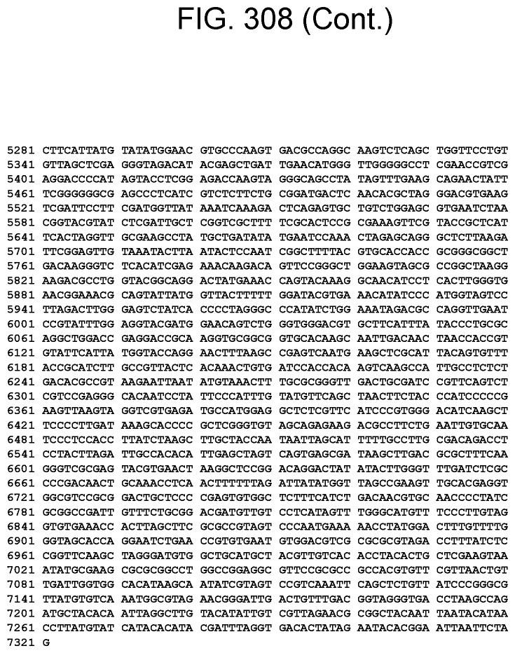

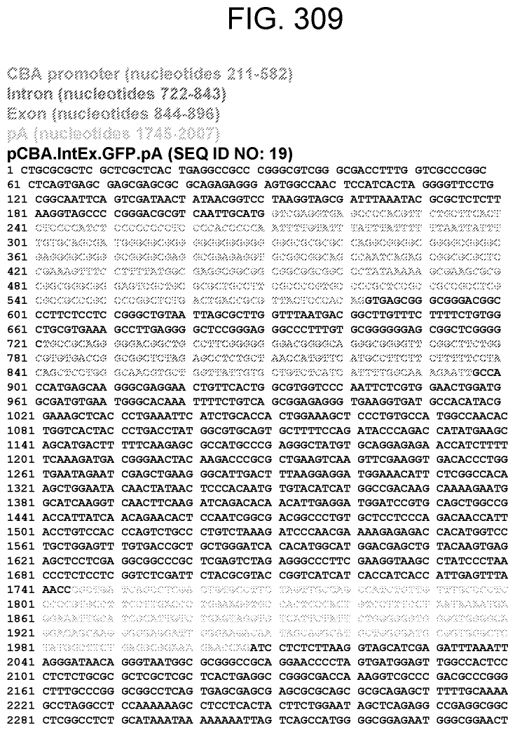

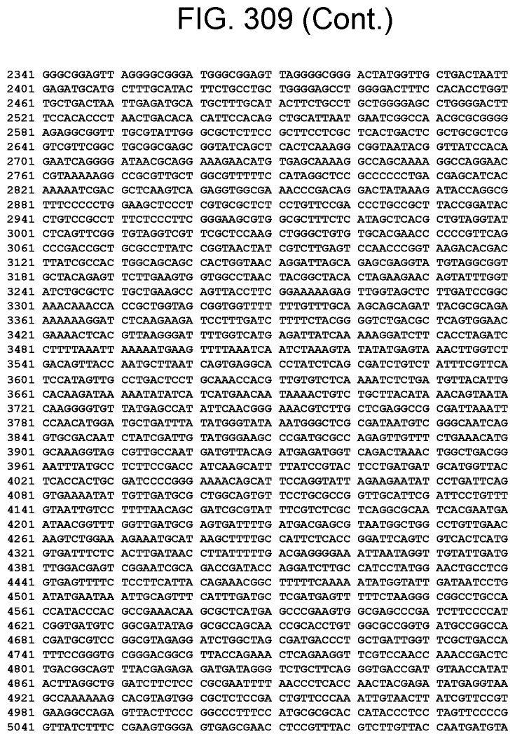

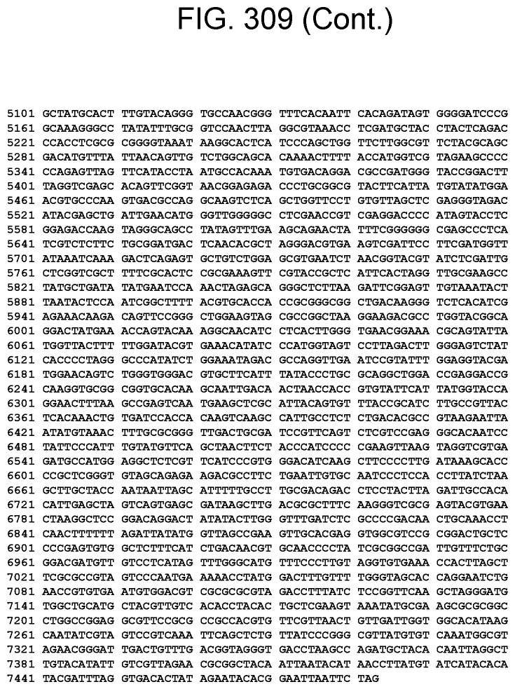

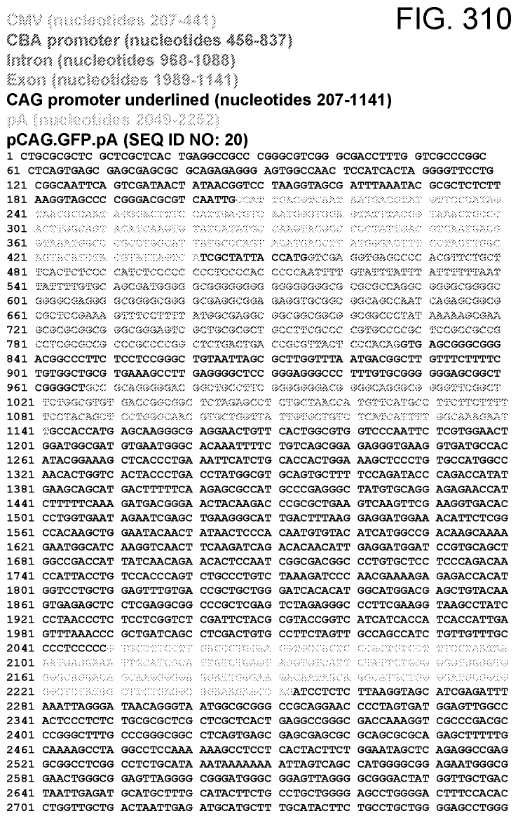

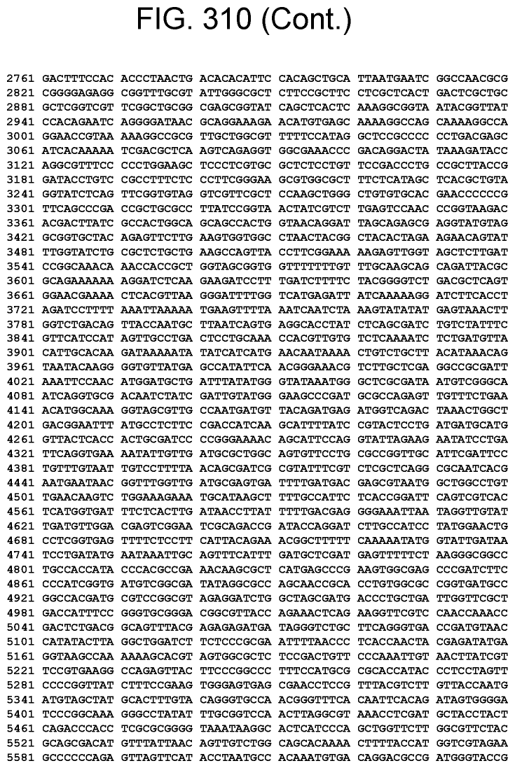

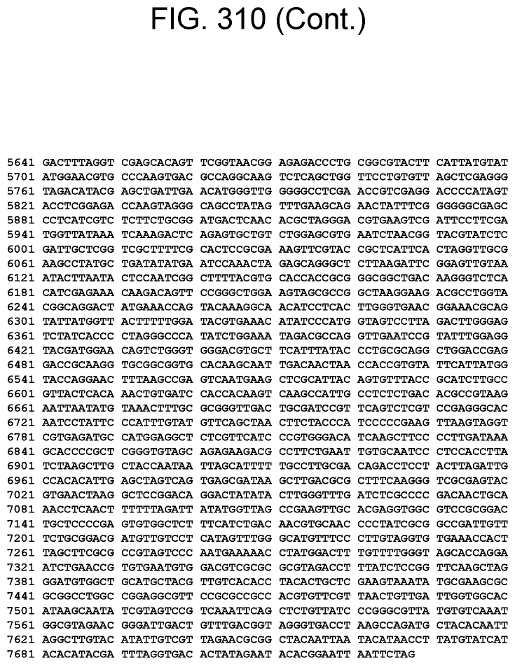

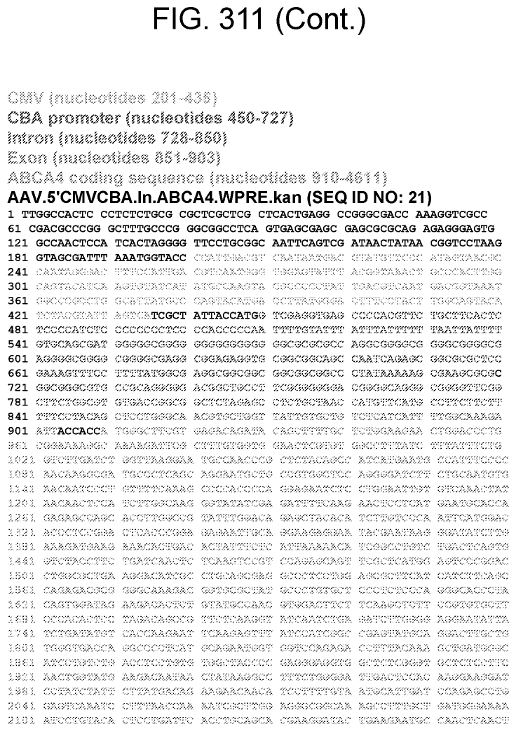

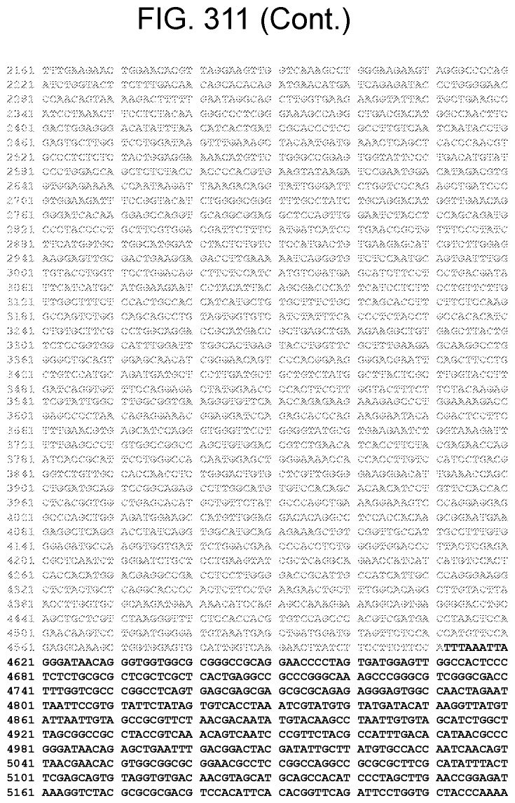

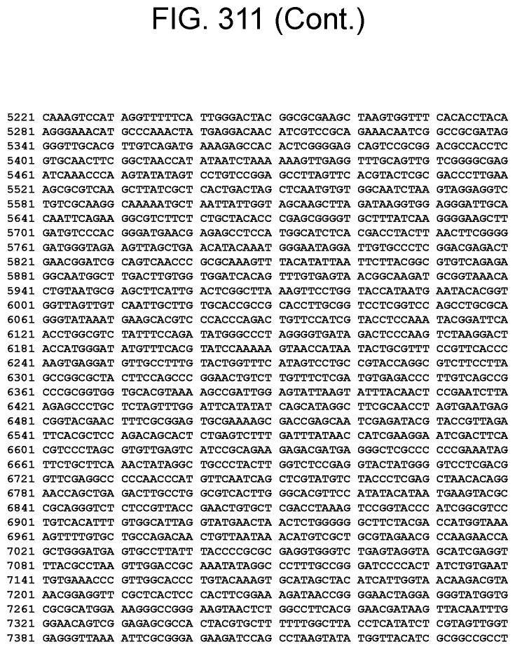

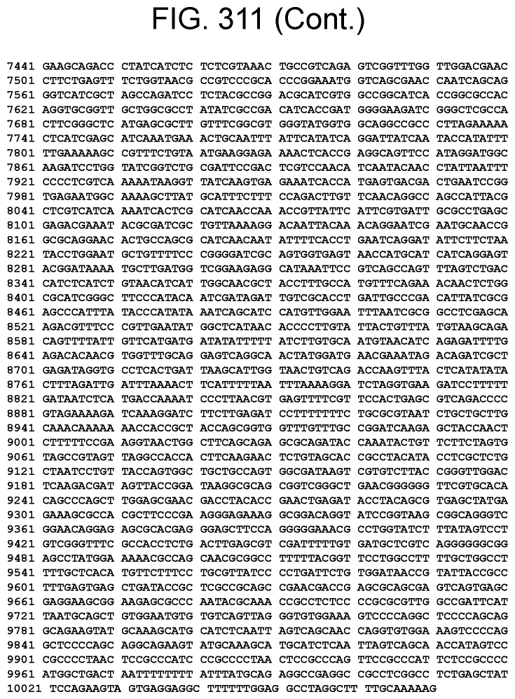

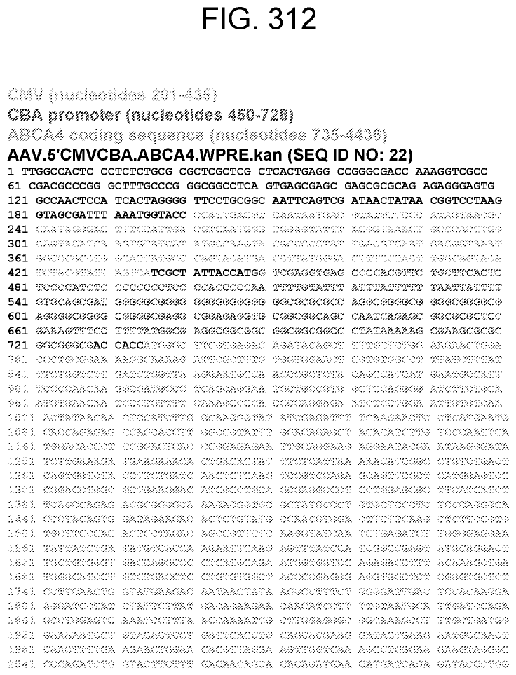

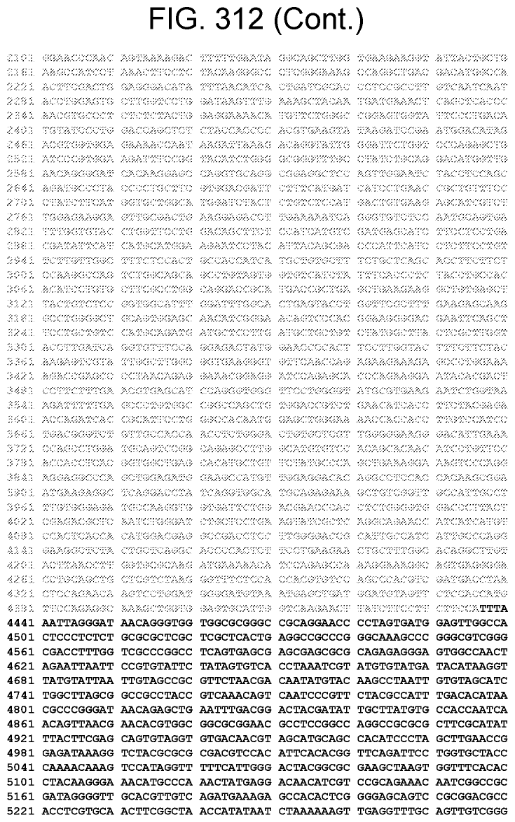

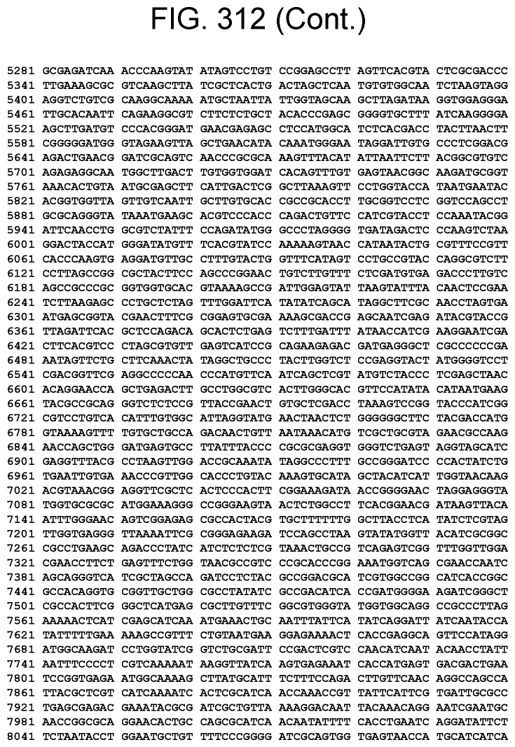

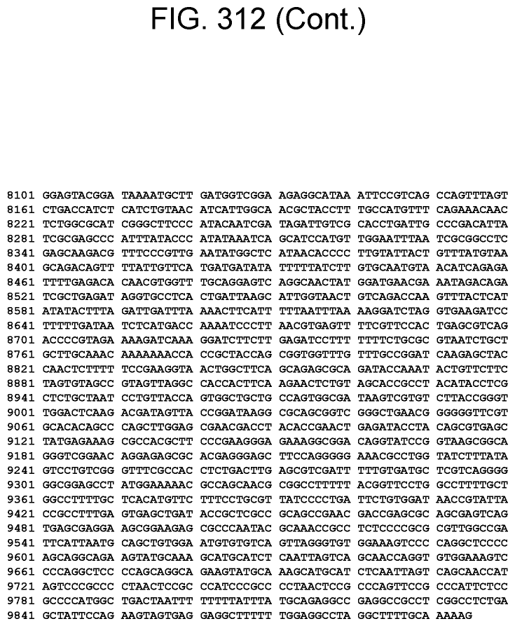

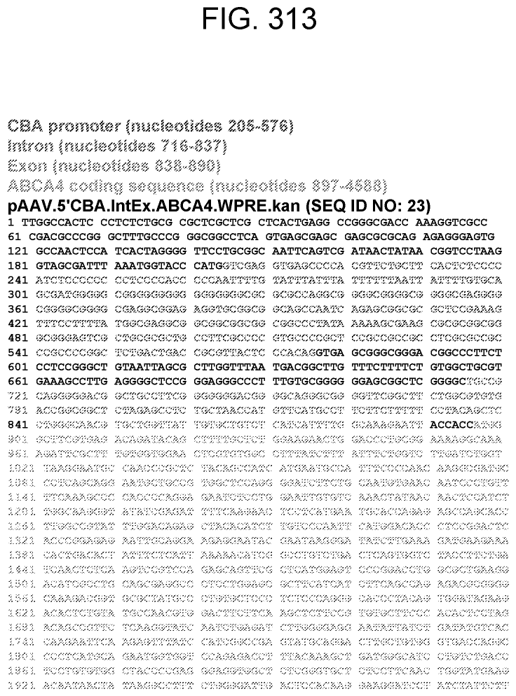

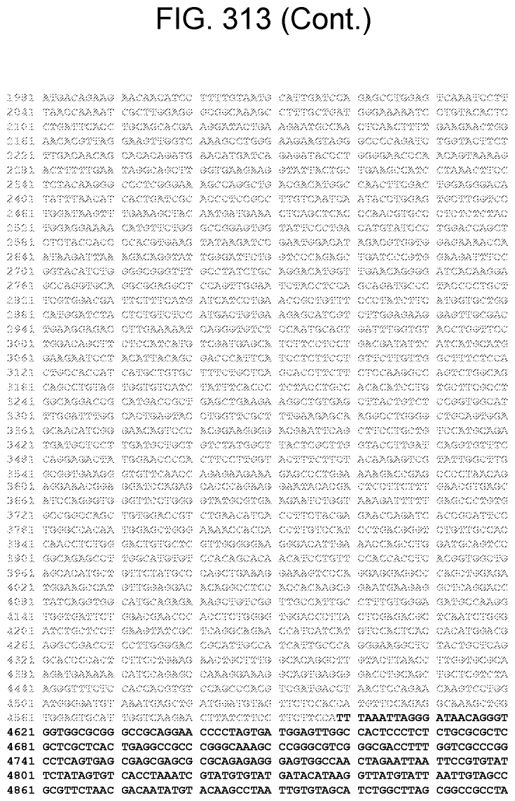

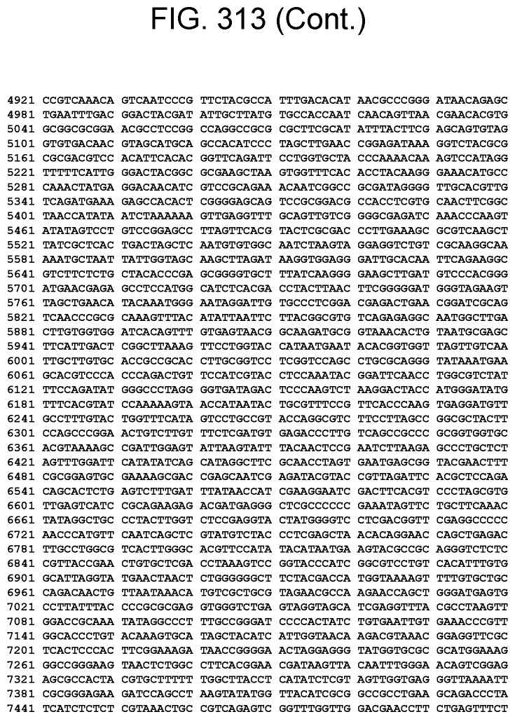

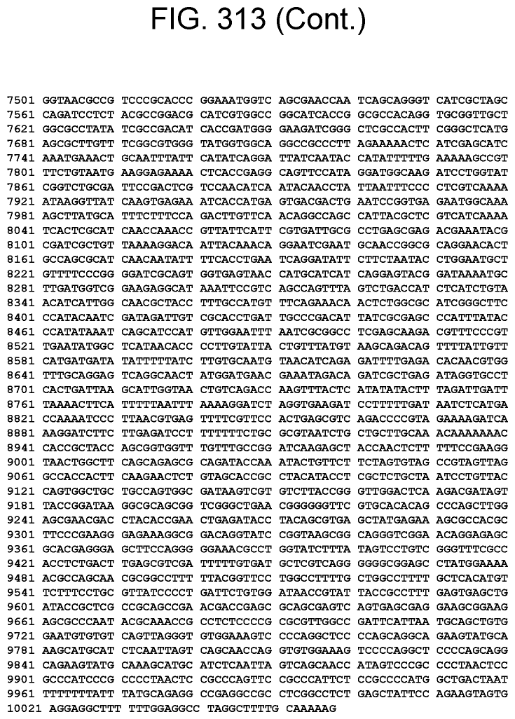

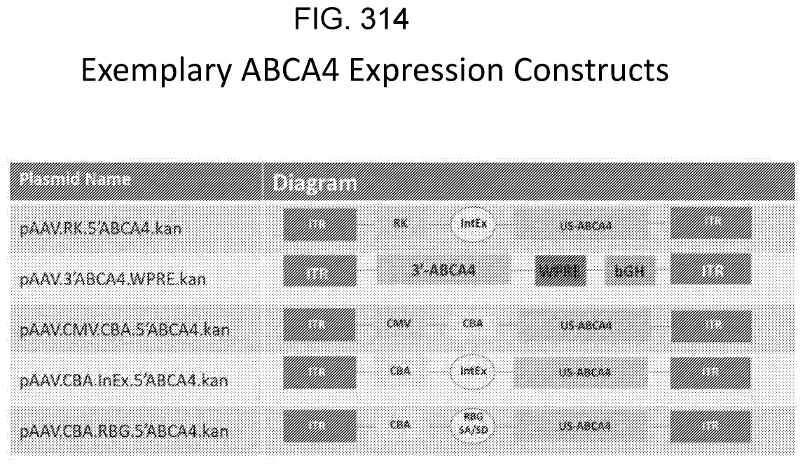

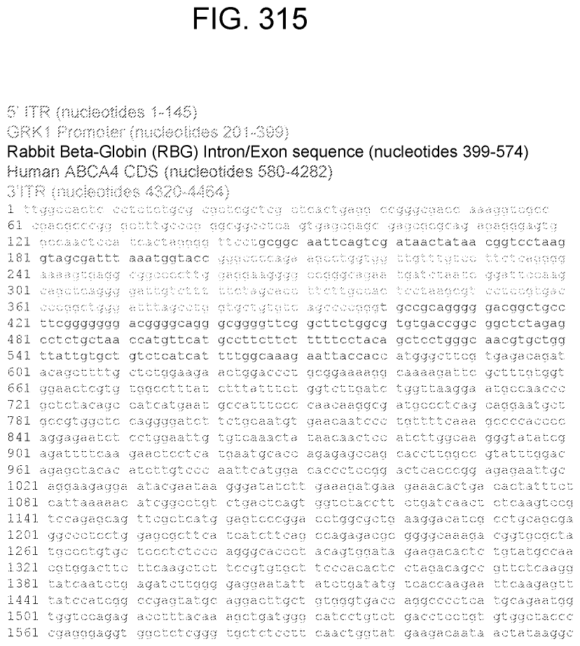

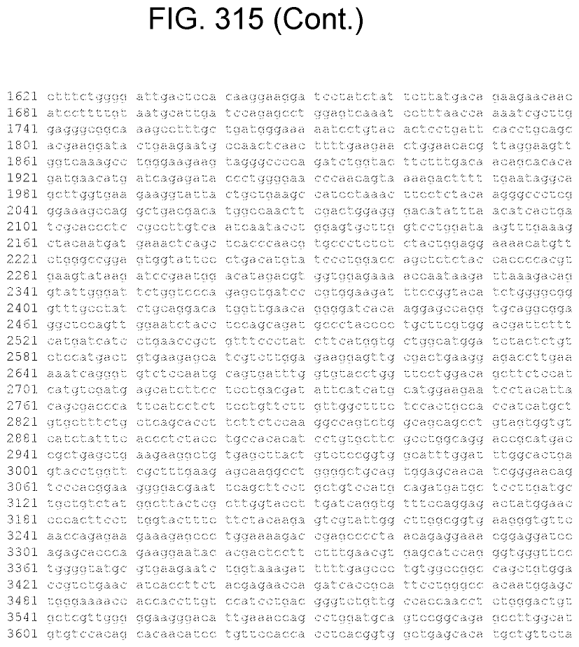

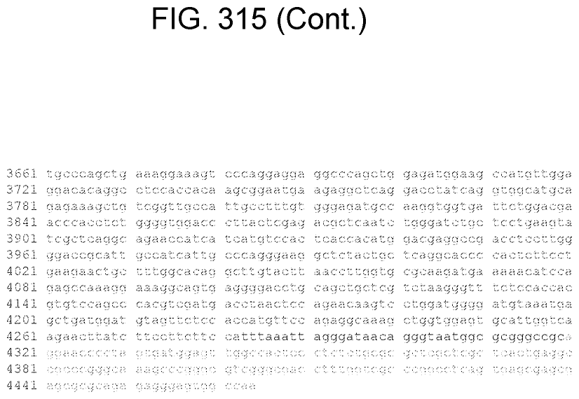

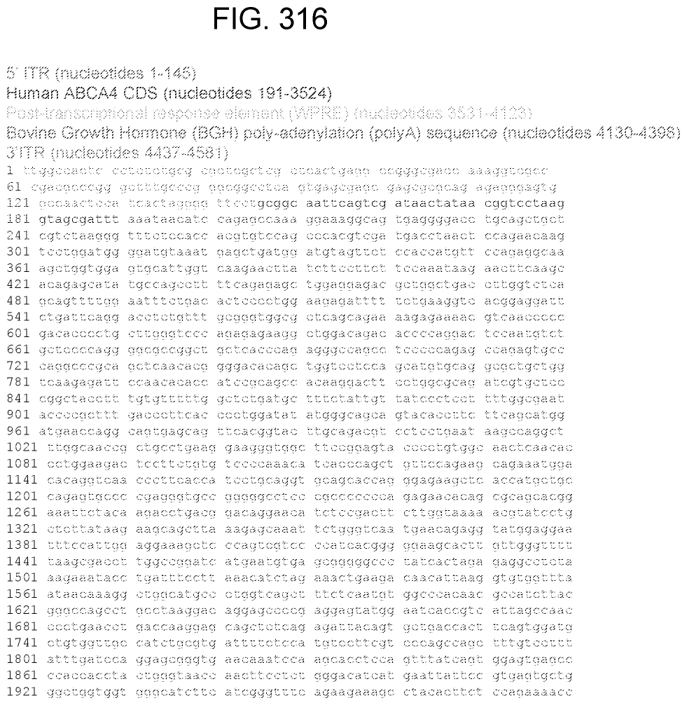

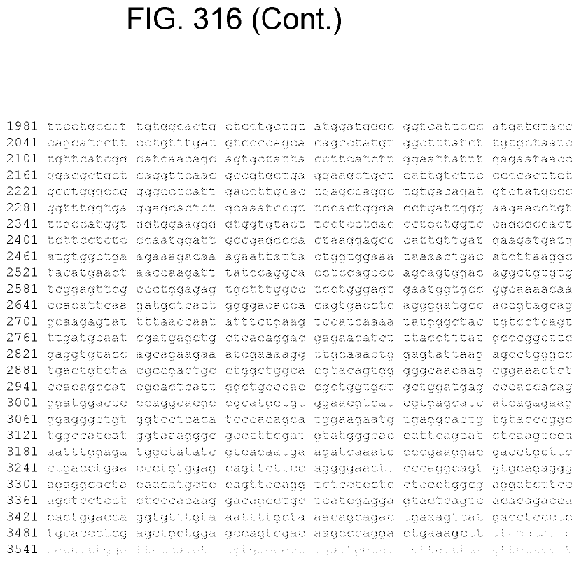

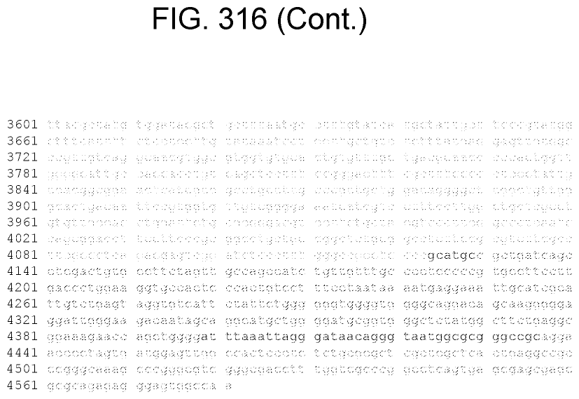

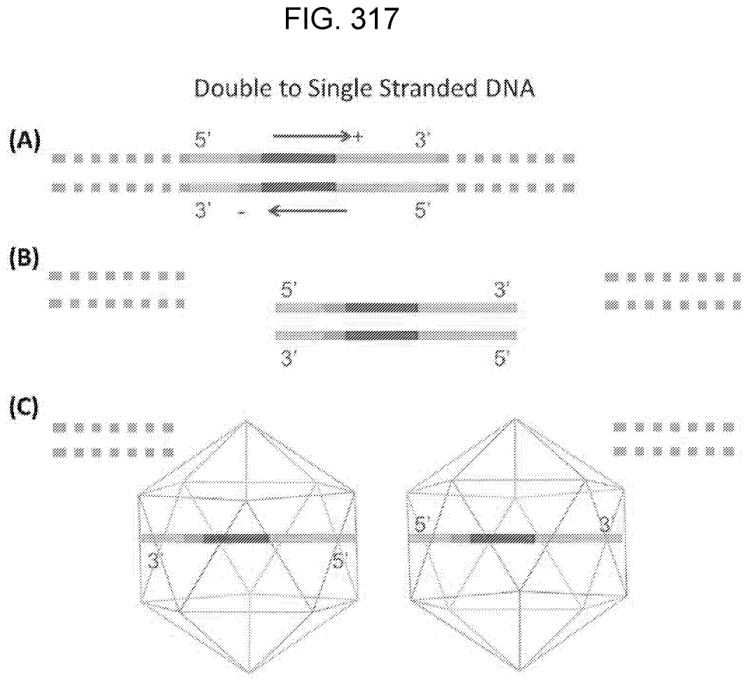

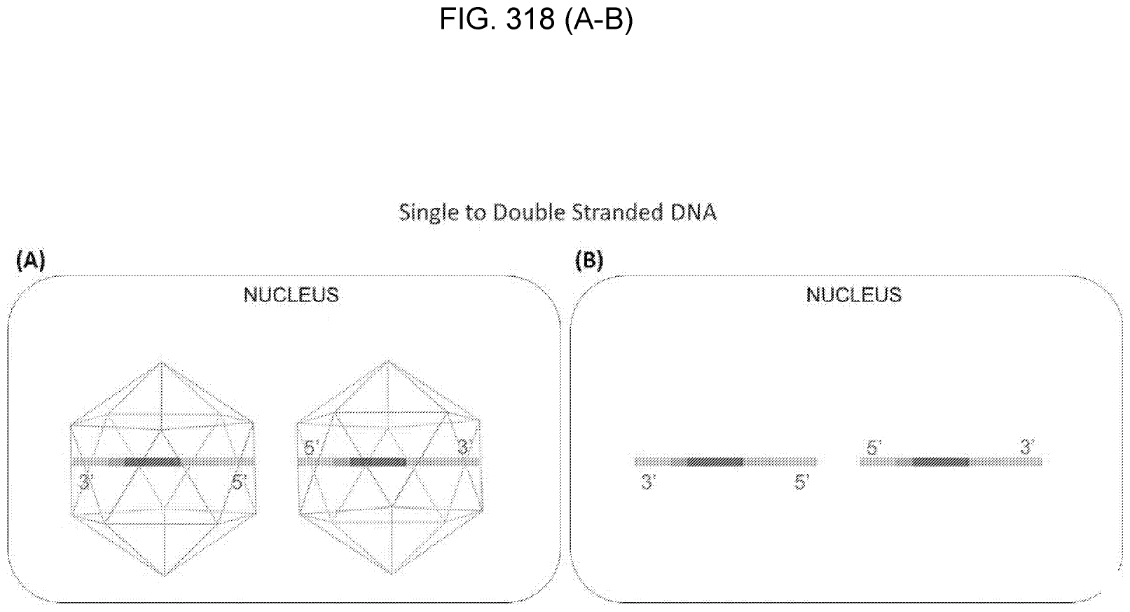

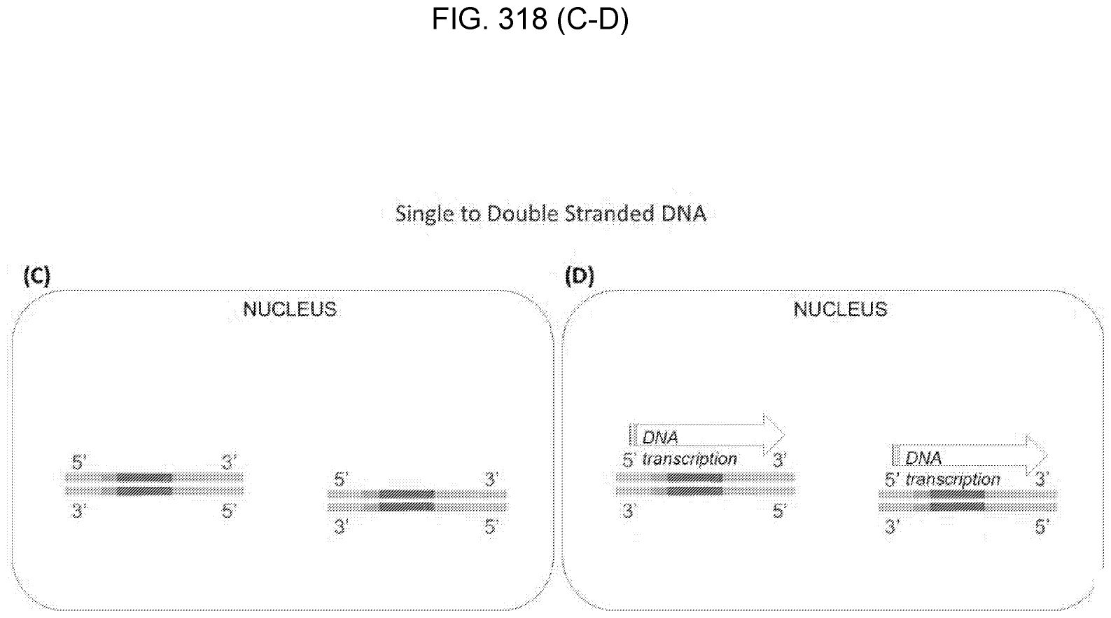

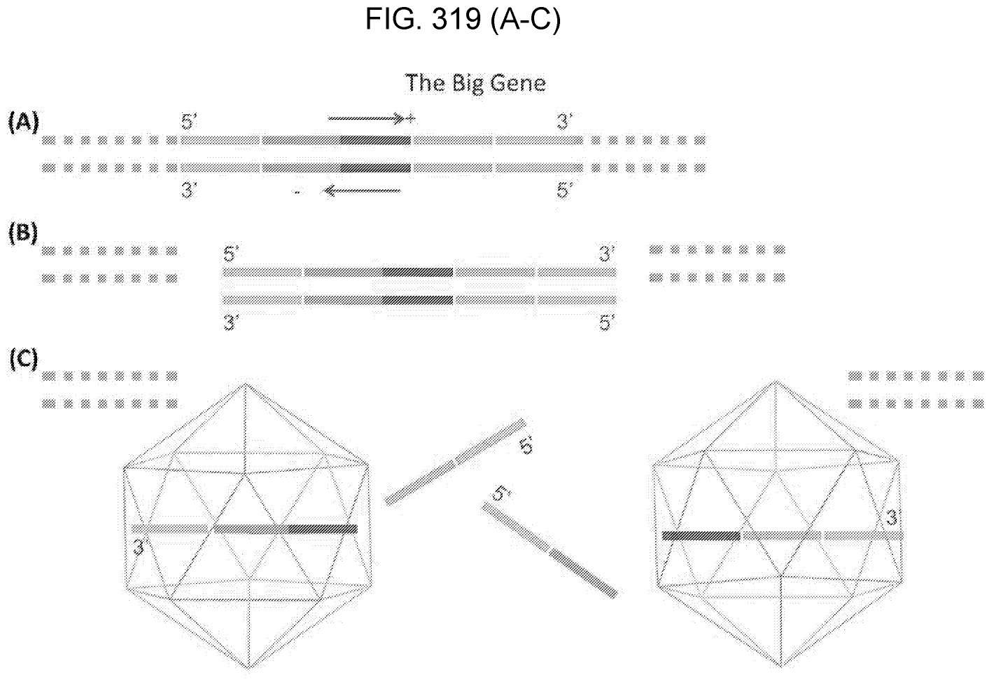

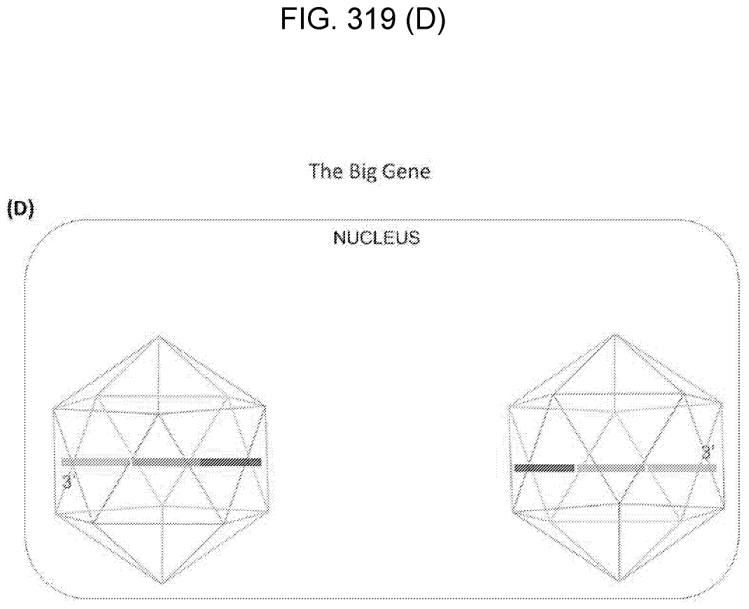

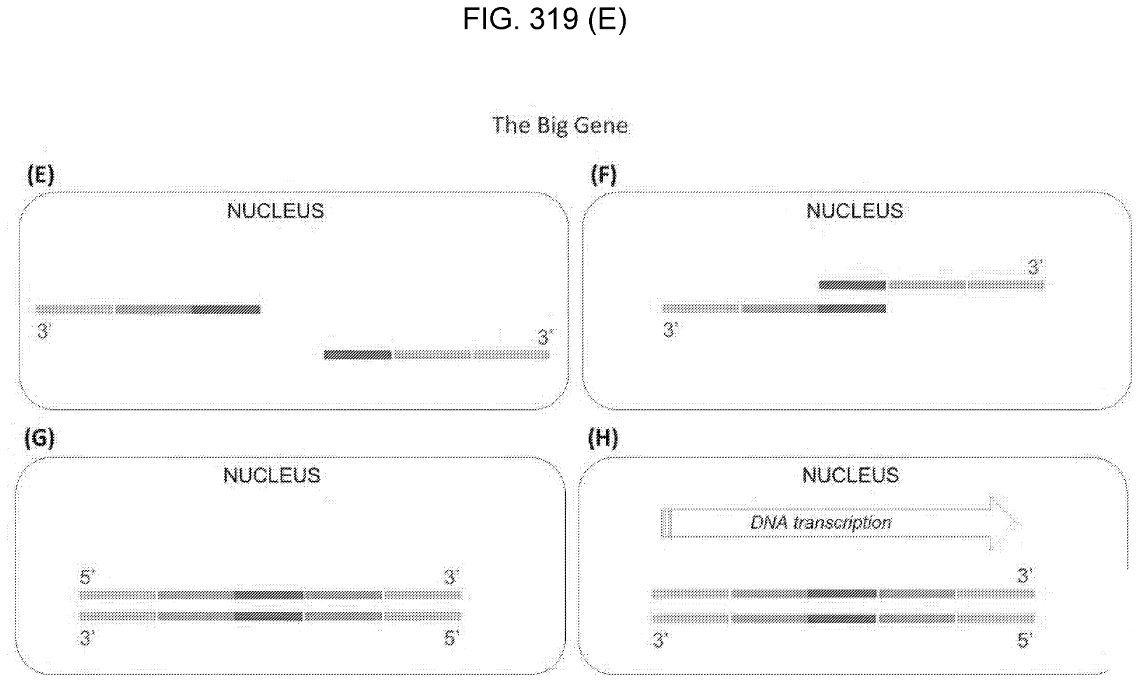

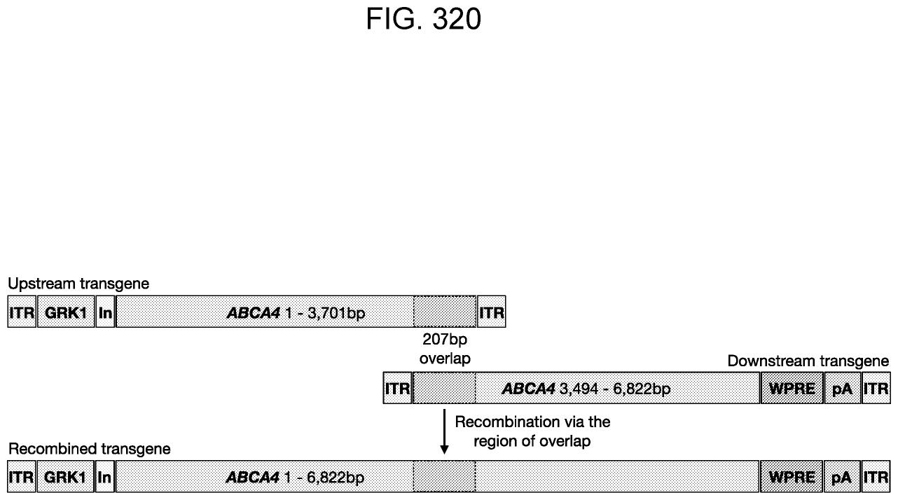

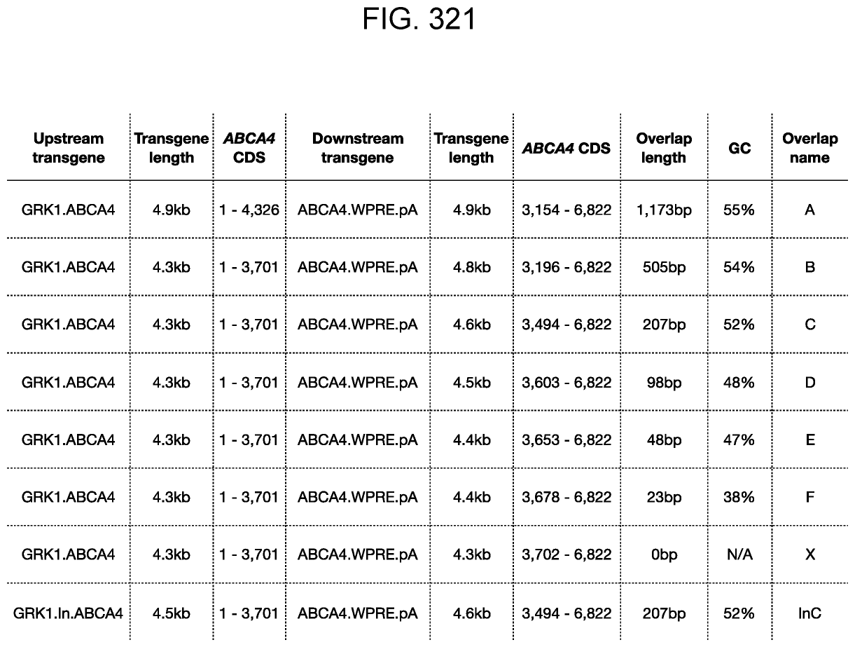

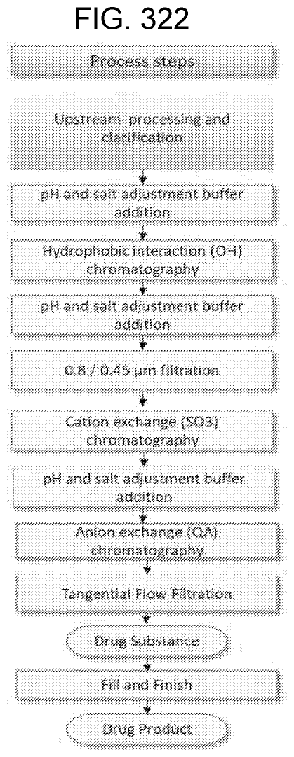

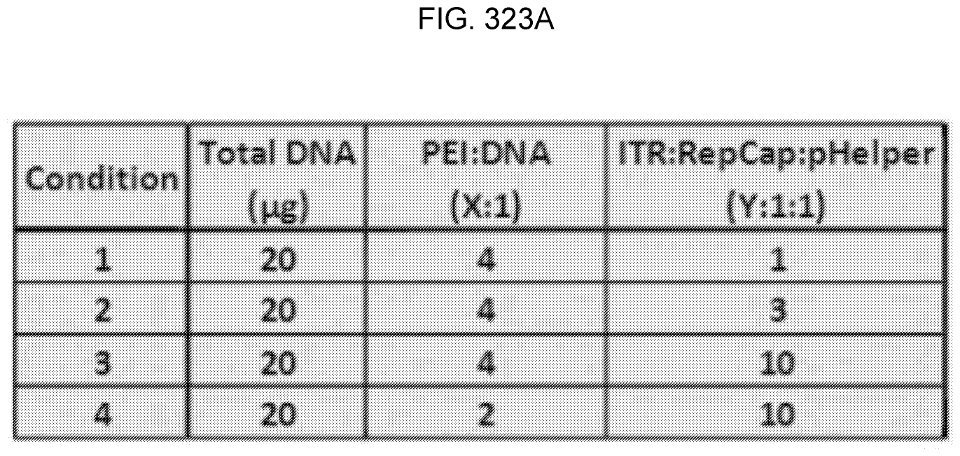

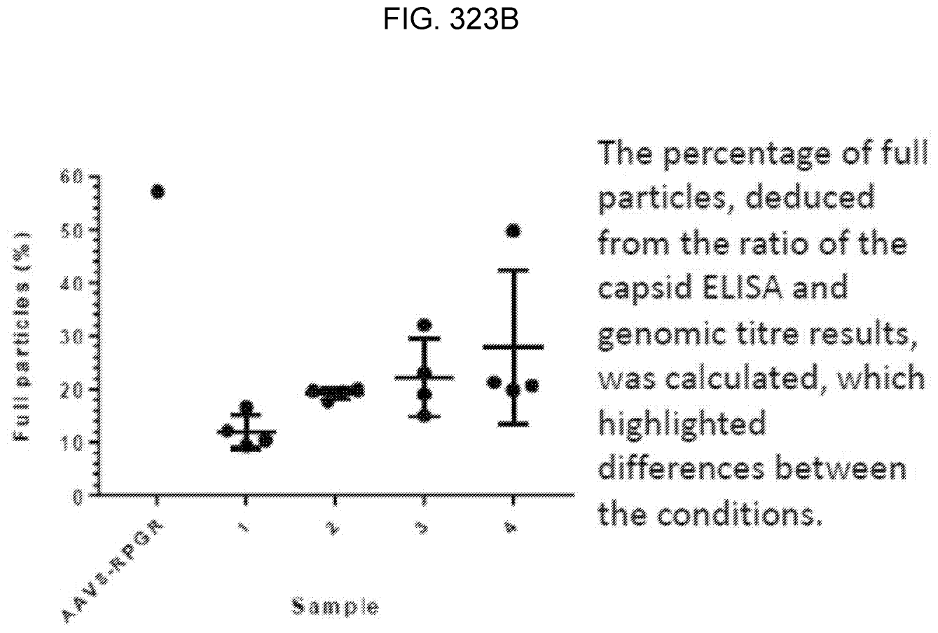

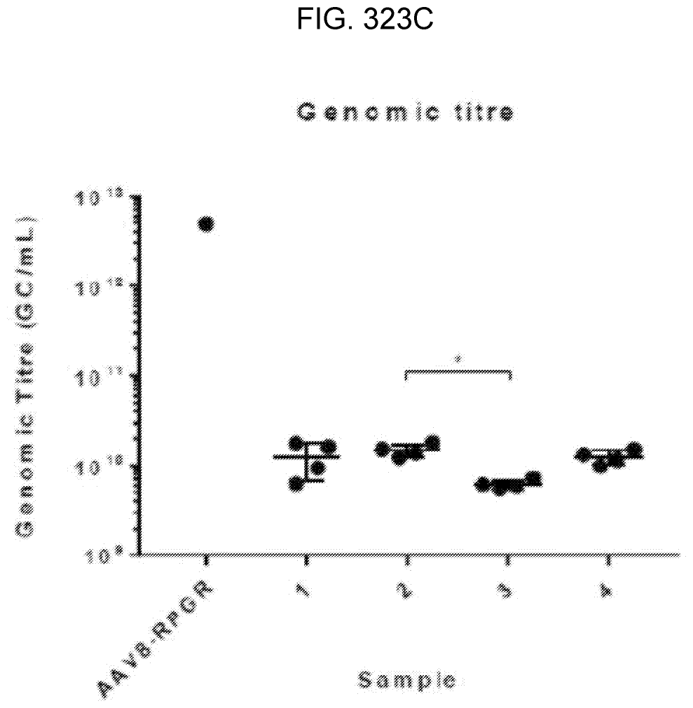

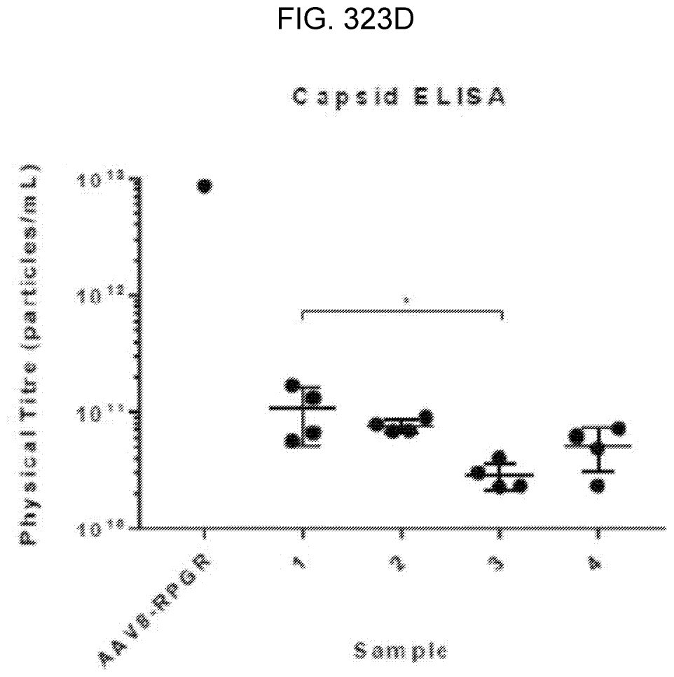

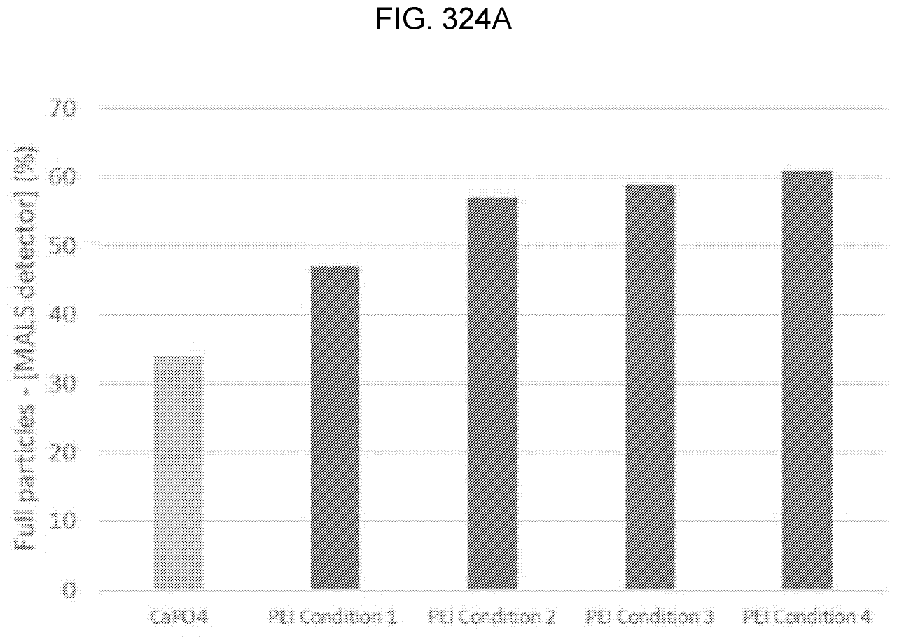

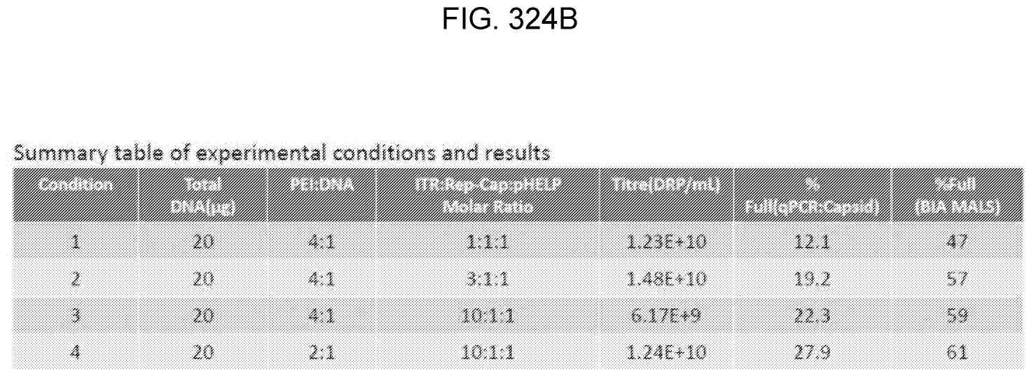

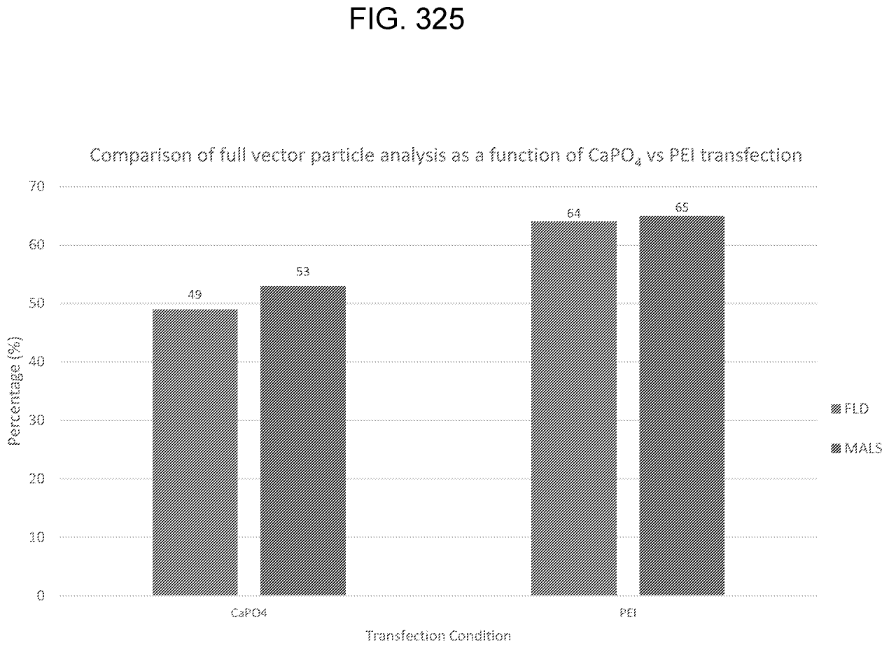

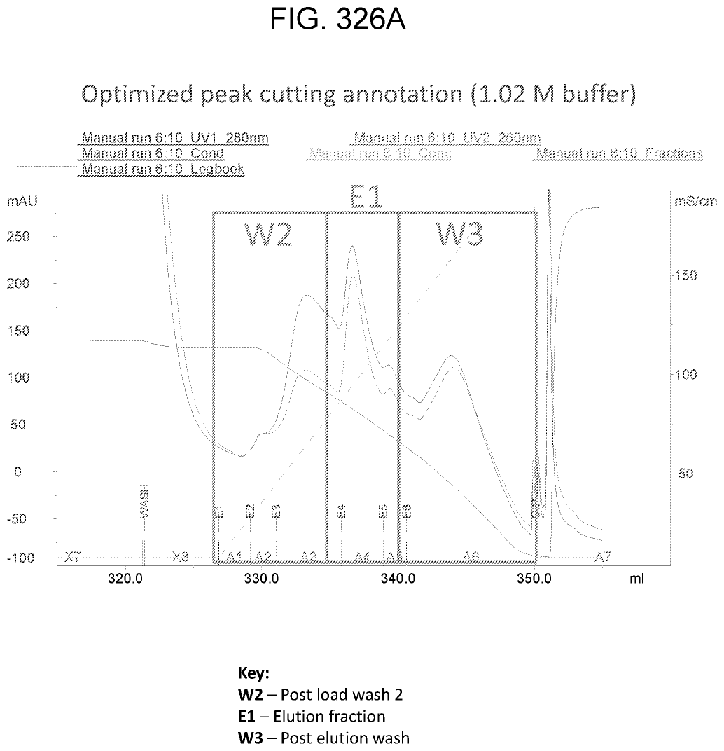

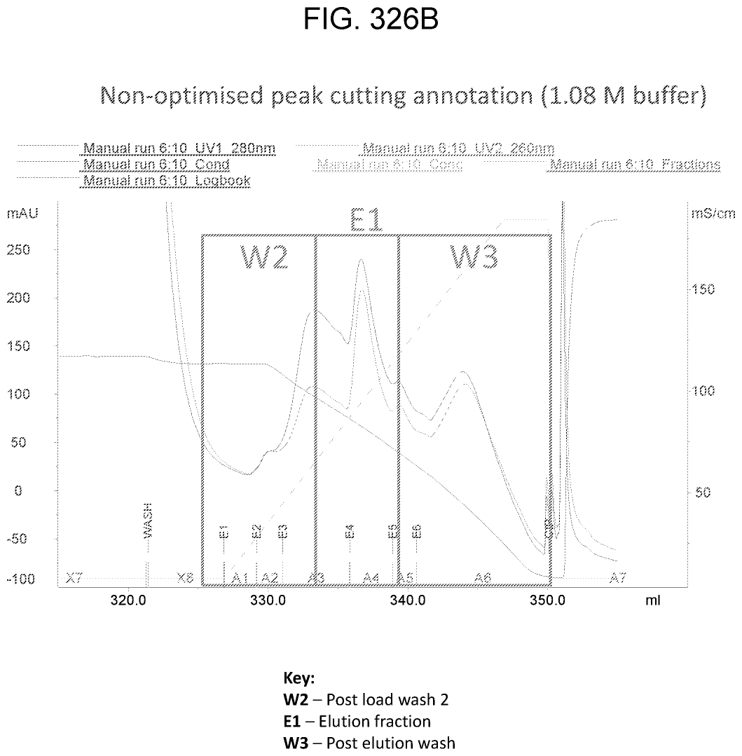

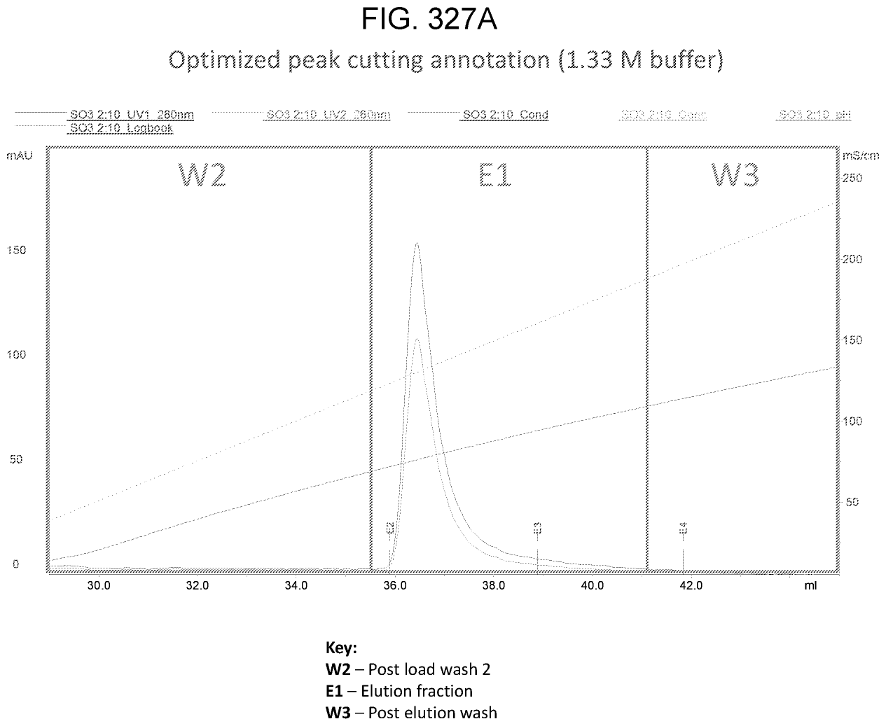

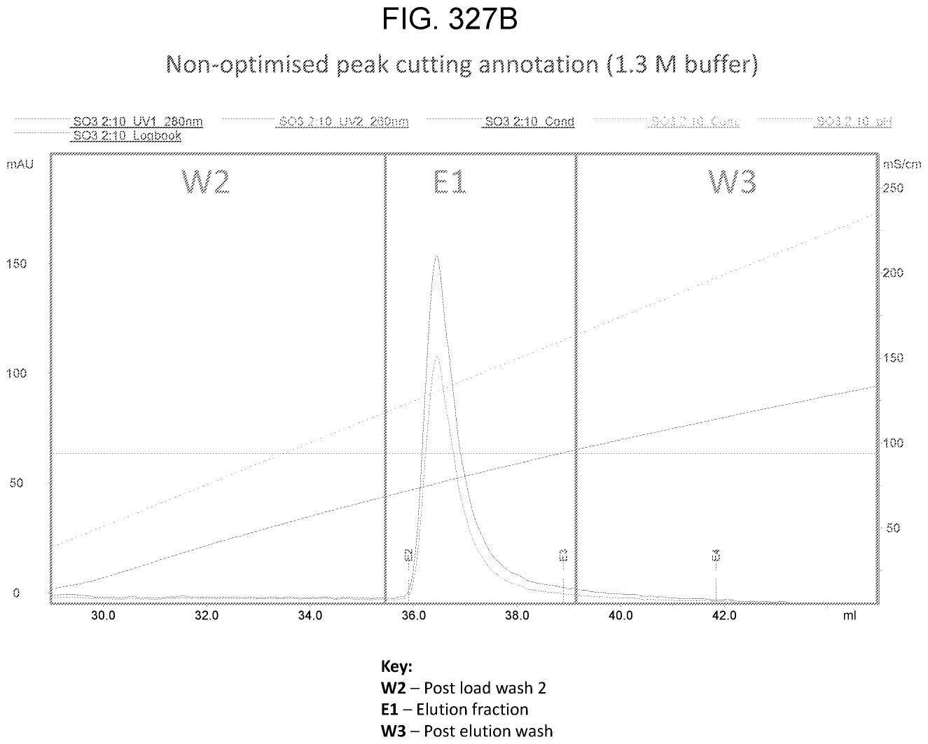

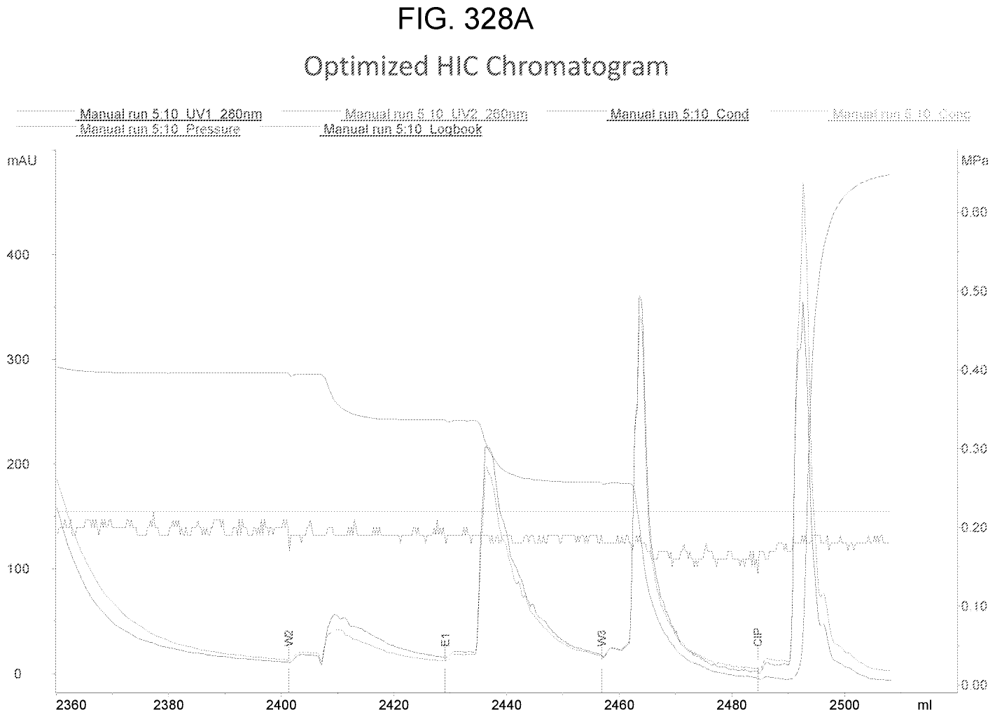

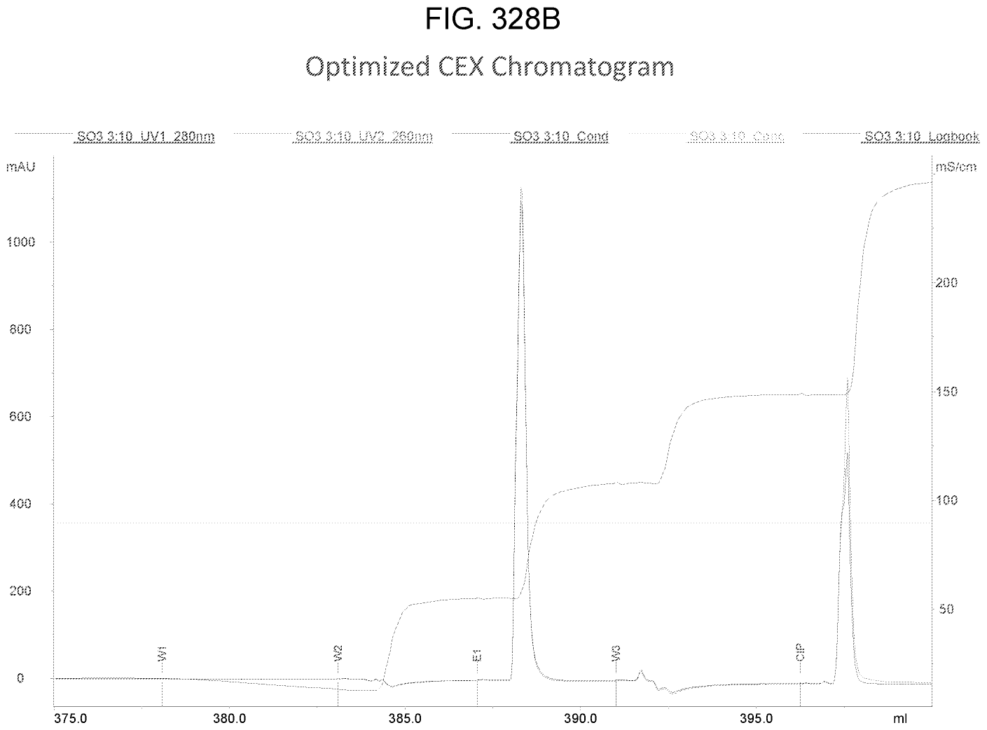

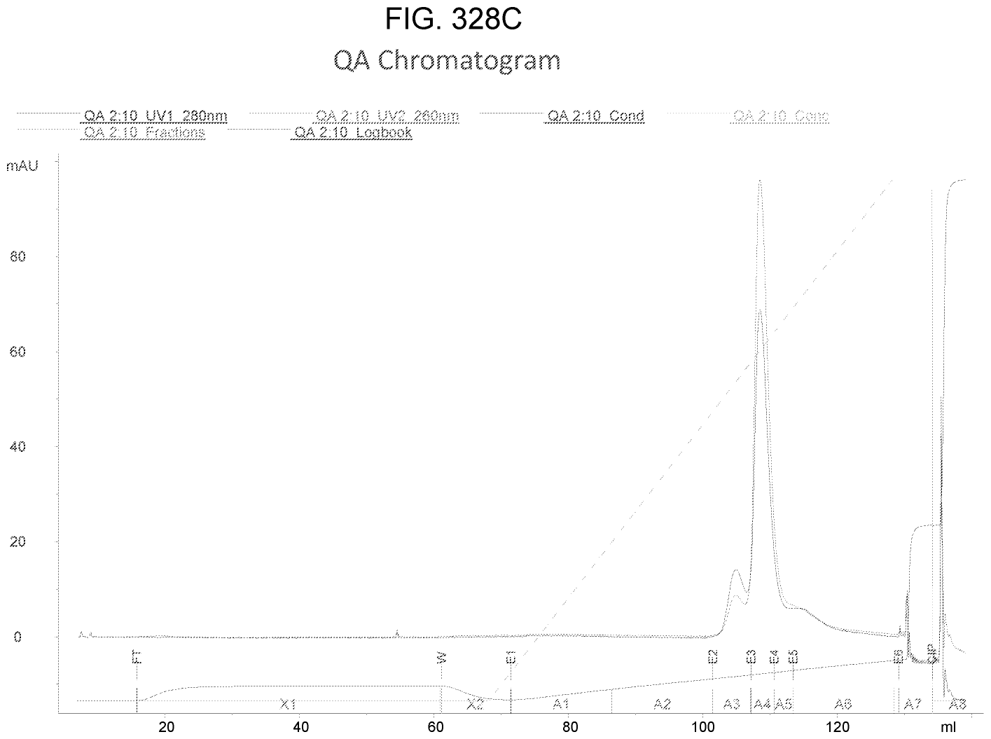

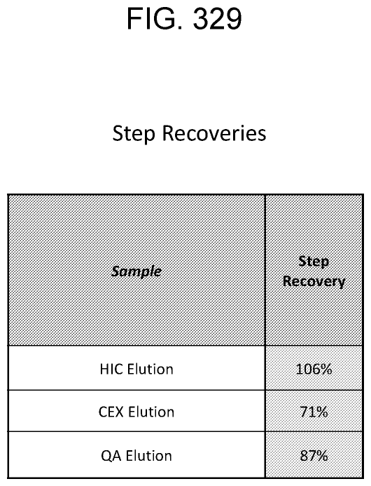

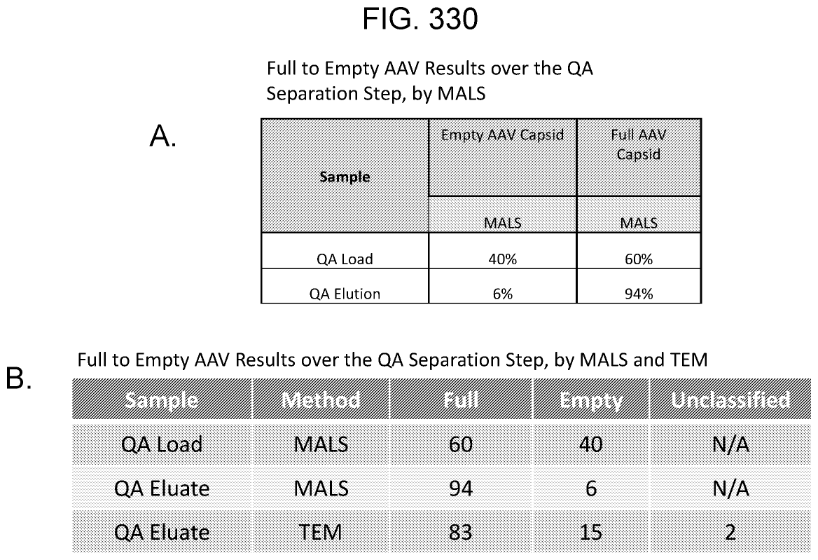

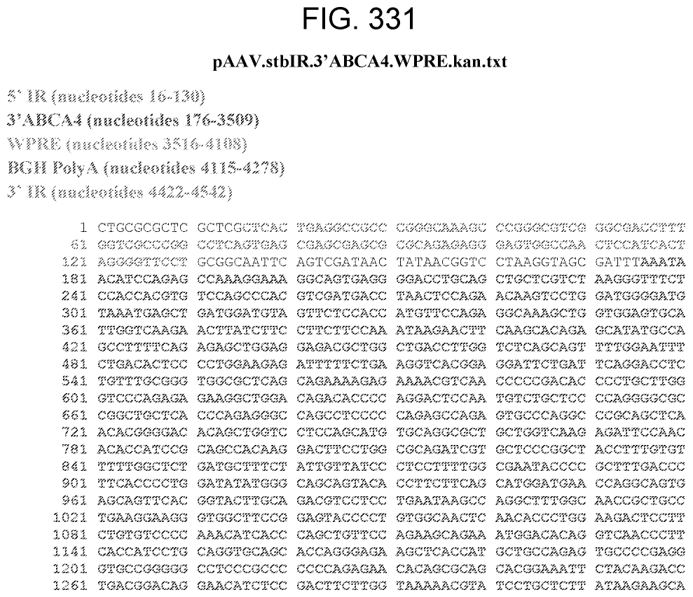

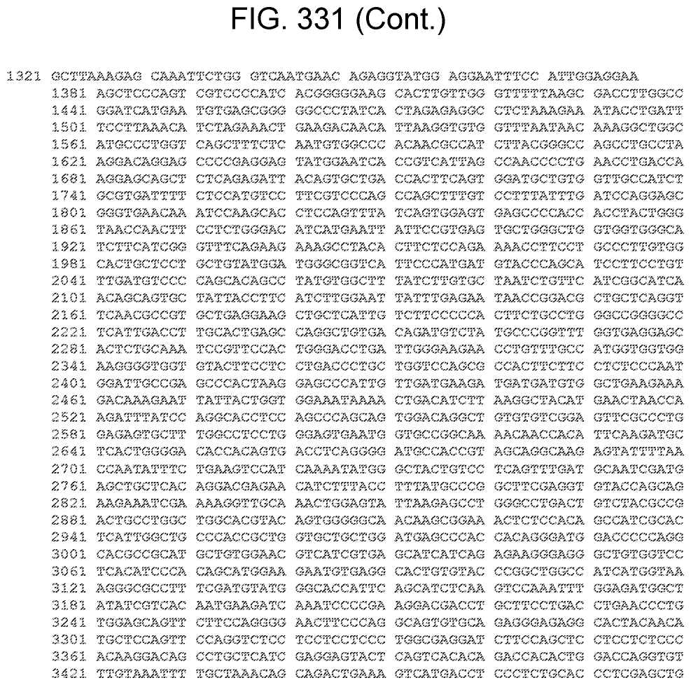

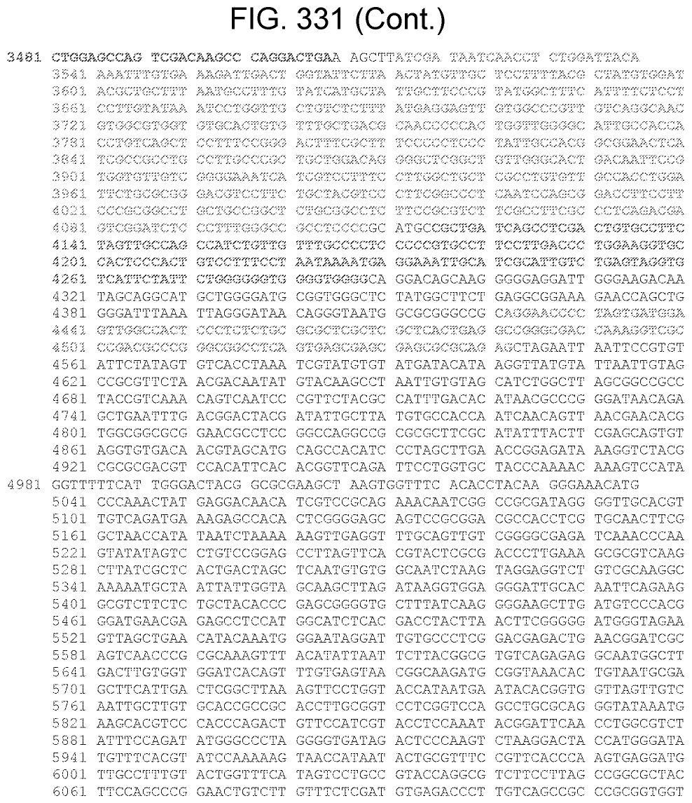

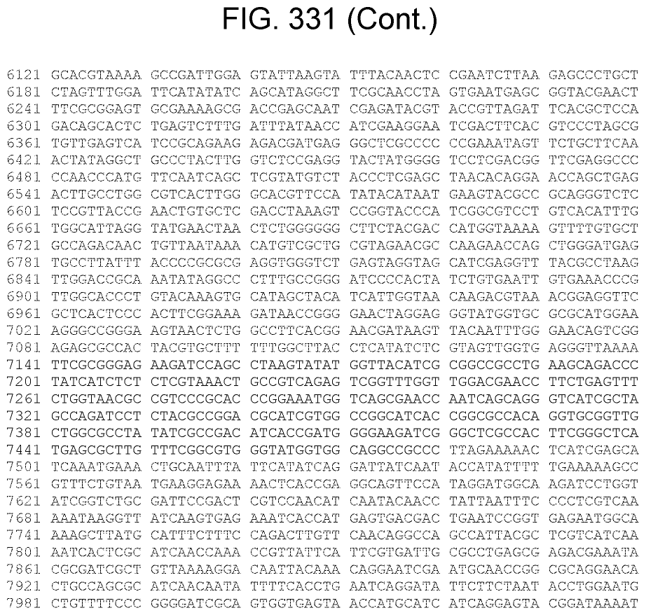

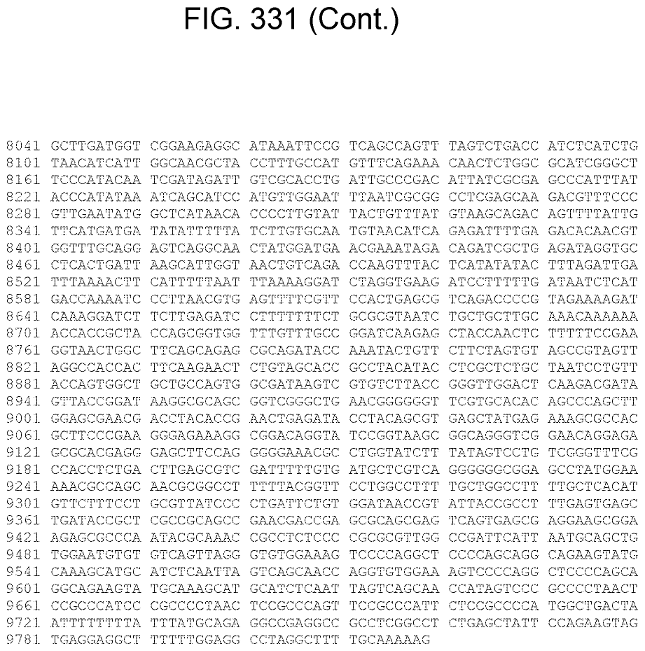

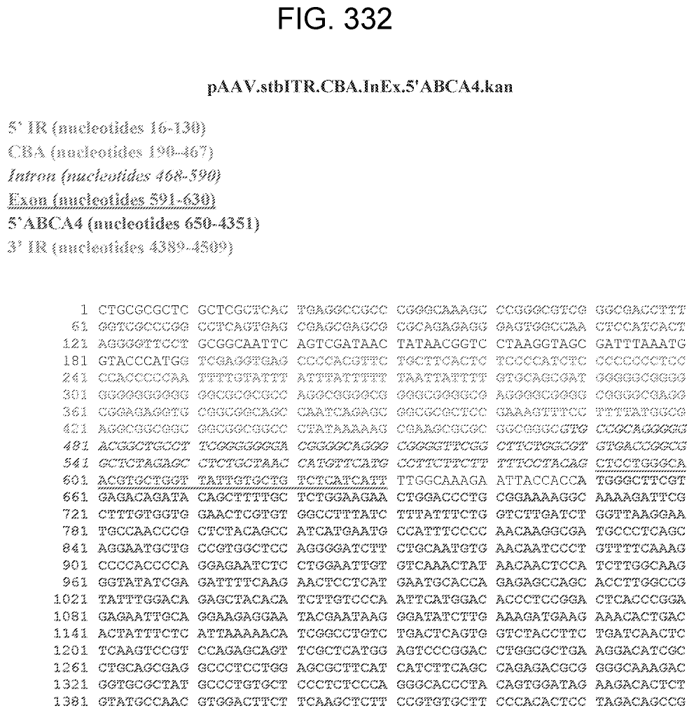

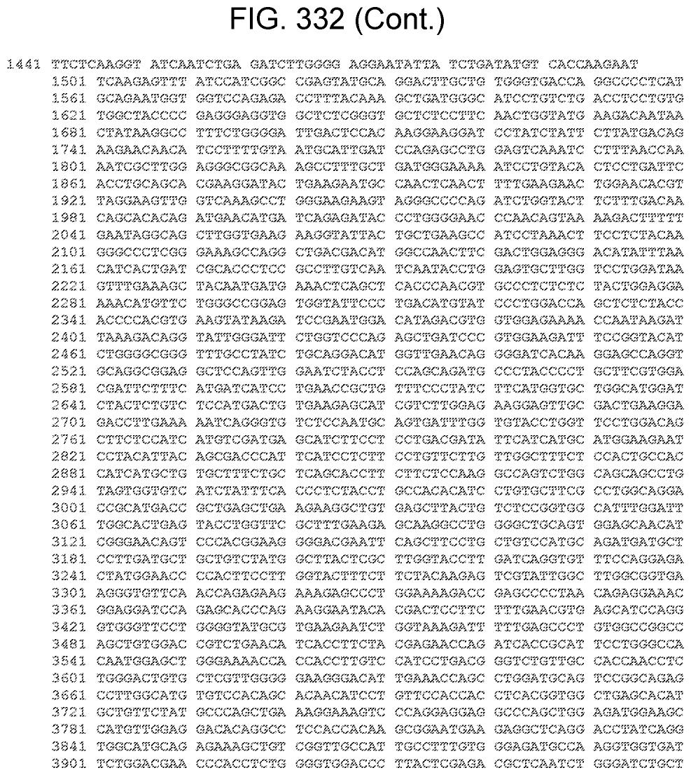

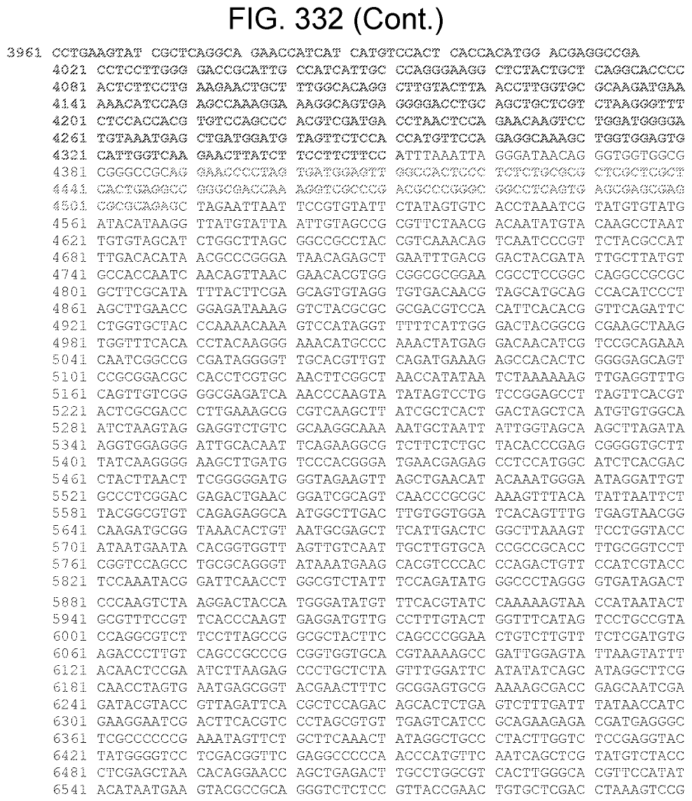

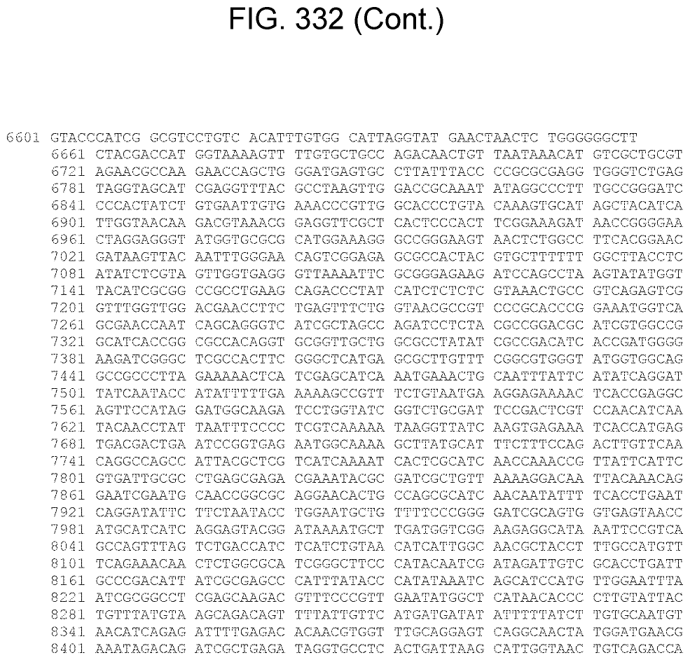

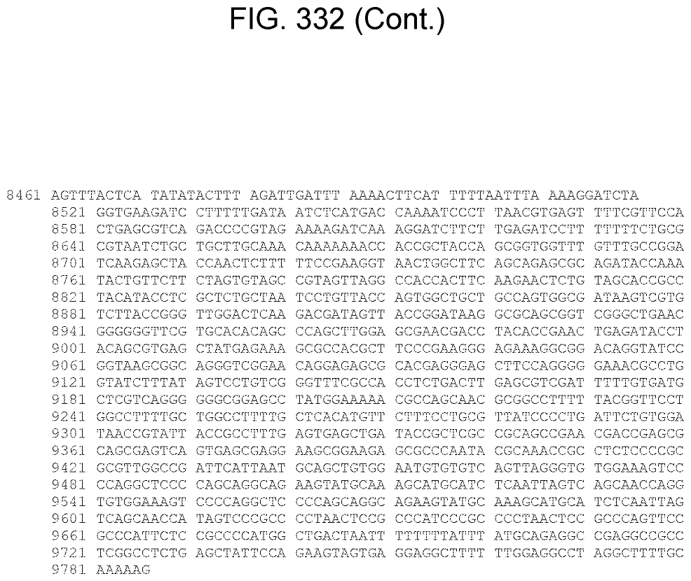

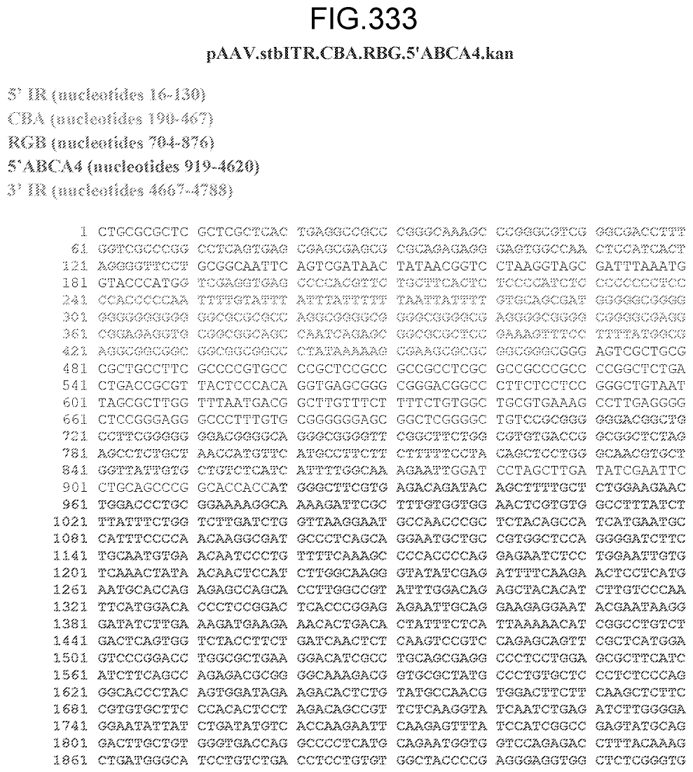

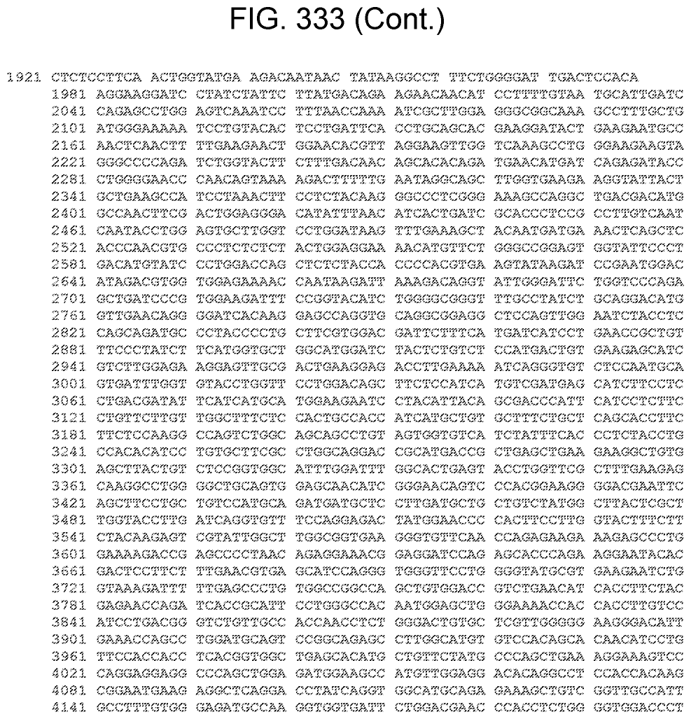

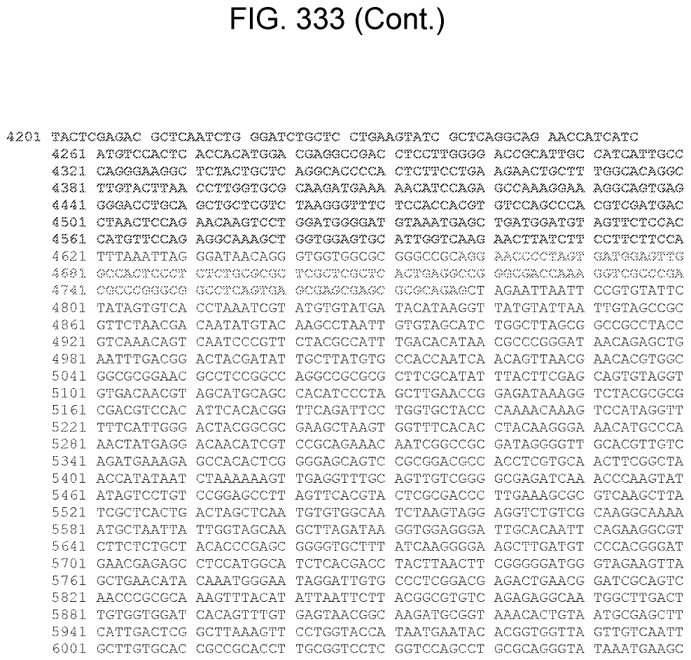

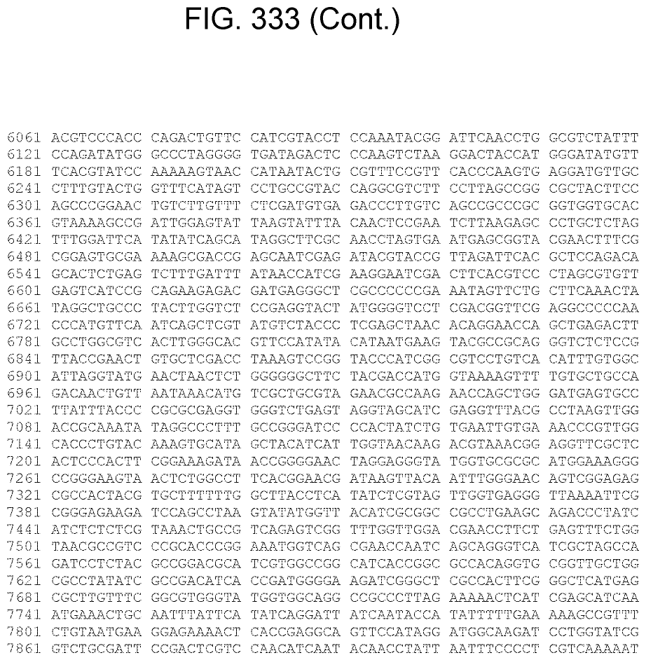

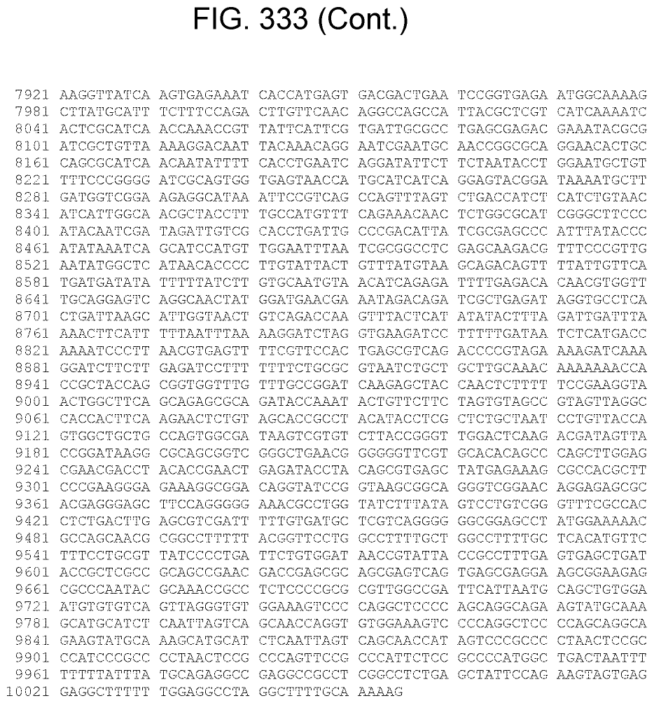

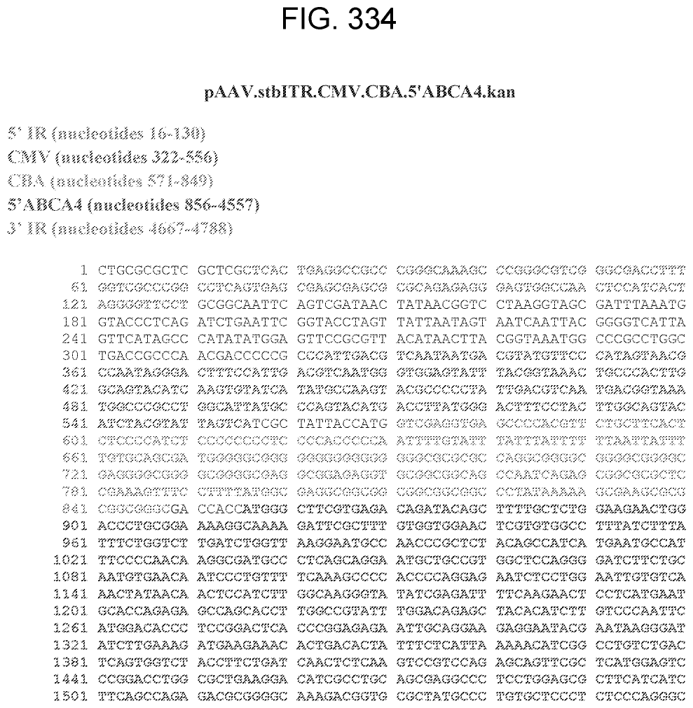

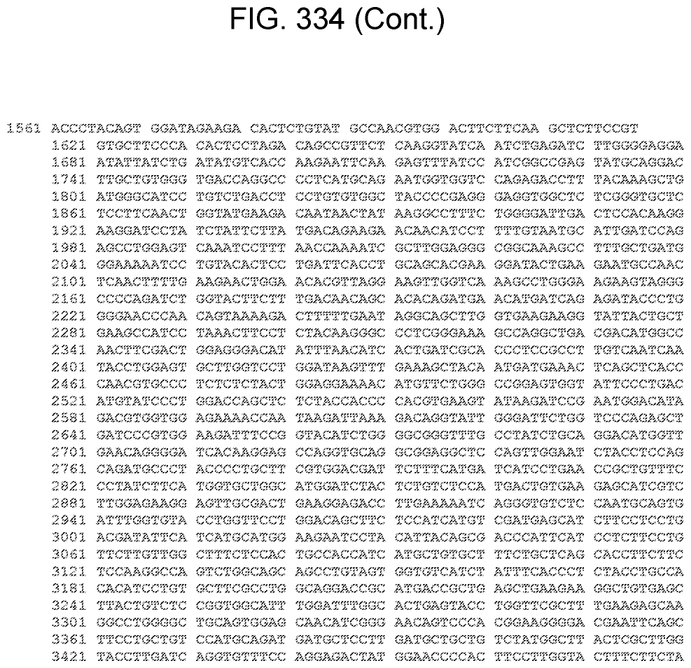

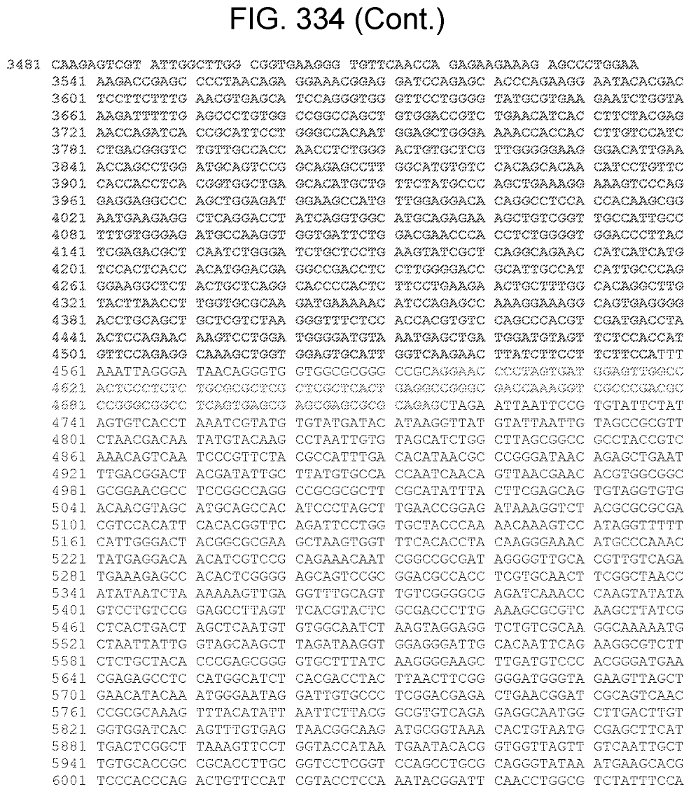

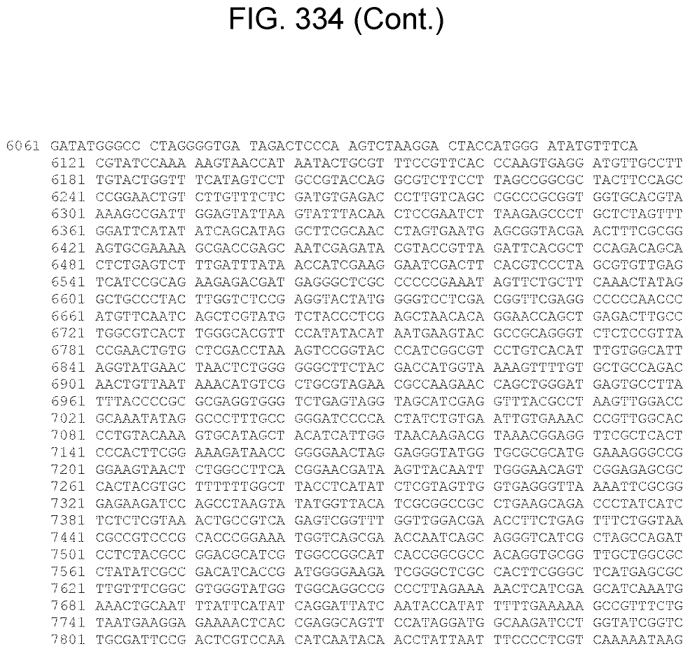

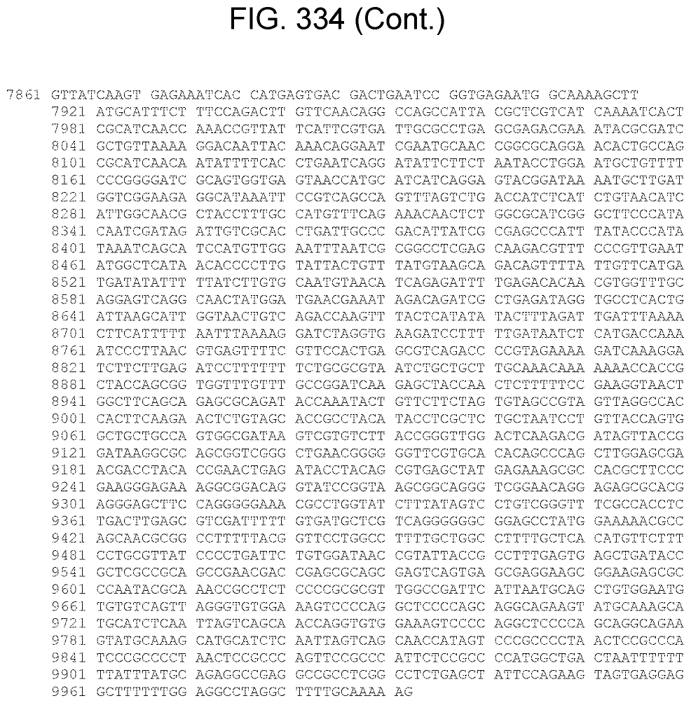

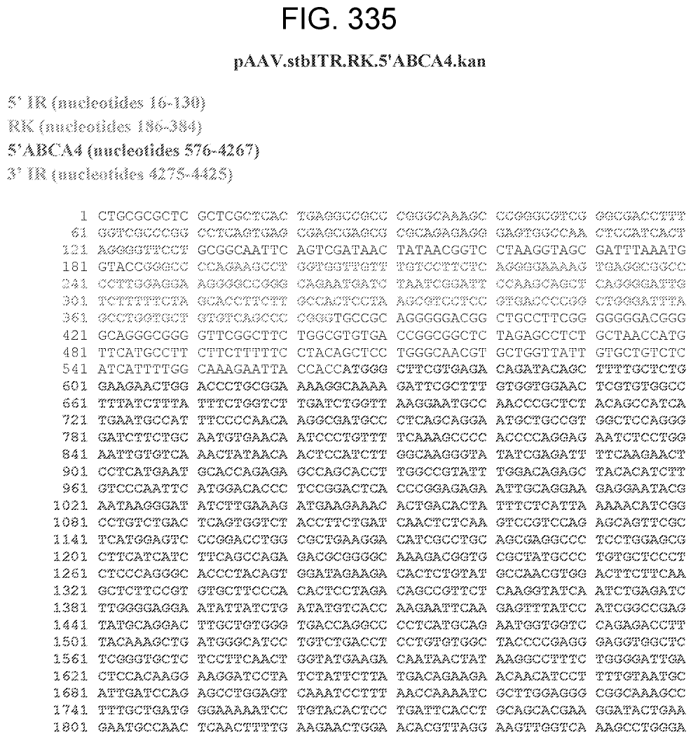

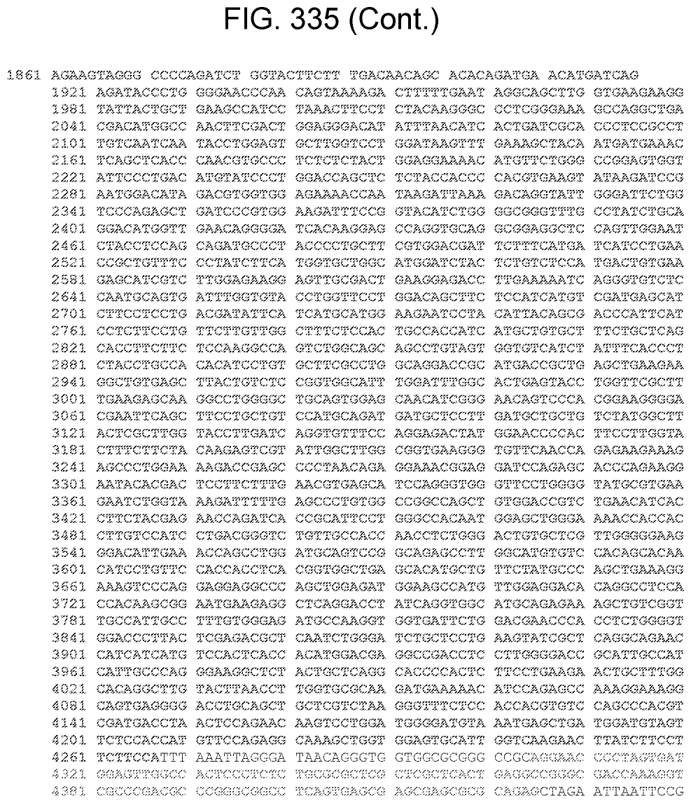

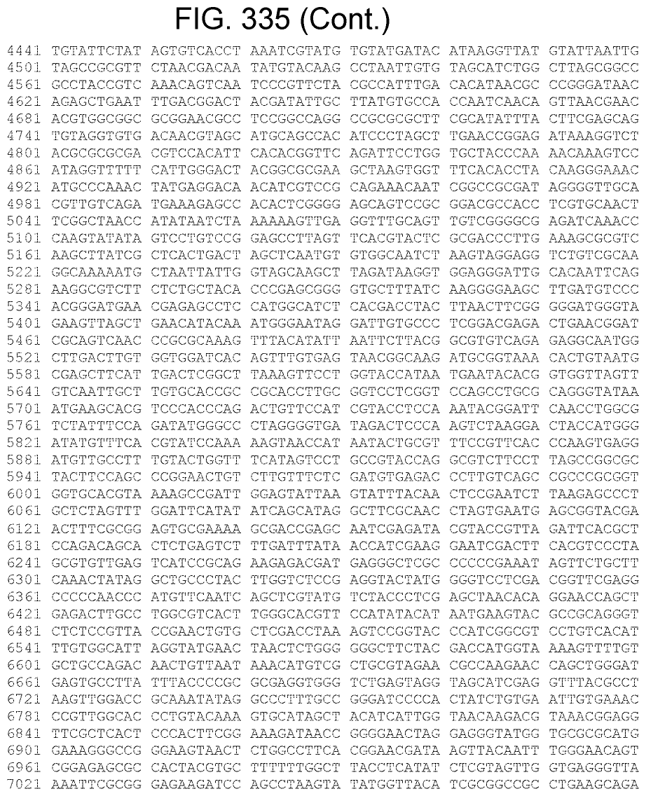

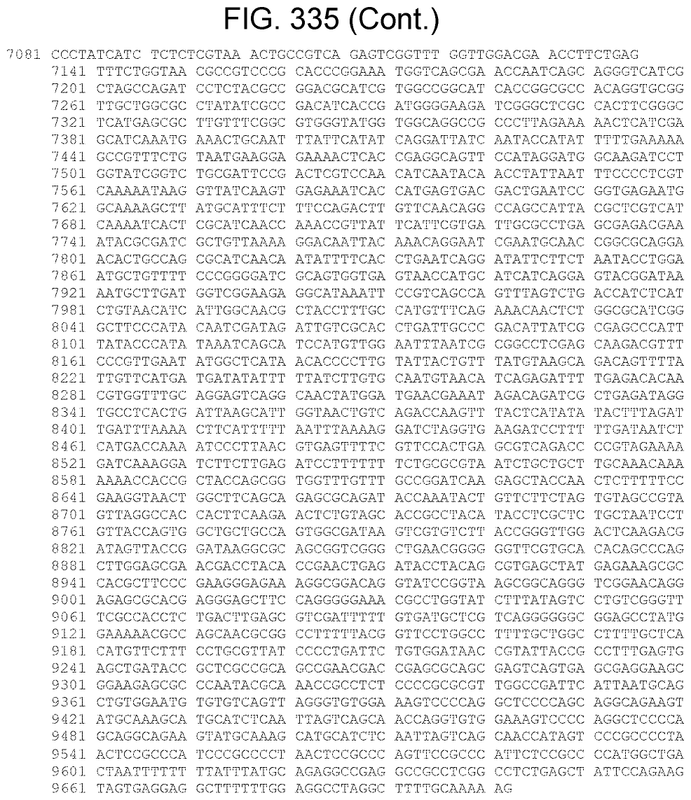

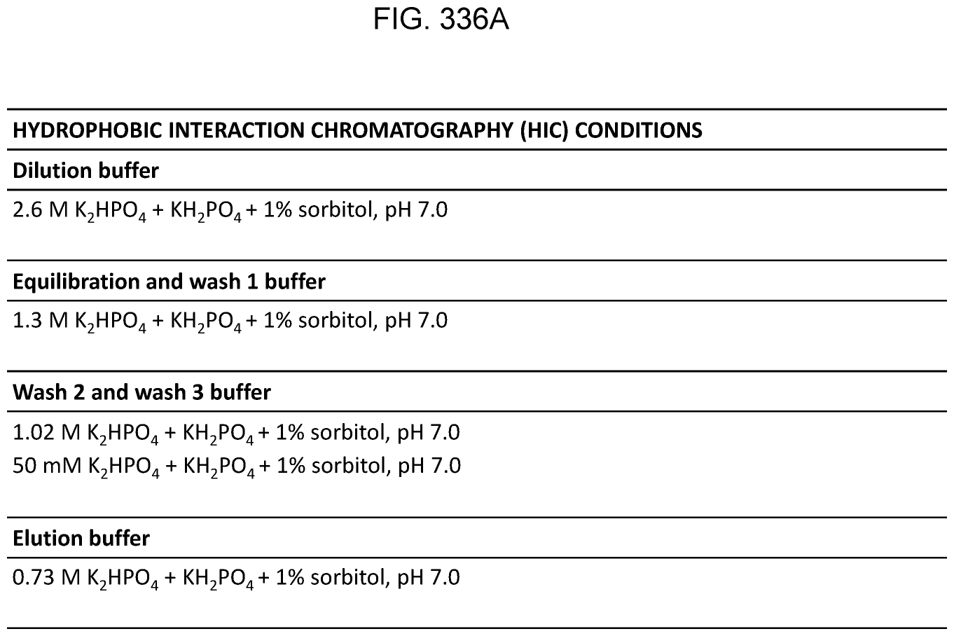

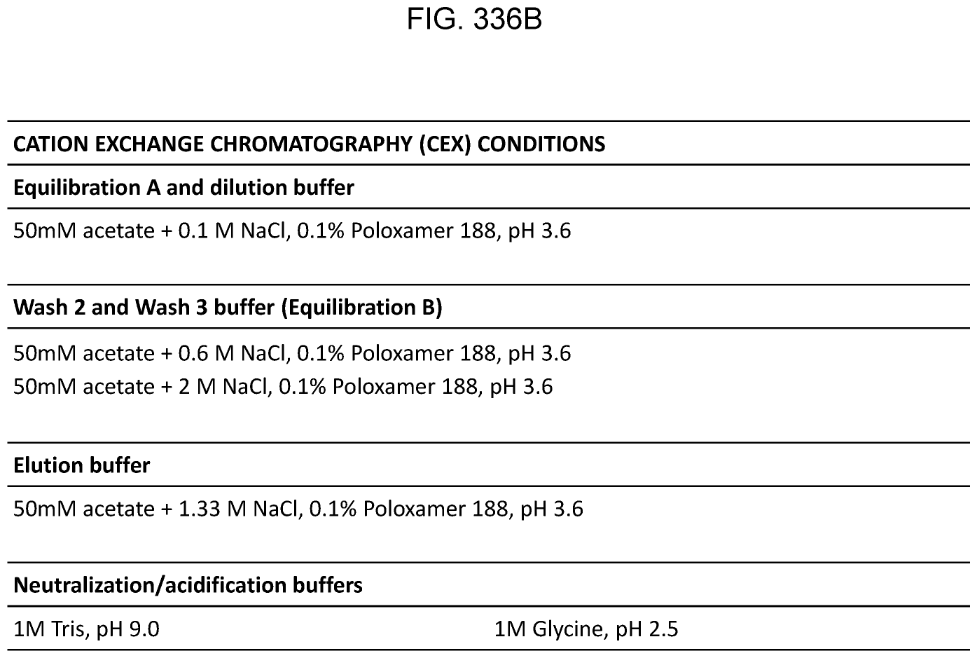

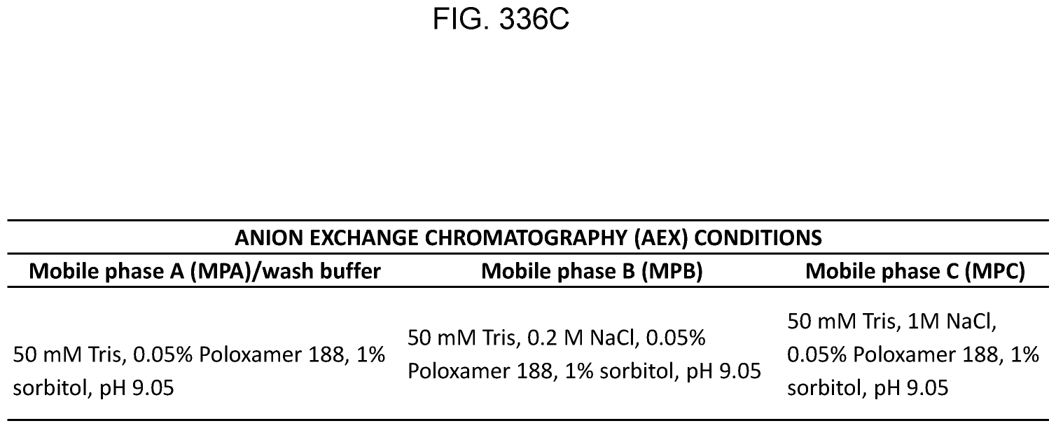

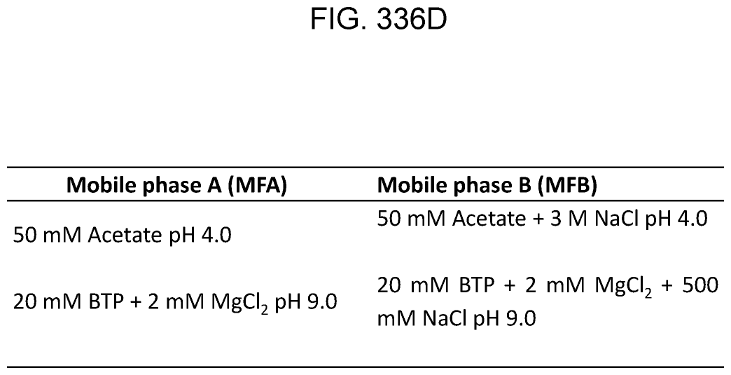

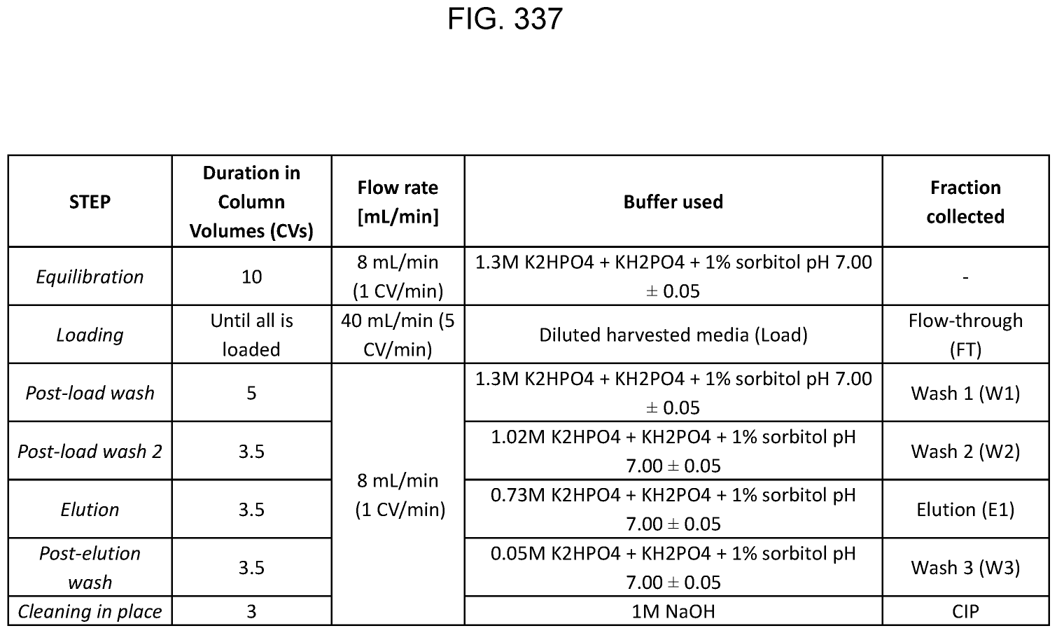

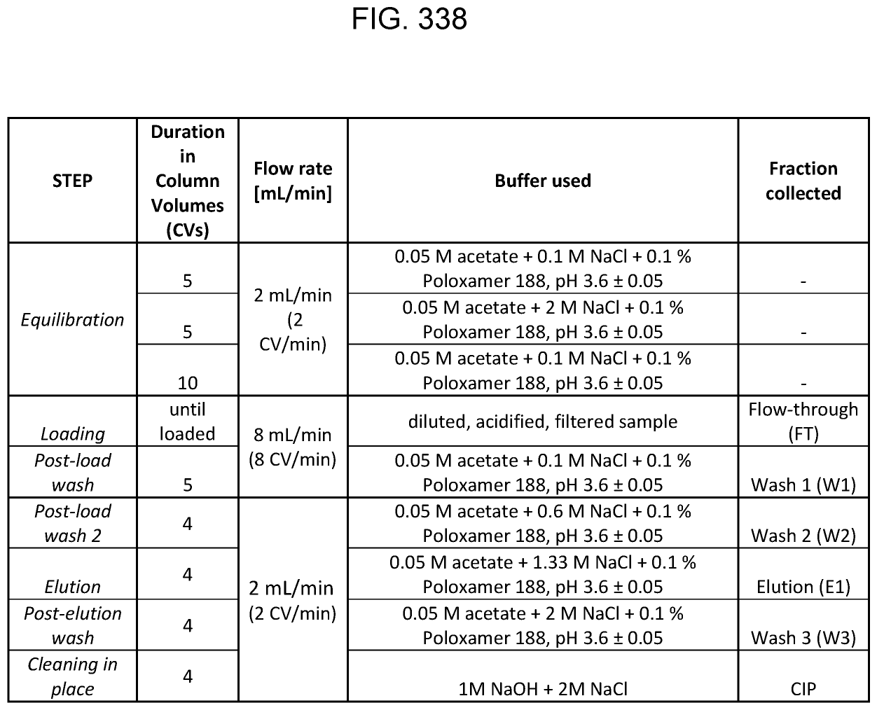

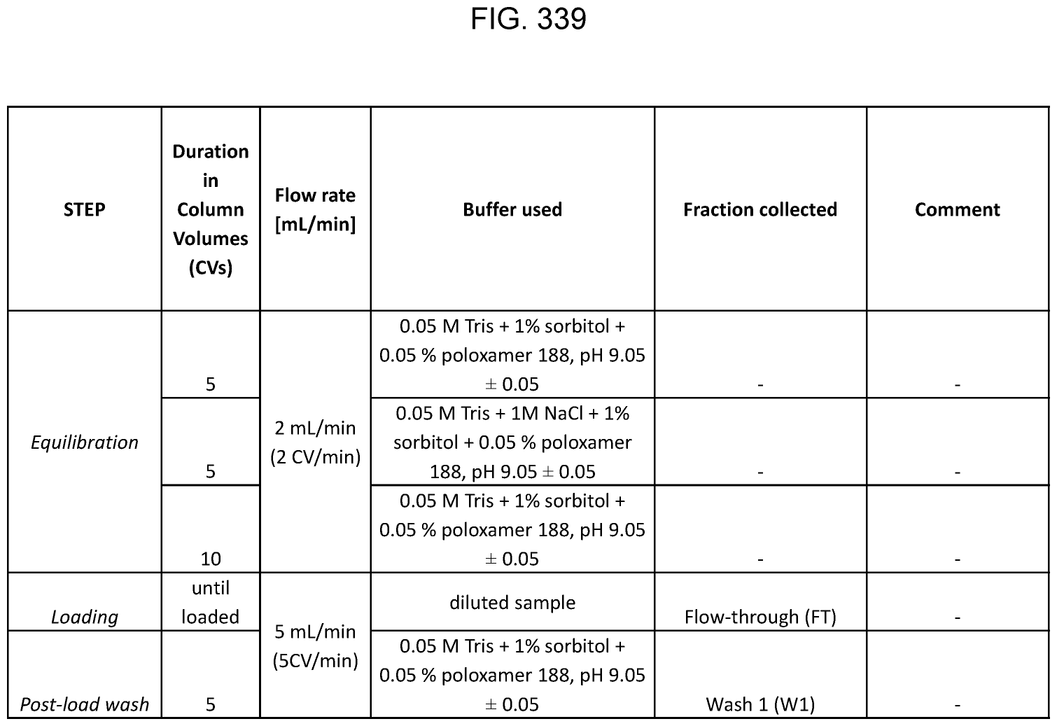

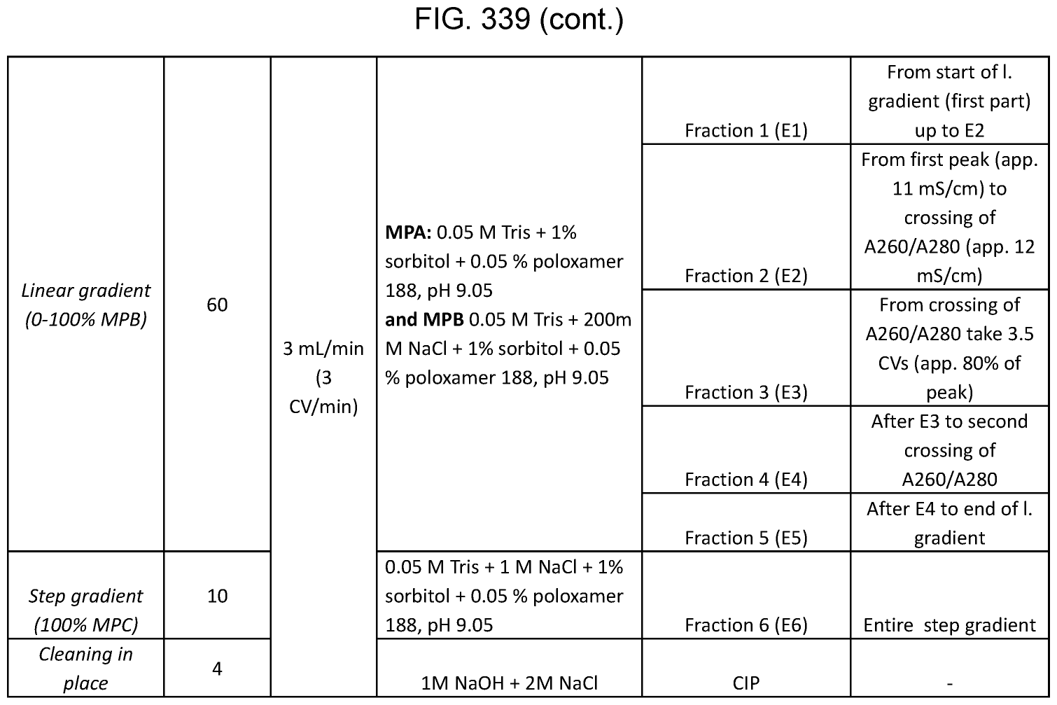

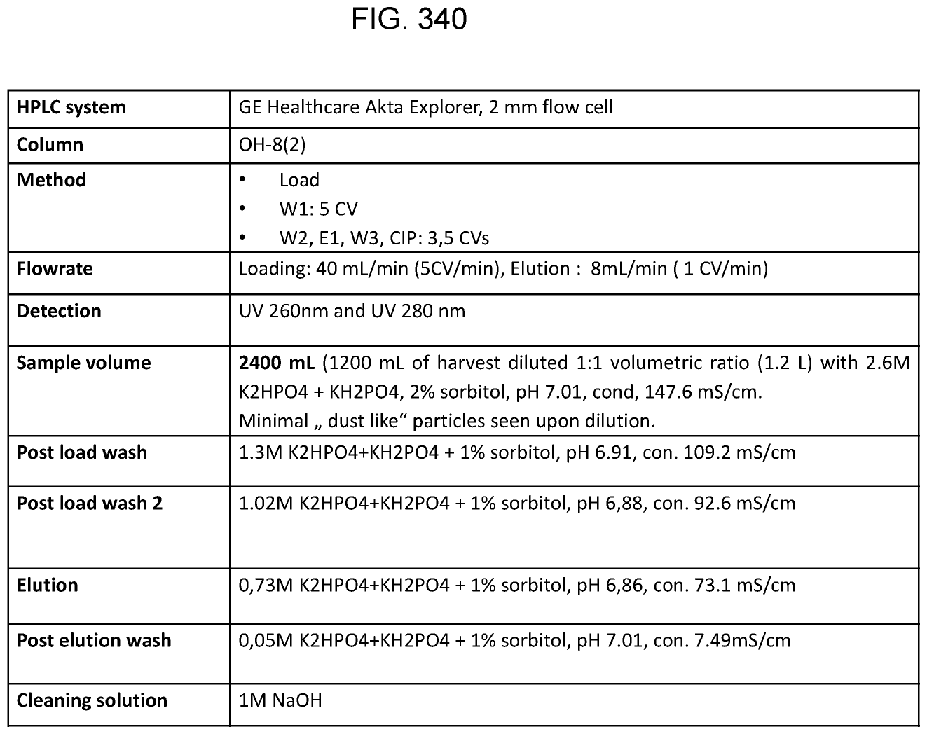

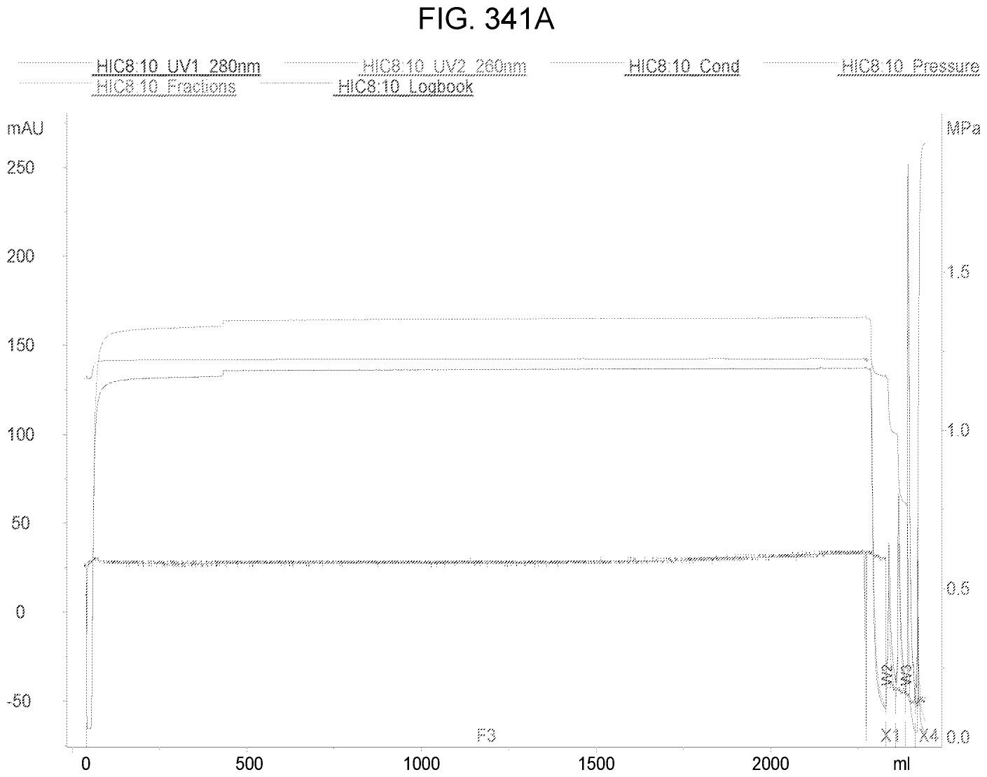

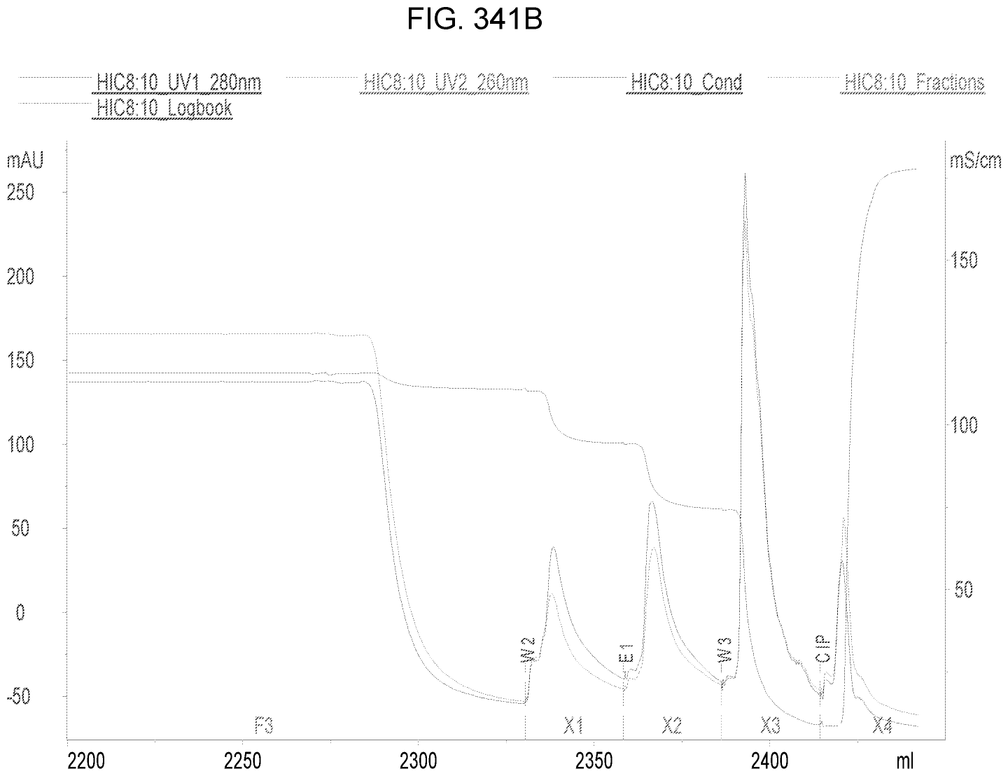

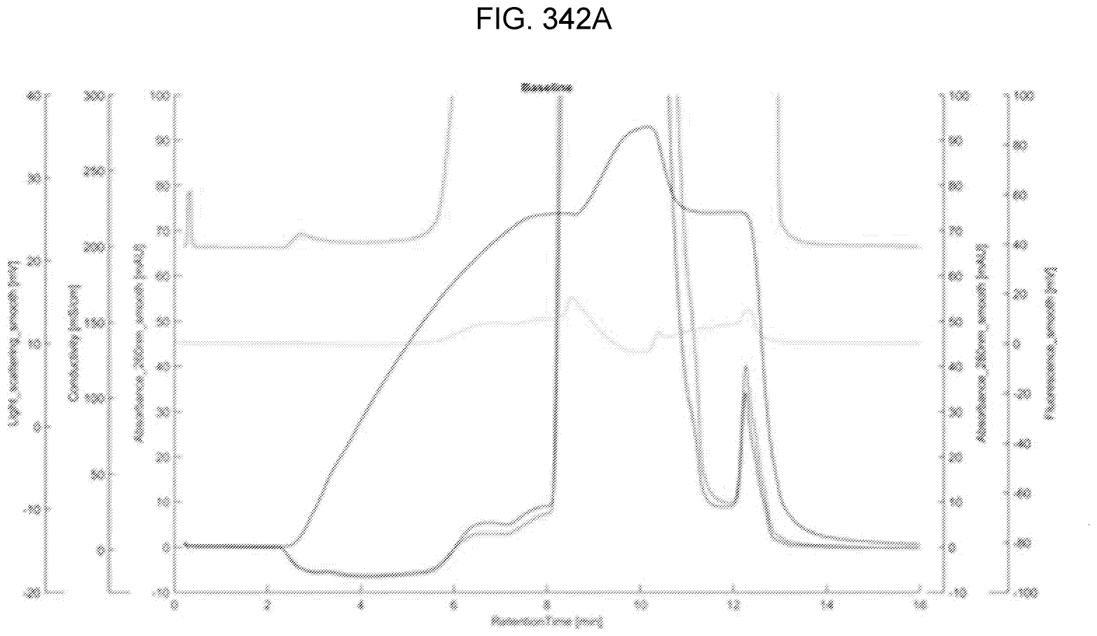

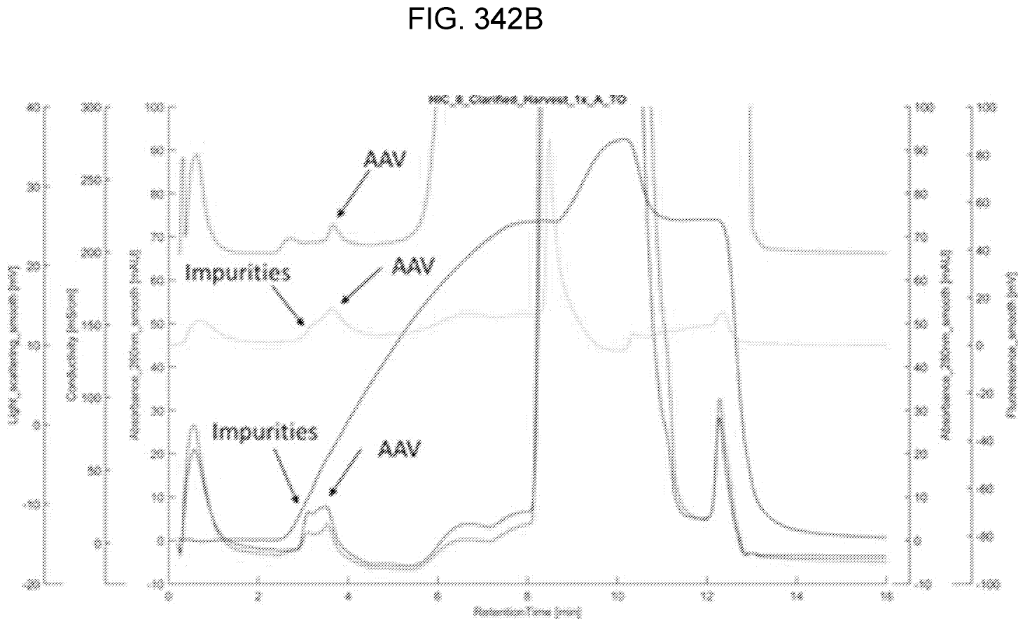

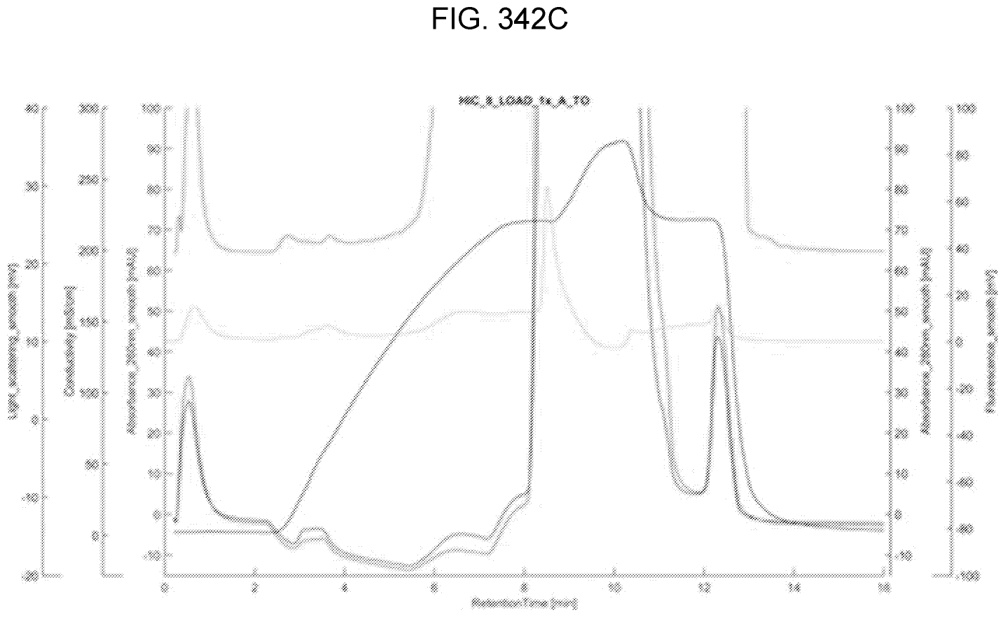

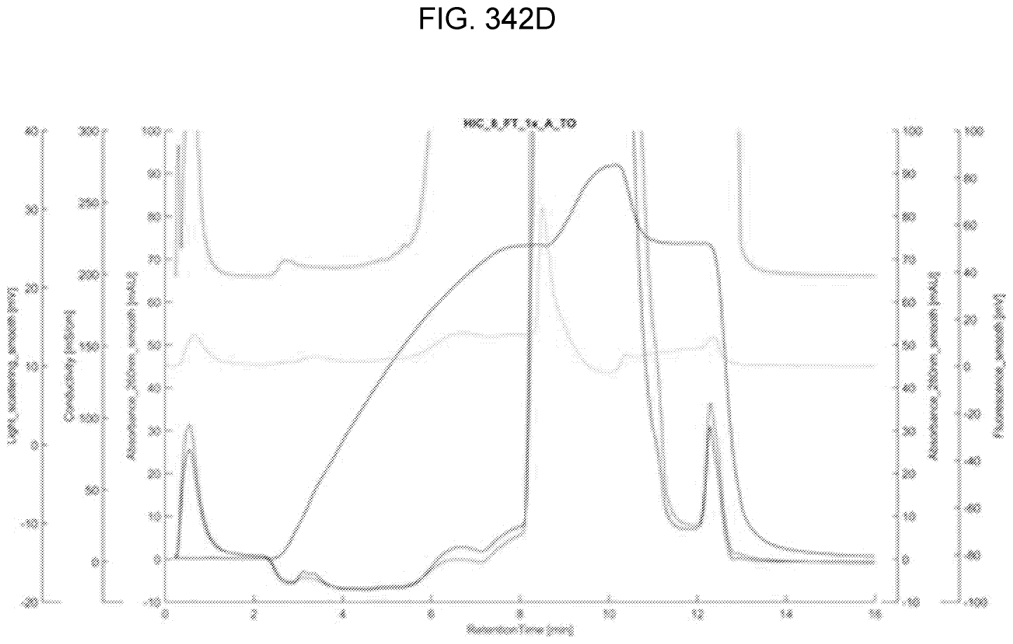

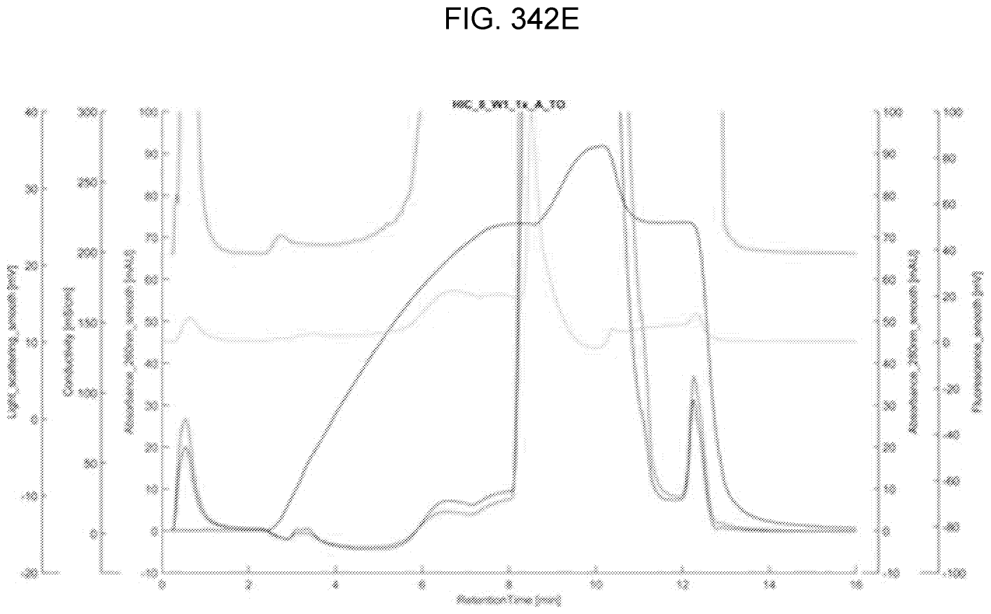

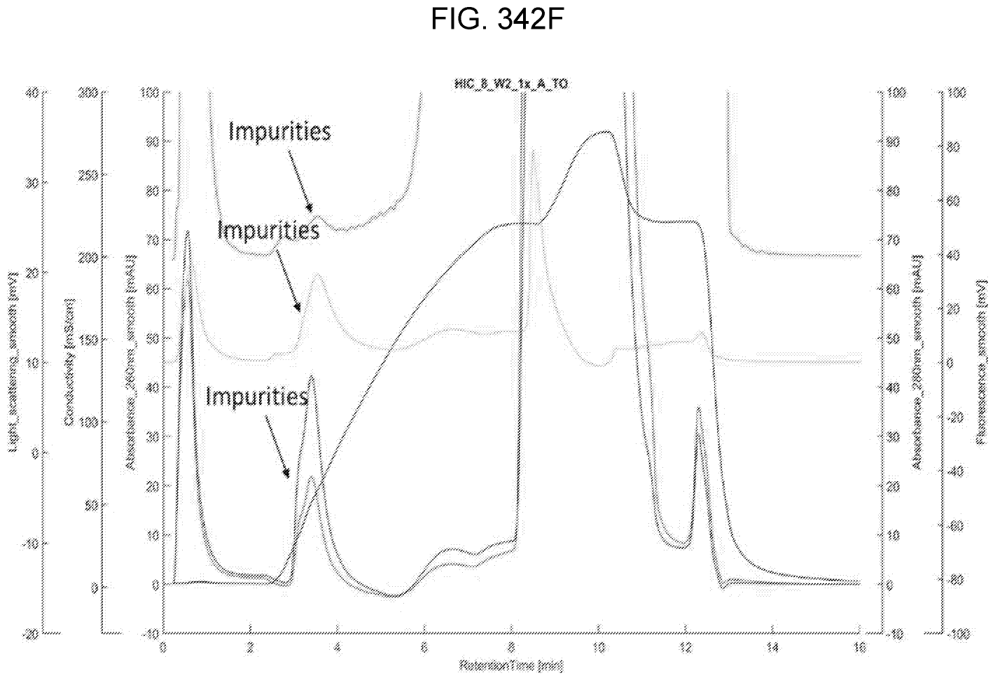

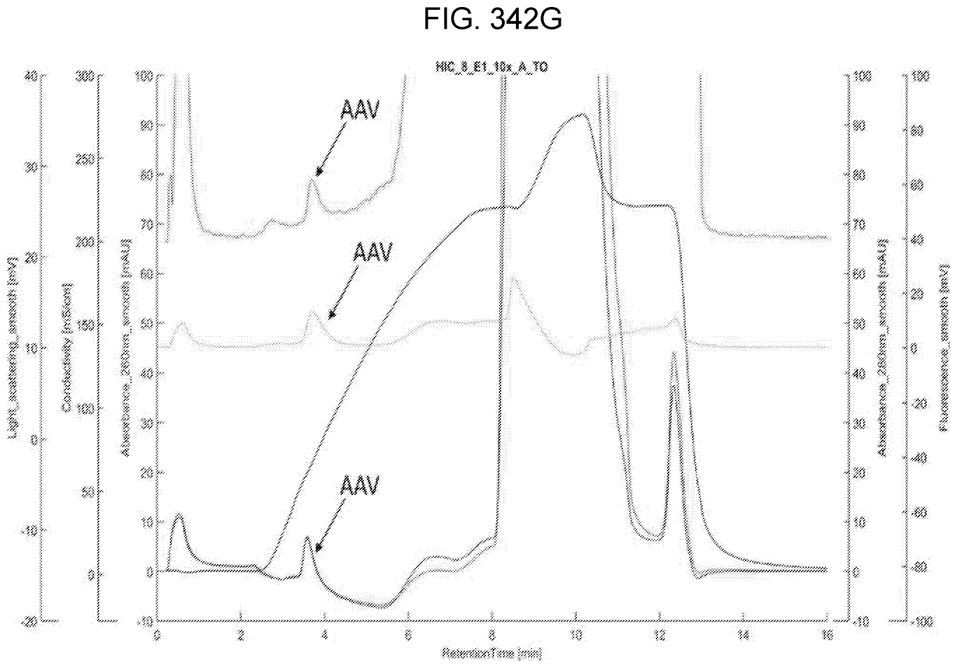

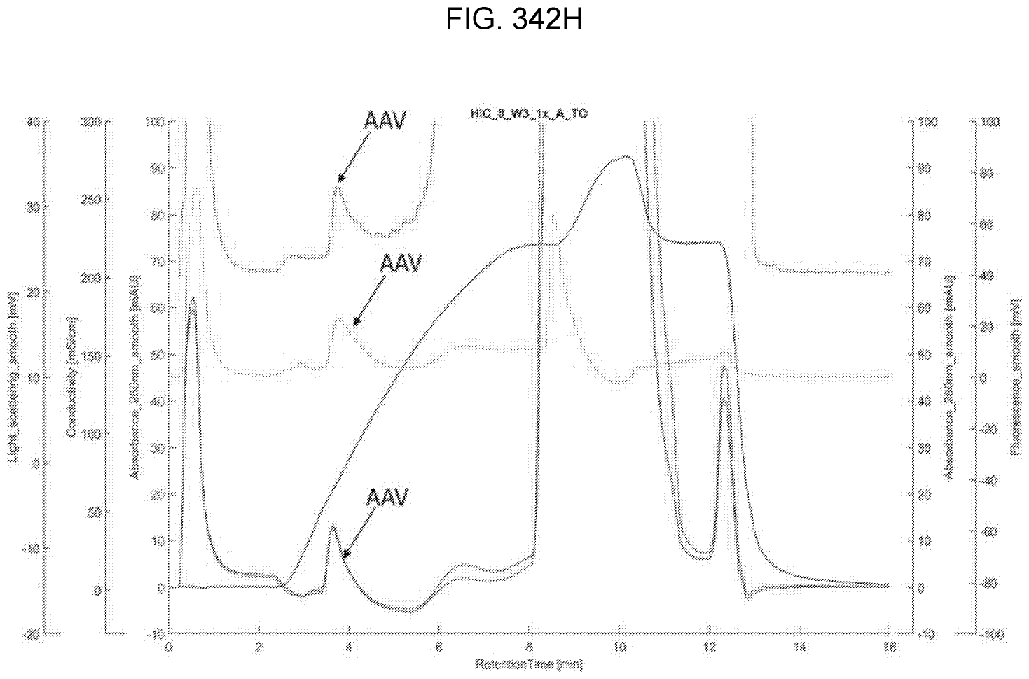

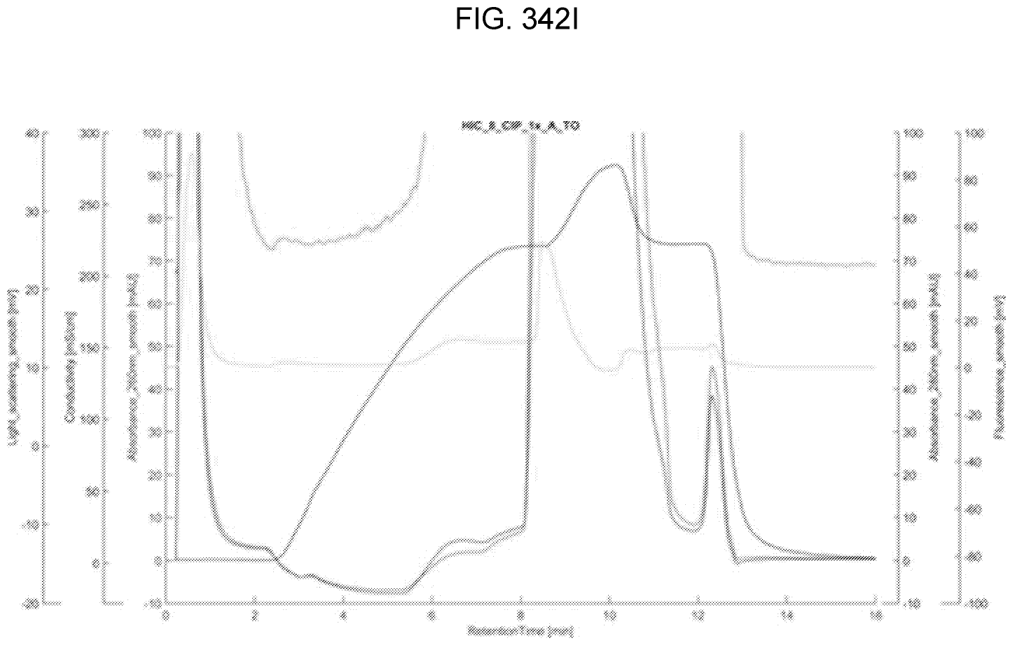

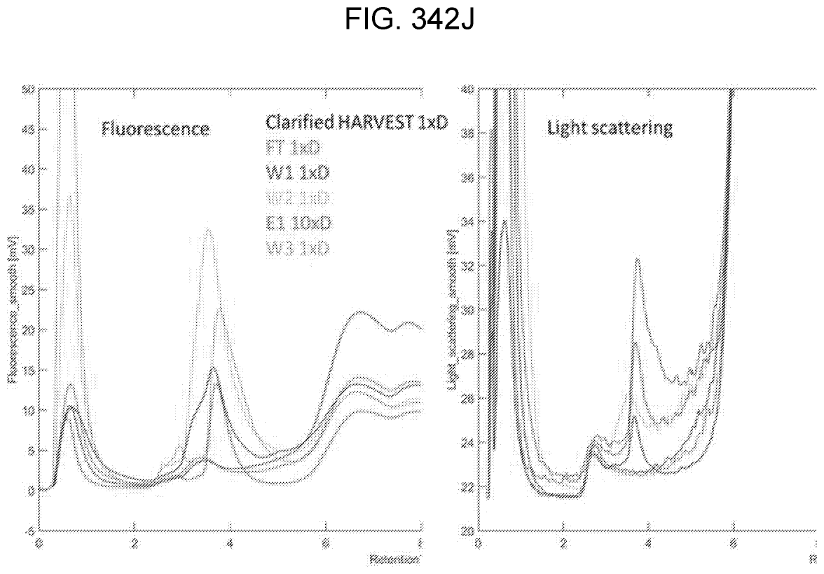

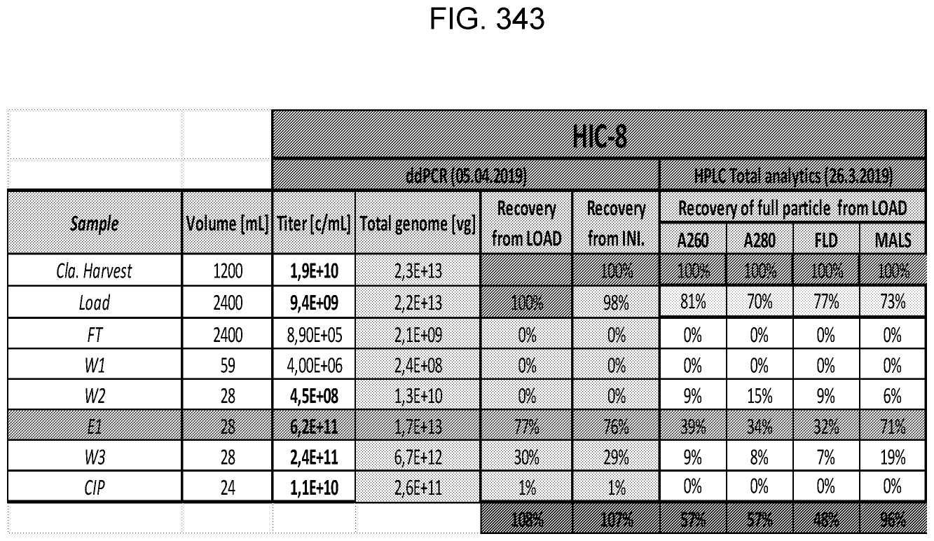

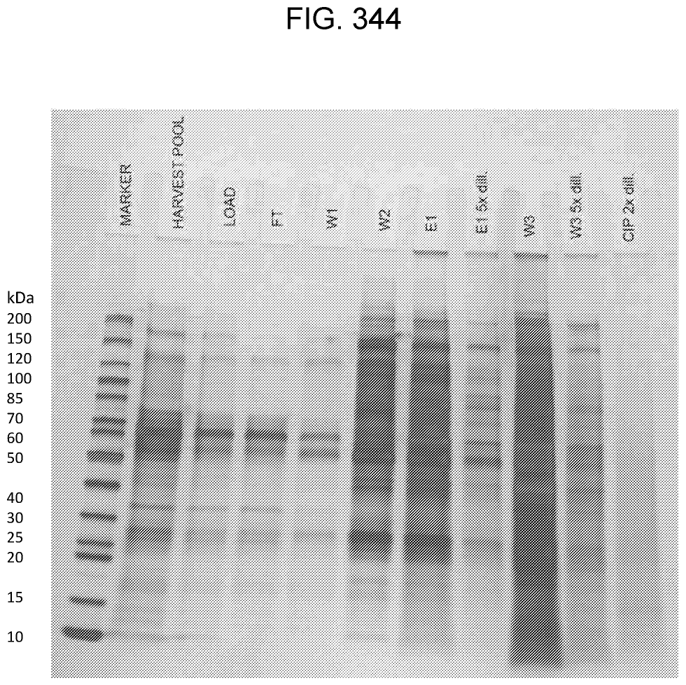

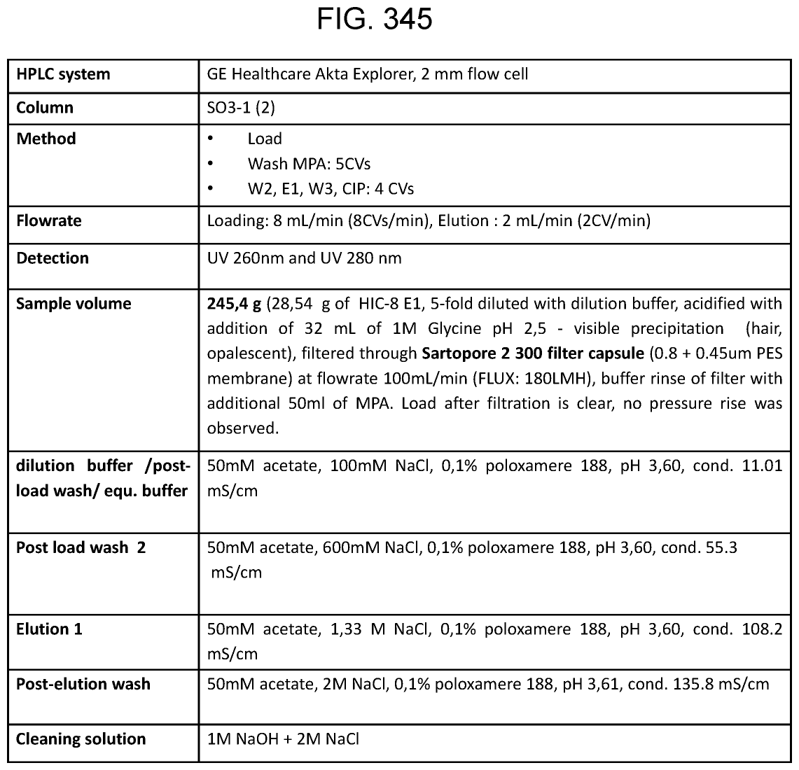

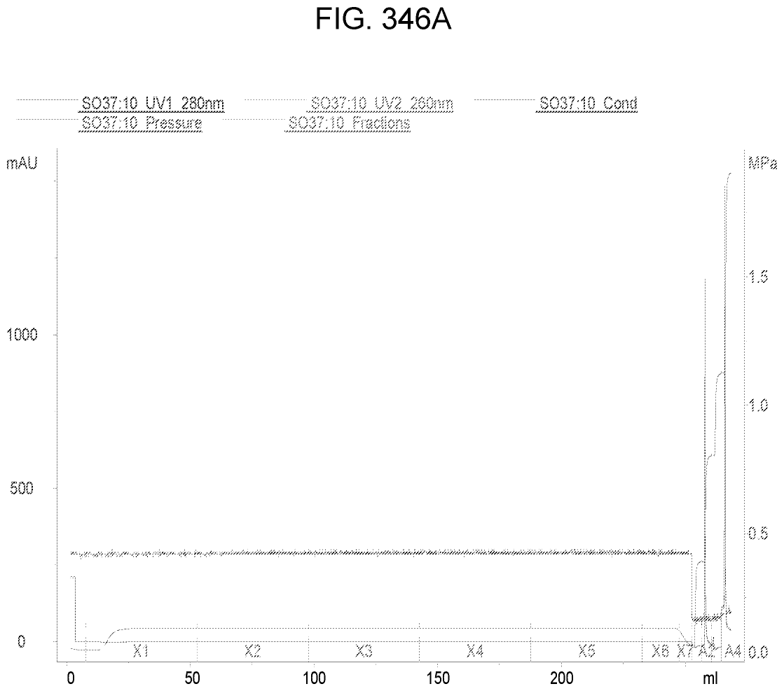

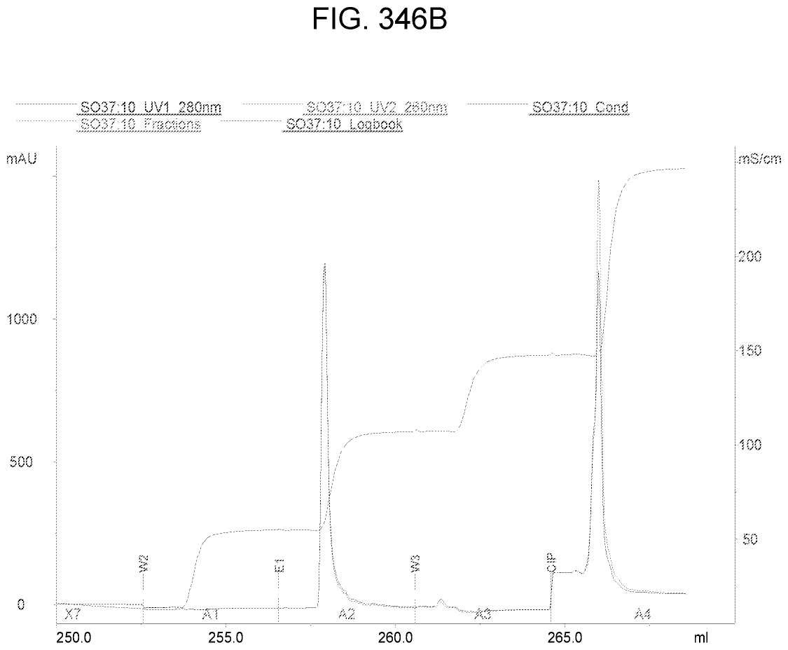

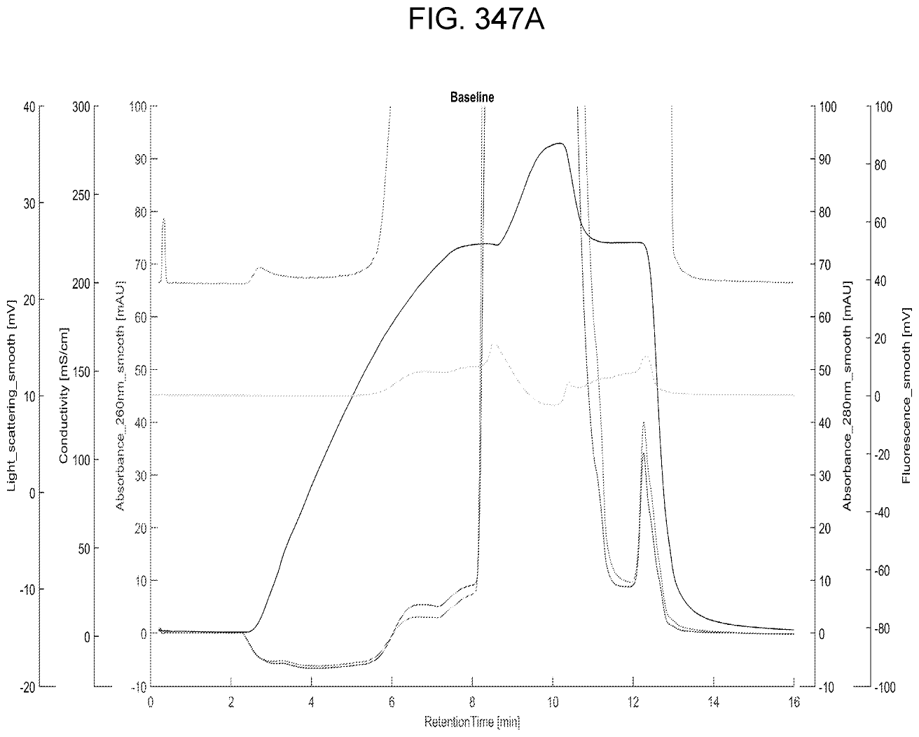

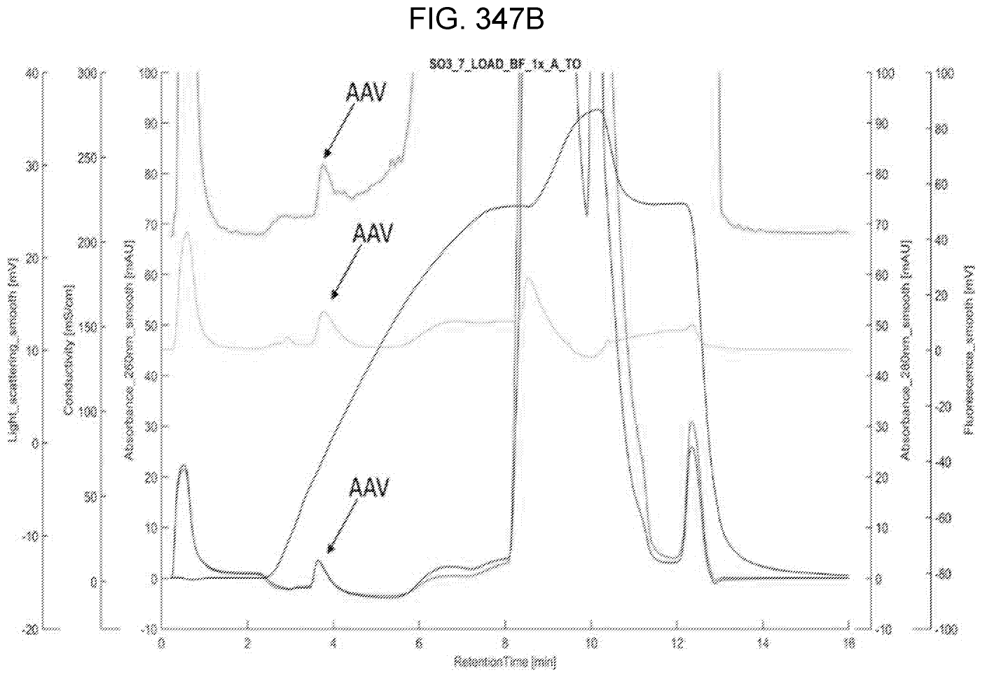

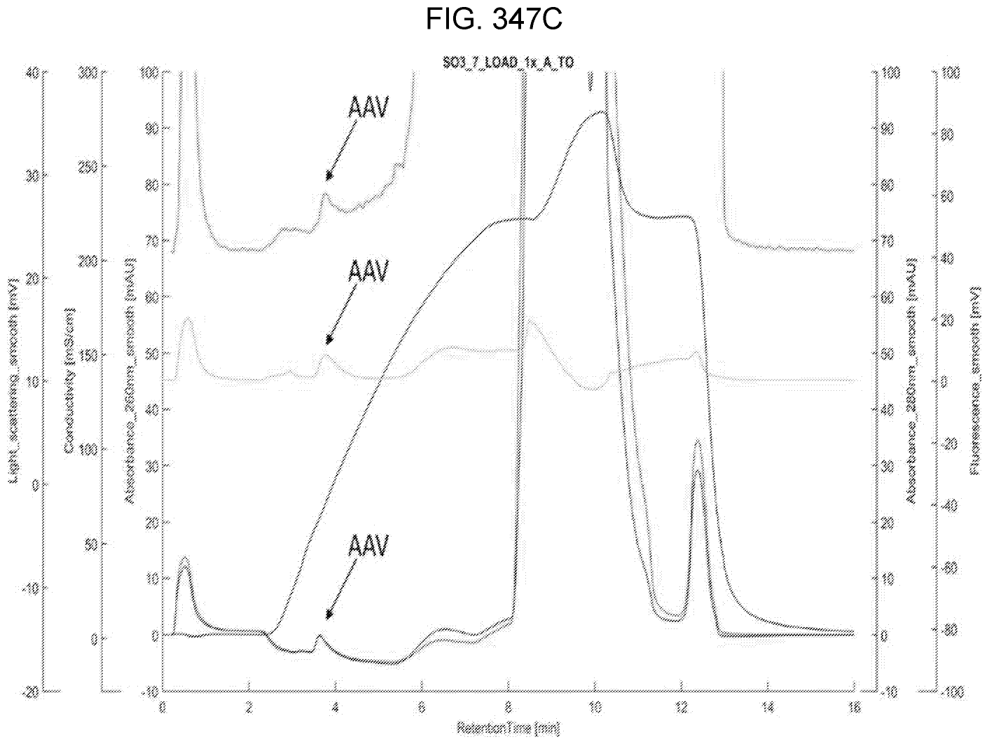

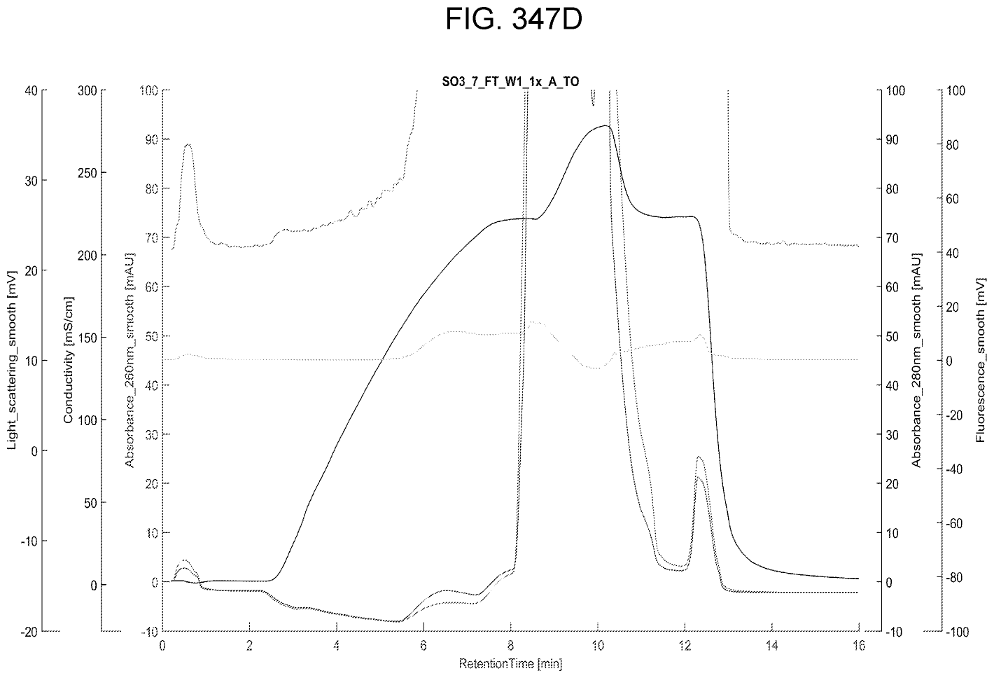

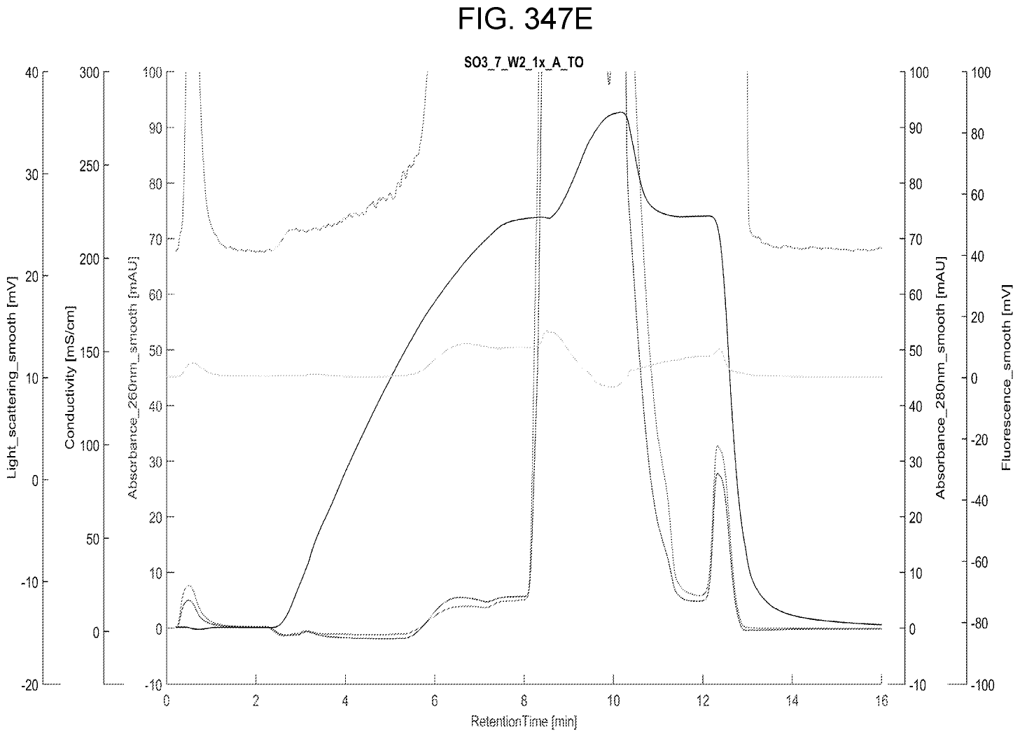

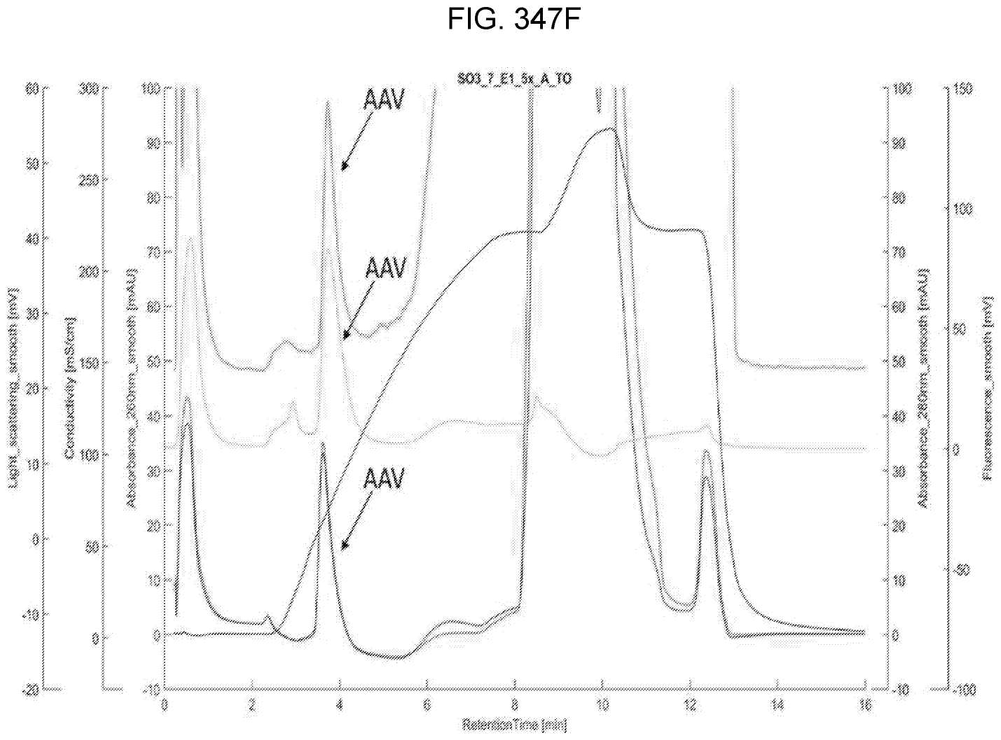

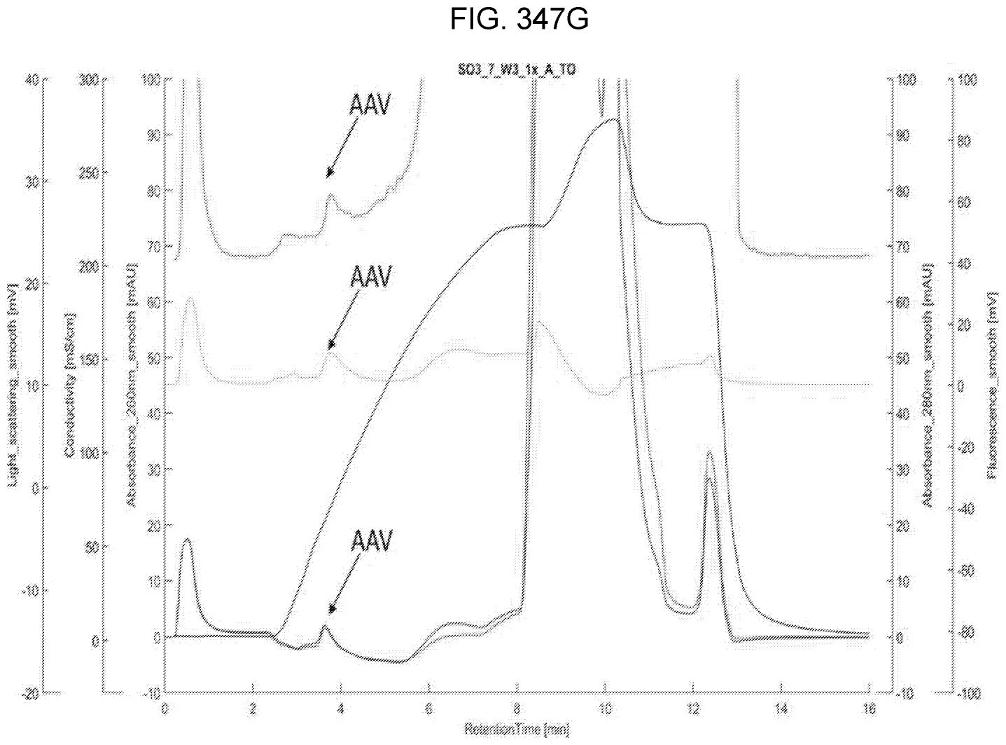

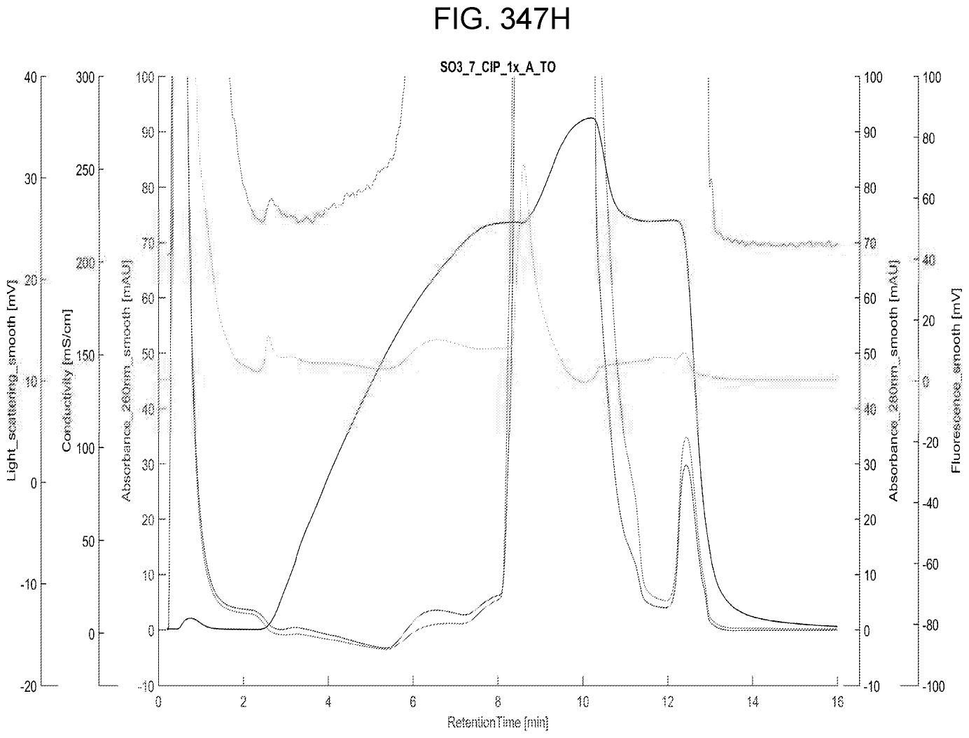

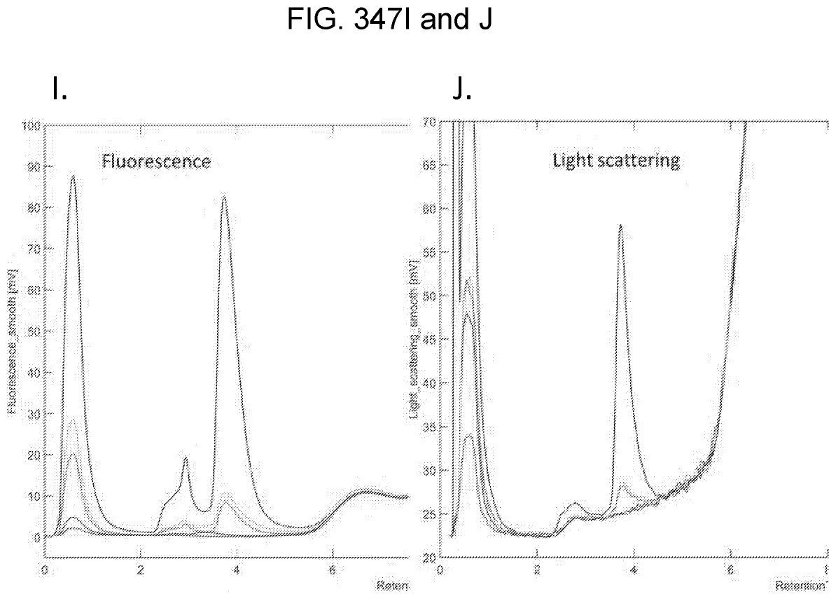

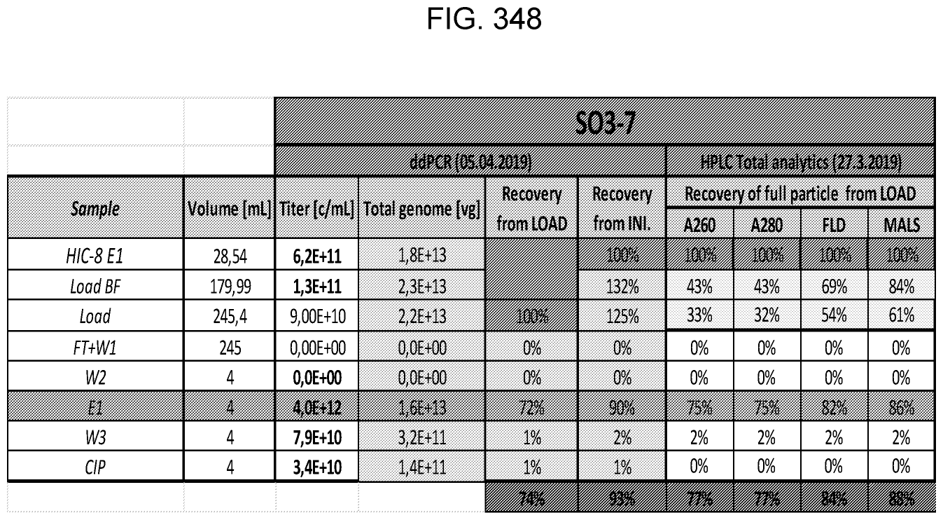

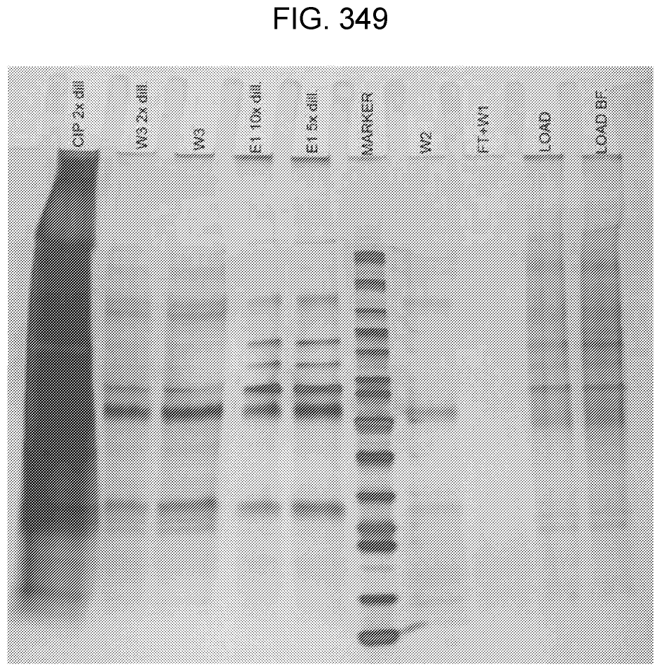

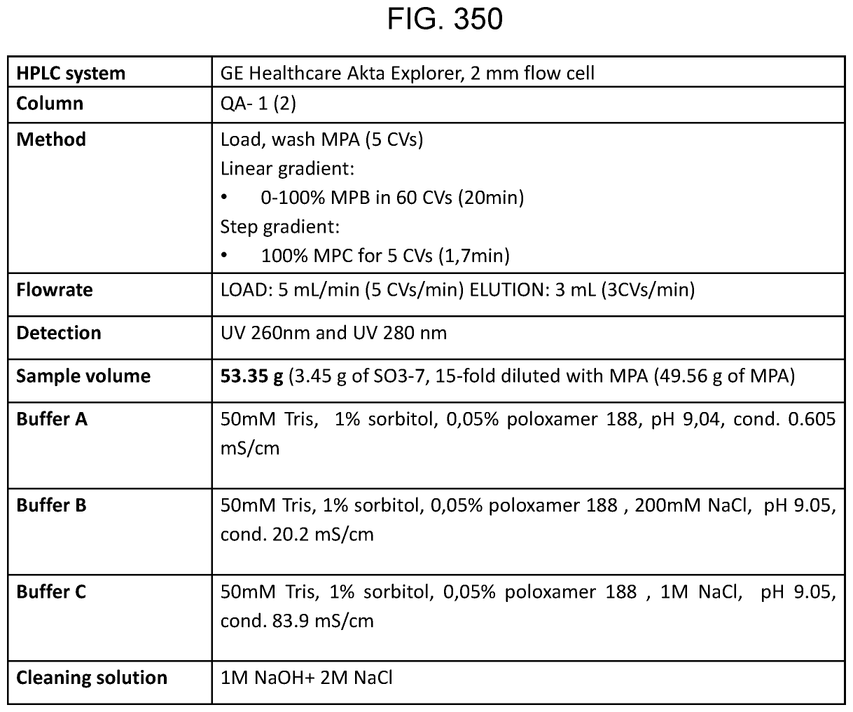

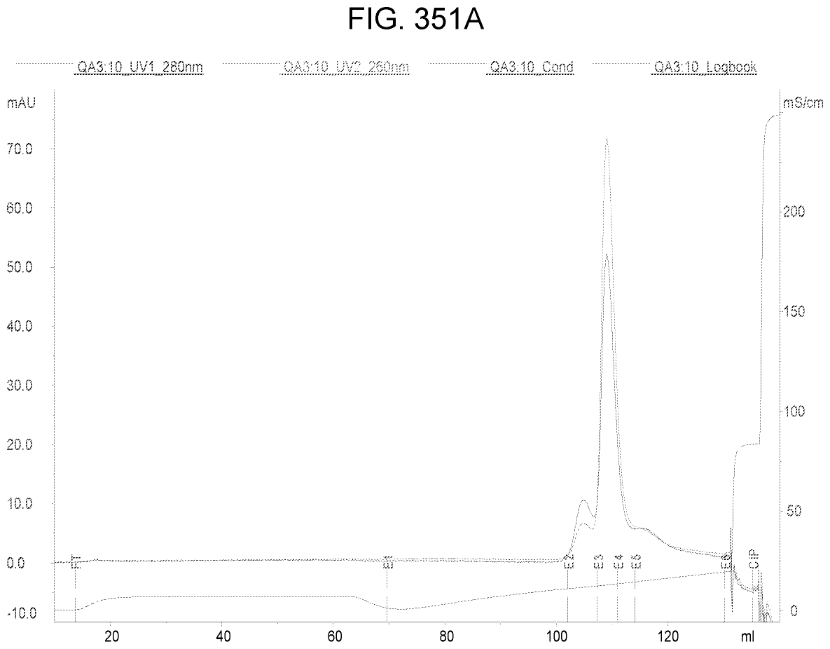

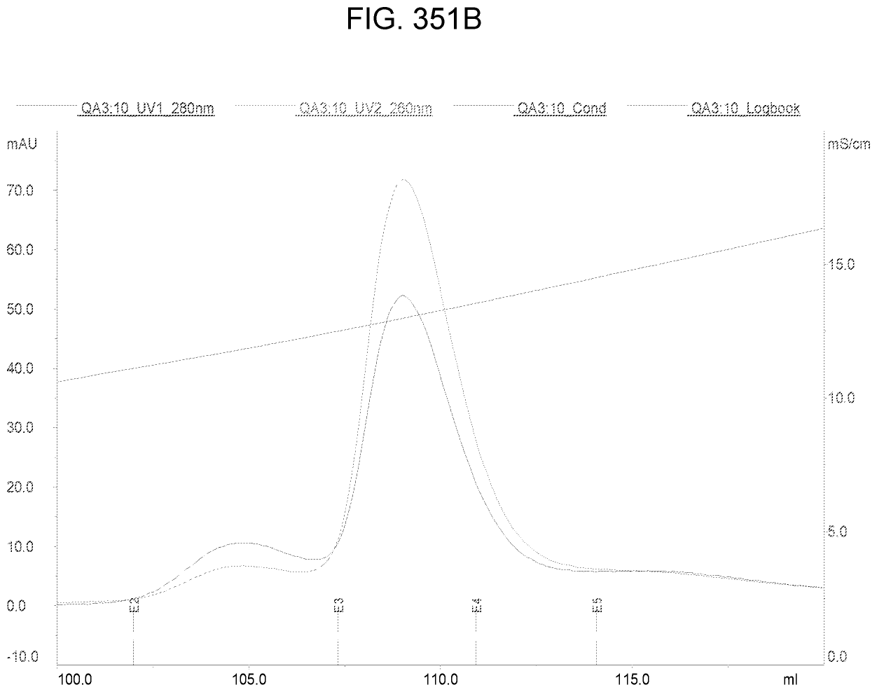

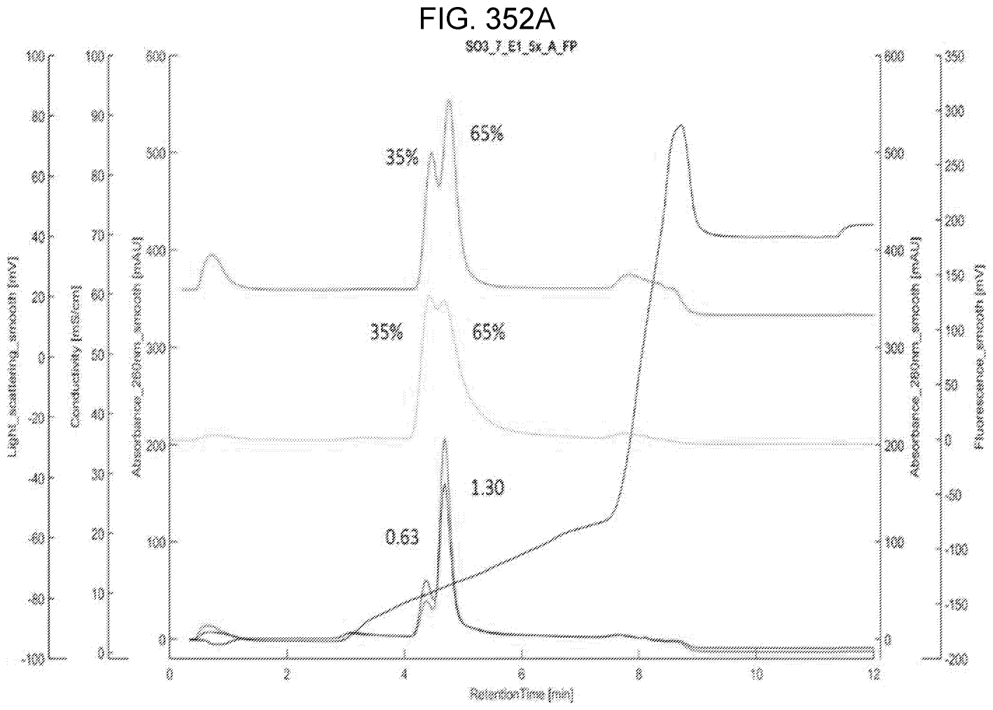

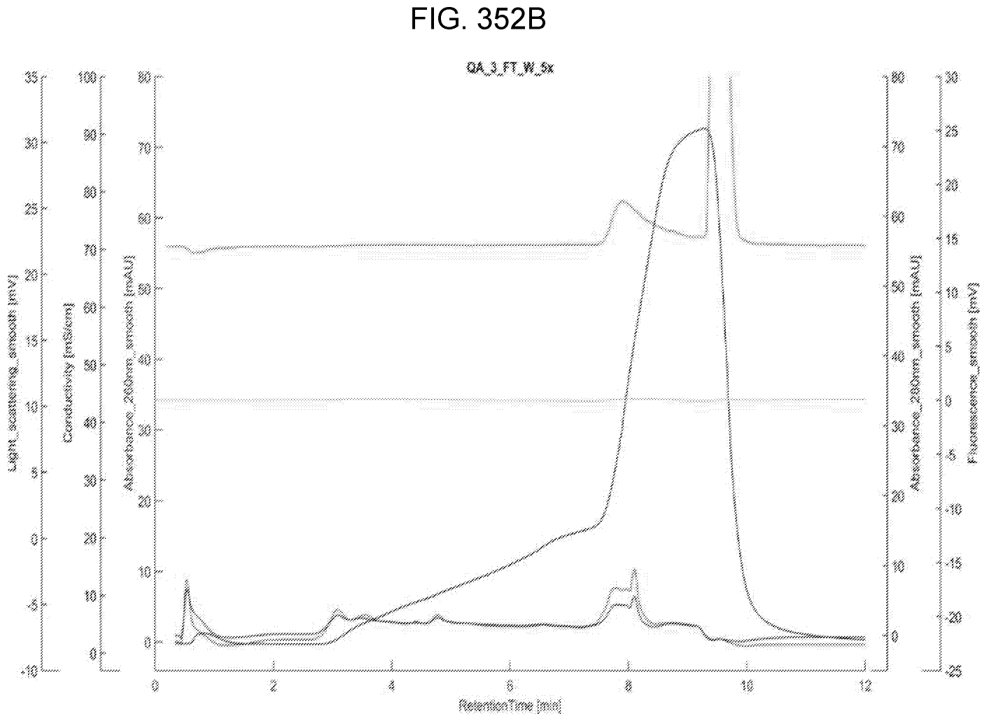

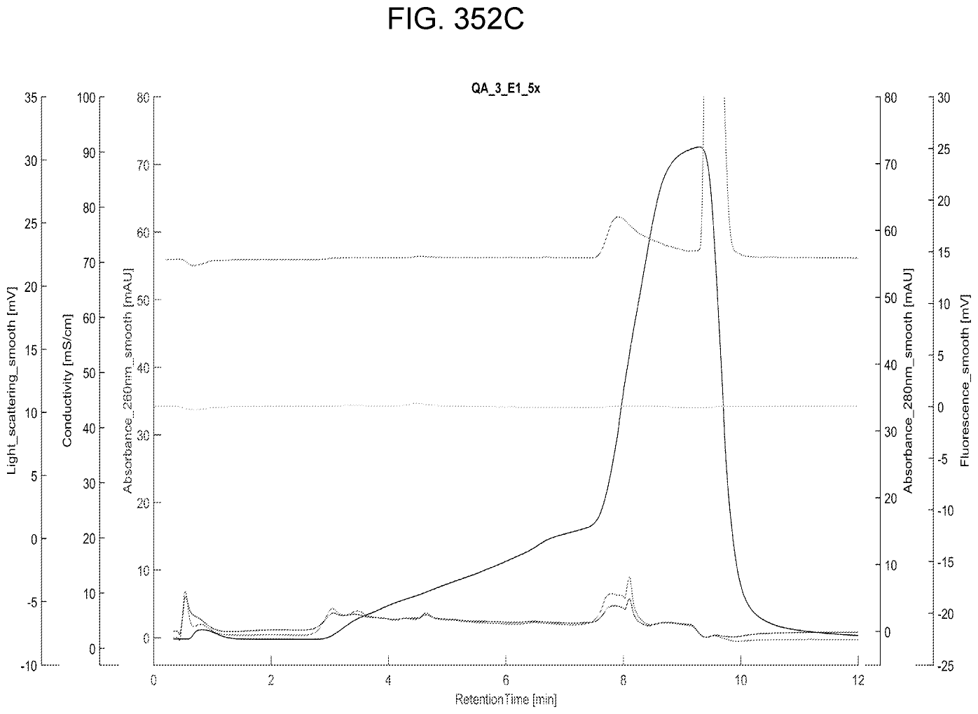

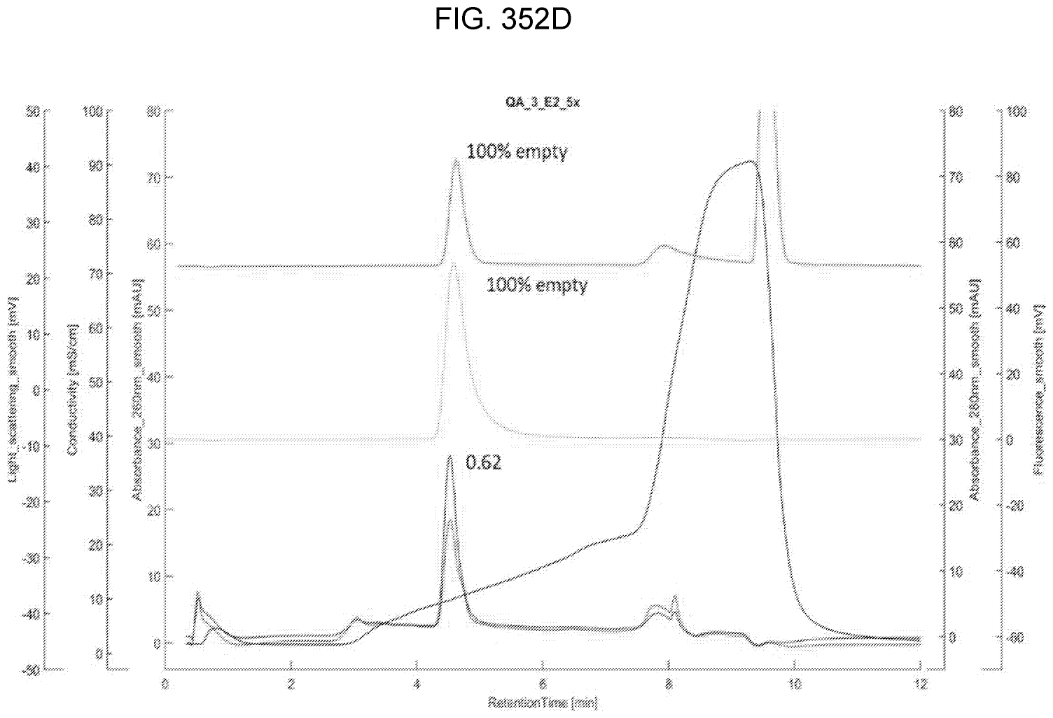

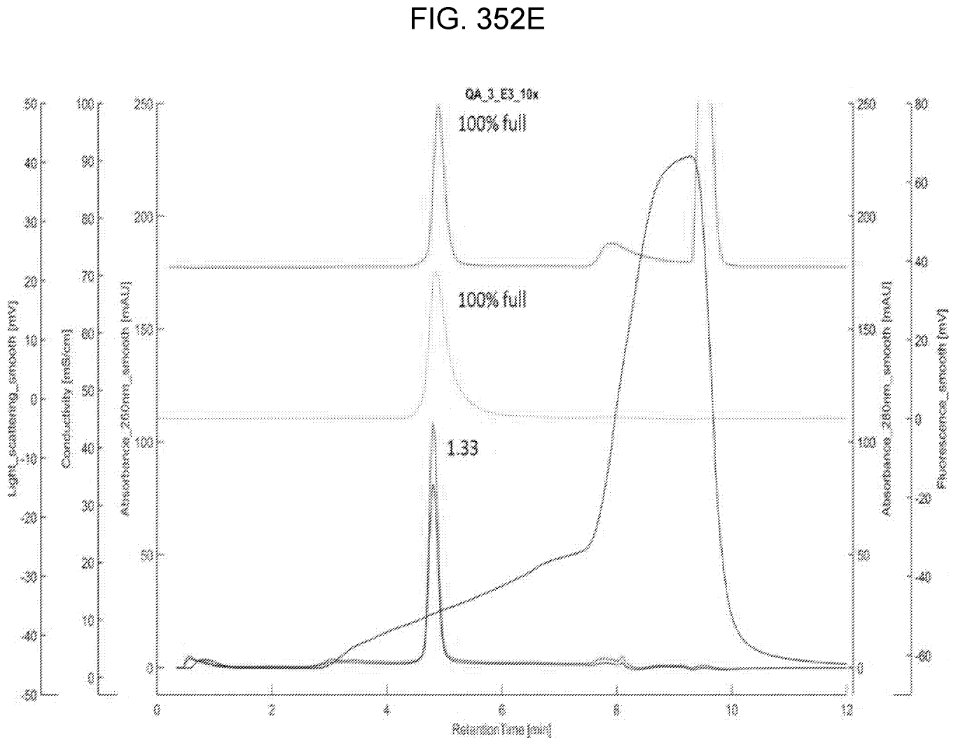

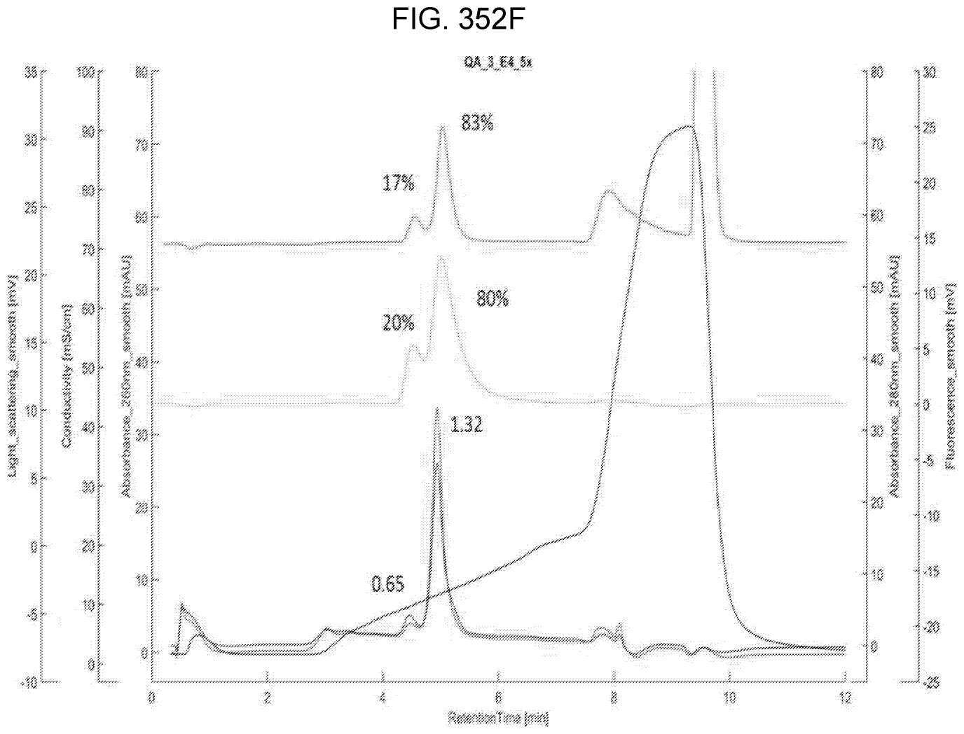

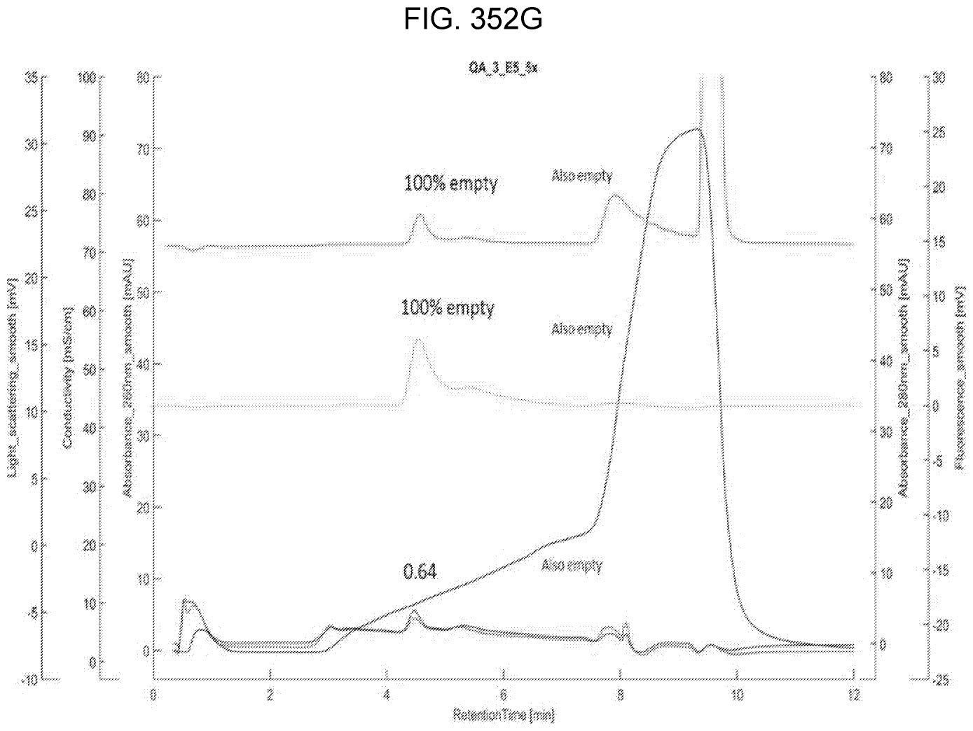

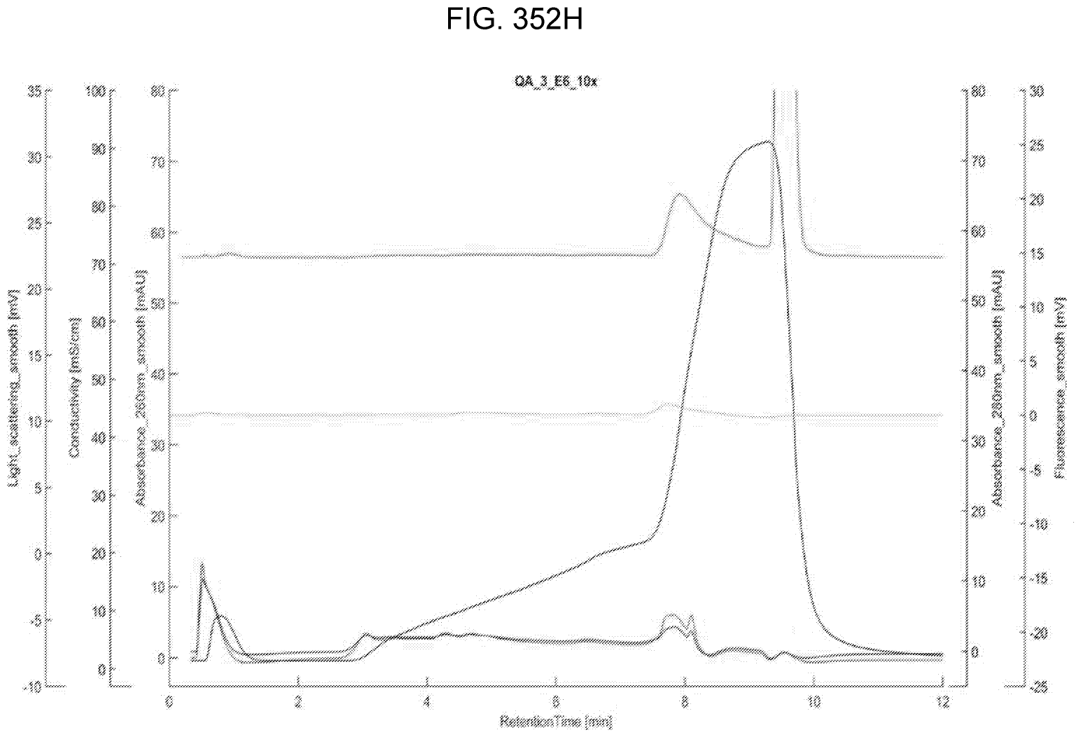

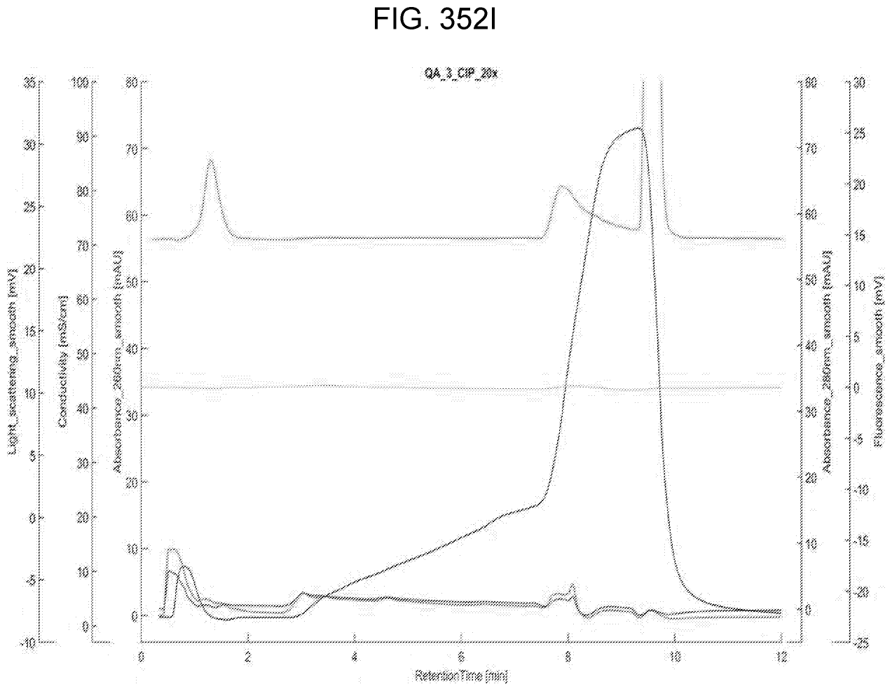

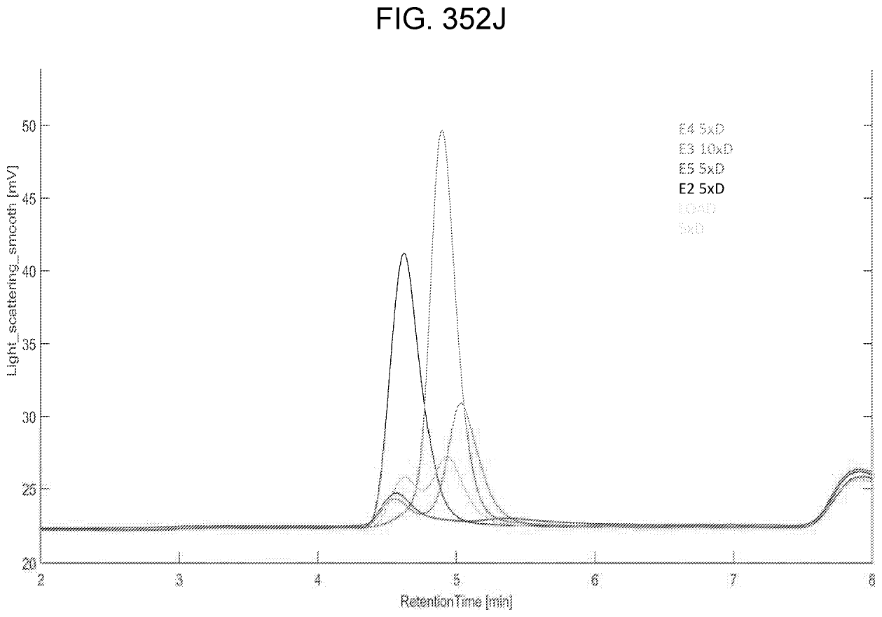

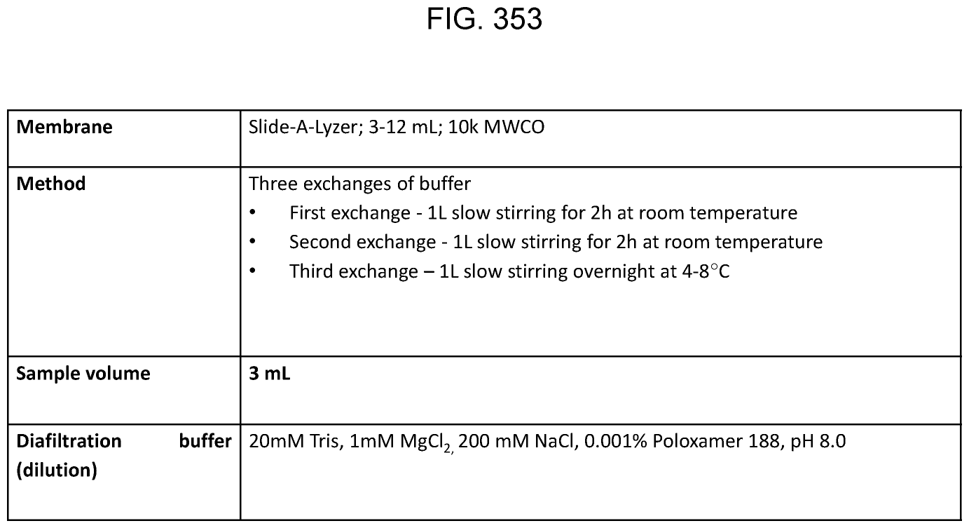

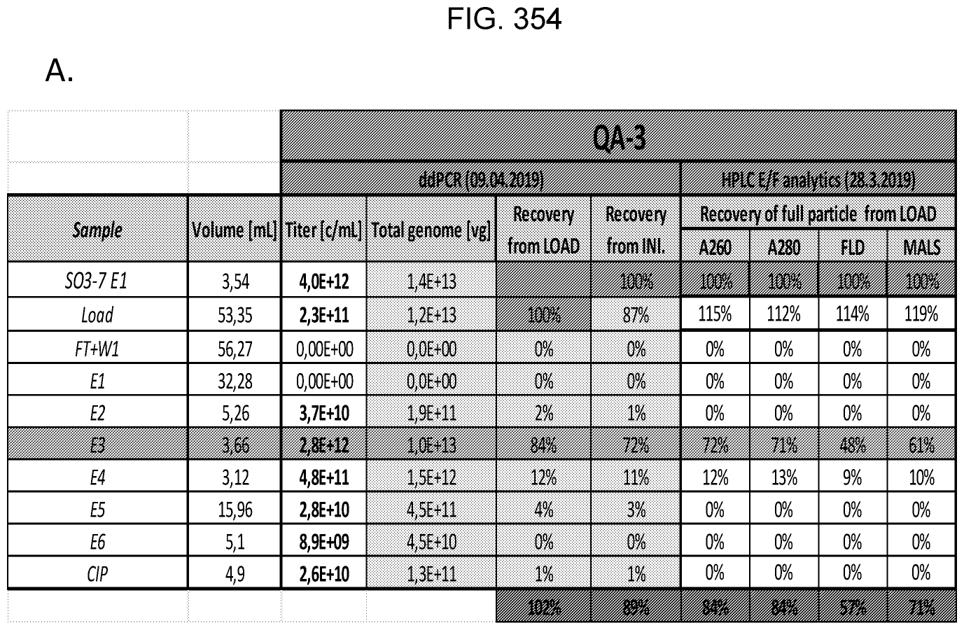

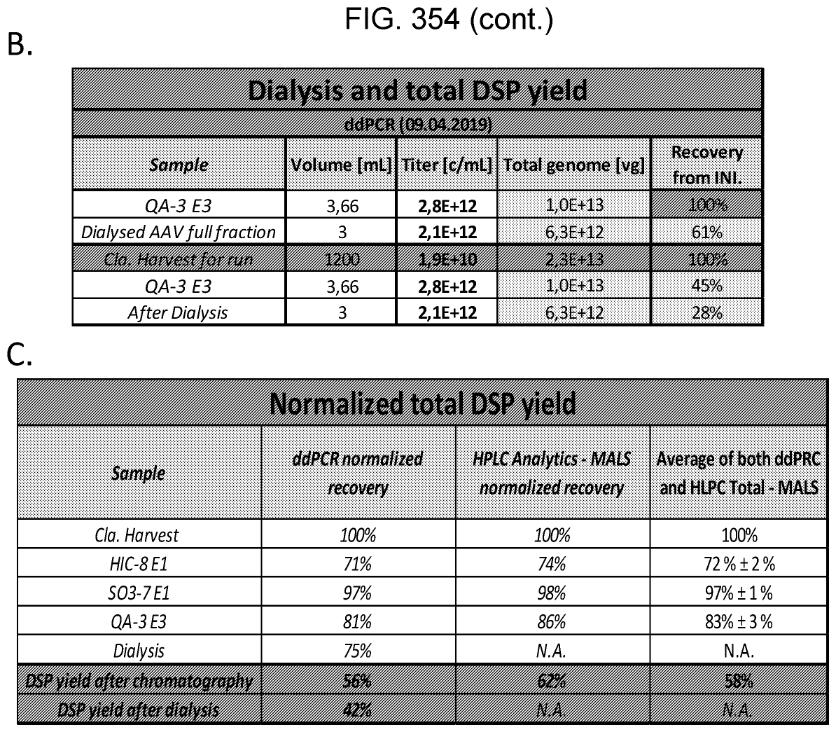

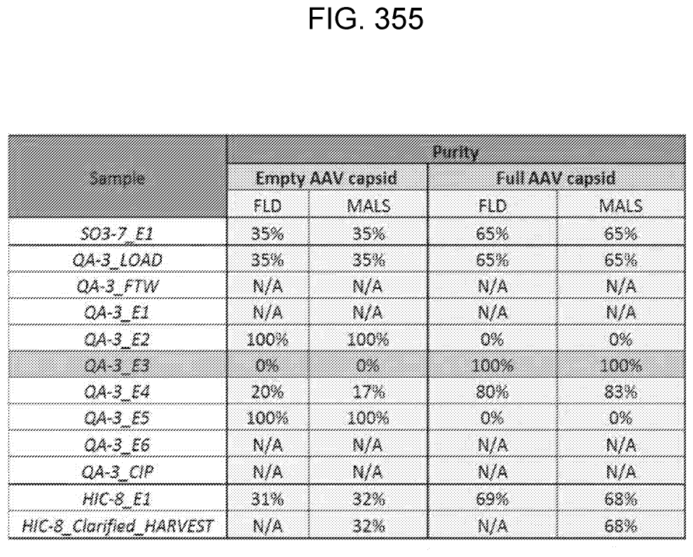

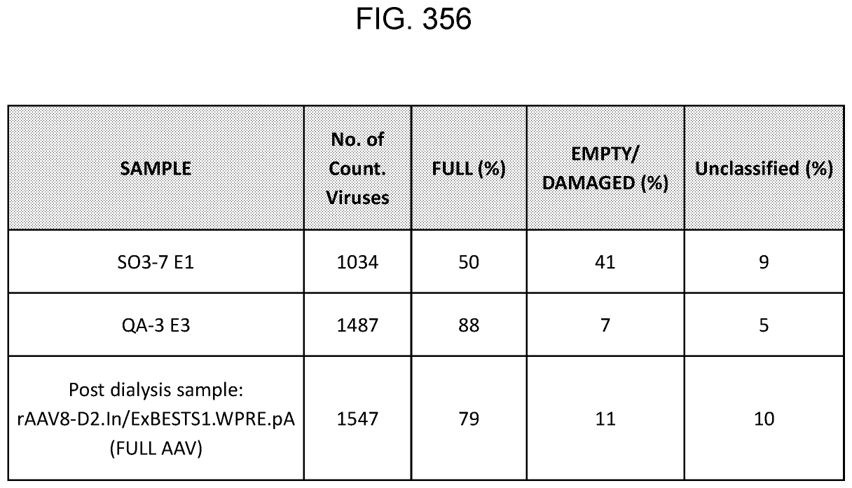

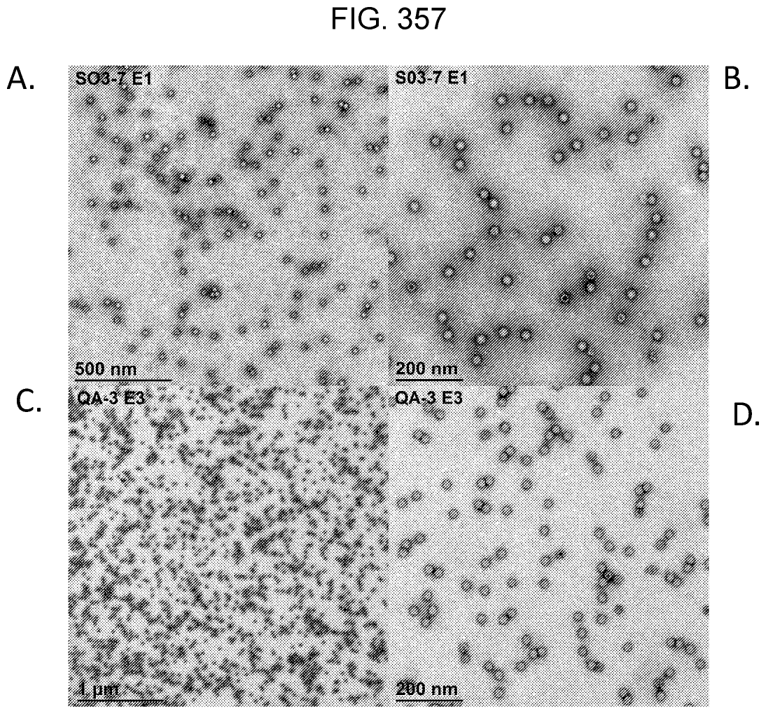

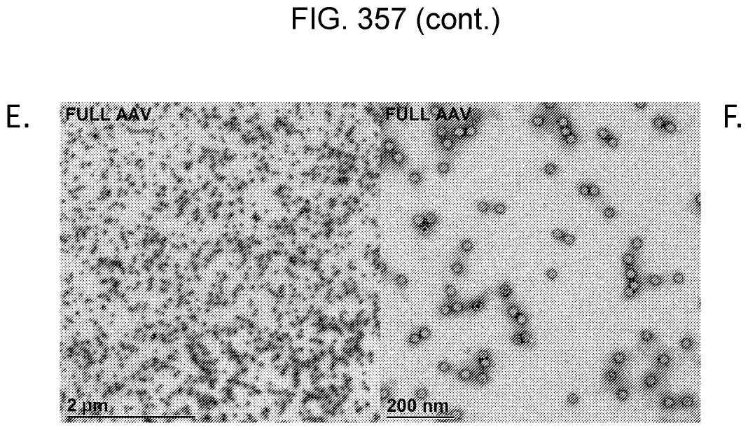

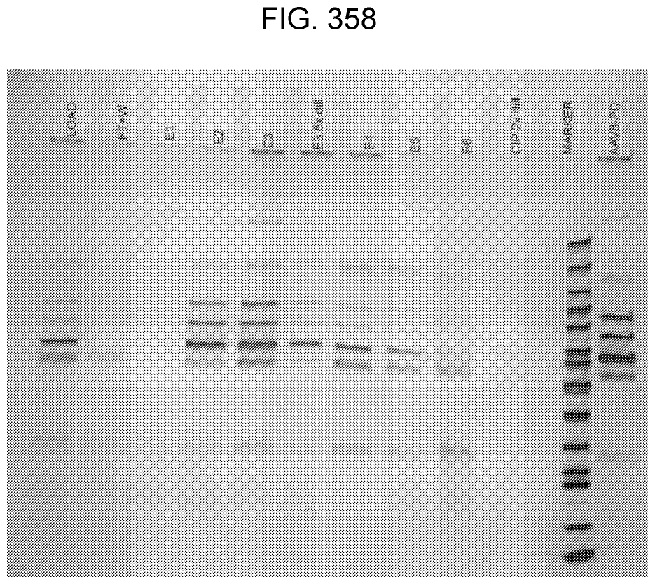

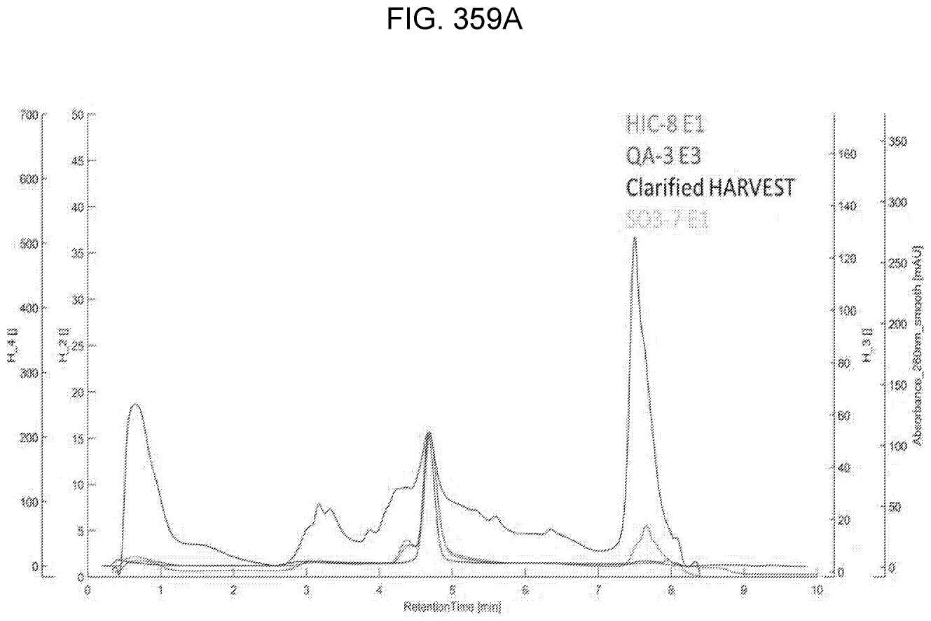

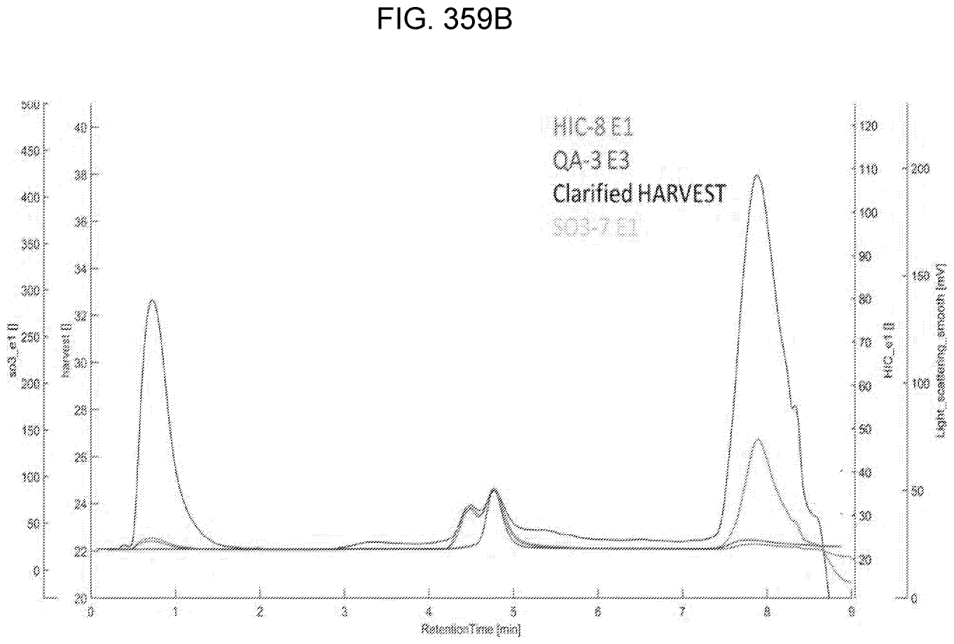

FIG. 178 is a schematic flow diagram of an exemplary passage procedure.

FIG. 179 is a table showing the generic guidance for the cell passage regime.

FIG. 180 is a table showing recommended reagent volumes (HBSS, cell dissociation solution and growth media) and cell seeding densities for cell passages.

FIG. 181 is a table showing materials/consumables used in the thaw and passage regimes.

FIG. 182 is a schematic flow diagram of the transient transfection and media harvest steps.

FIG. 183 is a table showing the volumes of chloroquine and media required for the initial media change, as a function of the production scale.

FIG. 184 is a table showing the parameters and operating ranges/setpoints for the transfection and harvest steps.

FIG. 185 is a table showing a guide to creating the calcium phosphate mediated transfection solution per 5×36-layer HYPERStacks®.

FIG. 186 is a table showing a schematic flow diagram of the filtration clarification step.

FIG. 187 is a table showing the parameters and operating ranges/setpoints for the clarification filtration step.

FIG. 188 is a table showing the materials/consumables used in the clarification filtration step.

FIG. 189 is a schematic flow diagram of a large scale tangential flow filtration unit operation.

FIG. 190 is a table showing the parameters and operating ranges/setpoints for the large scale tangential flow filtration step.

FIG. 191 is a table showing the materials/consumables used in the large scale tangential flow filtration step.

FIG. 192 is a schematic flow chart of iodixanol concentration unit operation.

FIG. 193 is a table showing the parameters and operating ranges/setpoints for the initial iodixanol concentration step.

FIG. 194 is a table showing the materials/consumables used in the centrifugation concentration step.

FIG. 195 is a schematic flow chart of the steps required to complete the iodixanol gradient purification step.

FIG. 196 is a table showing the parameters and operating ranges/setpoints for the iodixanol gradient purification step.

FIG. 197 is a table showing the key materials/consumables used in the iodixanol gradient purification step.

FIG. 198 is a schematic flow chart of cation exchange chromatography unit operation.

FIG. 199 is a table showing the parameters and operating ranges/setpoints for the cation exchange chromatography step.

FIG. 200 is a table showing the cation exchange chromatography operation conditions.

FIG. 201 is a table showing the materials/consumables used in the cation exchange chromatography step.

FIG. 202 is a schematic flow chart of the steps required to complete the small scale tangential flow filtration step.

FIG. 203 is a table showing the parameters and operating ranges/setpoints for the small scale tangential flow filtration step.

FIG. 204 is a table showing the key materials/consumables used in the small scale tangential flow filtration step.

FIG. 205 is a schematic flow chart of the sterile filtration and filling unit operations.

FIG. 206 is a table showing the parameters and operating ranges/setpoints for the sterile filtration and filling steps.

FIG. 207 is a table showing the materials/consumables used in the sterile filtration and filling steps.

FIG. 208 is a table showing the in-process hold points and storage conditions.

FIG. 209 is a table showing a list of preferred chemicals for solution preparation.

FIG. 210 is a table showing the sample formulated in clarified DMEM medium for Experiment A.

FIG. 211 is a table showing the buffers used for preparative and analytical runs for Experiment A.

FIG. 212 is a table showing SOP step gradients with dedicated buffers for HIC purification in Experiment A.

FIG. 213 is a table showing SOP step gradients with dedicated buffers for CEX purification in Experiment A.

FIG. 214 is a table showing SOP linear gradient from 0 to 100% mobile phase B in 60 column volumes (CVs) and then step to 100% MPC for 10 CVs for Experiment A.

FIG. 215 is a table showing the preparative run conditions for Experiment A.

FIG. 216 is a representative chromatogram from run HIC-25 for Experiment A. Entire run-loading phase (above), zoomed elution section (below). Legend: blue line is UV detection at 280 nm, red line is UV detection at 260 nm, brown line is conductivity, dark green line is pressure. Pressure rise during loading was 0.6 bar. Fractions are noted with brown markers. Main elution is E1. UV spike in loading phase corresponds to air bubble passing the column, which occurred after loading was stopped in order to transfer the sample to a smaller container.

FIG. 217 is a representative chromatogram based on HPLC analytics for Experiment A. Total method for HIC-25. A—blank (buffer) run; B—harvest; C—load; D—flow through (FT); E—wash 1 (W1); F—wash 2 (W2), G—elution (E1); H—wash 3 (W3); I—CIP; J—overlay of fluorescence signal. Legend: Legend: Fluorescence (Ex 280 nm, EM 348 nm): green curve, Absorbance at 260 nm: red curve, Absorbance at 280 nm: blue curve, Conductivity (mS/cm): black curve. Main elution (E1) is 10-fold diluted compared to other fractions. All chromatograms are on the same scale.

FIG. 218 is a table for recoveries of HIC-25 run based on ddPCR and HPLC total analytics for Experiment A.

FIG. 219 is a representative SDS-PAGE result for HIC-25 run for Experiment A. M—ladder. Fractions E1, W3 and CIP are 5-fold, 5-fold and 2-fold diluted, respectively. Main fraction is E1. VP1-VP3 proteins are marked by red rectangle.

FIG. 220 is a table showing preparative run conditions for E1 HIC-OH prepared to match binding conditions and loaded to CEX-SO3 column for Experiment A.

FIG. 221 is a representative chromatogram from run SO3-16 from Experiment A. Entire run-loading phase (above), zoomed elution section (below). Legend: blue line is UV detection at 280 nm, red line is UV detection at 260 nm, brown line is conductivity, dark green line is pressure. No pressure rise during loading. Fractions are noted with brown markers. Main elution is E1.

FIG. 222 is a representative chromatogram based on HPLC analytics from Experiment A. Total method for SO3-16. A—blank (buffer) run; B—Load BF; C—load; D—flow through+wash 1 (W1) (FT); E—wash 2 (W1); F—elution (E1); G—wash 3 (W3); H—CIP. Legend: Legend: Fluorescence (Ex 280 nm, EM 348 nm): green curve, Absorbance at 260 nm: red curve, Absorbance at 280 nm: blue curve, Conductivity (mS/cm): black curve. Main elution (E1) is 100-fold diluted where other fractions are 2.5-fold diluted or 5-fold diluted (W3 and CIP). All chromatograms are on the same scale.

FIG. 223 is a table showing recoveries based on ddPCR and HPLC Total analytics for preparative run SO3-16 for Experiment A.

FIG. 224 is a representative SDS-PAGE for SO3-16 run from Experiment A. M—ladder. Fraction E1, is 5-fold, and 10-fold diluted, fractions W3 and CIP are 2-fold diluted. Main fraction is E1. VP1-VP3 proteins are marked by red rectangle.

FIG. 225 is a table showing preparative run conditions for loading the entire elution (E1) from SO3-16 to AEX-QA (QA-14) column in Experiment A.

FIG. 226 is a representative chromatogram from run QA-14 for Experiment A. Entire run—loading phase (above), zoomed elution section (below). Legend: blue line is UV detection at 280 nm, red line is UV detection at 260 nm, brown line is conductivity, dark green line is pressure. No pressure rise during loading. Fractions are noted with brown markers. Main elution (full capsid AAV) is E3.

FIG. 227 is a representative chromatogram based on HPLC analytics Empty-full method for QA-14 for Experiment A. A—SO3-16 E1; B—FT+W; C—E1; D—E2 (empty AAV capsids); E—E3 (full AAV capsids); F—E4 (tail portion of main full peak); G—E5; H—E6, I—CIP. Legend: Legend: Fluorescence (Ex 280 nm, EM 348 nm): green curve, Absorbance at 260 nm: red curve, Absorbance at 280 nm: blue curve, Conductivity (mS/cm): black curve. Pictures A, B, C, F, G, H and I are on the same scale, D is on 2-fold larger scale and E in on 4 fold larger scale. Fractions are 20-fold diluted (picture A) or 10-fold (picture H) others are 5-fold diluted. Ratios A260/A280 are presented on the corresponding fractions.

FIG. 228 is a table showing concentration and buffer exchange conditions by implementation of TFF on QA-14 E3 sample for Experiment A.

FIG. 229 shows a table of recoveries based on ddPCR and HPLC E/F analytics for preparative run QA-14 TFF and total DSP yield from Experiment A.

FIG. 230 shows a table of purity of both empty and full AAV capsids based on HPLC E/F analytics for Experiment A.

FIG. 231 shows a table of the ratio of full and empty AAVs evaluated by TEM for Experiment A.

FIG. 232 shows representative fractions from QA-14 after TFF evaluated by TEM for Experiment A. E3 fraction (above), E2 fraction (below).

FIG. 233 shows a representative SDS-PAGE result for QA-14 run for Experiment A. M—ladder. Fraction E3 is neat and 5-fold diluted, others are neat. Main fraction is E3. AAV8 FULLS is E3 fraction after TFF. VP1-VP3 proteins are marked by red rectangle.

FIG. 234 is a table showing the sample formulated in clarified DMEM medium for Experiment B.

FIG. 235 is a table showing the buffers used for preparative and analytical runs for Experiment B.

FIG. 236 is a table showing SOP step gradients with dedicated buffers for HIC purification in Experiment B.

FIG. 237 is a table showing SOP step gradients with dedicated buffers for CEX purification in Experiment B.

FIG. 238 is a table showing SOP linear gradient from 0 to 100% mobile phase B in 60 column volumes (CVs) and then step to 100% MPC for 10 CVs for Experiment B.

FIG. 239 is a table showing the preparative run conditions for Experiment B.

FIG. 240 is a representative chromatogram from run HIC-26 for Experiment B. Entire run—loading phase (above), zoomed elution section (below). Legend: blue line is UV detection at 280 nm, red line is UV detection at 260 nm, brown line is conductivity, dark green line is pressure. Pressure rise during loading was 0.5 bar. Fractions are noted with brown markers. Main elution is E1.

FIG. 241 is a representative chromatogram based on HPLC analytics for Experiment B. Total method for HIC-26. A—blank (buffer) run; B—harvest; C—load; D—flow through (FT); E—wash 1 (W1); F—wash 2 (W2), G—elution (E1); H—wash 3 (W3); I—CIP; J—overlay of fluorescence signal. Legend: Legend: Fluorescence (Ex 280 nm, EM 348 nm): green curve, Absorbance at 260 nm: red curve, Absorbance at 280 nm: blue curve, Conductivity (mS/cm): black curve. Main elution (E1) is 10-fold diluted compared to other fractions. All chromatograms are on the same scale.

FIG. 242 is a table for recoveries of HIC-26 run based on ddPCR and HPLC total analytics for Experiment B.

FIG. 243 is a representative SDS-PAGE result for HIC-26 run for Experiment B. M—ladder. Fractions E1, W3 and CIP are 5-fold, 5-fold and 2-fold diluted, respectively. Main fraction is E1. VP1-VP3 proteins are marked by red rectangle.

FIG. 244 is a table showing preparative run conditions for E1 HIC-OH prepared to match binding conditions and loaded to CEX-SO3 column for Experiment B.

FIG. 245 is a representative chromatogram from run SO3-17 from Experiment B. Entire run—loading phase (above), zoomed elution section (below). Legend: blue line is UV detection at 280 nm, red line is UV detection at 260 nm, brown line is conductivity, dark green line is pressure. No pressure rise during loading. Fractions are noted with brown markers. Main elution is E1.

FIG. 246 is a representative chromatogram based on HPLC analytics from Experiment B. Total method for SO3-17. A—blank (buffer) run; B—Load BF; C—load; D—flow through+wash 1 (W1) (FT); E—wash 2 (W1); F—elution (E1); G—wash 3 (W3); H—CIP. Legend: Legend: Fluorescence (Ex 280 nm, EM 348 nm): green curve, Absorbance at 260 nm: red curve, Absorbance at 280 nm: blue curve, Conductivity (mS/cm): black curve. Main elution (E1) is 100-fold diluted where other fractions are 2.5-fold diluted or 5-fold diluted (W3 and CIP). All chromatograms are on the same scale.

FIG. 247 is a table showing recoveries based on ddPCR and HPLC Total analytics for preparative run SO3-17 for Experiment B.

FIG. 248 is a representative SDS-PAGE for SO3-17 run from Experiment B. M—ladder. Fraction E1, is 5-fold, and 10-fold diluted, fractions W3 and CIP are 2-fold diluted. Main fraction is E1. VP1-VP3 proteins are marked by red rectangle.

FIG. 249 is a table showing preparative run conditions for loading the entire elution (E1) from SO3-17 to AEX-QA (QA-15) column in Experiment B.

FIG. 250 is a representative chromatogram from run QA-15 for Experiment B. Entire run—loading phase (above), zoomed elution section (below). Legend: blue line is UV detection at 280 nm, red line is UV detection at 260 nm, brown line is conductivity, dark green line is pressure. No pressure rise during loading. Fractions are noted with brown markers. Main elution (full capsid AAV) is E3.

FIG. 251 is a representative chromatogram based on HPLC analytics Empty-full method for QA-15 for Experiment B. A—SO3-16 E1; B—FT+W; C—E1; D—E2 (empty AAV capsids); E—E3 (full AAV capsids); F—E4 (tail portion of main full peak); G—E5; H—E6, I—CIP. Legend: Legend: Fluorescence (Ex 280 nm, EM 348 nm): green curve, Absorbance at 260 nm: red curve, Absorbance at 280 nm: blue curve, Conductivity (mS/cm): black curve, multi angle light scattering detector (MALS) is pink curve. Pictures B, C, G, H and I are on the same scale, A, D, E and F are on 2-fold larger scale. Fractions are 20-fold diluted (picture A) or 10-fold (picture H) others are 5-fold diluted. Ratios A260/A280 are presented on the corresponding fractions.

FIG. 252 is a table showing concentration and buffer exchange conditions by implementation of TFF on QA-15 E3 sample for Experiment B.

FIG. 253 shows a table of recoveries based on ddPCR and HPLC E/F analytics for preparative run QA-15 TFF and total DSP yield from Experiment B.

FIG. 254 shows a table of purity of both empty and full AAV capsids based on HPLC E/F analytics for Experiment B.

FIG. 255 shows a table of the ratio of full and empty AAVs evaluated by TEM for Experiment B.

FIG. 256 shows representative fractions from QA-15 after TFF evaluated by TEM for Experiment B. QA-15 E3 fraction (above); E5 fraction (below).

FIG. 257 shows a representative SDS-PAGE result for QA-15 run for Experiment B. M—ladder. Fraction E3 is neat and 5-fold diluted, others are neat. Main fraction is E3. AAV8 FULLS is E3 fraction after TFF. Genscript Express Plus 4-20% gel was used.

FIG. 258 shows a representative HPLC chromatogram Fingerprint Method from Experiment B. Overlay of each chromatographic stage is presented. A: overlay of harvest and main eluate of HIC-OH step. HIC eluate is 60-fold diluted compared to harvest. B: Overlay of harvest and main SO3 eluate (E1). SO3 eluate is 200-fold diluted compared to harvest material. C: overlay of harvest, QA load and QA main eluate (E3). Load is 10-fold and E3 is 60-fold diluted compared to harvest. All chromatograms are on the same scale. Y-axis is absorbance at 260 nm.

FIG. 259 is a table showing HIC (OH) chromatography conditions for ABCA4.

FIG. 260A-B is a representative HIC (OH) chromatogram and vector recovery analysis for ABCA4. (A) Zoomed elution section of chromatogram is shown. Elution fragment is indicated with brackets. (B) Vector recoveries in the HIC fractions as measured by HPLC total particle analytics. HIC elustion step optimization required to increase overall step yield.

FIG. 261 is a table showing CEX (SO3) chromatography conditions for ABCA4. All fractions neutralized with addition of 1M Tris, pH9.0; 10% of total fraction volume was added.

FIG. 262A-B is a representative CEX (SO3) chromatogram and vector analysis recovery for ABCA4. (A) shows zoomed elution. (B) shows vectors recovered in the SO3 fractions as measured by HPLC total particle analysis.

FIG. 263 is a table showing AEX (QA) chromatography conditions for ABCA4. All fractions neutralized with addition of 1M BTP, pH 6.5; 5% of total fraction volume added.

FIG. 264A-B is a representative AEX (QA) chromatogram and vector recovery analysis for ABCA4. (A) shows zoomed elution with empty and full particles shows in brackets. (B) shows vector recoveries of empty particles (top) and full particles (bottom) in the QA fractions as measured by total particle HPLC analytics.

FIG. 265 is a table showing purity of (Full:Empty) particles based on HPLC analytics for ABCA4. Optimal representation of purity (E/F) ratio is given by FLD and MALS detectors. Enrichment from approximately 55%-94% of full AAV particles is achieved by QA step.

FIG. 266A-B is a representative purity of particles (Full:Empty) based on TEM for ABCA4. (A) shows a table of sample details (B) shows sample purified with iodixanol (AAV8Y733F) (two left panels) and sample purified by QA chromatography (AAV8 QA-1 E3) (two right panels).

FIG. 267 is a representative particle purification by SDS-PAGE analysis for ABCA4.

FIG. 268 is a schematic flow diagram showing the HIC chromatography unit operation for ABCA4.

FIG. 269 is a table showing parameter and operating ranges for the HIC capture step for ABCA4.

FIG. 270 is a table showing HIC chromatography operating parameters for ABCA4.

FIG. 271 is a representative chromatogram of the HIC step for ABCA4; including the loading, washes, elution and CIP stages. Legend: Flow through (F1), Post-load wash (W1), post-load wash 2 (W2), elution (E1), post-elution wash (W3), cleaning in place (CIP).

FIG. 272 is a representative zoomed in chromatogram of the HIC step for ABCA4. Legend: Post-load wash (W1), post-load wash 2 (W2), elution (E1), post-elution wash (W3), cleaning in place (CIP).

FIG. 273 is a table showing HIC buffer composition and target specifications for ABCA4.

FIG. 274 is a table showing details of the key materials and consumables that are to be utilised in the HIC chromatography step for ABCA4.

FIG. 275 is a schematic flow diagram showing the SO3 chromatography unit operation for ABCA4.

FIG. 276 is a table showing parameter and operating ranges/setpoints for SO3 chromatography step for ABCA4.

FIG. 277 is a table showing individual chromatography steps and operating parameters for ABCA4.

FIG. 278 is a representative typical full SO3 chromatogram run for ABCA4.

FIG. 279 is a representative zoomed in elution section of the chromatogram for ABCA4. Red rectangle marks the main elution peak. Legend: post-load wash 2 (W2), elution (E1), post-elution wash (W3), cleaning in place (CIP).

FIG. 280 is a table showing SO3 buffer compositions used for ABCA4.

FIG. 281 is a table showing key materials/consumables used in the centrifugation concentration step for ABCA4.

FIG. 282 is a schematic flow diagram showing the QA chromatography unit operation process flow for ABCA4.

FIG. 283 is a table showing the parameters and associated operating ranges or setpoints which are to be used for the QA chromatography step for ABCA4.

FIG. 284 is a table showing specific steps associated with the chromatography run for ABCA4.

FIG. 285 is a representative full QA chromatogram of the linear gradient elution for ABCA4.

FIG. 286 is a representative QA Chromatogram zoomed onto the gradient elution. E2—empty particles. E3—full particles. E4—peak tail containing a mixture of full, empty and damaged particles.

FIG. 287 is a table showing QA buffer composition and target specifications for ABCA4.

FIG. 288 is a table showing key materials/consumables used in the QA chromatography unit operation for ABCA4.

FIG. 289 is a schematic diagram of a flow chart of the tangential flow filtration unit operation for purification an AAV-ABCA4 vector.

FIG. 290 is a table listing exemplary parameters and associated operating ranges or setpoints which may be used for the TFF run for purification an AAV-ABCA4 vector.

FIG. 291 is a table providing exemplary materials and consumables that may be used in the tangential flow filtration unit operation for purification an AAV-ABCA4 vector.

FIG. 292 is a table providing exemplary hold times at in-process points that may used during the manufacture of the AAV-ABCA4 product.

FIG. 293 is a schematic diagram showing upstream and downstream transgene structures that combine to form a complete ABCA4 transgene.

FIG. 294 is a schematic diagram showing overlap C sequence with out-of-frame AUG codons prior to an in-frame AUG codon.

FIG. 295 is a schematic showing predicted secondary structures of overlap zones C and B.

FIG. 296 is a schematic diagram showing example overlapping vectors.

FIG. 297A-D is a series of diagrams of transgene outcomes following transduction with an ABCA4 overlapping dual vector system. (A) Upstream and downstream transgene single-stranded DNA forms. These can anneal by single-strand annealing (SSA) via their regions of homology on complementary transgenes (B), following which the complete recombined large transgene can be generated (C). Abbreviations: CDS=coding sequence; DSB=double-stranded break; HR=homologous recombination; ITR=inverted terminal repeat; pA=polyA signal; SSA=single-strand annealing; WPRE=Woodchuck hepatitis virus post-transcriptional regulatory element.

FIG. 298 is a schematic diagram showing overlapping upstream and downstream dual vectors.

FIG. 299 is a series of diagrams showing the overlapping upstream and downstream dual vectors.

FIG. 300 is a diagram showing dual vector upstream and downstream variants A, B, C, D, E, F, G and X, that may be comprised in either AAV2/8 Y733F ABCA4 or AAV2/8-ABCA4 are shown. Full length or truncated versions of ABCA4 (tABCA4) were influenced by the overlapping region of the dual vector system.

FIG. 301 is a schematic diagram showing dual vector overlap variants. Nucleotides of the ABCA4 coding sequence (SEQ ID NO: 11) are included in each transgene are shown.

FIG. 302 is a diagram showing a segment of nucleotide sequence from the upstream transgene variant B. The sequence from the SwaI site was consistent in all upstream transgene variants and the features of a possible cryptic poly A signal are highlighted.

FIG. 303 is a pair of diagrams of the development of the ABCA4 dual vector system. A. Different aspects of vector design were considered and assessed, including the genetic elements and structure of the transgene and the vector capsid and dose. B. Dual vector variants carrying different overlap lengths were compared to determine the optimal region for recombination between two transgenes. AAV=adeno-associated virus; ABCA4=ATP-binding cassette transporter protein family member 4; Do=downstream transgene variant; GRK1=human rhodopsin kinase promoter; In=intron; ITR=inverted terminal repeat; pA=polyA signal; Up=upstream transgene variant; WPRE=Woodchuck hepatitis virus post-transcriptional regulatory element.

FIG. 304A-B are schematic diagrams showing (A) A forward primer binding ABCA4 CDS provided by the upstream transgene and a reverse primer binding ABCA4 CDS in the downstream transgenes were used to amplify transcripts from recombined transgenes. Amplicons were sequenced to confirm the correct ABCA4 CDS was contained across the overlap regions of the transcripts. (B) A forward primer binding downstream of the predicted GRK1 transcriptional start site (TSS) and a reverse primer binding within the upstream ABCA4 CDS were used to assess transcript forms from dual vector C injected eyes and dual vector 5′C injected eyes.

FIG. 305 is a diagram of promoters and additional sequences that can be used to drive expression of the ABCA4 upstream sequence. RK=GRK1 promoter, IntEx=intron and exon sequence, CMV=cytomegalovirus early enhancer; CBA=chicken beta actin promoter; SA/SD=splice acceptor and splice donor.

FIG. 306 is a diagram of AAV vectors used to express the ABCA4 upstream sequence or GFP. ITR=Inverted Terminal Repeat, WPRE=Woodchuck hepatitis virus post-transcriptional regulatory element, GFP=green fluorescent protein, IntEx=intron and exon sequence, CBA=chicken beta actin promoter, CMV=cytomegalovirus enhancer, RK=rhodopsin kinase promoter (GRK1 promoter), RBG=Rabbit beta globin, SA/SD=splice acceptor and splice donor sequence.

FIG. 307 is a sequence of a CMVCBA.In.GFP.pA vector (SEQ ID NO: 17).

FIG. 308 is a sequence of a CMVCBA.GFP.pA vector (SEQ ID NO: 18).

FIG. 309 is a sequence of a CBA.IntEx.GFP.pA vector (SEQ ID NO: 19).

FIG. 310 is a sequence of a CAG.GFP.pA vector (SEQ ID NO: 20).

FIG. 311 is a sequence of an AAV.5′CMVCBA.In.ABCA4.WPRE.kan vector (SEQ ID NO: 21).

FIG. 312 is a sequence of an AAV.5′CMVCBA.ABCA4.WPRE.kan vector (SEQ ID NO: 22).

FIG. 313 is a sequence of an AAV.5′CBA.IntEx.ABCA4.WPRE.kan vector (SEQ ID NO: 23).

FIG. 314 is a series of schematic diagrams depicting exemplary ABCA4 expression constructs of the disclosure.

FIG. 315 is a sequence of the ITR to ITR portion of pAAV.RK.5′ABCA4.kan (SEQ ID NO: 26), comprising a sequence encoding a 5′ ITR (SEQ ID NO: 27), a sequence encoding an RK promoter (SEQ ID NO: 28), a sequence encoding a Rabbit Beta-Globin (RBG) Intron/Exon (Int/Ex) (SEQ ID NO: 39), a sequence encoding a 5′ portion of the coding sequence of an ABCA4 gene (SEQ ID NO: 29), and a sequence encoding a 3′ ITR (SEQ ID NO: 30).

FIG. 316 is a sequence of the ITR to ITR portion of pAAV.3′ABCA4.WPRE.kan (SEQ ID NO: 30), comprising a sequence encoding a 5′ ITR (SEQ ID NO: 27), a sequence encoding a 3′ portion of the coding sequence of an ABCA4 gene (SEQ ID NO: 31), a sequence encoding WPRE (SEQ ID NO: 32), a sequence encoding bGH polyA and a sequence encoding a 3′ ITR (SEQ ID NO: 33).

FIG. 317A-C are a series of pictures showing the conversion of a transgene encoded by a double stranded DNA (dsDNA) to single stranded sense and antisense DNAs (ssDNA), and encapsidation of the ssDNAs in AAV viral particles.

FIG. 318A-D are a series of pictures showing the uptake of the AAV viral particles containing the sense and antisense ssDNAs by the nucleus (A), release of the sense and antisense strands from the viral particles (B), synthesis of the complementary strand to regenerate dsDNA (C) and transcription of the transgene (D).

FIG. 319A-H are a series of pictures that depict encapsidation, transduction, and reformation of a large transgene in an AAV dual vector system through single strand annealing and second strand synthesis. The large transgene is initially encoded as dsDNA (A-B). Subsequently, ssDNAs of overlapping 5′ and 3′ fragments of the large transgene are encapsidated by AAV viral particles (C). Viral particles comprising complementary strands of the 5′ and 3′ fragments of the large transgene are generated, and these ssDNAs comprise a region of complementary, overlapping sequence (shown in red). In this example, the antisense ssDNA of the 5′ fragment and the sense strand of the 3′ are depicted. AAV particles comprising the ssDNAs are transduced (D), and the ssDNAs are released from the viral particles into the nucleus (E). The 5′ and 3′ fragments hybridize at the complementary, overlapping sequence in the nuclear environment (F), a dsDNA of the entire large transgene is generated through second strand synthesis (G), and this dsDNA is subsequently transcribed and the transgene expressed (H).

FIG. 320 is an outline of an ABCA4 overlapping dual vector system of the disclosure. The elements of an adeno-associated virus (AAV) transgene were split across two independent transgenes, “upstream” and “downstream”. The upstream transgene contained the promoter and upstream fragment of ABCA4 coding sequence whilst the downstream transgene carried the downstream fragment of ABCA4 coding sequence plus a WPRE and a bovine growth hormone (bGH) pA signal. In the optimized overlapping dual vector system depicted, both transgenes carried a 207 bp region of overlap formed from ABCA4 coding sequence bases 3,494-3,701. Once inside the same host cell nucleus, the two transgenes align and recombine via the region of overlap. ABCA4=ATP-binding cassette transporter protein family member 4; GRK1=human rhodopsin kinase promoter; In=intron; ITR=inverted terminal repeat; pA=polyA signal; WPRE=Woodchuck hepatitis virus post-transcriptional regulatory element.

FIG. 321 is a table showing transgene details for the dual vector combinations tested. The final row contains the details for the optimized overlapping dual vector system. ABCA4=ATP-binding cassette transporter protein family member 4; bp=base pairs; CDS=coding DNA sequence; GRK1=human rhodopsin kinase promoter; pA=polyA signal; WPRE=Woodchuck hepatitis virus post-transcriptional regulatory element.

FIG. 322 is a schematic diagram depicting an overview of the downstream and fill and finish steps of the manufacturing process for AAV-ABCA4, upstream and/or downstream vectors.

FIG. 323A-B is a representative optimized HIC chromatogram. Both optimized peak cutting annotation (1.02M buffer) and non-optimized peak cutting annotation (1.08M buffer) is shown. Key: W2=post load wash 2, E1=elution fraction, W3=post elution buffer.

FIG. 324A-B is a representative optimized CEX chromatogram. Both optimized peak cutting annotation (1.33M buffer) and non-optimized peak cutting annotation (1.3M buffer) is shown. Key: W2=post load wash 2, E1=elution fraction, W3=post elution buffer.

FIG. 325A-C is a series of representative optimized condition run through chromatograms for the HIC, CEX, and QA steps, respectively.

FIG. 326 is a table detailing step recoveries for the optimization process.

FIG. 327A is a table detailing Full:Empty AAV results over the QA separation by MALS. FIG. 327B is a table detailing Full:Empty AAV results over the QA separation by MALS and TEM.

FIG. 328A-D is a proof of concept table and a series of three graphs providing data from four confirmatory transfection and purification runs for AAV-RPGR dual vectors, however, the transfection and purification runs can be used with any transgene, including ABCA4. Four transfection conditions (A) were evaluated, following on from results of an initial scoping study. The number of vector particles (Capsid ELISA) and the number of particles that contain the genome insert (Genomic titre) were quantified for each condition (B).

FIG. 329A-B is a proof of concept graph (A) and a table (B) depicting a quantification of an orthogonal method of evaluating full:empty ratios for AAV-RPGR, however, the orthogonal method of evaluating full:empty ratios can be used with any transgene, including ABCA4. The full particle analysis, presented at FIG. 328, may underestimate the actual values, however the trends are valid. Therefore, samples from the four conditions were further measured by an orthogonal method. The results from the orthogonal method mirrored the trend seen from the full particle analysis (FIG. 328). A comparison with an earlier result, from material generated with a different transfection agent (CaPO₄), suggests that the choice of transfection agent may also have an effect on the ratio of full to empty particles.

FIG. 330 is a graph depicting the effect of transfection reagent (PEI vs. CaPO₄) on AAV full:empty vector ratios. A PEI vs. CaPO₄ comparison transfection study generated material that was analyzed for full:empty vector ratios using HPLC. As with previous analysis, the material had not been through a process step that would enrich for full particles. Previous variable conditions that were kept constant between the two transfection conditions were total DNA, PEI/DNA ratio and ratio of transfection plasmids. For each of the two transfection reagents, the left bar is FLD, and the right bar is MALS.

FIG. 331 is an annotated sequence of an illustrative plasmid pAAV.stbIR.3′ABCA4.WPRE.kan (SEQ ID NO: 41), comprising a sequence encoding a 5′ ITR (AAV2 derived ITR, nucleotides 16-130, SEQ ID NO: 42), a sequence encoding a 3′ABCA4 (nucleotides 176-3509, SEQ ID NO: 43), a sequence encoding a WPRE (nucleotides 3516-4108, SEQ ID NO: 44), a sequence encoding a BGH PolyA (nucleotides 4115-4278, SEQ ID NO: 45), and a sequence encoding a 3′ IR (AAV derived ITR, nucleotides 4422-4542, SEQ ID NO: 46). In certain embodiments, the ITR comprises or consists of nucleotides 1-130, the 3′ABCA4-encoding sequence comprises or consists of nucleotides 181-3509, the WPRE comprises or consists of nucleotides 3522-4110, and/or the BGH PolyA comprises or consist of nucleotides 4115-4383. IR=ITR.

FIG. 332 is an annotated sequence of an illustrative plasmid pAAV.stbITR.CBA.InEx.5′ABCA4.kan (SEQ ID NO: 47), comprising a sequence encoding a 5′ IR (AAV2 derived ITR, nucleotides 16-130, SEQ ID NO: 48), a sequence encoding a CBA promoter (nucleotides 190-467, SEQ ID NO: 49), a sequence encoding an intron (nucleotides 468-590, SEQ ID NO: 50), a sequence encoding an exon (nucleotides 591-630, SEQ ID NO: 51), 5′ABCA4 (nucleotides 650-4351, SEQ ID NO: 52), and a sequence encoding a 3′ IR (AAV2 derived ITR, nucleotides 4389-4509, SEQ ID NO: 53). In certain embodiments, the ITR comprises or consists of nucleotides 1-130, the CBA promoter comprises or consists of nucleotides 186-468, the InEx comprises or consists of nucleotides 469-643, and the 5′ABCA4 comprises or consists of nucleotides 650-4350. IR=ITR.

FIG. 333 is an annotated sequence of an illustrative plasmid pAAV.stbITR.CBA.RBG.5′ABCA4.kan (SEQ ID NO: 54), comprising a sequence encoding a 5′ IR (AAV2 derived ITR, nucleotides 16-130, SEQ ID NO: 55), a sequence encoding a CBA promoter (nucleotides 190-467, SEQ ID NO: 56), a sequence encoding a RGB intron (nucleotides 704-876, SEQ ID NO: 57), a sequence encoding a 5′ABCA4 (nucleotides 919-4620, SEQ ID NO: 58), and a sequence encoding a 3′ IR (nucleotides 4667-4788, SEQ ID NO: 59). In certain embodiments, the ITR comprises or consists of nucleotides 1-130, the CBA comprises or consists of nucleotides 186-468, the RGB comprises or consists of nucleotides 469-881, the 5′ABCA4 comprises or consists of nucleotides 919-4619, and the 3′ITR comprises or consists of nucleotides 4658-4778. IR=ITR.

FIG. 334 is an annotated sequence of an illustrative plasmid pAAV.stbITR.CMV.CBA.5′ABCA4.kan (SEQ ID NO: 60), comprising a sequence encoding a 5′ IR (AAV2 derived ITR, nucleotides 16-130, SEQ ID NO: 61), a sequence encoding a CMV enhancer (nucleotides 322-556, SEQ ID NO: 62), a sequence encoding a CBA promotor (nucleotides 571-849, SEQ ID NO: 63), a sequence encoding a 5′ABCA4 (nucleotides 856-4557, SEQ ID NO: 64), and a sequence encoding a 3′ IR (nucleotides 4667-4788, SEQ ID NO: 65). In some embodiments, the ITR comprises or consists of nucleotides 1-130, the CMV sequence comprises or consists of nucleotides 186-568, the CBA sequence comprises or consists of nucleotides 569-849, the 5′ABCA4 comprises or consists of nucleotides 556-4556, and the 3′ITR comprises or consists of nucleotides 4595-4715. IR=ITR.

FIG. 335 is an annotated sequence of an illustrative plasmid pAAV.stbITR.RK.5′ABCA4.kan (SEQ ID NO: 66), comprising a sequence encoding a 5′ IR (AAV2 derived ITR, nucleotides 16-130, SEQ ID NO: 67), a sequence encoding a RK promoter (nucleotides 186-384, SEQ ID NO: 68), a sequence encoding a 5′ABCA4 (nucleotides 576-4267, SEQ ID NO: 69), and a sequence encoding a 3′ IR (nucleotides 4275-4425, SEQ ID NO: 70).

FIG. 336 provides a description of buffers for ABCA4 HIC (FIG. 336A), CEX (FIG. 336B), and AEX (FIG. 336C) preparative runs, and analytical runs (FIG. 336D).

FIG. 337 is a table showing HIC chromatography conditions for ABCA4 preparative runs.

FIG. 338 is a table showing CEX (SO3) chromatography conditions for ABCA4 preparative runs.

FIG. 339 is a table showing AEX chromatography conditions for ABCA4 preparative runs.

FIG. 340 is a table showing conditions for a capture step on HIC using OH columns HPLC analytical methods. For the preparative runs, clarified harvest material (1.2 L—divided in two bottles each containing 0.6 L) was thawed at room temperature, pooled and diluted 1:1 (1.2 L harvest+1.2 L buffer) with dilution buffer. Loading to the column using system pump at 5 CV/min. Tech transfer run was the eight (8) run for HIC conditions (HIC-8).

FIGS. 341A and 341B are chromatograms from run HIC-8 with entire run-loading phase (FIG. 341A) and zoomed elution section (FIG. 341B). For FIG. 341A at 1000, the top line is UV detection at 260 nm, the next line down is conductivity, the next line down is UV detection at 280 nm, and the lowest line is pressure. For FIG. 341B at about 2400, the highest peak is UV detection at 280 nm, the second highest peak is UV detection at 260 nm, and the lower line is conductivity. Pressure rise during loading was 0.3 bar. Fractions are noted with markers. Main elution is E1.

FIGS. 342A-J show chromatograms based on HPLC analysis—total method for HIC-8. FIG. 342A—blank (buffer) run; FIG. 342B—harvest; FIG. 342C—load; FIG. 342D—flow through (FT); FIG. 342E—wash 1 (W1); FIG. 342F—wash 2 (W2); FIG. 342G—elution (E1); FIG. 342H—wash 3 (W3); FIG. 342I—CIP; FIG. 342J—overlay of fluorescence and MALS signal. For each graph of FIGS. 342A-I, the x-axis shows retention time (minutes), and the y-axis shows absorbance, conductivity and light scattering. The line originating around the middle of the y-axis is fluorescence (Ex 280 nm, EM 348 nm); the two lines originating around the bottom of the y-axis are absorbance at 260 nm and absorbance at 280 nm; and the line peaking about 10 minutes retention time is conductivity (mS/cm). Main elution (E1) is 10-fold diluted compared to the other fractions. All chromatograms are on the same scale.

FIG. 343 is a table showing recoveries of HIC-8 run based on ddPCR and HPLC total analytics.

FIG. 344 shows SDS-PAGE results for HIC-8 run. M—ladder. Fractions E1, W3 and CIP are 5-fold, 5-fold and 2-fold diluted, respectively. Main fraction is E1. VP1-VP3 proteins are marked by rectangle in E1 5× dill. lane. All fractions were desalted and loaded to the gel either neat or diluted under reducing conditions.

FIG. 345 is a table showing conditions for intermediate polishing on CEX using CIM SO3 column. For the preparative run, the entire elution (E1) from HIC-OH was prepared to match binding conditions and loaded to CEX-SO3 column. The run was a seventh run for CEX conditions (SO3-7).

FIGS. 346A and 346B show a chromatogram from run SO3-7. Entire run—loading phase (FIG. 346A), zoomed elution section (FIG. 346B). Legend: blue line is UV detection at 280 nm, red line is UV detection at 260 nm, brown line is conductivity, dark green line is pressure. No pressure rise during loading. Fractions are noted with brown markers. Main elution is E1.

FIGS. 347A-J are chromatograms based on HPLC analytics—Total method for SO3-7. FIG. 347A—blank (buffer) run; FIG. 347B—Load BF; FIG. 347C—load; FIG. 347D—flow through+wash 1 (FT+W1); FIG. 347E—wash 2 (W2); FIG. 347F—elution (E1); FIG. 347G—wash 3 (W3); FIG. 347H—CIP; FIG. 347I—overlay of fluorescence signal; FIG. 347J—overlay of MALS signal. Legend: Fluorescence (Ex 280 nm, EM 348 nm): green curve, Absorbance at 260 nm: red curve, Absorbance at 280 nm: blue curve, Conductivity (mS/cm): black curve. Main elution (E1) is 5-fold diluted where other fractions are non-diluted. All chromatograms are on the same scale.

FIG. 348 is a table showing recoveries based on ddPCR and HPLC total analytics for preparation run SO3-7. Recoveries for intermediate polishing step CEX-SO3 compared to starting HIC-8 E1 material were 90% and 86% for ddPCR and HPLC Total analytics (MALS), respectively. The discrepancy between two methods was minor. In case of HPLC analytics, mass balance was not 100%. Normalization of two (ddPCR and HPLC Total analytics (MALS) results provided a more accurate value with average 97% recovery of AAV in main fraction.

FIG. 349 shows SDS-PAGE results for SO3-7 run. M—ladder. Fraction E1, is 5-fold, and 10-fold diluted, fractions W3 and CIP are 2-fold diluted. Main fraction is E1. VP1-VP3 proteins are marked by rectangle.

FIG. 350 is a table showing the conditions for empty and full AAV capsids separation on AEX using CIM QA column. During the preparative run, the entire elution (E1) from SO3-7 was diluted to match binding conditions and loaded to AEX-QA column. The run was the third run for AEX conditions (QA-3).

FIGS. 351A and 351B show a chromatogram from run QA-3. Entire run—loading phase (FIG. 351A), zoomed elution section (FIG. 351B). Legend: blue line is UV detection at 280 nm, red line is UV detection at 260 nm, brown line is conductivity. No pressure rise during loading. Fractions are noted with brown markers. Main elution (full capsid AAV) is E3.

FIGS. 352A-H show chromatograms based on HPLC analytics—Empty full method for QA-3. FIG. 352A—SO3-7 E1; FIG. 352B—FT+W; FIG. 352C—E1; FIG. 352D—E2 (empty AAV capsids); FIG. 352E—E3 (full AAV capsids); FIG. 352F—E4 (tail portion of main full peak); FIG. 352G—E5; FIG. 352H—E6, FIG. 352I—CIP, FIG. 352J—overlay of MALS signals. Legend: Legend: Fluorescence (Ex 280 nm, EM 348 nm): green curve, Absorbance at 260 nm: red curve, Absorbance at 280 nm: blue curve, Conductivity (mS/cm): black curve, multi angle light scattering detector (MALS) is pink curve. B, C, D, F and G are on the same scale, A, and E are on 3-fold and 8-fold larger scale respectively. Fractions are 20-fold diluted (picture I) or 10-fold (picture E and H) others are 5-fold diluted. Ratios A260/A280 are presented on the corresponding fractions.

FIG. 353 is a table showing conditions for achieving buffer exchange using dialysis on the QA-3 E3 sample. End volume of sample was 3 mL.

FIGS. 354A-C are tables showing recoveries based on ddPCR and HPLC E/F analysis for preparative run A-3 (FIG. 354A), genomic DSP yield (FIG. 354B), and normalized DSP yield (FIG. 354C).

FIG. 355 is a table showing purity (ratio between empty and full AAV capsids) based on HPLC E/F analytics.

FIG. 356 is a table showing the ratio of full and empty AAV capsids evaluated by TEM in diluted and non-diluted QA-15 and after TFF samples.

FIG. 357 provides micrographs of SO3-7 E1 (top row), QA-3 E3 (middle row) and after dialysis (bottom row) evaluated by TEM. Left: low magnification, right: magnification used for counting.

FIG. 358 shows silver-stained SDS-PAGE results for QA-3 run. M—ladder. Fraction E3 is neat and 5-fold diluted, others beside CIP (2-fold) are neat. Main fraction is E3. AAV8-PD is E3 fraction after dialysis. Biorad TGX 4-20% gel was used, silver staining procedure.

FIGS. 359A and B show HPLC chromatograms—fingerprint method. Overlay of each chromatographic stage is presented. FIG. 359A: overlay of A260 signal. FIG. 359B: overlay of MALS signal, which portrays only larger particles and it is not affected by proteins, and thus, a better resolution of E/F is obtained. Fractions were diluted proportionally to have similar response.

### DETAILED DESCRIPTION

The disclosure provides a method of purifying a recombinant AAV (rAAV) particle from a mammalian host cell culture, comprising the steps of: (a) culturing a plurality of mammalian host cells in a growth media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells; (b) contacting the plurality of transfected mammalian host cells and a virus release solution under conditions suitable for the release of rAAV particles into a harvest media to produce a composition comprising a plurality of rAAV particles, virus release solution and harvest media; (c) purifying the plurality of rAAV particles from the composition of (b) through hydrophobic interaction chromatography (HIC) to produce a HIC eluate comprising the plurality of rAAV particles; (d) purifying the plurality of rAAV viral particles from the HIC eluate of (b) through cation exchange chromatography (CEX) to produce a CEX eluate comprising a plurality of rAAV particles; (e) isolating a plurality of full rAAV particles from the CEX eluate of (d) by anion exchange (AEX) chromatography to produce a AEX eluate comprising a purified and enriched plurality of full rAAV particles; and (f) diafiltering and concentrating the AEX eluate of (e) into a final formulation buffer by tangential flow filtration (TFF) to produce a final composition comprising a purified and enriched plurality of full rAAV particles and the final formulation buffer.

The disclosure further related to methods of producing a recombinant AAV (rAAV) particle, comprising the steps of: (a) transfecting a plurality of mammalian host cells with a plasmid vector comprising an exogenous sequence, a helper plasmid vector, and a plasmid vector comprising a sequence encoding a viral Rep protein and a viral Cap protein to produce a plurality of transfected mammalian host cells, wherein the cells are transfected using PEI as a transfection reagent, and wherein the cells are contacted with the PEI and the vectors at specified ratios of the plasmid vectors.

### AAV-RPGR

The disclosure provides a composition manufactured using the methods of the disclosure. In some embodiments, the composition comprises (a) between 0.5×10¹¹ vector genomes (vg)/mL and 1×10¹³ vg/mL of replication-defective and recombinant adeno-associated virus (rAAV), (b) less than 50% empty capsids; and (c) a plurality of functional vg/mL, wherein each of functional vector genomes is capable of expressing an RPGR^{ORF15} sequence in a cell following transduction. In some embodiments, the composition comprises (a) between 0.5×10¹¹ vector genomes (vg)/mL and 1×10¹³ vg/mL of replication-defective and recombinant adeno-associated virus (rAAV), (b) less than 30% empty capsids; and (c) a plurality of functional vg/mL, wherein each of functional vector genomes is capable of expressing an RPGR^{ORF15} sequence in a cell following transduction. In some embodiments, the composition comprises (a) between 0.5×10¹¹ vector genomes (vg)/mL and 1×10¹³ vg/mL of replication-defective and recombinant adeno-associated virus (rAAV), (b) less than 99%, 97%, 95%, 90%, 85%, 80%, 75%, 70%, 65%, 60%, 55%, 50%, 45%, 40%, 35%, 30%, 25%, 20%, 15%, 10%, 5%, 2%, 1%, or any percentage in between of empty capsids; and (c) a plurality of functional vg/mL, wherein each of functional vector genomes is capable of expressing an RPGR^{ORF15} sequence in a cell following transduction. In some embodiments, following transduction of a cell with a composition of the disclosure, the RPGR^{ORF15} sequence encodes a RPGR^{ORF15} protein. In some embodiments, the protein encoded by the RPGR^{ORF15} sequence has an activity level equal to or greater than an activity level of an RPGR^{ORF15} encoded by a corresponding sequence of a nontransduced cell. In some embodiments, the exogenous RPGR^{ORF15} sequence and the corresponding endogenous RPGR^{ORF15} sequence are identical. In some embodiments, the exogenous RPGR^{ORF15} sequence and the corresponding endogenous RPGR^{ORF15} sequence are not identical. In some embodiments, the exogenous RPGR^{ORF15} sequence and the corresponding endogenous RPGR^{ORF15} sequence have at least 70%, 75%, 80%, 85%, 90%, 95%, 97%, 99% or any percentage in between of identity.

In some embodiments of the compositions of the disclosure, the composition comprises (a) between 0.5×10¹¹ vg/mL and 1×10¹³ vg/mL, inclusive of the endpoints, (b) at least 70% full capsids and (c) a plurality of functional vg/mL, wherein each of functional vector genomes is capable of expressing an RPGR^{ORF15} sequence in a cell following transduction. In some embodiments, the composition comprises (a) between 0.5×10¹¹ vg/mL and 1×10¹³ vg/mL, inclusive of the endpoints, (b) at least 1%, 2%, 5%, 10%, 15%, 20%, 25%, 30%, 35%, 40%, 45%, 50%, 55%, 60%, 65%, 70%, 75%, 80%, 85%, 90%, 95%, 97%, 99%, 100%, or any percentage in between of full capsids and (c) a plurality of functional vg/mL, wherein each of functional vector genomes is capable of expressing an RPGR^{ORF15} sequence in a cell following transduction. In some embodiments, the composition comprises 0.5×10¹¹ vg/mL. In some embodiments, the composition comprises 1×10¹² vg/mL.

Compositions of the disclosure comprise a therapeutic RPGR^{ORF15} construct suitable for systemic or local administration to a mammal, and preferable, to a human. Exemplary RPGR^{ORF15} constructs of the disclosure comprise a sequence encoding a RPGR^{ORF15} or a portion thereof. Preferably, RPGR^{ORF15} constructs of the disclosure comprise a sequence encoding a human RPGR^{ORF15} or a portion thereof. Exemplary RPGR^{ORF15} constructs of the disclosure may further comprise one or more sequence(s) encoding regulatory elements to enable or to enhance expression of the gene or a portion thereof. Exemplary regulatory elements include, but are not limited to, promoters, introns, enhancer elements, response elements (including post-transcriptional response elements or post-transcriptional regulatory elements), polyadenosine (polyA) sequences, and a gene fragment to facilitate efficient termination of transcription (including a β-globin gene fragment and a rabbit β-globin gene fragment).

In some embodiments of the compositions of the disclosure, the RPGR^{ORF15} construct comprises a human gene or a portion thereof corresponding to a human Retinitis Pigmentosa GTPase Regulator (RPGR) protein or a portion thereof. Human RPGR comprises multiple spliced isoforms. Isoform ORF15 RPGR (RPGR^{ORF15}) localizes to the photoreceptors. In some embodiments, the RPGR protein is RPGR^{ORF15}. In some embodiments, the RPGR^{ORF15} construct comprises a human gene or a portion thereof comprising a codon-optimized sequence. In some embodiments, the sequence is codon-optimized for expression in mammals. In some embodiments, the sequence is codon-optimized for expression in humans.

In some embodiments of the compositions of the disclosure, the AAV-RPGR^{ORF15} product consists of a purified recombinant serotype 2 (rAAV) encoding the cDNA of RPGR^{ORF15}. In some embodiments, each 20 nm AAV virion contains a single stranded DNA insert sequence comprising: an AAV2 5′ inverted terminal repeat (ITR), a 199 bp GRK1 promoter, a 3459 bp human RPGR^{ORF15} cDNA, a 270 bp Bovine growth hormone polyadenylation sequence (BGH-polyA), and an AAV2 3′ ITR, as well a short cloning sequences flanking the elements.

In some embodiments, the RPGR^{ORF15} construct comprises a sequence encoding RPGR^{ORF15} In some embodiments, the sequence encoding the RPGR^{ORF15} is a human RPGR^{ORF15} sequence. In some embodiments, the sequence encoding RPGR^{ORF15} comprises a nucleotide sequence encoding an amino acid sequence that has at least 80% identity, at least 90% identity, at least 95% identity, at least 97% identity, at least 99% identity or is identical to the amino acid sequence of:

| (SEQ ID NO: 78) | |
|---|---|
| 1 | MREPEELMPD SGAVFTFGKS KFAENNPGKF WFKNDVPVHL SCGDEHSAVV TGNNKLYMFG |
| 61 | SNNWGQLGLG SKSAISKPTC VKALKPEKVK LAACGRNHTL VSTEGGNVYA TGGNNEGQLG |
| 121 | LGDTEERNTF HVISFFTSEH KIKQLSAGSN TSAALTEDGR LFMWGDNSEG QIGLKNVSNV |
| 181 | CVPQQVTIGK PVSWISCGYY HSAFVTTDGE LYVFGEPENG KLGLPNQLLG NHRTPQLVSE |
| 241 | IPEKVIQVAC GGEHTVVLTE NAVYTFGLGQ FGQLGLGTFL FETSEPKVIE NIRDQTISYI |
| 301 | SCGENHTALI TDIGLMYTFG DGRHGKLGLG LENFTNHFIP TLCSNFLRFI VKLVACGGCH |
| 361 | MVVFAAPHRG VAKEIEFDEI NDTCLSVATF LPYSSLTSGN VLQRTLSARM RRRERERSPD |
| 421 | SFSMRRTLPP IEGTLGLSAC FLPNSVFPRC SERNLQESVL SEQDLMQPEE PDYLLDEMTK |
| 481 | EAEIDNSSTV ESLGETTDIL NMTHIMSLNS NEKSLKLSPV QKQKKQQTIG ELTQDTALTE |
| 541 | NDDSDEYEEM SEMKEGKACK QHVSQGIFMT QPATTIEAFS DEEVEIPEEK EGAEDSKGNG |
| 601 | IEEQEVEANE ENVKVHGGRK EKTEILSDDL TDKAEVSEGK AKSVGEAEDG PEGRGDGTCE |
| 661 | EGSSGAEHWQ DEEREKGEKD KGRGEMERPG EGEKELAEKE EWKKRDGEEQ EQKEREQGHQ |
| 721 | KERNQEMEEG GEEEHGEGEE EEGDREEEEE KEGEGKEEGE GEEVEGEREK EEGERKKEER |
| 781 | AGKEEKGEEE GDQGEGEEEE TEGRGEEKEE GGEVEGGEVE EGKGEREEEE EEGEGEEEEG |
| 841 | EGEEEEGEGE EEEGEGKGEE EGEEGEGEEE GEEGEGEGEE EEGEGEGEEE GEGEGEEEEG |
| 901 | EGEGEEEGEG EGEEEEGEGK GEEEGEEGEG EGEEEEGEGE GEDGEGEGEE EEGEWEGEEE |
| 961 | EGEGEGEEEG EGEGEEGEGE GEEEEGEGEG EEEEGEEEGE EEGEGEEEGE GEGEEEEEGE |
| 1021 | VEGEVEGEEG EGEGEEEEGE EEGEEREKEG EGEENRRNRE EEEEEEGKYQ ETGEEENERQ |
| 1081 | DGEEYKKVSK IKGSVKYGKH KTYQKKSVTN TQGNGKEQRS KMPVQSKRLL KNGPSGSKKF |
| 1141 | WNNVLPHYLE LK. |

In some embodiments, the sequence encoding RPGR^{ORF15} comprises a wild type nucleotide sequence. In some embodiments, the sequence encoding RPGR^{ORF15} comprises a nucleotide sequence that has at least 70%, at least 75%, at least 80%, at least 85%, at least 90%, at least 95%, at least 97%, at least 99% or any percentage in between of identity to the nucleotide sequence of:

| (SEQ ID NO: 79) | |
|---|---|
| 1 | atgagggagc cggaagagct gatgcccgat tcgggtgctg tgtttacatt tgggaaaagt |
| 61 | aaatttgctg aaaataatcc cggtaaattc tggtttaaaa atgatgtccc tgtacatctt |
| 121 | tcatgtggag atgaacattc tgctgttgtt accggaaata ataaacttta catgtttggc |
| 181 | agtaacaact ggggtcagtt aggattagga tcaaagtcag ccatcagcaa gccaacatgt |
| 241 | gtcaaagctc taaaacctga aaaagtgaaa ttagctgcct gtggaaggaa ccacaccctg |
| 301 | gtgtcaacag aaggaggcaa tgtatatgca actggtggaa ataatgaagg acagttgggg |
| 361 | cttggtgaca ccgaagaaag aaacactttt catgtaatta gcttttttac atccgagcat |
| 421 | aagattaagc agctgtctgc tggatctaat acttcagctg ccctaactga ggatggaaga |
| 481 | ctttttatgt ggggtgacaa ttccgaaggg caaattggtt taaaaaatgt aagtaatgtc |
| 541 | tgtgtccctc agcaagtgac cattgggaaa cctgtctcct ggatctcttg tggatattac |
| 601 | cattcagctt ttgtaacaac agatggtgag ctatatgtgt ttggagaacc tgagaatggg |
| 661 | aagttaggtc ttcccaatca gctcctgggc aatcacagaa caccccagct ggtgtctgaa |
| 721 | attccggaga aggtgatcca agtagcctgt ggtggagagc atactgtggt tctcacggag |
| 781 | aatgctgtgt atacctttgg gctgggacaa tttggtcagc tgggtcttgg cacttttctt |
| 841 | tttgaaactt cagaacccaa agtcattgag aatattaggg atcaaacaat aagttatatt |
| 901 | tcttgtggag aaaatcacac agctttgata acagatatcg gccttatgta tacttttgga |
| 961 | gatggtcgcc acggaaaatt aggacttgga ctggagaatt ttaccaatca cttcattcct |
| 1021 | actttgtgct ctaatttttt gaggtttata gttaaattgg ttgcttgtgg tggatgtcac |
| 1081 | atggtagttt ttgctgctcc tcatcgtggt gtggcaaaag aaattgaatt cgatgaaata |
| 1141 | aatgatactt gcttatctgt ggcgactttt ctgccgtata gcagtttaac ctcaggaaat |
| 1201 | gtactgcaga ggactctatc agcacgtatg cggcgaagag agagggagag gtctccagat |
| 1261 | tctttttcaa tgaggagaac actacctcca atagaaggga ctcttggcct ttctgcttgt |
| 1321 | tttctcccca attcagtctt tccacgatgt tctgagagaa acctccaaga gagtgtctta |
| 1381 | tctgaacagg acctcatgca gccagaggaa ccagattatt tgctagatga aatgaccaaa |
| 1441 | gaagcagaga tagataattc ttcaactgta gaaagccttg gagaaactac tgatatctta |
| 1501 | aacatgacac acatcatgag cctgaattcc aatgaaaagt cattaaaatt atcaccagtt |
| 1561 | cagaaacaaa agaaacaaca aacaattggg gaactgacgc aggatacagc tcttactgaa |
| 1621 | aacgatgata gtgatgaata tgaagaaatg tcagaaatga aagaagggaa agcatgtaaa |
| 1681 | caacatgtgt cacaagggat tttcatgacg cagccagcta cgactatcga agcattttca |
| 1741 | gatgaggaag tagagatccc agaggagaag gaaggagcag aggattcaaa aggaaatgga |
| 1801 | atagaggagc aagaggtaga agcaaatgag gaaaatgtga aggtgcatgg aggaagaaag |
| 1861 | gagaaaacag agatcctatc agatgacctt acagacaaag cagaggtgag tgaaggcaag |
| 1921 | gcaaaatcag tgggagaagc agaggatggg cctgaaggta gaggggatgg aacctgtgag |
| 1981 | gaaggtagtt caggagcaga acactggcaa gatgaggaga gggagaaggg ggagaaagac |
| 2041 | aagggtagag gagaaatgga gaggccagga gagggagaga aggaactagc agagaaggaa |
| 2101 | gaatggaaga agagggatgg ggaagagcag gagcaaaagg agagggagca gggccatcag |
| 2161 | aaggaaagaa accaagagat ggaggaggga ggggaggagg agcatggaga aggagaagaa |
| 2221 | gaggagggag acagagaaga ggaagaagag aaggagggag aagggaaaga ggaaggagaa |
| 2281 | ggggaagaag tggagggaga acgtgaaaag gaggaaggag agaggaaaaa ggaggaaaga |
| 2341 | gcggggaagg aggagaaagg agaggaagaa ggagaccaag gagaggggga agaggaggaa |
| 2401 | acagagggga gaggggagga aaaagaggag ggaggggaag tagagggagg ggaagtagag |
| 2461 | gaggggaaag gagagaggga agaggaagag gaggagggtg agggggaaga ggaggaaggg |
| 2521 | gagggggaag aggaggaagg ggagggggaa gaggaggaag gagaagggaa aggggaggaa |
| 2581 | gaaggggaag aaggagaagg ggaggaagaa ggggaggaag gagaagggga gggggaagag |
| 2641 | gaggaaggag aaggggaggg agaagaggaa ggagaagggg agggagaaga ggaggaagga |
| 2701 | gaaggggagg gagaagagga aggagaaggg gagggagaag aggaggaagg agaagggaaa |
| 2761 | ggggaggagg aaggagagga aggagaaggg gagggggaag aggaggaagg agaaggggaa |
| 2821 | ggggaggatg gagaagggga gggggaagag gaggaaggag aatgggaggg ggaagaggag |
| 2881 | gaaggagaag gggaggggga agaggaagga gaaggggaag gggaggaagg agaaggggag |
| 2941 | ggggaagagg aggaaggaga aggggagggg gaagaggagg aaggggaaga agaaggggag |
| 3001 | gaagaaggag agggagagga agaaggggag ggagaagggg aggaagaaga ggaaggggaa |
| 3061 | gtggaagggg aggtggaagg ggaggaagga gagggggaag gagaggaaga ggaaggagag |
| 3121 | gaggaaggag aagaaaggga aaaggagggg gaaggagaag aaaacaggag gaacagagaa |
| 3181 | gaggaggagg aagaagaggg gaagtatcag gagacaggcg aagaagagaa tgaaaggcag |
| 3241 | gatggagagg agtacaaaaa agtgagcaaa ataaaaggat ctgtgaaata tggcaaacat |
| 3301 | aaaacatatc aaaaaaagtc agttactaac acacagggaa atgggaaaga gcagaggtcc |
| 3361 | aaaatgccag tccagtcaaa acgactttta aaaaacgggc catcaggttc caaaaagttc |
| 3421 | tggaataatg tattaccaca ttacttggaa ttgaagtaa |

In some embodiments, the sequence encoding RPGR^{ORF15} comprises a codon optimized nucleotide sequence. RPGR^{ORF15} contains a highly repetitive purine-rich region at the 3′-end and a splice site immediately upstream, which can create significant challenges in cloning an AAV.RPGR vector. In some embodiments, codon optimization can be used to disable the endogenous splice site and stabilize the purine-rich sequence in the RPGR^{ORF15} transcript without altering the amino acid sequence of the RPGR^{ORF15} protein. In some embodiments, post-translation modifications such as glutamylation of RPGR protein are preserved following codon-optimization. In some embodiments, the RPGR^{ORF15} nucleotide sequence is codon optimized for expression in a mammal. In some embodiments, the RPGR^{ORF15} nucleotide sequence is codon optimized for expression in a human.

In some embodiments, the codon optimized 3459 bp human RPGR^{ORF15} cDNA comprises a nucleotide sequence that has at least 70% identity, at least 75% identity, at least 80% identity, at least 85% identity, at least 90% identity, at least 95% identity, at least 97% identity, at least 99% identity or any percentage in between of identity to the nucleotide sequence of:

| (SEQ ID NO: 80) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3061 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa |

In some embodiments, the codon optimized 3459 bp human RPGR^{ORF15} cDNA comprises or consists of the nucleotide sequence of:

| (SEQ ID NO: 81) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3061 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa |

In some embodiments of the compositions of the disclosure, the RPGR^{ORF15} construct comprises a promoter. In some embodiments, the promoter comprises a rhodopsin kinase promoter. In some embodiments, the rhodopsin kinase promoter is isolated or derived from the promoter of the G protein-coupled receptor kinase 1 (GRK1) gene. In some embodiments, the promoter is a GRK1 promoter. In some embodiments, the sequence encoding the GRK1 promoter comprises a sequence having at least 80% identity, at least 90% identity, at least 95% identity, at least 97% identity or at least 99% identity to:

| (SEQ ID NO: 82) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg |

In some embodiments, the GRK1 promoter comprises or consists of:

| (SEQ ID NO: 82) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg |

In some embodiments of the compositions of the disclosure, the RPGR^{ORF15} construct comprises a polyadenylation signal. In some embodiments, the sequence encoding the polyA signal comprises a polyA signal isolated or derived from a bovine growth hormone (BGH) polyA signal. In some embodiments, the BGH polyA signal comprises a nucleotide sequence that has at least 80% identity, at least 97% identity or 100% identity to the nucleotide sequence of:

| (SEQ ID NO: 83) | |
|---|---|
| 1 | cgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg |

In some embodiments, the sequence encoding the BGH polyA comprises or consists of the nucleotide sequence of:

| (SEQ ID NO: 83) | |
|---|---|
| 1 | cgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg |

In some embodiments of the compositions of the disclosure, the RPGR^{ORF15} construct further comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2) In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type AAV2 sequence. In some embodiments, the variation comprises a substitution, an insertion, a deletion, an inversion, or a transposition. In some embodiments, the variation comprises a truncation or an elongation of a wild type or a variant sequence.

In some embodiments of the compositions of the disclosure, an AAV comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2) In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type AAV2 sequence. In some embodiments, the variation comprises a substitution, an insertion, a deletion, an inversion, or a transposition. In some embodiments, the variation comprises a truncation or an elongation of a wild type or a variant sequence.

In some embodiments of the compositions of the disclosure, an AAV comprises a viral sequence essential for formation of a replication-deficient AAV. In some embodiments, the viral sequence is isolated or derived from an AAV of the same serotype as one or both of the sequence encoding the 5′ITR or the sequence encoding the 3′ITR. In some embodiments, the viral sequence, the sequence encoding the 5′ITR or the sequence encoding the 3′ITR are isolated or derived from an AAV2.

In some embodiments of the compositions of the disclosure, an AAV comprises a viral sequence essential for formation of a replication-deficient AAV, a sequence encoding the 5′ITR and a sequence encoding the 3′ITR, but does not comprise any other sequence isolated or derived from an AAV. In some embodiments, the AAV is a recombinant AAV (rAAV), comprising a viral sequence essential for formation of a replication-deficient AAV, a sequence encoding the 5′ITR, a sequence encoding the 3′ITR, and a sequence encoding an RPGR^{ORF15} construct of the disclosure.

In some embodiments, a plasmid DNA used to create the rAAV in a host cell comprises a selection marker. Exemplary selection markers include, but are not limited to, antibiotic resistance genes. Exemplary antibiotic resistance genes include, but are not limited to, ampicillin and kanamycin. Exemplary selection markers include, but are not limited to, drug or small molecule resistance genes. Exemplary selection markers include, but are not limited to, dapD and a repressible operator including but not limited to a lacO/P construct controlling or suppressing dapD expression, wherein plasmid selection is performed by administering or contacting a transformed cell with a plasmid capable of operator repressor titration (ORT). Exemplary selection markers include, but are not limited to, a ccd selection gene. In some embodiments, the ccd selection gene comprises a sequence encoding a ccdA selection gene that rescues a host cell line engineered to express a toxic ccdB gene. Exemplary selection markers include, but are not limited to, sacB, wherein an RNA is administered or contacted to a host cell to suppress expression of the sacB gene in sucrose media. Exemplary selection markers include, but are not limited to, a segregational killing mechanism such as the parAB+ locus composed of Hok (a host killing gene) and Sok (suppression of killing).

### AAV-RPGR Construct Structure

The AAV-RPGR^{ORF15} construct product consists of a purified recombinant serotype 2 adeno-associated viral vector (rAAV) encoding the cDNA encoding a therapeutic construct.

In some embodiments, the AAV-RPGR^{ORF15} construct comprises one or more of a sequence encoding a 5′ ITR, a sequence encoding a 3′ ITR and a sequence encoding a capsid protein that is isolated and/or derived from a serotype 8 adeno-associated viral vector (AAV8). In some embodiments, the AAV-RPGR^{ORF15} construct comprises a sequence encoding a 5′ ITR, a sequence encoding a 3′ ITR and a sequence encoding a capsid protein that is isolated and/or derived from a serotype 8 adeno-associated viral vector (AAV8). In some embodiments, the AAV-RPGR^{ORF15} construct comprises a truncated sequence encoding a 5′ ITR and a sequence encoding a 3′ ITR that is isolated and/or derived from a serotype 2 adeno-associated viral vector (AAV2) and a sequence encoding a capsid protein that is isolated and/or derived from a serotype 8 adeno-associated viral vector (AAV8). In some embodiments, the AAV-Construct comprises wild type AAV2 ITRs (a wild type 5′ ITR and a wild type 3′ ITR).

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding RPGR^{ORF15}, and (d) a 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding RPGR^{ORF15}, (c) a polyadenylation signal, and (d) a bp 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding RPGR^{ORF15}, (d) a post-transcriptional regulatory element (PRE), (e) a polyadenylation sequence (polyA), and (f) a 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter, optionally, a 199 bp GRK1 promoter, (c) a cDNA encoding RPGR^{ORF15}, (d) a 270 bp Bovine growth hormone polyadenylation sequence (BGH-polyA), and (e) a 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter, optionally, a 199 bp GRK1 promoter, (c) a cDNA encoding RPGR^{ORF15}, (d) a 270 bp Bovine growth hormone polyadenylation sequence (BGH-polyA), and (e) a 3′ ITR.

AAVs or RPGR^{ORF15} constructs of the disclosure may comprise a sequence encoding a promoter capable of expression in a mammalian cell. Preferably, AAVs or RPGR^{ORF15} constructs of the disclosure may comprise a sequence encoding a promoter capable of expression in a human cell. Exemplary promoters of the disclosure include, but are not limited to, constitutively active promoters, cell-type specific promoters, viral promoters, mammalian promoters, and hybrid or recombinant promoters. In some embodiments of the compositions of the disclosure, the therapeutic Construct of an AAV-Construct is under the control of a G protein-coupled receptor kinase 1 (GRK1) promoter.

AAVs or RPGR^{ORF15} constructs of the disclosure may comprise a polyadenosine (polyA) sequence. Exemplary polyA sequences of the disclosure include, but are not limited to, a bovine growth hormone polyadenylation (BGH-polyA) sequence. The BGH-polyA sequence is used to enhance gene expression and has been shown to yield three times higher expression levels than other polyA sequences such as SV40 and human collagen polyA. This increased expression is largely independent of the type of upstream promoter or transgene. Increasing expression levels using a BGH-polyA sequence allows a lower overall dose of AAV or plasmid vector to be injected, which is less likely to generate a host immune response.

In some embodiments of the compositions of the disclosure, the composition comprises a Drug Substance. As used herein, a Drug Substance comprises a rAAV of the disclosure comprising a RPGR^{ORF15} construct of the disclosure.

AAV-ABCA4

The disclosure provides a composition manufactured using the methods of the disclosure. In some embodiments, the composition comprises (a) between 0.5×10¹¹ vector genomes (vg)/mL and 5×10¹³ vg, or between 0.5×10¹¹ vg/mL and 1×10¹³ vg/mL, of replication-defective, recombinant adeno-associated virus (rAAV) upstream or downstream vector, respectively, (b) less than 50% empty capsids; and (c) a plurality of functional vg/mL, wherein a pair of upstream and downstream functional vector genomes is capable of expressing an ABCA4 sequence in a cell following transduction. In some embodiments, the composition comprises (a) between 0.5×10¹¹ vector genomes (vg)/mL and 5×10¹³ vg/mL, or between 0.5×10¹¹ vg/mL and 1×10¹³ vg/mL, of replication-defective, recombinant adeno-associated virus (rAAV) upstream or downstream vector, respectively, (b) less than 30% empty capsids; and (c) a plurality of functional vg/mL, wherein a pair of upstream and downstream functional vector genomes is capable of expressing an ABCA4 sequence in a cell following transduction. In some embodiments, the composition comprises (a) between 0.5×10¹¹ vector genomes (vg)/mL and 5×10¹³ vg/mL, or between 0.5×10¹¹ vg/mL and 1×10¹³ vg/mL, of replication-defective, recombinant adeno-associated virus (rAAV) upstream or downstream vector, respectively, (b) less than 99%, 97%, 95%, 90%, 85%, 80%, 75%, 70%, 65%, 60%, 55%, 50%, 45%, 40%, 35%, 30%, 25%, 20%, 15%, 10%, 5%, 2%, 1%, or any percentage in between of empty capsids; and (c) a plurality of functional vg/mL, wherein a pair of upstream and downstream functional vector genomes is capable of expressing an ABCA4 sequence in a cell following transduction. In some embodiments, following transduction of a cell with a composition of the disclosure, the ABCA4 sequence encodes an ABCA4 protein. In some embodiments, the protein encoded by the ABCA4 sequence has an activity level equal to or greater than an activity level of an ABCA4 encoded by a corresponding sequence of a nontransduced cell. In some embodiments, the exogenous ABCA4 sequence and the corresponding endogenous ABCA4 sequence are identical. In some embodiments, the exogenous ABCA4 sequence and the corresponding endogenous ABCA4 sequence are not identical. In some embodiments, the exogenous ABCA4 sequence and the corresponding endogenous ABCA4 sequence have at least 70%, 75%, 80%, 85%, 90%, 95%, 97%, 99% or any percentage in between of identity.

In some embodiments of the compositions of the disclosure, the composition comprises (a) between 0.5×10¹¹ vg/mL and 5×10¹³ vg/mL, or between 0.5×10¹¹ vg/mL and 1×10¹³ vg/mL, inclusive of the endpoints, of upstream or downstream vector, respectively (b) at least 70% full capsids and (c) a plurality of functional vg/mL, wherein a pair of upstream and downstream functional vector genomes is capable of expressing an ABCA4 sequence in a cell following transduction. In some embodiments, the composition comprises (a) between 0.5×10¹¹ vg/mL and 5×10¹³ vg/mL, or between 0.5×10¹¹ vg/mL and 1×10¹³ vg/mL, inclusive of the endpoints, of upstream or downstream vector, respectively (b) at least 1%, 2%, 5%, 10%, 15%, 20%, 25%, 30%, 35%, 40%, 45%, 50%, 55%, 60%, 65%, 70%, 75%, 80%, 85%, 90%, 95%, 97%, 99%, 100%, or any percentage in between of full capsids and (c) a plurality of functional vg/mL, wherein a pair of upstream and downstream functional vector genomes is capable of expressing an ABCA4 sequence in a cell following transduction.

Compositions of the disclosure comprise a therapeutic ABCA4 construct suitable for systemic or local administration to a mammal, and preferable, to a human. Exemplary ABCA4 constructs of the disclosure comprise a sequence encoding an ABCA4 or a portion thereof. Preferably, ABCA4 constructs of the disclosure comprise a sequence encoding a human ABCA4 or a portion thereof. Exemplary ABCA4 constructs of the disclosure may further comprise one or more sequence(s) encoding regulatory elements to enable or to enhance expression of the gene or a portion thereof. Exemplary regulatory elements include, but are not limited to, promoters, introns, enhancer elements, response elements (including post-transcriptional response elements or post-transcriptional regulatory elements), polyadenosine (polyA) sequences, and a gene fragment to facilitate efficient termination of transcription (including a β-globin gene fragment and a rabbit β-globin gene fragment).

In some embodiments of the compositions of the disclosure, the ABCA4 construct comprises a human gene (or variant thereof) or a portion thereof corresponding to a human ATP-Binding Cassette, Subfamily A, Member 4 (ABCA4) protein or a portion thereof. Human ABCA4 localizes to the photoreceptors. In some embodiments, the ABCA4 construct comprises a human gene or a portion thereof comprising a wild type or codon-optimized sequence. In some embodiments, the sequence is codon-optimized for expression in mammals. In some embodiments, the sequence is codon-optimized for expression in humans. In some embodiments, an upstream ABCA4 construct comprises a 5′ portion of a human ABCA4 gene and a downstream ABCA4 construct comprises a 3′ portion of a human ABCA4 gene. In some embodiments, the 5′ portion of a human ABCA4 gene and the 3′ portion of a human ABCA4 gene each comprise a sequence that “overlaps” with the other, meaning that the overlapping sequence forms a duplex in which the sequence of the overlapping portion of the 5′ portion of a human ABCA4 gene is complementary to the sequence of the overlapping portion of the 3′ portion of a human ABCA4 gene. In some embodiments the sequence of the overlapping portion of the 5′ portion of a human ABCA4 gene comprises or consists of at least 5, 10, 15, 20, 25, 30, 35, 40, 45, 50, 55, 60, 65, 70, 75, 80, 85, 90, 95, 100, 150, 200, 250, 300, 350, 400, 450, 500 or any number of nucleotides in between. In some embodiments the sequence of the overlapping portion of the 5′ portion of a human ABCA4 gene comprises or consists of 20 nucleotides. In some embodiments the sequence of the overlapping portion of the 3′ portion of a human ABCA4 gene comprises or consists of at least 5, 10, 15, 20, 25, 30, 35, 40, 45, 50, 55, 60, 65, 70, 75, 80, 85, 90, 95, 100, 150, 200, 250, 300, 350, 400, 450, 500 or any number of nucleotides in between. In some embodiments the sequence of the overlapping portion of the 3′ portion of a human ABCA4 gene comprises or consists of 20 nucleotides.

In some embodiments of the compositions of the disclosure, the AAV-ABCA4 product comprises or consists of a purified recombinant serotype 8 (rAAV8) encoding a cDNA of ABCA4. In some embodiments, the AAV-ABCA4 product comprises a purified mutant rAAV8 capsid protein where the mutant rAAV8 comprises a substitution of a Phenylalanine for a Tyrosine at amino acid position 733 (AAV8 Y773F mutant). In some embodiments, an AAV-ABCA4 upstream product comprises or consists of a purified recombinant serotype 8 (rAAV8) encoding a cDNA of a 5′ portion of ABCA4. In some embodiments, an AAV-ABCA4 downstream product comprises or consists of a purified recombinant serotype 8 (rAAV8) encoding a cDNA of a 3′ portion of ABCA4. In some embodiments, the ABCA4 or the portion thereof is a human ABCA4.

In some embodiments of an AAV-ABCA4 product of the disclosure, each 20 nm AAV virion contains a single stranded DNA insert sequence comprising: an AAV2 5′ inverted terminal repeat (ITR), an ABCA4 cDNA and an AAV2 3′ ITR, as well a short cloning sequences flanking the elements.

In some embodiments of an AAV-ABCA4 upstream product of the disclosure, each 20 nm AAV virion contains a single stranded DNA insert sequence comprising: an AAV2 5′ inverted terminal repeat (ITR), a promoter, an ABCA4 cDNA and an AAV2 3′ ITR, as well a short cloning sequences flanking the elements. In some embodiments, the ABCA4 cDNA comprises a sequence encoding a 5′ portion of a human ABCA4 gene. In some embodiments, the promoter comprises a GRK1 promoter. In some embodiments, the promoter comprises a chicken beta-actin (CBA) promoter alone or in combination with one or more of a cytomegalovirus (CMV) enhancer and a rabbit beta-Globin (RBG) splice acceptor site. In some embodiments, the promoter comprises a chicken beta-actin (CBA) promoter, a CMV enhancer and a RBG splice acceptor site, otherwise referred to herein as a “CAG” promoter. In some embodiments, the each 20 nm AAV virion contains a single stranded DNA insert sequence further comprising a sequence encoding an intron and/or a sequence encoding an exon.

In some embodiments of an AAV-ABCA4 downstream product of the disclosure, each 20 nm AAV virion contains a single stranded DNA insert sequence comprising: an AAV2 5′ inverted terminal repeat (ITR), an ABCA4 cDNA and an AAV2 3′ ITR, as well a short cloning sequences flanking the elements. In some embodiments, the ABCA4 cDNA comprises a sequence encoding a 3′ portion of a human ABCA4 gene. In some embodiments, the each 20 nm AAV virion contains a single stranded DNA insert sequence further comprising a sequence encoding a posttranslational regulatory element (PRE). In some embodiments, the each 20 nm AAV virion contains a single stranded DNA insert sequence further comprising a sequence encoding a Woodchuck PRE (WPRE). In some embodiments, the each 20 nm AAV virion contains a single stranded DNA insert sequence further comprising a sequence encoding a polyadenylation signal. In some embodiments, the each 20 nm AAV virion contains a single stranded DNA insert sequence further comprising a sequence encoding a bovine growth hormone (BGH) polyadenylation signal.

In some embodiments, the ABCA4 construct comprises a sequence encoding a human ABCA4 or a portion thereof. In some embodiments, the sequence encoding ABCA4 comprises a nucleotide sequence or a portion thereof encoding an amino acid sequence that has at least 80% identity, at least 90% identity, at least 95% identity, at least 97% identity, at least 99% identity or is identical to the amino acid sequence of:

| (SEQ ID NO: 40) | |
|---|---|
| 1 | MGFVRQIQLL LWKNWTLRKR QKIRFVVELV WPLSLFLVLI WLRNANPLYS HHECHFPNKA |
| 61 | MPSAGMLPWL QGIFCNVNNP CFQSPTPGES PGIVSNYNNS ILARVYRDFQ ELLMNAPESQ |
| 121 | HLGRIWTELH ILSQFMDTLR THPEPIAGRG IRIRDILKDE ETLTLFLIKN IGLSDSVVYL |
| 181 | LINSqVRPEQ FAHGVPDLAL KDIACSEALL ERFIIFSQRR GAKTVRYALC SLSQGTLQWI |
| 241 | EDTLYANVDF FKLFRVLPTL LDSRSQGINL RSWGGILSDM SPRIQEFIHR PSMQDLLWVT |
| 301 | RPLMQNGGPE TFTKLMGILS DLLCGYPEGG GSRVLSFNWY EDNNYKAFLG IDSTRKDPIY |
| 361 | SYDRRTTSFC NALIQSLESN PLTKIAWRAA KPLUMGKILY TPDSPAARRI LKNANSTFEE |
| 421 | LEHVRKLVKA WEEVGPQIWY FFDNSTQMNM IRDTLGNPTV KDFLNRQLGE EGITAEAILN |
| 481 | FLYKGPRESQ ADDMANFDWR DIFNITDRTL RLVNQYLECL VLDKFESYND ETQLTQRALS |
| 541 | LLEENMFWAG VVFPDMYPWT SSLPPHVKYK IRMDIDVVEK TNKIKDRYWD SGPRADPVED |
| 601 | FRYIWGGFAY LQDMVEQGIT PSQVQAEAPV GIYTQQMPYP CEVDDSFMII LNRCFPIEMV |
| 551 | LAWIYSVSMT VKSIVLEKEL RLKETLKNQG VSNAVIWCTW FIDSFSIMSM SIFLLTIFIM |
| 721 | HGPILHYSDP FILFLFLLAF STATIMLCFL LSTFFSKASL AAACSGVIYF TLYLPHILCF |
| 781 | AWQDRMTAEL KKAVSLLSPV AFGFGTEYLV RFEEQGLGLQ WSNIGNSPTE GDEFSFLLSM |
| 841 | QMMLLDAVVY GLLAWYLDQV FPGDYGTPLP WYFLLQESYW LGGEGCSTRE ERALEKTEPL |
| 901 | TEETEDPEHP EGIHDSFFER EHPGWVPGVC VKNLVKIFEP CGRPAVDRLN ITFYENQITA |
| 961 | FLGHNGAGKT TTLSILTGLL PPTSGTVLVG GRDIETSLDA VRQSLGMCPQ HNILFHHLTV |
| 1021 | AERMLFYAQL KGKSQEEAQL EMEAMLEDTG LHHKRNEEAQ DLSGGMQRKL SVAIAFVGDA |
| 1081 | KVVILDEPTS GVDPYSRRSI WDLLLKYRSG RTIIMSTHHM DEADLLGDRI AIIAQGRLYC |
| 1141 | SGTPLFLKNC FGTGLYLTLV RKMKNIQSQR KGSEGTCSCS SKGESTTCPA HVDDLTPEQV |
| 1201 | LDGDVNELMD VVLHHVPEAK LVECIGQELI FLLPNKNEKH RAYASLFREL EETLADLGLS |
| 1261 | SFGISDTPLE EIFLKVTEDS DSGPLFAGGA QQKRENVNPR HPCLGPREKA GQTPQDSNVC |
| 1321 | SPGAPAAHPE GQPPPEPECP GPQLNTGTQL VLQHVQALLV KREQHTIRSH KDFLAQIVLP |
| 1381 | ATFVFLALML SIVIPPFGEY PALTLHPWIY GQQYTFFSMD EPGSEQFTVL ADVLLNKPGF |
| 1441 | GNRCLKEGWL PEYPCGNSTP WKTPSVSPNI TQLFQKQKWT QVNPSPSCRC STREKLTMIP |
| 1501 | ECPEGAGGLP PPQRTQRSTE ILQDLTDRNI SDFLVKTYPA LIRSSLKSKF WVNEQRYGGI |
| 1561 | SIGGKLPVVP ITGEALVGFL SDLGRIMNVS GGPITREASK EIPDFLKHLE TEDNIKVWFN |
| 1621 | NKGWHALVSF LNVAHNAILR ASLPKDRSPE EYGITVISQP LNLTKEQLSE ITVLTTSVDA |
| 1681 | VVAICVIFSM SFVRASFVLY LIQERVNKSK HLQFISGVSP TTYWVTNFLW DIMNYSVSAG |
| 1741 | LVVGIFIGFQ KKAYTSPENL PALVALLLLY GWAVIPMMYP ASFLFDVPST AYVALSCANL |
| 1801 | FIGINSSAIT FILELFENNR TLLRFNAVLR KLLIVFPHFC LGRGLIDLAL SQAVTDVYAR |
| 1861 | FGEEHSANPF HWDLIGKNLF AMVVEGVVYF LLTLLVQRHF FLSQWIAEPT KEPIVDEDDD |
| 1921 | VAEERQRIIT GGNKTDILRL HELTKIYPGT SSPAVDRLCV GVRPGECFGL LGVNGAGKTT |
| 1981 | TFKMLTGDTT VTSGDATVAG KSILTNISEV HQNMGYCPQF DAIDELLTGR EHLYLYARLR |
| 2041 | GVPAEEIEKV ANWSIKSLGL TVYADCLAGT YSGGNKRKLS TAIALIGCPP LVLLDEPTTG |
| 2101 | MDPQARRMLW NVIVSIIREG RAVVLTSHSM EECEALCTRL AIMVKGAFRC MGTIQELKSK |
| 2161 | FGDGYIVTMK IKSPKDDLLP DLNPVEQFFQ GNFPGSVQRE RHYNMLQFQV SSSSLARIFQ |
| 2221 | LLLSHKDSLL IEEYSVTQTT LDQVFVNFAK QQTESHDLPL HPRAAGABRQ AQD |

In some embodiments, the sequence encoding ABCA4 comprises a wild type nucleotide sequence. In some embodiments, the sequence encoding ABCA4 comprises a nucleotide sequence that has at least 70%, at least 75%, at least 80%, at least 85%, at least 90%, at least 95%, at least 97%, at least 99% or any percentage in between of identity to the nucleotide sequence of:

| (SEQ ID NO: 1) | |
|---|---|
| 1 | AGGACACAGC GTCCGGAGCC AGAGGCGCTC TTAACGGCGT TTATGTCCTT TGCTGTCTGA |
| 61 | GGGGCCTCAG CTCTGACCAA TCTGGTCTTC GTGTGGTCAT TAGCATGGGC TTCGTGAGAC |
| 121 | AGATACAGCT TTTGCTCTGG AAGAACTGGA CCCTGCGGAA AAGGCAAAAG ATTCGCTTTG |
| 181 | TGGTGGAACT CGTGTGGCCT TTATCTTTAT TTCTGGTCTT GATCTGGTTA AGGAATGCCA |
| 241 | ACCCGCTCTA CAGCCATCAT GAATGCCATT TCCCCAACAA GGCGATGCCC TCAGCAGGAA |
| 301 | TGCTGCCGTG GCTCCAGGGG ATCTTCTGCA ATGTGAACAA TCCCTGTTTT CAAAGCCCCA |
| 361 | CCCCAGGAGA ATCTCCTGGA ATTGTGTCAA ACTATAACAA CTCCATCTTG GCAAGGGTAT |
| 421 | ATCGAGATTT TCAAGAACTC CTCATGAATG CACCAGAGAG CCAGCACCTT GGCCGTATTT |
| 481 | GGACAGAGCT ACACATCTTG TCCCAATTCA TGGACACCCT CCGGACTCAC CCGGAGAGAA |
| 541 | TTGCAGGAAG AGGAATACGA ATAAGGGATA TCTTGAAAGA TGAAGAAACA CTGACACTAT |
| 601 | TTCTCATTAA AAACATCGGC CTGTCTGACT CAGTGGTCTA CCTTCTGATC AACTCTCAAG |
| 661 | TCCGTCCAGA GCAGTTCGCT CATGGAGTCC CGGACCTGGC GCTGAAGGAC ATCGCCTGCA |
| 721 | GCGAGGCCCT CCTGGAGCGC TTCATCATCT TCAGCCAGAG ACGCGGGGCA AAGACGGTGC |
| 781 | GCTATGCCCT GTGCTCCCTC TCCCAGGGCA CCCTACAGTG GATAGAAGAC ACTCTGTATG |
| 841 | CCAACGTGGA CTTCTTCAAG CTCTTCCGTG TGCTTCCCAC ACTCCTAGAC AGCCGTTCTC |
| 901 | AAGGTATCAA TCTGAGATCT TGGGGAGGAA TATTATCTGA TATGTCACCA AGAATTCAAG |
| 961 | AGTTTATCCA TCGGCCGAGT ATGCAGGACT TGCTGTGGGT GACCAGGCCC CTCATGCAGA |
| 1021 | ATGGTGGTCC AGAGACCTTT ACAAAGCTGA TGGGCATCCT GTCTGACCTC CTGTGTGGCT |
| 1081 | ACCCCGAGGG AGGTGGCTCT CGGGTGCTCT CCTTCAACTG GTATGAAGAC AATAACTATA |
| 1141 | AGGCCTTTCT GGGGATTGAC TCCACAAGGA AGGATCCTAT CTATTCTTAT GACAGAAGAA |
| 1201 | CAACATCCTT TTGTAATGCA TTGATCCAGA GCCTGGAGTC AAATCCTTTA ACCAAAATCG |
| 1261 | CTTGGAGGGC GGCAAAGCCT TTGCTGATGG GAAAAATCCT GTACACTCCT GATTCACCTG |
| 1321 | CAGCACGAAG GATACTGAAG AATGCCAACT CAACTTTTGA AGAACTGGAA CACGTTAGGA |
| 1381 | AGTTGGTCAA AGCCTGGGAA GAAGTAGGGC CCCAGATCTG GTACTTCTTT GACAACAGCA |
| 1441 | CACAGATGAA CATGATCAGA GATACCCTGG GGAACCCAAC AGTAAAAGAC TTTTTGAATA |
| 1501 | GGCAGCTTGG TGAAGAAGGT ATTACTGCTG AAGCCATCCT AAACTTCCTC TACAAGGGCC |
| 1561 | CTCGGGAAAG CCAGGCTGAC GACATGGCCA ACTTCGACTG GAGGGACATA TTTAACATCA |
| 1621 | CTGATCGCAC CCTCCGCCTG GTCAATCAAT ACCTGGAGTG CTTGGTCCTG GATAAGTTTG |
| 1681 | AAAGCTACAA TGATGAAACT CAGCTCACCC AACGTGCCCT CTCTCTACTG GAGGAAAACA |
| 1741 | TGTTCTGGGC CGGAGTGGTA TTCCCTGACA TGTATCCCTG GACCAGCTCT CTACCACCCC |
| 1801 | ACGTGAAGTA TAAGATCCGA ATGGACATAG ACGTGGTGGA GAAAACCAAT AAGATTAAAG |
| 1861 | ACAGGTATTG GGATTCTGGT CCCAGAGCTG ATCCCGTGGA AGATTTCCGG TACATCTGGG |
| 1921 | GCGGGTTTGC CTATCTGCAG GACATGGTTG AACAGGGGAT CACAAGGAGC CAGGTGCAGG |
| 1981 | CGGAGGCTCC AGTTGGAATC TACCTCCAGC AGATGCCCTA CCCCTGCTTC GTGGACGATT |
| 2041 | CTTTCATGAT CATCCTGAAC CGCTGTTTCC CTATCTTCAT GGTGCTGGCA TGGATCTACT |
| 2101 | CTGTCTCCAT GACTGTGAAG AGCATCGTCT TGGAGAAGGA GTTGCGACTG AAGGAGACCT |
| 2161 | TGAAAAATCA GGGTGTCTCC AATGCAGTGA TTTGGTGTAC CTGGTTCCTG GACAGCTTCT |
| 2221 | CCATCATGTC GATGAGCATC TTCCTCCTGA CGATATTCAT CATGCATGGA AGAATCCTAC |
| 2281 | ATTACAGCGA CCCATTCATC CTCTTCCTGT TCTTGTTGGC TTTCTCCACT GCCACCATCA |
| 2341 | TGCTGTGCTT TCTGCTCAGC ACCTTCTTCT CCAAGGCCAG TCTGGCAGCA GCCTGTAGTG |
| 2401 | GTGTCATCTA TTTCACCCTC TACCTGCCAC ACATCCTGTG CTTCGCCTGG CAGGACCGCA |
| 2461 | TGACCGCTGA GCTGAAGAAG GCTGTGAGCT TACTGTCTCC GGTGGCATTT GGATTTGGCA |
| 2521 | CTGAGTACCT GGTTCGCTTT GAAGAGCAAG GCCTGGGGCT GCAGTGGAGC AACATCGGGA |
| 2581 | ACAGTCCCAC GGAAGGGGAC GAATTCAGCT TCCTGCTGTC CATGCAGATG ATGCTCCTTG |
| 2641 | ATGCTGCTGT CTATGGCTTA CTCGCTTGGT ACCTTGATCA GGTGTTTCCA GGAGACTATG |
| 2701 | GAACCCCACT TCCTTGGTAC TTTCTTCTAC AAGAGTCGTA TTGGCTTGGC GGTGAAGGGT |
| 2761 | GTTCAACCAG AGAAGAAAGA GCCCTGGAAA AGACCGAGCC CCTAACAGAG GAAACGGAGG |
| 2821 | ATCCAGAGCA CCCAGAAGGA ATACACGACT CCTTCTTTGA ACGTGAGCAT CCAGGGTGGG |
| 2881 | TTCCTGGGGT ATGCGTGAAG AATCTGGTAA AGATTTTTGA GCCCTGTGGC CGGCCAGCTG |
| 2941 | TGGACCGTCT GAACATCACC TTCTACGAGA ACCAGATCAC CGCATTCCTG GGCCACAATG |
| 3001 | GAGCTGGGAA AACCACCACC TTGTCCATCC TGACGGGTCT GTTGCCACCA ACCTCTGGGA |
| 3061 | CTGTGCTCGT TGGGGGAAGG GACATTGAAA CCAGCCTGGA TGCAGTCCGG CAGAGCCTTG |
| 3121 | GCATGTGTCC ACAGCACAAC ATCCTGTTCC ACCACCTCAC GGTGGCTGAG CACATGCTGT |
| 3181 | TCTATGCCCA GCTGAAAGGA AAGTCCCAGG AGGAGGCCCA GCTGGAGATG GAAGCCATGT |
| 3241 | TGGAGGACAC AGGCCTCCAC CACAAGCGGA ATGAAGAGGC TCAGGACCTA TCAGGTGGCA |
| 3301 | TGCAGAGAAA GCTGTCGGTT GCCATTGCCT TTGTGGGAGA TGCCAAGGTG GTGATTCTGG |
| 3361 | ACGAACCCAC CTCTGGGGTG GACCCTTACT CGAGACGCTC AATCTGGGAT CTGCTCCTGA |
| 3421 | AGTATCGCTC AGGCAGAACC ATCATCATGT CCACTCACCA CATGGACGAG GCCGACCTCC |
| 3481 | TTGGGGACCG CATTGCCATC ATTGCCCAGG GAAGGCTCTA CTGCTCAGGC ACCCCACTCT |
| 3541 | TCCTGAAGAA CTGCTTTGGC ACAGGCTTGT ACTTAACCTT GGTGCGCAAG ATGAAAAACA |
| 3601 | TCCAGAGCCA AAGGAAAGGC AGTGAGGGGA CCTGCAGCTG CTCGTCTAAG GGTTTCTCCA |
| 3661 | CCACGTGTCC AGCCCACGTC GATGACCTAA CTCCAGAACA AGTCCTGGAT GGGGATGTAA |
| 3721 | ATGAGCTGAT GGATGTAGTT CTCCACCATG TTCCAGAGGC AAAGCTGGTG GAGTGCATTG |
| 3781 | GTCAAGAACT TATCTTCCTT CTTCCAAATA AGAACTTCAA GCACAGAGCA TATGCCAGCC |
| 3841 | TTTTCAGAGA GCTGGAGGAG ACGCTGGCTG ACCTTGGTCT CAGCAGTTTT GGAATTTCTG |
| 3901 | ACACTCCCCT GGAAGAGATT TTTCTGAAGG TCACGGAGGA TTCTGATTCA GGACCTCTGT |
| 3961 | TTGCGGGTGG CGCTCAGCAG AAAAGAGAAA ACGTCAACCC CCGACACCCC TGCTTGGGTC |
| 4021 | CCAGAGAGAA GGCTGGACAG ACACCCCAGG ACTCCAATGT CTGCTCCCCA GGGGCGCCGG |
| 4061 | CTGCTCACCC AGAGGGCCAG CCTCCCCCAG AGCCAGAGTG CCCAGGCCCG CAGCTCAACA |
| 4121 | CGGGGACACA GCTGGTCCTC CAGCATGTGC AGGCGCTGCT GGTCAAGAGA TTCCAACACA |
| 4181 | CCATCCGCAG CCACAAGGAC TTCCTGGCGC AGATCGTGCT CCCGGCTACC TTTGTGTTTT |
| 4241 | TGGCTCTGAT GCTTTCTATT GTTATCCCTC CTTTTGGCGA ATACCCCGCT TTGACCCTTC |
| 4301 | ACCCCTGGAT ATATGGGCAG CAGTACACCT TCTTCAGCAT GGATGAACCA GGCAGTGAGC |
| 4361 | AGTTCACGGT ACTTGCAGAC GTCCTCCTGA ATAAGCCAGG CTTTGGCAAC CGCTGCCTGA |
| 4421 | AGGAAGGGTG GCTTCCGGAG TACCCCTGTG GCAACTCAAC ACCCTGGAAG ACTCCTTCTG |
| 4481 | TGTCCCCAAA CATCACCCAG CTGTTCCAGA AGCAGAAATG GACACAGGTC AACCCTTCAC |
| 4541 | CATCCTGCAG GTGCAGCACC AGGGAGAAGC TCACCATGCT GCCAGAGTGC CCCGAGGGTG |
| 4601 | CCGGGGGCCT CCCGCCCCCC CAGAGAACAC AGCGCAGCAC GGAAATTCTA CAAGACCTGA |
| 4661 | CGGACAGGAA CATCTCCGAC TTCTTGGTAA AAACGTATCC TGCTCTTATA AGAAGCAGCT |
| 4721 | TAAAGAGCAA ATTCTGGGTC AATGAACAGA GGTATGGAGG AATTTCCATT GGAGGAAAGC |
| 4781 | TCCCAGTCGT CCCCATCACG GGGGAAGCAC TTGTTGGGTT TTTAAGCGAC CTTGGCCGGA |
| 4841 | TCATGAATGT GAGCGGGGGC CCTATCACTA GAGAGGCCTC TAAAGAAATA CCTGATTTCC |
| 4901 | TTAAACATCT AGAAACTGAA GACAACATTA AGGTGTGGTT TAATAACAAA GGCTGGCATG |
| 4961 | CCCTGGTCAG CTTTCTCAAT GTGGCCCACA ACGCCATCTT ACGGGCCAGC CTGCCTAAGG |
| 5021 | ACAGGAGCCC CGAGGAGTAT GGAATCACCG TCATTAGCCA ACCCCTGAAC CTGACCAAGG |
| 5081 | AGCAGCTCTC AGAGATTACA GTGCTGACCA CTTCAGTGGA TGCTGTGGTT GCCATCTGCG |
| 5141 | TGATTTTCTC CATGTCCTTC GTCCCAGCCA GCTTTGTCCT TTATTTGATC CAGGAGCGGG |
| 5201 | TGAACAAATC CAAGCACCTC CAGTTTATCA GTGGAGTGAG CCCCACCACC TACTGGGTGA |
| 5261 | CCAACTTCCT CTGGGACATC ATGAATTATT CCGTGAGTGC TGGGCTGGTG GTGGGCATCT |
| 5321 | TCATCGGGTT TCAGAAGAAA GCCTACACTT CTCCAGAAAA CCTTCCTGCC CTTGTGGCAC |
| 5381 | TGCTCCTGCT GTATGGATGG GCGGTCATTC CCATGATGTA CCCAGCATCC TTCCTGTTTG |
| 5441 | ATGTCCCCAG CACAGCCTAT GTGGCTTTAT CTTGTGCTAA TCTGTTCATC GGCATCAACA |
| 5501 | GCAGTGCTAT TACCTTCATC TTGGAATTAT TTGAGAATAA CCGGACGCTG CTCAGGTTCA |
| 5561 | ACGCCGTGCT GAGGAAGCTG CTCATTGTCT TCCCCCACTT CTGCCTGGGC CGGGGCCTCA |
| 5621 | TTGACCTTGC ACTGAGCCAG GCTGTGACAG ATGTCTATGC CCGGTTTGGT GAGGAGCACT |
| 5681 | CTGCAAATCC GTTCCACTGG GACCTGATTG GGAAGAACCT GTTTGCCATG GTGGTGGAAG |
| 5741 | GGGTGGTGTA CTTCCTCCTG ACCCTGCTGG TCCAGCGCCA CTTCTTCCTC TCCCAATGGA |
| 5801 | TTGCCGAGCC CACTAAGGAG CCCATTGTTG ATGAAGATGA TGATGTGGCT GAAGAAAGAC |
| 5861 | AAAGAATTAT TACTGGTGGA AATAAAACTG ACATCTTAAG GCTACATGAA CTAACCAAGA |
| 5921 | TTTATCCAGG CACCTCCAGC CCAGCAGTGG ACAGGCTGTG TGTCGGAGTT CGCCCTGGAG |
| 5981 | AGTGCTTTGG CCTCCTGGGA GTGAATGGTG CCGGCAAAAC AACCACATTC AAGATGCTCA |
| 6041 | CTGGGGACAC CACAGTGACC TCAGGGGATG CCACCGTAGC AGGCAAGAGT ATTTTAACCA |
| 6101 | ATATTTCTGA AGTCCATCAA AATATGGGCT ACTGTCCTCA GTTTGATGCA ATTGATGAGC |
| 6161 | TGCTCACAGG ACGAGAACAT CTTTACCTTT ATGCCCGGCT TCGAGGTGTA CCAGCAGAAG |
| 6221 | AAATCGAAAA GGTTGCAAAC TGGAGTATTA AGAGCCTGGG CCTGACTGTC TACGCCGACT |
| 6281 | GCCTGGCTGG CACGTACAGT GGGGGCAACA AGCGGAAACT CTCCACAGCC ATCGCACTCA |
| 6341 | TTGGCTGCCC ACCGCTGGTG CTGCTGGATG AGCCCACCAC AGGGATGGAC CCCCAGGCAC |
| 6401 | GCCGCATGCT GTGGAACGTC ATCGTGAGCA TCATCAGAGA AGGGAGGGCT GTGGTCCTCA |
| 6461 | CATCCCACAG CATGGAAGAA TGTGAGGCAC TGTGTACCCG GCTGGCCATC ATGGTAAAGG |
| 6521 | GCGCCTTTCG ATGTATGGGC ACCATTCAGC ATCTCAAGTC CAAATTTGGA GATGGCTATA |
| 6581 | TCGTCACAAT GAAGATCAAA TCCCCGAAGG ACGACCTGCT TCCTGACCTG AACCCTGTGG |
| 6641 | AGCAGTTCTT CCAGGGGAAC TTCCCAGGCA GTGTGCAGAG GGAGAGGCAC TACAACATGC |
| 6701 | TCCAGTTCCA GGTCTCCTCC TCCTCCCTGG CGAGGATCTT CCAGCTCCTC CTCTCCCACA |
| 6761 | AGGACAGCCT GCTCATCGAG GAGTACTCAG TCACACAGAC CACACTGGAC CAGGTGTTTG |
| 6821 | TAAATTTTGC TAAACAGCAG ACTGAAAGTC ATGACCTCCC TCTGCACCCT CGAGCTGCTG |
| 6881 | GAGCCAGTCG ACAAGCCCAG GACTGATCTT TCACACCGCT CGTTCCTGCA GCCAGAAAGG |
| 6941 | AACTCTGGGC AGCTGGAGGC GCAGGAGCCT GTGCCCATAT GGTCATCCAA ATGGACTGGC |
| 7001 | CAGCGTAAAT GACCCCACTG CAGCAGAAAA CAAACACACG AGGAGCATGC AGCGAATTCA |
| 7061 | GAAAGAGGTC TTTCAGAAGG AAACCGAAAC TGACTTGCTC ACCTGGAACA CCTGATGGTG |
| 7121 | AAACCAAACA AATACAAAAT CCTTCTCCAG ACCCCAGAAC TAGAAACCCC GGGCCATCCC |
| 7181 | ACTAGCAGCT TTGGCCTCCA TATTGCTCTC ATTTCAAGCA GATCTGCTTT TCTGCATGTT |
| 7241 | TGTCTGTGTG TCTGCGTTGT GTGTGATTTT CATGGAAAAA TAAAATGCAA ATGCACTCAT |
| 7301 | CACAAA. |

In some embodiments, the sequence encoding ABCA4 comprises a modified nucleotide sequence. In some embodiments, the sequence encoding ABCA4 comprises a nucleotide sequence that has at least 70%, at least 75%, at least 80%, at least 85%, at least 90%, at least 95%, at least 97%, at least 99% or any percentage in between of identity to the nucleotide sequence of:

| (SEQ ID NO: 2) | |
|---|---|
| 1 | AGGACACAGC GTCCGGAGCC AGAGGCGCTC TTAACGGCGT TTATGTCCTT TGCTGTCTGA |
| 61 | GGGGCCTCAG CTCTGACCAA TCTGGTCTTC GTGTGGTCAT TAGCATGGGC TTCGTGAGAC |
| 121 | AGATACAGCT TTTGCTCTGG AAGAACTGGA CCCTGCGGAA AAGGCAAAAG ATTCGCTTTG |
| 181 | TGGTGGAACT CGTGTGGCCT TTATCTTTAT TTCTGGTCTT GATCTGGTTA AGGAATGCCA |
| 241 | ACCCGCTCTA CAGCCATCAT GAATGCCATT TCCCCAACAA GGCGATGCCC TCAGCAGGAA |
| 301 | TGCTGCCGTG GCTCCAGGGG ATCTTCTGCA ATGTGAACAA TCCCTGTTTT CAAAGCCCCA |
| 361 | CCCCAGGAGA ATCTCCTGGA ATTGTGTCAA ACTATAACAA CTCCATCTTG GCAAGGGTAT |
| 421 | ATCGAGATTT TCAAGAACTC CTCATGAATG CACCAGAGAG CCAGCACCTT GGCCGTATTT |
| 481 | GGACAGAGCT ACACATCTTG TCCCAATTCA TGGACACCCT CCGGACTCAC CCGGAGAGAA |
| 541 | TTGCAGGAAG AGGAATACGA ATAAGGGATA TCTTGAAAGA TGAAGAAACA CTGACACTAT |
| 601 | TTCTCATTAA AAACATCGGC CTGTCTGACT CAGTGGTCTA CCTTCTGATC AACTCTCAAG |
| 661 | TCCGTCCAGA GCAGTTCGCT CATGGAGTCC CGGACCTGGC GCTGAAGGAC ATCGCCTGCA |
| 721 | GCGAGGCCCT CCTGGAGCGC TTCATCATCT TCAGCCAGAG ACGCGGGGCA AAGACGGTGC |
| 781 | GCTATGCCCT GTGCTCCCTC TCCCAGGGCA CCCTACAGTG GATAGAAGAC ACTCTGTATG |
| 841 | CCAACGTGGA CTTCTTCAAG CTCTTCCGTG TGCTTCCCAC ACTCCTAGAC AGCCGTTCTC |
| 901 | AAGGTATCAA TCTGAGATCT TGGGGAGGAA TATTATCTGA TATGTCACCA AGAATTCAAG |
| 961 | AGTTTATCCA TCGGCCGAGT ATGCAGGACT TGCTGTGGGT GACCAGGCCC CTCATGCAGA |
| 1021 | ATGGTGGTCC AGAGACCTTT ACAAAGCTGA TGGGCATCCT GTCTGACCTC CTGTGTGGCT |
| 1081 | ACCCCGAGGG AGGTGGCTCT CGGGTGCTCT CCTTCAACTG GTATGAAGAC AATAACTATA |
| 1141 | AGGCCTTTCT GGGGATTGAC TCCACAAGGA AGGATCCTAT CTATTCTTAT GACAGAAGAA |
| 1201 | CAACATCCTT TTGTAATGCA TTGATCCAGA GCCTGGAGTC AAATCCTTTA ACCAAAATCG |
| 1261 | CTTGGAGGGC GGCAAAGCCT TTGCTGATGG GAAAAATCCT GTACACTCCT GATTCACCTG |
| 1321 | CAGCACGAAG GATACTGAAG AATGCCAACT CAACTTTTGA AGAACTGGAA CACGTTAGGA |
| 1381 | AGTTGGTCAA AGCCTGGGAA GAAGTAGGGC CCCAGATCTG GTACTTCTTT GACAACAGCA |
| 1441 | CACAGATGAA CATGATCAGA GATACCCTGG GGAACCCAAC AGTAAAAGAC TTTTTGAATA |
| 1501 | GGCAGCTTGG TGAAGAAGGT ATTACTGCTG AAGCCATCCT AAACTTCCTC TACAAGGGCC |
| 1561 | CTCGGGAAAG CCAGGCTGAC GACATGGCCA ACTTCGACTG GAGGGACATA TTTAACATCA |
| 1621 | CTGATCGCAC CCTCCGCCTT GTCAATCAAT ACCTGGAGTG CTTGGTCCTG GATAAGTTTG |
| 1681 | AAAGCTACAA TGATGAAACT CAGCTCACCC AACGTGCCCT CTCTCTACTG GAGGAAAACA |
| 1741 | TGTTCTGGGC CGGAGTGGTA TTCCCTGACA TGTATCCCTG GACCAGCTCT CTACCACCCC |
| 1801 | ACGTGAAGTA TAAGATCCGA ATGGACATAG ACGTGGTGGA GAAAACCAAT AAGATTAAAG |
| 1861 | ACAGGTATTG GGATTCTGGT CCCAGAGCTG ATCCCGTGGA AGATTTCCGG TACATCTGGG |
| 1921 | GCGGGTTTGC CTATCTGCAG GACATGGTTG AACAGGGGAT CACAAGGAGC CAGGTGCAGG |
| 1981 | CGGAGGCTCC AGTTGGAATC TACCTCCAGC AGATGCCCTA CCCCTGCTTC GTGGACGATT |
| 2041 | CTTTCATGAT CATCCTGAAC CGCTGTTTCC CTATCTTCAT GGTGCTGGCA TGGATCTACT |
| 2101 | CTGTCTCCAT GACTGTGAAG AGCATCGTCT TGGAGAAGGA GTTGCGACTG AAGGAGACCT |
| 2161 | TGAAAAATCA GGGTGTCTCC AATGCAGTGA TTTGGTGTAC CTGGTTCCTG GACAGCTTCT |
| 2221 | CCATCATGTC GATGAGCATC TTCCTCCTGA CGATATTCAT CATGCATGGA AGAATCCTAC |
| 2281 | ATTACAGCGA CCCATTCATC CTCTTCCTGT TCTTGTTGGC TTTCTCCACT GCCACCATCA |
| 2341 | TGCTGTGCTT TCTGCTCAGC ACCTTCTTCT CCAAGGCCAG TCTGGCAGCA GCCTGTAGTG |
| 2401 | GTGTCATCTA TTTCACCCTC TACCTGCCAC ACATCCTGTG CTTCGCCTGG CAGGACCGCA |
| 2461 | TGACCGCTGA GCTGAAGAAG GCTGTGAGCT TACTGTCTCC GGTGGCATTT GGATTTGGCA |
| 2521 | CTGAGTACCT GGTTCGCTTT GAAGAGCAAG GCCTGGGGCT GCAGTGGAGC AACATCGGGA |
| 2581 | ACAGTCCCAC GGAAGGGGAC GAATTCAGCT TCCTGCTGTC CATGCAGATG ATGCTCCTTG |
| 2641 | ATGCTGCTGT CTATGGCTTA CTCGCTTGGT ACCTTGATCA GGTGTTTCCA GGAGACTATG |
| 2701 | GAACCCCACT TCCTTGGTAC TTTCTTCTAC AAGAGTCGTA TTGGCTTGGC GGTGAAGGGT |
| 2761 | GTTCAACCAG AGAAGAAAGA GCCCTGGAAA AGACCGAGCC CCTAACAGAG GAAACGGAGG |
| 2821 | ATCCAGAGCA CCCAGAAGGA ATACACGACT CCTTCTTTGA ACGTGAGCAT CCAGGGTGGG |
| 2881 | TTCCTGGGGT ATGCGTGAAG AATCTGGTAA AGATTTTTGA GCCCTGTGGC CGGCCAGCTG |
| 2941 | TGGACCGTCT GAACATCACC TTCTACGAGA ACCAGATCAC CGCATTCCTG GGCCACAATG |
| 3001 | GAGCTGGGAA AACCACCACC TTGTCCATCC TGACGGGTCT GTTGCCACCA ACCTCTGGGA |
| 3061 | CTGTGCTCGT TGGGGGAAGG GACATTGAAA CCAGCCTGGA TGCAGTCCGG CAGAGCCTTG |
| 3121 | GCATGTGTCC ACAGCACAAC ATCCTGTTCC ACCACCTCAC GGTGGCTGAG CACATGCTGT |
| 3181 | TCTATGCCCA GCTGAAAGGA AAGTCCCAGG AGGAGGCCCA GCTGGAGATG GAAGCCATGT |
| 3241 | TGGAGGACAC AGGCCTCCAC CACAAGCGGA ATGAAGAGGC TCAGGACCTA TCAGGTGGCA |
| 3301 | TGCAGAGAAA GCTGTCGGTT GCCATTGCCT TTGTGGGAGA TGCCAAGGTG GTGATTCTGG |
| 3361 | ACGAACCCAC CTCTGGGGTG GACCCTTACT CGAGACGCTC AATCTGGGAT CTGCTCCTGA |
| 3421 | AGTATCGCTC AGGCAGAACC ATCATCATGT CCACTCACCA CATGGACGAG GCCGACCTCC |
| 3481 | TTGGGGACCG CATTGCCATC ATTGCCCAGG GAAGGCTCTA CTGCTCAGGC ACCCCACTCT |
| 3541 | TCCTGAAGAA CTGCTTTGGC ACAGGCTTGT ACTTAACCTT GGTGCGCAAG ATGAAAAACA |
| 3601 | TCCAGAGCCA AAGGAAAGGC AGTGAGGGGA CCTGCAGCTG CTCGTCTAAG GGTTTCTCCA |
| 3661 | CCACGTGTCC AGCCCACGTC GATGACCTAA CTCCAGAACA AGTCCTGGAT GGGGATGTAA |
| 3721 | ATGAGCTGAT GGATGTAGTT CTCCACCATG TTCCAGAGGC AAAGCTGGTG GAGTGCATTG |
| 3781 | GTCAAGAACT TATCTTCCTT CTTCCAAATA AGAACTTCAA GCACAGAGCA TATGCCAGCC |
| 3841 | TTTTCAGAGA GCTGGAGGAG ACGCTGGCTG ACCTTGGTCT CAGCAGTTTT GGAATTTCTG |
| 3901 | ACACTCCCCT GGAAGAGATT TTTCTGAAGG TCACGGAGGA TTCTGATTCA GGACCTCTGT |
| 3961 | TTGCGGGTGG CGCTCAGCAG AAAAGAGAAA ACGTCAACCC CCGACACCCC TGCTTGGGTC |
| 4021 | CCAGAGAGAA GGCTGGACAG ACACCCCAGG ACTCCAATGT CTGCTCCCCA GGGGCGCCGG |
| 4081 | CTGCTCACCC AGAGGGCCAG CCTCCCCCAG AGCCAGAGTG CCCAGGCCCG CAGCTCAACA |
| 4141 | CGGGGACACA GCTGGTCCTC CAGCATGTGC AGGCGCTGCT GGTCAAGAGA TTCCAACACA |
| 4201 | CCATCCGCAG CCACAAGGAC TTCCTGGCGC AGATCGTGCT CCCGGCTACC TTTGTGTTTT |
| 4261 | TGGCTCTGAT GCTTTCTATT GTTATCCCTC CTTTTGGCGA ATACCCCGCT TTGACCCTTC |
| 4321 | ACCCCTGGAT ATATGGGCAG CAGTACACCT TCTTCAGCAT GGATGAACCA GGCAGTGAGC |
| 4381 | AGTTCACGGT ACTTGCAGAC GTCCTCCTGA ATAAGCCAGG CTTTGGCAAC CGCTGCCTGA |
| 4441 | AGGAAGGGTG GCTTCCGGAG TACCCCTGTG GCAACTCAAC ACCCTGGAAG ACTCCTTCTG |
| 4501 | TGTCCCCAAA CATCACCCAG CTGTTCCAGA AGCAGAAATG GACACAGGTC AACCCTTCAC |
| 4561 | CATCCTGCAG GTGCAGCACC AGGGAGAAGC TCACCATGCT GCCAGAGTGC CCCGAGGGTG |
| 4621 | CCGGGGGCCT CCCGCCCCCC CAGAGAACAC AGCGCAGCAC GGAAATTCTA CAAGACCTGA |
| 4681 | CGGACAGGAA CATCTCCGAC TTCTTGGTAA AAACGTATCC TGCTCTTATA AGAAGCAGCT |
| 4741 | TAAAGAGCAA ATTCTGGGTC AATGAACAGA GGTATGGAGG AATTTCCATT GGAGGAAAGC |
| 4801 | TCCCAGTCGT CCCCATCACG GGGGAAGCAC TTGTTGGGTT TTTAAGCGAC CTTGGCCGGA |
| 4861 | TCATGAATGT GAGCGGGGGC CCTATCACTA GAGAGGCCTC TAAAGAAATA CCTGATTTCC |
| 4921 | TTAAACATCT AGAAACTGAA GACAACATTA AGGTGTGGTT TAATAACAAA GGCTGGCATG |
| 4981 | CCCTGGTCAG CTTTCTCAAT GTGGCCCACA ACGCCATCTT ACGGGCCAGC CTGCCTAAGG |
| 5041 | ACAGGAGCCC CGAGGAGTAT GGAATCACCG TCATTAGCCA ACCCCTGAAC CTGACCAAGG |
| 5101 | AGCAGCTCTC AGAGATTACA GTGCTGACCA CTTCAGTGGA TGCTGTGGTT GCCATCTGCG |
| 5161 | TGATTTTCTC CATGTCCTTC GTCCCAGCCA GCTTTGTCCT TTATTTGATC CAGGAGCGGG |
| 5221 | TGAACAAATC CAAGCACCTC CAGTTTATCA GTGGAGTGAG CCCCACCACC TACTGGGTAA |
| 5281 | CCAACTTCCT CTGGGACATC ATGAATTATT CCGTGAGTGC TGGGCTGGTG GTGGGCATCT |
| 5341 | TCATCGGGTT TCAGAAGAAA GCCTACACTT CTCCAGAAAA CCTTCCTGCC CTTGTGGCAC |
| 5401 | TGCTCCTGCT GTATGGATGG GCGGTCATTC CCATGATGTA CCCAGCATCC TTCCTGTTTG |
| 5461 | ATGTCCCCAG CACAGCCTAT GTGGCTTTAT CTTGTGCTAA TCTGTTCATC GGCATCAACA |
| 5521 | GCAGTGCTAT TACCTTCATC TTGGAATTAT TTGAGAATAA CCGGACGCTG CTCAGGTTCA |
| 5581 | ACGCCGTGCT GAGGAAGCTG CTCATTGTCT TCCCCCACTT CTGCCTGGGC CGGGGCCTCA |
| 5641 | TTGACCTTGC ACTGAGCCAG GCTGTGACAG ATGTCTATGC CCGGTTTGGT GAGGAGCACT |
| 5701 | CTGCAAATCC GTTCCACTGG GACCTGATTG GGAAGAACCT GTTTGCCATG GTGGTGGAAG |
| 5761 | GGGTGGTGTA CTTCCTCCTG ACCCTGCTGG TCCAGCGCCA CTTCTTCCTC TCCCAATGGA |
| 5821 | TTGCCGAGCC CACTAAGGAG CCCATTGTTG ATGAAGATGA TGATGTGGCT GAAGAAAGAC |
| 5881 | AAAGAATTAT TACTGGTGGA AATAAAACTG ACATCTTAAG GCTACATGAA CTAACCAAGA |
| 5941 | TTTATCCAGG CACCTCCAGC CCAGCAGTGG ACAGGCTGTG TGTCGGAGTT CGCCCTGGAG |
| 6001 | AGTGCTTTGG CCTCCTGGGA GTGAATGGTG CCGGCAAAAC AACCACATTC AAGATGCTCA |
| 6061 | CTGGGGACAC CACAGTGACC TCAGGGGATG CCACCGTAGC AGGCAAGAGT ATTTTAACCA |
| 6121 | ATATTTCTGA AGTCCATCAA AATATGGGCT ACTGTCCTCA GTTTGATGCA ATCGATGAGC |
| 6181 | TGCTCACAGG ACGAGAACAT CTTTACCTTT ATGCCCGGCT TCGAGGTGTA CCAGCAGAAG |
| 6241 | AAATCGAAAA GGTTGCAAAC TGGAGTATTA AGAGCCTGGG CCTGACTGTC TACGCCGACT |
| 6301 | GCCTGGCTGG CACGTACAGT GGGGGCAACA AGCGGAAACT CTCCACAGCC ATCGCACTCA |
| 6361 | TTGGCTGCCC ACCGCTGGTG CTGCTGGATG AGCCCACCAC AGGGATGGAC CCCCAGGCAC |
| 6421 | GCCGCATGCT GTGGAACGTC ATCGTGAGCA TCATCAGAGA AGGGAGGGCT GTGGTCCTCA |
| 6481 | CATCCCACAG CATGGAAGAA TGTGAGGCAC TGTGTACCCG GCTGGCCATC ATGGTAAAGG |
| 6541 | GCGCCTTTCG ATGTATGGGC ACCATTCAGC ATCTCAAGTC CAAATTTGGA GATGGCTATA |
| 6601 | TCGTCACAAT GAAGATCAAA TCCCCGAAGG ACGACCTGCT TCCTGACCTG AACCCTGTGG |
| 6661 | AGCAGTTCTT CCAGGGGAAC TTCCCAGGCA GTGTGCAGAG GGAGAGGCAC TACAACATGC |
| 6721 | TCCAGTTCCA GGTCTCCTCC TCCTCCCTGG CGAGGATCTT CCAGCTCCTC CTCTCCCACA |
| 6781 | AGGACAGCCT GCTCATCGAG GAGTACTCAG TCACACAGAC CACACTGGAC CAGGTGTTTG |
| 6841 | TAAATTTTGC TAAACAGCAG ACTGAAAGTC ATGACCTCCC TCTGCACCCT CGAGCTGCTG |
| 6901 | GAGCCAGTCG ACAAGCCCAG GACTGATCTT TCACACCGCT CGTTCCTGCA GCCAGAAAGG |
| 6961 | AACTCTGGGC AGCTGGAGGC GCAGGAGCCT GTGCCCATAT GGTCATCCAA ATGGACTGGC |
| 7021 | CAGCGTAAAT GACCCCACTG CAGCAGAAAA CAAACACACG AGGAGCATGC AGCGAATTCA |
| 7081 | GAAAGAGGTC TTTCAGAAGG AAACCGAAAC TGACTTGCTC ACCTGGAACA CCTGATGGTG |
| 7141 | AAACCAAACA AATACAAAAT CCTTCTCCAG ACCCCAGAAC TAGAAACCCC GGGCCATCCC |
| 7201 | ACTAGCAGCT TTGGCCTCCA TATTGCTCTC ATTTCAAGCA GATCTGCTTT TCTGCATGTT |
| 7261 | TGTCTGTGTG TCTGCGTTGT GTGTGATTTT CATGGAAAAA TAAAATGCAA ATGCACTCAT |
| 7321 | CACAAA. |

In some embodiments of the compositions of the disclosure, the ABCA4 construct comprises a promoter. In some embodiments, the promoter comprises a rhodopsin kinase promoter. In some embodiments, the rhodopsin kinase promoter is isolated or derived from the promoter of the G protein-coupled receptor kinase 1 (GRK1) gene. In some embodiments, the promoter is a GRK1 promoter. In some embodiments, the sequence encoding the GRK1 promoter comprises a sequence having at least 80% identity, at least 90% identity, at least 95% identity, at least 97% identity or at least 99% identity to:

| (SEQ ID NO: 75) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments, the GRK1 promoter comprises or consists of:

| (SEQ ID NO: 75) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments of the compositions of the disclosure, the ABCA4 construct comprises a promoter. In some embodiments, the promoter comprises a chicken beta-actin (CBA) promoter. In some embodiments, the sequence encoding the CBA promoter comprises a sequence having at least 80% identity, at least 90% identity, at least 95% identity, at least 97% identity or at least 99% identity to:

| (SEQ ID NO: 16) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |
| or | |
| (SEQ ID NO: 24) | |
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

In some embodiments, the CBA promoter comprises or consists of:

| (SEQ ID NO: 16) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |
| or | |
| (SEQ ID NO: 24) | |
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

In some embodiments of the compositions of the disclosure, the ABCA4 construct comprises a promoter variant, e.g., a CMV.CBA promoter, a CBA.RBG promoter, or a CBA.InEx promoter.

In some embodiments, the promoter comprises a CMV.CBA promoter variant, e.g., comprising CMV enhancer and a CBA promoter. In some embodiments, the sequence encoding the CMV.CBA promoter comprises a sequence having at least 80% identity, at least 90% identity, at least 95% identity, at least 97% identity or at least 99% identity to:

(SEQ ID NO: 84)
CTCAGATCTGAATTCGGTACCTAGTTATTAATAGTAATCAATTACGGGGTC
ATTAGTTCATAGCCCATATATGGAGTTCCGCGTTACATAACTTACGGTAAA
TGGCCCGCCTGGCTGACCGCCCAACGACCCCCGCCCATTGACGTCAATAAT
GACGTATGTTCCCATAGTAACGCCAATAGGGACTTTCCATTGACGTCAATG
GGTGGAGTATTTACGGTAAACTGCCCACTTGGCAGTACATCAAGTGTATCA
TATGCCAAGTACGCCCCCTATTGACGTCAATGACGGTAAATGGCCCGCCTG
GCATTATGCCCAGTACATGACCTTATGGGACTTTCCTACTTGGCAGTACAT
CTACGTATTAGTCATCGCTATTACCATGGTCGAGGTGAGCCCCACGTTCTG
CTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGCG
CGCGCCAGGCGGGGCGGGGCGGGGCGAGGGGCGGGGCGGGGCGAGGCGGAG
AGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGC
G.

In some embodiments, the promoter comprises a CBA.RBG promoter variant, e.g., comprising a CBA promoter and a RGB intron. In some embodiments, the sequence encoding the CBA.RBG promoter comprises a sequence having at least 80% identity, at least 90% identity, at least 95% identity, at least 97% identity or at least 99% identity to:

(SEQ ID NO: 85)
TCGAGGTGAG CCCCACGTTC TGCTTCACTC TCCCCATCTC
CCCCCCCTCC CCACCCCCAA TTTTGTATTT ATTTATTTTT
TAATTATTTT GTGCAGCGAT GGGGGCGGGG GGGGGGGGGG
GGCGCGCGCC AGGCGGGGCG GGGCGGGGCG AGGGGCGGGG
CGGGGCGAGG CGGAGAGGTG CGGCGGCAGC CAATCAGAGC
GGCGCGCTCC GAAAGTTTCC TTTTATGGCG AGGCGGCGGC
GGCGGCGGCC CTATAAAAAG CGAAGCGCGC GGCGGGCGGG
AGTCGCTGCG CGCTGCCTTC GCCCCGTGCC CCGCTCCGCC
GCCGCCTCGC GCCGCCCGCC CCGGCTCTGA CTGACCGCGT
TACTCCCACA GGTGAGCGGG CGGGACGGCC CTTCTCCTCC
GGGCTGTAAT TAGCGCTTGG TTTAATGACG GCTTGTTTCT
TTTCTGTGGC TGCGTGAAAG CCTTGAGGGG CTCCGGGAGG
GCCCTTTGTG CGGGGGGAGC GGCTCGGGGC TGTCCGCGGG
GGGACGGCTG CCTTCGGGGG GGACGGGGCA GGGCGGGGTT
CGGCTTCTGG CGTGTGACCG GCGGCTCTAG AGCCTCTGCT
AACCATGTTC ATGCCTTCTT CTTTTTCCTA CAGCTCCTGG
GCAACGTGCT GGTTATTGTG CTGTCTCATC ATTTTGGCAA
AGAATT

In some embodiments, the promoter comprises a CBA.InEx promoter variant, e.g., comprising a CBA promoter, an intron, and an exon. In some embodiments, the sequence encoding the CBA.InEx promoter comprises a sequence having at least 80% identity, at least 90% identity, at least 95% identity, at least 97% identity or at least 99% identity to (the intron is italicized):

(SEQ ID NO: 86)
TCGAGGTGAG CCCCACGTTC TGCTTCACTC TCCCCATCTC
CCCCCCCTCC CCACCCCCAA TTTTGTATTT ATTTATTTTT
TAATTATTTT GTGCAGCGAT GGGGGCGGGG GGGGGGGGGG
GGCGCGCGCC AGGCGGGGCG GGGCGGGGCG AGGGGCGGGG
CGGGGCGAGG CGGAGAGGTG CGGCGGCAGC CAATCAGAGC
GGCGCGCTCC GAAAGTTTCC TTTTATGGCG AGGCGGCGGC
GGCGGCGGCC CTATAAAAAG CGAAGCGCGC GGCGGGCGTG
CCGCAGGGGG ACGGCTGCCT TCGGGGGGGA CGGGGCAGGG
CGGGGTTCGG CTTCTGGCGT GTGACCGGCG GCTCTAGAGC
CTCTGCTAAC CATGTTCATG CCTTCTTCTT TTTCCTACAG
CTCCTGGGCA ACGTGCTGGT TATTGTGCTG TCTCATCATT

In some embodiments of the compositions of the disclosure, the ABCA4 construct comprises a polyadenylation signal. In some embodiments, the sequence encoding the polyA signal comprises a polyA signal isolated or derived from a bovine growth hormone (BGH) polyA signal. In some embodiments, the BGH polyA signal comprises a nucleotide sequence that has at least 80% identity, at least 97% identity or 100% identity to the nucleotide sequence of:

| (SEQ ID NO: 83) | |
|---|---|
| 1 | cgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg. |

In some embodiments, the sequence encoding the BGH polyA comprises or consists of the nucleotide sequence of:

| (SEQ ID NO: 83) | |
|---|---|
| 1 | cgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg. |

In some embodiments of the compositions of the disclosure, the ABCA4 construct further comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2). In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type AAV2 sequence. In some embodiments, the variation comprises a substitution, an insertion, a deletion, an inversion, or a transposition. In some embodiments, the variation comprises a truncation or an elongation of a wild type or a variant sequence. In some embodiments, the ITRs are derived from a 3′ AAV2 ITR in forward and reverse orientation with subsequent deletions, to produce stabilized ITRs. In certain embodiments, the 5′ ITR comprises or consists of the following sequence:

(SEQ ID NO: 36)
CTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGA
GTGGCCAACTCCATCACTAGGGGTTCCT.

In some embodiments, the 3′ ITR comprises or consists of the following sequence:

(SEQ ID NO: 37)
AGGAACCCCTAGTGATGGAGTTGGCCACTCCCTCTCTGCGCGCTCGCTCGC
TCACTGAGGCCGGGCGACCAAAGGTCGCCCGACGCCCGGGCGGCCTCAGTG
AGCGAGCGAGCGCGCAGAG.

In some embodiments, the sequence encoding the 5′ ITR comprises the sequence of

(SEQ ID NO: 34)
CTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTC
CATCACTAGGGGTTCCT.

In some embodiments, the sequence encoding a 3′ ITR comprises a wild type sequence isolated or derived of an AAV2. In some embodiments, the sequence encoding the 3′ ITR comprises the sequence of

(SEQ ID NO: 35)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

In some embodiments of the compositions of the disclosure, an AAV comprises a viral sequence essential for formation of a replication-deficient AAV. In some embodiments, the viral sequence is isolated or derived from an AAV of the same serotype as one or both of the sequence encoding the 5′ITR or the sequence encoding the 3′ITR. In some embodiments, the viral sequence, the sequence encoding the 5′ITR or the sequence encoding the 3′ITR are isolated or derived from an AAV2.

In some embodiments of the compositions of the disclosure, an AAV comprises a viral sequence essential for formation of a replication-deficient AAV, a sequence encoding the 5′ITR and a sequence encoding the 3′ITR, but does not comprise any other sequence isolated or derived from an AAV. In some embodiments, the AAV is a recombinant AAV (rAAV), comprising a viral sequence essential for formation of a replication-deficient AAV, a sequence encoding the 5′ITR, a sequence encoding the 3′ITR, and a sequence encoding an ABCA4 construct of the disclosure.

In some embodiments, a plasmid DNA used to create the rAAV in a host cell comprises a selection marker. Exemplary selection markers include, but are not limited to, antibiotic resistance genes. Exemplary antibiotic resistance genes include, but are not limited to, ampicillin and kanamycin. Exemplary selection markers include, but are not limited to, drug or small molecule resistance genes. Exemplary selection markers include, but are not limited to, dapD and a repressible operator including but not limited to a lacO/P construct controlling or suppressing dapD expression, wherein plasmid selection is performed by administering or contacting a transformed cell with a plasmid capable of operator repressor titration (ORT). Exemplary selection markers include, but are not limited to, a ccd selection gene. In some embodiments, the ccd selection gene comprises a sequence encoding a ccdA selection gene that rescues a host cell line engineered to express a toxic ccdB gene. Exemplary selection markers include, but are not limited to, sacB, wherein an RNA is administered or contacted to a host cell to suppress expression of the sacB gene in sucrose media. Exemplary selection markers include, but are not limited to, a segregational killing mechanism such as the parAB+ locus composed of Hok (a host killing gene) and Sok (suppression of killing).

AAV-ABCA4 Dual Vector Constructs

AAV is a small virus that presents very low immunogenicity and is not associated with any known human disease. The lack of an associated inflammatory response means that AAV does not cause retinal damage when injected into the eye.

However, the size of the AAV capsid imposes a limit on the amount of DNA that can be packaged within it. The AAV genome is approximately 4.7 kilobases (kb) in size, and it is believed that the corresponding upper size limit for DNA packaging in AAV is approximately 5 kb. The coding sequence of the ABCA4 gene is approximately 6.8 kb in size (with further genetic elements being required for gene expression), making it too large to be incorporated into a standard AAV vector.

“Dual” Vectors

An alternative approach has been to prepare dual vector systems, in which a transgene larger than the approximately 5 kb limit is split approximately in half into two separate vectors of defined sequence: an “upstream” vector containing the 5′ portion of the transgene, and a “downstream” vector containing the 3′ portion of the transgene. Transduction of a target cell by both upstream and downstream vectors allows a full-length transgene to be re-assembled from the two fragments using a variety of intracellular mechanisms. Methods disclosed herein may be used to produce either or both vector of a dual vector system. Compositions disclosed herein may comprise one or both vectors of a dual vector system.

Dual vector systems of the disclosure use an “overlapping” approach. In an overlapping dual vector system, part of the coding sequence at the 3′ end of the upstream coding sequence portion overlaps with a homologous sequence at the 5′ of the downstream coding sequence portion. Upon transduction of a target cell by upstream and downstream vectors, homologous recombination between the upstream and downstream portions of coding sequence allows for the recreation of a full-length transgene, from which a corresponding mRNA can be transcribed and full-length protein expressed.

Without wishing to be bound by any particular theory, a full length transgene (e.g. ABCA4) may be generated from an overlapping dual vector system by second strand synthesis, followed by homologous recombination. Upon transduction of cell by an upstream AAV particle and a downstream particle, a corresponding ssDNA upstream AAV vector and a downstream AAV vector is released into the cell or a nucleus thereof, and a dsDNA comprising the 5′ (upstream) portion of the transgene and the 3′ (downstream) portion of the transgene are generated from each of the ssDNAs by second strand synthesis. The dsDNA then undergoes homologous recombination at the region of overlap between the upstream and downstream portions of coding sequence, which allows for the recreation of a full-length transgene, from which a corresponding mRNA can be transcribed and full-length protein expressed. For example, WO 2014/170480 describes a dual AAV vector system encoding a human ABCA4 protein (the contents of which are incorporated herein in their entirety).

In some embodiments of the compositions and methods of the disclosure, a first AAV vector comprises a 5′ portion of an ABCA4 coding sequence. In some embodiments, a second AAV vector comprises a 3′ portion of an ABCA4 coding sequence. In some embodiments, the 5′ end portion and the 3′ end portion overlap by at least about 20 nucleotides. In some embodiments, the first AAV vector and the second AAV vector each comprise a single stranded DNA (ssDNA). In some embodiments, the first AAV vector comprises a sequence of the ABCA4 coding sequences and/or a sequence complementary to the ABCA4 coding sequence. In some embodiments, the second AAV vector comprises a sequence of the ABCA4 coding sequences and/or a sequence complementary to the ABCA4 coding sequence. In some embodiments, the first AAV vector comprises a sequence of the 5′ ABCA4 coding sequences and a sequence complementary to a portion of the 3′ ABCA4 coding sequence. In some embodiments, the second AAV vector comprises a sequence of the 3′ ABCA4 coding sequence and a sequence complementary to a portion of the 5′ ABCA4 coding sequence. In some embodiments, the first AAV vector and the second AAV vector undergo second strand synthesis to generate a first dsDNA AAV vector and a second dsDNA AAV vector. In some embodiments, the first dsDNA AAV vector and the second dsDNA AAV vector generate a full length ABCA4 transgene through homologous recombination.

Without wishing to be bound by any particular theory, a full length transgene may also be generated from an overlapping dual vector system through single-strand annealing and second strand synthesis. Upon transduction of a cell by an upstream AAV vector and a downstream AAV vector, wherein each of the upstream AAV vector and the downstream AAV vector comprises a ssDNA, and wherein the upstream AAV vector comprises a sequence encoding a 5′ portion of the transgene and the downstream AAV vector comprises a sequence encoding a 3′ portion of the transgene, the complementary upstream and downstream vectors are released into the cell or a nucleus thereof. In some embodiments, the upstream AAV vector comprises a sequence encoding a 5′ portion of the transgene and a sequence complementary to a 3′ portion of the transgene. In some embodiments, the upstream AAV vector comprises a sense sequence encoding a 5′ portion of the transgene and a sequence complementary to a 3′ portion of the transgene. In some embodiments, the upstream AAV vector comprises an antisense sequence encoding a 5′ portion of the transgene and a sequence complementary to a 3′ portion of the transgene. In some embodiments, the downstream AAV vector comprises a sequence encoding a 3′ portion of the transgene and a sequence complementary to a 5′ portion of the transgene. In some embodiments, the downstream AAV vector comprises an antisense sequence encoding a 3′ portion of the transgene and a sequence complementary to a 5′ portion of the transgene. In some embodiments, the downstream AAV vector comprises a sense sequence encoding a 3′ portion of the transgene and a sequence complementary to a 5′ portion of the transgene. In some embodiments, the upstream and downstream vectors hybridize at the region of complementarity (overlap). Following hybridization, a full length transgene is generated by second strand synthesis.

In some embodiments of the compositions and methods of the disclosure, a first AAV vector comprises a 5′ portion of an ABCA4 coding sequence, a second AAV vector comprises a 3′ portion of an ABCA4 coding sequence, and the 5′ portion and the 3′ portion overlap by at least 20 contiguous nucleotides. In some embodiments, the first AAV vector and the second AAV vector each comprise a single stranded DNA (ssDNA). In some embodiments, the first AAV vector comprises a sequence of the ABCA4 coding sequence and the second AAV vector comprises a sequence complementary to the ABCA4 coding sequence. In some embodiments, the second AAV vector comprises a sequence of the ABCA4 coding sequence and the first AAV vector comprises a sequence complementary to the ABCA4 coding sequence. In some embodiments, the first AAV vector and the second AAV vector anneal at a complementary overlapping region to generate a full length dsDNA ABCA4 transgene by subsequent second strand synthesis. In some embodiments, the full length dsDNA ABCA4 transgene is generated in vitro or in vivo (in a cell or in a subject).

The disclosure addresses the above prior art problems by providing adeno-associated viral (AAV) vector systems as described in the claims.

Dual vector approaches increase the capacity of AAV gene therapy, but may also substantially reduce levels of target protein which may be insufficient to achieve a therapeutic effect. In some embodiments of dual vector systems, the efficacy of recombination of dual vectors depends on the length of DNA overlap between the plus and minus strands (sense and antisense strands). The size of the ABCA4 coding sequence allows for the exploration of various lengths of overlap between the plus and minus strands to identify zones for optimal dual vector strategies for the treatment of disorders caused by mutations in large genes. These strategies can lead to production of enough target protein to provide therapeutic effect. In the Stargardt mouse model, therapeutic effect can be readily assessed as the target protein, ABCA4, is required in abundance in the photoreceptor cells of the retina and its absence induces the accumulation of bisretinoid compounds, which in turn leads to an increase in 790 nm autofluorescence. The therapeutic potential of the overlapping dual vector system can be validated in vivo by observing a reduction in this bisretinoid accumulation and subsequent 790 nm autofluorescence levels following treatment.

Advantageously, the AAV vector system of the disclosure provides surprisingly high levels of expression of full-length ABCA4 protein in transduced cells, with limited production of unwanted truncated fragments of ABCA4. With an optimized recombination, the full length ABCA4 protein is expressed in the photoreceptor outer segments in Abca4−/− mice and at levels sufficient to reduce bisretinoid formation and correct the autofluorescent phenotype on retinal imaging. These observations support a dual vector approach for AAV gene therapy to treat Stargardt disease.

In a first aspect, the invention provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1 or SEQ ID NO: 2; wherein the second nucleic acid sequence comprises a sequence of contiguous nucleotides corresponding to nucleotides 3806 to 6926 of SEQ ID NO: 1 or SEQ ID NO:2, wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1 or SEQ ID NO: 2.

The term “AAV vector system” is used to embrace the fact that the first and second AAV vectors are intended to work together in a complementary fashion.

The first and second AAV vectors of the AAV vector system of the invention together encode an entire ABCA4 transgene. Thus, expression of the encoded ABCA4 transgene in a target cell requires transduction of the target cell with both first (upstream) and second (downstream) vectors.

The AAV vectors of the AAV vector system of the invention are typically in the form of AAV particles (also referred to as virions). An AAV particle comprises a protein coat (the capsid) surrounding a core of nucleic acid, which is the AAV genome. The present invention also encompasses nucleic acid sequences encoding AAV vector genomes of the AAV vector system described herein.

SEQ ID NO: 1 is the human ABCA4 nucleic acid sequence corresponding to NCBI Reference Sequence NM_000350.2. SEQ ID NO: 1 is identical to NCBI Reference Sequence NM_000350.2. The ABCA4 coding sequence spans nucleotides 105 to 6926 of SEQ ID NO: 1.

SEQ ID NO: 2 is identical to SEQ ID NO: 1 with the exception of the following mutations: nucleotide 1640 G>T, nucleotide 5279 G>A, nucleotide 6173 T>C. These mutations do not alter the encoded amino acid sequence, and thus the ABCA4 protein encoded by SEQ ID NO: 2 is identical to the ABCA4 protein encoded by SEQ ID NO: 1.

In some embodiment, the first AAV vector comprises a first nucleic acid sequence comprising a 5′ end portion of an ABCA4 CDS. A 5′ end portion of an ABCA4 CDS is a portion of the ABCA4 CDS that includes its 5′ end. Because it is only a portion of a CDS, the 5′ end portion of an ABCA4 CDS is not a full-length (i.e. is not an entire) ABCA4 CDS. Thus, the first nucleic acid sequence (and thus the first AAV vector) does not comprise a full-length ABCA4 CDS.

In some embodiments, the second AAV vector comprises a second nucleic acid sequence comprising a 3′ end portion of an ABCA4 CDS. A 3′ end portion of an ABCA4 CDS is a portion of the ABCA4 CDS that includes its 3′ end. Because it is only a portion of a CDS, the 3′ end portion of an ABCA4 CDS is not a full-length (i.e. is not an entire) ABCA4 CDS. Thus, the second nucleic acid sequence (and thus the second AAV vector) does not comprise a full-length ABCA4 CDS.

The 5′ end portion and 3′ end portion together encompass the entire ABCA4 CDS (with a region of sequence overlap, as discussed below). Thus, a full-length ABCA4 CDS is contained in the AAV vector system of the invention, split across the first and second AAV vectors, and can be reassembled in a target cell following transduction of the target cell with the first and second AAV vectors.

In some embodiments, the first nucleic acid sequence as described above comprises a sequence of contiguous nucleotides corresponding to nucleotides 105 to 3597 of SEQ ID NO: 1. The ABCA4 CDS begins at nucleotide 105 of SEQ ID NO: 1 or SEQ ID NO: 2.

In some embodiments, the second nucleic acid sequence as described above comprises a sequence of contiguous nucleotides corresponding to nucleotides 3806 to 6926 of SEQ ID NO: 1 or SEQ ID NO: 2.

In order to encompass the entire ABCA4 CDS, the first and second nucleic acid sequences each further comprise at least a portion of the ABCA4 CDS corresponding to nucleotides 3598 to 3805 of SEQ ID NO: 1 or SEQ ID NO: 2, such that when the first and second nucleic acid sequences are aligned the entirety of ABCA4 CDS corresponding to nucleotides 3598 to 3805 of SEQ ID NO: 1 or SEQ ID NO: 2 is encompassed. Thus, when aligned, the first and second nucleic acid sequences together encompass the entire ABCA4 CDS.

Furthermore, the first and second nucleic acid sequences comprise a region of sequence overlap allowing reconstruction of the entire ABCA4 CDS as part of a full-length transgene inside a target cell transduced with the first and second AAV vectors of the invention.

When the first and second nucleic acid sequences are aligned with each other, a region at the 3′ end of the first nucleic acid sequence overlaps with a corresponding region at the 5′ end of the second nucleic acid sequence. Thus, both the first and second nucleic acid sequences comprise a portion of the ABCA4 CDS that forms the region of sequence overlap.

Particularly advantageous results are obtained when the region of overlap between the first and second nucleic acid sequences comprises at least about 20 contiguous nucleotides of the portion of the ABCA4 CDS corresponding to nucleotides 3598 to 3805 of SEQ ID NO: 1 or SEQ ID NO: 2.

The region of overlap may extend upstream and/or downstream of said 20 contiguous nucleotides. Thus, the region of overlap may be more than 20 nucleotides in length.

The region of overlap may comprise nucleotides upstream of the position corresponding to nucleotide 3598 of SEQ ID NO: 1 or SEQ ID NO: 2. Alternatively, or in addition, the region of overlap may comprise nucleotides downstream of the position corresponding to nucleotide 3805 of SEQ ID NO: 1 or SEQ ID NO: 2.

Alternatively, the region of nucleic acid sequence overlap may be contained within the portion of the ABCA4 CDS corresponding to nucleotides 3598 to 3805 of SEQ ID NO: 1 or SEQ ID NO: 2.

Thus, in one embodiment, the region of nucleic acid sequence overlap is between 20 and 550 nucleotides in length; preferably between 50 and 250 nucleotides in length; preferably between 175 and 225 nucleotides in length; preferably between 195 and 215 nucleotides in length.

In one embodiment, the region of nucleic acid sequence overlap comprises at least about 50 contiguous nucleotides of a nucleic acid sequence corresponding to nucleotides 3598 to 3805 of SEQ ID NO: 1 or SEQ ID NO: 2; preferably at least about 75 contiguous nucleotides; preferably at least about 100 contiguous nucleotides; preferably at least about 150 contiguous nucleotides; preferably at least about 200 contiguous nucleotides; preferably all 208 contiguous nucleotides.

In a preferred embodiment, the region of nucleic acid sequence overlap commences at the nucleotide corresponding to nucleotide 3598 of SEQ ID NO: 1 or SEQ ID NO: 2. The term “commences” means that the region of nucleic acid sequence overlap runs in the direction 5′ to 3′ starting from the nucleotide corresponding to nucleotide 3598 of SEQ ID NO: 1 or SEQ ID NO: 2. Thus, in a preferred embodiment, the most 5′ nucleotide of the region of nucleic acid sequence overlap corresponds to nucleotide 3598 of SEQ ID NO: 1 or SEQ ID NO: 2.

In a further preferred embodiment, the region of nucleic acid sequence overlap between the first nucleic acid sequence and the second nucleic acid sequence vector corresponds to nucleotides 3598 to 3805 of SEQ ID NO: 1 or SEQ ID NO: 2.

A further advantage of the present invention is that construction of dual AAV vectors comprising a region of nucleic acid sequence overlap as described above can advantageously reduce the level of translation of unwanted truncated ABCA4 peptides.

The problem of translation of truncated ABCA4 peptides may arise in dual AAV vector systems when translation is initiated from mRNA transcripts derived from the downstream vector only. In this regard, AAV ITRs such as the AAV2 5′ ITR may have promoter activity; this together with the presence in a downstream vector of WPRE and bGH poly-adenylation sequences (as discussed below) may lead to the generation of stable mRNA transcripts from unrecombined downstream vectors. The wild-type ABCA4 CDS carries multiple in-frame AUG codons in its downstream portion that cannot be substituted for other codons without altering the amino acid sequence. This creates the possibility of translation occurring from the stable transcripts, leading to the presence of truncated ABCA4 peptides.

In preferred embodiments of the invention wherein the region of nucleic acid sequence overlap commences at the nucleotide corresponding to nucleotide 3598 of SEQ ID NO: 1 or SEQ ID NO: 2, the starting sequence of the overlap zone includes an out-of-frame AUG (start) codon in good context (regarding the potential Kozak consensus sequence) prior to an in-frame AUG codon in weaker context in order to encourage the translational machinery to initiate translation of unrecombined downstream-only transcripts from an out-of-frame site. In particularly preferred embodiments of the invention, there are in total four out-of-frame AUG codons in various contexts prior to the in-frame AUG. All of these will translate to a STOP codon within 10 amino acids, thus preventing the translation of unwanted truncated ABCA4 peptides.

Preferably, the first nucleic acid sequence comprises a sequence of contiguous nucleotides corresponding to nucleotides 105 to 3805 of SEQ ID NO: 1 or SEQ ID NO:2, and the second nucleic acid sequence comprises a sequence of contiguous nucleotides corresponding to nucleotides 3598 to 6926 of SEQ ID NO: 1 or SEQ ID NO: 2, so encompassing the particularly preferred region of nucleic acid sequence overlap as described above.

Thus, in a preferred embodiment, the 5′ end portion of an ABCA4 CDS consists of a sequence of contiguous nucleotides corresponding to nucleotides 105 to 3805 of SEQ ID NO: 1 or SEQ ID NO: 2, and the 3′ end portion of an ABCA4 CDS consists of a sequence of contiguous nucleotides corresponding to nucleotides 3598 to 6926 of SEQ ID NO: 1 or SEQ ID NO: 2.

In a further preferred embodiment, the 5′ end portion of an ABCA4 CDS consists of nucleotides 105 to 3805 of SEQ ID NO: 1 or SEQ ID NO: 2, and the 3′ end portion of an ABCA4 CDS consists of nucleotides 3598 to 6926 of SEQ ID NO: 1 or SEQ ID NO: 2.

Thus, in a preferred embodiment, the invention provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence of contiguous nucleotides corresponding to nucleotides 105 to 3805 of SEQ ID NO: 1 or SEQ ID NO: 2, and wherein the 3′ end portion of an ABCA4 CDS consists of a sequence of contiguous nucleotides corresponding to nucleotides 3598 to 6926 of SEQ ID NO: 1 or SEQ ID NO: 2.

In a further preferred embodiment, the disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of nucleotides 105 to 3805 of SEQ ID NO: 1 or SEQ ID NO: 2, and wherein the 3′ end portion of an ABCA4 CDS consists of nucleotides 3598 to 6926 of SEQ ID NO: 1 or SEQ ID NO: 2.

In accordance with the term “consists of”, in embodiments wherein the 5′ end portion of an ABCA4 CDS and the 3′ end portion of an ABCA4 CDS consist of specific sequences of contiguous nucleotides as described above, then the first nucleic acid sequence and the second nucleic acid sequence each do not comprise any additional ABCA4 CDS.

Typically, each of the first AAV vector and the second AAV vector comprises 5′ and 3′ Inverted Terminal Repeats (ITRs).

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. AAV ITRs are believed to aid concatemer formation in the nucleus of an AAV-infected cell, for example following the conversion of single-stranded vector DNA into double-stranded DNA by the action of host cell DNA polymerases. The formation of such episomal concatemers may serve to protect the vector construct during the life of the host cell, thereby allowing for prolonged expression of the transgene in vivo.

Thus, in one embodiment, the ITRs are AAV ITRs (i.e. ITR sequences derived from ITR sequences found in an AAV genome).

The first and second AAV vectors of the AAV vector system of the invention together comprise all of the components necessary for a fully functional ABCA4 transgene to be re-assembled in a target cell following transduction by both vectors. A skilled person will be aware of additional genetic elements commonly used to ensure transgene expression in a viral vector-transduced cell. These may be referred to as expression control sequences. Thus, the AAV vectors of the AAV viral vector system of the invention typically comprise expression control sequences (e.g. comprising a promoter sequence) operably linked to the nucleotide sequences encoding the ABCA4 transgene.

5′ expression control sequences components are suitably located in the first (“upstream”) AAV vector of the viral vector system, while 3′ expression control sequences are suitably located in the second (“downstream”) AAV vector of the viral vector system.

Thus, the first AAV vector typically comprises a promoter operably linked to the 5′ end portion of an ABCA4 CDS. The promoter is required by its nature to be located 5′ to the ABCA4 CDS, hence its location in the first AAV vector.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, it is preferred that the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

An example promoter suitable for use in the present invention is the chicken beta-actin (CBA) promoter, optionally in combination with a cytomegalovirus (CMV) enhancer element. Another example promoter for use in the invention is a hybrid CBA/CAG promoter, for example the promoter used in the rAVE expression cassette (GeneDetect.com). Any of the promoters disclosed herein may be used.

Examples of promoters based on human sequences that would induce retina-specific gene expression include rhodopsin kinase for rods and cones, PR2.1 for cones only, and RPE65 for the retinal pigment epithelium.

### AAV-GRK1-ABCA4 Dual Vector Constructs

The present inventors have found that particularly advantageous levels of gene expression may be achieved using a GRK1 promoter. Thus, in one embodiment, the promoter is a human rhodopsin kinase (GRK1) promoter.

The GRK1 promoter sequence of the invention may be 199 nucleotides in length and comprise nucleotides −112 to +87 of the GRK1 gene. In a preferred embodiment, the promoter comprises the nucleic acid sequence of SEQ ID NO: 5 or a variant thereof having at least 90% (e.g. at least 90, 95, 96, 97, 98, 99, 99.1, 99.2, 99.3, 99.4 or 99.5, 99.6, 99.7, 99.8 or 99.9%) sequence identity.

```
(SEQ ID NO: 5)
    1 GGGCCCCAGA AGCCTGGTGG TTGTTTGTCC TTCTCAGGGG AAAAGTGAGG CGGCCCCTTG
  61 GAGGAAGGGG CCGGGCAGAA TGATCTAATC GGATTCCAAG CAGCTCAGGG GATTGTCTTT
121 TTCTAGCACC TTCTTGCCAC TCCTAAGCGT CCTCCGTGAC CCCGGCTGGG ATTTAGCCTG
181 GTGCTGTGTC AGCCCCGGG
```

The first AAV vector may comprise an untranslated region (UTR) located between the promoter and the upstream ABCA4 nucleic acid sequence (i.e. a 5′ UTR).

Any suitable UTR sequence may be used, the selection of which may be readily made by the skilled person.

The UTR may comprise one or more of the following elements: a Gallus gallus 13 actin (CBA) intron 1 fragment, an Oryctolagus cuniculus 13 globin (RBG) intron 2 fragment, and an Oryctolagus cuniculus (3 globin exon 3 fragment.

The UTR may comprise a Kozak consensus sequence. Any suitable Kozak consensus sequence may be used, the selection of which may be readily made by the skilled person.

In a preferred embodiment, the UTR comprises the nucleic acid sequence specified in SEQ ID NO: 6 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The UTR of SEQ ID NO: 6 is 186 nucleotides in length and includes a Gallus gallus 13 actin (CBA) intron 1 fragment (with predicted splice donor site), Oryctolagus cuniculus 13 globin (RBG) intron 2 fragment (including predicted branch point and splice acceptor site) and Oryctolagus cuniculus 13 globin exon 3 fragment immediately prior to a Kozak consensus sequence.

The present inventors have surprisingly found that the presence of a UTR as described above, in particular a UTR sequence as specified in SEQ ID NO: 6 or a variant thereof having at least 90% sequence identity, advantageously increases translational yield from the ABCA4 transgene.

```
(SEQ ID NO: 6)
    1 GTGCCGCAGG GGGACGGCTG CCTTCGGGGG GGACGGGGCA GGGCGGGGTT CGGCTTCTGG
  61 CGTGTGACCG GCGGCTCTAG AGCCTCTGCT AACCATGTTC ATGCCTTCTT CTTTTTCCTA
121 CAGCTCCTGG GCAACGTGCT GGTTATTGTG CTGTCTCATC ATTTTGGCAA AGAATTACCA
181 CCATGG
```

The second (“downstream”) AAV vector of the AAV vector system of the invention may comprise a post-transcriptional response element (also known as post-transcriptional regulatory element) or PRE. Any suitable PRE may be used, the selection of which may be readily made by the skilled person. The presence of a suitable PRE may enhance expression of the ABCA4 transgene.

In a preferred embodiment, the PRE is a Woodchuck Hepatitis Virus PRE (WPRE). In a particularly preferred embodiment, the WPRE has a sequence as specified in SEQ ID NO: 7 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

```
(SEQ ID NO: 7)
    1 ATCGATAATC AACCTCTGGA TTACAAAATT TGTGAAAGAT TGACTGGTAT TCTTAACTAT
  61 GTTGCTCCTT TTACGCTATG TGGATACGCT GCTTTAATGC CTTTGTATCA TGCTATTGCT
121 TCCCGTATGG CTTTCATTTT CTCCTCCTTG TATAAATCCT GGTTGCTGTC TCTTTATGAG
181 GAGTTGTGGC CCGTTGTCAG GCAACGTGGC GTGGTGTGCA CTGTGTTTGC TGACGCAACC
241 CCCACTGGTT GGGGCATTGC CACCACCTGT CAGCTCCTTT CCGGGACTTT CGCTTTCCCC
301 CTCCCTATTG CCACGGCGGA ACTCATCGCC GCCTGCCTTG CCCGCTGCTG GACAGGGGCT
361 CGGCTGTTGG GCACTGACAA TTCCGTGGTG TTGTCGGGGA AATCATCGTC CTTTCCTTGG
421 CTGCTCGCCT GTGTTGCCAC CTGGATTCTG CGCGGGACGT CCTTCTGCTA CGTCCCTTCG
481 GCCCTCAATC CAGCGGACCT TCCTTCCCGC GGCCTGCTGC CGGCTCTGCG GCCTCTTCCG
541 CGTCTTCGCC TTCGCCCTCA GACGAGTCGG ATCTCCCTTT GGGCCGCCTC CCC
```

The second AAV vector may comprise a poly-adenylation sequence located 3′ to the downstream ABCA4 nucleic acid sequence. Any suitable poly-adenylation sequence may be used, the selection of which may be readily made by the skilled person.

In a preferred embodiment, the poly-adenylation sequence is a bovine Growth Hormone (bGH) poly-adenylation sequence. In a particularly preferred embodiment, the bGH poly-adenlylation sequence has a sequence as specified in SEQ ID NO: 8 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In a preferred embodiment of the AAV vector system of the invention, the first AAV vector comprises the nucleic acid sequence of SEQ ID NO: 9, and the second AAV vector comprises the nucleic acid sequence of SEQ ID NO: 10.

In another preferred embodiment of the AAV vector system of the invention, the first AAV vector comprises the nucleic acid sequence of SEQ ID NO: 3, and the second AAV vector comprises the nucleic acid sequence of SEQ ID NO: 4.

The AAV vector system of the invention is suitable for expressing a human ABCA4 protein in a target cell.

Thus, in one aspect, the invention provides a method for expressing a human ABCA4 protein in a target cell, the method comprising the steps of: transducing the target cell with the first AAV vector and the second AAV vector as described above, such that a functional ABCA4 protein is expressed in the target cell.

Expression of human ABCA4 protein requires that the target cell be transduced with both the first AAV vector and the second AAV vector; however, the order is not important. Thus, the target cell may be transduced with the first AAV vector and the second AAV vector in any order (first AAV vector followed by second AAV vector, or second AAV vector followed by first AAV vector) or simultaneously.

Methods for transducing target cells with AAV vectors are known in the art and will be familiar to a skilled person.

The target cell is preferably a cell of the eye, preferably a retinal cell (e.g. a neuronal photoreceptor cell, a rod cell, a cone cell, or a retinal pigment epithelium cell).

The present invention also provides the first AAV vector, as defined above. There is also provided the second AAV vector, as defined above.

In another aspect, the invention provides an AAV vector, comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1. Accordingly, this AAV vector does not comprise any additional ABCA4 CDS beyond said sequence of contiguous nucleotides.

The first AAV vector may comprise 5′ and 3′ ITRs, preferably AAV ITRs; a promoter, preferably a GRK1 promoter; and/or a UTR; said elements being as described above in relation to the AAV vector system of the invention.

In one embodiment, the first AAV vector comprises the nucleic acid sequence of SEQ ID NO: 9.

In one embodiment, the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the first AAV vector comprises the nucleic acid sequence of SEQ ID NO: 9 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the first AAV vector comprises the nucleic acid sequence of SEQ ID NO: 3.

In one embodiment, the first AAV vector comprises the nucleic acid sequence of SEQ ID NO: 3 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the first AAV vector comprises the nucleic acid sequence of SEQ ID NO: 3 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In another aspect, the invention provides an AAV vector, comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1 or SEQ ID NO: 2. Accordingly, this AAV vector does not comprise any additional ABCA4 CDS beyond said sequence of contiguous nucleotides.

The second vector may comprise 5′ and 3′ ITRs, preferably AAV ITRs; a PRE, preferably a WPRE; and/or a poly-adenylation sequence, preferably a bGH poly-adenylation sequence; said elements being as described above in relation to the AAV vector system of the invention.

In one embodiment, the second AAV vector comprises the nucleic acid sequence of SEQ ID NO: 10.

In one embodiment, the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the second AAV vector comprises the nucleic acid sequence of SEQ ID NO: 10 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the second AAV vector comprises the nucleic acid sequence of SEQ ID NO: 4.

In one embodiment, the second AAV vector comprises the nucleic acid sequence of SEQ ID NO: 4 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the second AAV vector comprises the nucleic acid sequence of SEQ ID NO: 4 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The invention also provides nucleic acids comprising the nucleic acid sequences described above.

The invention also provides an AAV vector genome derivable from an AAV vector as described above.

An example AAV vector system of the invention comprises a first AAV vector and a second AAV vector; wherein the first AAV vector comprises the nucleic acid sequence of SEQ ID NO: 9; and the second AAV vector comprises the nucleic acid sequence of SEQ ID NO: 10.

A further example AAV vector system of the invention comprises a first AAV vector and a second AAV vector; wherein the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity; and the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In particular embodiments, the methods and compositions disclosed herein relate to any of the following vectors: CMVCBA.In.GFP.pA vector (SEQ ID NO: 17); CMVCBA.GFP.pA vector (SEQ ID NO: 18); CBA.IntEx.GFP.pA vector (SEQ ID NO: 19); CAG.GFP.pA vector (SEQ ID NO: 20); AAV.5′CMVCBA.In.ABCA4.WPRE.kan vector (SEQ ID NO: 21); AAV.5′CMVCBA.ABCA4.WPRE.kan vector (SEQ ID NO: 22); or AAV.5′CBA.IntEx.ABCA4.WPRE.kan vector (SEQ ID NO: 23).

In particular embodiments, the methods and compositions disclosed herein are directed to any of the following sequences: (i) the ITR to ITR portion of pAAV.RK.5′ABCA4.kan (SEQ ID NO: 26), comprising a sequence encoding a 5′ ITR (SEQ ID NO: 27), a sequence encoding an RK promoter (SEQ ID NO: 28), a sequence encoding a Rabbit Beta-Globin (RBG) Intron/Exon (Int/Ex) (SEQ ID NO: 39), a sequence encoding a 5′ portion of the coding sequence of an ABCA4 gene (SEQ ID NO: 29), and a sequence encoding a 3′ ITR (SEQ ID NO: 30); or (ii) a sequence of the ITR to ITR portion of pAAV.3′ABCA4.WPRE.kan (SEQ ID NO: 30), comprising a sequence encoding a 5′ ITR (SEQ ID NO: 27), a sequence encoding a 3′ portion of the coding sequence of an ABCA4 gene (SEQ ID NO: 31), a sequence encoding WPRE (SEQ ID NO: 32), a sequence encoding bGH polyA and a sequence encoding a 3′ ITR (SEQ ID NO: 33).

The present invention may also be performed where SEQ ID NO: 2 is used as a reference sequence in place of SEQ ID NO: 1.

In this regard, SEQ ID NO: 2 is identical to SEQ ID NO: 1 with the exception of the following mutations: nucleotide 1640 G>T, nucleotide 5279 G>A, nucleotide 6173 T>C. These mutations do not alter the encoded amino acid sequence, and thus the ABCA4 protein encoded by SEQ ID NO: 2 is identical to the ABCA4 protein encoded by SEQ ID NO: 1.

Thus, in alternative embodiments of the invention, references above to SEQ ID NO: 1 may be replaced with references to SEQ ID NO: 2. In addition, any of the constructs disclosed herein may alternatively comprise a different promoter, such as, e.g., a CMV.CBA promoter, a CBA.RBG promoter, or a CBA.InEx promoter. Similarly, any of the constructs may comprises a 5′ ITR comprising or consisting of SEQ ID NO: 6 and/or a 3′ ITR comprising or consisting of SEQ ID NO: 37.

Sequence Correspondence

As used herein, the term “corresponding to” when used with regard to the nucleotides in a given nucleic acid sequence defines nucleotide positions by reference to a particular SEQ ID NO. However, when such references are made, it will be understood that the invention is not to be limited to the exact sequence as set out in the particular SEQ ID NO referred to but includes variant sequences thereof. The nucleotides corresponding to the nucleotide positions in SEQ ID NO: 1 can be readily determined by sequence alignment, such as by using sequence alignment programs, the use of which is well known in the art. In this regard, a skilled person would readily appreciate that the degenerate nature of the genetic code means that variations in a nucleic acid sequence encoding a given polypeptide may be present without changing the amino acid sequence of the encoded polypeptide. Thus, identification of nucleotide locations in other ABCA4 coding sequences is contemplated (i.e. nucleotides at positions which the skilled person would consider correspond to the positions identified in, for example, SEQ ID NO: 1).

By way of example, SEQ ID NO: 2 is identical to SEQ ID NO: 1 with the exception of three specific mutations, as described above (these three mutations do not alter the amino acid sequence of the encoded ABCA4 polypeptide). In this case, a skilled person would therefore consider that a given nucleotide position in SEQ ID NO: 2 corresponded to the equivalent numbered nucleotide position in SEQ ID NO: 1.

Typically, a derivative of an AAV genome will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

AAV vectors of the disclosure include transcapsidated forms wherein an AAV genome or derivative having an ITR of one serotype is packaged in the capsid of a different serotype. AAV vectors of the invention also include mosaic forms wherein a mixture of unmodified capsid proteins from two or more different serotypes makes up the viral capsid. An AAV vector may also include chemically modified forms bearing ligands adsorbed to the capsid surface. For example, such ligands may include antibodies for targeting a particular cell surface receptor.

Thus, for example, AAV vectors of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8).

An AAV vector of the invention may comprise a mutant AAV capsid protein. In one embodiment, an AAV vector of the invention comprises a mutant AAV8 capsid protein. Preferably the mutant AAV8 capsid protein is an AAV8 Y733F capsid protein.

### AAV-CBA-ABCA4 Dual Vector Constructs

The disclosure provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1 or SEQ ID NO: 2; wherein the second nucleic acid sequence comprises a sequence of contiguous nucleotides corresponding to nucleotides 3806 to 6926 of SEQ ID NO: 1 or SEQ ID NO: 2; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1 or SEQ ID NO: 2.

AAV vectors in general are well known in the art and a skilled person is familiar with general techniques suitable for their preparation from his common general knowledge in the field. The skilled person's knowledge includes techniques suitable for incorporating a nucleic acid sequence of interest into the genome of an AAV vector.

The term “AAV vector system” is used to embrace the fact that the first and second AAV vectors are intended to work together in a complementary fashion.

The first and second AAV vectors of the AAV vector system of the disclosure together encode an entire ABCA4 transgene. Thus, expression of the encoded ABCA4 transgene in a target cell requires transduction of the target cell with both first (upstream) and second (downstream) vectors.

The AAV vectors of the AAV vector system of the disclosure can be in the form of AAV particles (also referred to as virions). An AAV particle comprises a protein coat (the capsid) surrounding a core of nucleic acid, which is the AAV genome. The present disclosure also encompasses nucleic acid sequences encoding AAV vector genomes of the AAV vector system described herein.

SEQ ID NO: 1 is the human ABCA4 nucleic acid sequence corresponding to NCBI Reference Sequence NM_000350.2. SEQ ID NO: 1 is identical to NCBI Reference Sequence NM_000350.2. The ABCA4 coding sequence spans nucleotides 105 to 6926 of SEQ ID NO: 1 or SEQ ID NO: 2.

The first AAV vector comprises a first nucleic acid sequence comprising a 5′ end portion of an ABCA4 CDS. A 5′ end portion of an ABCA4 CDS is a portion of the ABCA4 CDS that includes its 5′ end. Because it is only a portion of a CDS, the 5′ end portion of an ABCA4 CDS is not a full-length (i.e. is not an entire) ABCA4 CDS. Thus, the first nucleic acid sequence (and thus the first AAV vector) does not comprise a full-length ABCA4 CDS.

The second AAV vector comprises a second nucleic acid sequence comprising a 3′ end portion of an ABCA4 CDS. A 3′ end portion of an ABCA4 CDS is a portion of the ABCA4 CDS that includes its 3′ end. Because it is only a portion of a CDS, the 3′ end portion of an ABCA4 CDS is not a full-length (i.e. is not an entire) ABCA4 CDS. Thus, the second nucleic acid sequence (and thus the second AAV vector) does not comprise a full-length ABCA4 CDS.

The 5′ end portion and 3′ end portion together encompass the entire ABCA4 CDS (with a region of sequence overlap, as discussed below). Thus, a full-length ABCA4 CDS is contained in the AAV vector system of the disclosure, split across the first and second AAV vectors, and can be reassembled in a target cell following transduction of the target cell with the first and second AAV vectors.

The first nucleic acid sequence as described above comprises a sequence of contiguous nucleotides corresponding to nucleotides 105 to 3597 of SEQ ID NO: 1 or SEQ ID NO: 2. The ABCA4 CDS begins at nucleotide 105 of SEQ ID NO: 1 or SEQ ID NO: 2.

The second nucleic acid sequence as described above comprises a sequence of contiguous nucleotides corresponding to nucleotides 3806 to 6926 of SEQ ID NO: 1 or SEQ ID NO: 2.

In order to encompass the entire ABCA4 CDS, the first and second nucleic acid sequences each further comprise at least a portion of the ABCA4 CDS corresponding to nucleotides 3598 to 3805 of SEQ ID NO: 1 or SEQ ID NO: 2, such that when the first and second nucleic acid sequences are aligned the entirety of ABCA4 CDS corresponding to nucleotides 3598 to 3805 of SEQ ID NO: 1 or SEQ ID NO: 2 is encompassed. Thus, when aligned, the first and second nucleic acid sequences together encompass the entire ABCA4 CDS.

Furthermore, the first and second nucleic acid sequences comprise a region of sequence overlap allowing reconstruction of the entire ABCA4 CDS as part of a full-length transgene inside a target cell transduced with the first and second AAV vectors of the disclosure.

When the first and second nucleic acid sequences are aligned with each other, a region at the 3′ end of the first nucleic acid sequence overlaps with a corresponding region at the 5′ end of the second nucleic acid sequence. Thus, both the first and second nucleic acid sequences comprise a portion of the ABCA4 CDS that forms the region of sequence overlap.

In some embodiments, the region of overlap between the first and second nucleic acid sequences comprises at least about 20 contiguous nucleotides of the portion of the ABCA4 CDS corresponding to nucleotides 3598 to 3805 of SEQ ID NO: 1 or SEQ ID NO: 2.

In some embodiments, the region of overlap may extend upstream and/or downstream of said 20 contiguous nucleotides. Thus, the region of overlap may be more than 20 nucleotides in length.

The region of overlap may comprise nucleotides upstream of the position corresponding to nucleotide 3598 of SEQ ID NO: 1 or SEQ ID NO: 2. Alternatively, or in addition, the region of overlap may comprise nucleotides downstream of the position corresponding to nucleotide 3805 of SEQ ID NO: 1 or SEQ ID NO: 2.

Alternatively, the region of nucleic acid sequence overlap may be contained within the portion of the ABCA4 CDS corresponding to nucleotides 3598 to 3805 of SEQ ID NO: 1 or SEQ ID NO: 2.

Thus, in one embodiment, the region of nucleic acid sequence overlap is between 20 and 550 nucleotides in length; preferably between 50 and 250 nucleotides in length; preferably between 175 and 225 nucleotides in length; preferably between 195 and 215 nucleotides in length.

In one embodiment, the region of nucleic acid sequence overlap comprises at least about 50 contiguous nucleotides of a nucleic acid sequence corresponding to nucleotides 3598 to 3805 of SEQ ID NO: 1 or SEQ ID NO: 2; preferably at least about 75 contiguous nucleotides; preferably at least about 100 contiguous nucleotides; preferably at least about 150 contiguous nucleotides; preferably at least about 200 contiguous nucleotides; preferably all 208 contiguous nucleotides.

In certain preferred embodiments, the region of nucleic acid sequence overlap commences at the nucleotide corresponding to nucleotide 3598 of SEQ ID NO: 1 or SEQ ID NO: 2. The term “commences” means that the region of nucleic acid sequence overlap runs in the direction 5′ to 3′ starting from the nucleotide corresponding to nucleotide 3598 of SEQ ID NO: 1 or SEQ ID NO: 2. Thus, in a preferred embodiment, the most 5′ nucleotide of the region of nucleic acid sequence overlap corresponds to nucleotide 3598 of SEQ ID NO: 1 or SEQ ID NO: 2.

In certain preferred embodiments, the region of nucleic acid sequence overlap between the first nucleic acid sequence and the second nucleic acid sequence vector corresponds to nucleotides 3598 to 3805 of SEQ ID NO: 1 or SEQ ID NO: 2.

A construction of dual AAV vectors comprising a region of nucleic acid sequence overlap as described above can reduce the level of translation of unwanted truncated ABCA4 peptides.

The problem of translation of truncated ABCA4 peptides may arise in dual AAV vector systems when translation is initiated from mRNA transcripts derived from the downstream vector only. In this regard, AAV ITRs such as the AAV2 5′ ITR may have promoter activity; this together with the presence in a downstream vector of WPRE and bGH poly-adenylation sequences (as discussed below) may lead to the generation of stable mRNA transcripts from unrecombined downstream vectors. The wild-type ABCA4 CDS carries multiple in-frame AUG codons in its downstream portion that cannot be substituted for other codons without altering the amino acid sequence. This creates the possibility of translation occurring from the stable transcripts, leading to the presence of truncated ABCA4 peptides.

In certain preferred embodiments of the disclosure wherein the region of nucleic acid sequence overlap commences at the nucleotide corresponding to nucleotide 3598 of SEQ ID NO: 1, the starting sequence of the overlap zone includes an out-of-frame AUG (start) codon in good context (regarding the potential Kozak consensus sequence) prior to an in-frame AUG codon in weaker context in order to encourage the translational machinery to initiate translation of unrecombined downstream-only transcripts from an out-of-frame site. In certain particularly preferred embodiments of the disclosure, there are in total four out-of-frame AUG codons in various contexts prior to the in-frame AUG. All of these translate to a STOP codon within 10 amino acids, thus preventing the translation of unwanted truncated ABCA4 peptides.

In certain preferred embodiments, the first nucleic acid sequence comprises a sequence of contiguous nucleotides corresponding to nucleotides 105 to 3805 of SEQ ID NO: 1, and the second nucleic acid sequence comprises a sequence of contiguous nucleotides corresponding to nucleotides 3598 to 6926 of SEQ ID NO: 1 or SEQ ID NO: 2, so encompassing the region of nucleic acid sequence overlap as described above.

Thus, in certain preferred embodiments, the 5′ end portion of an ABCA4 CDS consists of a sequence of contiguous nucleotides corresponding to nucleotides 105 to 3805 of SEQ ID NO: 1 or SEQ ID NO: 2, and the 3′ end portion of an ABCA4 CDS consists of a sequence of contiguous nucleotides corresponding to nucleotides 3598 to 6926 of SEQ ID NO: 1 or SEQ ID NO: 2.

In certain preferred embodiments, the 5′ end portion of an ABCA4 CDS consists of nucleotides 105 to 3805 of SEQ ID NO: 1 or SEQ ID NO: 2, and the 3′ end portion of an ABCA4 CDS consists of nucleotides 3598 to 6926 of SEQ ID NO: 1 or SEQ ID NO: 2.

Thus, in certain preferred embodiments, the disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence of contiguous nucleotides corresponding to nucleotides 105 to 3805 of SEQ ID NO: 1 or SEQ ID NO: 2, and wherein the 3′ end portion of an ABCA4 CDS consists of a sequence of contiguous nucleotides corresponding to nucleotides 3598 to 6926 of SEQ ID NO: 1 or SEQ ID NO: 2.

In certain preferred embodiments, the disclosure provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of nucleotides 105 to 3805 of SEQ ID NO: 1 or SEQ ID NO: 2, and wherein the 3′ end portion of an ABCA4 CDS consists of nucleotides 3598 to 6926 of SEQ ID NO: 1 or SEQ ID NO: 2.

In accordance with the term “consists of”, in embodiments wherein the 5′ end portion of an ABCA4 CDS and the 3′ end portion of an ABCA4 CDS consist of specific sequences of contiguous nucleotides as described above, then the first nucleic acid sequence and the second nucleic acid sequence each do not comprise any additional ABCA4 CDS.

In certain embodiments, each of the first AAV vector and the second AAV vector comprises 5′ and 3′ Inverted Terminal Repeats (ITRs).

In certain embodiments, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. AAV ITRs are believed to aid concatemer formation in the nucleus of an AAV-infected cell, for example following the conversion of single-stranded vector DNA into double-stranded DNA by the action of host cell DNA polymerases. The formation of such episomal concatemers may serve to protect the vector construct during the life of the host cell, thereby allowing for prolonged expression of the transgene in vivo.

Thus, in some embodiments, the ITRs are AAV ITRs (i.e. ITR sequences derived from ITR sequences found in an AAV genome).

The first and second AAV vectors of the AAV vector system of the disclosure together comprise all of the components necessary for a fully functional ABCA4 transgene to be re-assembled in a target cell following transduction by both vectors. A skilled person is aware of additional genetic elements commonly used to ensure transgene expression in a viral vector-transduced cell. These may be referred to as expression control sequences. Thus, the AAV vectors of the AAV viral vector system of the disclosure may comprise expression control sequences (e.g. comprising a promoter sequence) operably linked to the nucleotide sequences encoding the ABCA4 transgene.

5′ expression control sequences components can be located in the first (“upstream”) AAV vector of the viral vector system, while 3′ expression control sequences can be located in the second (“downstream”) AAV vector of the viral vector system.

Thus, in some embodiments, the first AAV vector may comprise a promoter operably linked to the 5′ end portion of an ABCA4 CDS. The promoter may be required by its nature to be located 5′ to the ABCA4 CDS, hence its location in the first AAV vector.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In those embodiments where the vector is administered for therapy, the promoter should be functional in the target cell background.

In some embodiments, the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

Elements may be included in both the upstream and downstream vectors of the disclosure to increase expression of ABCA4 protein. For example, the inclusion of an intron in a vector, such as the upstream vector of the disclosure, can increase the expression of an RNA or protein of interest from that vector. An intron is a nucleotide sequence within a gene that is removed by RNA splicing during RNA maturation. Introns can vary in length from tens of base pairs to multiple megabases. However, spliceosomal introns (i.e. introns that are spliced by the eukaryotic spliceosome) may comprise a splice donor (SD) site at the 5′ end of the intron, a branch site in the intron near the 3′ end, and a splice acceptor (SA) site at the 3′ end. These intron elements facilitate proper intron splicing. SD sites may comprise a consensus GU at the 5′ end of the intron and the SA site at the 3′ end of the intron may terminate with “AG.” Upstream of the SA site, introns often contain a region high in pyrimidines, which is between the branch point adenine nucleotide and the SA. Without wishing to be bound by any particular theory, the presence of an intron can affect the rate of RNA transcription, nuclear export or RNA transcript stability. Further, the presence of an intron may also increase the efficiency of mRNA translation, yielding more of a protein of interest (e.g. ABCA4). FIGS. 309 and 310 describe two exemplary introns (and accompanying exons) for use with ABCA4 dual vectors, IntEx and RBG SA/SD. However, the disclosure encompasses the use in a construct of the disclosure any intron that boosts gene expression and facilitates splicing in a eukaryotic cell.

In some embodiments of the vectors of the disclosure, the intron, the IntEx or the SA/SD (including a RBD SA/SD) may be one of several elements that function to increase protein expression from the vector. For example, the promoter and, optionally, an enhancer, can affect not just cell or tissue specificity of gene expression, but also the levels of mRNA that are transcribed from the vector. Promoters are regions of DNA that initiate RNA transcription. Depending on the specific sequence elements of the promoter, promoters may vary in strength and tissue specificity. Enhancers are DNA sequences that regulate transcription from promoters by affecting the ability of the promoter to recruit RNA polymerase and initiate transcription. Therefore, the choice of promoter, and optionally, the inclusion of an enhancer and/or the choice of the enhancer itself, in a vector can significantly affect the expression of a gene encoded by the vector. Exemplary promoters, such as the rhodopsin kinase promoter or chicken beta actin promoter, optionally combined with a CMV enhancer, are shown in FIGS. 310 and 311. In some embodiments, vectors of the disclosure comprise an exemplary promoter, such as the rhodopsin kinase promoter or chicken beta actin promoter, while excluding the use of an enhancer element. In some embodiments, vectors of the disclosure comprise an exemplary promoter, such as the chicken beta actin promoter, while excluding the use of an enhancer element, such as a CMV enhancer element. In some embodiments, vectors of the disclosure comprise an exemplary promoter, such as the rhodopsin kinase promoter or chicken beta actin promoter, while excluding the use of an enhancer element and while including an intron, an IntEx or an SD/SA. In some embodiments, vectors of the disclosure comprise an exemplary promoter, such as the chicken beta actin promoter, while excluding the use of an enhancer element, such as a CMV enhancer element and while including an intron, an IntEx or an SD/SA.

Elements in the non-coding sequences of the mRNA transcript itself can also affect protein levels of a sequence encoded in a vector. Without wishing to be limited by any particular theory, sequence elements in the mRNA untranslated regions (UTRs) can effect mRNA stability, which, in turn, affects levels of protein translation. An exemplary sequence element is a Posttranscriptional Regulatory Element (PRE) (e.g. a Woodchuck Hepatitis PRE (WPRE)), which increases mRNA stability. Exemplary promoters, enhancers, PREs, and the arrangement of these elements in vectors of the disclosure, are shown in FIGS. 307-316.

In some embodiments of the first AAV vector of the disclosure, the promoter may be operably linked with an intron and an exon sequence. In some embodiments of the first AAV vector of the disclosure, a nucleic acid sequence may comprise the promoter, an intron and an exon sequence. The intron and the exon sequence may be downstream of the promoter sequence. The intron and the exon sequence may be positioned between the promoter sequence and the upstream ABCA4 nucleic acid sequence (US-ABCA4). The presence of an intron and an exon may increase levels of protein expression. In some embodiments, the intron is positioned between the promoter and the exon. In some embodiments, including those embodiments wherein the intron is positioned between the promoter and the exon, the exon is positioned 5′ of the US-ABCA4 sequence. In some embodiments, the promoter comprises a promoter isolated or derived from a vertebrate gene. In some embodiments, the promoter is GRK1 promoter or a chicken beta actin (CBA) promoter. In some embodiments, the promoter is a CMV.CBA promoter, a CBA.RGB promoter, or a CBA.InEx promoter.

The exon may comprise a coding sequence, a non-coding sequence, or a combination of both. In some embodiments, the exon comprises a non-coding sequence. In some embodiments, the exon is isolated or derived from a mammalian gene. In embodiments, the mammal is a rabbit (Oryctolagus cuniculus). In some embodiments, the mammalian gene comprises a rabbit beta globin gene or a portion thereof. In some embodiments, the exon comprises or consists of a nucleic acid sequence having at least 80%, at least 90%, at least 95%, at least 96%, at least 97%, at least 98%, or at least 99% identity to the nucleic acid sequence of: CTCCTGGGCA ACGTGCTGGT TATTGTGCTG TCTCATCATT TTGGCAAAGA ATT (SEQ ID NO: 14).

In some embodiments, the exon comprises or consists of a nucleic acid sequence having 100% identity to the nucleic acid sequence of:

(SEQ ID NO: 14)
CTCCTGGGCA ACGTGCTGGT TATTGTGCTG TCTCATCATT
TTGGCAAAGA ATT.

Introns may comprise a splice donor site, a splice acceptor site or a branch point. Introns may comprise a splice donor site, a splice acceptor site and a branch point. Exemplary splice acceptor sites comprise nucleotides “GT” (“GU” in the pre-mRNA) at the 5′ end of the intron. Exemplary splice acceptor sites comprise an “AG” at the 3′ end of the intron. In some embodiments, the branch point comprises an adenosine (A) between 20 and 40 nucleotides, inclusive of the endpoints, upstream of the 3′ end of the intron. The intron may comprise an artificial or non-naturally occurring sequence. Alternatively, the intron may be isolated or derived from a vertebrate gene. The intron may comprise a sequence encoding a fusion of two sequences, each of which may be isolated or derived from a vertebrate gene. In some embodiments, a vertebrate gene from which the intron nucleic acid sequence or a portion thereof is derived comprises a chicken (Gallus gallus) gene. In some embodiments, the chicken gene comprises a chicken beta actin gene. In some embodiments, a vertebrate gene from which the intron nucleic acid sequence or a portion thereof is derived comprises a rabbit (Oryctolagus cuniculus) gene. In some embodiments, the rabbit gene comprises a rabbit beta globin gene or a portion thereof. In some embodiments, the intron comprises or consists of a nucleic acid sequence having at least 80%, at least 90%, at least 95%, at least 96%, at least 97%, at least 98%, or at least 99% identity to the nucleic acid sequence of:

```
(SEQ ID NO: 13)
    1 GTGCCGCAGG GGGACGGCTG CCTTCGGGGG GGACGGGGCA GGGCGGGGTT CGGCTTCTGG
  61 CGTGTGACCG GCGGCTCTAG AGCCTCTGCT AACCATGTTC ATGCCTTCTT CTTTTTCCTA
121 CAG.
```

In some embodiments, the intron comprises or consists of a nucleic acid sequence having 100% identity to the nucleic acid sequence of:

```
(SEQ ID NO: 13)
    1 GTGCCGCAGG GGGACGGCTG CCTTCGGGGG GGACGGGGCA GGGCGGGGTT CGGCTTCTGG
  61 CGTGTGACCG GCGGCTCTAG AGCCTCTGCT AACCATGTTC ATGCCTTCTT CTTTTTCCTA
121 CAG.
```

In some embodiments of the first (or upstream) AAV vector, the promoter comprises a hybrid promoter (a Cytomegalovirus (CMV) enhancer with a chicken beta actin (CBA) promoter). In some embodiments, the CMV enhancer sequence comprises or consists of a nucleic acid sequence having at least 80%, at least 90%, at least 95%, at least 96%, at least 97%, at least 98%, at least 99%, or at least any percentage identity in between to the nucleic acid sequence of:

```
(SEQ ID NO: 15)
    1 CCATTGACGT CAATAATGAC GTATGTTCCC ATAGTAACGC CAATAGGGAC TTTCCATTGA
  61 CGTCAATGGG TGGAGTATTT ACGGTAAACT GCCCACTTGG CAGTACATCA AGTGTATCAT
121 ATGCCAAGTA CGCCCCCTAT TGACGTCAAT GACGGTAAAT GGCCCGCCTG GCATTATGCC
181 CAGTACATGA CCTTATGGGA CTTTCCTACT TGGCAGTACA TCTACGTATT AGTCA.
```

In some embodiments, the sequence encoding the first (or upstream) AAV vector comprises a sequence encoding a CBA promoter (without a CMV enhancer element), a sequence encoding an intron and a sequence encoding an exon. In some embodiments, the CBA promoter sequence comprises or consists of a nucleic acid sequence having at least 80%, at least 90%, at least 95%, at least 96%, at least 97%, at least 98%, at least 99%, or at least any percentage identity in between to the nucleic acid sequence of:

```
(SEQ ID NO: 16)
    1 GTCGAGGTGA GCCCCACGTT CTGCTTCACT CTCCCCATCT CCCCCCCCTC CCCACCCCCA
  61 ATTTTGTATT TATTTATTTT TTAATTATTT TGTGCAGCGA TGGGGGCGGG GGGGGGGGGG
121 GGGCGCGCGC CAGGCGGGGC GGGGCGGGGC GAGGGGCGGG GCGGGGCGAG GCGGAGAGGT
181 GCGGCGGCAG CCAATCAGAG CGGCGCGCTC CGAAAGTTTC CTTTTATGGC GAGGCGGCGG
241 CGGCGGCGGC CCTATAAAAA GCGAAGCGCG CGGCGGGCGG GAGTCGCTGC GCGCTGCCTT
301 CGCCCCGTGC CCCGCTCCGC CGCCGCCTCG CGCCGCCCGC CCCGGCTCTG ACTGACCGCG
361 TTACTCCCAC AG.
```

In some embodiments, the CBA promoter sequence comprises or consists of a nucleic acid sequence having at least 80%, at least 90%, at least 95%, at least 96%, at least 97%, at least 98%, at least 99%, or at least any percentage identity in between to the nucleic acid sequence of:

```
(SEQ ID NO: 24)
    1 GTCGAGGTGA GCCCCACGTT CTGCTTCACT CTCCCCATCT CCCCCCCCTC CCCACCCCCA
  61 ATTTTGTATT TATTTATTTT TTAATTATTT TGTGCAGCGA TGGGGGCGGG GGGGGGGGGG
121 GGGCGCGCGC CAGGCGGGGC GGGGCGGGGC GAGGGGCGGG GCGGGGCGAG GCGGAGAGGT
181 GCGGCGGCAG CCAATCAGAG CGGCGCGCTC CGAAAGTTTC CTTTTATGGC GAGGCGGCGG
241 CGGCGGCGGC CCTATAAAAA GCGAAGCGCG CGGCGGGCG.
```

In some embodiments, the sequence encoding the intron comprises or consists of the nucleic acid sequence of SEQ ID NO: 13. In some embodiments, the sequence encoding the exon comprises or consists of the nucleic acid sequence of SEQ ID NO: 14.

The first AAV vector may comprise an untranslated region (UTR) located between the promoter and the upstream ABCA4 nucleic acid sequence (i.e. a 5′ UTR).

Any suitable UTR sequence may be used, the selection of which may be readily made by the skilled person.

The UTR may comprise or consist of one or more of the following elements: a Gallus β-actin (CBA) intron 1 or a portion thereof, an Oryctolagus cuniculus β-globin (RBG) intron 2 or a portion thereof, and an Oryctolagus cuniculus β-globin exon 3 or a portion thereof.

The UTR may comprise a Kozak consensus sequence. Any suitable Kozak consensus sequence may be used.

In certain preferred embodiments, the UTR comprises the nucleic acid sequence specified in SEQ ID NO: 6, a variant or a portion thereof having at least 90% (e.g. at least 90%, 95%, 96%, 97%, 98%, 99%, 99.1%, 99.2%, 99.3%, 99.4%, 99.5%, 99.6%, 99.7%, 99.8% or 99.9%) sequence identity.

The UTR of SEQ ID NO: 6 is 186 nucleotides in length and includes a Gallus β-actin (CBA) intron 1 fragment (with predicted splice donor site), Oryctolagus cuniculus β-globin (RBG) intron 2 fragment (including predicted branch point and splice acceptor site) and Oryctolagus cuniculus β-globin exon 3 fragment immediately prior to a Kozak consensus sequence.

The presence of a UTR as described above, in particular a UTR sequence as specified in SEQ ID NO: 6 or a variant thereof having at least 90% sequence identity, may increase translational yield from the ABCA4 transgene.

The second (“downstream”) AAV vector of the AAV vector system of the disclosure may comprise a post-transcriptional response element (also known as post-transcriptional regulatory element) or PRE. Any suitable PRE may be used, the selection of which may be readily made by the skilled person. In certain embodiments, the presence of a suitable PRE may enhance expression of the ABCA4 transgene.

In certain preferred embodiments, the PRE is a Woodchuck Hepatitis Virus PRE (WPRE). In certain particularly preferred embodiments, the WPRE has a sequence as specified in SEQ ID NO: 7 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The second AAV vector may comprise a poly-adenylation sequence located 3′ to the downstream ABCA4 nucleic acid sequence. Any suitable poly-adenylation sequence may be used, the selection of which may be readily made by the skilled person.

In certain preferred embodiments, the poly-adenylation sequence is a bovine Growth Hormone (bGH) poly-adenylation sequence. In a particularly preferred embodiment, the bGH poly-adenlylation sequence has a sequence as specified in SEQ ID NO: 8 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity. In certain embodiments, the sequence encoding the polyadenylation sequence comprises or consists of a nucleic acid sequence having at least 80%, at least 90%, at least 95%, at least 96%, at least 97%, at least 98%, at least 99%, or at least any percentage identity in between to the nucleic acid sequence of:

```
(SEQ ID NO: 25)
    1 CGCTGATCAG CCTCGACTGT GCCTTCTAGT TGCCAGCCAT CTGTTGTTTG CCCCTCCCCC
  61 GTGCCTTCCT TGACCCTGGA AGGTGCCACT CCCACTGTCC TTTCCTAATA AAATGAGGAA
121 ATTGCATCGC ATTGTCTGAG TAGGTGTCAT TCTATTCTGG GGGGTGGGGT GGGGCAGGAC
181 AGCAAGGGGG AGGATTGGGA AGACAATAGC AGGCATGCTG GGGATGCGGT GGGCTCTATG
241 GCTTCTGAGG CGGAAAGAAC CAG.
```

In certain preferred embodiments of the AAV vector system of the disclosure, the first AAV vector comprises the nucleic acid sequence of SEQ ID NO: 9, and the second AAV vector comprises the nucleic acid sequence of SEQ ID NO: 10.

In certain preferred embodiments of the AAV vector system of the disclosure, the first AAV vector comprises the nucleic acid sequence of SEQ ID NO: 3, and the second AAV vector comprises the nucleic acid sequence of SEQ ID NO: 4.

The AAV vector system of the disclosure may be suitable for expressing a human ABCA4 protein in a target cell.

The disclosure provides a method for expressing a human ABCA4 protein in a target cell, the method comprising the steps of: transducing the target cell with the first AAV vector and the second AAV vector as described above, such that a functional ABCA4 protein is expressed in the target cell.

Expression of human ABCA4 protein requires that the target cell be transduced with both the first AAV vector and the second AAV vector. In certain embodiments, the target cell may be transduced with the first AAV vector and the second AAV vector in any order (first AAV vector followed by second AAV vector, or second AAV vector followed by first AAV vector) or simultaneously.

Methods for transducing target cells with AAV vectors are known in the art and will be familiar to a skilled person.

The target cell is may be a cell of the eye, preferably a retinal cell (e.g. a neuronal photoreceptor cell, a rod cell, a cone cell, or a retinal pigment epithelium cell).

The disclosure also provides the first AAV vector, as defined above. There is also provided the second AAV vector, as defined above.

The disclosure provides an AAV vector, comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1. In certain embodiments, this AAV vector does not comprise any additional ABCA4 CDS beyond said sequence of contiguous nucleotides.

The first AAV vector may comprise 5′ and 3′ ITRs, preferably AAV ITRs; a promoter, for example a GRK1 promoter; and/or a UTR; said elements being as described above in relation to the AAV vector system of the disclosure. In some embodiments, the promoter is a CMV.CBA promoter, a CBA.RGB promoter, or a CBA.InEx promoter.

In some embodiments, the first AAV vector comprises the nucleic acid sequence of SEQ ID NO: 9.

In some embodiments, the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the first AAV vector comprises the nucleic acid sequence of SEQ ID NO: 9 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the first AAV vector comprises the nucleic acid sequence of SEQ ID NO: 3.

In some embodiments, the first AAV vector comprises the nucleic acid sequence of SEQ ID NO: 3 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the first AAV vector comprises the nucleic acid sequence of SEQ ID NO: 3 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The disclosure provides an AAV vector, comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1 or SEQ ID NO: 2. In some embodiments, this AAV vector does not comprise any additional ABCA4 CDS beyond said sequence of contiguous nucleotides.

The second vector may comprise 5′ and 3′ ITRs, preferably AAV ITRs; a PRE, preferably a WPRE; and/or a poly-adenylation sequence, preferably a bGH poly-adenylation sequence; said elements being as described above in relation to the AAV vector system of the disclosure.

In some embodiments, the second AAV vector comprises the nucleic acid sequence of SEQ ID NO: 10.

In some embodiments, the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the second AAV vector comprises the nucleic acid sequence of SEQ ID NO: 10 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the second AAV vector comprises the nucleic acid sequence of SEQ ID NO: 4.

In some embodiments, the second AAV vector comprises the nucleic acid sequence of SEQ ID NO: 4 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the second AAV vector comprises the nucleic acid sequence of SEQ ID NO: 4 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The disclosure also provides nucleic acids comprising the nucleic acid sequences described above. The disclosure also provides an AAV vector genome derivable from an AAV vector as described above.

Also provided is a kit comprising the first AAV vector and the second AAV vector as described above. The AAV vectors may be provided in the kits in the form of AAV particles.

Further provided is a kit comprising a nucleic acid comprising the first nucleic acid sequence and a nucleic acid comprising the second nucleic acid sequence, as described above.

The disclosure also provides a pharmaceutical composition comprising the AAV vector system as described above and a pharmaceutically acceptable excipient.

The AAV vector system of the disclosure, the kit of the disclosure, and the pharmaceutical composition of the disclosure, may be used in gene therapy. For example, AAV vector system of the disclosure, the kit of the disclosure, and the pharmaceutical composition of the disclosure, may be used in preventing or treating disease.

In some embodiments, use of the compositions and methods of the disclosure to prevent or treat disease comprises administration of the first AAV vector and second AAV vector to a target cell, to provide expression of ABCA4 protein.

In some embodiments, the disease to be prevented or treated is characterized by degradation of retinal cells. An example of such a disease is Stargardt disease. In some embodiments, the first and second AAV vectors of the disclosure may be administered to an eye of a patient, for example to retinal tissue of the eye, such that functional ABCA4 protein is expressed to compensate for the mutation(s) present in the disease.

The AAV vectors of the disclosure may be formulated as pharmaceutical compositions or medicaments.

An example AAV vector system of the disclosure comprises a first AAV vector and a second AAV vector; wherein the first AAV vector comprises the nucleic acid sequence of SEQ ID NO: 9; and the second AAV vector comprises the nucleic acid sequence of SEQ ID NO: 10.

A further exemplary AAV vector system of the disclosure comprises a first AAV vector and a second AAV vector; wherein the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity; and the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In some embodiments, the methods and uses of the disclosure may also be performed where SEQ ID NO: 2 is used as a reference sequence in place of SEQ ID NO: 1.

In this regard, SEQ ID NO: 2 is identical to SEQ ID NO: 1 with the exception of the following mutations: nucleotide 1640 G>T, nucleotide 5279 G>A, nucleotide 6173 T>C. These mutations do not alter the encoded amino acid sequence, and thus the ABCA4 protein encoded by SEQ ID NO: 2 is identical to the ABCA4 protein encoded by SEQ ID NO: 1.

Thus, in alternative embodiments of the disclosure, references above to SEQ ID NO: 1 may be replaced with references to SEQ ID NO: 2.

In addition, any of the constructs disclosed herein may alternatively comprise a different promoter, such as, e.g., a CMV.CBA promoter, a CBA.RBG promoter, or a CBA.InEx promoter. Similarly, any of the constructs may comprises a 5′ ITR comprising or consisting of SEQ ID NO: 6 and/or a 3′ ITR comprising or consisting of SEQ ID NO: 37.

Sequence Correspondence

As used herein, the term “corresponding to” when used with regard to the nucleotides in a given nucleic acid sequence defines nucleotide positions by reference to a particular SEQ ID NO. However, when such references are made, it will be understood that the disclosure is not to be limited to the exact sequence as set out in the particular SEQ ID NO referred to but includes variant sequences thereof. The nucleotides corresponding to the nucleotide positions in SEQ ID NO: 1 can be readily determined by sequence alignment, such as by using sequence alignment programs, the use of which is well known in the art. In this regard, a skilled person would readily appreciate that the degenerate nature of the genetic code means that variations in a nucleic acid sequence encoding a given polypeptide may be present without changing the amino acid sequence of the encoded polypeptide. Thus, identification of nucleotide locations in other ABCA4 coding sequences is contemplated (i.e. nucleotides at positions which the skilled person would consider correspond to the positions identified in, for example, SEQ ID NO: 1).

By way of example, SEQ ID NO: 2 is identical to SEQ ID NO: 1 with the exception of three specific mutations, as described above (these three mutations do not alter the amino acid sequence of the encoded ABCA4 polypeptide). In this case, a skilled person would therefore consider that a given nucleotide position in SEQ ID NO: 2 corresponded to the equivalent numbered nucleotide position in SEQ ID NO: 1.

### AAV Vectors

The viral vectors of the disclosure comprise adeno-associated viral (AAV) vectors. An AAV vector of the disclosure may be in the form of a mature AAV particle or virion, i.e. nucleic acid surrounded by an AAV protein capsid.

The AAV vector may comprise an AAV genome or a derivative thereof.

An AAV genome is a polynucleotide sequence, which may, in some embodiments, encode functions for the production of an AAV particle. These functions include, for example, those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, an AAV genome of a vector of the disclosure may be replication-deficient.

The AAV genome may be in single-stranded form, either positive or negative-sense, or alternatively in double-stranded form. In some embodiments, the use of a double-stranded form allows bypass of the DNA replication step in the target cell and so can accelerate transgene expression.

In some embodiments, the AAV genome of a vector of the disclosure may be in single-stranded form.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

AAVs are referred to in terms of their serotype. A serotype corresponds to a variant subspecies of AAV which, owing to its profile of expression of capsid surface antigens, has a distinctive reactivity which can be used to distinguish it from other variant subspecies. A virus having a particular AAV serotype does not efficiently cross-react with neutralizing antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the disclosure. Thus, in one embodiment of the disclosure, an AAV vector of the disclosure may be derived from an AAV1, AAV2, AAV3, AAV4, AAV5, AAV6, AAV7, AAV8, AAV9, AAV10, AAV11, Rec2 or Rec3 AAV.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes may be derived from the following accession numbers for AAV whole genome sequences: Adeno-associated virus 1 NC 002077, AF063497; Adeno-associated virus 2 NC 001401; Adeno-associated virus 3 NC 001729; Adeno-associated virus 3B NC 001863; Adeno-associated virus 4 NC 001829; Adeno-associated virus 5 Y18065, AF085716; Adeno-associated virus 6 NC 001862; Avian AAV ATCC VR-865 AY186198, AY629583, NC 004828; Avian AAV strain DA-1 NC_006263, AY629583; Bovine AAV NC_005889, AY388617.

AAV may also be referred to in terms of clades or clones. This refers, for example, to the phylogenetic relationship of naturally derived AAVs, or to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognizably distinct population at a genetic level.

The skilled person can select an appropriate serotype, clade, clone or isolate of AAV for use in the disclosure on the basis of their common general knowledge. For instance, the AAV5 capsid has been shown to transduce primate cone photoreceptors efficiently as evidenced by the successful correction of an inherited color vision defect (Mancuso et al. (2009) Nature 461: 784-7).

The AAV serotype can determine the tissue specificity of infection (or tropism) of an AAV virus. Accordingly, in some preferred embodiments the AAV serotypes for use in AAVs administered to patients of the disclosure are those which have natural tropism for or a high efficiency of infection of target cells within the eye. In one embodiment, AAV serotypes for use in the disclosure are those which infect cells of the neurosensory retina, retinal pigment epithelium and/or choroid.

In some embodiments, the AAV genome of a naturally derived serotype, isolate or clade of AAV comprises at least one inverted terminal repeat sequence (ITR). An ITR sequence may act in cis to provide a functional origin of replication and allows for integration and excision of the vector from the genome of a cell. The AAV genome may also comprise packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins may make up the capsid of an AAV particle. Capsid variants are discussed below.

In some embodiments, a promoter can be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters may be used to express the rep gene, while the p40 promoter may be used to express the cap gene.

In some embodiments, the AAV genome used in a vector of the disclosure may therefore be the full genome of a naturally occurring AAV. For example, a vector comprising a full AAV genome may be used to prepare an AAV vector in vitro. In some embodiments, such a vector may in principle be administered to patients. In some preferred embodiments, the AAV genome will be derivative for the purpose of administration to patients. Such derivatization is known in the art and the disclosure encompasses the use of any known derivative of an AAV genome, and derivatives which could be generated by applying techniques known in the art. Derivatization of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from a vector of the disclosure in vivo. In some embodiments, it is possible to truncate the AAV genome to include minimal viral sequence yet retain the above function. This may contribute to the safety of the AAV genome, by example reducing the risk of recombination of the vector with wild-type virus, and also avoiding triggering a cellular immune response by the presence of viral gene proteins in the target cell.

A derivative of an AAV genome may include at least one inverted terminal repeat sequence (ITR). In some embodiments, a derivative of an AAV genome may include more than one ITR, such as two ITRs or more. One or more of the ITRs may be derived from AAV genomes having different serotypes, or may be a chimeric or mutant ITR. An exemplary mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The inclusion of one or more ITRs may aid concatamer formation of a vector of the disclosure in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In some preferred embodiments, ITR elements will be the only sequences retained from the native AAV genome in the derivative. Thus, a derivative may not include the rep and/or cap genes of the native genome and any other sequences of the native genome. This may also reduce the possibility of integration of the vector into the host cell genome. Additionally, reducing the size of the AAV genome allows for increased flexibility in incorporating other sequence elements (such as regulatory elements) within the vector in addition to the transgene.

The following portions may be removed in a derivative of the disclosure: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. The disclosure encompasses the provision of capsid protein sequences from different serotypes, clades, clones, or isolates of AAV within the same vector (i.e. a pseudotyped vector).

Chimeric, shuffled or capsid-modified derivatives may be selected to provide one or more functionalities for the viral vector. For example, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed, for example, by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins of the disclosure also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. For example, capsid genes may be modified by the insertion of a sequence of an unrelated protein or peptide within an open reading frame of a capsid coding sequence, or at the N- and/or C-terminus of a capsid coding sequence.

The unrelated protein or peptide may be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion may be selected so as not to interfere with other functions of the viral particle e.g. internalization, trafficking of the viral particle. The skilled person can identify suitable sites for insertion based on their common general knowledge. Particular sites are disclosed in Choi et al., referenced above.

The disclosure additionally encompasses the provision of sequences of an AAV genome in a different order and configuration to that of a native AAV genome. The disclosure also encompasses the replacement of one or more AAV sequences or genes with sequences from another virus or with chimeric genes composed of sequences from more than one virus. Such chimeric genes may be composed of sequences from two or more related viral proteins of different viral species.

AAV vectors of the disclosure include transcapsidated forms wherein an AAV genome or derivative having an ITR of one serotype is packaged in the capsid of a different serotype. AAV vectors of the disclosure also include mosaic forms wherein a mixture of unmodified capsid proteins from two or more different serotypes makes up the viral capsid. An AAV vector may also include chemically modified forms bearing ligands adsorbed to the capsid surface. For example, such ligands may include antibodies for targeting a particular cell surface receptor.

Thus, for example, AAV vectors of the disclosure may include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8).

An AAV vector of the disclosure may comprise a mutant AAV capsid protein. In one embodiment, an AAV vector of the disclosure comprises a mutant AAV8 capsid protein. In some embodiments, the mutant AAV8 capsid protein is an AAV8 Y733F capsid protein. In some embodiments, the AAV8 Y733F mutant capsid protein comprises an amino acid sequence with at least 95% identity to SEQ ID NO: 12 with a substitution of phenylalanine for tyrosine at position 733 of SEQ ID NO: 12. In some embodiments, the AAV8 Y733F mutant capsid protein comprises an amino acid sequence of SEQ ID NO: 12 with a substitution of phenylalanine for tyrosine at position 733 of SEQ ID NO: 12.

### AAV RPGR Drug Products

In some embodiments of the compositions of the disclosure, the composition comprises a Drug Product. As used herein, a Drug Product comprises a drug substance, formulated for administration to a subject for the treatment or prevention of a disease or disorder.

The components of an exemplary Drug Product of the disclosure, their functions and specifications are listed in Table 1A.

TABLE 1A
Composition of AAV2-Construct Drug Product

| Name of | | | | |
|---|---|---|---|---|
| Ingredient | Function | Grade | Quantity/Concentration | |
| AAV-Construct | Active | GMP | 2.5 × 10\hat{ }12 | |
| | | | DRP/mL | |
| | | | to 5 × 10\hat{ }12 | |
| | | | DRP/mL | |
| Tris, pH 8.0 | Buffer | EP, BP, USP, | 20 | mM |
| | | JPC | | |
| MgCl₂ | Enhance vector stability | EP, BP, USP, | 1 | mM |
| | | JPC, FCC | | |
| NaCl | Enhance vector stability and | EP, BP, USP, JP | 200 | mM |
| | prevent vector aggregation | | | |

| | | | |
|---|---|---|---|
| Poloxamer 188 | | EP, USP | 0.001% |
| Water for Injections | Diluent | EP, USP | QS to final volume |

### AAV-RPGR Dosage Form

Compositions of the disclosure may be formulated for systemic or local administration.

Compositions of the disclosure may be formulated as a Suspension for Injection or Infusion.

Compositions of the disclosure may be formulated for injection or infusion by any route, including but not limited to, an intravitreous injection or infusion, a subretinal injection or infusion, or a suprachoroidal injection or infusion.

In some embodiments, compositions of the disclosure may be formulated at a concentration of between 0.5×10\hat{ }11 DRP/mL and 1.0×10\hat{ }12 DRP/mL, inclusive of the endpoints. In some embodiments, compositions of the disclosure may be formulated at a concentration of about 0.5×10\hat{ }11 or 0.5×10\hat{ }11 DRP/ml. In some embodiments, compositions of the disclosure may be formulated at a concentration of about 0.5×10\hat{ }11 DRP/mL. In some embodiments, compositions of the disclosure may be formulated at a concentration of about 1×10\hat{ }12 DRP/mL.

Compositions of the disclosure may be diluted prior to administration using a using a diluent of the disclosure. In some embodiments, the diluent is identical to a formulation buffer used for preparation of the AAV-RPGR^{ORF15} Drug Product. In some embodiments, the diluent is not identical to a formulation buffer used for preparation of the AAV-Construct Drug Product.

Compositions of the disclosure, including the AAV-RPGR^{ORF15} construct Drug Product described in Table 1A, may be formulated as a Suspension for Injection containing between 0.5×10\hat{ }11 DRP/mL to 1.0×10\hat{ }13 DRP/mL of AAV particles, inclusive of the endpoints. Compositions of the disclosure, including the AAV-RPGR^{ORF15} construct Drug Product described in Table 1A, may be formulated as a Suspension for Injection containing between 2.5×10\hat{ }12 DRP/mL to 5×10\hat{ }12 DRP/mL DRP/mL of AAV particles. In some embodiments, the AAV-RPGR^{ORF15} Drug Product described in Table 1A, may be formulated as a Suspension for Injection containing 0.5×10\hat{ }11 DRP/mL, 2.5×10\hat{ }12 DRP/mL, 0.5×10\hat{ }12 DRP/mL, 5×10\hat{ }12 DRP/mL or 1.0×10\hat{ }13 DRP/mL of AAV particles. If required by the protocol, AAV-RPGR^{ORF15} Drug Product may be diluted in the clinic (i.e. by a medical professional) before administration using a diluent of the disclosure. In some embodiments, this diluent is the same formulation buffer used for preparation of the AAV-RPGR^{ORF15} Drug Product.

Compositions of the disclosure may comprise full and empty AAV particles. In some embodiments, a full AAV particle comprises a single stranded DNA encoding an AAV-RPGR^{ORF15} construct of the disclosure. The ordinarily skilled artisan can determine whether an AAV particle is full or empty through, for example, transmission electron microscopy analysis, qPCR or ddPCR. In some embodiments of the composition of the disclosure, the composition comprises at least 30%, at least 35%, at least 40%, at least 45%, at least 50%, at least 55%, at least 60%, 65%, at least 67%, at least 69%, at least 70%, at least 71%, at least 72%, at least 73%, at least 76%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% full AAV particles. In some embodiments, the composition comprises at least 70% full AAV particles.

Compositions of the disclosure may be diluted prior to administration using a using a diluent of the disclosure. In some embodiments, the diluent is identical to a formulation buffer used for preparation of the AAV-RPGR^{ORF15} Drug Product. In some embodiments, the diluent is not identical to a formulation buffer used for preparation of the AAV-RPGR^{ORF15} Drug Product.

Compositions of the disclosure, including the AAV-RPGR^{ORF15} Drug Product described in Table 1A, may be formulated as a Suspension for Injection containing between 0.5×10\hat{ }11 DRP/mL and 1.0×10\hat{ }12 DRP/mL, inclusive of the endpoints. In some embodiments, compositions of the disclosure, including the AAV-RPGR^{ORF15} Drug Product described in Table 1A, may be formulated as a Suspension for Injection containing 1.0×10\hat{ }12 DRP/mL to 5×10\hat{ }12 DRP/mL, e.g., 2.5×10\hat{ }12 DRP/mL or 5×10\hat{ }12 DRP/mL. In some embodiments, compositions of the disclosure, including the AAV-RPGR^{ORF15} Drug Product described in Table 1A, may be formulated as a Suspension for Injection containing 0.5×10\hat{ }11 DRP/mL, 2.5×10\hat{ }12 DRP/mL, 5×10\hat{ }12 DRP/mL or 1.0×10\hat{ }12 DRP/mL. If required by the protocol, AAV-RPGR^{ORF15} Drug Product may be diluted in the clinic (i.e. by a medical professional) before administration using a diluent of the disclosure. In some embodiments, this diluent is the same formulation buffer used for preparation of the AAV-RPGR^{ORF15} Drug Product.

### AAV ABCA4 Drug Products

In some embodiments of the compositions of the disclosure, the composition comprises a Drug Product. As used herein, a Drug Product comprises a drug substance, formulated for administration to a subject for the treatment or prevention of a disease or disorder.

The components of an illustrative Drug Product of the disclosure, their functions and specifications are listed in Table 1B.

TABLE 1B
Composition of AAV2-Construct Drug Product

| Name of | | | | |
|---|---|---|---|---|
| Ingredient | Function | Grade | Concentration | |
| AAV-Construct | Active | GMP | 0.5 × 10\hat{ }11 | |
| (Upstream or | | | DRP/mL | |
| Downstream) | | | to 1.0 × 10\hat{ }13 | |
| | | | DRP/mL | |
| Tris, pH 8.0 | Buffer | EP, BP, USP, | 20 | mM |
| | | JPC | | |
| MgCl₂ | Enhance vector stability | EP, BP, USP, | 1 | mM |
| | | JPC, FCC | | |
| NaCl | Enhance vector stability and | EP, BP, USP, JP | 200 | mM |
| | prevent vector aggregation | | | |

| | | | |
|---|---|---|---|
| Poloxamer 188 | | EP, USP | 0.001% |
| Water for Injections | Diluent | EP, USP | QS to final volume |

### AAV-ABCA4 Dosage Form

Compositions of the disclosure may be formulated for systemic or local administration.

Compositions of the disclosure may be formulated as a Suspension for Injection or Infusion.

Compositions of the disclosure may be formulated for injection or infusion by any route, including but not limited to, an intravitreous injection or infusion, a subretinal injection or infusion, or a suprachoroidal injection or infusion.

Compositions of the disclosure may be formulated at a concentration of between 0.5×10\hat{ }11 DRP/mL and 1.0×10\hat{ }12 DRP/mL, inclusive of the endpoints, for an upstream and/or downstream vector, respectively.

Compositions of the disclosure may be diluted prior to administration using a using a diluent of the disclosure. In some embodiments, the diluent is identical to a formulation buffer used for preparation of an AAV-ABCA4 Drug Product. In some embodiments, the diluent is not identical to a formulation buffer used for preparation of the AAV-Construct Drug Product.

Compositions of the disclosure, including an AAV-ABCA4 construct Drug Product described in Table 1B, may be formulated as a Suspension for Injection containing between 0.5×10\hat{ }11 DRP/mL to 1.0×10\hat{ }13 DRP/mL of AAV particles, inclusive of the endpoints, for an upstream and/or downstream vector, respectively. If required by the protocol, AAV-ABCA4 Drug Product may be diluted in the clinic (i.e. by a medical professional) before administration using a diluent of the disclosure. In some embodiments, this diluent is the same formulation buffer used for preparation of the AAV-ABCA4 Drug Product.

Compositions of the disclosure may comprise full and empty AAV particles. In some embodiments, a full AAV particle comprises a single stranded DNA encoding an AAV-ABCA4 construct of the disclosure. The ordinarily skilled artisan can determine whether an AAV particle is full or empty through, for example, transmission electron microscopy analysis, qPCR or ddPCR. In some embodiments of the composition of the disclosure, the composition comprises at least 30%, at least 35%, at least 40%, at least 45%, at least 50%, at least 55%, at least 60%, 65%, at least 67%, at least 69%, at least 70%, at least 71%, at least 72%, at least 73%, at least 76%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% full AAV particles. In some embodiments, the composition comprises at least 70% full AAV particles.

Compositions of the disclosure may be diluted prior to administration using a using a diluent of the disclosure. In some embodiments, the diluent is identical to a formulation buffer used for preparation of the AAV-ABCA4 Drug Product. In some embodiments, the diluent is not identical to a formulation buffer used for preparation of the AAV-ABCA4 Drug Product.

Compositions of the disclosure, including the AAV-ABCA4 Drug Product described in Table 1B, may be formulated as a Suspension for Injection containing between 0.5×10\hat{ }11 DRP/mL and 1.0×10\hat{ }12 DRP/mL, inclusive of the endpoints, for an upstream and/or downstream vector, respectively. If required by the protocol, AAV-ABCA4 Drug Product may be diluted in the clinic (i.e. by a medical professional) before administration using a diluent of the disclosure. In some embodiments, this diluent is the same formulation buffer used for preparation of the AAV-ABCA4 Drug Product.

### AAV-RPGR Pharmaceutical Formulations

Compositions of the disclosure may comprise a Drug Substance. In some embodiments, the Drug Substance comprises or consists of an AAV-RPGR^{ORF15}. In some embodiments, the Drug Substance comprises or consists of an AAV-RPGR^{ORF15} and a formulation buffer. In some embodiments, the formulation buffer comprises 20 mM Tris, 1 mM MgCl₂, and 200 mM NaCl at pH 8. In some embodiments, the formulation buffer comprises 20 mM Tris, 1 mM MgCl₂, and 200 mM NaCl at pH 8 with poloxamer 188 at 0.001%.

### Excipients

Compositions of the disclosure may comprise a Drug Product. In some embodiments, the Drug Product comprises or consists of a Drug Substance and a formulation buffer. In some embodiments, the Drug Product comprises or consists of a Drug Substance diluted in a formulation buffer. In some embodiments, the Drug Product comprises or consists of an AAV8-RPGR^{ORF15} Drug Substance diluted to a final Drug Product AAV-RPGR^{ORF15} vector genome (vg) concentration in a formulation buffer.

### Ocular Formulations

Compositions of the disclosure may be formulated to comprise, consist essentially of or consist of an AAV-RPGR^{ORF15} Drug Substance at an optimal concentration for ocular injection or infusion.

Compositions of the disclosure may comprise one or more buffers that increase or enhance the stability of an AAV of the disclosure. In some embodiments, compositions of the disclosure may comprise one or more buffers that ensure or enhance the stability of an AAV of the disclosure. Alternatively, or in addition, compositions of the disclosure may comprise one or more buffers that prevent, decrease, or minimize AAV particle aggregation. In some embodiments, compositions of the disclosure may comprise one or more buffers that prevent, decrease, or minimize AAV particle aggregation.

Compositions of the disclosure may comprise one or more components that induce or maintain a neutral or slightly basic pH. In some embodiments, compositions of the disclosure comprise one or more components that induce or maintain a neutral or slightly basic pH of between 7 and 9, inclusive of the endpoints. In some embodiments, compositions of the disclosure comprise one or more components that induce or maintain a pH of about 8. In some embodiments, compositions of the disclosure comprise one or more components that induce or maintain a pH of between 7.5 and 8.5. In some embodiments, compositions of the disclosure comprise one or more components that induce or maintain a pH of between 7.7 and 8.3. In some embodiments, compositions of the disclosure comprise one or more components that induce or maintain a pH of between 7.9 and 8.1. In some embodiments, compositions of the disclosure comprise one or more components that induce or maintain a pH of 8.

Following contact of a composition of the disclosure and a cell, the AAV-Construct expresses a gene or a portion thereof, resulting in the production of a product encoded by the gene or a portion thereof. In some embodiments, the cell is a target cell. In some embodiments, the target cell is a retinal cell. In some embodiments, the retinal cell is a neuron. In some embodiments, the neuron is a photoreceptor. In some embodiments, the cell is in vivo, in vitro, ex vivo or in situ. In some embodiments, including those wherein the cell is in vivo, the contacting occurs following administration of the composition to a subject. In some embodiments, the AAV-Construct expresses a gene or a portion thereof, results in the production of a product encoded by the gene or a portion thereof at a therapeutically-effective level of expression of the gene product. In some embodiments, the gene product is a protein.

### Subretinal Batch Formulations

Compositions of the disclosure may be manufactured at a scale of between 1 to 1000 vials per batch, inclusive of the endpoints. In some embodiments of the compositions of the disclosure, a composition, Drug Substance, or Drug Product may be manufactured at a scale of between 50 to 500 vials per batch, inclusive of the endpoints. In some embodiments of the compositions of the disclosure, a composition, Drug Substance, or Drug Product may be manufactured at a scale of between 100 to 250 vials per batch, inclusive of the endpoints.

Exemplary batches of the disclosure may comprise between 0.01 mL and 100 mL, inclusive of the endpoints, of a composition, Drug Substance, or Drug Product of the disclosure.

TABLE 2A
Exemplary Batch Formula for a vial
of AAV-RPGR^{ORF15} Drug Product

| Component | Quantity | | Reference to Standard |
|---|---|---|---|
| AAV-Construct | 5 × 10\hat{ }12 | DRP | In-house, GMP |
| Tris, pH 8.0 | 20 | mM | EP, BP, USP, JPC |
| MgCl₂ (anhydrous) | 1 | mM | EP, BP, USP, JPC, FCC |
| NaCl | 200 | mM | EP, BP, USP, JP |

| | | |
|---|---|---|
| Poloxamer 188 | 0.001% | EP, USP |
| Water For Injections | QS to final volume | EP, USP |

In some embodiments of the methods of the disclosure for preparation of the Drug Product, a Drug Substance is thawed at +35±2° C., and diluted as required in sterile formulation buffer to the target concentration (e.g., 0.5×10\hat{ }12 DRP/mL, 5×10\hat{ }12 DRP/mL or 1.0×10\hat{ }13 DRP/mL).

In some embodiments of the compositions of the disclosure, the target final DRP titre of the AAV-RPGR^{ORF15} Drug Product is 1×10\hat{ }13 DRP/mL, the minimum and maximum acceptable titre is 1.0×10\hat{ }12 DRP/mL and 3.0×10\hat{ }13 DRP/mL, respectively. In some embodiments of the compositions of the disclosure, the target final DRP titre of the AAV-RPGR^{ORF15} Drug Product is 5×10\hat{ }12 DRP/mL. In some embodiments, the AAV-RPGR^{ORF15} Drug Product is sterile filtered and filled into 0.5 ml polypropylene tubes or 0.5 mL Crystal Zenith® (cyclic olefin polymer) vials for either administration following up to a 10× dilution or without dilution.

The vials are then frozen and stored at ≤−60° C. For labelling and storage prior to QP release and distribution to site, the Drug Product is transferred to the qualified clinical distributor. The Drug Product is stored at ≤−60° C. in a temperature monitored freezer until QP release and distribution.

AAV-RPGR^{ORF15} Drug Product may be pre-filled into a microdelivery device for subretinal delivery. Microdelivery devices suitable for subretinal delivery may comprise a microneedle and the AAV-RPGR^{ORF15} Drug Product may be further formulated for prefilled, room temperature or pre-filled cold storage in a microdelivery device.

### Suprachoroidal Batch Formulations

Compositions of the disclosure may be manufactured at a scale of between 1 to 1000 vials per batch, inclusive of the endpoints. In some embodiments of the compositions of the disclosure, a composition, Drug Substance, or Drug Product may be manufactured at a scale of between 50 to 500 vials per batch, inclusive of the endpoints. In some embodiments of the compositions of the disclosure, a composition, Drug Substance, or Drug Product may be manufactured at a scale of between 100 to 250 vials per batch, inclusive of the endpoints.

Exemplary batches of the disclosure may comprise between 0.01 mL and 500 mL, inclusive of the endpoints, of a composition, Drug Substance, or Drug Product of the disclosure.

TABLE 3A
Exemplary Batch Formula for a vial
of AAV-RPGR^{ORF15} Drug Product

| Component | Quantity | | Reference to Standard |
|---|---|---|---|
| AAV-Construct | 5 × 10\hat{ }12 | DRP | In-house, GMP |
| Tris, pH 8.0 | 20 | mM | EP, BP, USP, JPC |
| MgCl₂ (anhydrous) | 1 | mM | EP, BP, USP, JPC, FCC |
| NaCl | 200 | mM | EP, BP, USP, JP |

| | | |
|---|---|---|
| Poloxamer 188 | 0.001% | EP, USP |
| Water For Injections | QS to 125 mL | EP, USP |

In some embodiments of the methods of the disclosure for preparation of the Drug Product, a Drug Substance is thawed at +35±2° C., and diluted as required in sterile formulation buffer to the target concentration (e.g., 0.5×10\hat{ }12 DRP/mL, 5×10\hat{ }12 DRP/mL or 1.0×10\hat{ }13 DRP/mL).

In some embodiments of the compositions of the disclosure, the target final DRP titre of the AAV-RPGR^{ORF15} Drug Product is 1×10\hat{ }13 DRP/mL, the minimum and maximum acceptable titre is 1.0×10\hat{ }12 DRP/mL and 3.0×10\hat{ }13 DRP/mL, respectively. In some embodiments of the compositions of the disclosure, the target final DRP titre of the AAV-RPGR^{ORF15} Drug Product is 5×10\hat{ }12 DRP/mL. In some embodiments, the AAV-RPGR^{ORF15} Drug Product is sterile filtered and filled into 0.5 ml polypropylene tubes or 0.5 mL Crystal Zenith® (cyclic olefin polymer) vials for either administration following up to a 10× dilution or without dilution.

The vials are then frozen and stored at ≤−60° C. For labelling and storage prior to QP release and distribution to site, the Drug Product is transferred to the qualified clinical distributor. The Drug Product is stored at ≤−60° C. in a temperature monitored freezer until QP release and distribution.

AAV-RPGR^{ORF15} Drug Product may be pre-filled into a microdelivery device for suprachoroidal delivery. Microdelivery devices suitable for suprachoroidal delivery may comprise a microcatheter and the AAV-RPGR^{ORF15} Drug Product may be further formulated for prefilled, room temperature or pre-filled cold storage in a microdelivery device.

### AAV-ABCA4 Pharmaceutical Formulations

Compositions of the disclosure may comprise a Drug Substance. In some embodiments, the Drug Substance comprises or consists of an AAV-ABCA4. In some embodiments, the Drug Substance comprises or consists of an AAV-ABCA4 and a formulation buffer. In some embodiments, the formulation buffer comprises 20 mM Tris, 1 mM MgCl₂, and 200 mM NaCl at pH 8. In some embodiments, the formulation buffer comprises 20 mM Tris, 1 mM MgCl₂, and 200 mM NaCl at pH 8 with poloxamer 188 at 0.001%.

### Excipients

Compositions of the disclosure may comprise a Drug Product. In some embodiments, the Drug Product comprises or consists of a Drug Substance and a formulation buffer. In some embodiments, the Drug Product comprises or consists of a Drug Substance diluted in a formulation buffer. In some embodiments, the Drug Product comprises or consists of an AAV8-ABCA4 Drug Substance diluted to a final Drug Product AAV-ABCA4 vector genome (vg) concentration in a formulation buffer.

### Ocular Formulations

Compositions of the disclosure may be formulated to comprise, consist essentially of or consist of an AAV-ABCA4 Drug Substance at an optimal concentration for ocular injection or infusion.

Compositions of the disclosure may comprise one or more buffers that increase or enhance the stability of an AAV of the disclosure. In some embodiments, compositions of the disclosure may comprise one or more buffers that ensure or enhance the stability of an AAV of the disclosure. Alternatively, or in addition, compositions of the disclosure may comprise one or more buffers that prevent, decrease, or minimize AAV particle aggregation. In some embodiments, compositions of the disclosure may comprise one or more buffers that prevent, decrease, or minimize AAV particle aggregation.

Compositions of the disclosure may comprise one or more components that induce or maintain a neutral or slightly basic pH. In some embodiments, compositions of the disclosure comprise one or more components that induce or maintain a neutral or slightly basic pH of between 7 and 9, inclusive of the endpoints. In some embodiments, compositions of the disclosure comprise one or more components that induce or maintain a pH of about 8. In some embodiments, compositions of the disclosure comprise one or more components that induce or maintain a pH of between 7.5 and 8.5. In some embodiments, compositions of the disclosure comprise one or more components that induce or maintain a pH of between 7.7 and 8.3. In some embodiments, compositions of the disclosure comprise one or more components that induce or maintain a pH of between 7.9 and 8.1. In some embodiments, compositions of the disclosure comprise one or more components that induce or maintain a pH of 8.

Following contact of a composition of the disclosure and a cell, the AAV-Construct expresses a gene or a portion thereof, resulting in the production of a product encoded by the gene or a portion thereof. In some embodiments, the cell is a target cell. In some embodiments, the target cell is a retinal cell. In some embodiments, the retinal cell is a neuron. In some embodiments, the neuron is a photoreceptor. In some embodiments, the cell is in vivo, in vitro, ex vivo or in situ. In some embodiments, including those wherein the cell is in vivo, the contacting occurs following administration of the composition to a subject. In some embodiments, the AAV-Construct expresses a gene or a portion thereof, results in the production of a product encoded by the gene or a portion thereof at a therapeutically-effective level of expression of the gene product. In some embodiments, the gene product is a protein.

### Subretinal Batch Formulations

Compositions of the disclosure may be manufactured at a scale of between 1 to 1000 vials per batch, inclusive of the endpoints. In some embodiments of the compositions of the disclosure, a composition, Drug Substance, or Drug Product may be manufactured at a scale of between 50 to 500 vials per batch, inclusive of the endpoints. In some embodiments of the compositions of the disclosure, a composition, Drug Substance, or Drug Product may be manufactured at a scale of between 100 to 250 vials per batch, inclusive of the endpoints.

Exemplary batches of the disclosure may comprise between 0.01 mL and 100 mL, inclusive of the endpoints, of a composition, Drug Substance, or Drug Product of the disclosure.

TABLE 2B
Exemplary Batch Formula for a
vial of AAV-ABCA4 Drug Product

| Component | Quantity | | Reference to Standard |
|---|---|---|---|
| AAV-Construct | | | In-house, GMP |
| Tris, pH 8.0 | 20 | mM | EP, BP, USP, JPC |
| MgCl₂ (anhydrous) | 1 | mM | EP, BP, USP, JPC, FCC |
| NaCl | 200 | mM | EP, BP, USP, JP |

| | | |
|---|---|---|
| Poloxamer 188 | 0.001% | EP, USP |
| Water For Injections | QS | EP, USP |

In some embodiments of the methods of the disclosure for preparation of the Drug Product, a Drug Substance is thawed at +35±2° C., and diluted as required in sterile formulation buffer to the target concentration (e.g., 0.5×10\hat{ }12 DRP/mL, 5×10\hat{ }12 DRP/mL or 1.0×10\hat{ }13 DRP/mL).

In some embodiments of the compositions of the disclosure, the target final DRP titre of the AAV-ABCA4 Drug Product is 1×10\hat{ }13 DRP/mL, the minimum and maximum acceptable titre is 1.0×10\hat{ }12 DRP/mL and 3.0×10\hat{ }13 DRP/mL, respectively. In some embodiments, the AAV-ABCA4 Drug Product is sterile filtered and filled into 0.5 ml polypropylene tubes or 0.5 mL Crystal Zenith® (cyclic olefin polymer) vials for either administration following up to a 10× dilution or without dilution.

The vials are then frozen and stored at ≤−60° C. For labelling and storage prior to QP release and distribution to site, the Drug Product is transferred to the qualified clinical distributor. The Drug Product is stored at ≤−60° C. in a temperature monitored freezer until QP release and distribution.

AAV-ABCA4 Drug Product may be pre-filled into a microdelivery device for subretinal delivery. Microdelivery devices suitable for subretinal delivery may comprise a microneedle and the AAV-ABCA4 Drug Product may be further formulated for prefilled, room temperature or pre-filled cold storage in a microdelivery device.

### Suprachoroidal Batch Formulations

Compositions of the disclosure may be manufactured at a scale of between 1 to 1000 vials per batch, inclusive of the endpoints. In some embodiments of the compositions of the disclosure, a composition, Drug Substance, or Drug Product may be manufactured at a scale of between 50 to 500 vials per batch, inclusive of the endpoints. In some embodiments of the compositions of the disclosure, a composition, Drug Substance, or Drug Product may be manufactured at a scale of between 100 to 250 vials per batch, inclusive of the endpoints.

Exemplary batches of the disclosure may comprise between 0.01 mL and 500 mL, inclusive of the endpoints, of a composition, Drug Substance, or Drug Product of the disclosure.

TABLE 3B
Exemplary Batch Formula for a
vial of AAV-ABCA4 Drug Product

| Component | Quantity | | Reference to Standard |
|---|---|---|---|
| AAV-Construct | | | In-house, GMP |
| Tris, pH 8.0 | 20 | mM | EP, BP, USP, JPC |
| MgCl₂ (anhydrous) | 1 | mM | EP, BP, USP, JPC, FCC |
| NaCl | 200 | mM | EP, BP, USP, JP |

| | | |
|---|---|---|
| Poloxamer 188 | 0.001% | EP, USP |
| Water For Injections | QS to 125 mL | EP, USP |

In some embodiments of the methods of the disclosure for preparation of the Drug Product, a Drug Substance is thawed at +35±2° C., and diluted as required in sterile formulation buffer to the target concentration (e.g., 0.5×10\hat{ }12 DRP/mL, 5×10\hat{ }12 DRP/mL or 1.0×10\hat{ }13 DRP/mL).

In some embodiments of the compositions of the disclosure, the target final DRP titre of the AAV-ABCA4 Drug Product is 1×10\hat{ }13 DRP/mL, the minimum and maximum acceptable titre is 1.0×10\hat{ }12 DRP/mL and 3.0×10\hat{ }13 DRP/mL, respectively. In some embodiments, the AAV-ABCA4 Drug Product is sterile filtered and filled into 0.5 ml polypropylene tubes or 0.5 mL Crystal Zenith® (cyclic olefin polymer) vials for either administration following up to a 10× dilution or without dilution.

The vials are then frozen and stored at ≤−60° C. For labelling and storage prior to QP release and distribution to site, the Drug Product is transferred to the qualified clinical distributor. The Drug Product is stored at ≤−60° C. in a temperature monitored freezer until QP release and distribution.

AAV-ABCA4 Drug Product may be pre-filled into a microdelivery device for suprachoroidal delivery. Microdelivery devices suitable for suprachoroidal delivery may comprise a microcatheter and the AAV-ABCA4 Drug Product may be further formulated for prefilled, room temperature or pre-filled cold storage in a microdelivery device.

### Storage of Compositions

In some embodiments of the compositions of the disclosure, including those wherein the composition comprises a Drug Product and the composition is supplied in a sterile vial, the composition may be stored at below zero (° C.). In some embodiments, the compositions may be thawed and frozen without loss of efficacy of the Drug Product or integrity to the sterile packaging. In some embodiments, the compositions may undergo multiple rounds of thawing and freezing without loss of efficacy of the Drug Product or integrity to the sterile packaging.

In some embodiments of the compositions of the disclosure, including those wherein the composition comprises a Drug Product and the composition is supplied in a sterile vial, the composition may be stored at room temperature.

### Organic Materials

Starting materials used in the preparation of buffers and media of the disclosure are certified as free from material of animal origin.

### Filters and Chromatographic Matrices

Nonlimiting examples of filters used for the filtration of the Drug Substance and Drug Product are Sartopore 0.45 μm and 0.2 μm filters. The filters are non-sterile when purchased and are sterilized in house at the contract manufacturer by autoclaving. In some embodiments, filters are integrity tested by bubble point testing at a threshold pressure (e.g. 3.2 Bars).

All chromatographic materials are released on a Certificate of Analysis prior to use. Columns are purchased prepacked and are sanitized prior to use.

### Methods of Manufacture of AAV-Construct Drug Product

### Cell Build

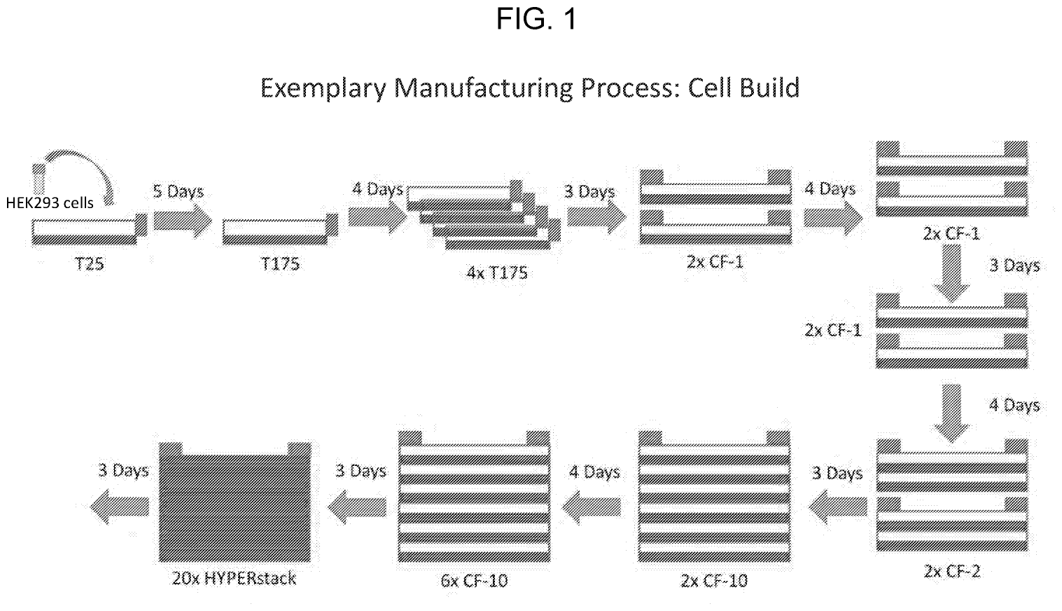

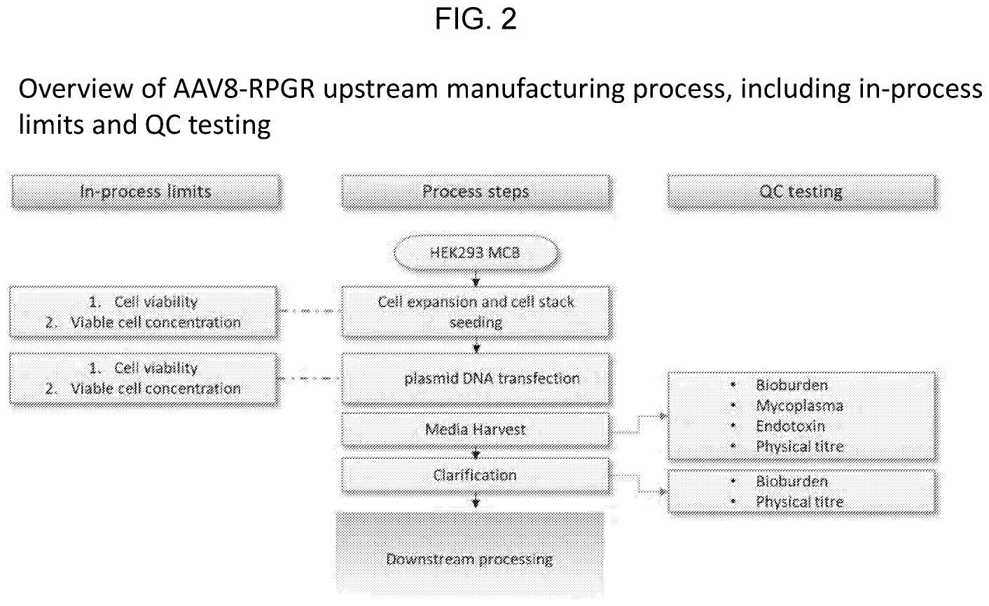

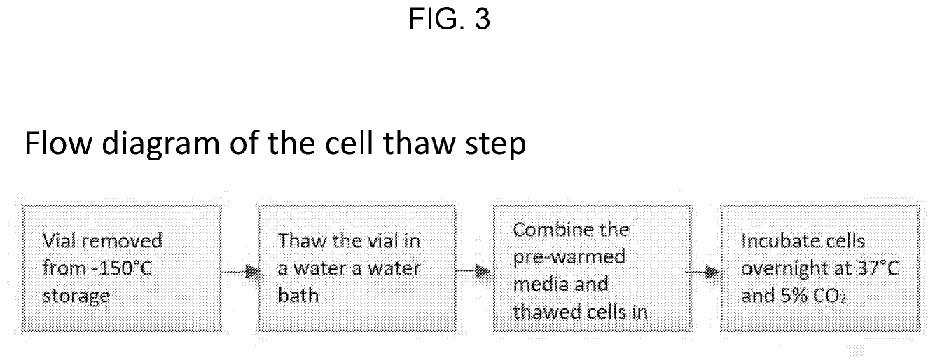

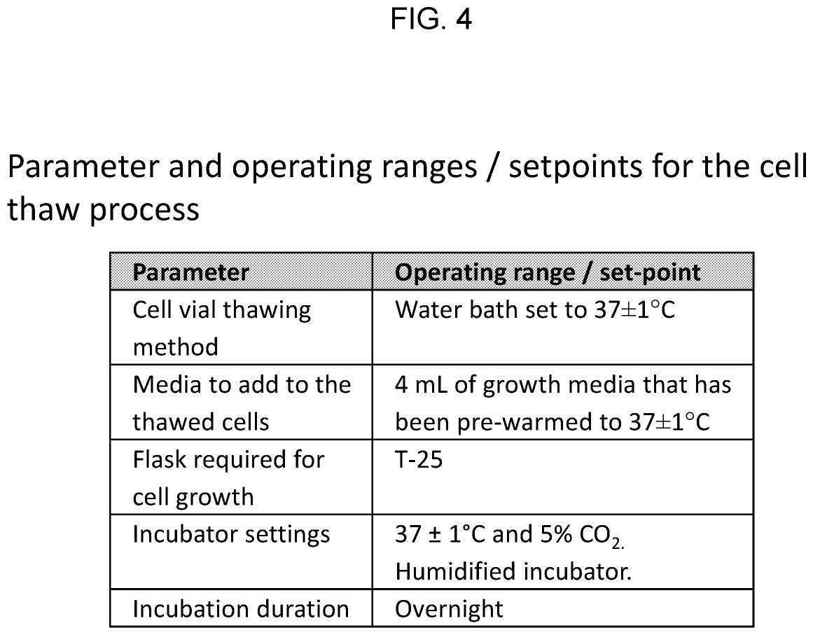

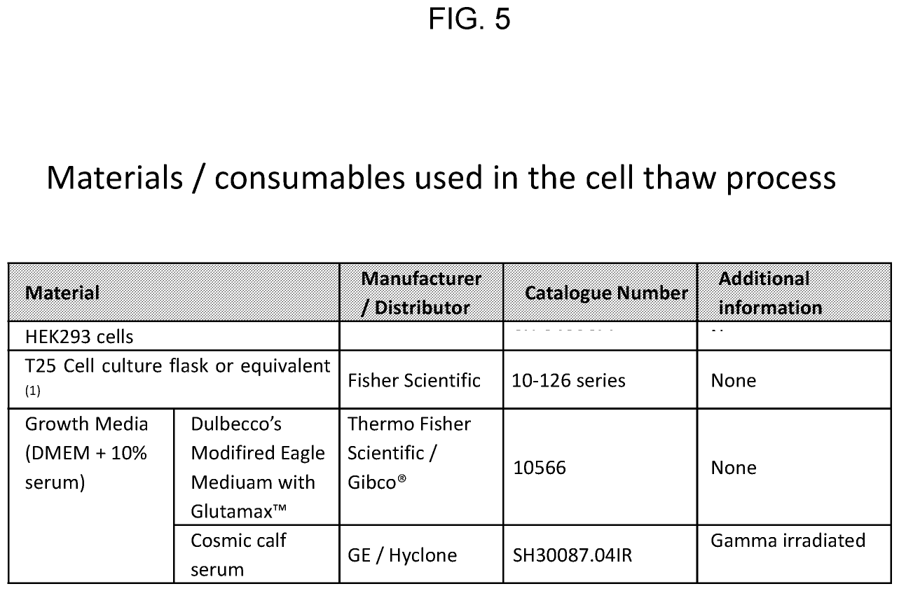

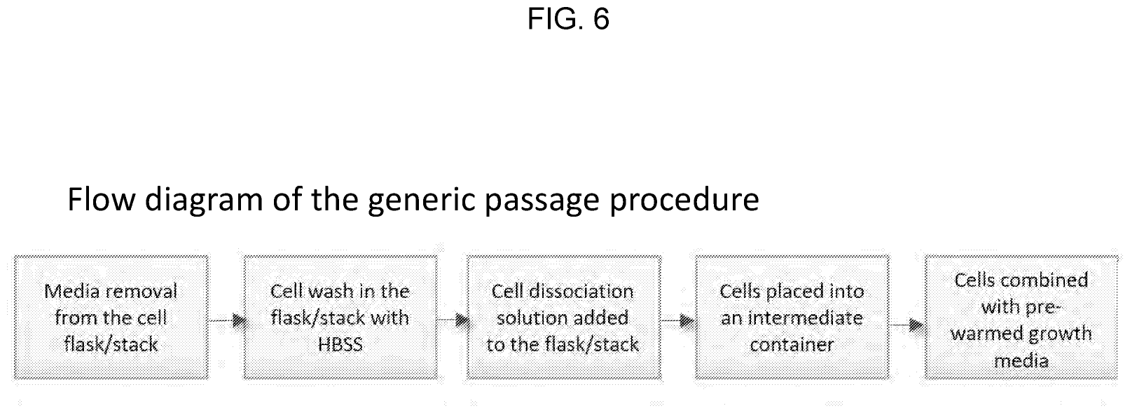

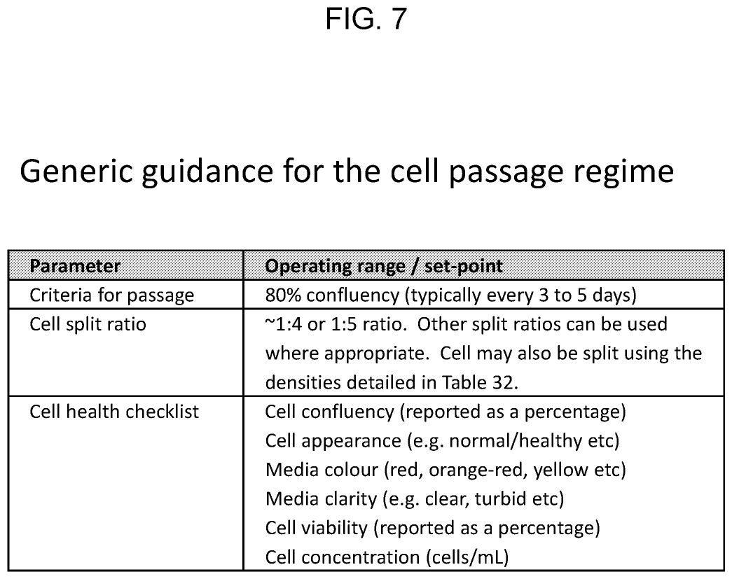

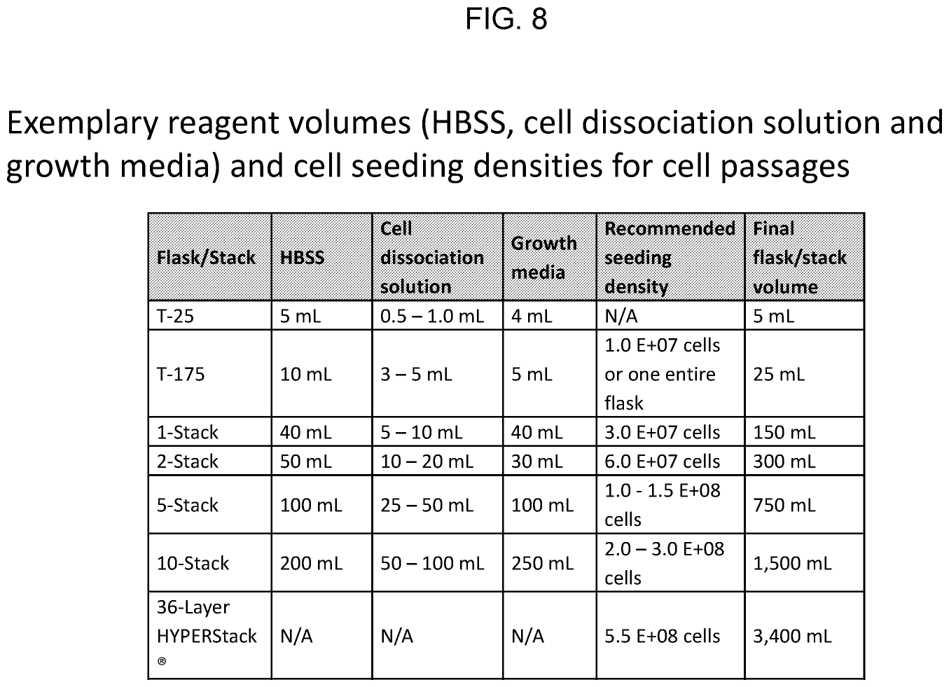

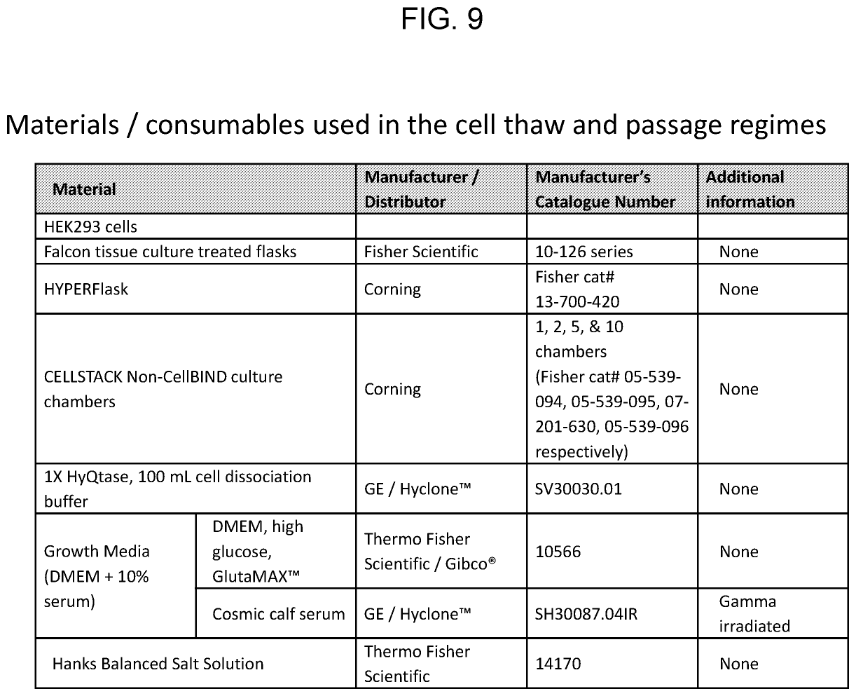

An exemplary passaging protocol comprising 10 passages is shown in FIG. 1. In brief, starting HEK293 cells are cultured for five days in a T25 flask and Growth Medium (Media are summarized in Table 7). After five days HEK293 cells are transferred to a T175 culture flask, cultured for an additional four days, and split into four T175 flasks. Cells are cultured an additional three days, then transferred to two CF-1 Cell Factories (e.g. Nunc™ brand Cell Factories). Cells are cultured an additional four days, transferred to two CF-1 Cell Factories, cultured an additional three days, transferred to two CF-1 Cell Factories, cultured an additional four days, and transferred to two CF-2 Cell Factories. Cells are cultured an additional two days and split into two CF-10 Cell Factories, cultured an additional four days and split into six CF-10 Cell Factories, cultured an additional three days, and transferred to twenty HYPERstacks, which are 36 layered adherent cell culture vessels. Cells are cultured an additional three days in the HYPERstacks prior to transfection.

T25 Flask to T175 Flask: Media is discarded and cells washed with pre-warmed PBS. The cells are loosened with TrypLE cell dissociation reagent. The T-flasks or Cell Stacks are incubated 5 to 10 minutes in an incubator set at 37±1° C. and the cells are fully dislodged by gently tapping the vessel. Growth medium is added to inhibit the TrypLE. The volumes of growth medium, PBS and TrypLE used for different supports are presented in Table 6. All cell suspensions are then pooled.

Cell count and cell viability is determined and the cells are seeded, incubated and passaged in accordance with Table 4.

TABLE 4
Process Parameters for Passages

| | | | Final Flask/ | | |
|---|---|---|---|---|---|
| Passage | | Seeding Density | Stack Volume | | Incubation Time |
| P1 | 1T25 → xT175CB | N/A | 5 | mL | 5 days |
| P2 | 1T175 → 4T175CB | 1.0E+07 cells or one entire | 25 | mL | 4 days |
| | | flask | | | |
| P3 | 4T175CB → 2CF-1 | 1.0E+07 cells or one entire | 150 | mL | 3 days |
| | | flask | | | |
| P4 | 2CF-1→ | 3.0E+07 cells | 150 | mL | 4 days |
| P5 | 2CF-1 → 2CF-1 | 3.0E+07 cells | 150 | mL | 3 days |
| P6 | 2CF-1→ 2CF-2 | 3.0E+07 cells | 150 | mL | 4 days |
| P7 | 2CF-2→ 2CF-10 | 6.0E+07 cells | 300 | mL | 3 days |
| P8 | 2CF-10→ 6CF-10 | 2.0-3.0E+08 cells | 1,500 | mL | 4 days |
| P9 | 6CF-10→ 20x | 2.0-3.0E+08 cells | 1,500 | mL | 3 days |
| | HYPERstack | | | | |
| P10 | 20x HYPERstack→ | 5.5 E+08 cells | 3,400 | mL | 3 days |
| | transfection | | | | |

Cell build processes of the disclosure may be scaled up or down according to the number of HYPERstacks to be used. The use of HYPERstacks provides superior scalability and efficiency of cell culture.

### Transfection

Temperatures, durations, spin speed, and volumes below may be adjusted for optimal results depending upon, among other factors, the cell type used. For the exemplary embodiment described below, conditions were optimized for the use of HEK293 Cells. These methods may be optimized for larger scale production.

An exemplary transfection process used in manufacturing AAV constructs of the disclosure comprises or consists of the steps. Plasmid DNA (e.g., a plasmid encoding an AAV Construct comprising an RPGR^{ORF15} or an ABCA4 sequence, a plasmid encoding Ad5 helper functions and a plasmid encoding AAV8 rep and cap genes) and a transfection composition (for example, comprising a polymer or PEI) are diluted separately into transfection solution to produce a DNA transfection composition and a diluted transfection composition, respectively. The diluted transfection composition is added to the DNA transfection composition and incubated at room temperature to produce a Transfectable DNA Composition. The resulting Transfectable DNA Composition is added to the Transfection Medium. Growth Medium is removed from the HYPERstack, and Transfection Medium comprising the Transfectable DNA Composition is added to the empty HYPERstack. The HYPERstack is incubated at 37° C., 5% CO2 for at least 12, 16, 20, 24, 28, 32, 36, 40, 44, or 48 hours.

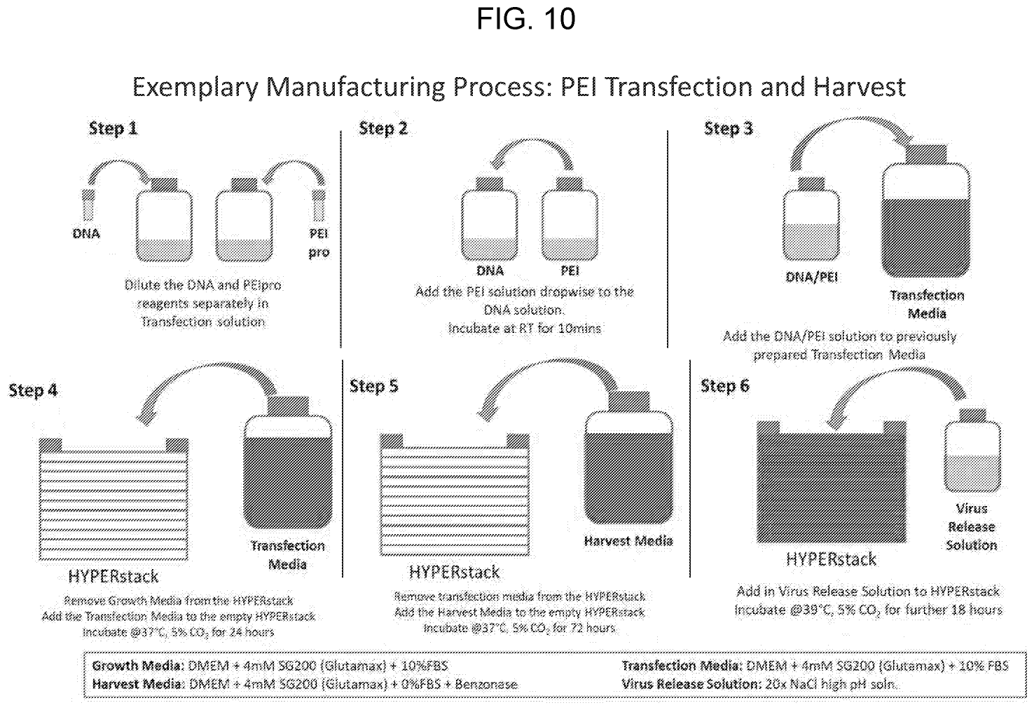

A summary of an exemplary transfection process used in manufacturing AAV constructs of the disclosure is shown in FIG. 10. Plasmid DNA (e.g., a plasmid encoding an AAV Construct comprising an RPGR^{ORF15} or an ABCA4 sequence, and a plasmid encoding AAV8 rep and cap genes) and a polyethylenimine (PEI) transfection reagent, PEIpro® (Polyplus Transfection) are diluted separately into transfection solution. The PEIpro® solution is added dropwise to the DNA solution and incubated for 10 minutes at room temperature. The resulting DNA/PEIpro® solution is added to the Transfection Medium. Growth Medium is removed from the HYPERstack, and Transfection Medium comprising the DNA/PEIpro® solution is added to the empty HYPERstack. The HYPERstack is incubated at 37° C., 5% CO2 for 24 hours.

TABLE 5
Transduction Conditions
Transduction Conditions

| | |
|---|---|
| Working Volume | 3900 mL/HYPERstack |
| DNA + Transfection Media | mL/HYPERstack |
| Total DNA Quantity | XX mg/HYPERstack |
| Plasmid DNA | pAAV.RPGR^{ORF15} |
| | pHELP- |
| | pNLRep-Cap8 |
| PEIpro + Transfection Media | XX mL/HYPERstack |
| PEIpro:DNA Ratio | (1:1 to 3:1) |

The plasmids and the PEI are prepared with Transfection Medium (Table 5).

In certain embodiments, the amount of plasmid DNA is presented in Table 6.

TABLE 6
Plasmid DNA Amounts

| | | Quantity (mg) for |
|---|---|---|
| Plasmid | Ratio | 20x HYPERstack |
| pAAV.RPGR^{ORF15} orpAAV- | 1 | 6 |
| ABCA4 | | |
| pHELP | 2 | 12 |
| pNLRep-Cap | 1.5 | 9 |

In particular embodiments, the PEIpro® to DNA ratio (mL:mg) is about 0.5:1 to about 5:1, or about 1:1 to about 5:1, respectively, optionally about 2:1 to about 4:1, about 4:1, about 3:1, or about 2:1. In certain embodiments, the transfection is conducted using PEI, wherein the plasmid vector comprising an exogenous sequence, the helper plasmid vector, and the plasmid vector comprising a sequence encoding a viral Rep protein and a viral Cap protein are provided in a molar ratio of 1:1:1, respectively. In certain embodiments, the transfection is conducted using PEI at a PEI:DNA ratio (mL:mg) of about 0.5:1 to 5:1 or about 1:1 to about 5:1, respectively, optionally about 2:1 to about 4:1, about 4:1, about 3:1, or about 2:1, wherein the plasmid vector comprising an exogenous sequence, the helper plasmid vector, and the plasmid vector comprising a sequence encoding a viral Rep protein and a viral Cap protein are provided in a molar ratio of about 0.5:1:1 to about 10:1:1, about 1:1:1 to about 10:1:1, about 2:1:1 to about 10:1:1 optionally about 0.5:1:1, about 1:1:1, about 2:1:1, about 3:1:1, about 4:1:1, about 5:1:1, about 6:1:1, about 7:1:1, about 8:1:1, about 9:1:1, or about 10:1:1. In certain embodiments, the transfection is conducted using PEI (e.g., PEIpro®) at a PEI:DNA (mL:mg) ratio of about 1:1 to about 5:1, respectively, optionally about 2:1 to about 4:1, about 4:1, about 3:1, or about 2:1, wherein the plasmid vector comprising an exogenous sequence, the helper plasmid vector, and the plasmid vector comprising a sequence encoding a viral Rep protein and a viral Cap protein are provided in a molar ratio of 1:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of 3:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of 10:1:1, respectively. In some embodiments, the plasmid vector comprising an exogenous sequence, the helper plasmid vector, and the plasmid vector comprising a sequence encoding a viral Rep protein and a viral Cap protein are provided in a molar ratio of about 2:1:1, about 3:1:1, about 4:1:1, about 5:1:1, about 6:1:1, about 7:1:1, about 8:1:1, or about 9:1:1, respectively.

Following transfection, Transfection Medium is removed from the HYPERstack, and Harvest Medium is added. Cells are incubated in Harvest Medium at 37° C., 5% CO2 for 72 hours. Virus Release Solution is added to the HYPERstack at a ratio of 1:20 by volume, and cells are incubated at 37° C., 5% CO2 for 18 hours.

Exemplary formulations of the different types of Media and solutions used to culture cells, transfect cells, and release AAV viral particles are disclosed in Table 7.

TABLE 7
Media and Solutions

| | |
|---|---|
| Growth Media | Dulbecco's Modified Eagle Medium |
| | (DMEM), 4 mM stabilized glutamine or |
| | stabilized glutamine dipeptide, 10% Fetal |
| | Bovine Serum (FBS) |
| Transfection Media | DMEM, 4 mM stabilized glutamine or |
| | stabilized glutamine dipeptide, 10% FBS |
| Harvest Media | DMEM, 4 mM stabilized glutamine or |
| | stabilized glutamine dipeptide, 0% FBS, |
| | Benzonase |
| Virus Release Solution | 20x NaCl high pH solution |

### Harvest

Following incubation with Virus Release Solution, Harvest Media containing AAV viral particles released from the transfected HEK293 cells is removed from the HYPERstack. In some embodiments, the AAV viral particles are purified from the Harvest Media.

### Down Stream Processing

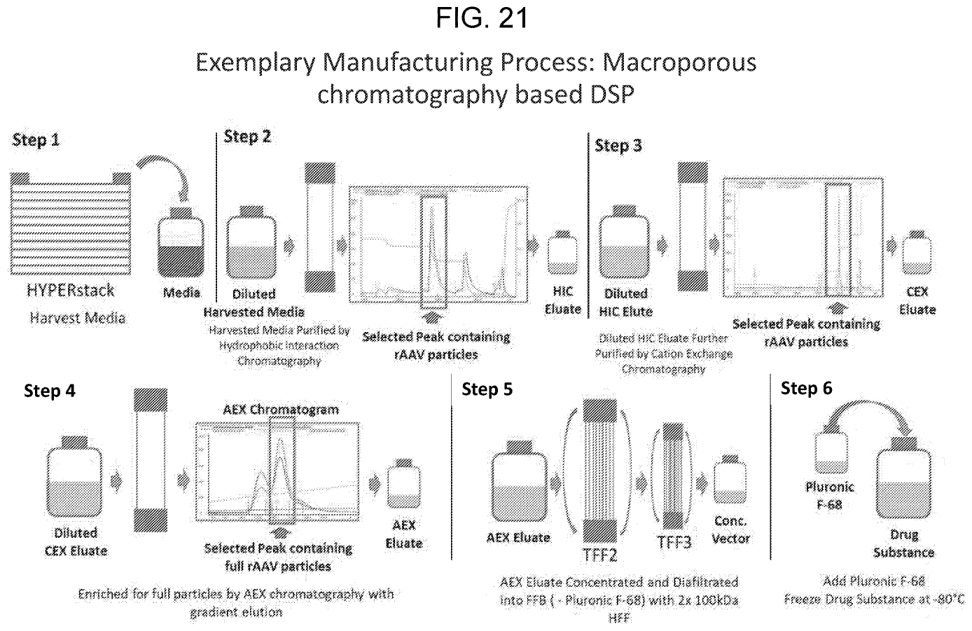

A summary of exemplary down stream processing steps is shown in FIG. 21 and described in the accompanying Examples. In brief, after collecting the Harvest Media comprising the plurality of AAV particles, the plurality of AAV particles are purified though hydrophobic interaction chromatography (HIC) to produce a HIC eluate comprising the plurality of AAV particles. The HIC eluate is diluted, and the plurality of AAV particles are further purified through cation exchange chromatography (CEX) to produce a CEX eluate comprising a plurality of rAAV particles. The plurality of rAAV particles from the CEX are purified by anion exchange (AEX) chromatography to enrich for full rAAV particles. Finally, the AEX eluate comprising a plurality of purified and enriched rAAV particles is diafiltered and concentrated into a formulation buffer by tangential flow filtration (TFF) to produce a final composition comprising a purified and enriched plurality of full rAAV particles and the formulation buffer. In some embodiments, poloxamer 188 is added to the formulation buffer and the Drug Substance is frozen at <−60° C. In some embodiments, the TFF step comprises a single TFF procedure. In some embodiments, the TFF step comprises two or more sequential TFF procedures.

### Hydrophobic Interaction Chromatography

Hydrophobic Interaction Chromatography (HIC) captures rAAV viral particles based on the binding of viral capsid proteins to the matrix of the chromatography column through hydrophobic interactions. In some embodiments, a high salt concentration is used to promote clustering of hydrophobic surfaces.

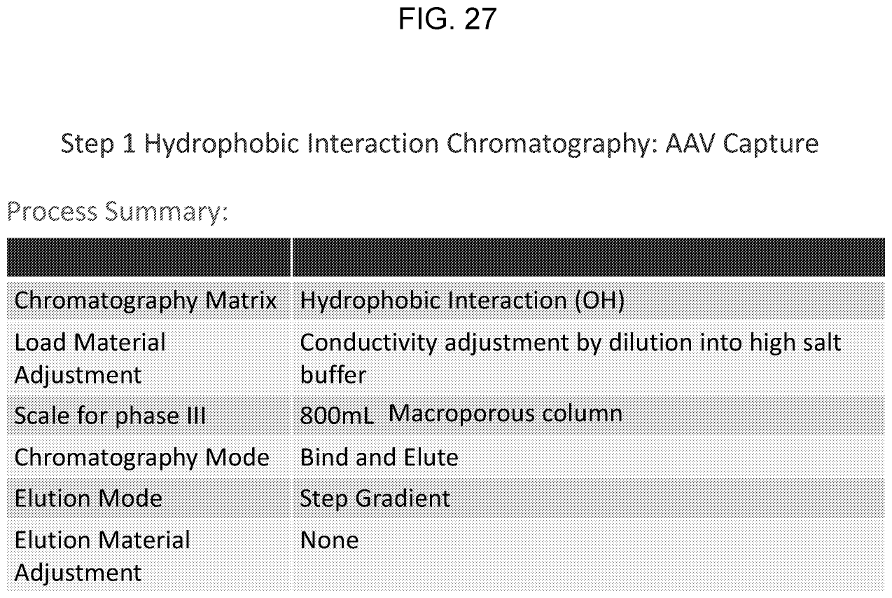

A process summary of an exemplary Hydrophobic Interaction Chromatography (HIC) step of the manufacturing processes of the disclosure is shown in FIG. 27.

In some embodiments, the HIC step comprises the steps of: (i) diluting the harvest media comprising a plurality of released rAAV particles; (ii) loading the diluted harvest media on a HIC column; (iii) generating a HIC chromatogram; and (iv) selecting a peak on the HIC chromatogram containing rAAV particles to produce the HIC eluate comprising a plurality of rAAV viral particles. In some embodiments, the chromatography matrix used in the HIC column is a Hydrophobic Interaction (OH) matrix. In some embodiments, the HIC column is an 800 mL monolith. In some embodiments, the harvest media is diluted into a high salt buffer. In some embodiments, a step gradient to elute the rAAV particles. In some embodiments, an isocratic elution to elute the rAAV particles. Illustrative buffer conditions are provided, e.g., in FIGS. 212 and 336.

In some embodiments, the HIC eluate is diluted and filtered prior to additional purification, i.e. prior to Cation Exchange Chromatography (CEX). A suitable filter is chosen to minimize loss of rAAV particles during filtration. In some embodiments, the HIC eluate is filtered using a 0.8/0.45 μM polyethersulfone (PES) filter.

### Cation Exchange Chromatography

In some embodiments of the methods of the disclosure, Cation Exchange Chromatography (CEX) can be used to further purify the plurality of rAAV particles in the HIC eluate produced by the HIC step. CEX is a type of ion exchange chromatography, which separates molecules based on their net surface charge. In some embodiments, CEX uses a negatively charged ion exchange resin with an affinity for positively charged molecules. When the pH is below the isoelectric point (pI) of the AAV particles, the AAV viral particles have a positive charge and can be purified by CEX.

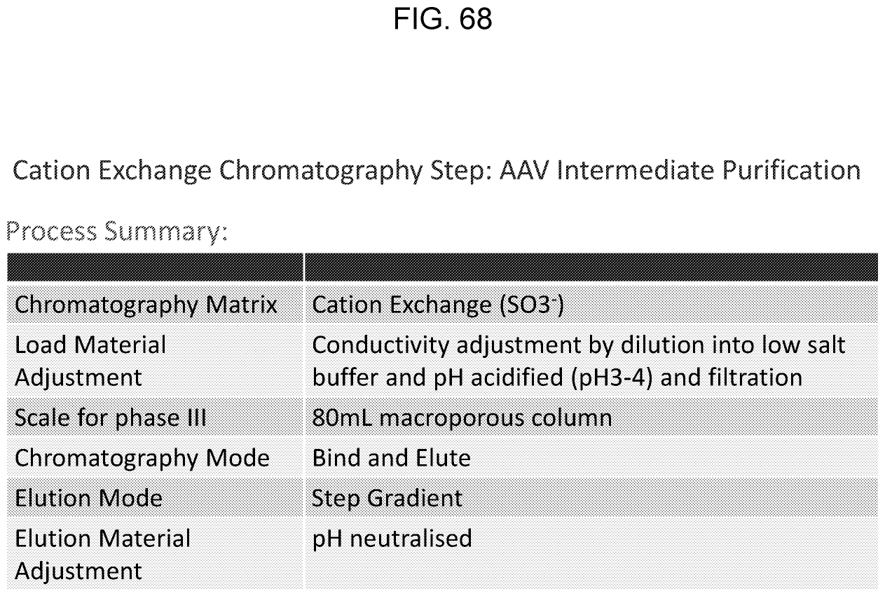

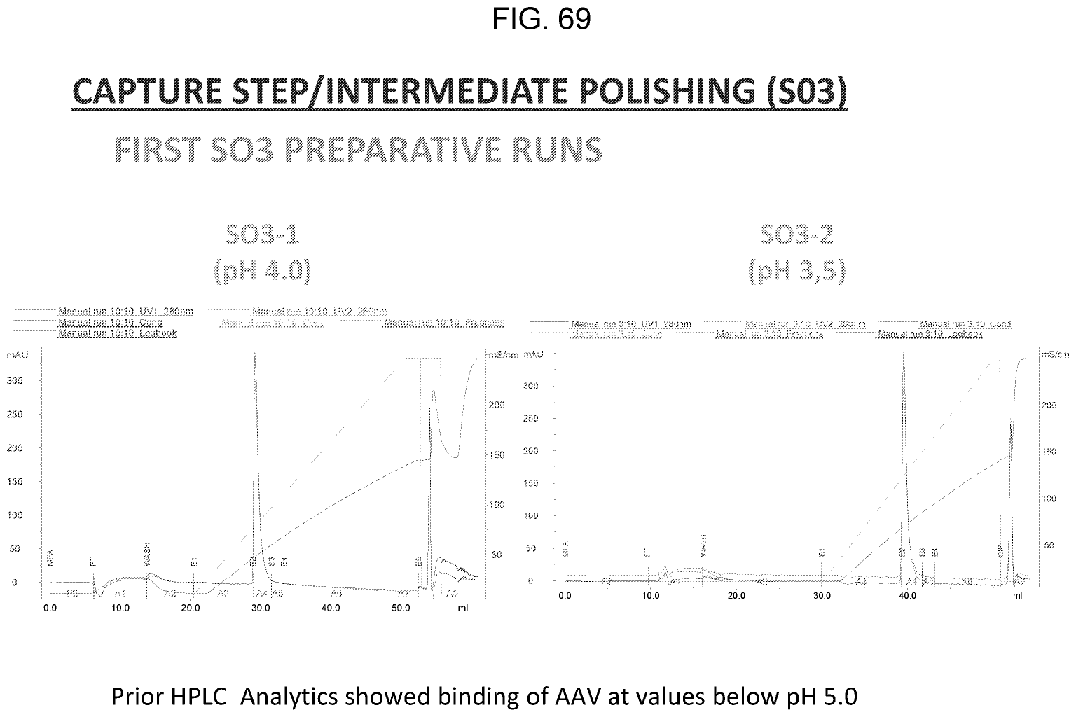

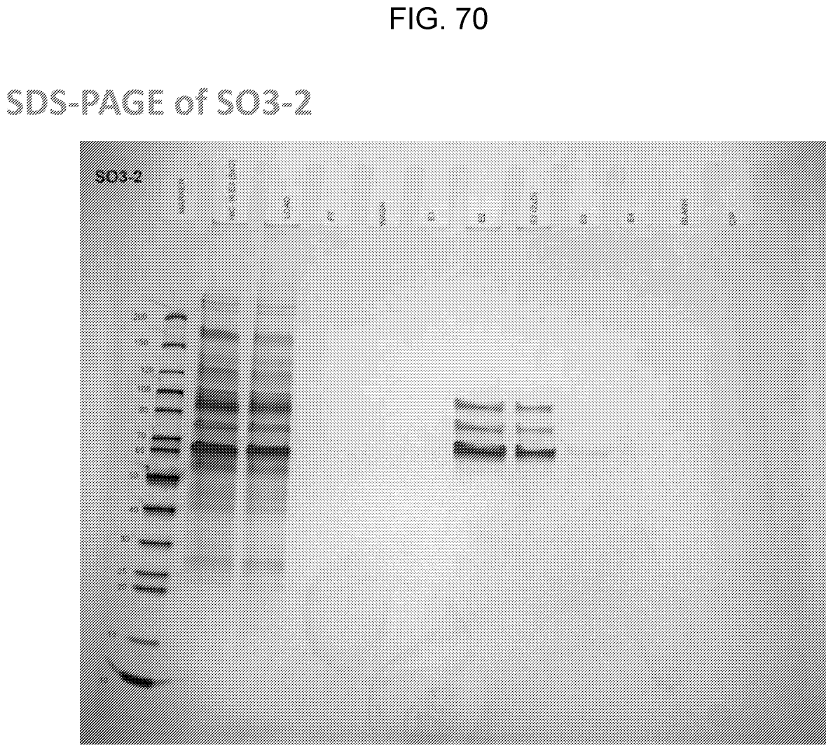

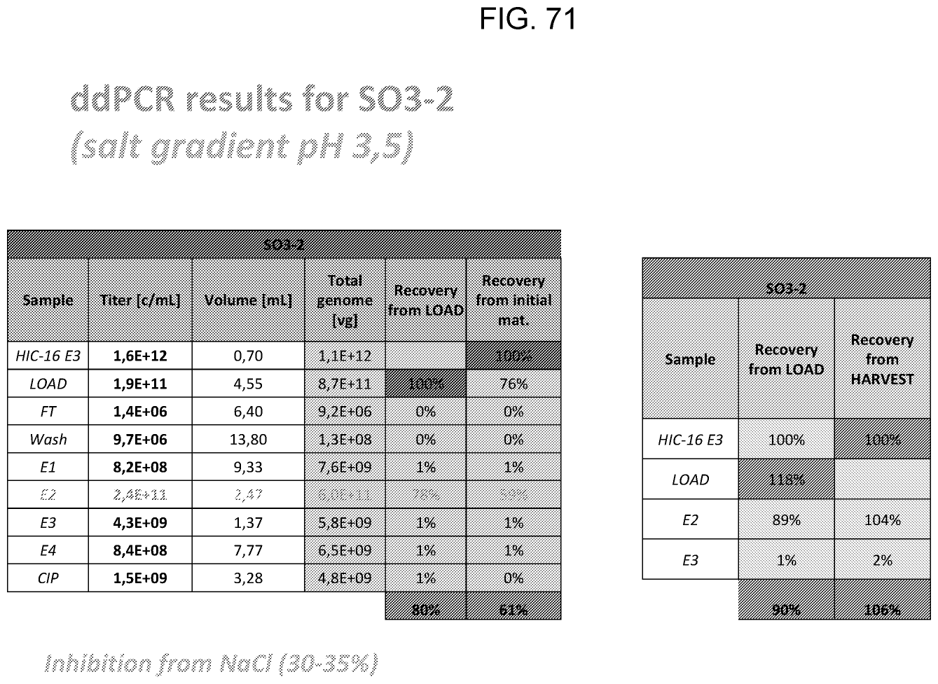

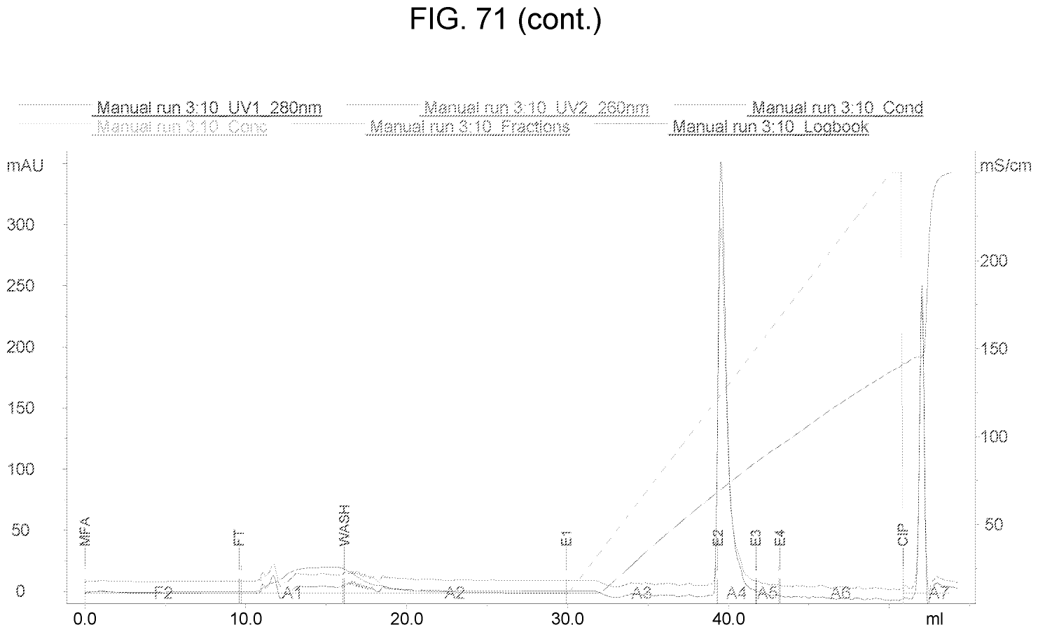

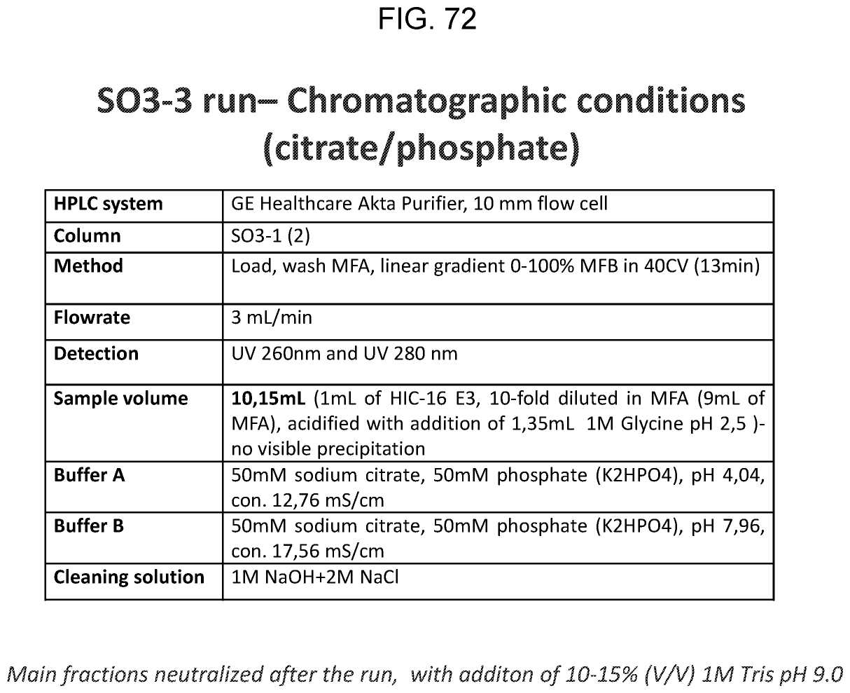

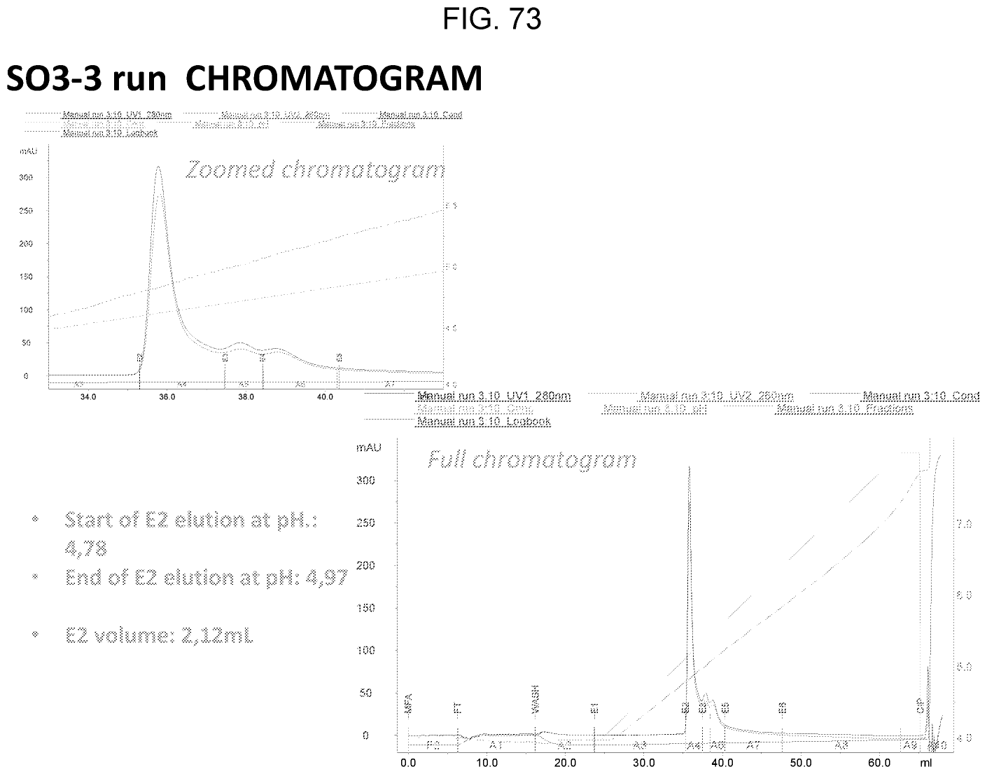

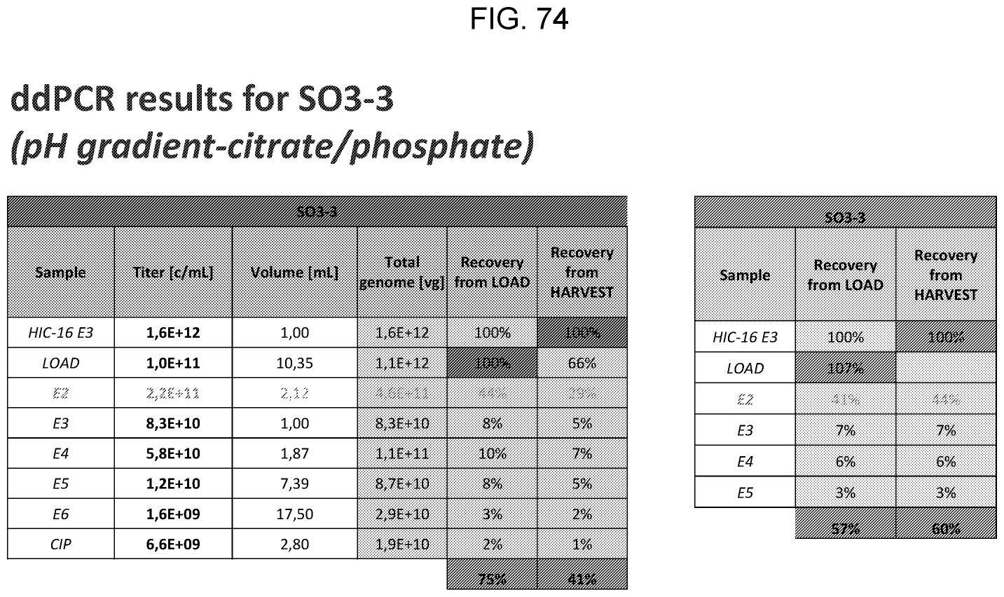

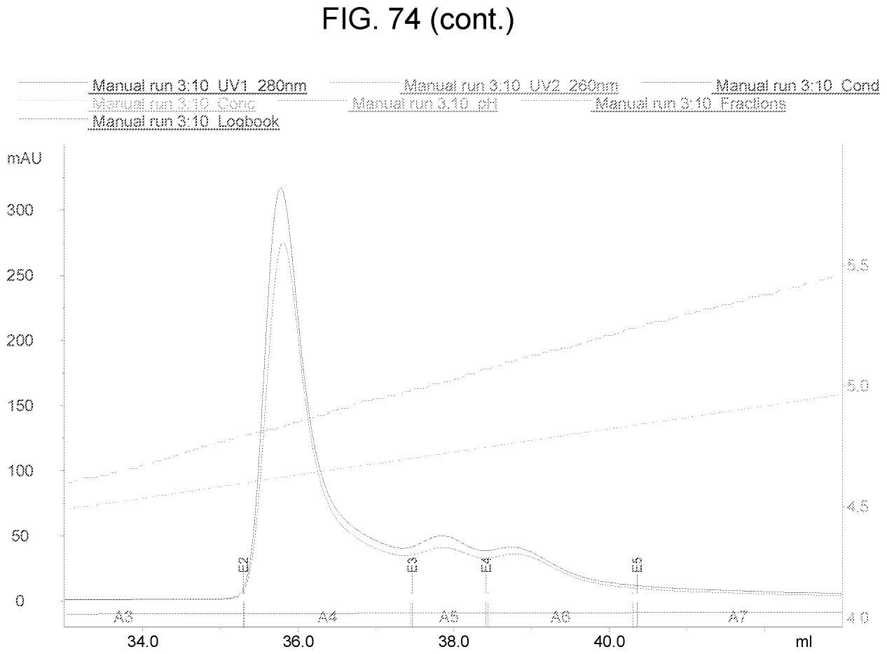

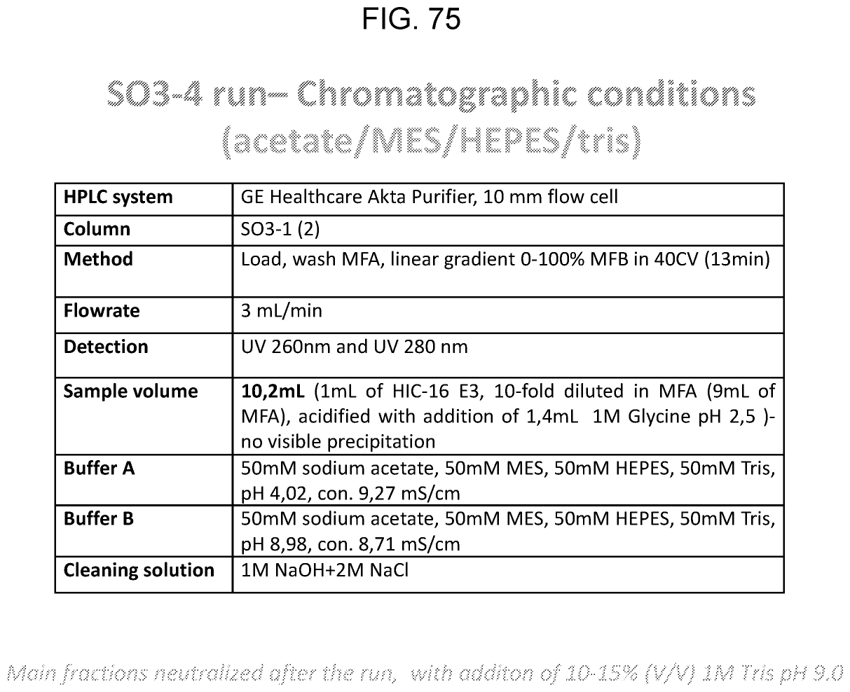

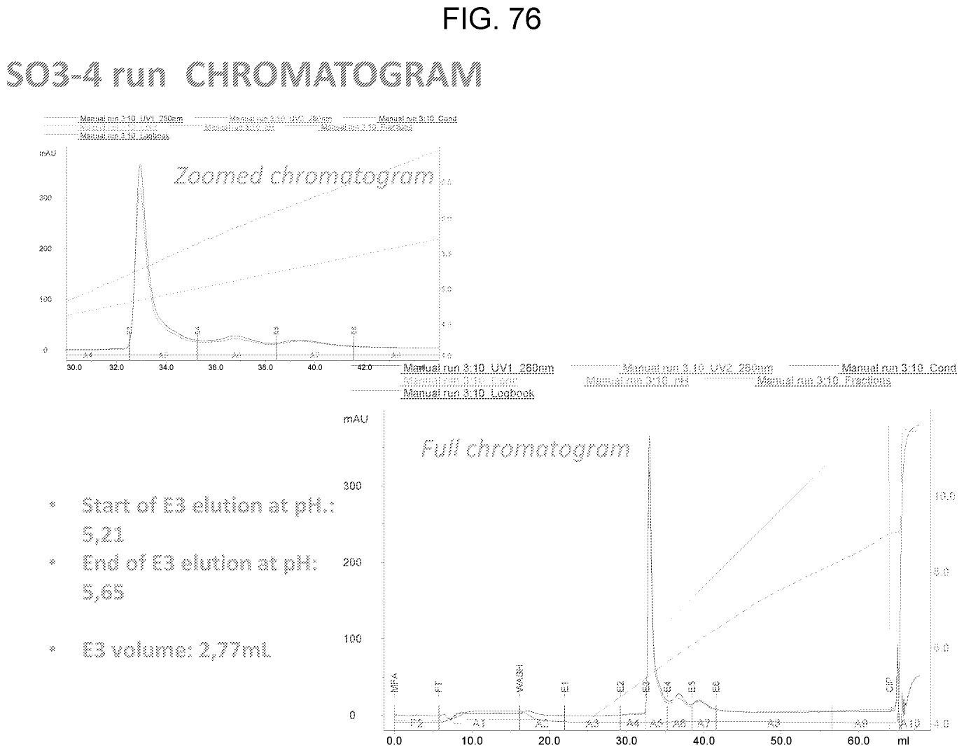

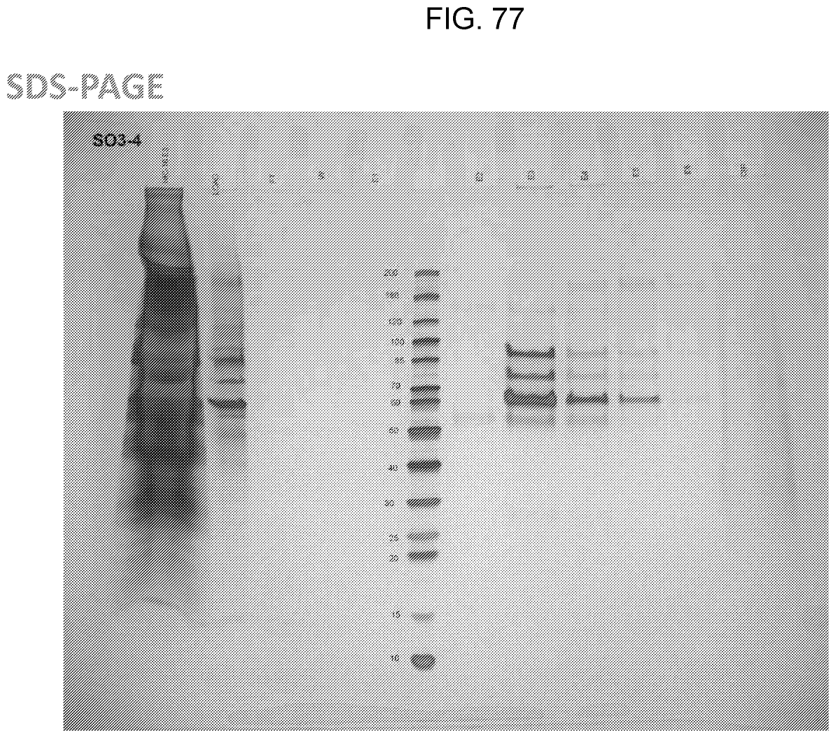

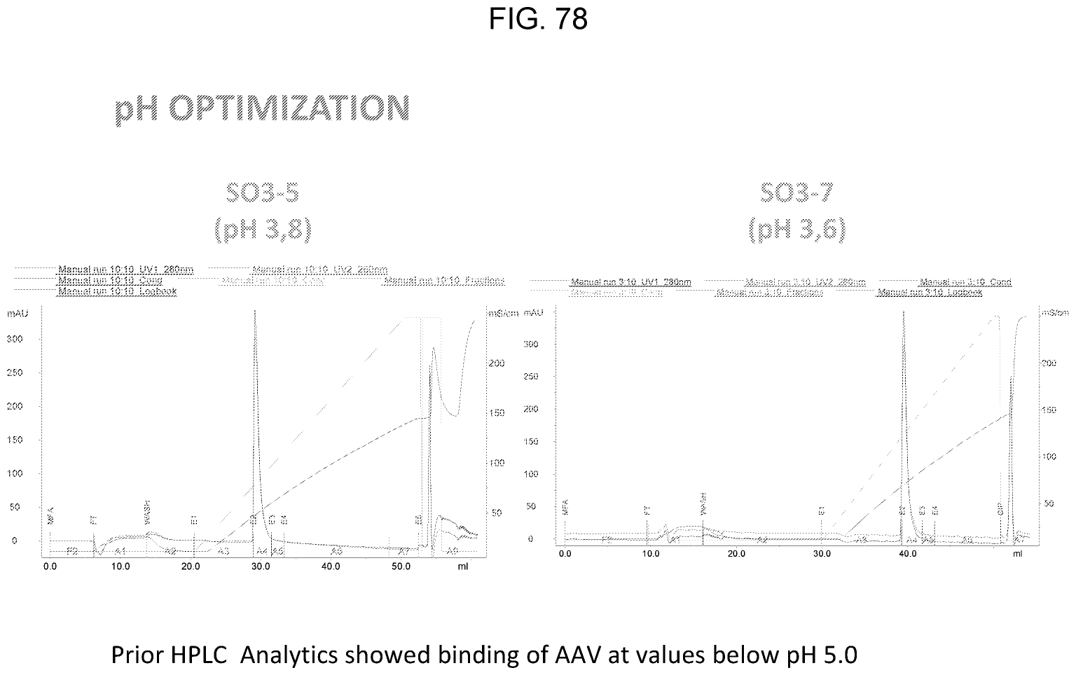

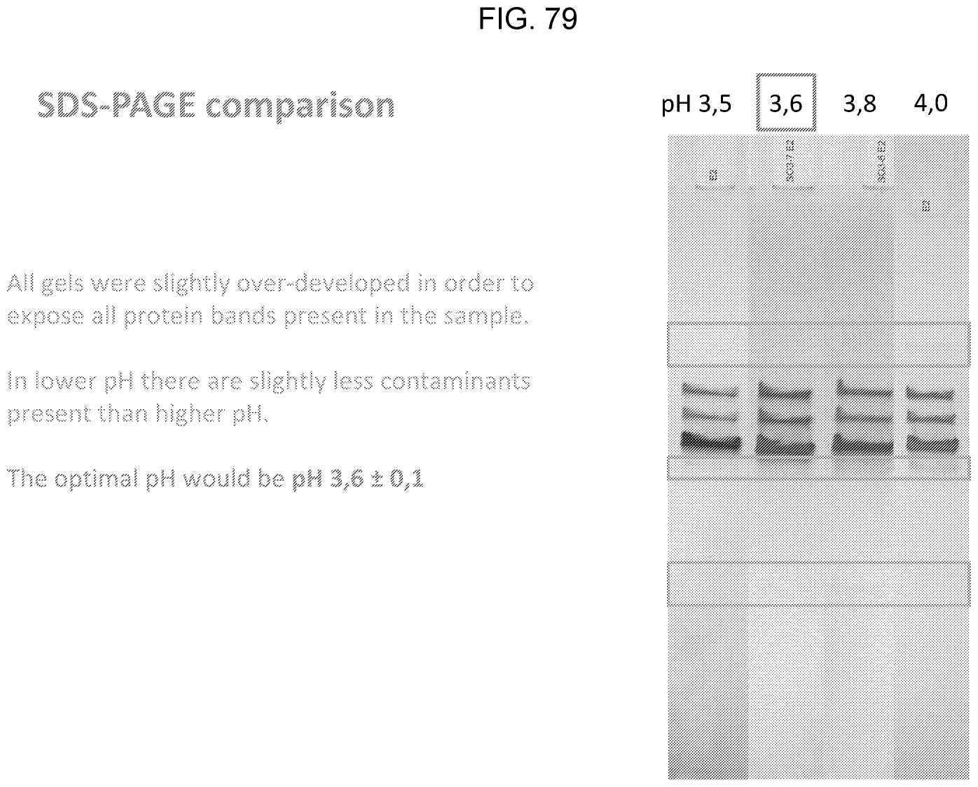

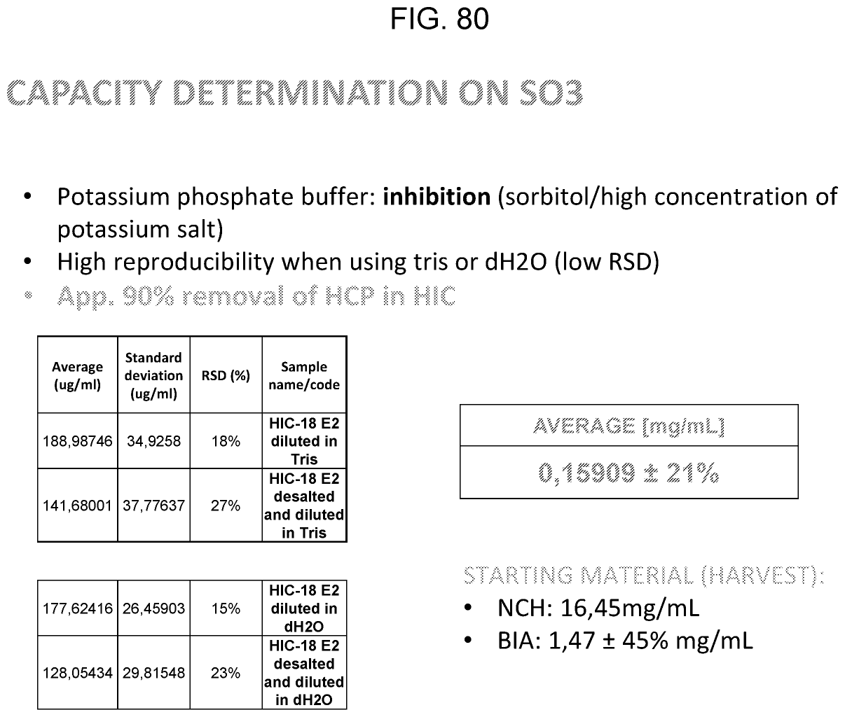

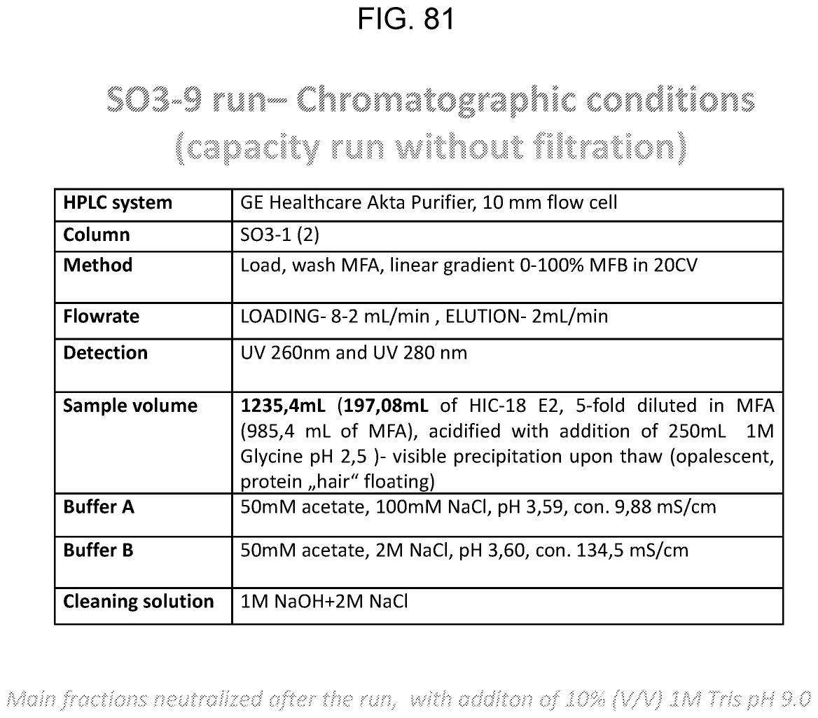

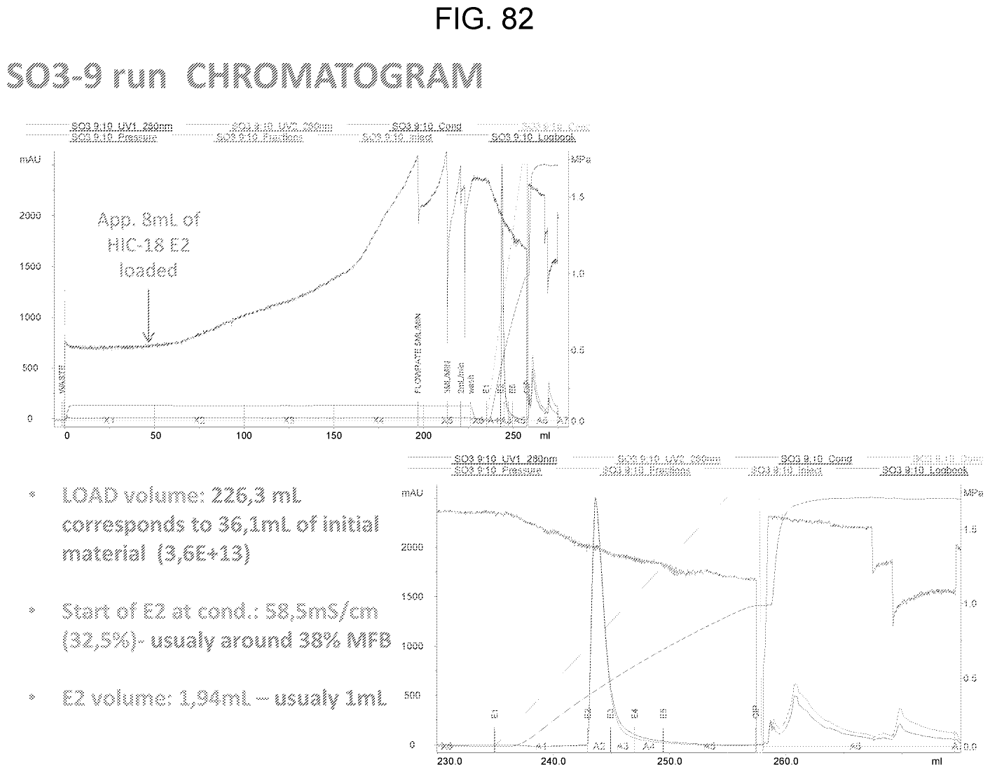

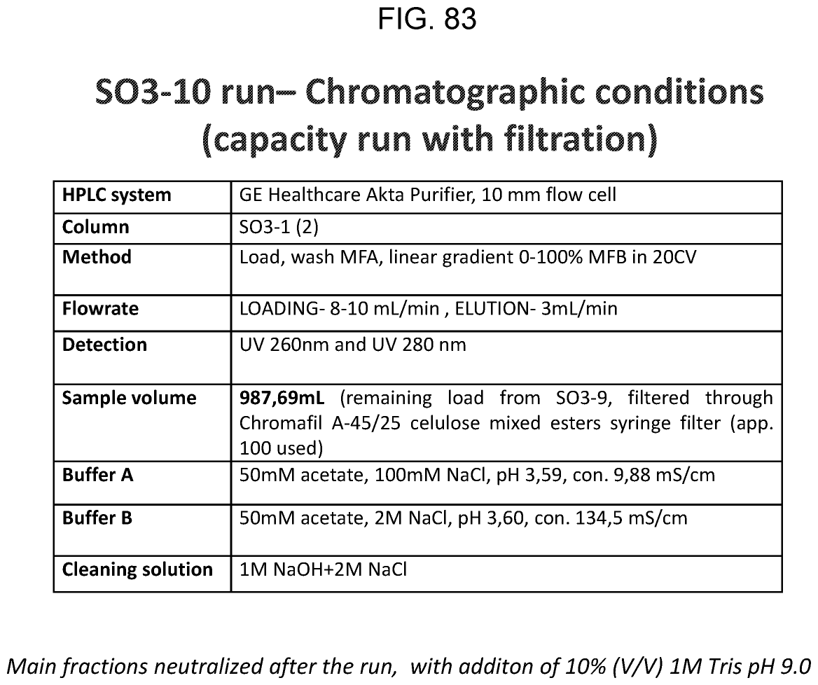

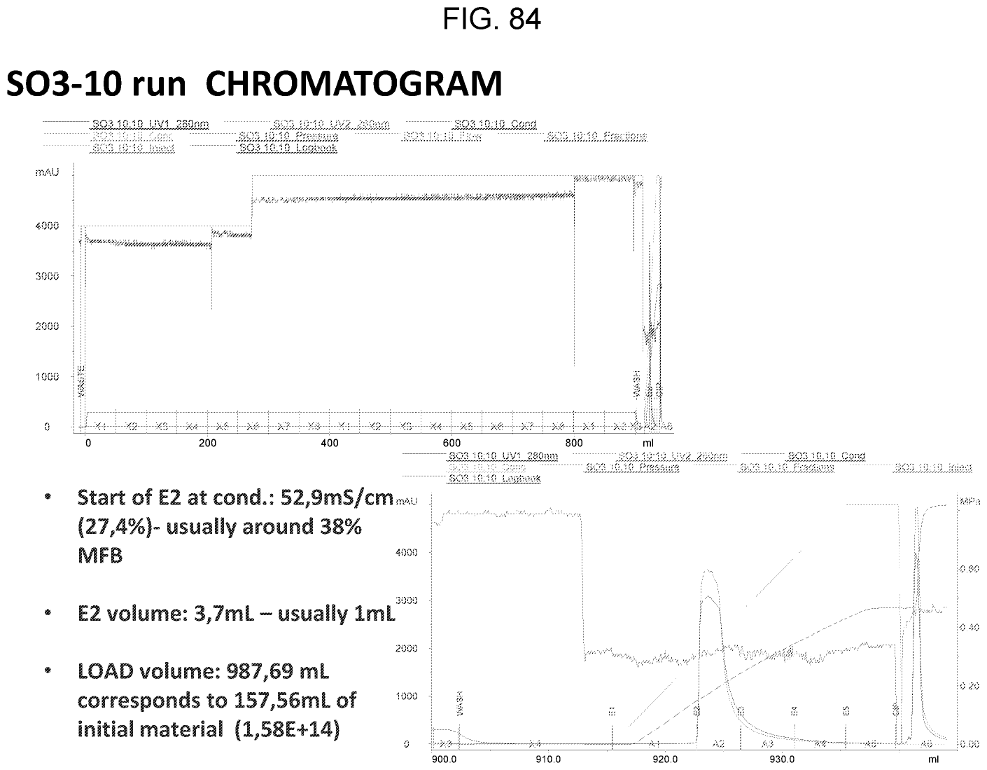

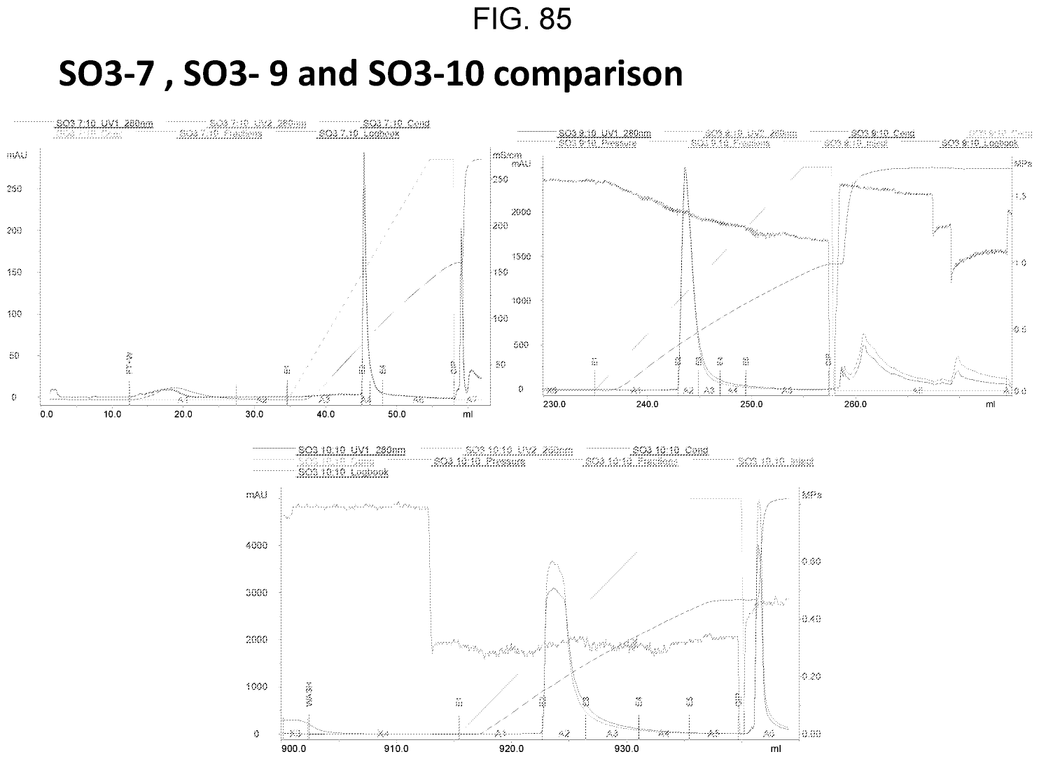

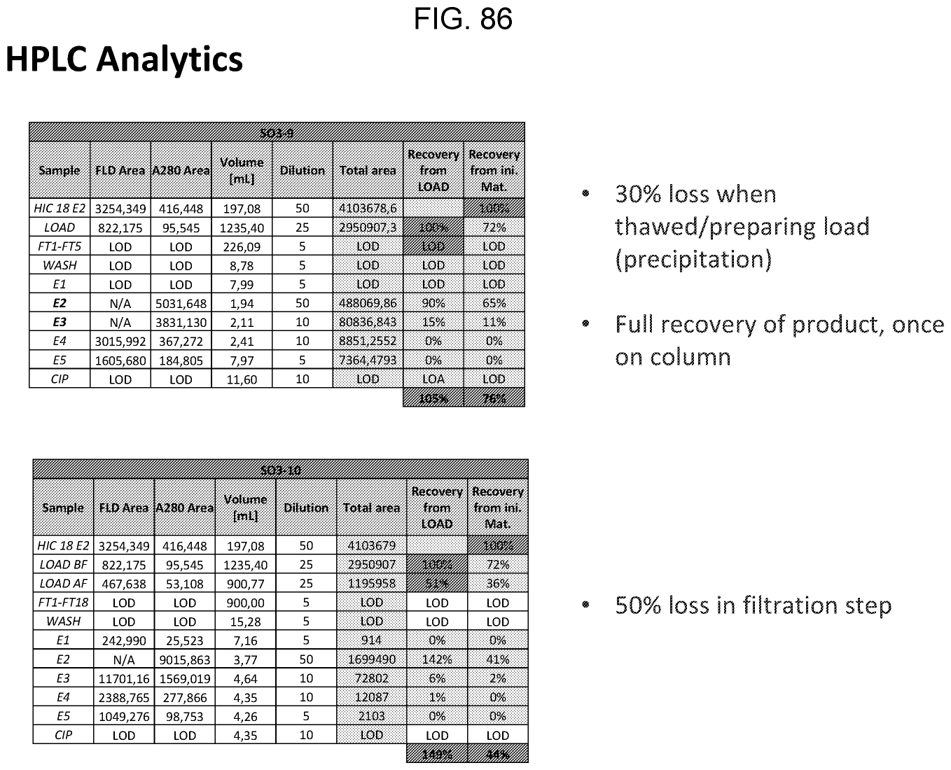

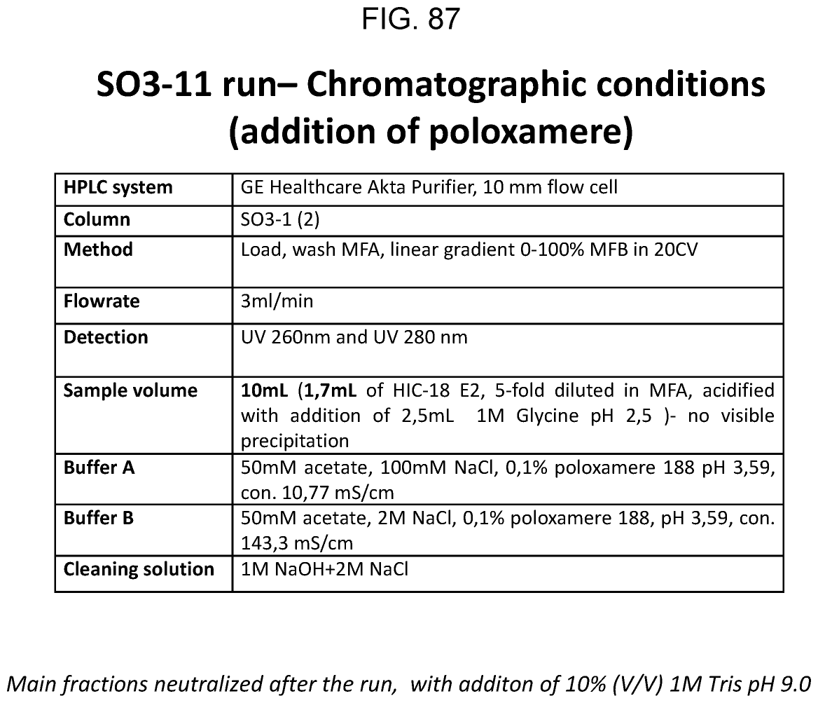

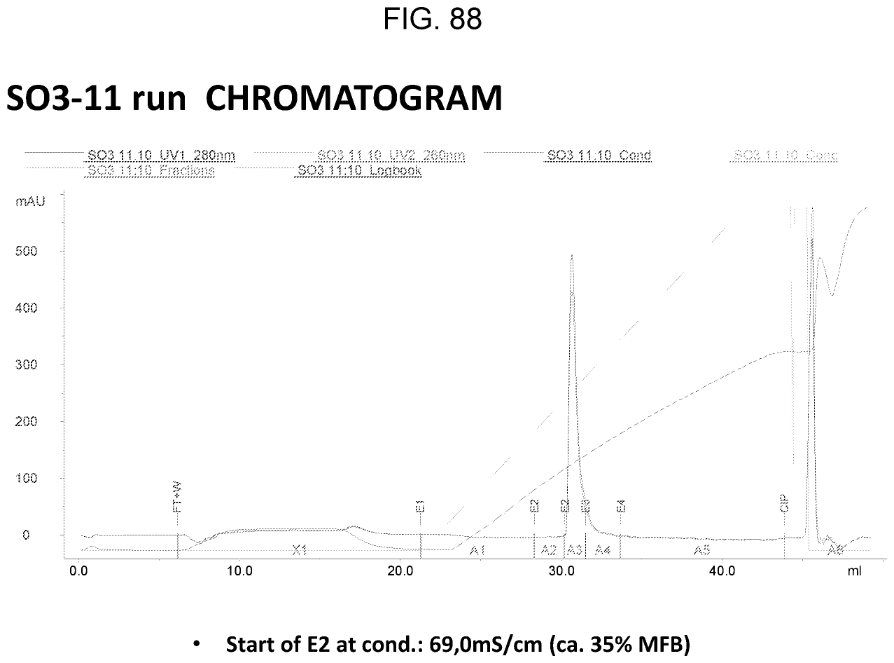

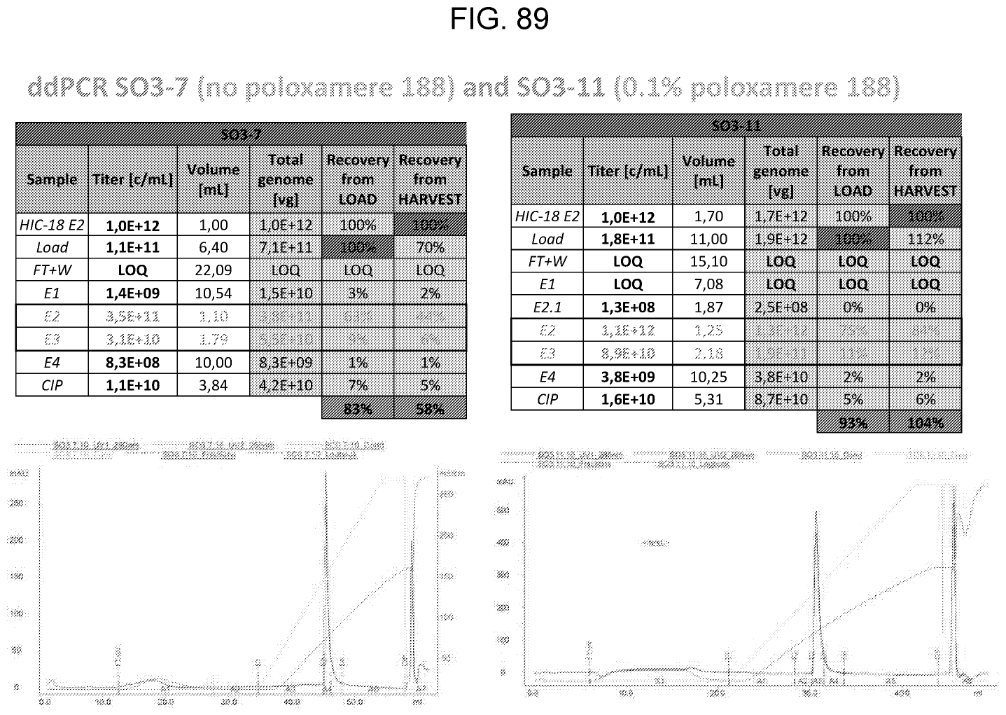

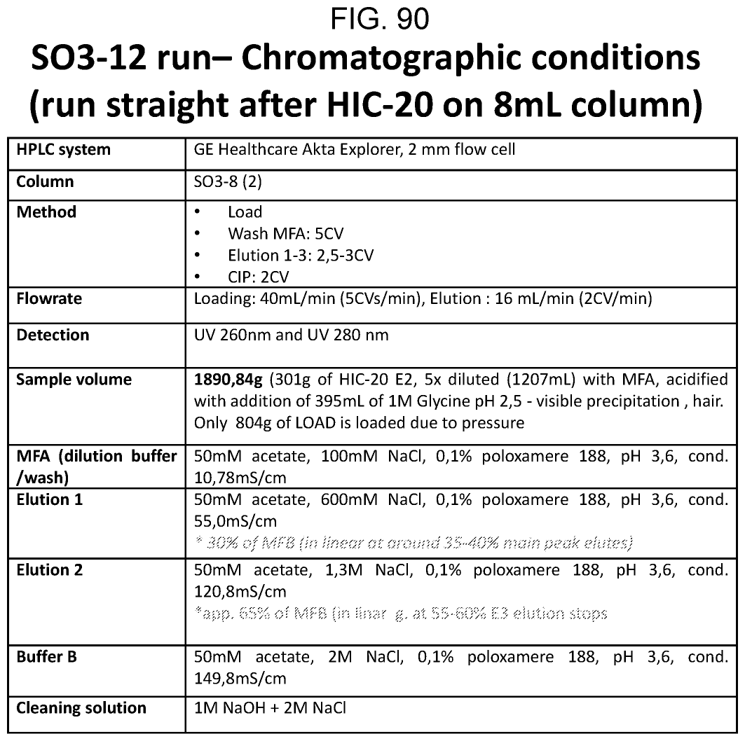

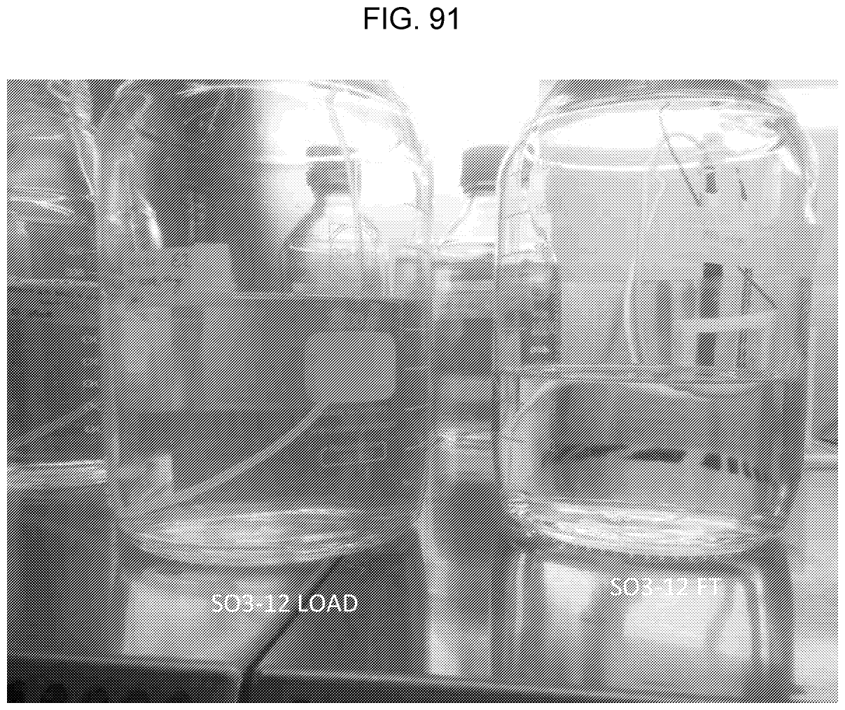

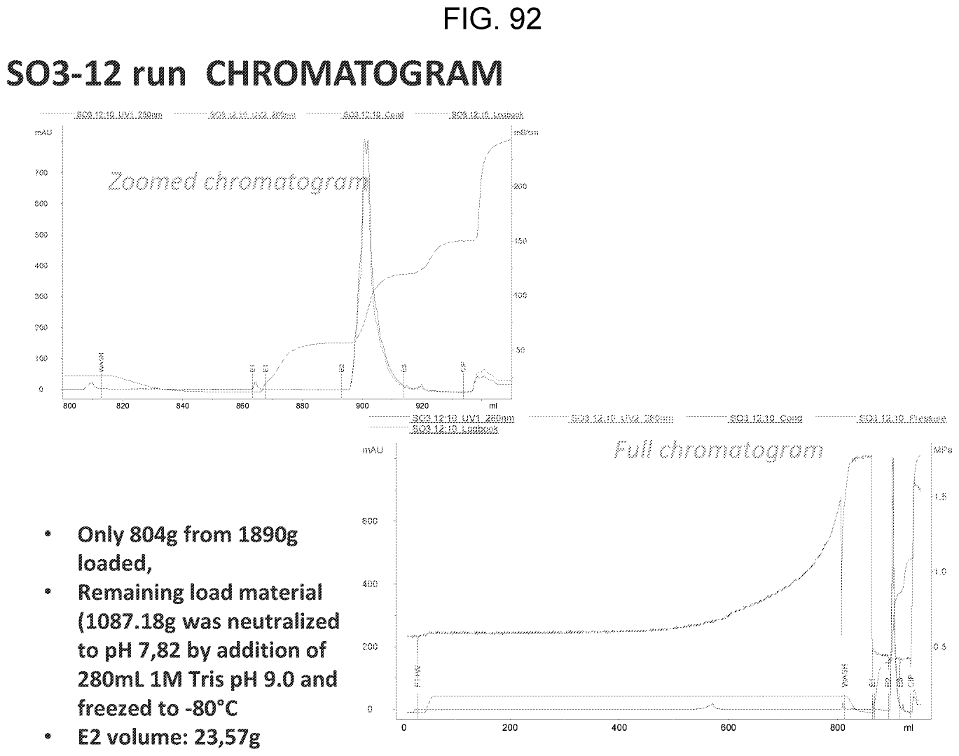

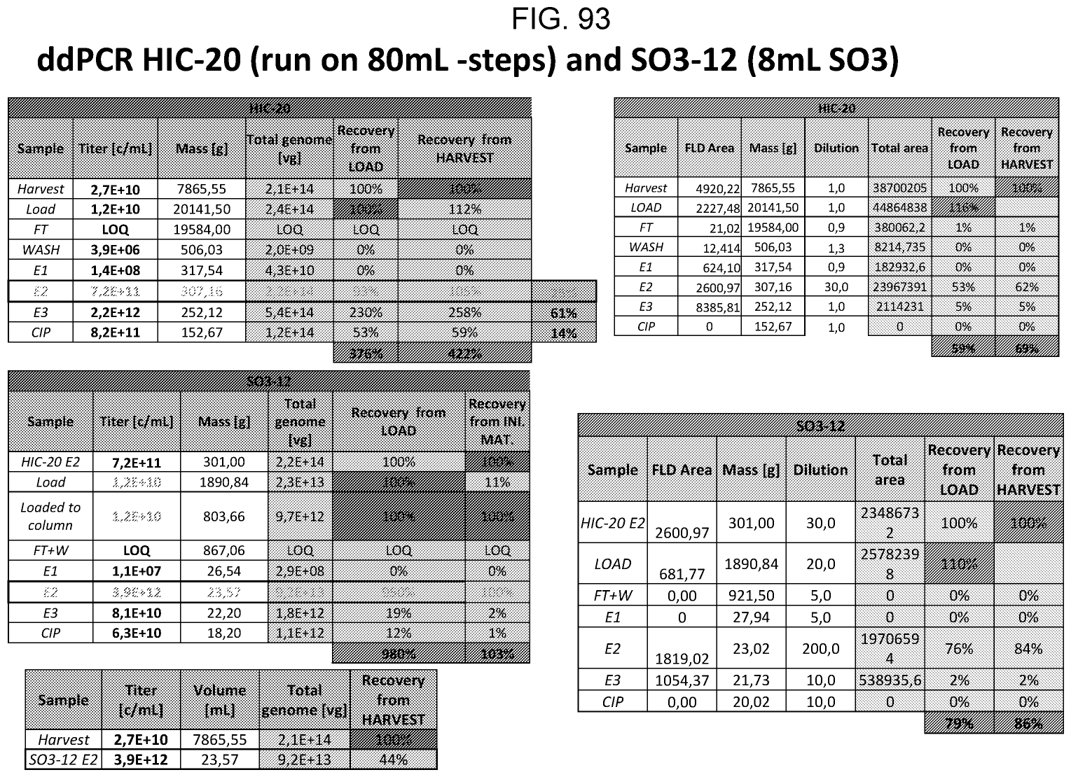

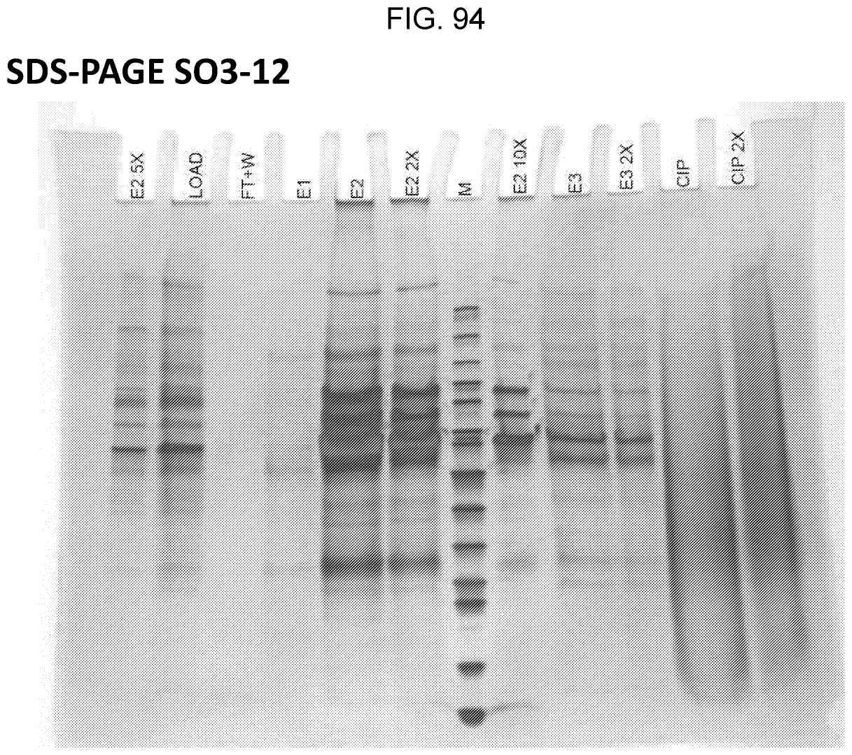

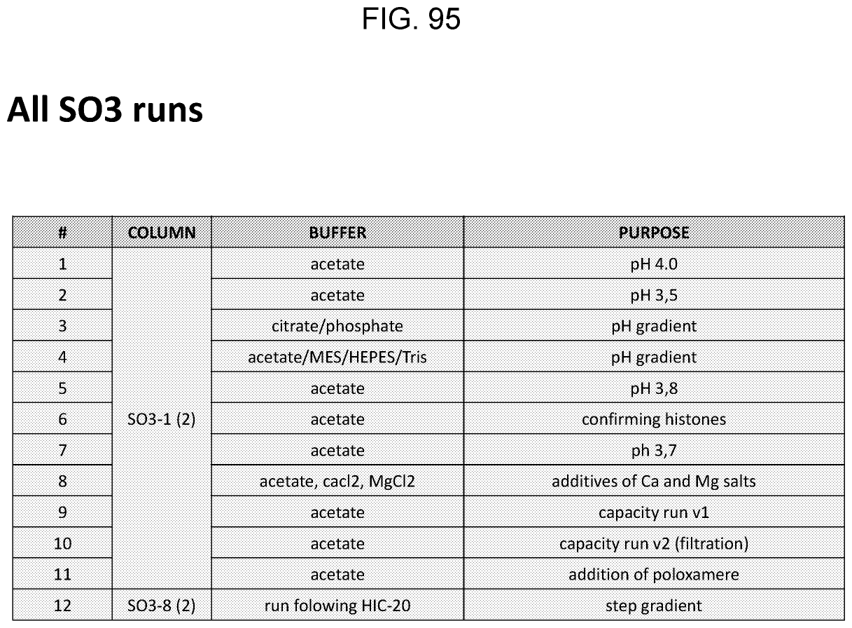

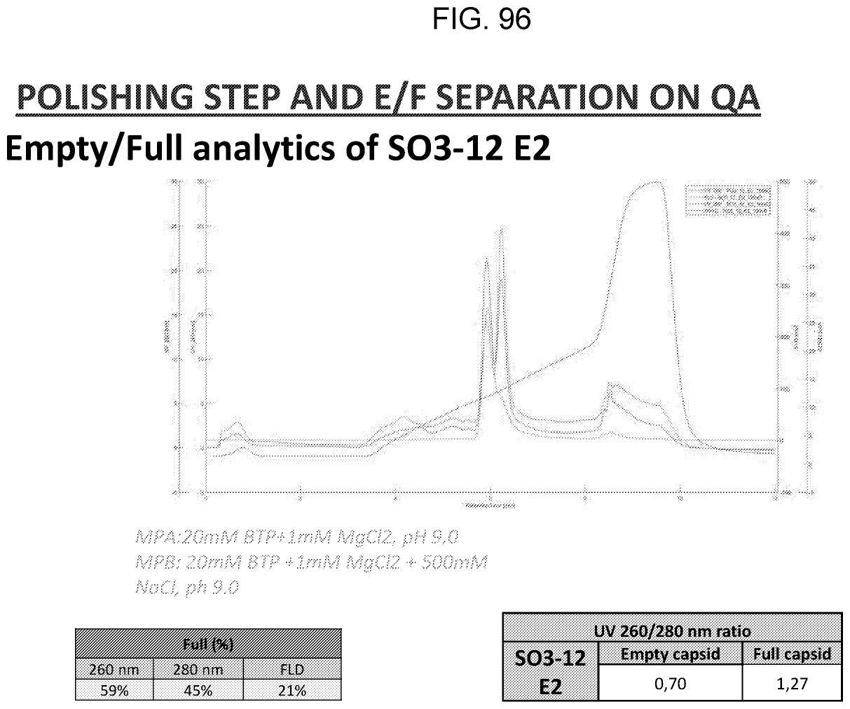

A process summary of an exemplary Cation Exchange Chromatography (CEX) step of the manufacturing processes of the disclosure is shown in FIG. 68.

In some embodiments of the methods of the disclosure, the CEX step comprises the steps of: (i) diluting the HIC eluate comprising a plurality of rAAV viral particles from the HIC step, and optionally, filtering the HIC eluate; (ii) loading the diluted HIC eluate on a CEX column; (iii) generating a CEX chromatogram; and (iv) selecting a fraction from the CEX chromatogram containing rAAV particles to produce the CEX eluate comprising a plurality of rAAV viral particles. In some embodiments, the CEX chromatography comprises an SO₃− cation exchange matrix in the chromatography column. In some embodiments, the chromatography column is an 80 mL monolith. In some embodiments, the HIC eluate is diluted into a low salt buffer prior to the CEX step. In some embodiments, the diluted HIC eluate is adjusted to pH 3.0-4.0. In some embodiments, the diluted HIC eluate is adjusted to pH 3.6+/−0.1. In some embodiments, this brings the pH below that of pI of the rAAV viral particles, producing positively charged rAAV viral particles. In some embodiments, a step gradient is used to elute the rAAV particles. In some embodiments, the method further comprises neutralizing the pH of the CEX eluate. Illustrative buffer conditions are provided, e.g., in FIGS. 213 and 336.

### Anion Exchange Chromatography

In some embodiments of the methods of the disclosure, Anion Exchange Chromatography (AEX) can be used to enrich the plurality of rAAV particles for full rAAV particles. Full rAAV particles are AAV particles comprising a single stranded DNA comprising an AAV-Construct of the disclosure. In some embodiments, the full rAAV particles comprise a sequence encoding a 5′ ITR, a sequence encoding a GRK1 promoter, a sequence encoding RPGR^{ORF15}, a sequence encoding a BGH polyA signal and a sequence encoding a 3′ ITR. In some embodiments, the full rAAV particles comprise a sequence encoding a 5′ ITR, a sequence encoding ABCA4 or a portion thereof and a sequence encoding a 3′ ITR.

AEX is a type of ion exchange chromatography which separates molecules based on net surface charge. Full, empty and damaged and/or aggregated AAV viral particles have different isoelectric points (pI). In some embodiments, full and empty particles are separated based on the differing charges of the particles. Full particles are slightly more negatively charged than empty particles due to the present of the DNA genome. In some embodiments, rAAV particles are diluted into solution with a pH that is higher than the pI of the AAV particles. In some embodiments, separation is further enhanced by the removal of MgCl₂ from the solutions.

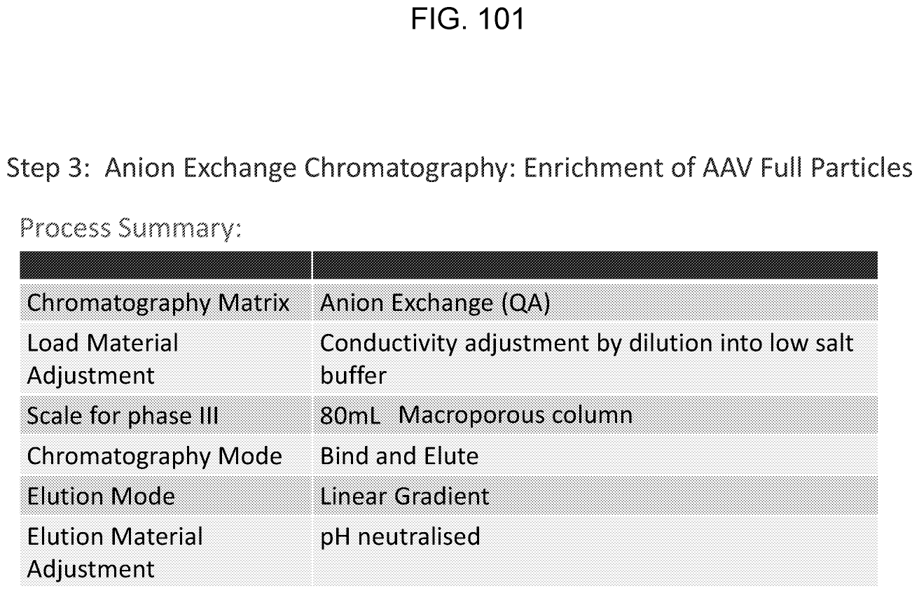

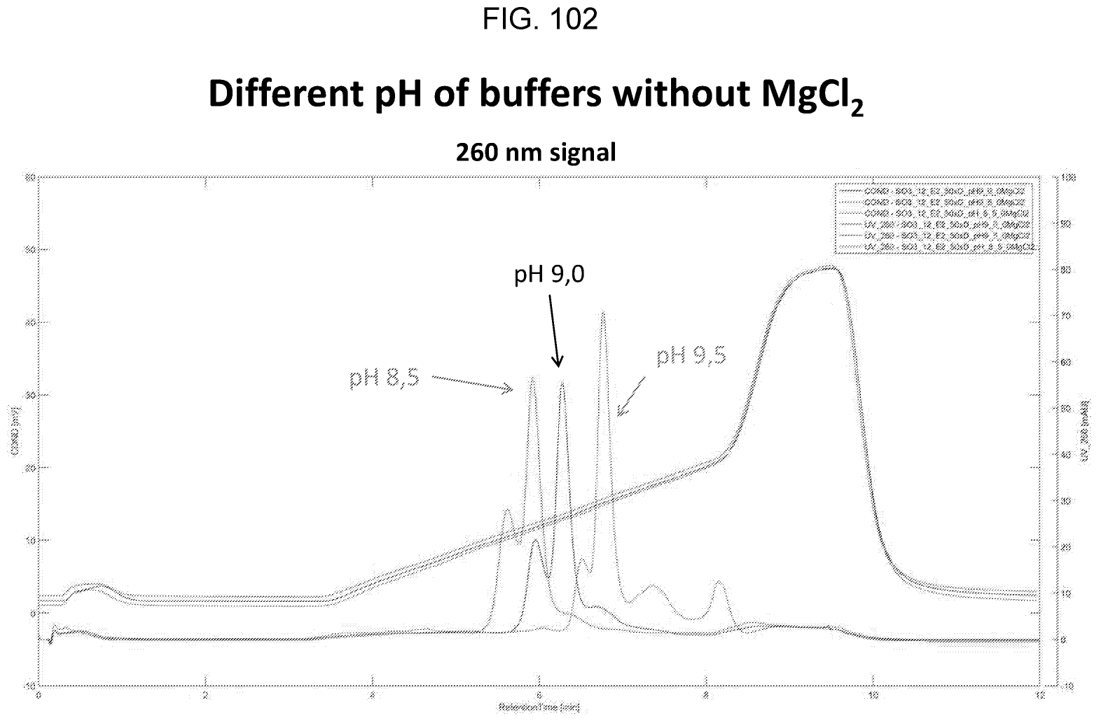

A process summary of an exemplary Anion Exchange Chromatography (AEX) step of the manufacturing processes of the disclosure is shown in FIG. 101.

In some embodiments of the methods of the disclosure, the AEX Chromatography step comprises the steps of: (i) diluting the CEX eluate comprising a plurality of rAAV viral particles; (ii) loading the diluted CEX eluate on a AEX column; (iii) generating an AEX chromatogram; and (iv) selecting a fraction from the AEX chromatogram containing full rAAV particles to produce the AEX eluate comprising a purified and enriched plurality of full rAAV particles. In some embodiments, the AEX chromatography comprises an Anion Exchange (QA) matrix in the chromatography column. In some embodiments, the column is an 80 mL macroporous matrix composition. In some embodiments, the CEX eluate is diluted into a low salt buffer prior to the AEX step. In some embodiments, the linear gradient is used to elute the full rAAV particles. In some embodiments, the method further comprises neutralizing the pH of the eluate comprising a purified and enriched plurality of full rAAV particles. Illustrative buffer conditions are provided, e.g., in FIGS. 213 and 336.

### TFF Concentration and Diafiltration

In some embodiments of the methods of the disclosure, the AEX eluate comprising a purified and enriched plurality of full rAAV particles is diafiltered and concentrated into a final formulation buffer (FFB) using Tangential Flow Filtration (TFF). Tangential Flow Filtration is a membrane filtration technique which can be classified as a microfiltration or ultrafiltration process, depending on membrane porosity in the specific TFF embodiment. In TFF, the feed stream passes parallel to the membrane face as one portion permeates the membrane, while the retentate is recirculated back to the reservoir. This process achieves volume reduction and additional purification using the principle of Tangential Flow Filtration (TFF).

This process utilizes diafiltration to formulate the AAV product into the desired Final Formulation buffer (20 mM Tris, pH 8, 1 mM MgCl₂, 200 mM NaCl, optionally with poloxamer at 0.001%).

Tangential flow filtration of the Elution product is conducted using a hollow fiber filter (HFF) cartridge with a molecular weight cut-off of 100 kDa (Spectrum). The cartridge and the system are equilibrated with Tris 20 mM, MgCl₂ 1 mM, NaCl 200 mM pH 8 buffer to obtain a pH 8.0±0.2 on the Permeate side.

In some embodiments of the methods of the disclosure, the method comprises a two TFF steps, both the first and second TFFs are performed using a 100 kDa HFF.

The product is concentrated to the minimum volume before the diafiltration in continuous mode against minimum 6 volumes of Formulation Buffer. The retentate is collected. The system is rinsed with Formulation Buffer. This rinse is collected in a different vessel.

If required for longer term storage (>60 days), in some nonlimiting examples of long term storage methods, the product is submicron filtered using a 0.2 μm filter. Once the Drug Substance is completely filtered, the filter is rinsed with the final formulation buffer.

After QC sampling, the purified bulk Drug Substance is stored at <−80° C. Optionally, poloxamer 188 is added to the Drug Substance prior to freezing and storage at <−80° C.

Method of Making a Drug Product from a Drug Substance

Compositions of the disclosure may be supplied as liquids. In some embodiments of the compositions of the disclosure, including those wherein the composition comprises a Drug Product, the Drug Product is supplied in sterile glass vials. In some embodiments, the sterile glass vials are sterile clear glass vials. In some embodiments, the sterile glass vials are capped with stoppers. In some embodiments, the stoppers are plastic. In some embodiments, the sterile glass vials are capped and further enclosed with overseals.

### Control of Drug Substances of the Disclosure

Exemplary Drug Substances are characterized by the tests listed in Table 8.

TABLE 8

| Test | Test Method |
|---|---|
| Physical Titre | qPCR or ddPCR Based DNase Resistant |
| | Particle (DRP) Assay |
| Infectious Unit (IU) Titre | Infection of RC32 cells followed by |
| | detection of AAV8 by qPCR |
| DRP:IU Ratio - Calculation | n/a |
| Total Particles | Commercial anti-AAV8 particle ELISA |
| Full:Empty Ratio | Transmission electron microscopy or AUC |
| Vector Identity (DNA) | Purification of vector DNA with DNA |
| | sequencing of both strands |
| Total Protein | Micro-BCA Protein Quantification |
| Purity | SDS-PAGE Assay with impurities |
| | estimated by intensity analysis |
| Replication Competent AAV | |
| HEK293 Host Cell Protein | Commercial ELISA Kit |
| Total DNA | Picogreen assay |
| HEK293 Host Cell DNA | qPCR assay |
| | Method res DNA SEQ Human (Life |
| | Technologies kit) |
| Residual BSA | Commercial ELISA Kit |
| Residual Benzo nase | Commercial ELISA Kit |
| Residual AVB | Commercial ELISA kit |
| Bioburden Assay | Membrane Filtration |
| Endotoxin Assay | Quantitative kinetic-chromogenic method. |

n/a: Not Applicable

Analytical Procedures

Physical Titre:

In some embodiments, the genomic titre is determined using qPCR. This method allows quantification of genomic copy number. Samples of the vector stock are diluted in buffer. The samples are DNase treated and the viral capsids lysed with proteinase K to release the genomic DNA. A dilution series is then made. Replicates of each sample are subjected to qPCR using a Taqman based Primer/Probe Set. A standard curve is produced by taking the average for each point in the linear range of the standard plasmid dilution series and plotting the log copy number against the average CT value for each point. In some embodiments, the plasmid DNA used in the standard curve is in the supercoiled conformation and in others it is in the linear conformation. Linearized plasmid can be prepared, for example by digestion with HindIII restriction enzyme, visualized by agarose gel electrophoresis and purified using the QIAquick Gel Extraction Kit (Qiagen) following manufacturer's instructions. Other restriction enzymes that cut within the plasmid used to generate the standard curve may also be appropriate. In some embodiments, the use of supercoiled plasmid as the standard increased the titre of the AAV vector compared to the use of linearized plasmid. The titre of the rAAV vector can be calculated from the standard curve and is expressed as DNase Resistant Particles (DRP)/mL.

In some embodiments, the genomic titre is determined using droplet digital PCR (ddPCR). A samples of AAV or the genomic DNA thereof may be fractionated into a plurality of nanoliter scaled droplets (e.g. 20,000 droplets) comprising an oil/water emulsion. The PCR occurs in each droplet of the plurality. This technique provides the advantage of requiring less sample and smaller volumes of reagents compared with reactions performed without the use of a droplet. Following PCR, each droplet is analyzed or read to determine the fraction of PCR-positive droplets in the original sample. These data are then analyzed using Poisson statistics to determine the target DNA template concentration in the original sample. During droplet generation, template molecules are distributed randomly into droplets. Some droplets contain no template, some contain one template molecule, and others contain more than one. Due to the random nature of the partitioning, the fluorescence data after amplification are well fit by a Poisson distribution. The number of positive droplets corresponds to the concentration of target sequence in the sample. The ddPCR system can accurately analyze samples in which multiple targets are amplified in the same droplet, thereby removing any requirement for one template per droplate at the beginning of a reaction or for one target per droplate following a reaction to quantify the target copies per droplet. ddPCR may use the same PCR reagents as standard PCR.

Infectious Unit (IU) Titre: This assay quantifies the number of infectious particles of AAV. Quantification is performed by infecting RC32 cells (HeLa expressing AAV8 Rep/Cap) with serial dilutions of the vector sample and uniform concentrations of wild type adenovirus to provide helper function. Several days post infection, the cells are lysed diluted to reduce PCR inhibitors and assayed by qPCR in the same manner as described in the physical titre assay above, except that the DNase and Proteinase K digestion is omitted and only the qPCR portion is performed. Individual wells are scored as Positive or Negative for AAV amplification. The scored wells are used to determine the TCID₅₀ in IU/mL using the Karber Method.

Total Particles: The assay uses an ELISA technique (AAV8 Titration ELISA KIT). A monoclonal antibody specific for a conformational epitope on assembled AAV8 capsids is coated onto microtitre strips and is used to capture AAV8 particles from the specimen. Captured AAV particles are detected in two steps. First a biotin-conjugated monoclonal antibody to AAV8 is bound to the immune complex. In the second step streptavidin peroxidase conjugate reacts with the biotin molecules. Addition of substrate solution results in a color reaction which is proportional to the amount of specifically bound viral particles. The absorbance is measured photometrically at 450 nm.

Full:empty Ratio (Transmission Electron Microscopy): The full:empty ratio of AAV2 particles may be determined using negative staining transmission electron microscopy (TEM). Samples are applied to a grid fixed. Samples are visualized using a transmission electron microscope and counts are performed of the full (i.e. containing DNA) and empty AAV2 capsid particles based on their morphology. The ratio of full:empty particles is calculated from the particle counts.

Full:empty Ratio (Analytical Ultracentrifugation): The full:empty ratio of AAV8 particles may be determined using analytical ultracentrifugation (AUC). AUC has an advantage over other methods of being non-destructive, meaning that samples may be recovered following AUC for additional testing. Samples comprising empty and full AAV8 particles are applied to a liquid composition through which the AAV8 move during an ultracentrifugation. A measurement of sedimentation velocity of one or more AAV8 particles provides hydrodynamic information about the size and shape of the AAV particles. A measure of sedimentation equilibrium provides thermodynamic information about the solution molar masses, stoichiometries, association constants, and solution nonideality of the AAV8 particles. Exemplary measurements acquired during AUC are radial concentration distributions, or “scans”. In some embodiments, scans are acquired at intervals ranging from minutes (for velocity sedimentation) to hours (for equilibrium sedimentation). The scans of the methods of the disclosure may contain optical measurements (e.g. light absorbance, interference and/or fluorescence). Ultracentrifugation speeds may range from between 10,000 rotations per minute (rpm) and 75,000 rpm, inclusive of the endpoints. As full AAV8 particles and empty AAV8 particles demonstrate distinct measurements by AUC, the full/empty ratio of a sample may be determined using this method.

Vector Identity (DNA): This assay provides a confirmation of the viral DNA sequence. The assay is performed by digesting the viral capsid and purifying the viral DNA. The DNA is sequenced with a minimum of 2 fold coverage both forward and reverse where possible (some regions, e.g., ITRs are problematic to sequence). The DNA sequencing contig is compared to the expected sequences to confirm identity.

Total Protein: This assay quantifies the total amount of protein present in the test article by using a Micro-BCA kit. In order to eliminate matrix effects of the formulation buffer samples are precipitated with acetone and the precipitated protein re-suspended in an equal volume of water prior to analysis. The protein concentration determination is performed by mixing test article or diluted test article with a Micro-BCA reagent provided in the kit. The same is performed using dilutions of a Bovine Serum Albumin (BSA) Standard. The mixtures are incubated at 60° C. and the absorbance measured at 562 nm. A standard curve is generated from the standard absorbance and the known concentrations using a linear regression fit. The unknown samples are quantified according to the linear regression.

Purity: This assay provides a semi-quantitative determination of AAV purity. Based on the results of the AAV8 capsid particle ELISA, samples are concentrated by SpeedVac and either 4×10\hat{ }10 or 1×10\hat{ }11 particles are loaded and the capsid proteins are separated on an SDS-PAGE gel. Densitometry analysis of the SYPRO Orange stained gels allows calculation of the approximate impurity levels relative to the capsid proteins (Vp1, Vp2 and Vp3).

Replication Competent AAV: Test article is used to transduce HEK293 cells in the presence or the absence of wild type adenovirus. Three successive rounds of cell amplification will be conducted and total genomic DNA is extracted at each amplification step.

The rcAAV8 are detected by real-time quantitative PCR. Two sequences are isolated genomic DNA; one specific to the AAV2 Rep gene and one specific to an endogenous gene of the HEK293 cells (human albumin). The relative copy number of the Rep gene per cell is determined. The positive control is the wild type AAV virus serotype 8 tested alone or in the presence of the rAAV vector preparation.

The limit of detection of the assay is challenged for each tested batch. The limit of detection is “X” rcAAV per “Y” genome copies of test sample. If a test sample is negative for Rep sequence, the result for this sample will be reported as: NO REPLICATION, <“X” rcAAV per “Y” genome copies of test sample. If a test sample is positive for Rep sequence, the result for this sample will be reported as: REPLICATION, >“X” rcAAV per “Y” genome copies of test sample.

HEK293 Host Cell Protein: The HEK293 host cell protein (HCP) assay is an immunoenzymetric assay. Samples of purified virus are reacted in microtitre strips coated with an affinity purified capture antibody. A secondary horseradish peroxidase (HRP) conjugated enzyme is reacted simultaneously, resulting in the formation of a sandwich complex of solid phase antibody-HCP-enzyme labelled antibody. The microtitre strips are washed to remove any unbound reactants. The quantity of HEK293 HCPs is detected by the addition of 3,3′,5,5′ tetramethyl benzidine peroxidase, an HRP substrate, to each well. The amount of hydrolyzed substrate is read on a plate reader and is directly proportional to the concentration of HEK293 HCPs present.

Total DNA: Picogreen reagent is an ultra-sensitive fluorescent nucleic acid stain that binds double-stranded DNA and forms a highly luminescent complex (λexcitation=480 nm-λemission=520 nm). This fluorescence emission intensity is proportional to dsDNA quantity in solution. Using a DNA standard curve with known concentrations, DNA content in test samples is obtained by converting measured fluorescence.

HEK293 Host Cell DNA: The original process measured size and quantity of 3 different amplicons whereas the improved process measures total hcDNA including high molecular weight and sheared DNA. The qualification data the improved process demonstrates that the assay is specific and sufficiently sensitive to meet the requirements in assessing hcDNA per dose of <10 ng/dose (WHO Expert Committee on Biological Standardization, 2013).

Residual BSA: Residual BSA is quantified using a commercially available ELISA kit manufactured and marketed by Bethyl. The scientific principle to the ELISA kit is very similar to that specified for the Host Cell Protein ELISA.

Residual Benzonase: This assay uses purified polyclonal antibodies specific to Benzonase endonuclease to detect residual Benzonase in the test sample by sandwich ELISA. Accurate measurement is achieved by comparing the signal of the sample to the Benzonase endonuclease standards assayed at the same time.

Bioburden Assay: This procedure is used to determine quantitatively (if detectable) the amount of bioburden present in a sample. The method used involves membrane filtration of half of the sample onto each of two membranes. The membranes are placed onto separate agar media plates which are incubated in aerobic and anaerobic conditions sequentially at 20-25° C. and 30-35° C. At the conclusion of incubation; aerobe, anaerobe, and fungal counts are expressed as CFU/mL of sample.

Endotoxin Assay: This assay is used to determine if bacterial endotoxins are present in the test article. A quantitative procedure is performed by the kinetic-chromogenic method. Known amounts of endotoxin are tested in parallel with the test article for an accurate determination of the level of bacterial endotoxin. The potential for interference by the test article is examined by spiking the test article plus LAL reagent with specified levels of endotoxin. Following the inhibition/enhancement test, the endotoxin content of the test article is determined.

Quantitative PCR (qPCR): qPCR can be used to confirm HPLC chromatogram results (also referred to as real-time PCR or reverse-transcription PCR, both abbreviated as RT-PCR). qPCR uses polymerase (e.g. a Taq polymerase) in a standard PCR reaction to amplify a target DNA fragment from a complex sample using a pre-validated primer or primer/probe assay. The PCR reaction uses a fluorescent reporter to measure the generation of amplified DNA at every cycle of PCR, thereby providing either an absolute or relative measure of DNA quantity. When the DNA is in the log linear phase of amplification, the amount of fluorescence produced by the PCR increases above the background. The point at which the fluorescence becomes measurable is called the threshold cycle (CT) or crossing point. By comparing the CT of the test sample to a known sample or a standard curve (using a series of dilutions of a known sample), the amount of DNA in the test sample can be determined. In preferred embodiments, the amount of sample/test DNA is compared against an invariant or endogenous gene of the host cell (e.g. a housekeeping gene including but not limited to β-actin).

Droplet Digital PCR (ddPCR): ddPCR can be used to confirm HPLC chromatogram results. ddPCR uses Taq polymerase in a standard PCR reaction to amplify a target DNA fragment from a complex sample using a pre-validated primer or primer/probe assay. The PCR reaction is partitioned into thousands of individual reaction vessels prior to amplification, and the data is acquired at the reaction end point. ddPCR offers direct and independent quantification of DNA without standard curves, and can give a precise and reproducible data. End point measurement enables nucleic acid quantitation independent of reaction efficiency. ddPCR can be used for extremely low target quantitation from variably contaminated samples.

### Stability of AAV Compositions

Compositions of the disclosure maintain long term stability when stored at <−60° C. For example, compositions of the disclosure maintain long term stability when stored at temperature between −80° C. and 40° C. (approximately human body temperature), inclusive of the endpoints. For example, compositions of the disclosure maintain long term stability when stored at temperature between −80° C. and 5° C., inclusive of the endpoints. For example, compositions of the disclosure maintain long term stability when stored at −80° C., −20° C. or 5° C. In some embodiments, compositions of the disclosure are formulated as liquids or suspensions, aliquotted into one or more containers (e.g. vials), and stored at <−60° C. In some embodiments, compositions of the disclosure are formulated as liquids or suspensions, aliquotted into one or more containers (e.g. vials), and stored at −80° C., −20° C. or 5° C.

Compositions of the disclosure may be provided in a container with an optimal surface area to volume ratio for maintaining long term stability when stored at <−60° C. Compositions of the disclosure may be provided in a container with an optimal surface area to volume ratio for maintaining long term stability when stored at −80° C., −20° C. or 5° C. In some embodiments, compositions of the disclosure are formulated as liquids or suspensions, aliquotted into one or more containers (e.g. vials), and stored in one or more containers with a surface area to volume ratio as large as possible when all storage requirements are considered.

Compositions of the disclosure maintain long term stability when stored at ambient relative humidity.

### EXAMPLES

### Example 1

### Development of the Purification Process

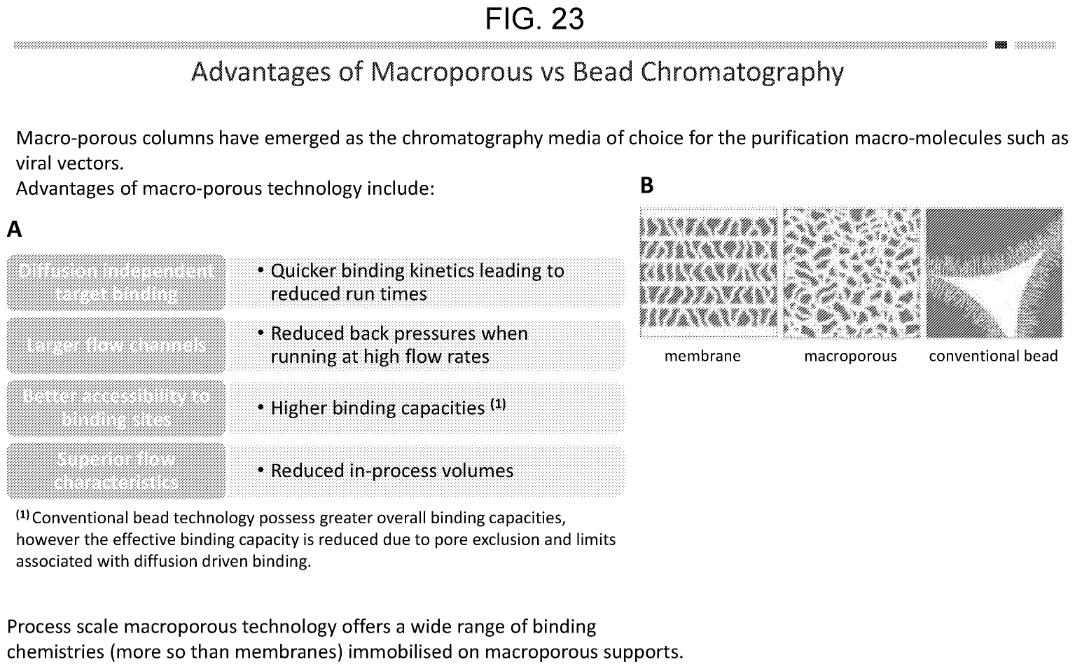

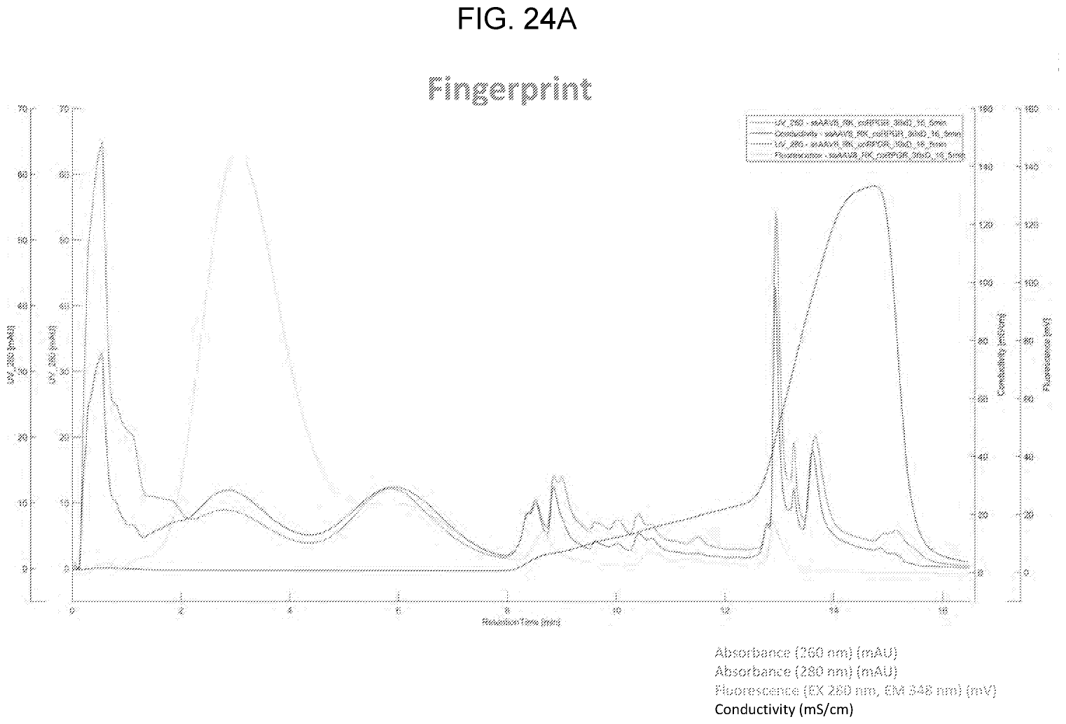

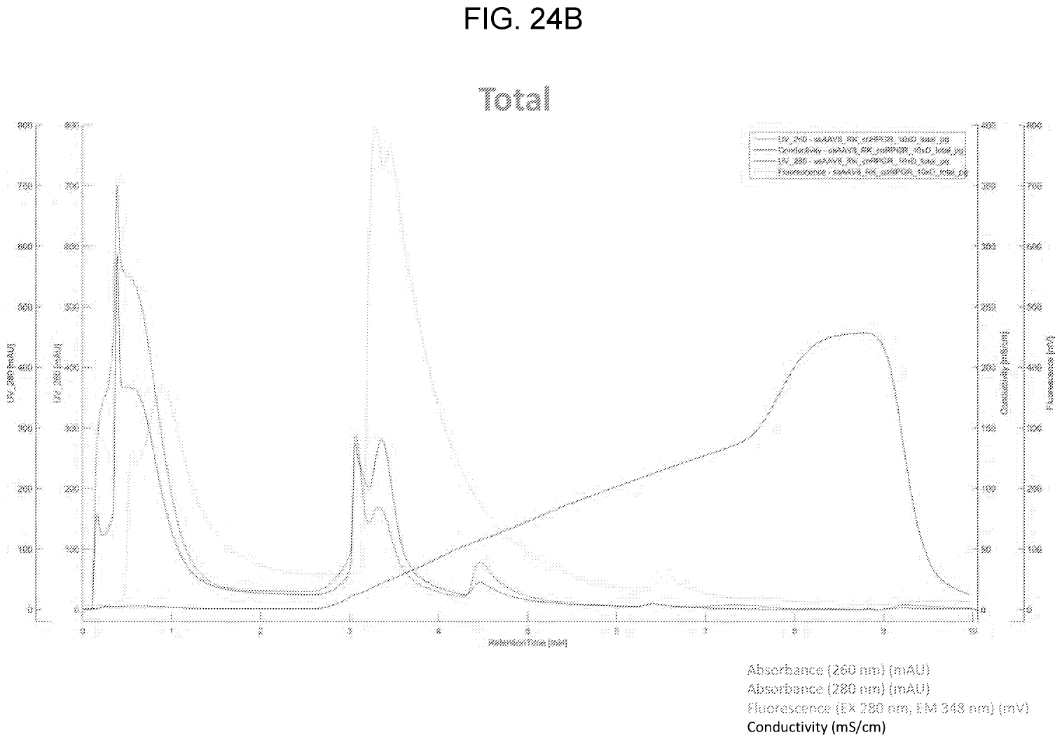

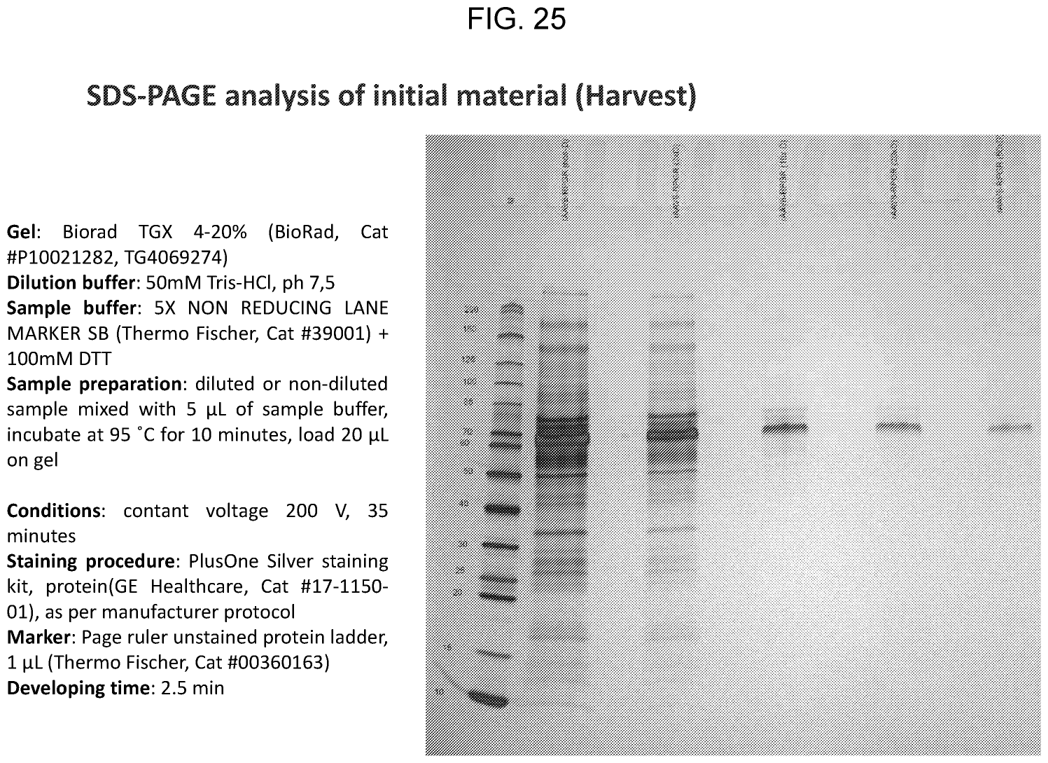

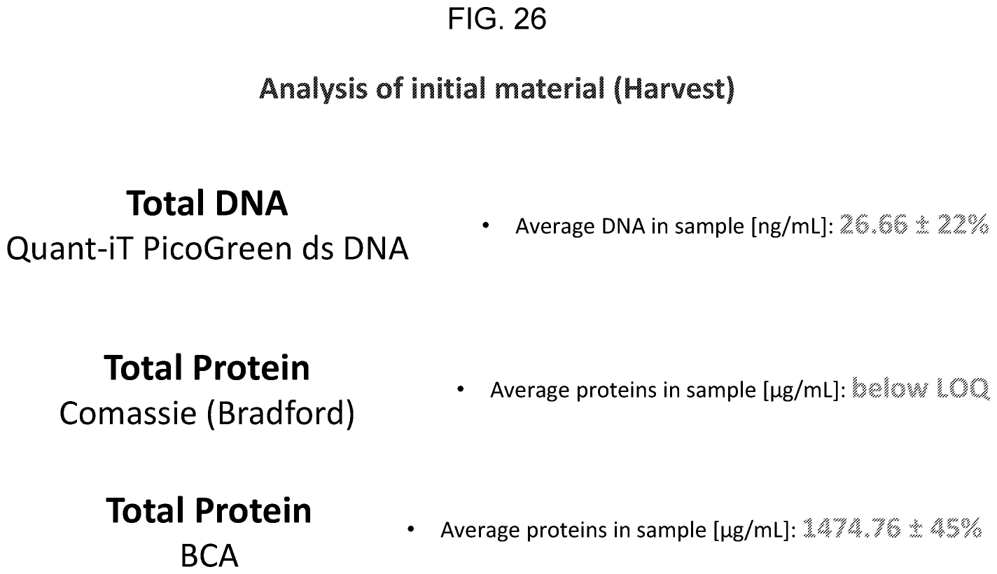

FIG. 23 shows a comparison of monoliths versus bead chromatography. Macro-porous columns (membranes and monoliths) have emerged as the chromatography media of choice for the purification of macromolecules such as rAAV viral vectors. The advantages of macro-porous technology over conventional beads include, but are not limited to: diffusion independent target binding, leading to quicker binding kinetics and reduced run times; larger flow channels, leading to reduced back pressures when running at high flow rates; better accessibility to binding sites, resulting in higher binding capacities; and superior flow characteristics, leading to reduced in-process volumes. While conventional bead technology possesses greater overall binding capacities, the effective binding capacity is reduced due to pore exclusion and limits associated with diffusion driven binding. Thus, macro-porous technology is a superior method for the purification of rAAV viral particles. In addition, process scale monolith technologies offers a wide range of binding chemistries (more so than membranes) immobilized on monolithic supports.

Total particle high performance liquid chromatography (HPLC) chromatogram the preferred method for screening purposes as it has a quick turnaround time (<24 hours). This method is the main reporting assay for the recovery determination of experiments. Verification of HPLC results are confirmed with Droplet Digital PCR (ddPCR) measurements.

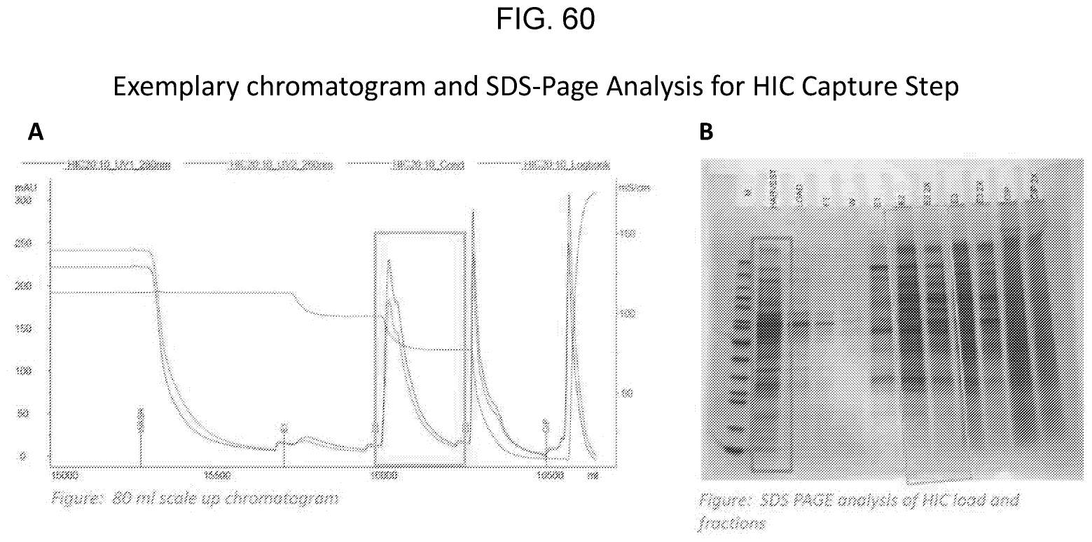

FIG. 60 shows an exemplary HIC capture step that has been scaled up from a 1 mL column to an 80 mL column. In FIG. 60A, the wash, E1, E2, E3 and clean in place (CIP) fractions are indicated on the x axis, and absorbance (in mAU) is indicated on the y-axis. AAV particles elute in fraction E2, indicated by the green boxes in FIG. 60A-B. Sensitivity analysis with regards to the elution conditions has demonstrated that this is a robust unit operation. FIG. 60B shows an SDS-PAGE analysis of the Harvest Media, flow through, wash, eluted fractions and CIP. Fraction E2 is indicated by the green box. A good correlation was observed between the ddPCR and the total particle HPLC method. Recoveries of >80% were expected for this unit operation.

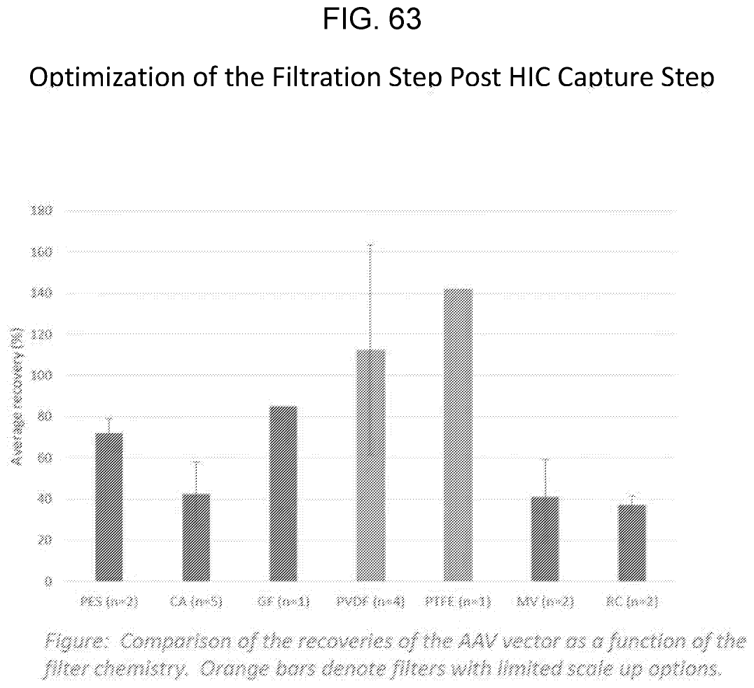

Following HIC capture, proteinaceous hair-like material was observed in the HIC eluate which led to unsustainable pressure increase during the subsequent chromatography step. A 0.45 μm cellulose acetate (CA) filter was used to retain the fibers but this led to a loss of 50% of the vector. Subsequently, filters were screened for rAAV retention to find a suitable alternative (FIG. 63). A 0.8/0.45 μm polyethersulfone (PES) combination filter was chosen for the filtration of the HIC eluate. Minimal losses were observed after implementation of this filter. The choice of filter was based on both filter recovery and filter availability at larger scales.

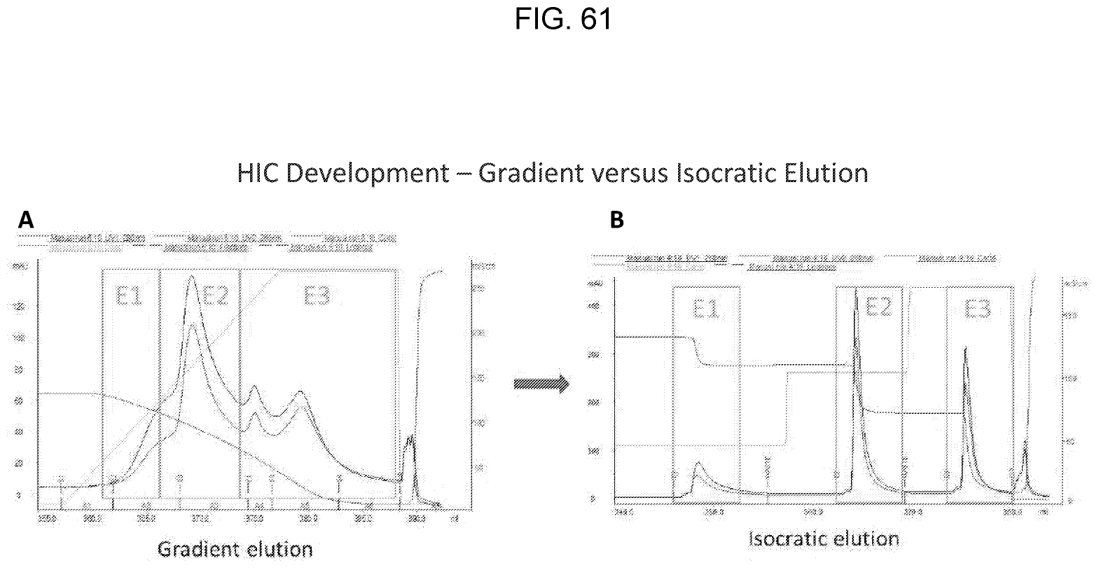

Both the gradient elution and the isocratic elution methods were tested for the HIC step (FIG. 61). Transforming a gradient elution to an isocratic elution was successful. In some embodiments, the isocratic elution is the preferred method for scaling up, as it is a more robust method. However, a complete partition of the eluted species may not be possible with the isocratic elution strategy. In some embodiments, a gradient elution is preferred.

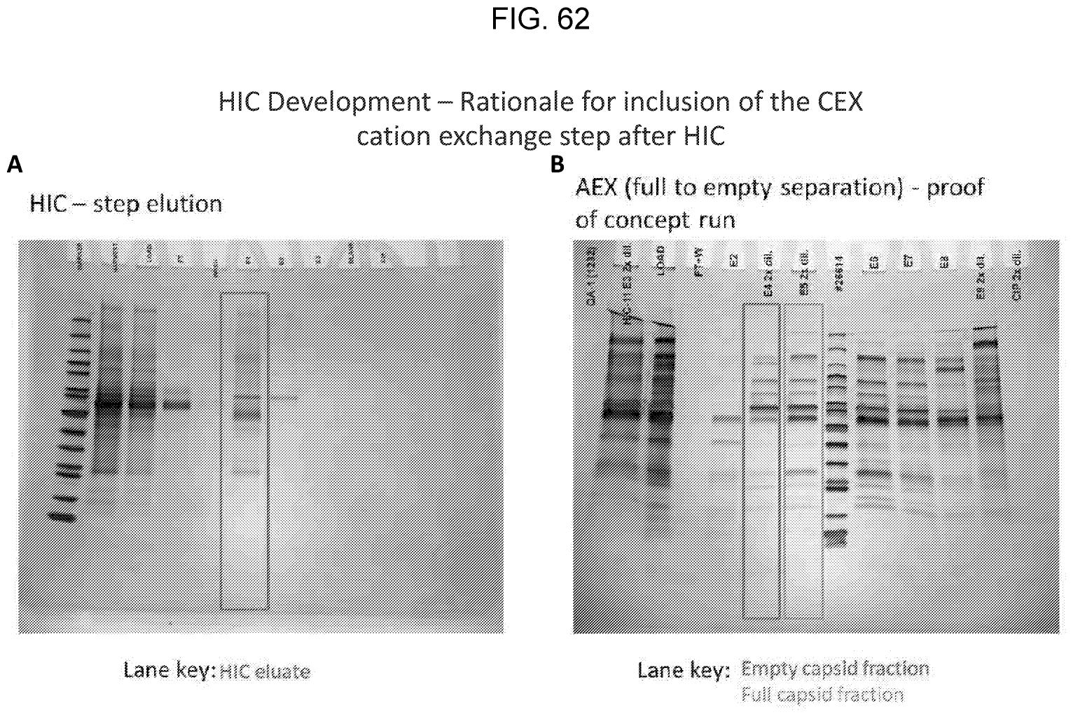

HIC development confirmed the need for a three step process including an intermediate CEX SO₃− polishing step (FIG. 62). The purity over the HIC step and the subsequent purity of the HIC (FIG. 62A) and AEX QA purified product (FIG. 62B) is not sufficient. The intermediate polishing step (CEX cation exchange, SO₃−) is required.

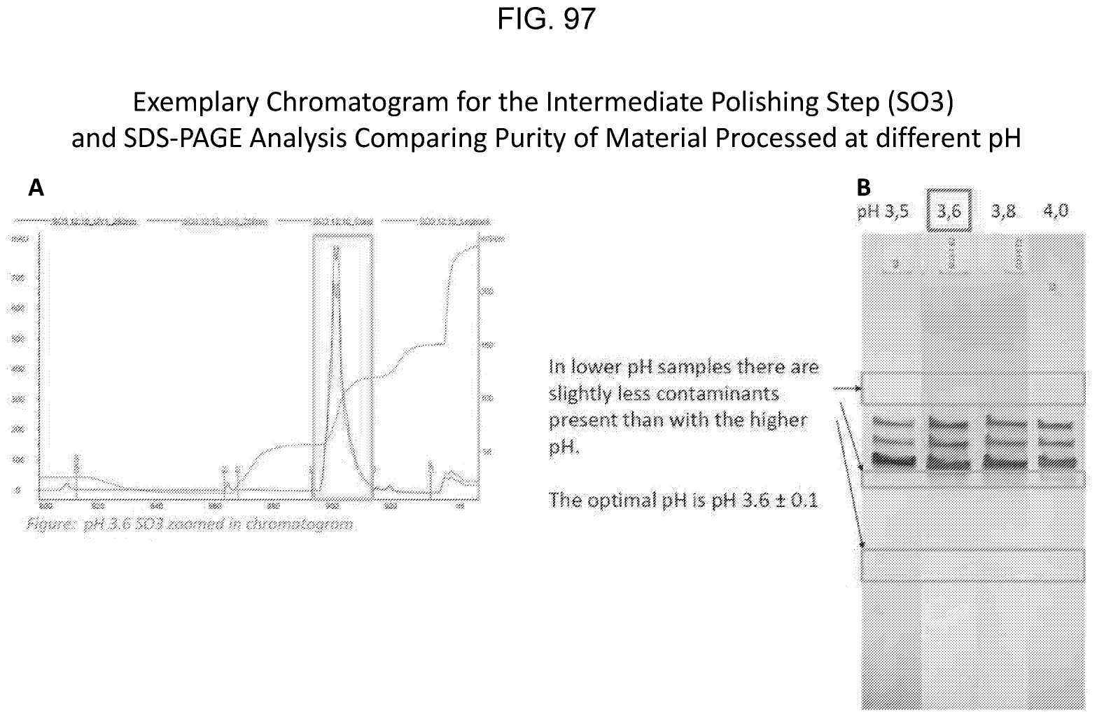

FIG. 97A shows a chromatogram of an exemplary intermediate polishing step using CEX and an SO₃− column matrix. A good correlation was observed between the ddPCR and the total particle HPLC methods. Recoveries of >80% were expected for this unit operation. FIG. 97B shows an SDS-PAGE gel comparing the AAV particle containing fractions of four different CEX intermediate polishing runs, where pH was adjusted to pH 3.5, pH 3.6, pH 4.0 and pH 4.0. All gels were slightly overdeveloped in order to expose all the protein bands present in the sample. The lower pH samples contained slightly less contaminants (orange boxes) than the higher pH samples. The optimal pH was pH 3.6+/−0.1.

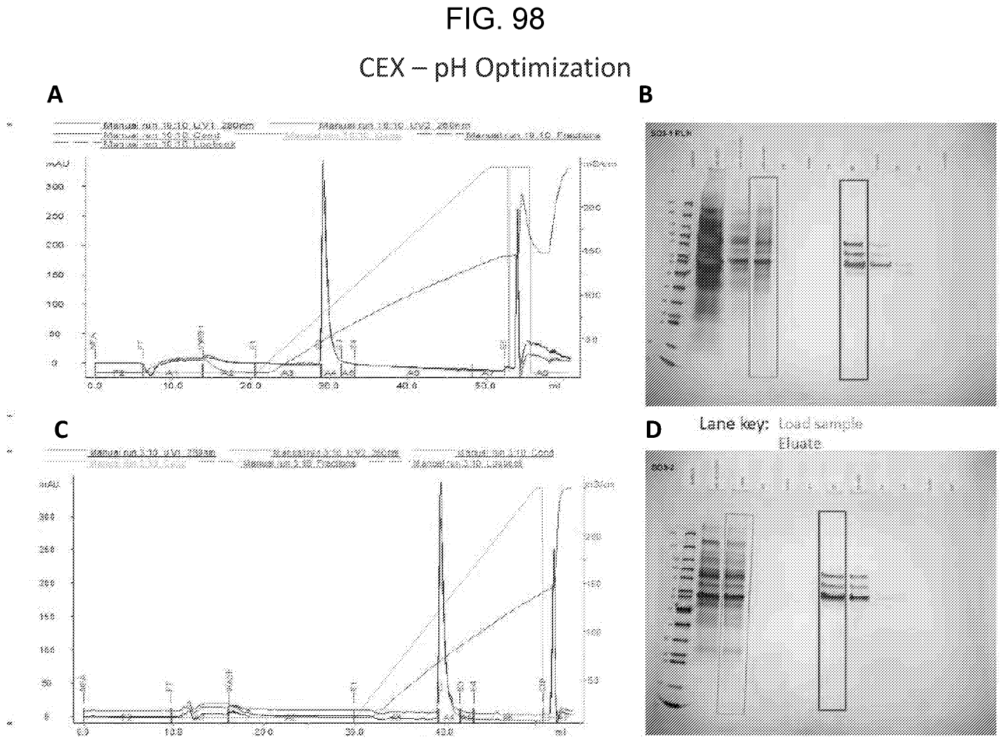

FIG. 98 shows two exemplary CEX chromatograms and corresponding SDS-PAGE gels, one with pH 4.0 (top) and the other with pH 3.5 (bottom). Higher purification was seen with pH 3.5 than pH 4.0. The use of the CEX intermediate polishing step increased purity to an appropriate level. DNA was separated out in the flow through fraction (FT), whereas protein impurities were retained on the column. The use of the lower pH (3.5) improved the purification factor. This is due to an increase in affinity, to the column, of proteins with low isoelectric points such as those found in AAV particles.

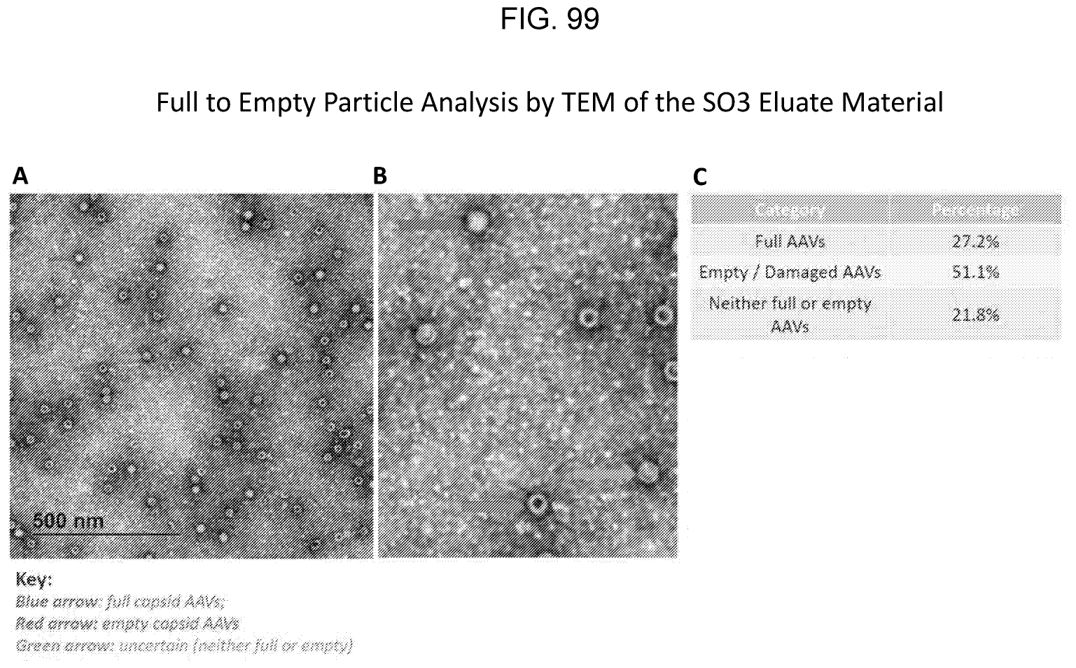

A TEM analysis of an exemplary CEX SO₃− eluate containing rAAV particles revealed that 27.2% of the particles were full, and 51.1% of the particles were empty or damaged (FIG. 99). 21.8% of the particles could not be classified as full or empty. The surface of these particles was not evenly bright, some dark spots were present, and they exhibited a grey circle with a white spot in the middle. These particles were presumed to not be entirely empty. This material was generated from genome plasmids with comprised 3′ ITR regions. The 3′ ITR may have affected encapsidation. Further development work for separating full and empty particles will used new material with a different 3′ ITR.

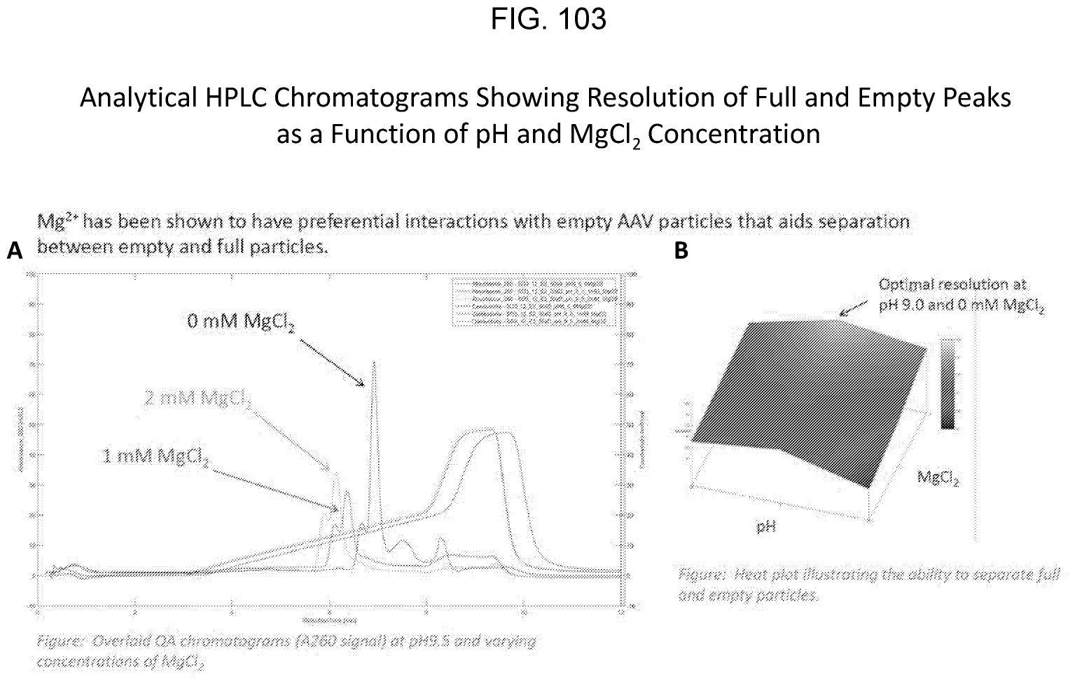

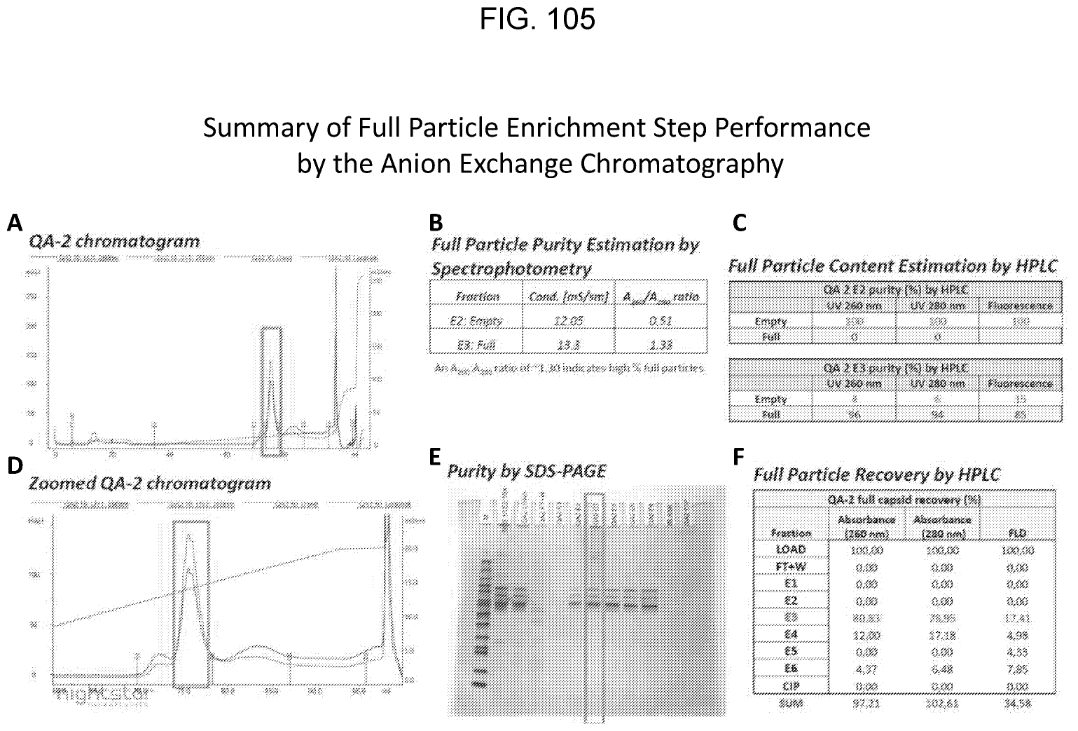

Optimal resolution of full and empty peaks depends on pH the concentration of MgCl₂. MG²⁺ has been shown to have preferential interactions with empty AAV particles that aids separation between empty and full particles. FIG. 103A shows an overlay of chromatograms generated by running CEX eluates on an AEX QA column at pH 9.5 and varying the concentrations of MgCl₂. The sharpest separation was seen at 0 mM MgCl₂ (black arrow and line). FIG. 103A shows a heat plot, illustrating that optimal full to empty separation at the AEX step occurs at pH 9.0 and 0 mM MgCl₂. A high percentage of full particles were recovered in the AEX E3 fraction (FIG. 105). By HPLC, an estimated 96% of the particles recovered in the E3 eluate were full, and 78-81% of full particles were recovered in the E3 eluate.

### Example 2: Purification of rAAV Particles

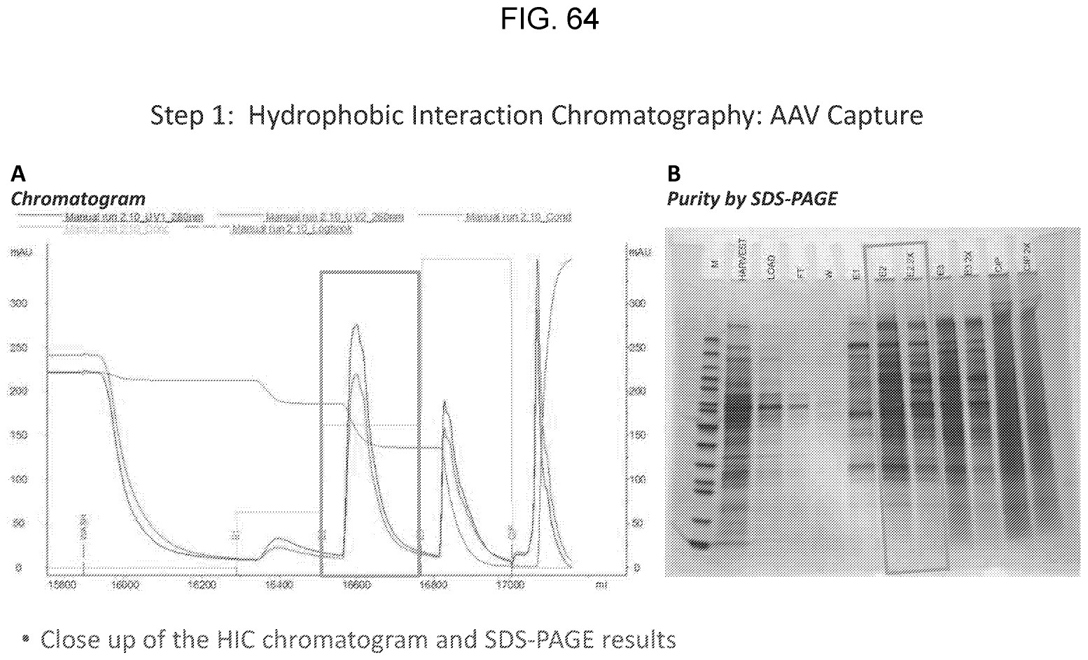

An exemplary HIC chromatogram showing the purification of rAAV particles of the disclosure from Harvest Media is shown in FIG. 64. Harvest Media comprising rAAV particles was diluted into high salt buffer and run on a 800 mL HIC monolith with a Hydrophobic Interaction (OH) matrix using the Bind and Elute Chromatography Mode. rAAV particles were eluted using a step-wise gradient. The chromatogram in FIG. 64A shows that rAAV particles are eluted in the E2 and E3 fractions, which are boxed. FIG. 64B shows an SDS-PAGE gel of the fractions recovered from the HIC step in FIG. 64A, showing, from left to right, a marker, the Harvest Media, Load, flow through (FT), wash (W), fractions E1, E2, E2 diluted two-fold (E2.2×), E3, E3 diluted two-fold (E3.2×), the clean in place step (CIP), and the CIP diluted two-fold (CIP.2×). E2 and E2.2× contain rAAV particles and are boxed.

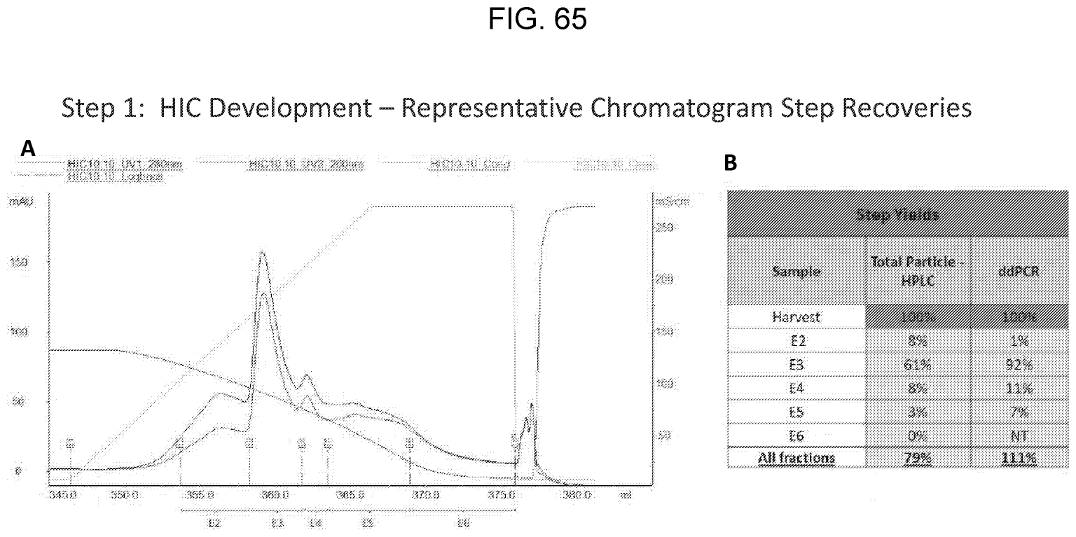

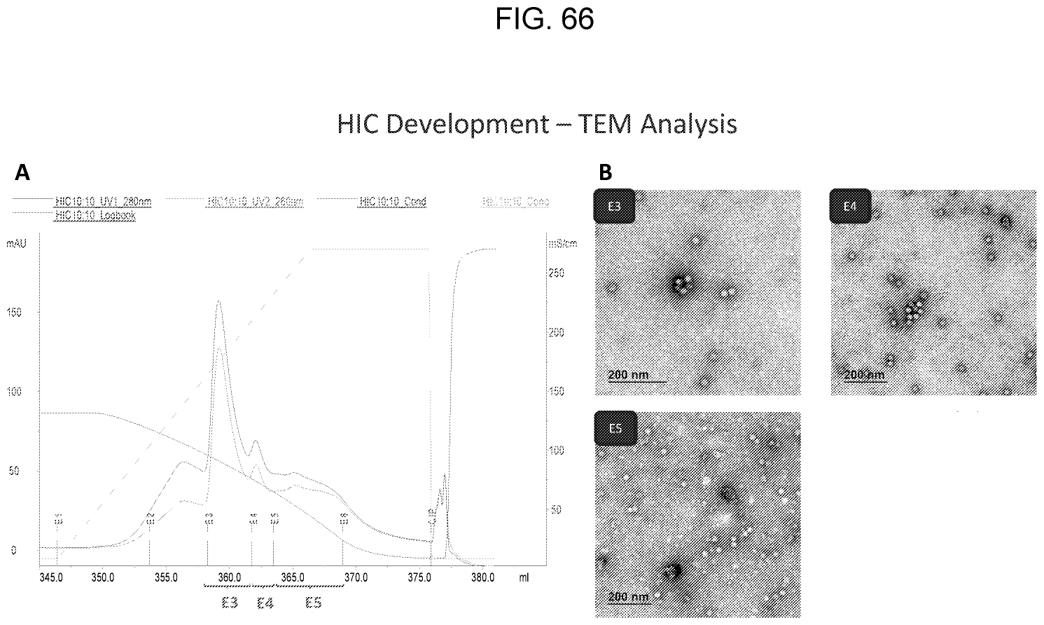

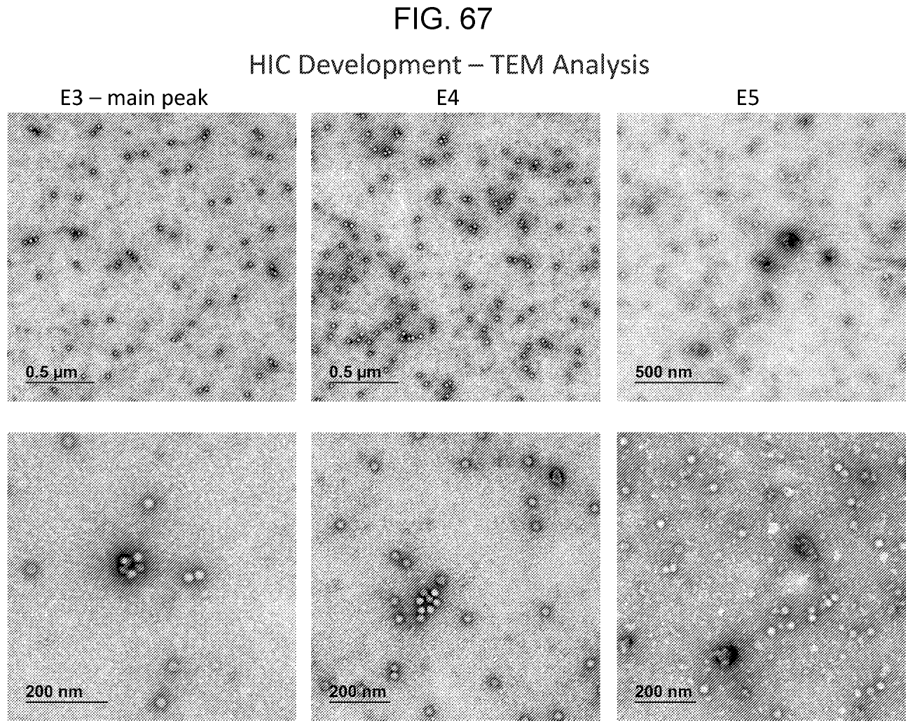

FIG. 65A shows an exemplary HIC chromatogram, with elution of rAAV particles in the E3, E4 and E5 fractions. Yield of total particles was highest in the E3 fraction, as can be seen by HPLC and ddPCR (FIG. 65B). FIG. 66B shows transmission electron micrographs of the rAAV particles from fractions E3, E4 and E5. In the main peak (E3) the rAAV vectors are evenly arranged, with the majority being full capsids. There are not many aggregates or damaged particles. The quality of the product, both in terms of proportion of full capsids, aggregates and damaged particles, decreased with each subsequent fraction.

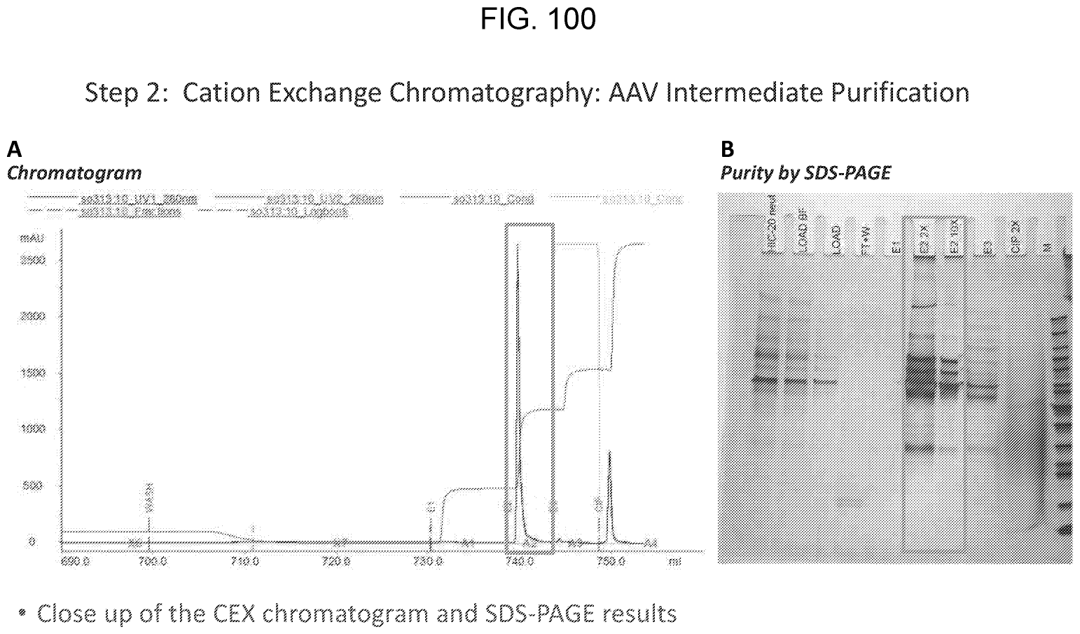

FIG. 100A shows an example chromatogram of rAAV particles purified from HIC eluate using CEX. Most rAAV particles elute in fraction E2, which is boxed. FIG. 100B shows an SDS-PAGE gel. Loaded, from left to right are: HIC-20 neut, LOAD-BF, LOAD, flow through+wash (FT+W), fractions E1, E2 diluted two-fold (E2.2×), E2 diluted ten-fold (E2.10×), E3, CIP, CIP.2× and a marker. rAAV particles are present in E2.2× and E2.10×, which are boxed.

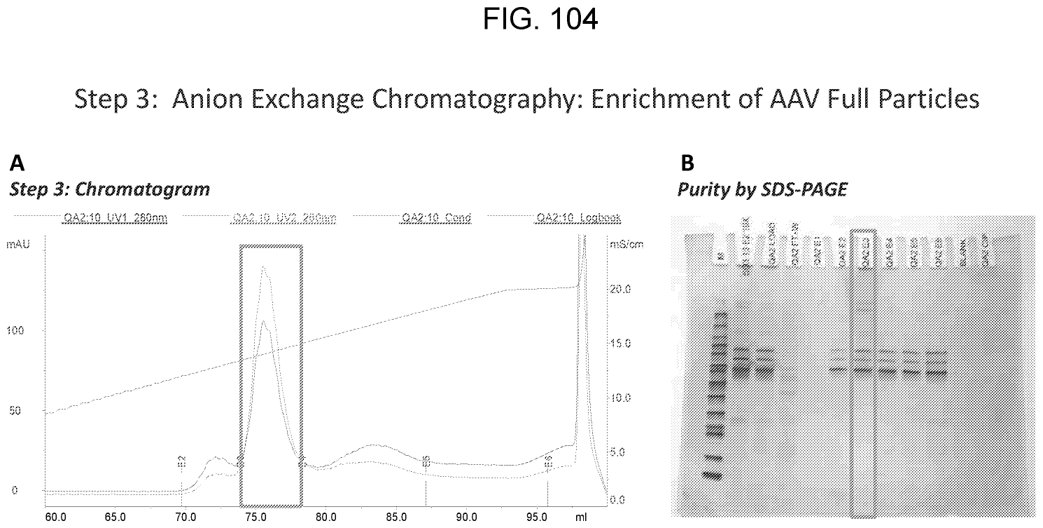

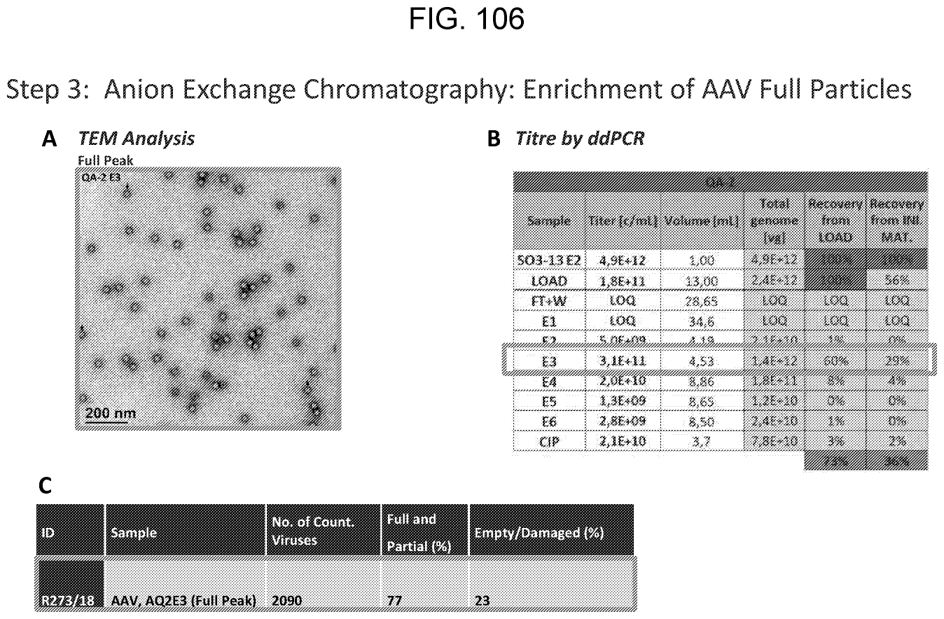

FIG. 104A shows an exemplary AEX chromatogram of the further purification of the CEX eluate. Full rAAV particles are enriched in the E3 fraction, which is boxed. Full particle enrichment is achieved by separation of full and empty particles based on the charge of the particles. Full particles are very slightly more negatively charged than empty particles due to the presence of the DNA genome. Separation can be further enhanced by removal of MgCl₂ from the buffers for serotype AAV8 particles. FIG. 104B shows purity by SDS-PAGE gel. Lanes show, from left to right: a marker, SQ3 13 E2 10×, QA2 LOAD, QA2 FT+W, fractions QA2 E1, QA2 E2, QA2 E3, QA2 E4, QA2 E5, QA2 E6, BLANK and QA2 CIP. Transmission electron microgram of fraction QA2 E3 from the chromatogram of FIG. 104A shows the recovery of AAV particles in the E3 fraction (FIG. 106A). When 2090 full and empty particles were counted, 77% were full, and 23% were empty or damaged (FIG. 106C). When the titre was determined by droplet digital PCR (ddPCR), fraction E3 had a titer of 3.1×10\hat{ }11 c/mL, a volume of 4.53 mL and 1.4×10\hat{ }12 vector genomes. Recovery from the input sample loaded on the column was 60%, and recovery from initial starting material was 29% (FIG. 106B).

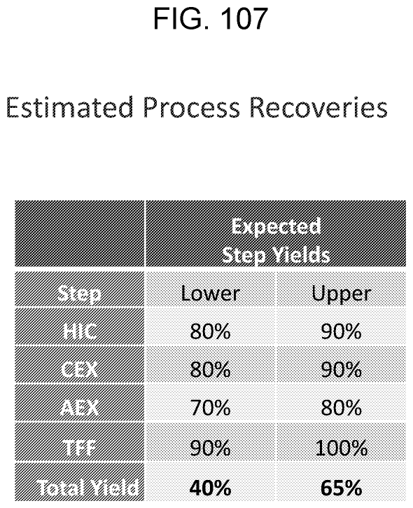

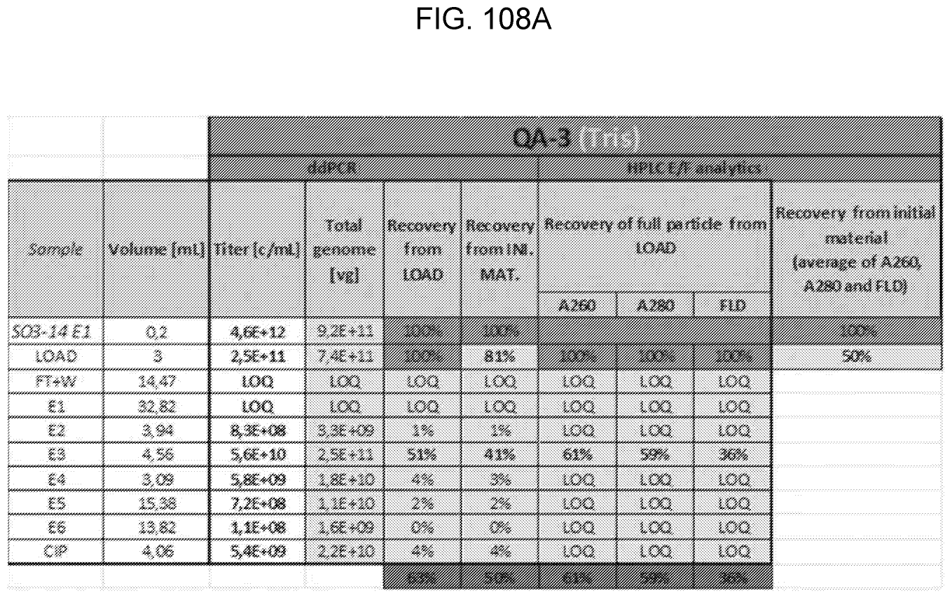

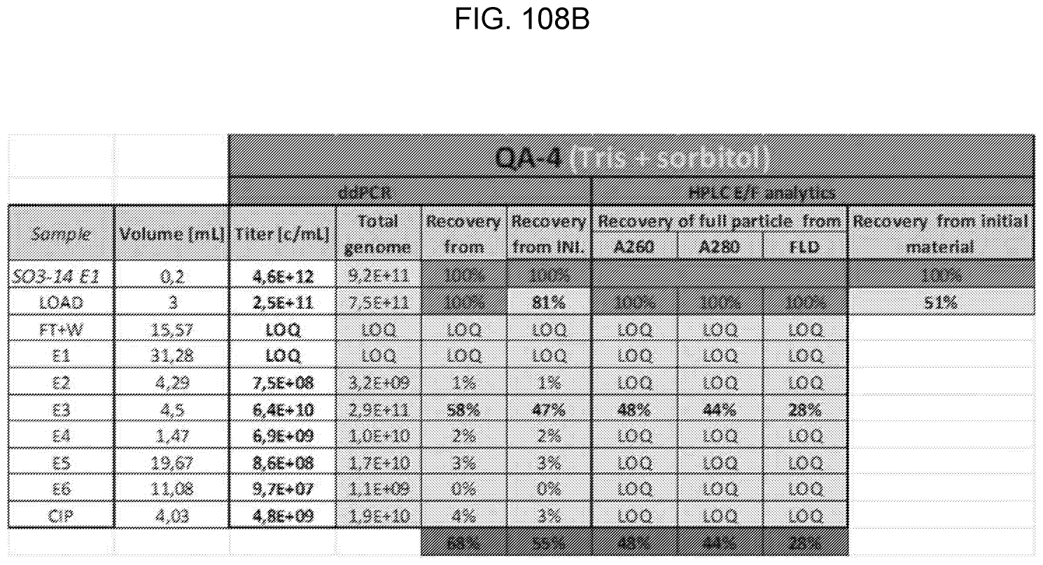

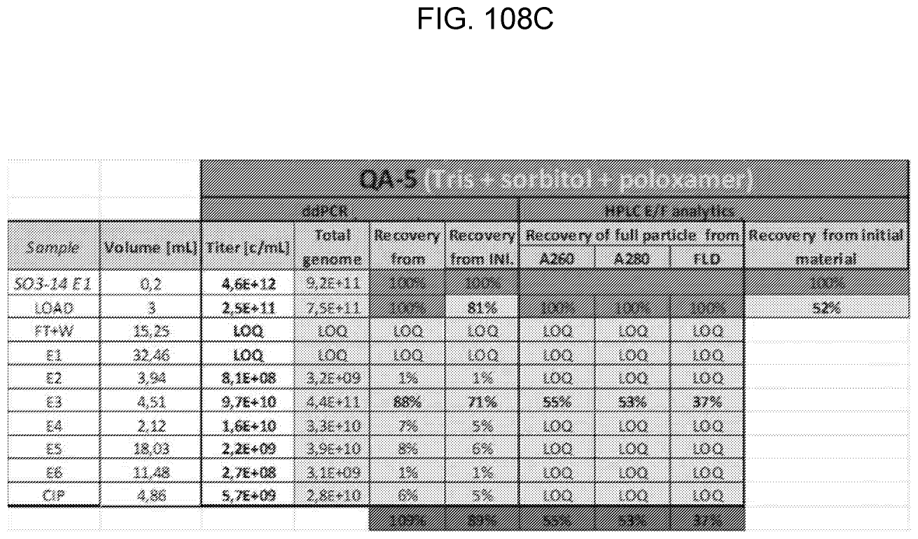

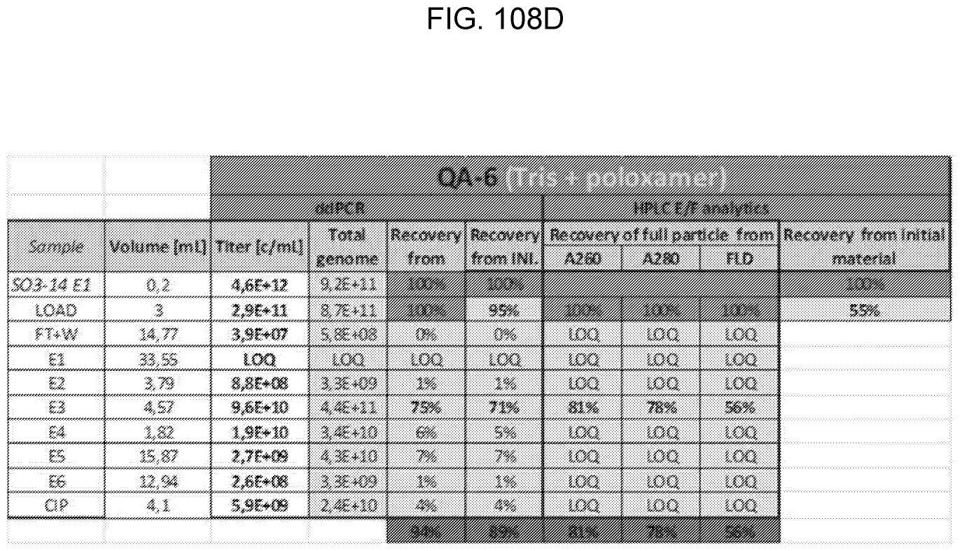

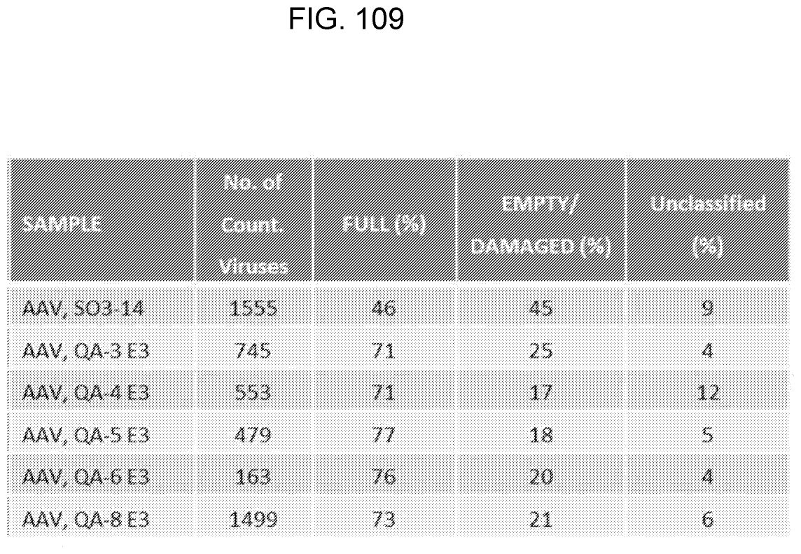

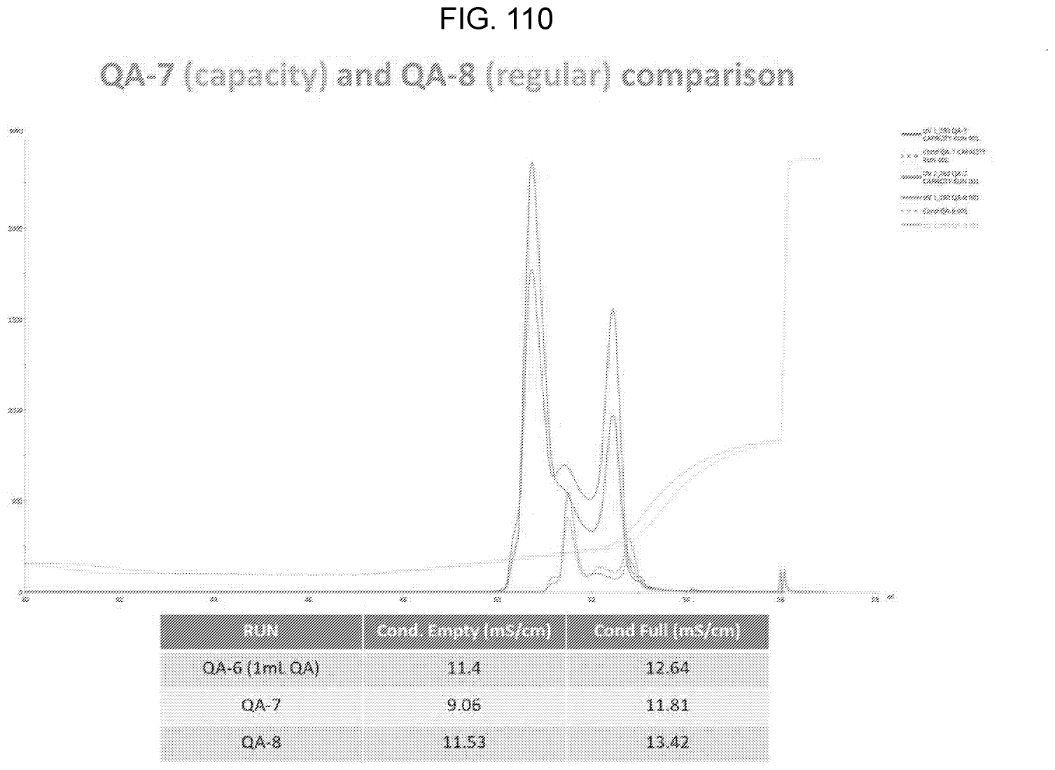

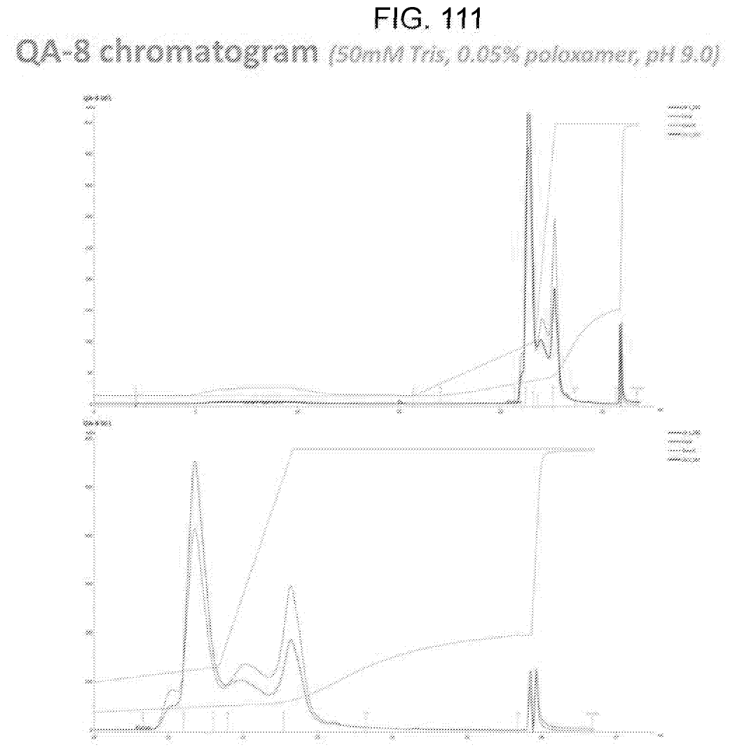

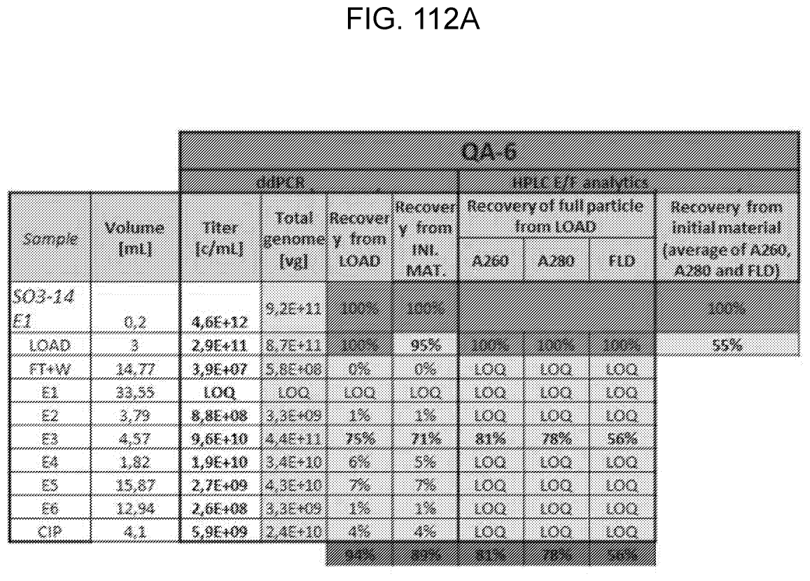

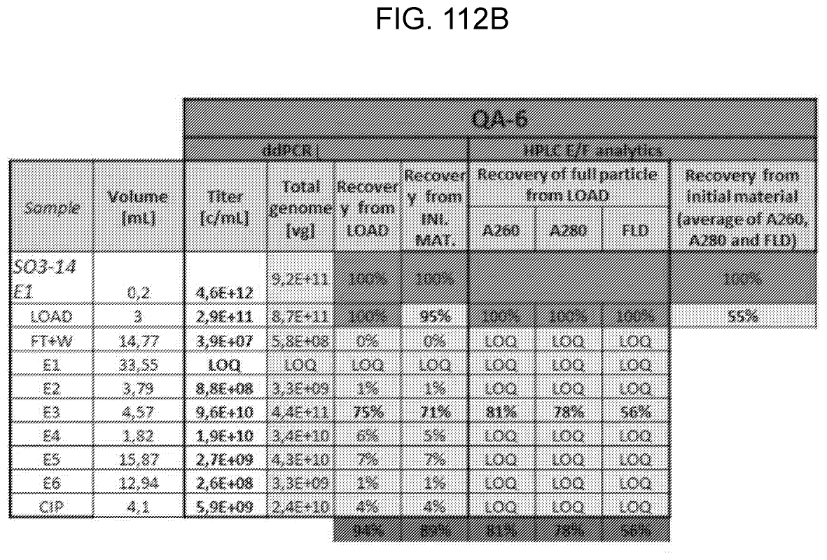

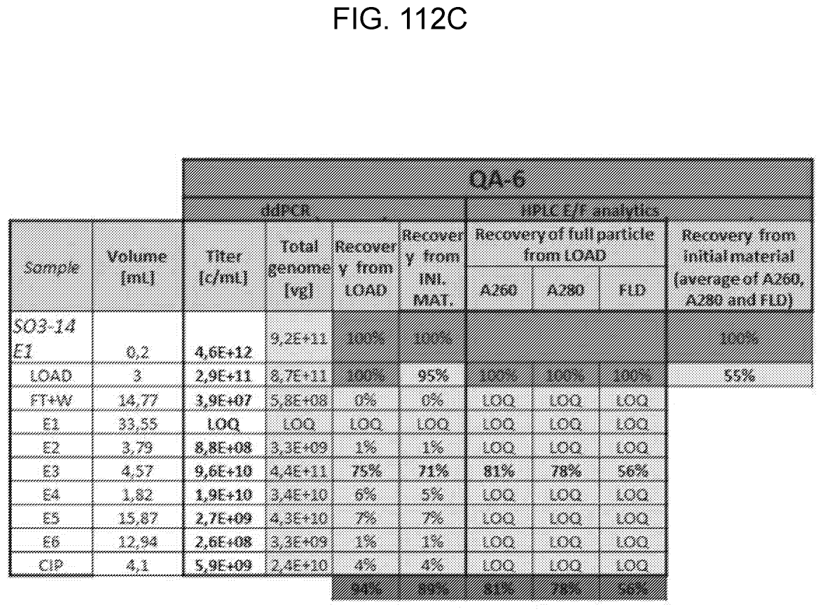

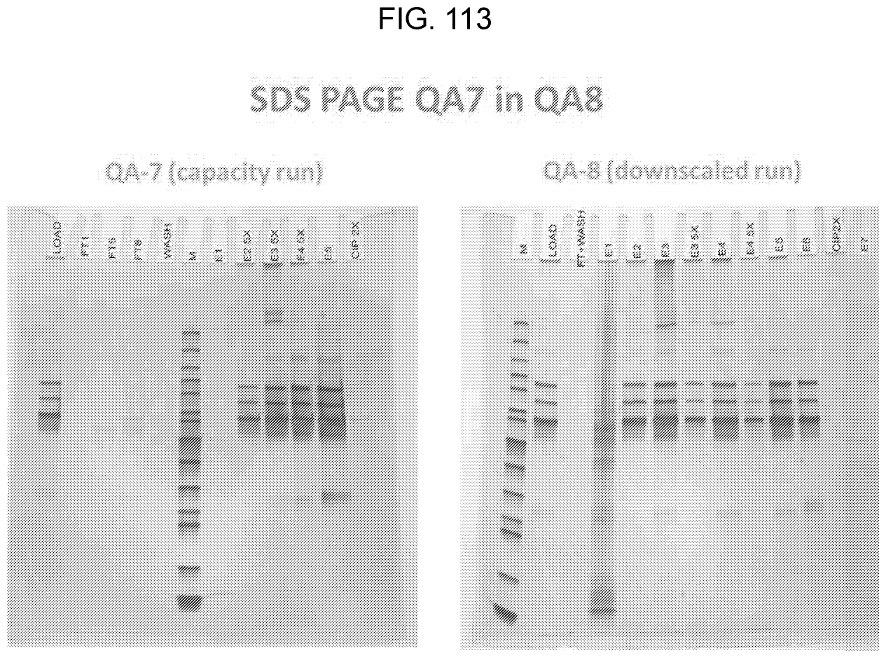

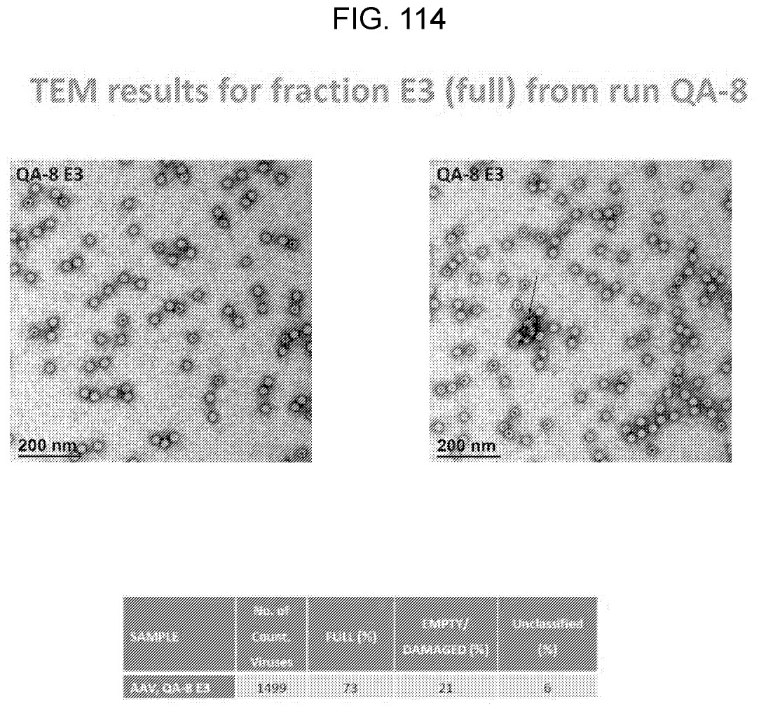

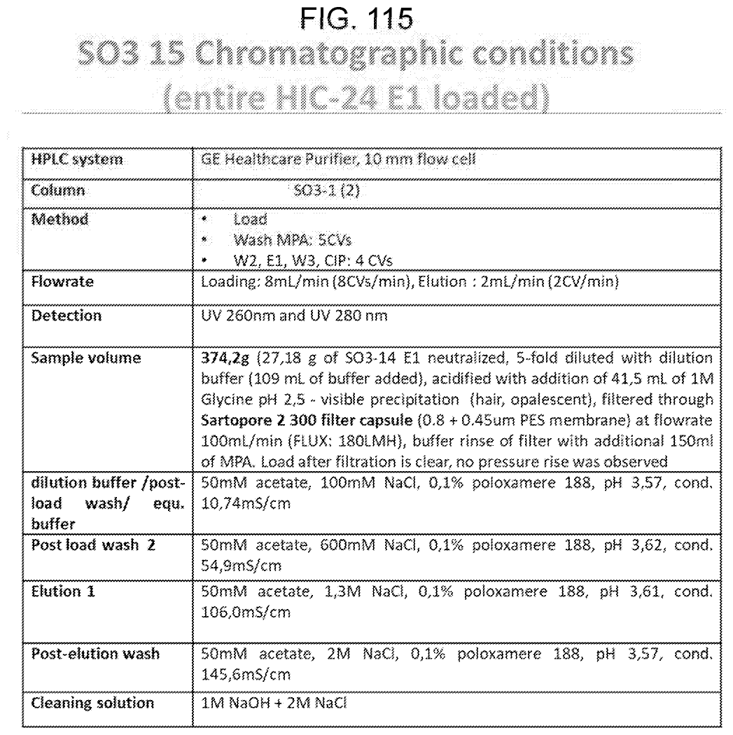

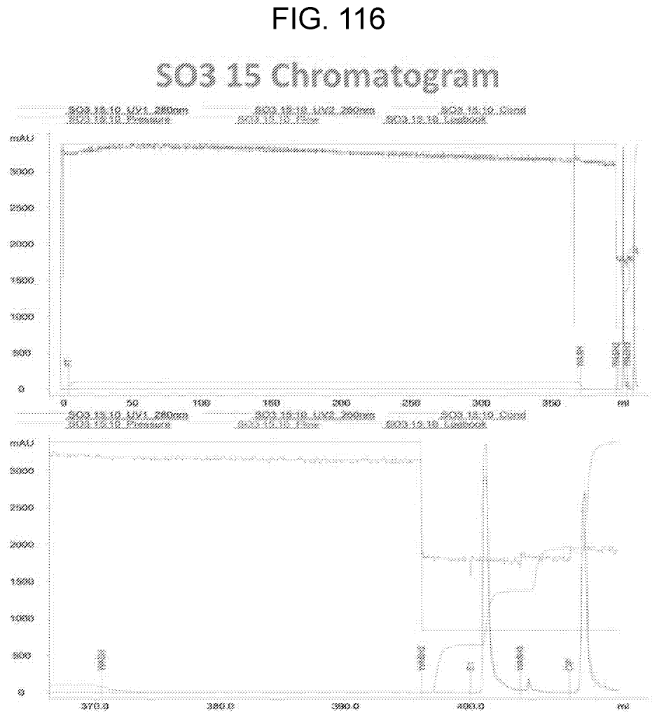

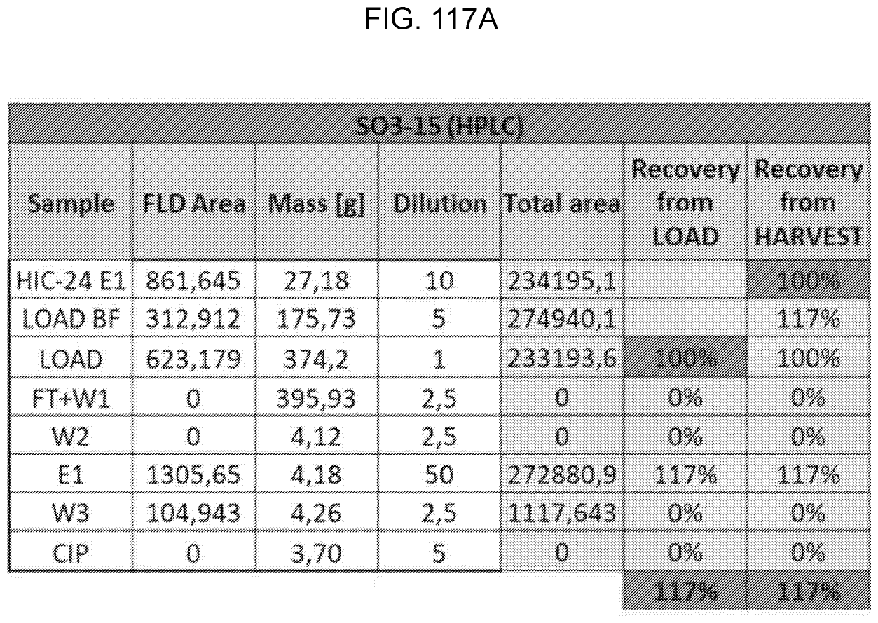

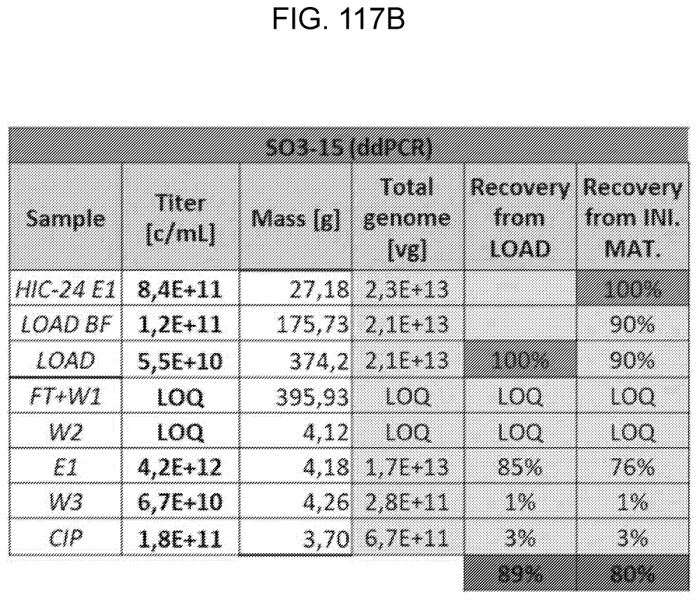

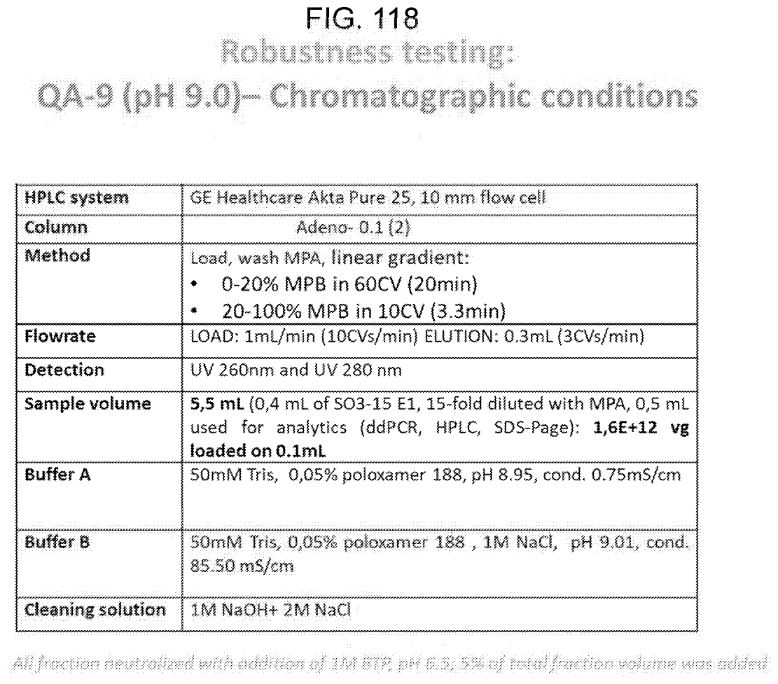

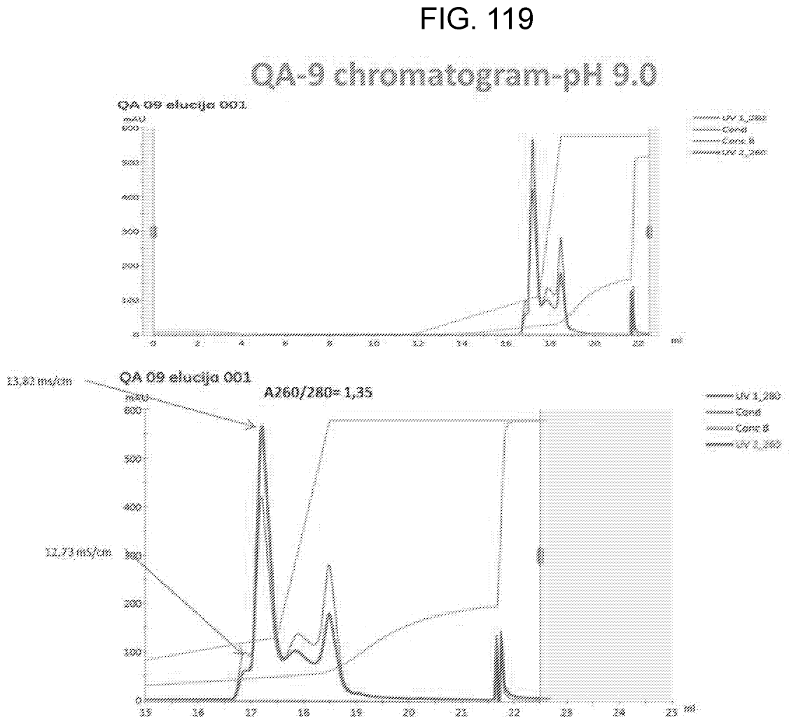

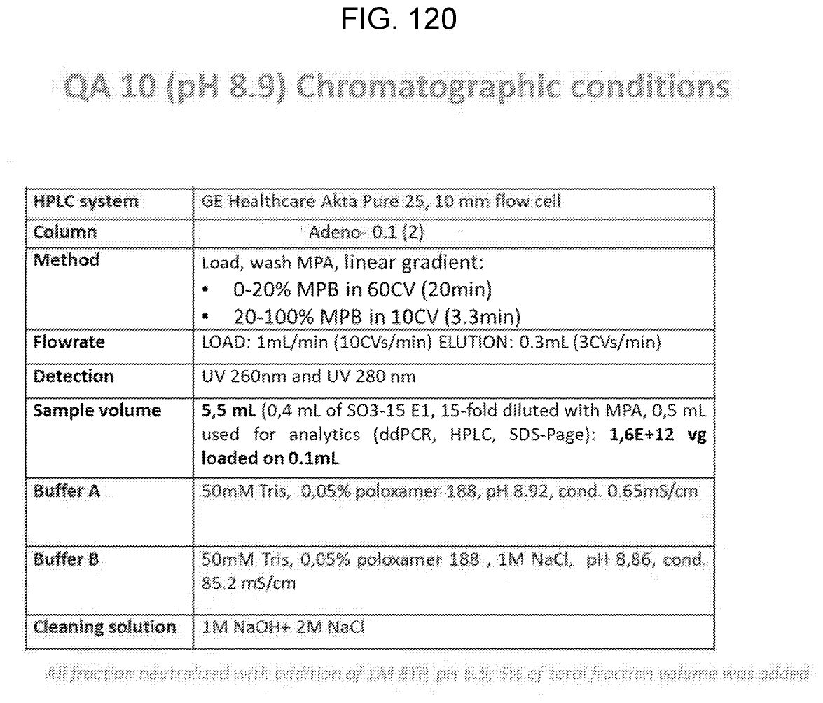

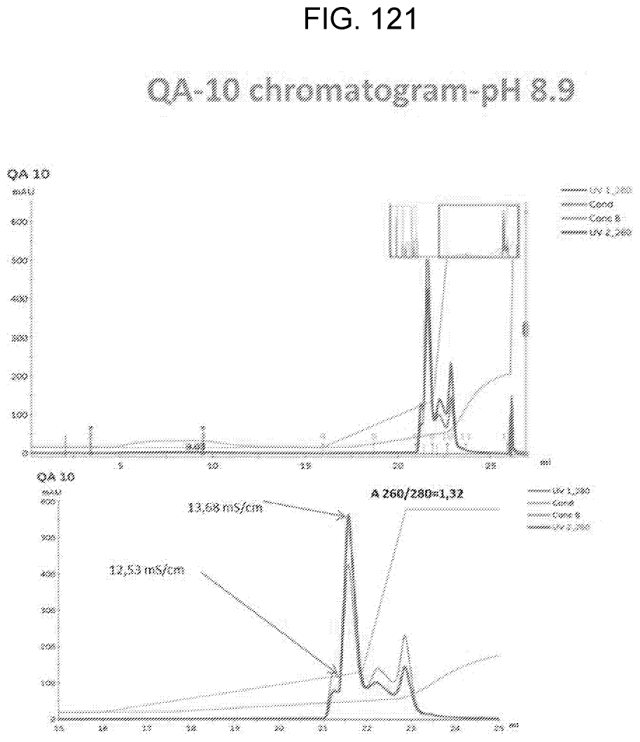

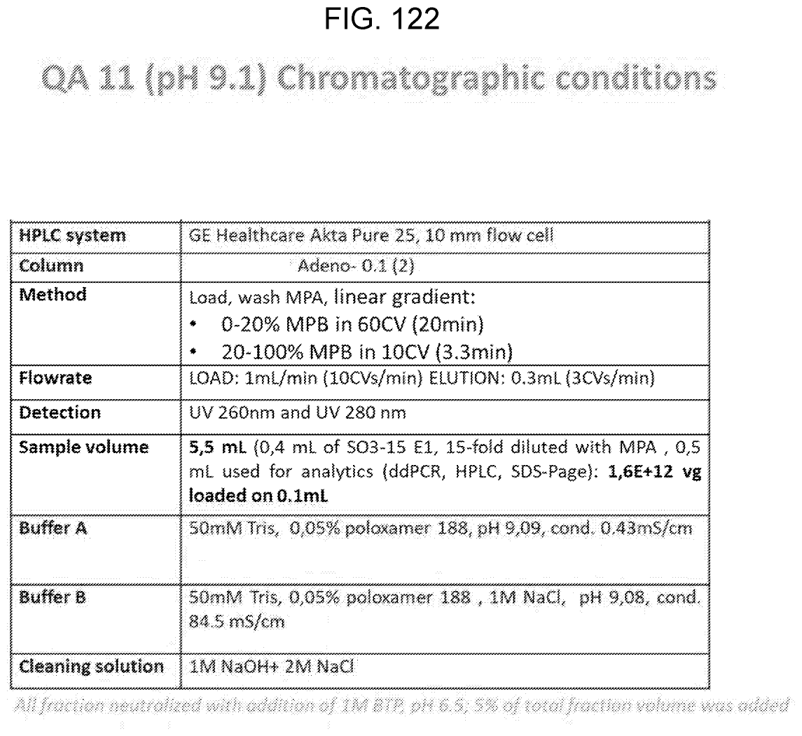

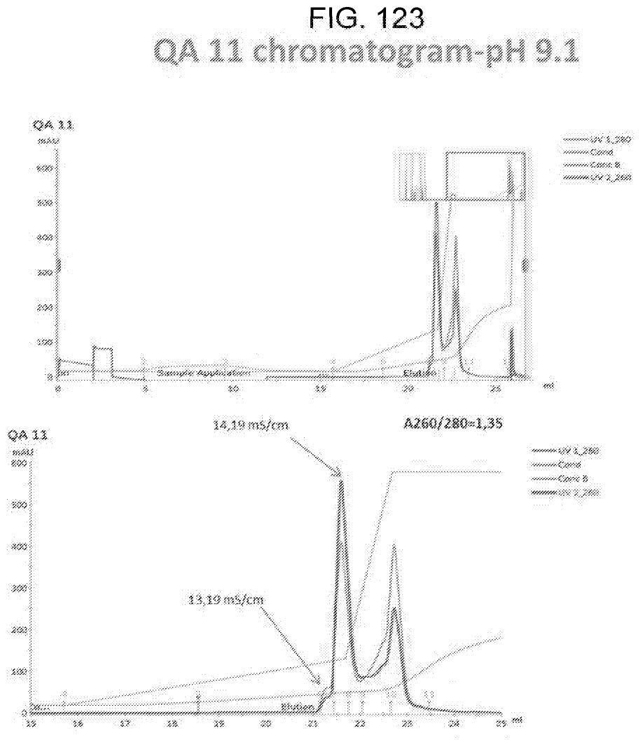

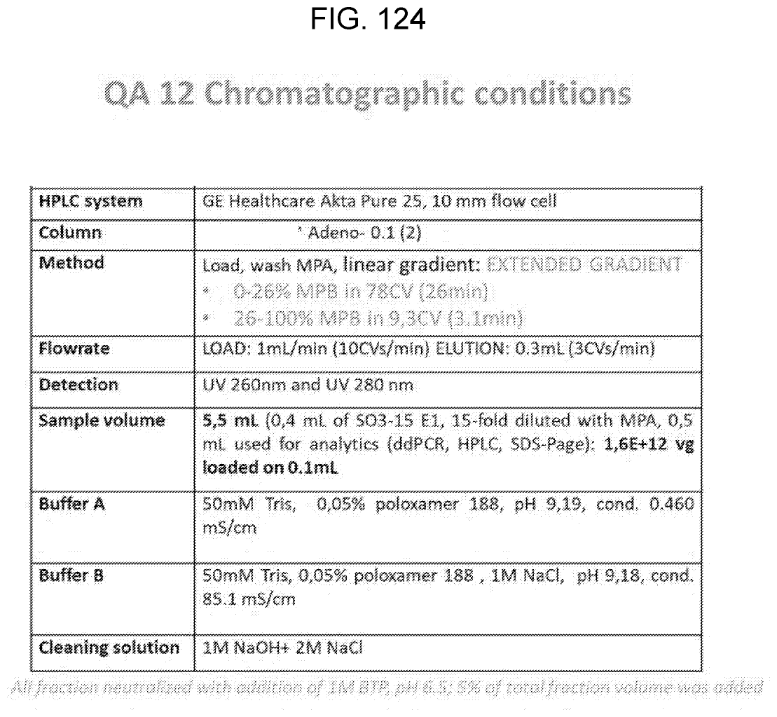

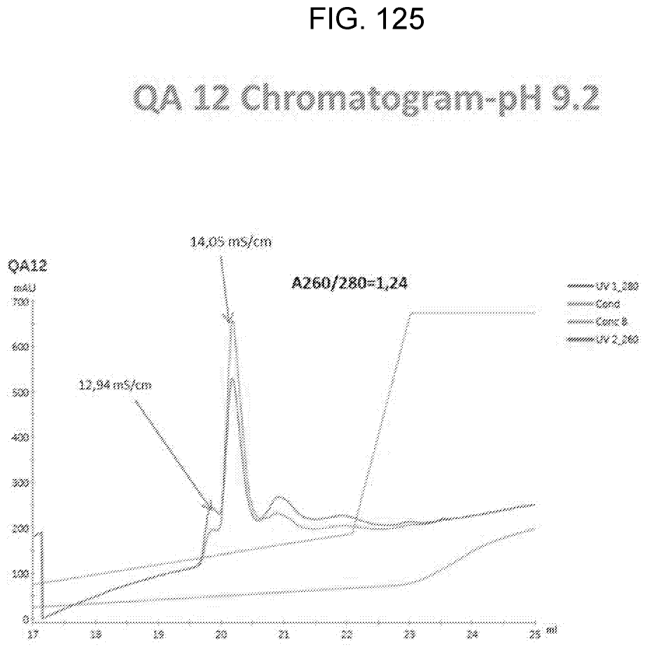

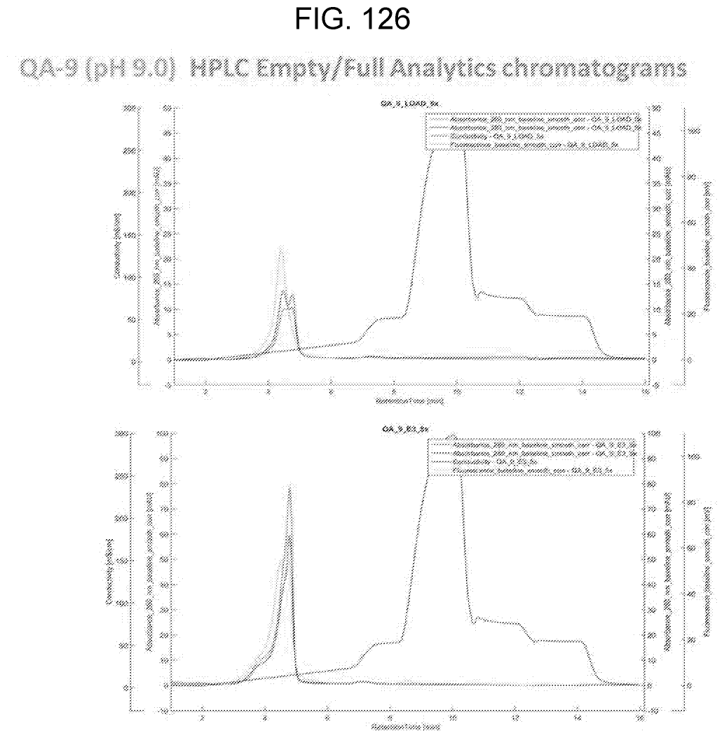

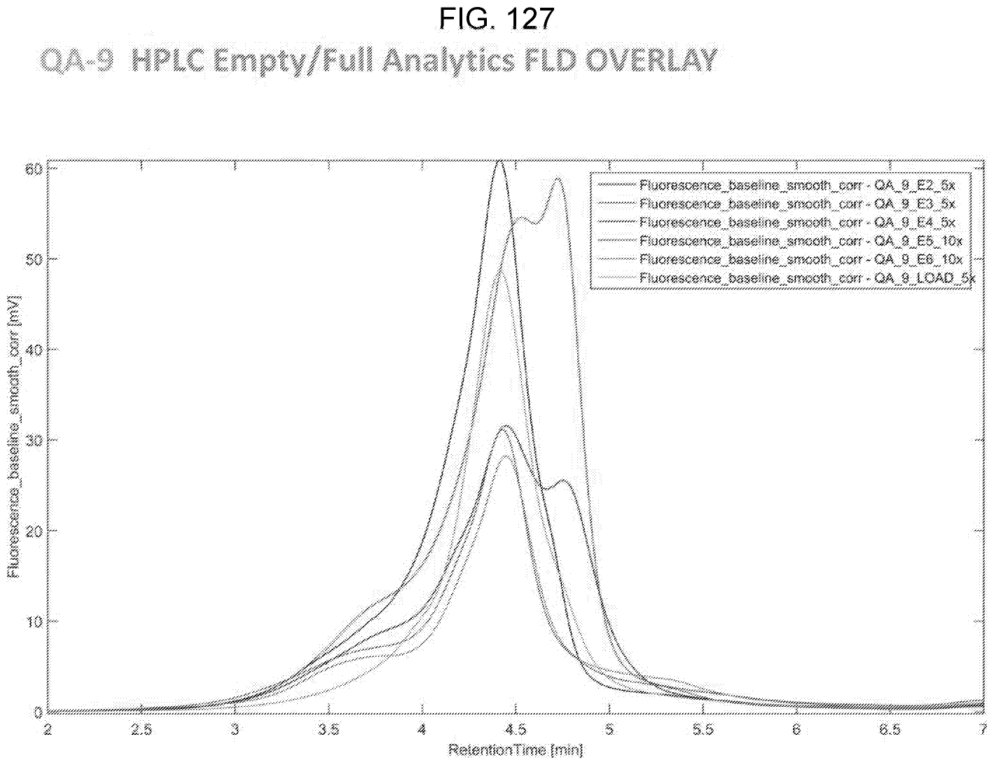

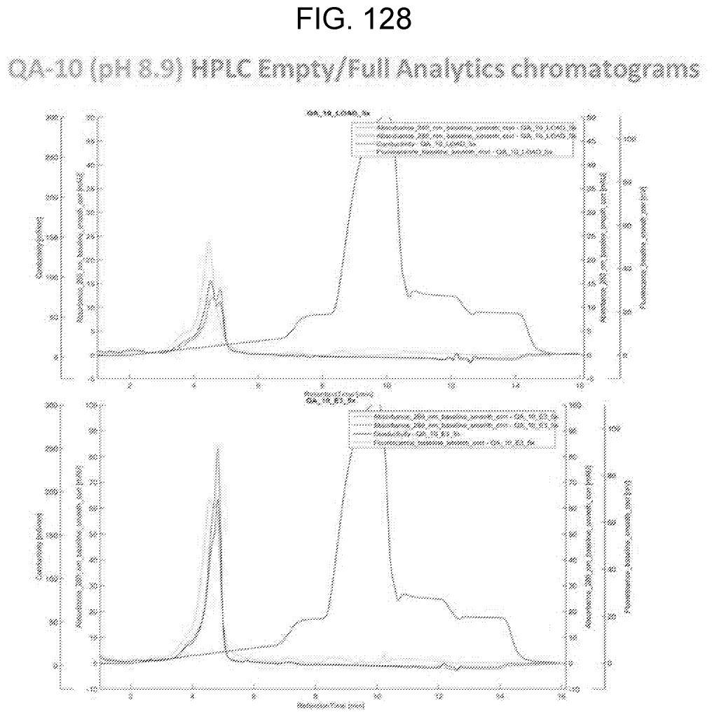

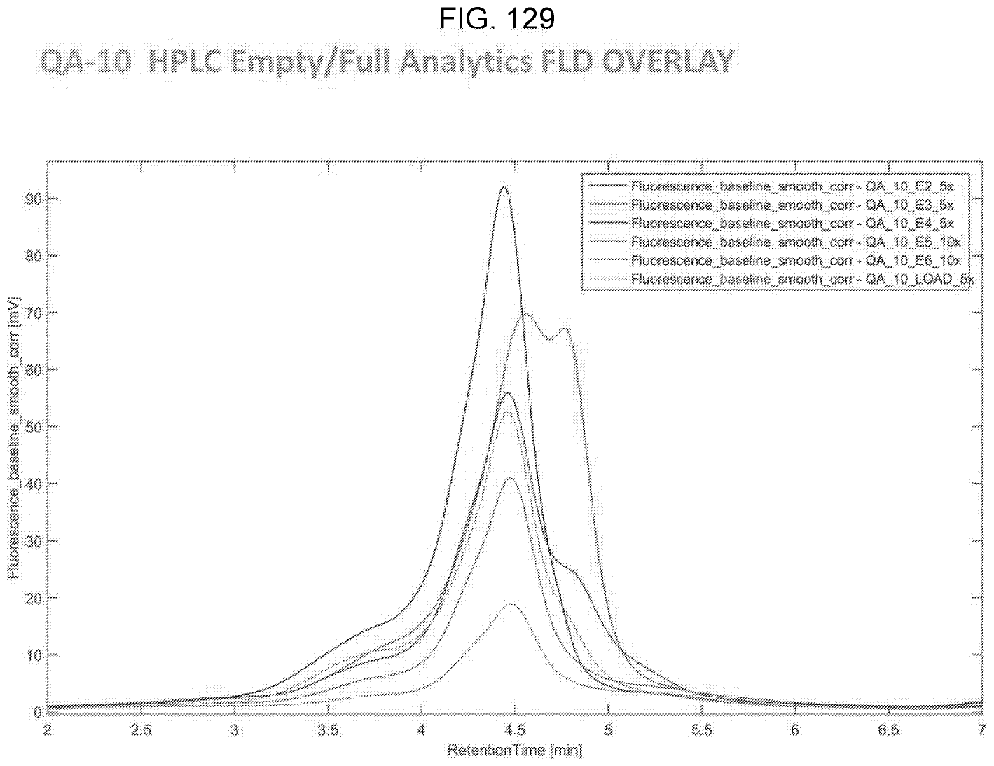

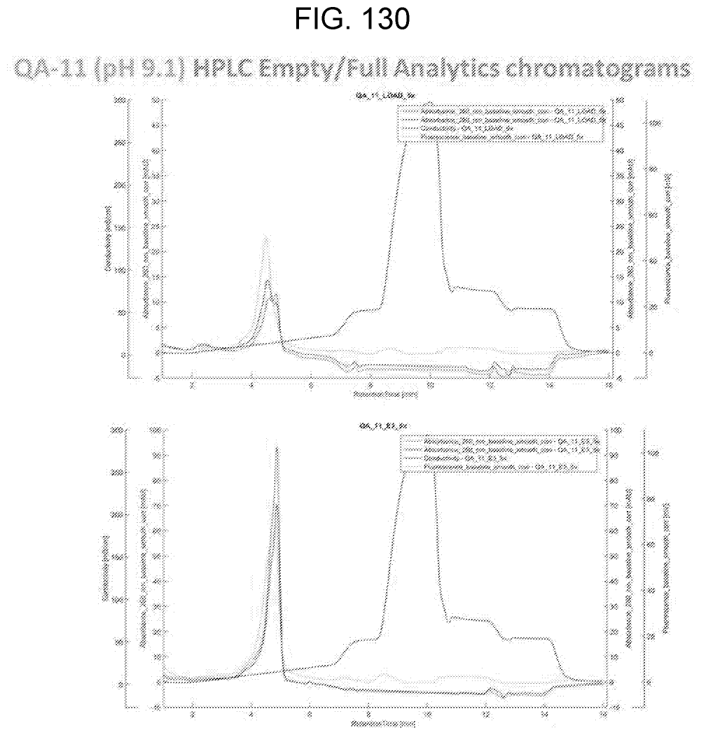

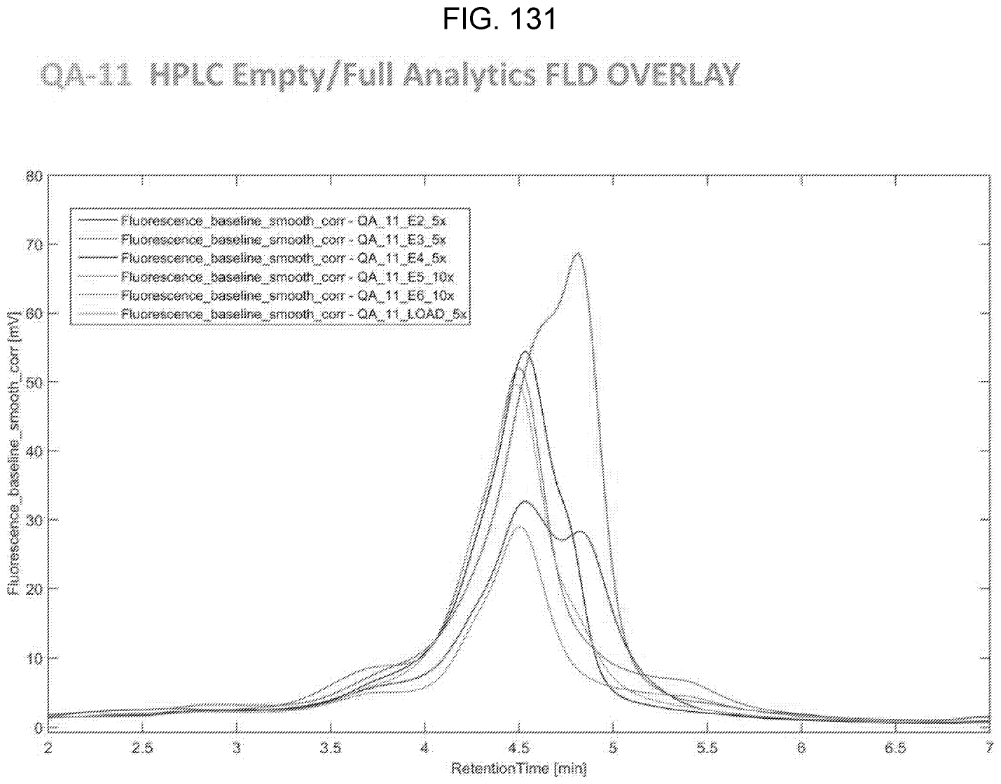

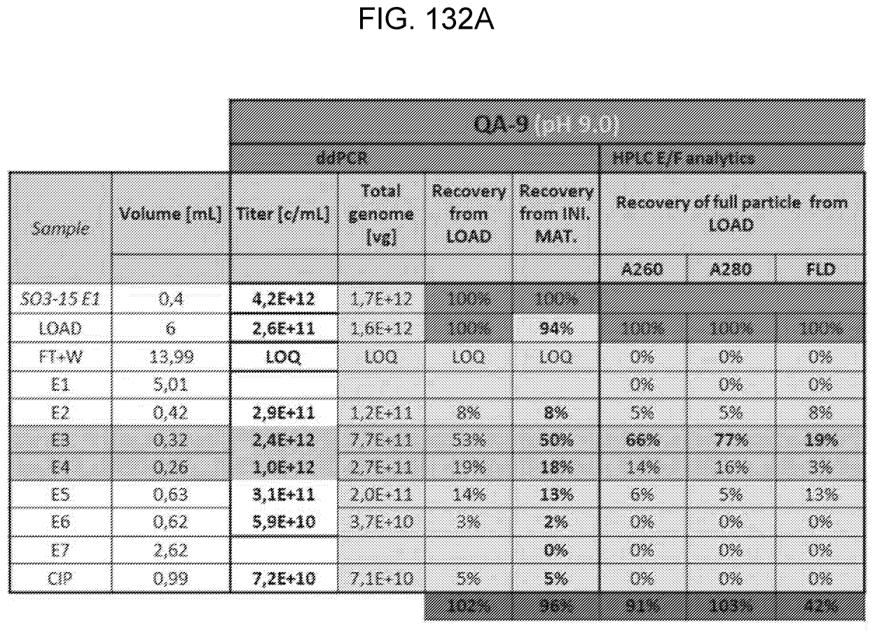

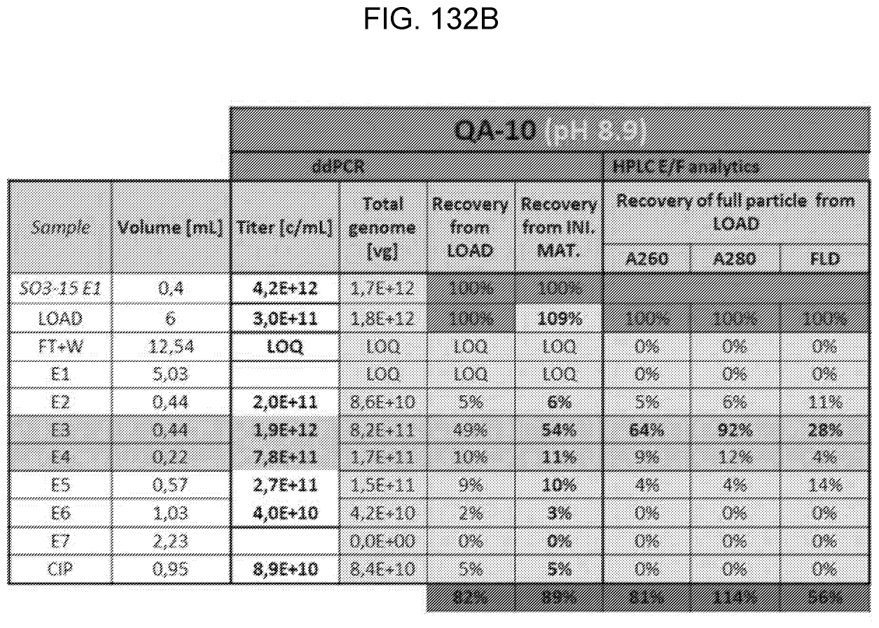

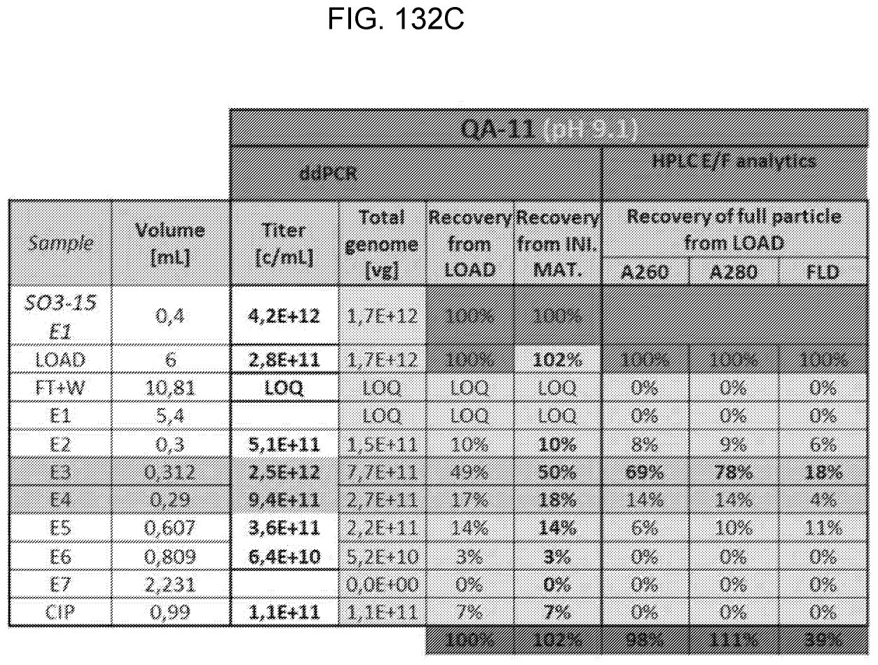

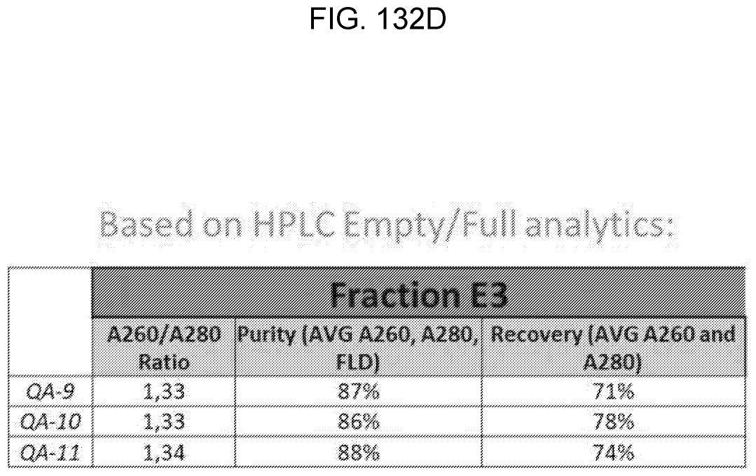

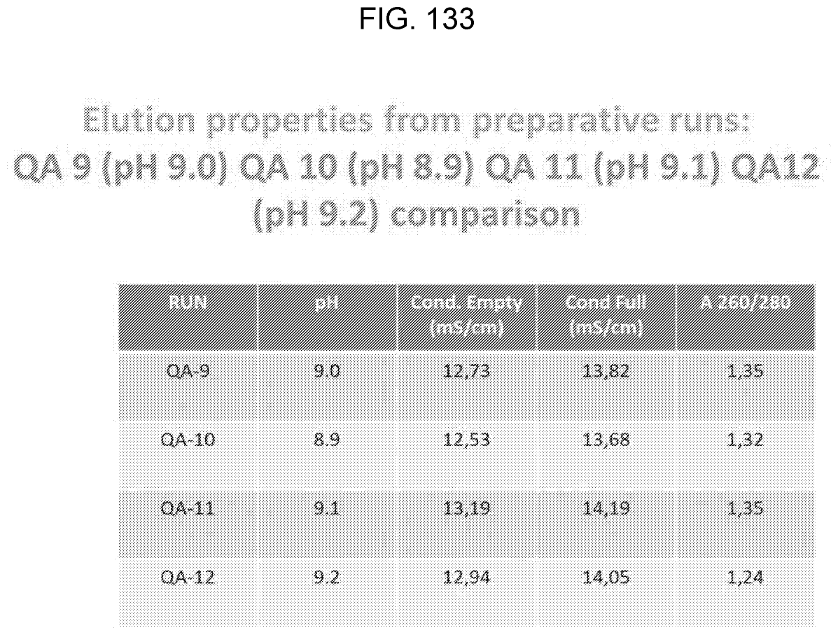

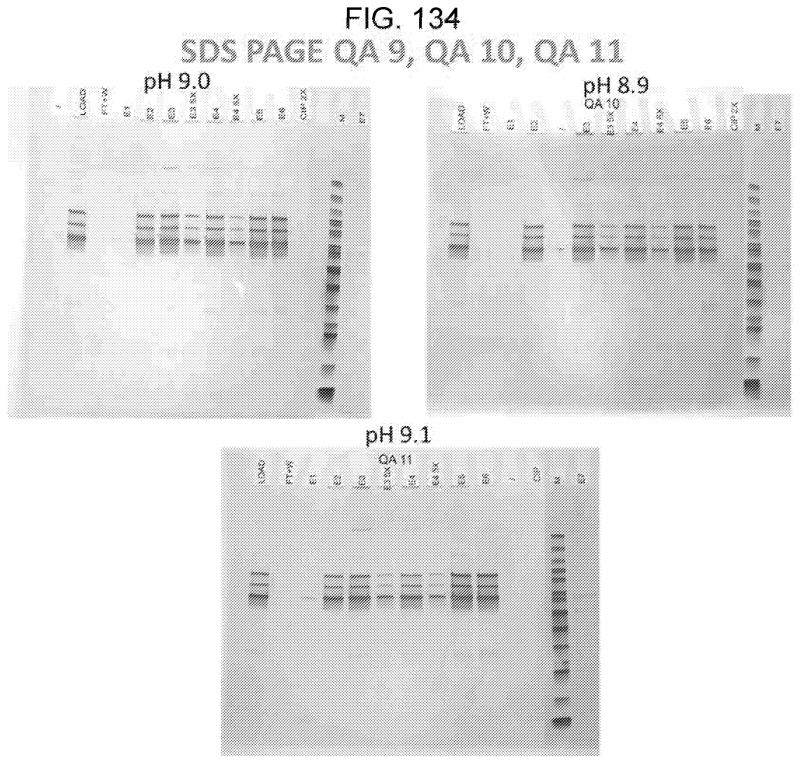

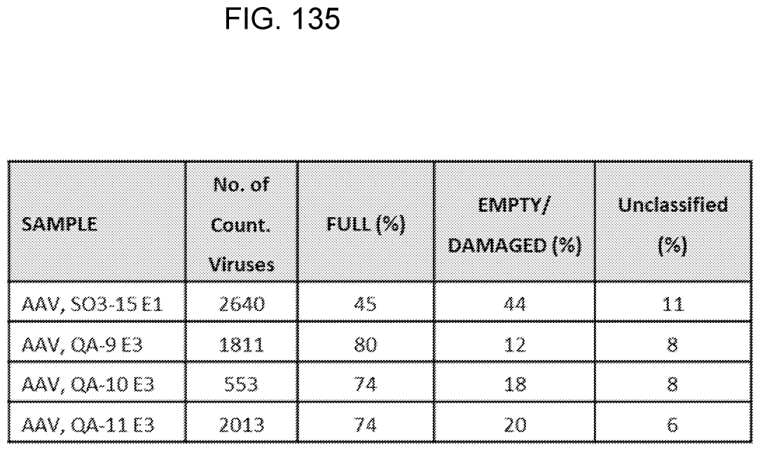

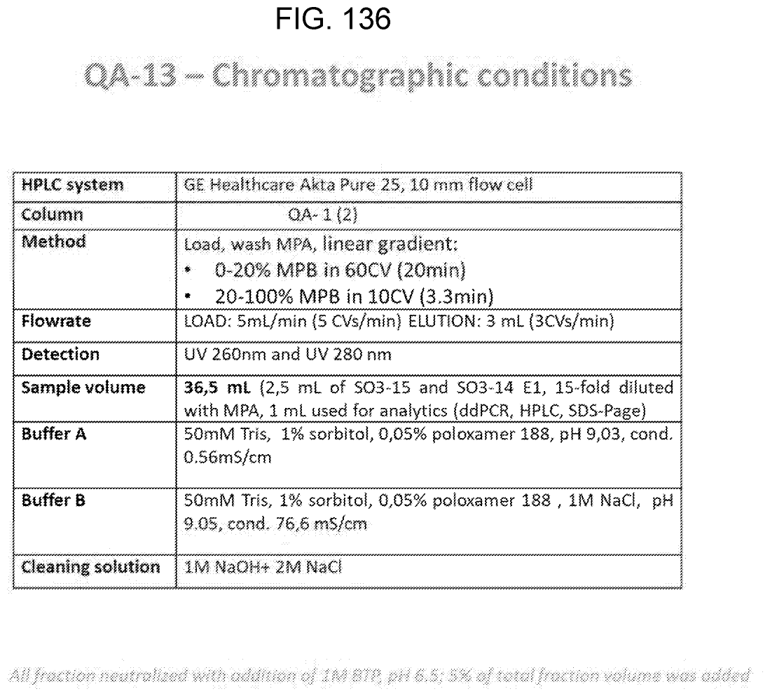

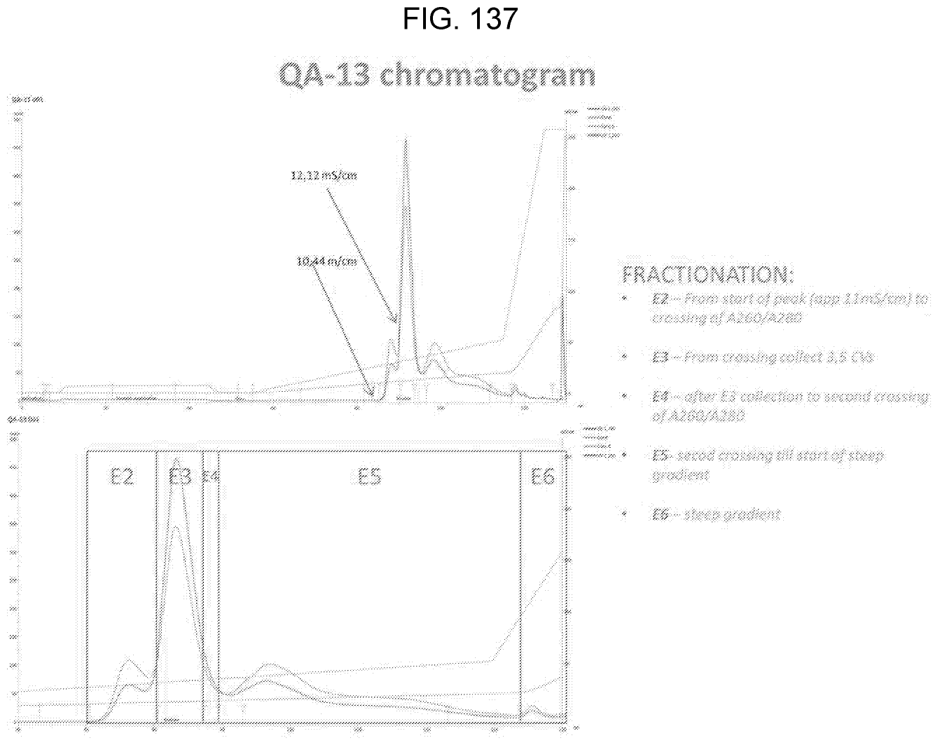

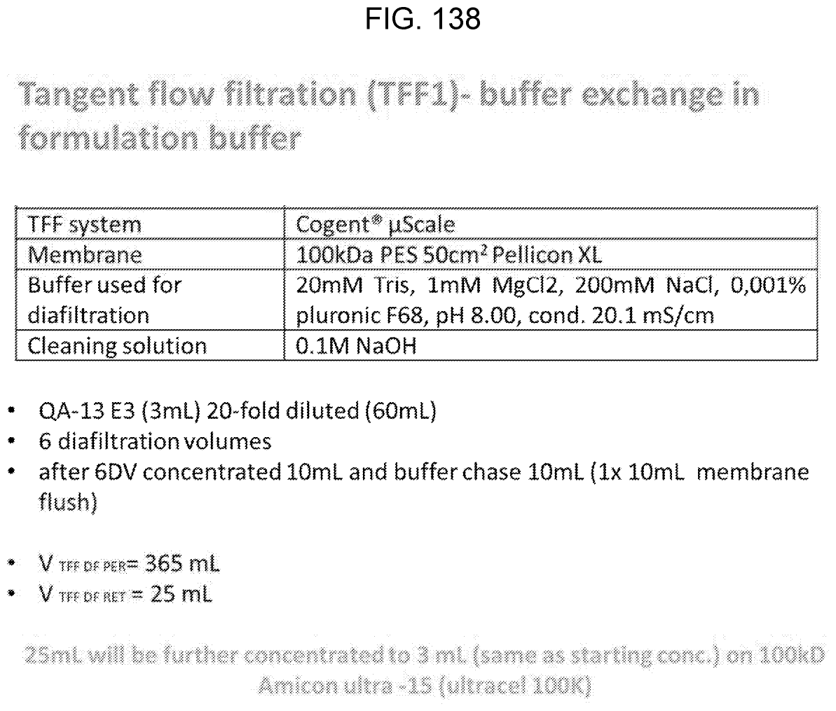

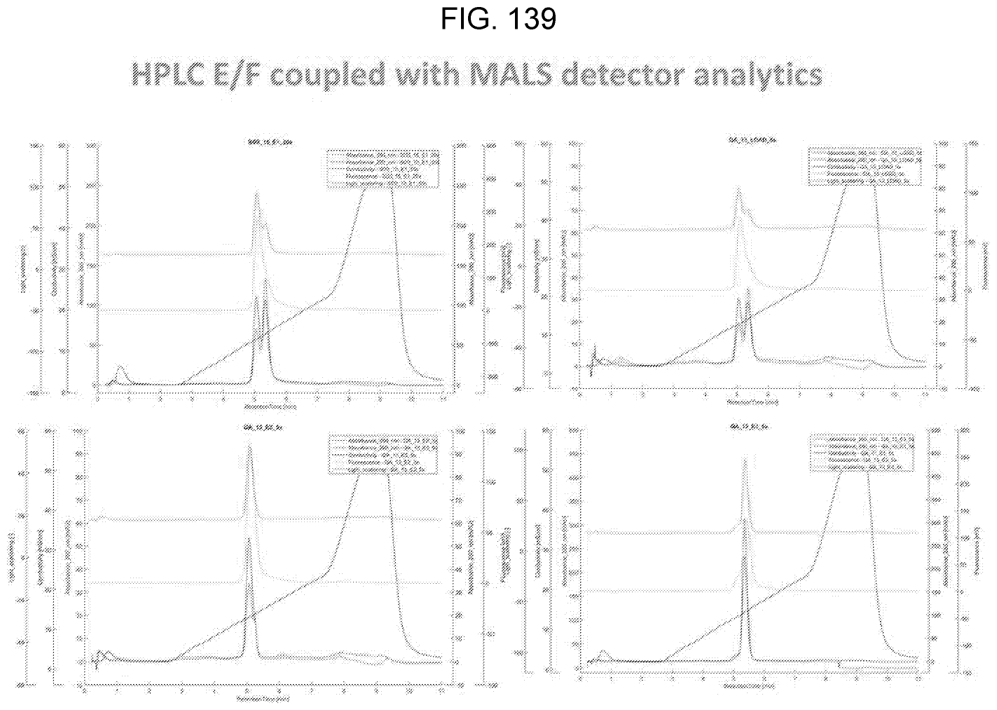

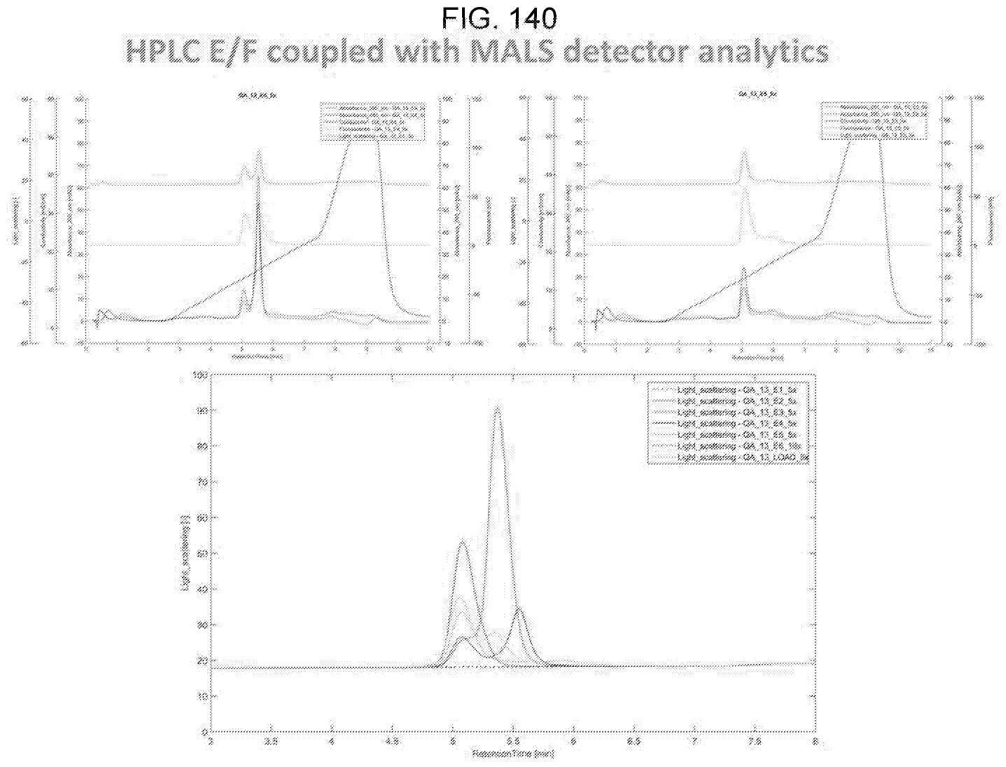

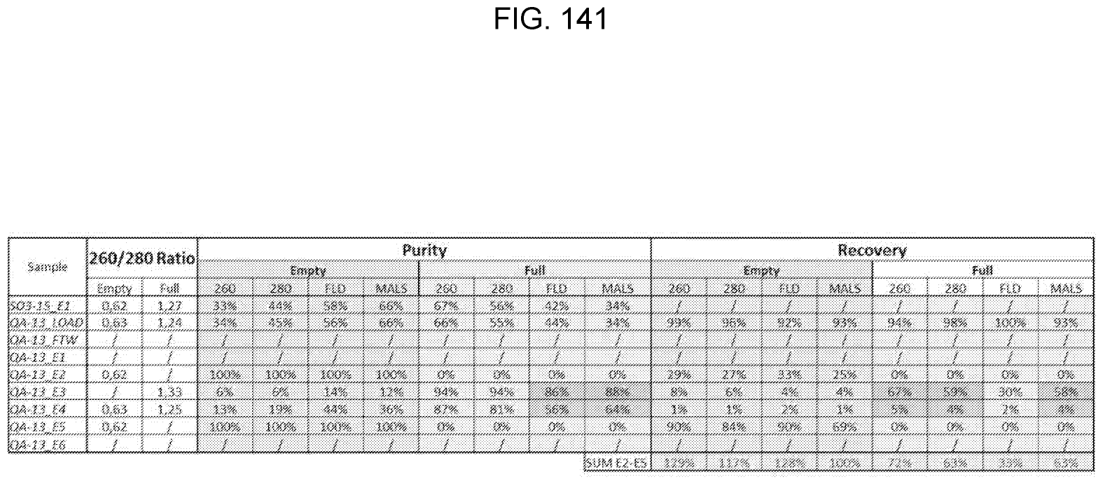

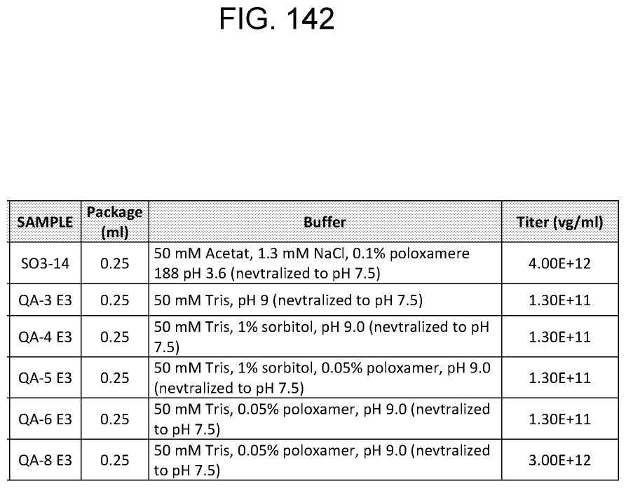

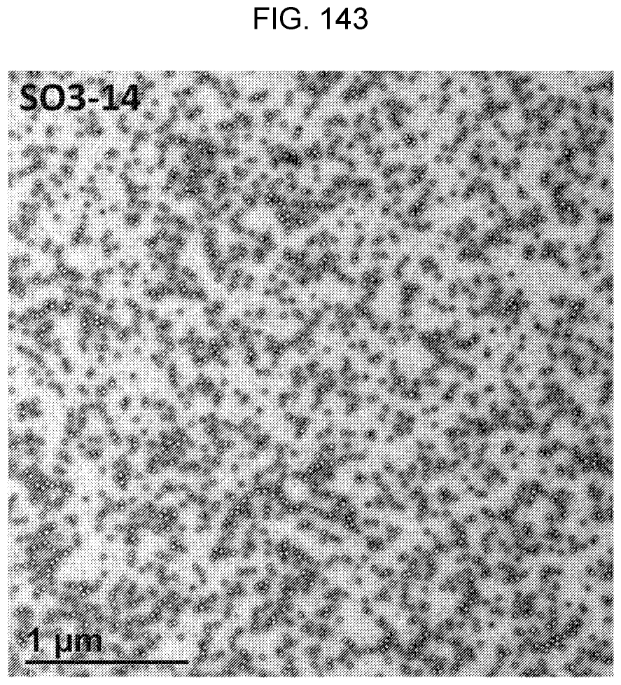

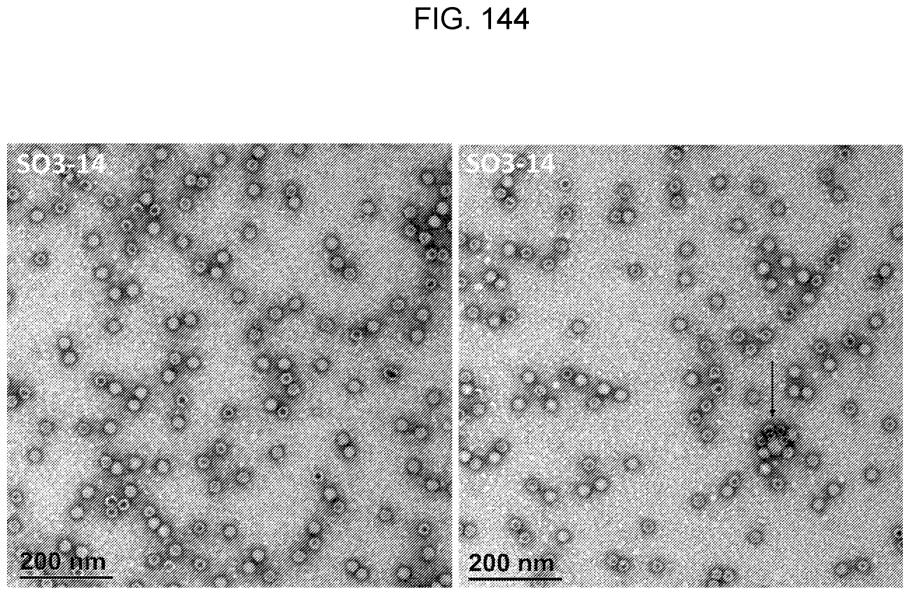

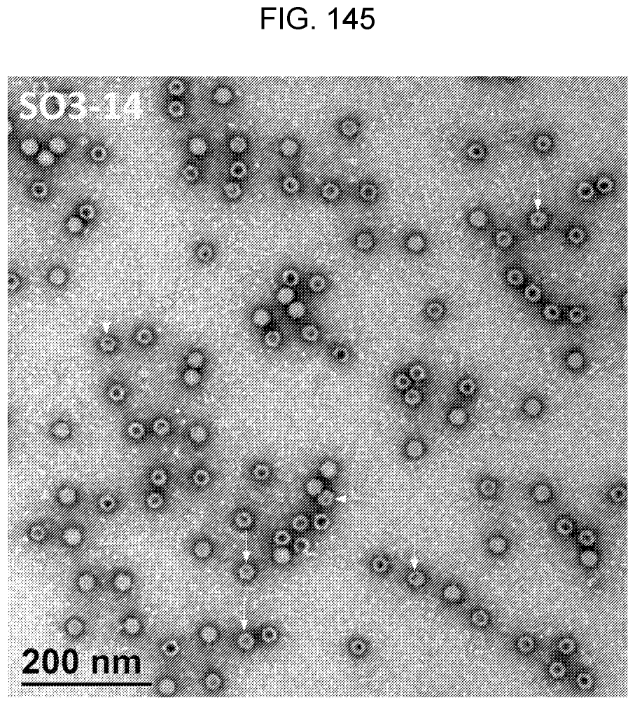

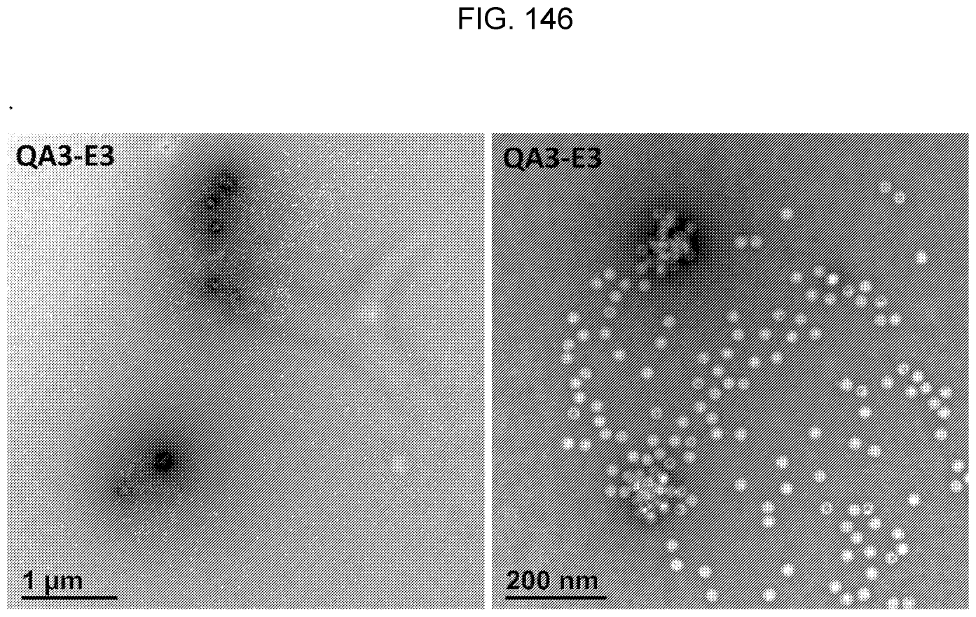

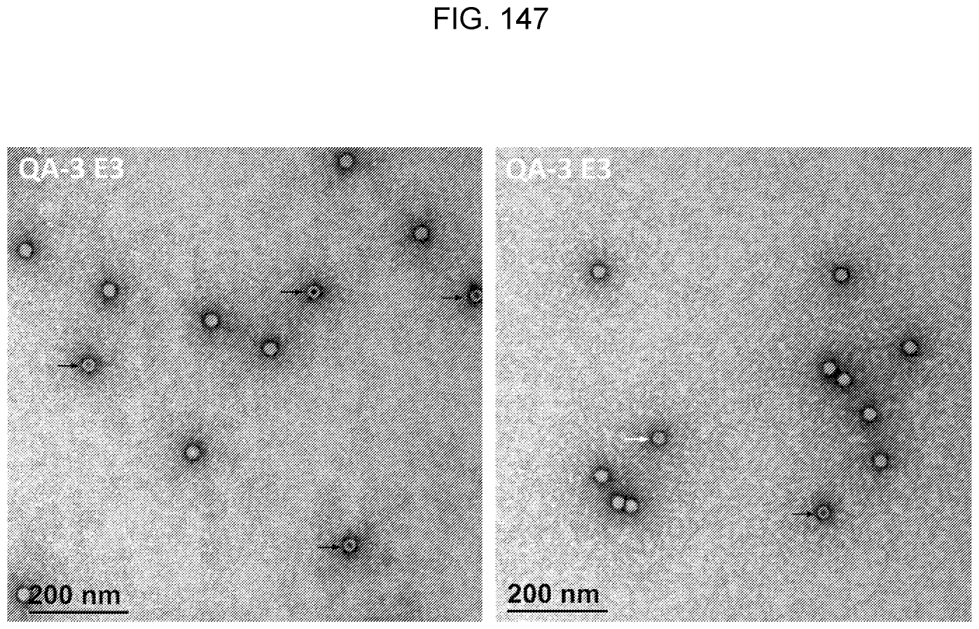

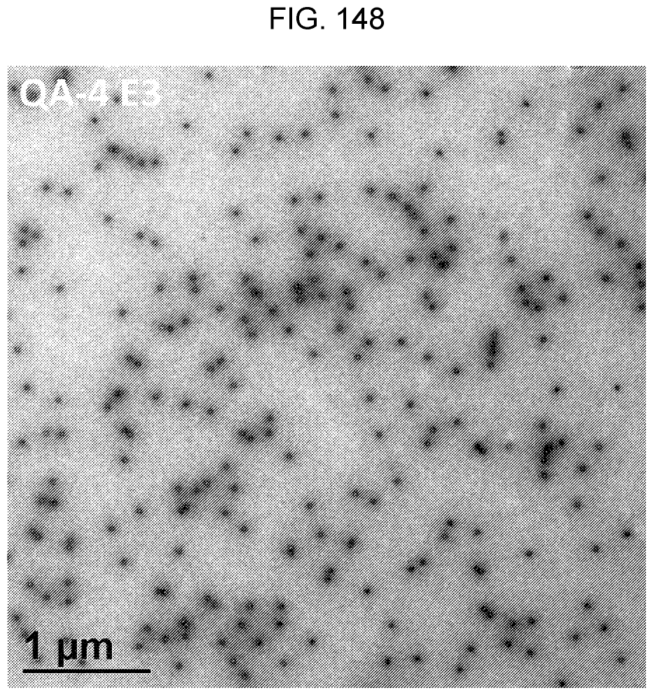

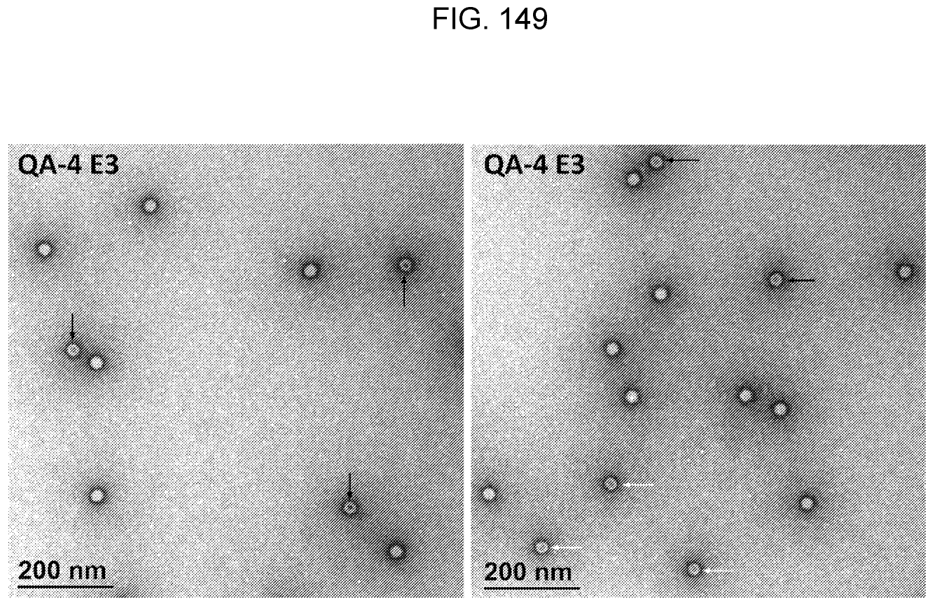

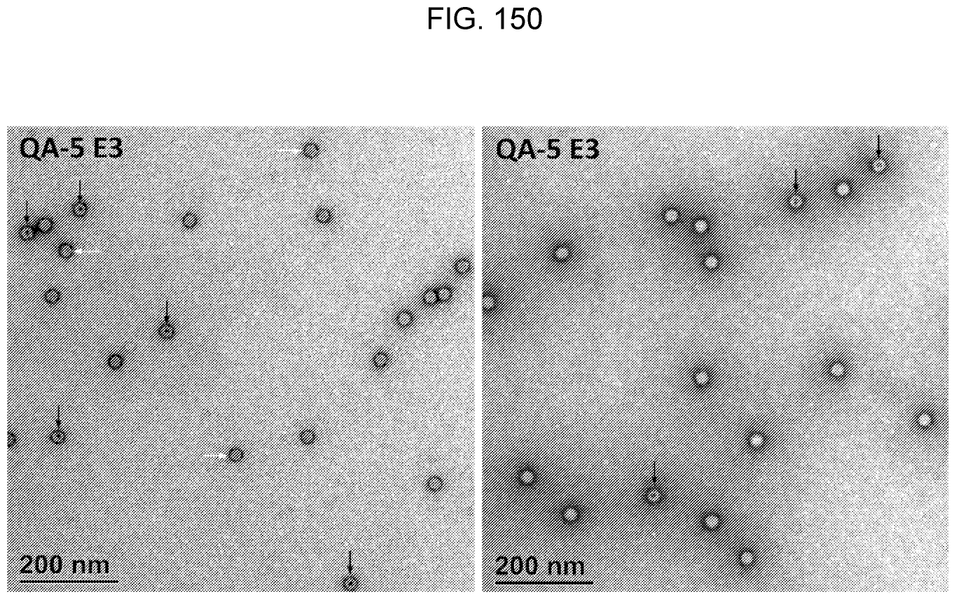

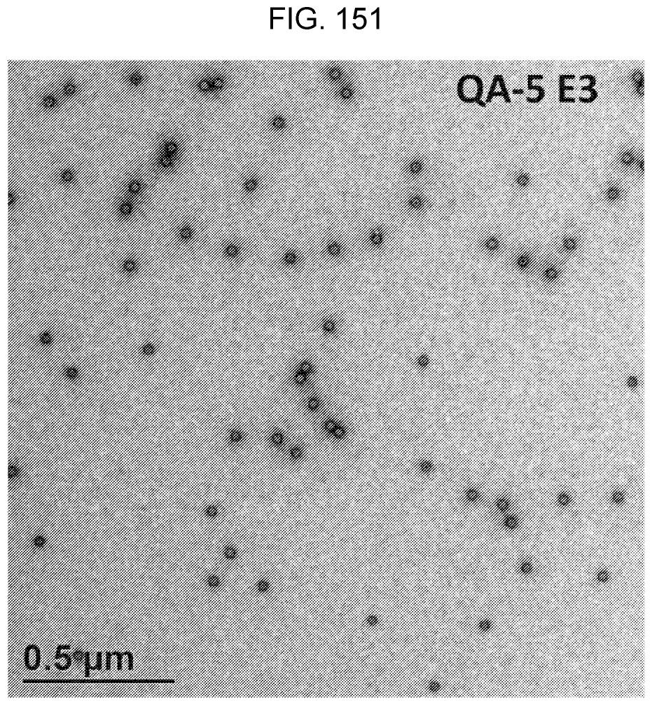

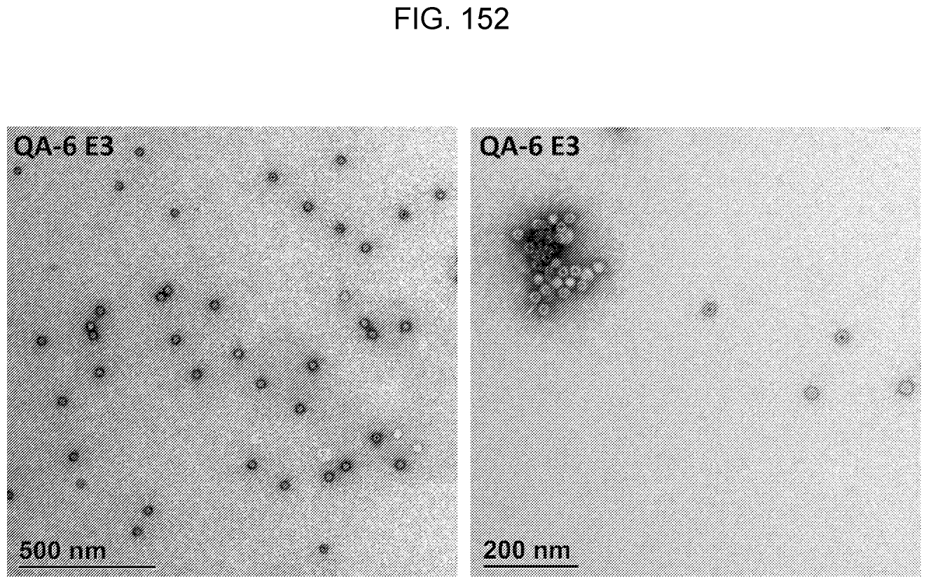

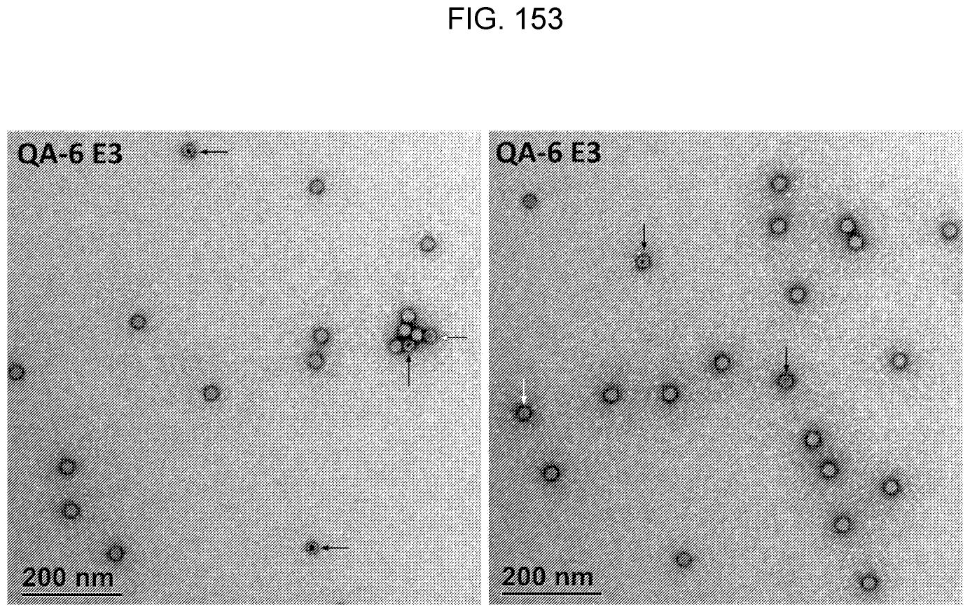

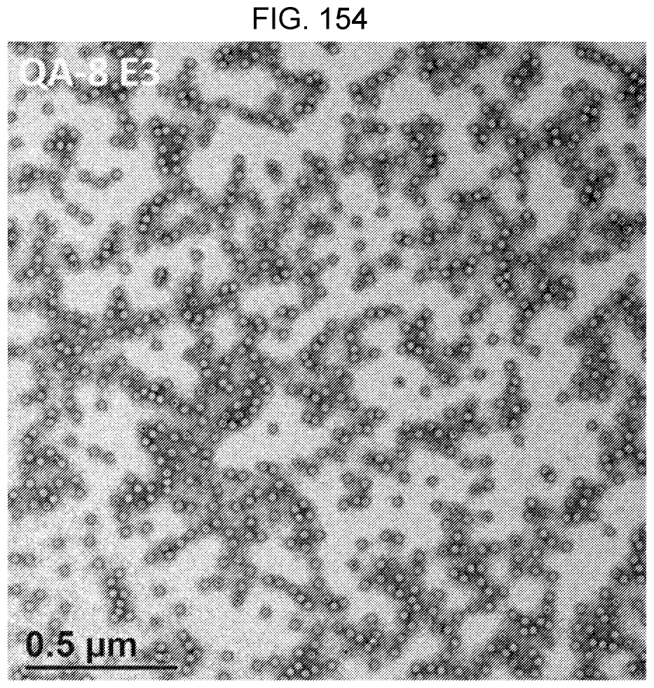

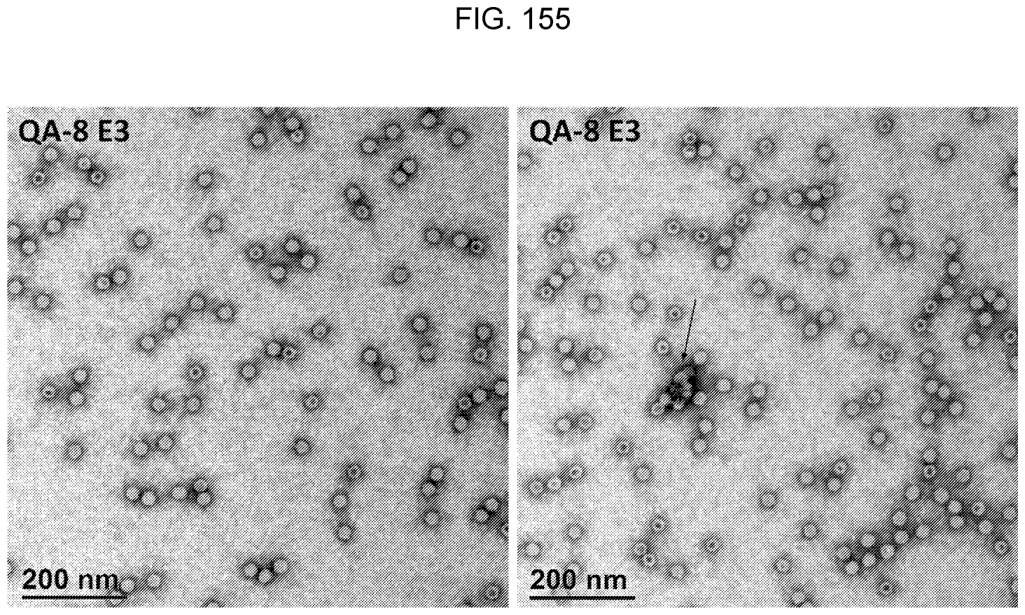

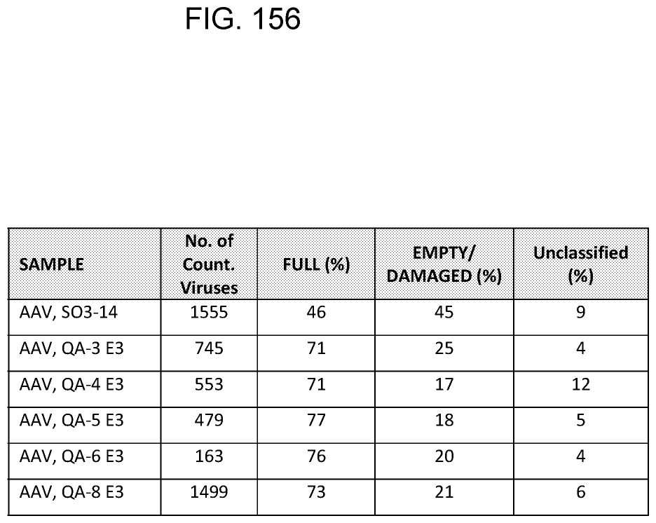

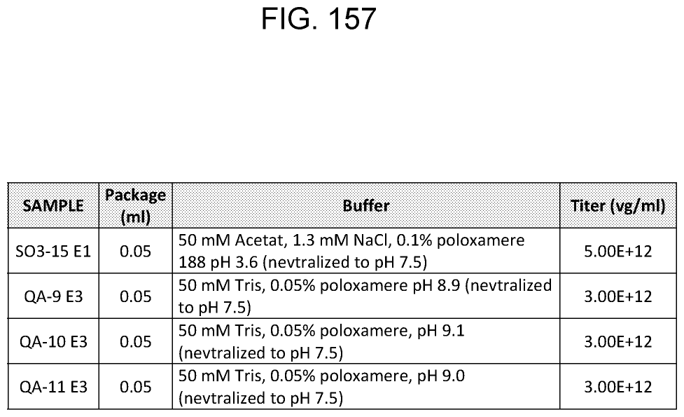

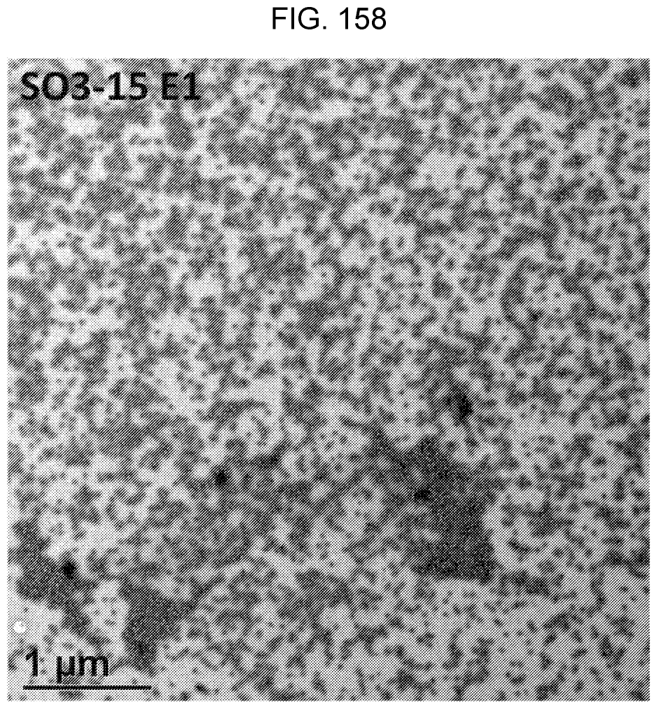

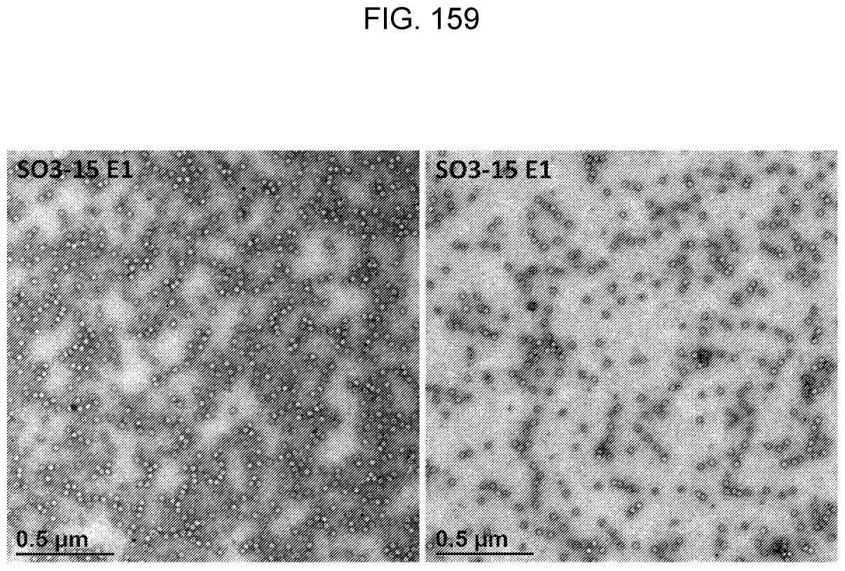

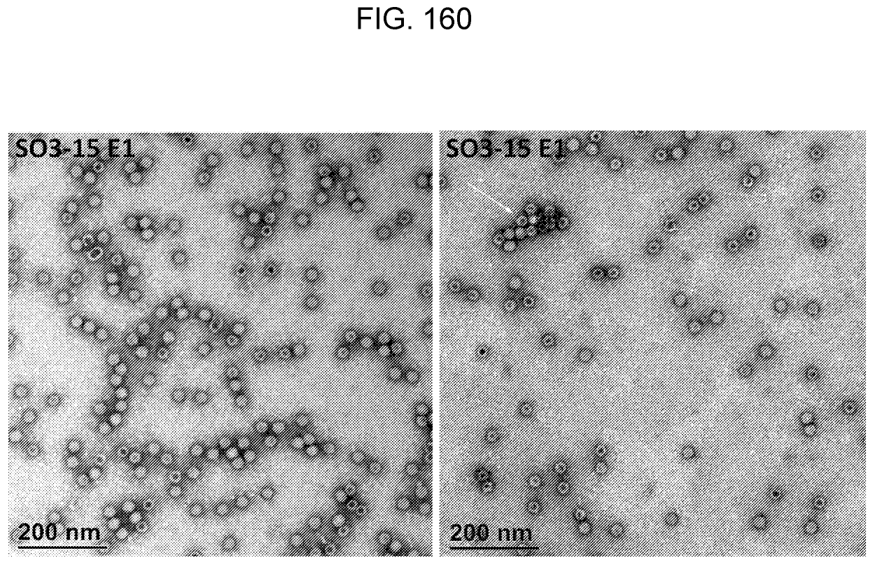

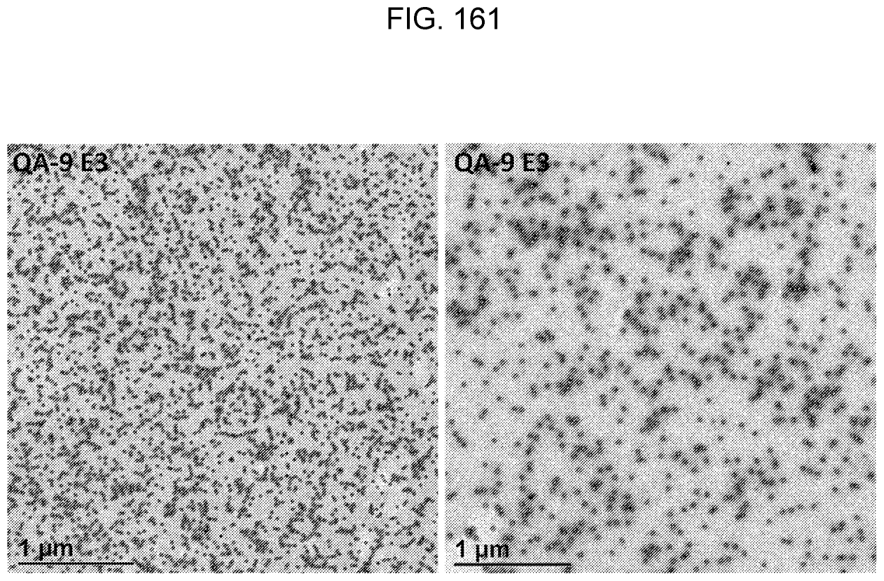

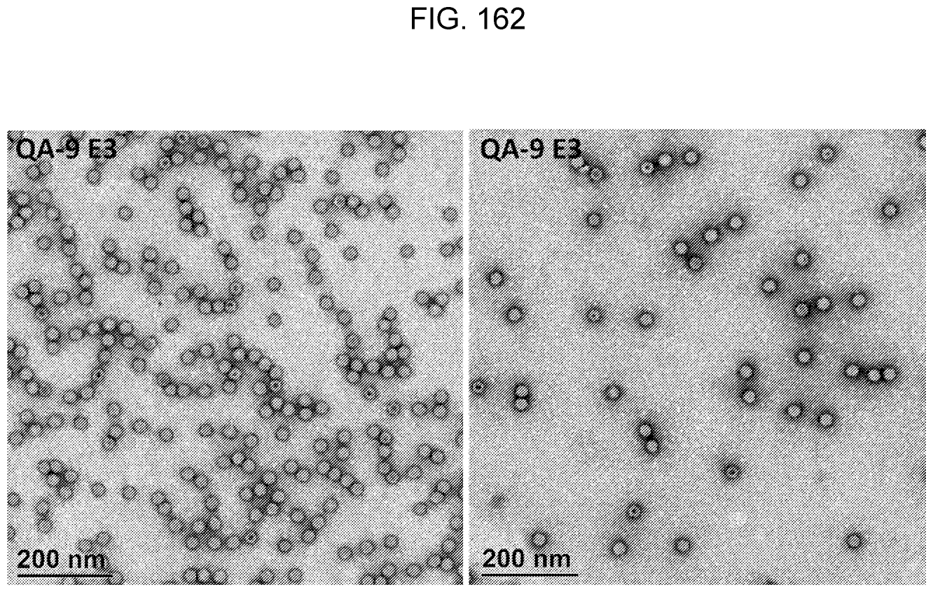

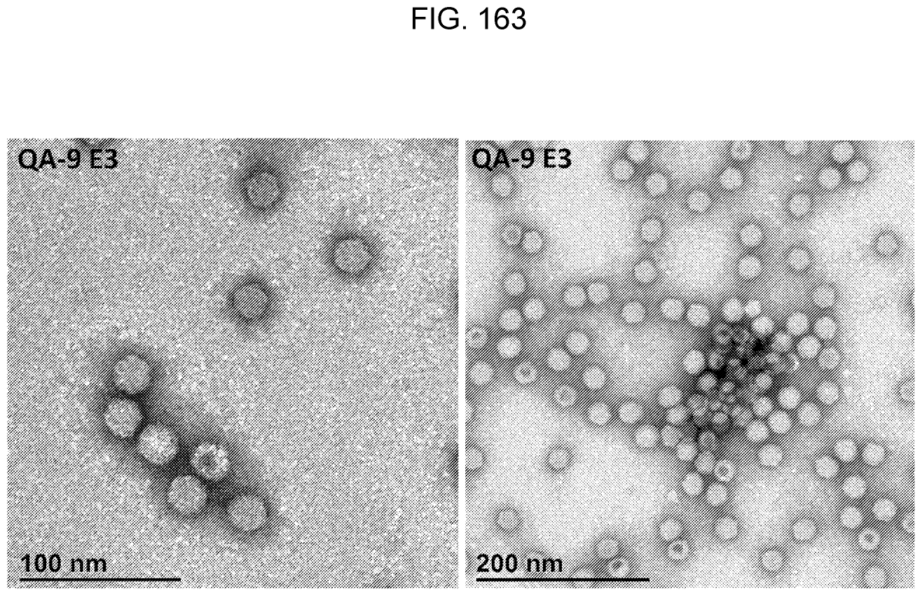

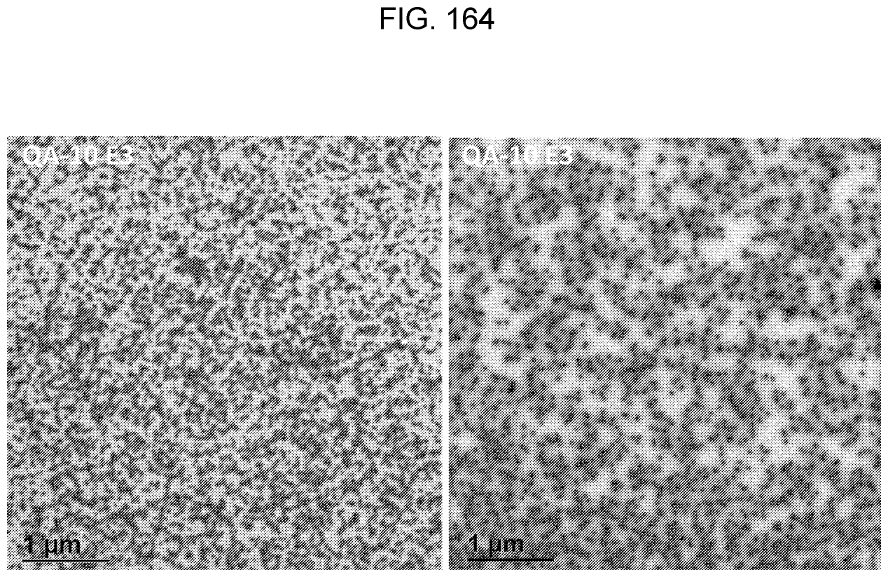

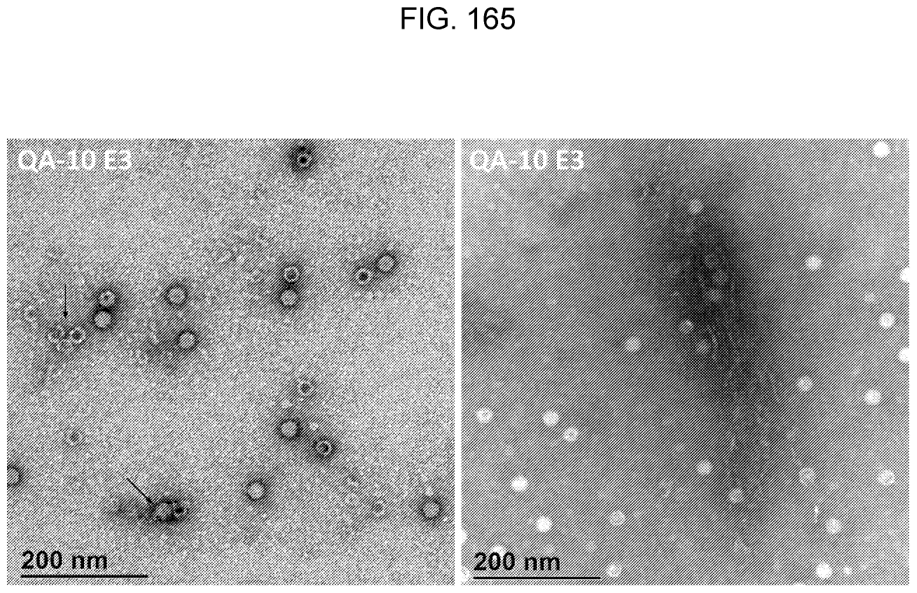

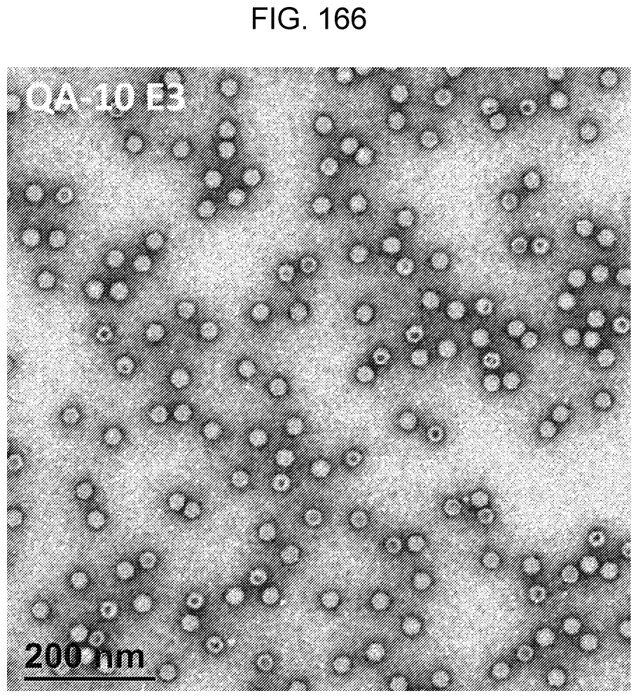

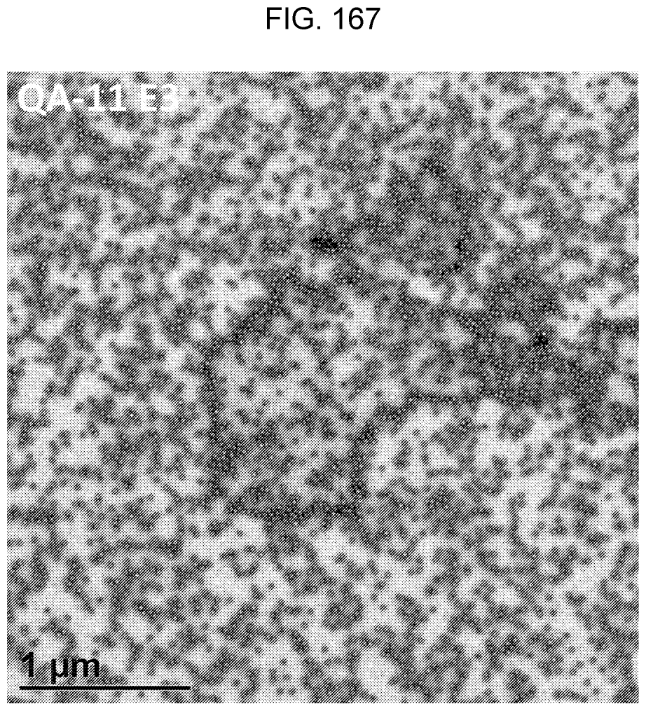

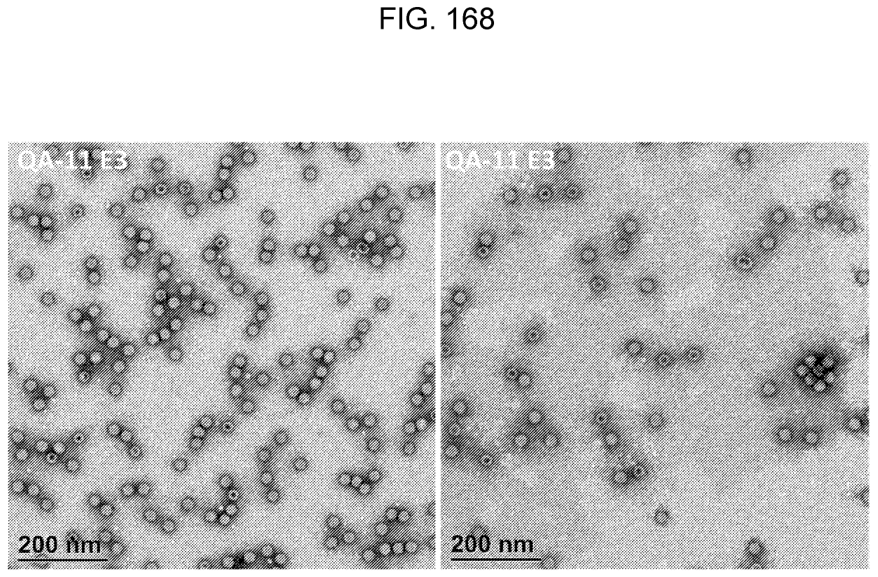

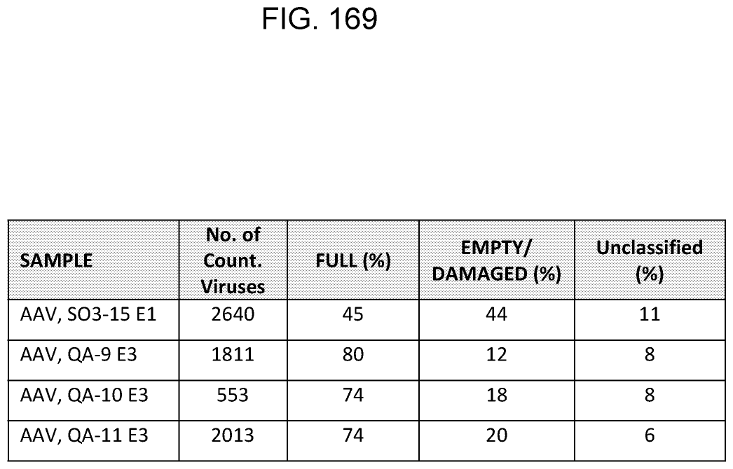

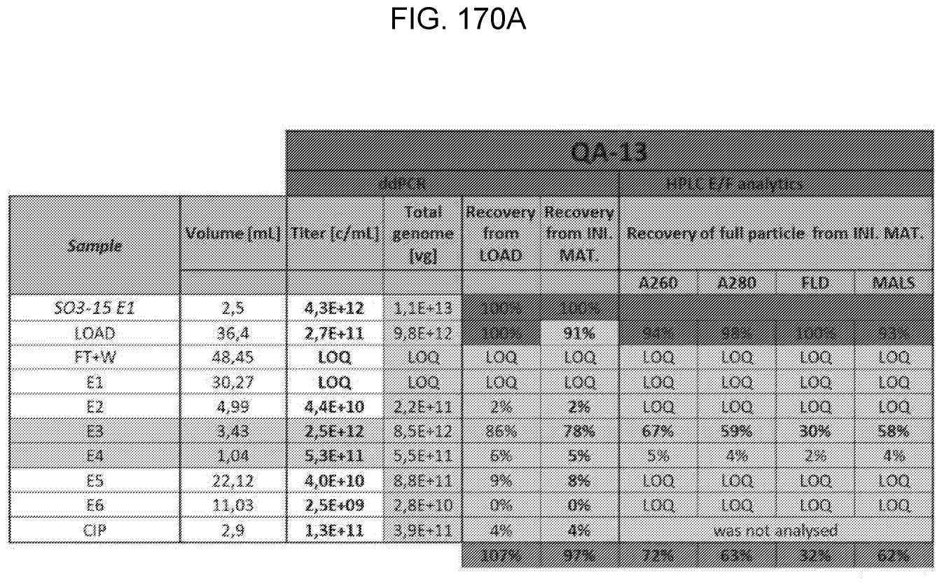

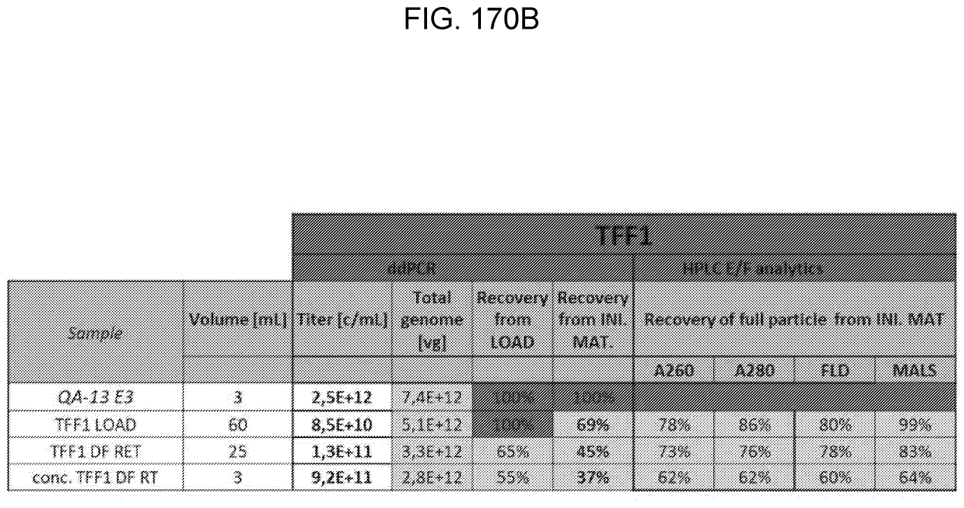

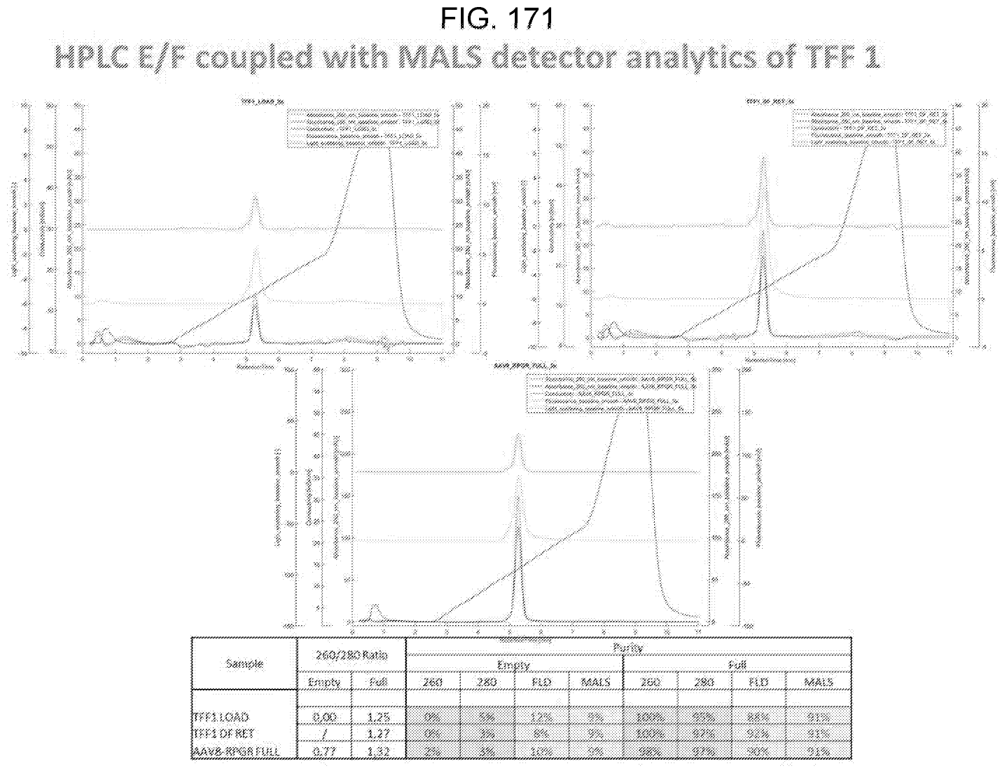

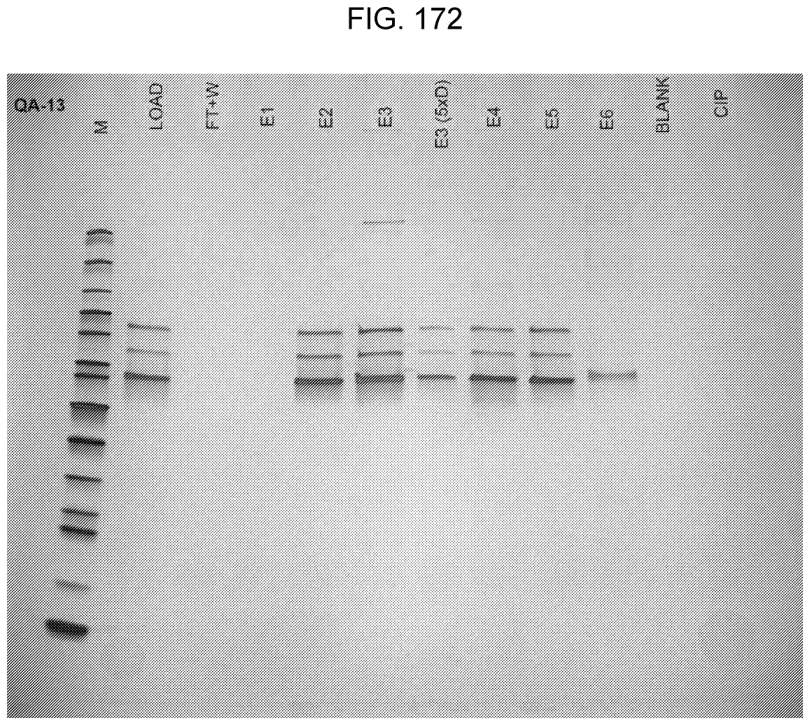

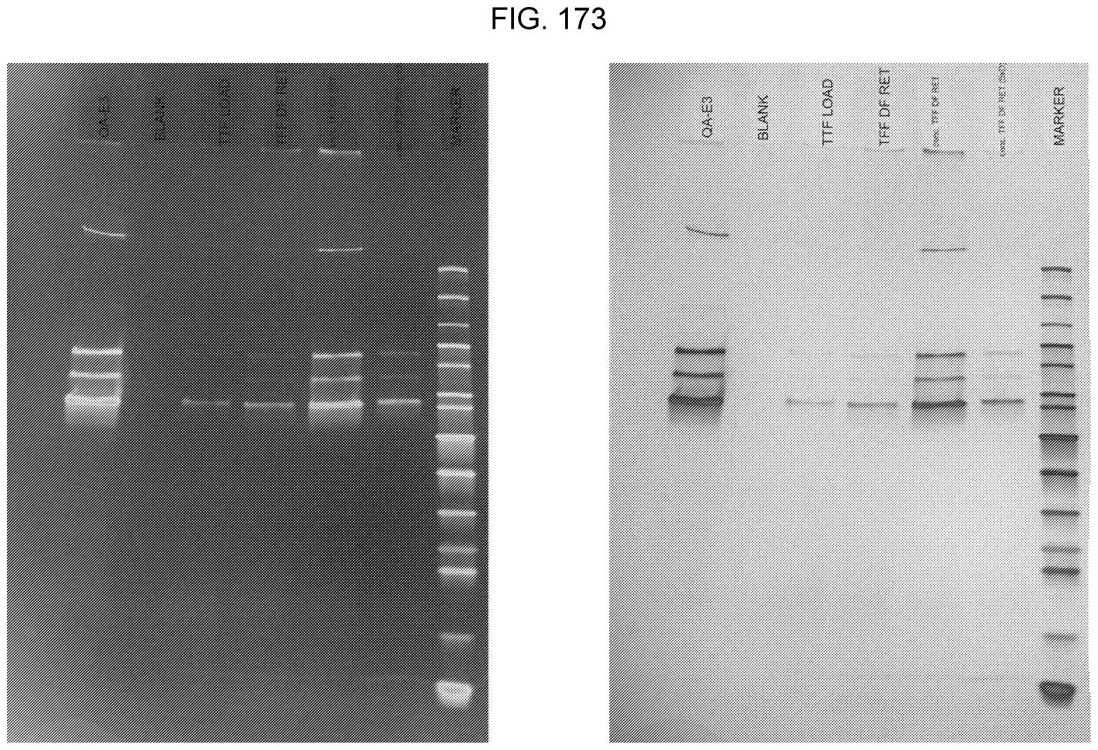

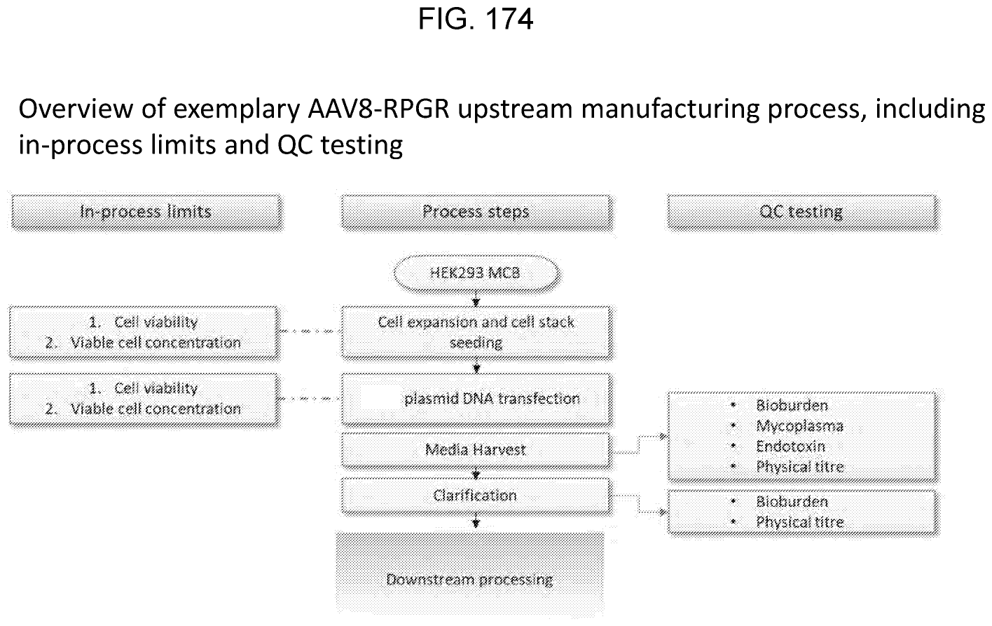

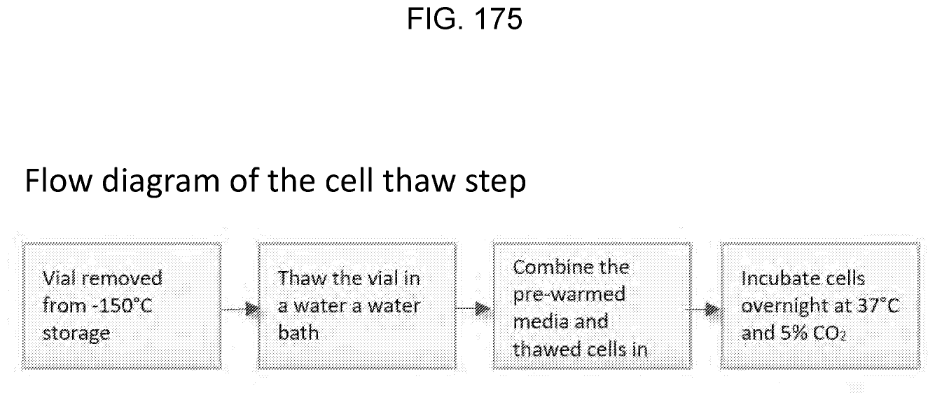

Estimated process recoveries for the process are shown in FIG. 107. The total expected yield for the three step chromatography process is between 40 and 65%. This is greater than conventional ultracentrifugation based processes.

### Example 3: AAV8-RPGR Manufacturing Process Description—Upstream and Primary Harvest Unit Operations—PD-USP-001

X-linked retinitis pigmentosa (XLRP) is a very severe form of retinitis pigmentosa (RP), resulting in rapid disease progression and severe retinal dysfunction. The worldwide prevalence of XLRP is approximately 1:30,000 to 1:40,000 (Tee et al., 2016). Patients with XLRP typically experience onset of night blindness in the first decade, followed by reduction of visual field and acuity and progressively severe visual impairment. Most patients are legally blind by the end of the fourth decade.

To date, 3 genes have been mapped to XLRP: RP2; RP3, also known as the RP GTPase regulator (RPGR) gene; and OFD1, which has been identified as a rare cause of XLRP (Webb et al., 2012). Approximately 75% of cases of XLRP are due to RPGR variants, and the worldwide prevalence of XLRP due to RPGR variants is approximately 1:40,000 to 1:53,000 (Pelletier et al., 2007; Shu et al., 2008). RPGR is involved in protein distribution in photoreceptors and plays a role in the transport of photo-transduction components and other outer segment proteins across the connecting cilium (Tee et al., 2016). Essential for photoreceptor viability, the RPGR gene product is localised in the outer segment of rod photoreceptors (Ferrari et al., 2011). Loss of RPGR function in the retina causes the progressive loss of rod and cone vision.

Nightstar Therapeutics is developing AAV8-RPGR as a potential gene therapy medicinal product (GTMP) for the treatment of XLRP due to mutations in RPGR. Replacing the deficient RPGR in XLRP patients with new and viable RPGR is expected to slow or stop retinal degeneration and maintain or improve visual function.

This document describes the upstream and primary harvest processes used for the manufacture of AAV8-RPGR product. This document includes all the upstream and primary harvest processing steps.

Manufacturing Process Description and Process Controls

Batch Definition

A batch of product defined as a single production campaign consisting of 20 Corning 36-layer HYPERStack® vessels containing plasmid DNA transfected HEK293 cells that produce the AAV8-RPGR biologic product. The cell culture media is harvested and pooled from the 20 Corning 36-HYPERStack® vessels and followed by a single purification process.

Summary of the Upstream Process

Cells are expanded using Corning flasks and stacks to allow sufficient cell mass to be generated for seeding twenty HYPERStack® units for vector production. Transfection of the cells takes place with a two-production plasmid system using an optimised calcium phosphate co-precipitation method.

After transfection, the medium is changed and Benzonase® endonuclease is added to the media to digest free genomic and plasmid DNA present in the media. To promote vector release into the media, the 36 layer HYPERStack® units are spiked with a HEPES buffered Na2HPO4 solution and incubated prior to harvest. The media from each 36-layer HYPERStack® is harvested aseptically using disposable bioprocess bags and pooled into a single volume (˜82 L).

The pooled media containing the recombinant AAV (rAAV) is then clarified using a capsule 0.65 μm pore pre-filter, followed by a 0.2 μm sterilising grade capsule filter.

Manufacturing Flow Diagram

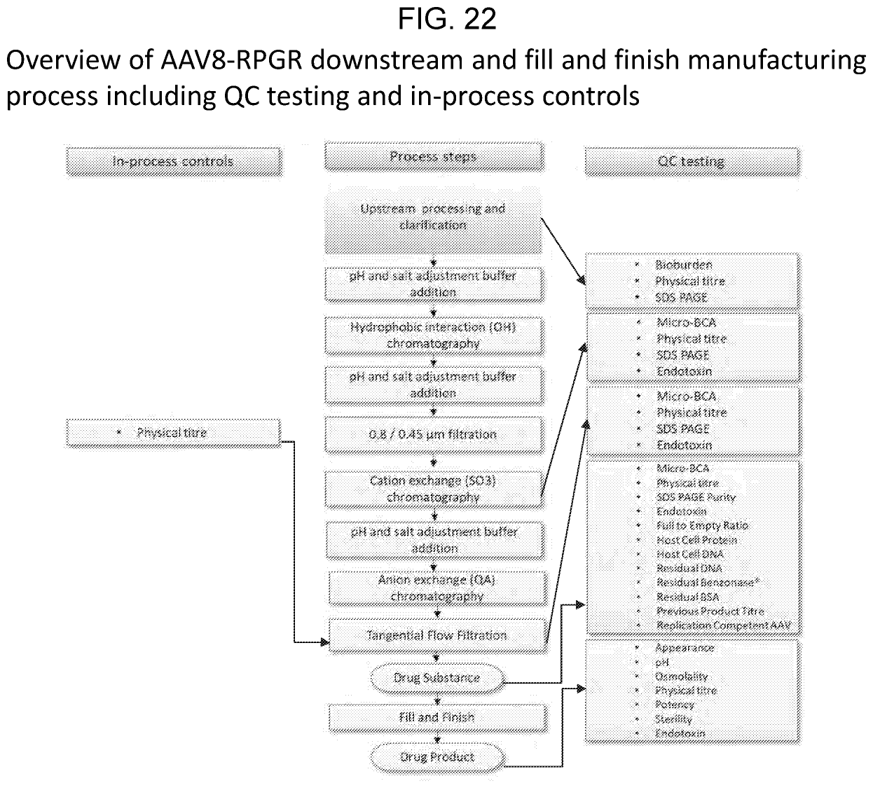

An overview of the upstream and primary recovery steps of the manufacturing process for AAV8-RPGR is illustrated in FIG. 22 along with an overview of the current process controls and QC/analytical tests performed.

Upstream Manufacturing Process Description

Vial Thaw

A vial from the HEK293 MCB is removed from −150° C. storage and subsequently thawed in a water bath that is set to a temperature of 37±1° C. A visual check is performed to ensure that the cells are thawed. It is anticipated that the WCB will be used for any further production runs.

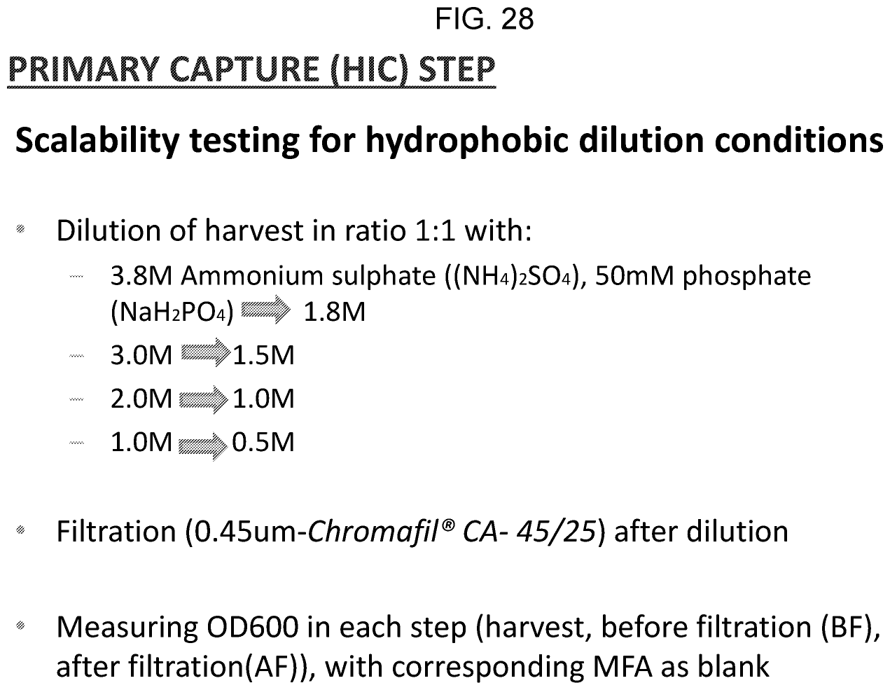

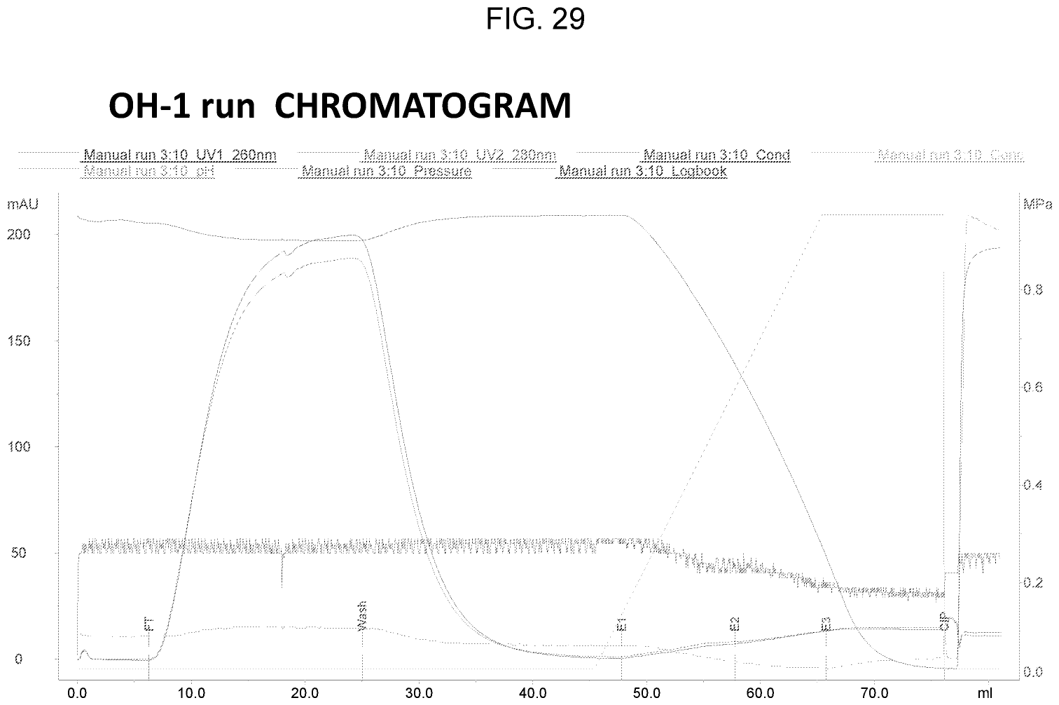

The thawed cells are added to a T-25 flask that contains 4 mL of growth media (DMEM+10% serum) that has been pre-warmed to 37±1° C. The cells are placed in a humidified CO2 incubator that is set to 37° C. and 5% CO2 and left overnight. FIG. 27 shows a flow diagram of the cell thaw step. The parameters and operating conditions to be adhered to during the cell thaw procedure are contained in FIG. 28. FIG. 29 contains details of the key materials and consumables that are required for the cell thaw process.

Cell Expansion

Cells are expanded from the initial T-25 flask through to the 36-layer HYPERStack® units through a series of passages. Cells are grown in humidified incubators set at 37° C. and 5% CO2. Cells are passaged when cell confluency reaches 80%, which is typically every three to five days.

The generic passaging protocol consists of the following:

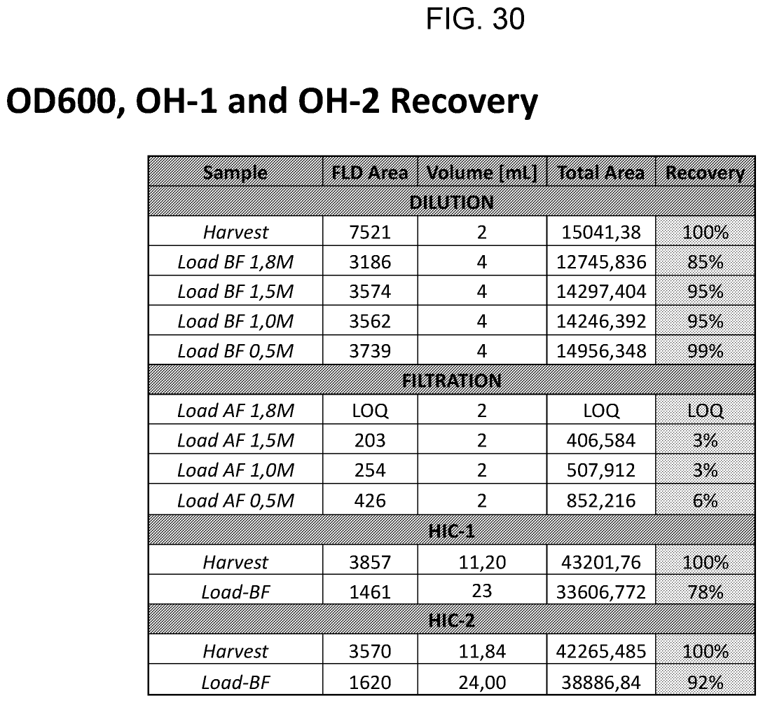

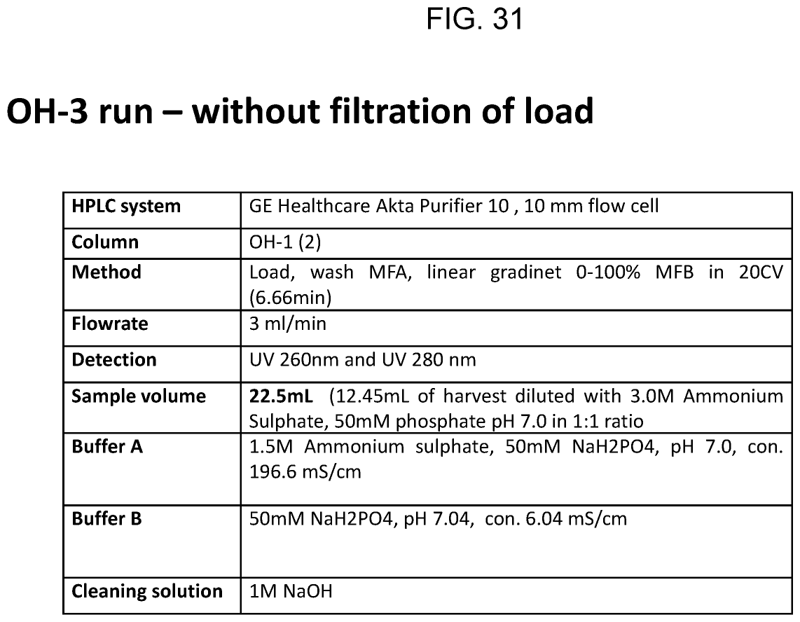

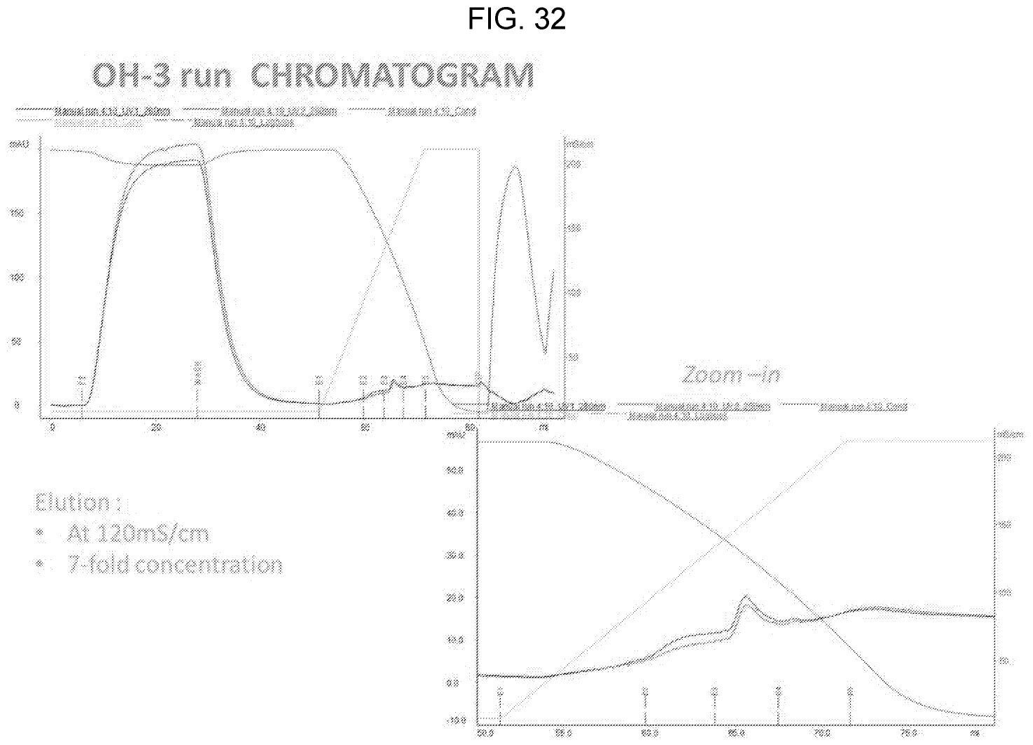

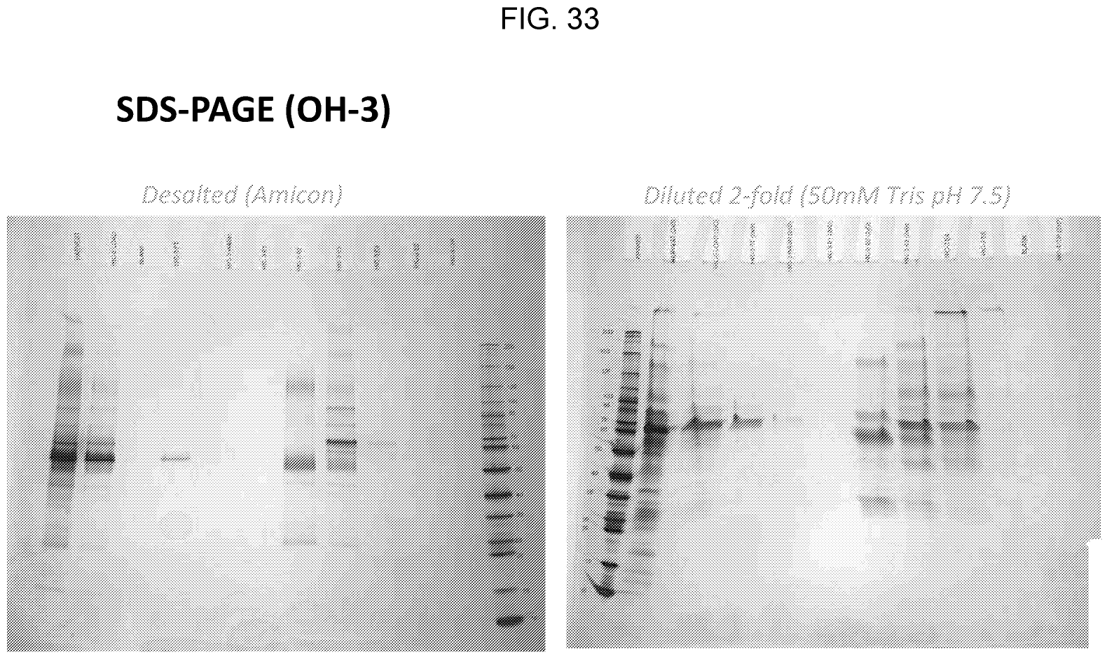

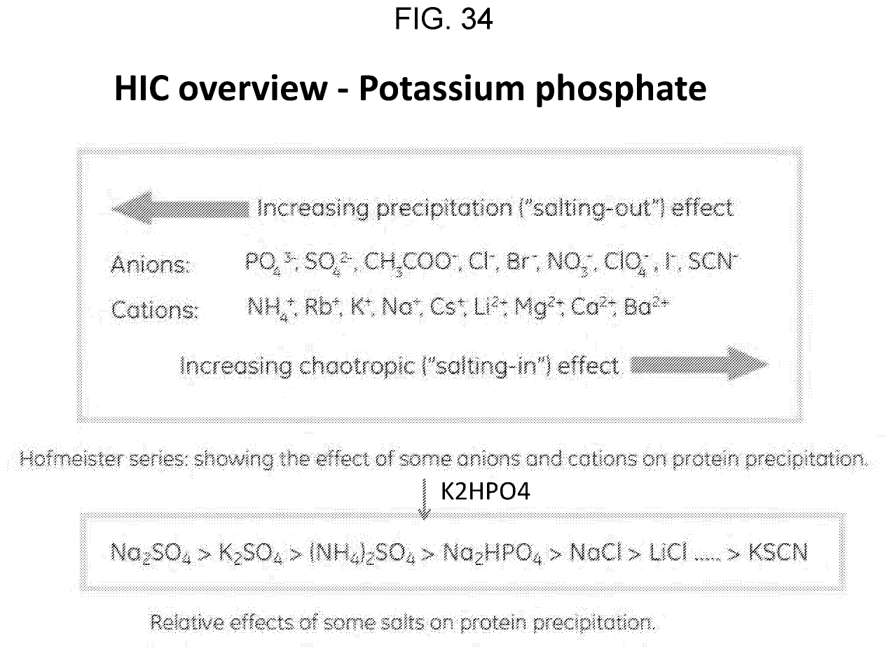

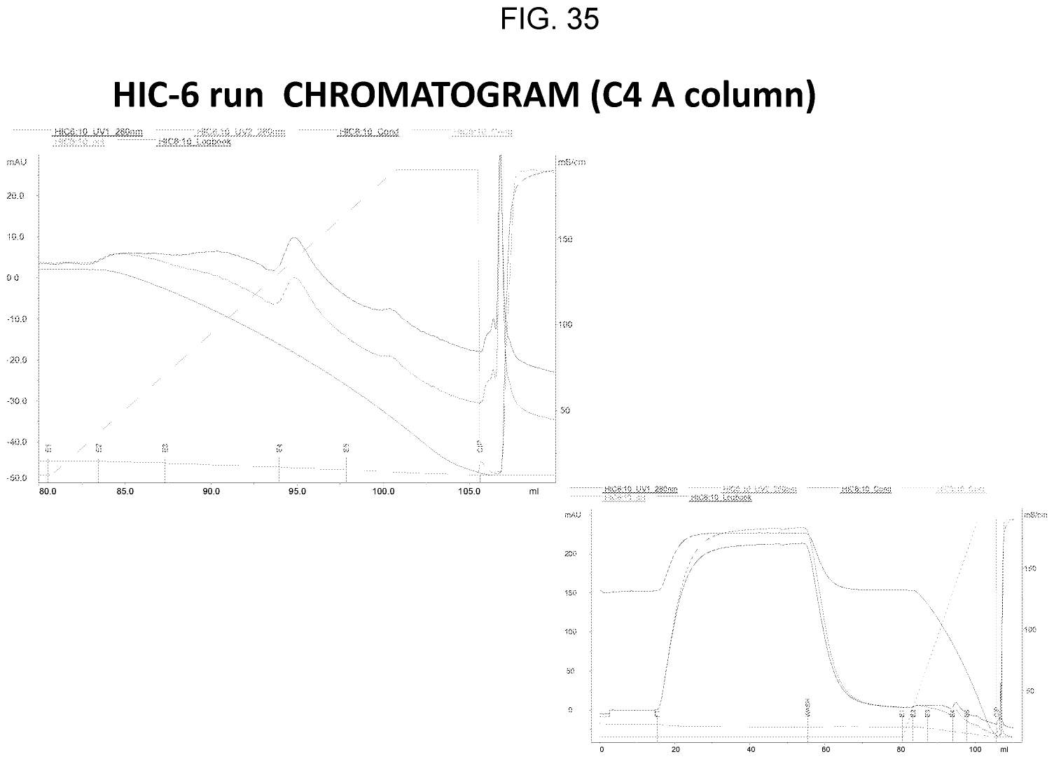

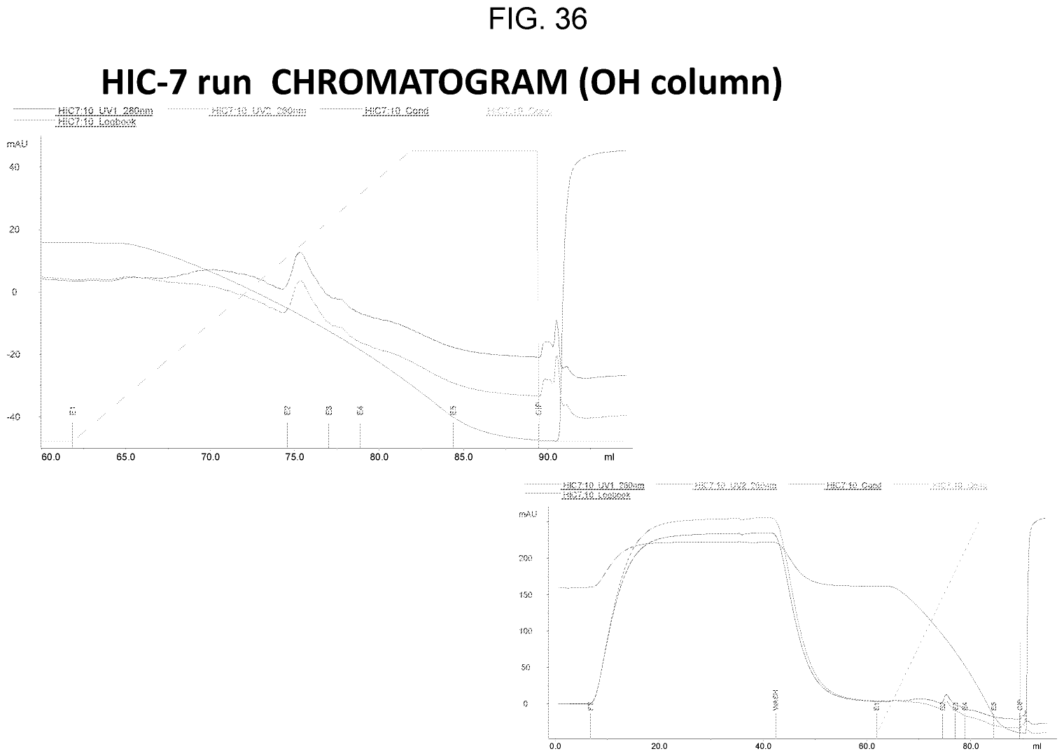

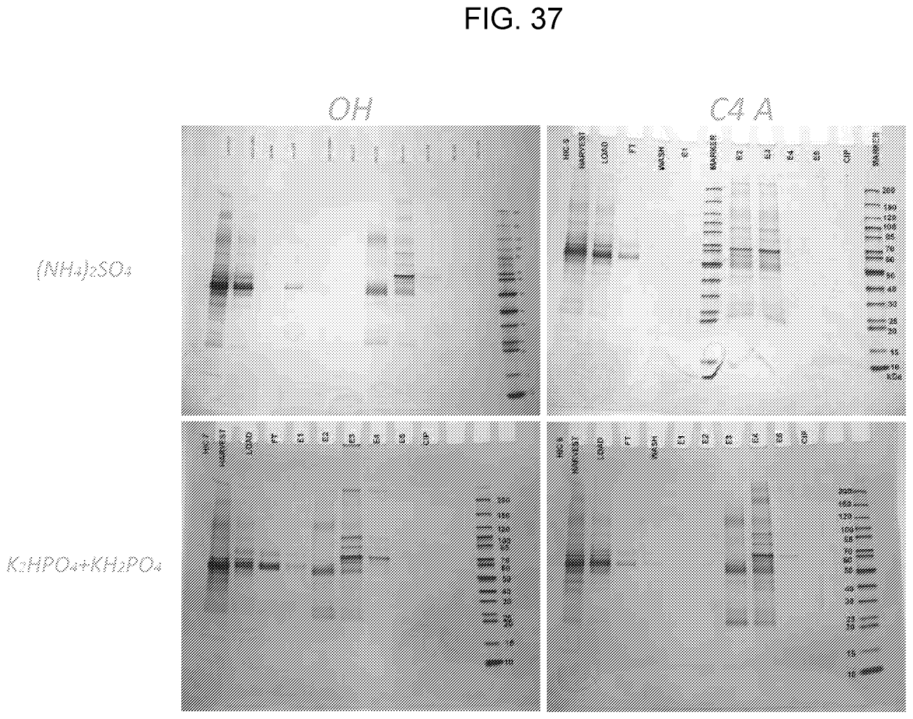

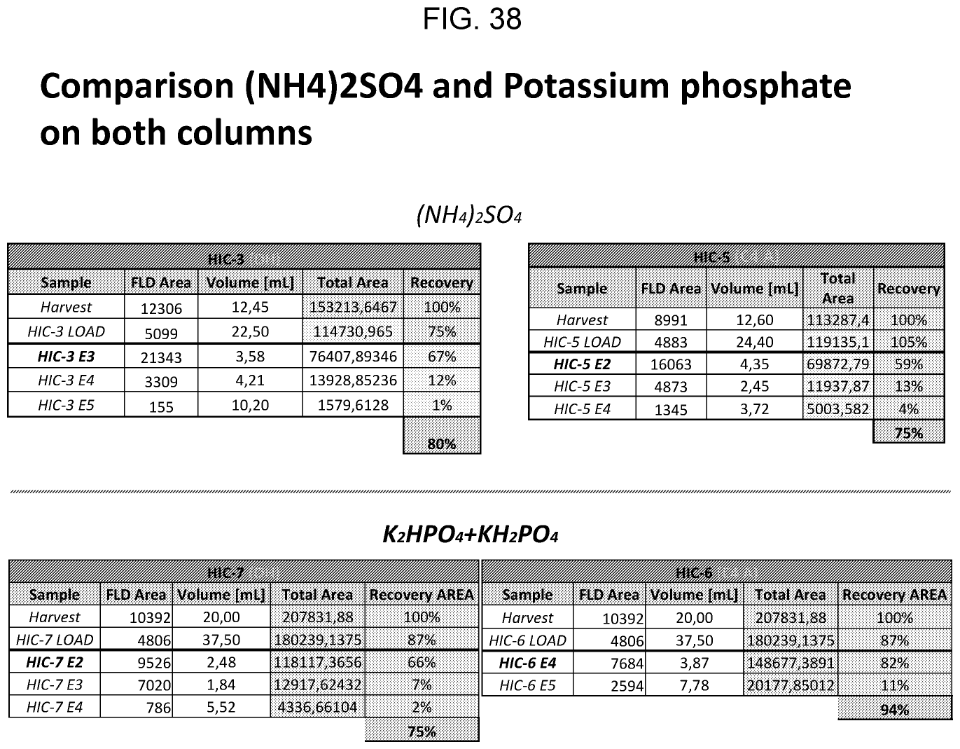

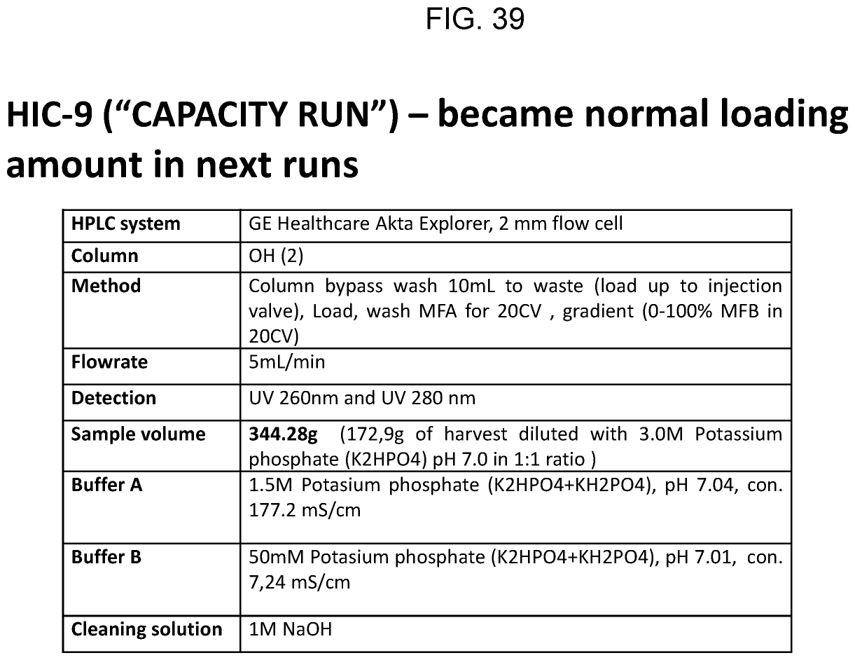

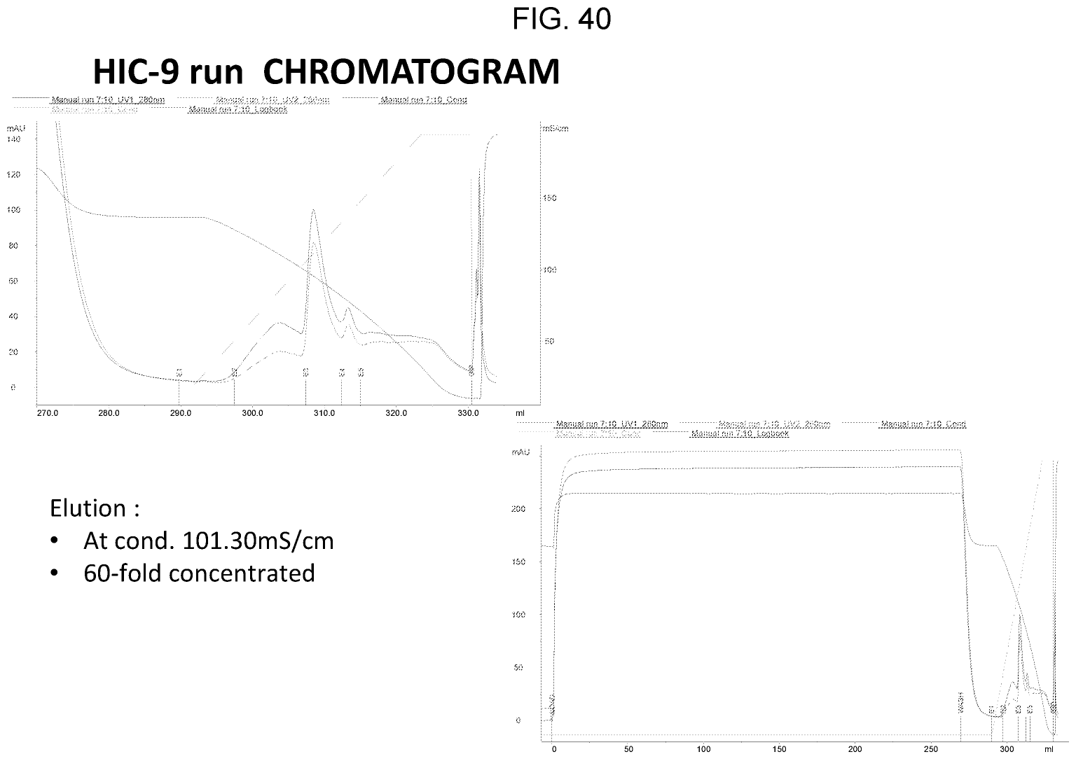

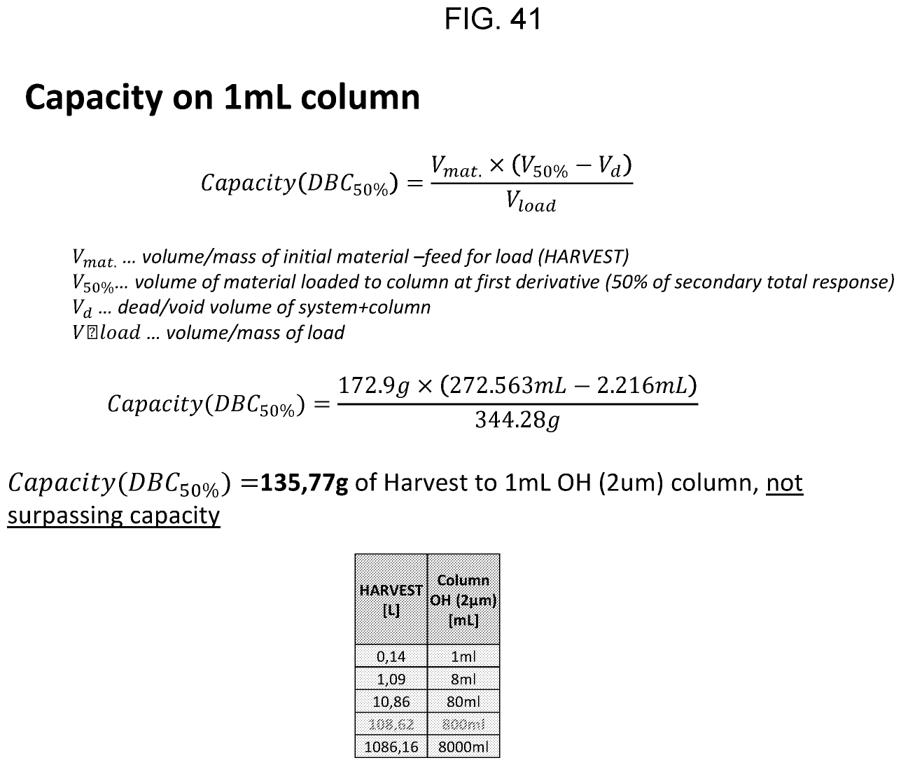

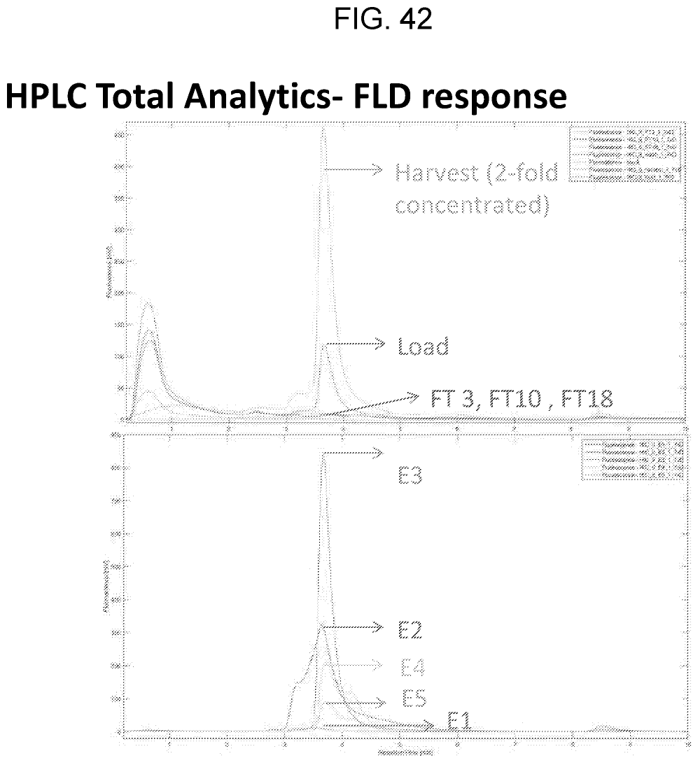

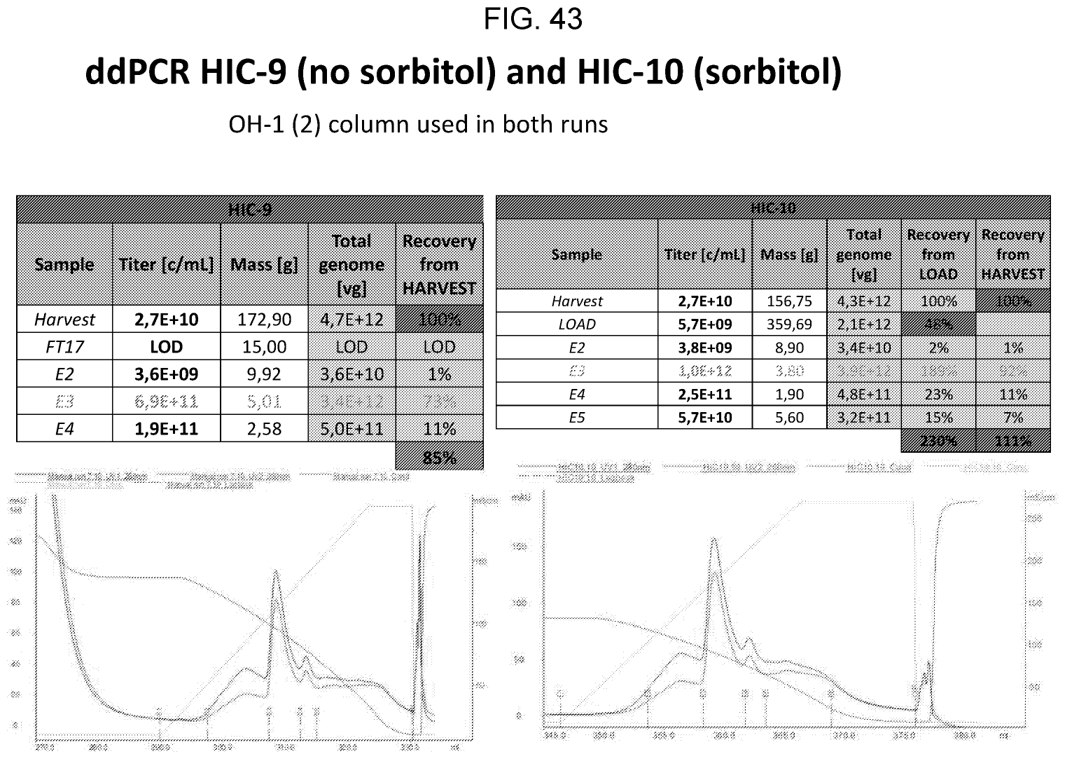

Remove media from the current cell flask/stack.
Wash the cells using pre-warmed Hanks Balanced Salt Solution (HBSS).
Add pre-warmed cell 1× dissociation solution and swirl the solution to ensure that the cell surface is covered. Remove the excess cell dissociation solution.
Incubate the cell flask/stack for a further 3-5 minutes. Dislodge the cells using careful manual tapping.
Add further growth media to help remove the cells.
Remove the cells into an intermediate storage container.
Combine the cell suspension with new pre-warmed growth media
Seed the new cell flask/stack.
Incubate the cells at 37° C. and 5% CO2.
   FIG. 30 provides a high-level summary of the generic passage procedure whilst FIG. 31 details the generic criteria for cell passages. FIG. 32 contains the recommended volumes and seeding densities, related to passages, for each possible cell culture vessel, up to the 36-layer HYPERStack® unit. The recommended minimum warming times are contained in FIG. 33. FIG. 34 contains details of the key materials and consumables that are required for the cell thaw and routine passage steps. Cells are recommended to be sub-cultured for approximately 30 passages. When approaching their useful passage limit, a new vial should be thawed before the old cells are discarded.

Transient Transfection, Benzonase® Addition, Media Release and Harvesting

Following 3 days of growth post seeding, the HYPERStack® media is replaced with fresh DMEM media containing serum and chloroquine; this is performed 2-8 hours before transfection. The cells are then transfected with the two production plasmids using an optimized calcium phosphate co-precipitation method.

Sufficient DNA plasmid transfection precipitate is prepared in a biological safety cabinet to transfect 5×36-layer HYPERStacks®. Initially a DNA/calcium mix is prepared containing the vector plasmid, the pDP8.ape helper plasmid and CaCl₂). After mixing well, the plasmid/CaCl₂) solution is added to an equal volume of 2×HEPES buffered NaHPO₄ with concurrent gentle agitation in a disposable process bag to obtain an optimal precipitate. The solution is sat at room temperature for at least 5 minutes and then added to the five 5 HYPERStacks® linked with a manifold. This procedure is repeated four times to complete the transfection of the required 20 HYPERStack® units.

Post transfection, the cells are incubated in an incubator set at 37° C. and 5% CO2. Approximately 22 hours after transfection, the medium is changed using serum-free DMEM. At this time the Benzonase® endonuclease is added to the media, at a concentration of 90 U/mL, to digest free genomic DNA and plasmid DNA present in the media. This step is performed to minimize the amount of residual host cell DNA in the final vector product. The cells are then incubated in an incubator set at 37° C. and 5% CO2 for an additional 69-75 hours. To promote vector release, the 36-layer HYPERStacks® are spiked with a HEPES buffered NaHPO4 solution and incubated for approximately 18 hours in an incubator set at 39° C. and 5% CO2 prior to harvest. The media from each 36-layer HYPERStack® is harvested aseptically using disposable bioprocess bags and pooled into a single volume (˜82 L). FIG. 182 shows a flow diagram of the transient transfection and media harvest steps. FIG. 183 contains the volumes of chloroquine per cell culture unit and FIG. 184 details the operating ranges for the transfection and harvest steps. FIG. 185 contains the details of the key consumables and materials used in the transfection process.

Clarification by Filtration

The pooled media containing the recombinant AAV (rAAV) is then clarified through a capsule pre-filter, followed by a sterilising grade capsule filter. A second back-up pre-filter is installed as part of the filtration set up and is to be used if the inlet pressure reaches 10 psi when the first pre-filter is in use. The pre-filter has a pore size of 0.65 μm and is constructed of, glass fibre. The bioburden reduction filter is a 0.2 μm sterilizing grade filter constructed of polyethersulfone (PES). To achieve maximal recovery, after product filtration, filters are blown down aseptically and chased with buffer. FIG. 186 shows a flow diagram of the filtration clarification step. FIG. 187 contains the operating parameters for the filtration clarification unit operation. FIG. 188 shows the key materials/consumables used in the clarification filtration step.

Preferred Chemicals for Solution Preparation

Compendial or multi-compendial chemicals are to be used wherever possible. FIG. 208 provides a list of the preferred chemicals and associated grades that have been used in the process.

### Example 4: AAV8-RPGR Manufacturing Process Description—Upstream and Primary Harvest Unit Operations—PD-USP-002

X-linked retinitis pigmentosa (XLRP) is a very severe form of retinitis pigmentosa (RP), resulting in rapid disease progression and severe retinal dysfunction. The worldwide prevalence of XLRP is approximately 1:30,000 to 1:40,000 (Tee et al., 2016). Patients with XLRP typically experience onset of night blindness in the first decade, followed by reduction of visual field and acuity and progressively severe visual impairment. Most patients are legally blind by the end of the fourth decade.

To date, 3 genes have been mapped to XLRP: RP2; RP3, also known as the RP GTPase regulator (RPGR) gene; and OFD1, which has been identified as a rare cause of XLRP (Webb et al., 2012). Approximately 75% of cases of XLRP are due to RPGR variants, and the worldwide prevalence of XLRP due to RPGR variants is approximately 1:40,000 to 1:53,000 (Pelletier et al., 2007; Shu et al., 2008). RPGR is involved in protein distribution in photoreceptors and plays a role in the transport of photo-transduction components and other outer segment proteins across the connecting cilium (Tee et al., 2016). Essential for photoreceptor viability, the RPGR gene product is localised in the outer segment of rod photoreceptors (Ferrari et al., 2011). Loss of RPGR function in the retina causes the progressive loss of rod and cone vision.

Nightstar Therapeutics is developing AAV8-RPGR as a potential gene therapy medicinal product (GTMP) for the treatment of XLRP due to mutations in RPGR. Replacing the deficient RPGR in XLRP patients with new and viable RPGR is expected to slow or stop retinal degeneration and maintain or improve visual function.

This document describes the upstream and primary harvest processes used for the manufacture of AAV8-RPGR product. This document includes all the upstream and primary harvest processing steps.

Manufacturing Process Description and Process Controls

Batch Definition

A batch of product defined as a single production campaign consisting of 20 Corning 36-layer HYPERStack® vessels containing plasmid DNA transfected HEK293 cells that produce the AAV8-RPGR biologic product. The cell culture media is harvested and pooled from the 20 Corning 36-HYPERStack® vessels and followed by a single purification process.

Summary of the Upstream Process

Cells are expanded using Corning flasks and stacks to allow sufficient cell mass to be generated for seeding twenty HYPERStack® units for vector production. Transfection of the cells takes place with a three-production plasmid system using either an optimized calcium phosphate or PEIpro® mediated method.

After transfection, the medium is changed and Benzonase® endonuclease is added to the media to digest free genomic and plasmid DNA present in the media. To promote vector release into the media, the 36-layer HYPERStack® units are spiked with a HEPES buffered Na2HPO4 solution and incubated prior to harvest. The media from each 36-layer HYPERStack® is harvested aseptically using disposable bioprocess bags and pooled into a single volume (˜82 L).

The pooled media containing the recombinant AAV (rAAV) is then clarified using a capsule 0.65 μm pore pre-filter, followed by a 0.2 μm sterilising grade capsule filter.

Manufacturing Flow Diagram

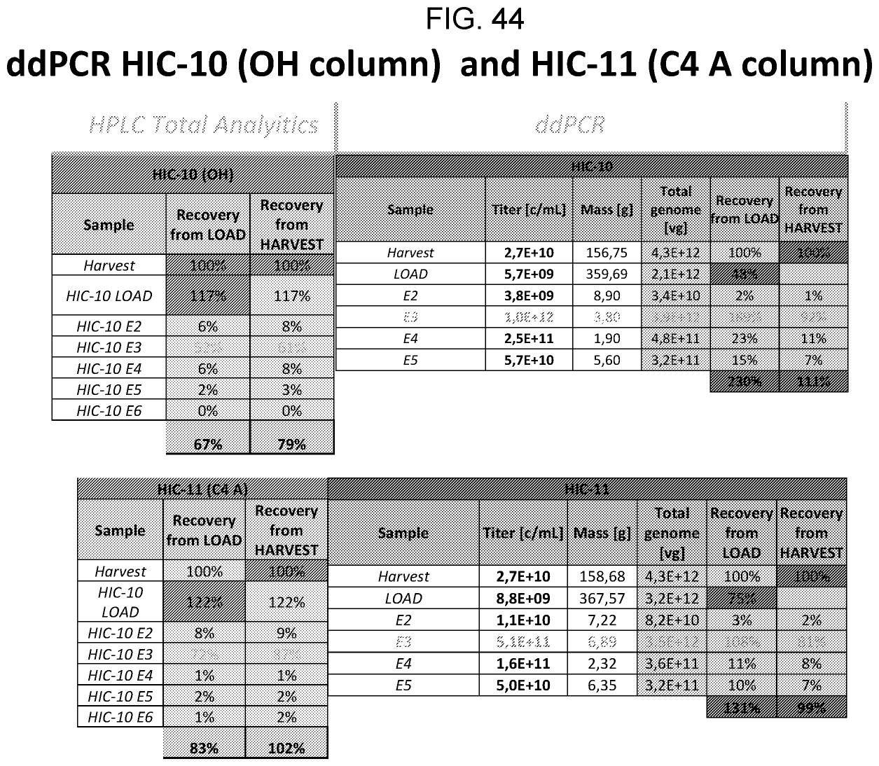

An overview of the upstream and primary recovery steps of the manufacturing process for AAV8-RPGR is illustrated in FIG. 44 along with an overview of the current process controls and QC/analytical tests performed.

Upstream Manufacturing Process Description

Vial Thaw

A vial from the HEK293 MCB is removed from −150° C. storage and subsequently thawed in a water bath that is set to a temperature of 37±1° C. A visual check is performed to ensure that the cells are thawed. It is anticipated that the WCB will be used for any further production runs.

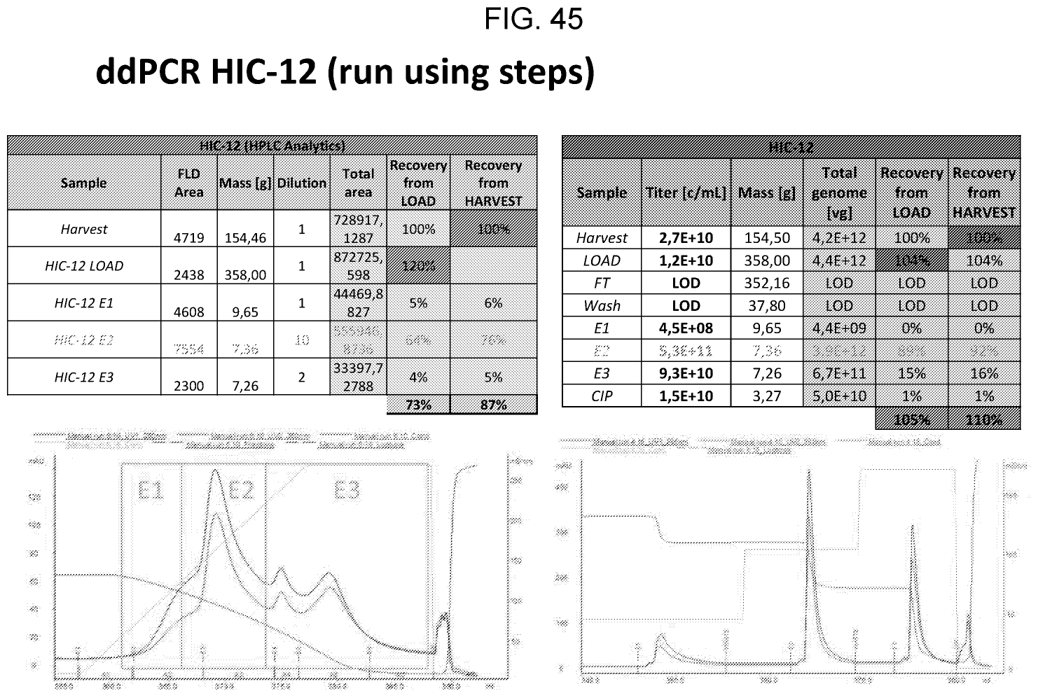

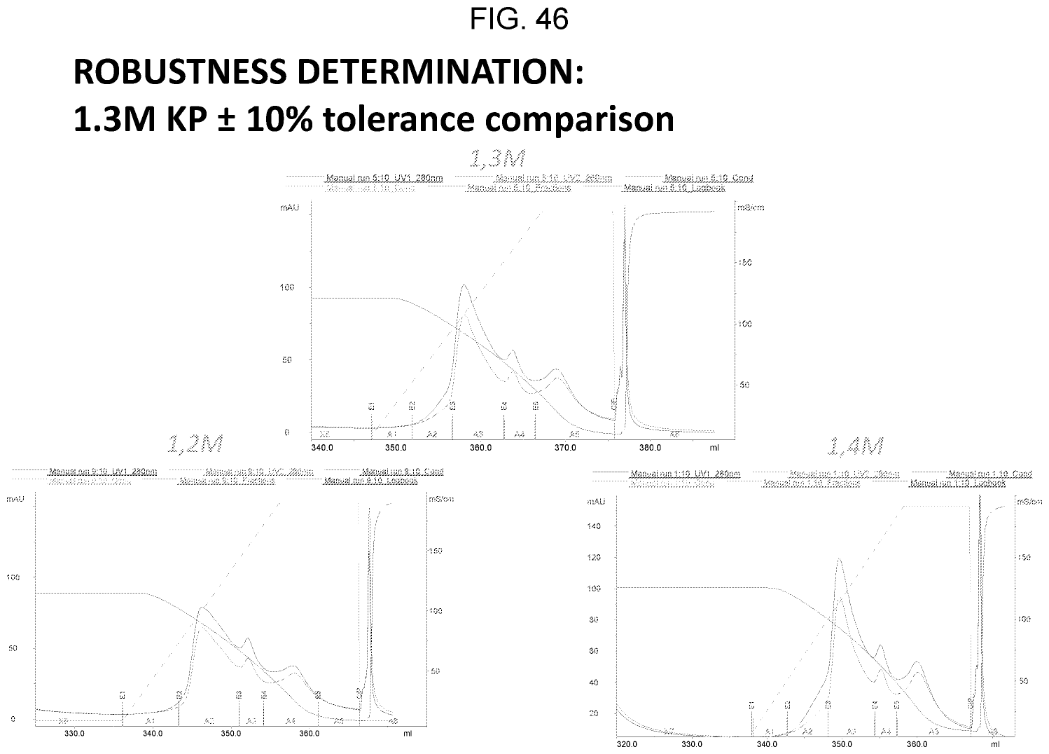

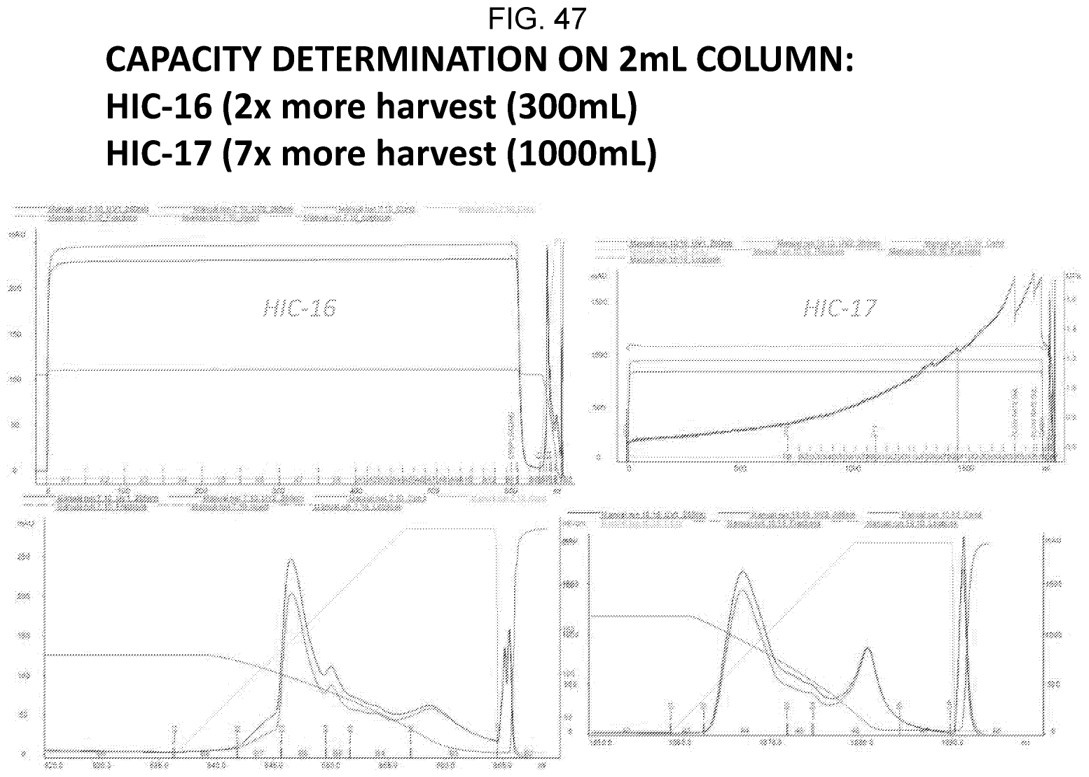

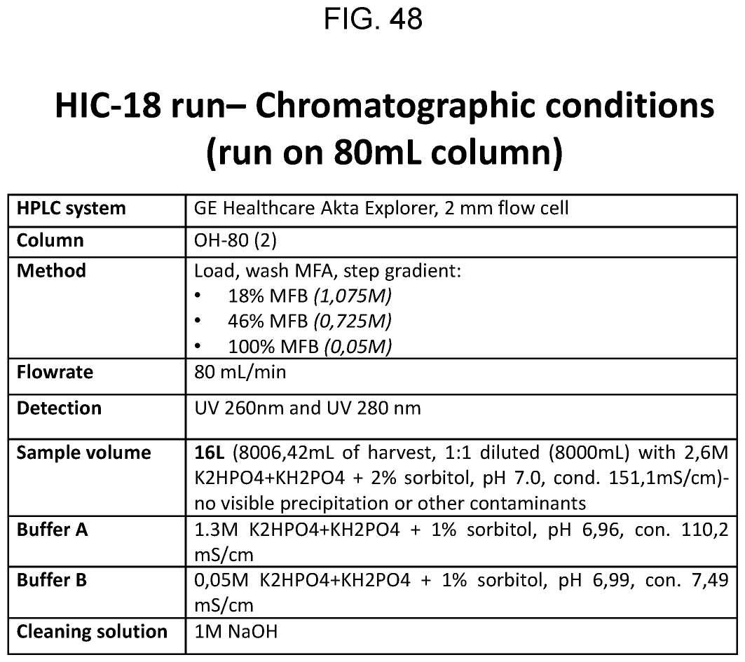

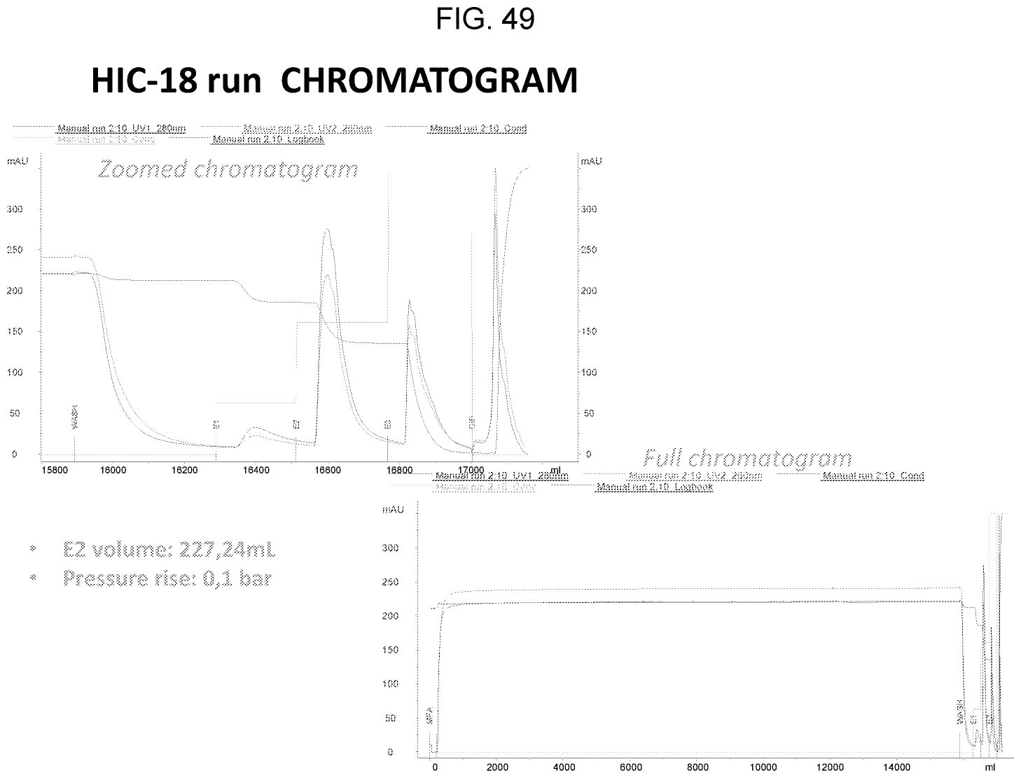

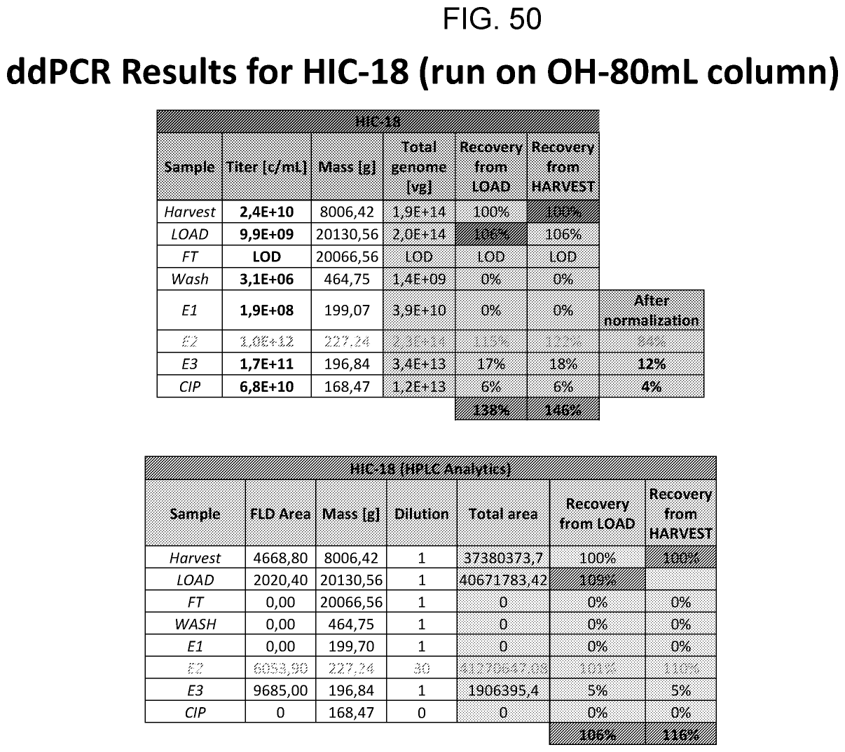

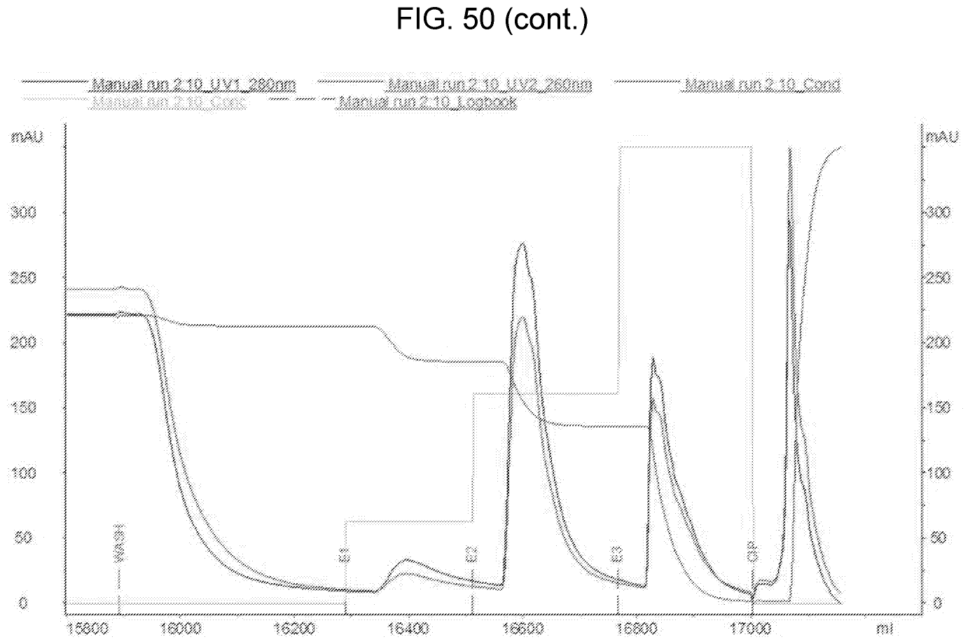

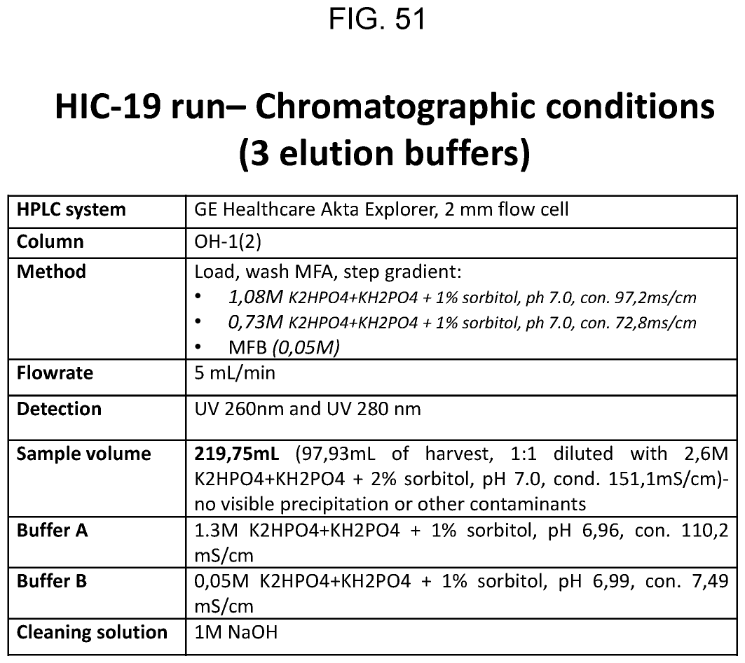

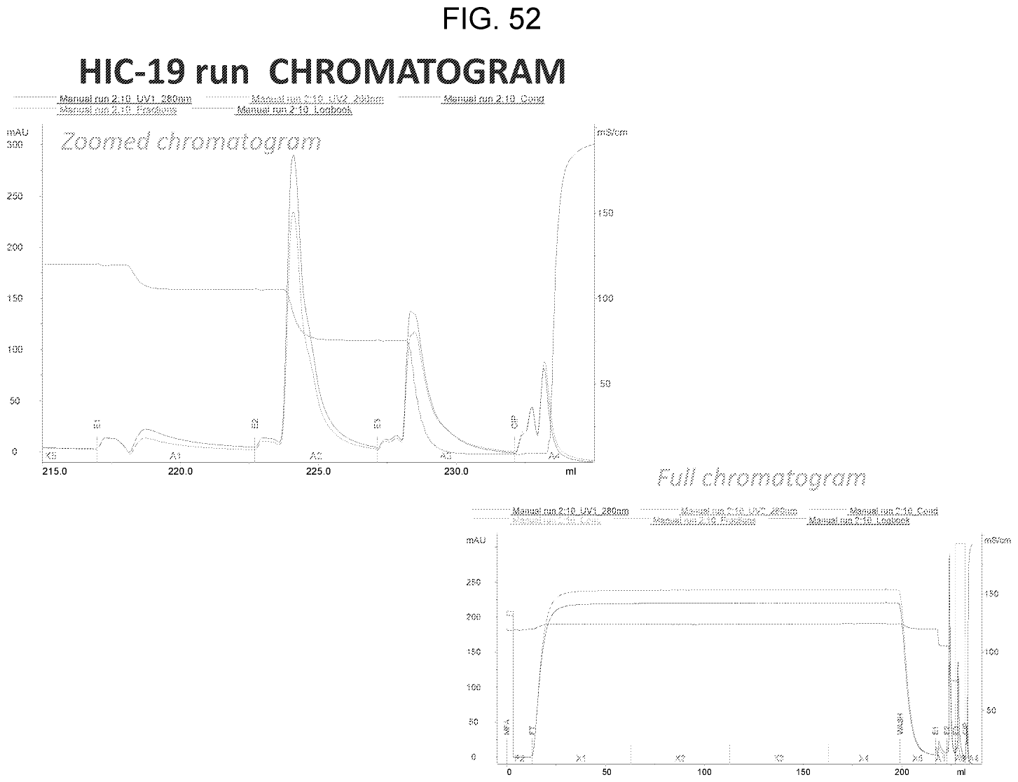

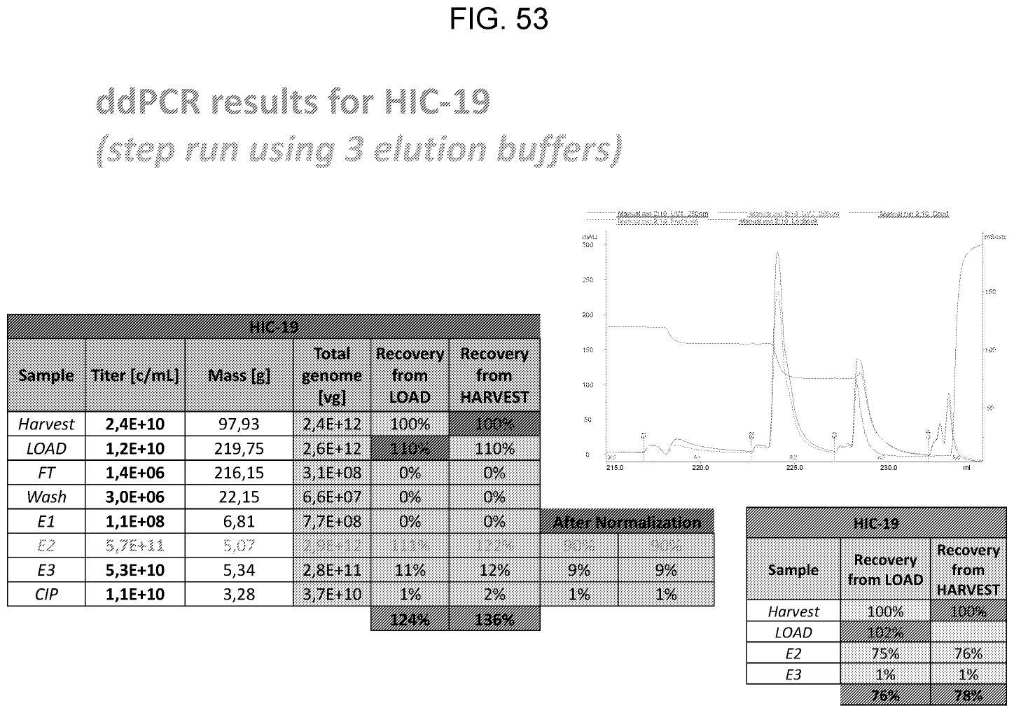

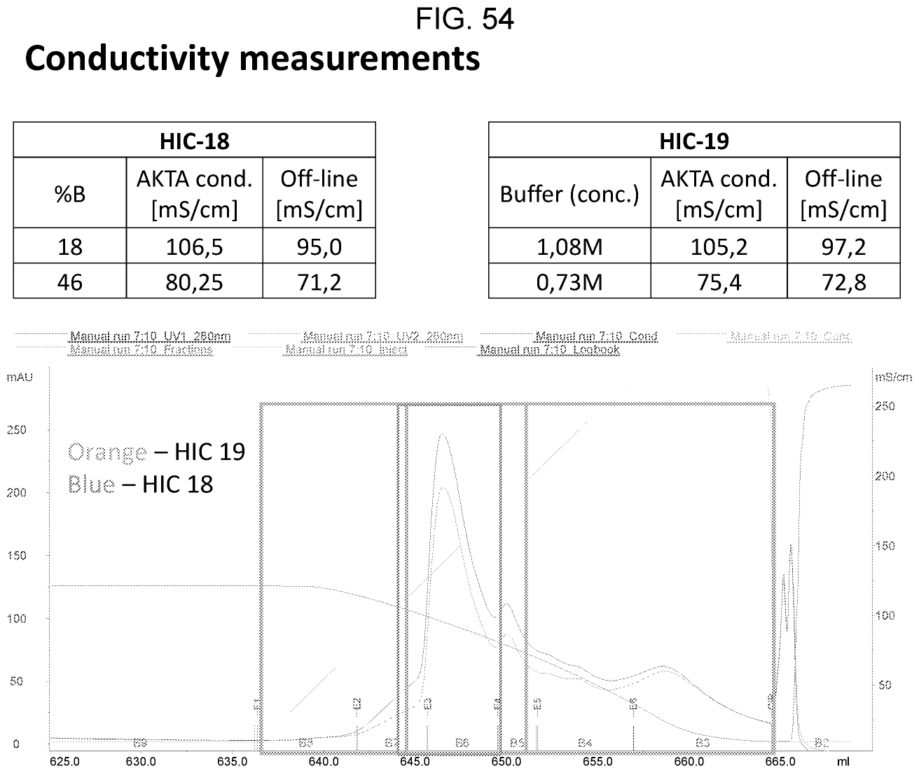

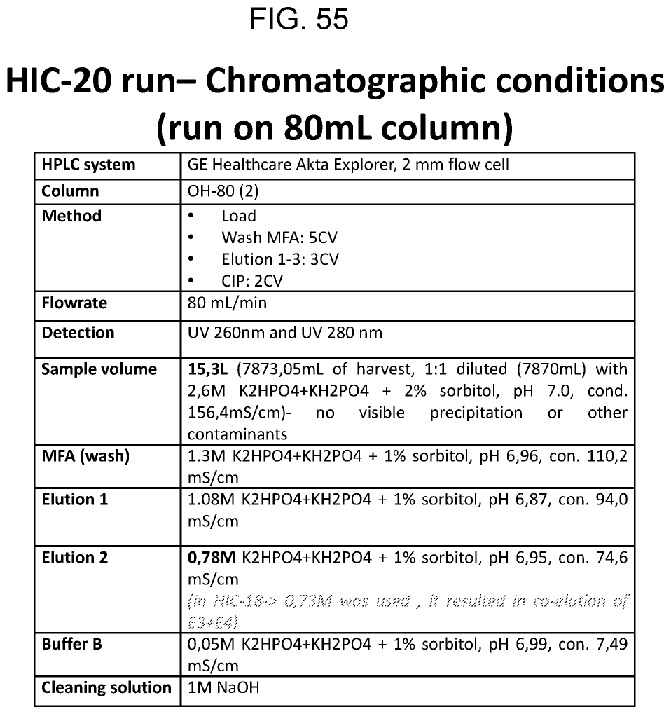

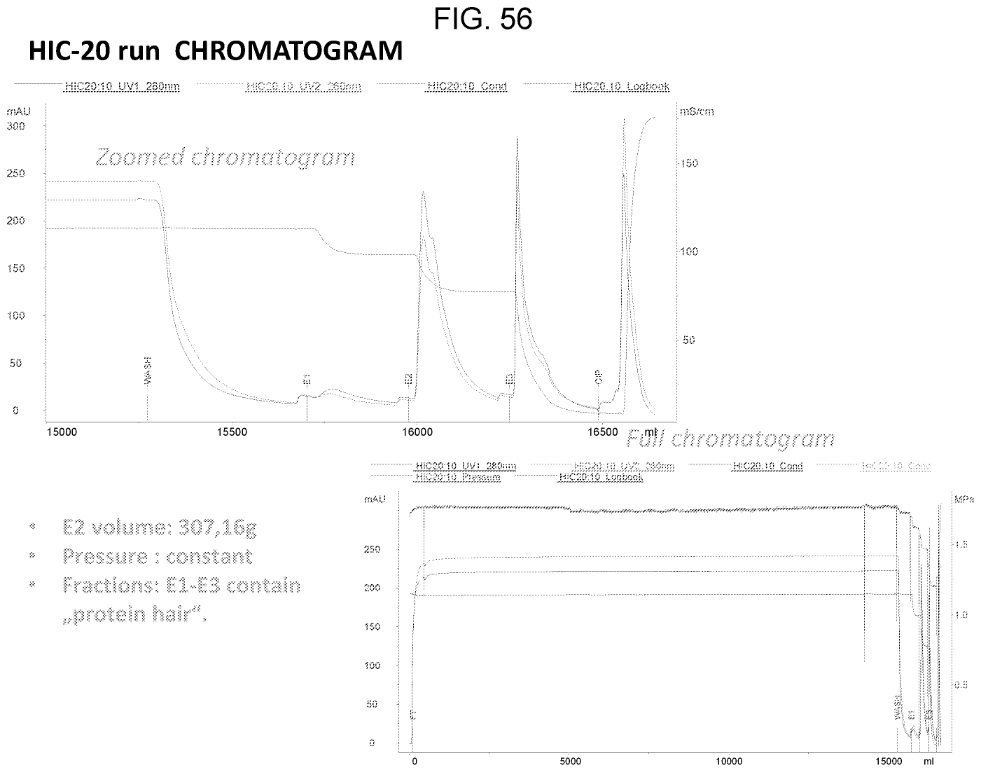

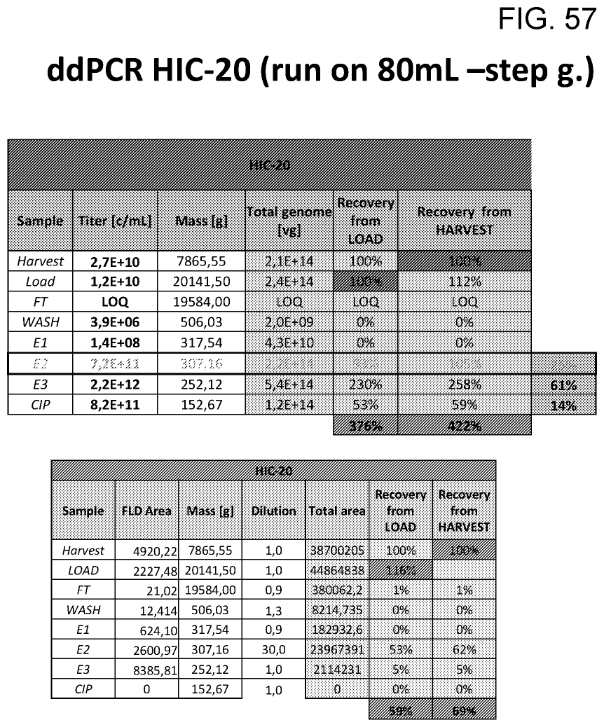

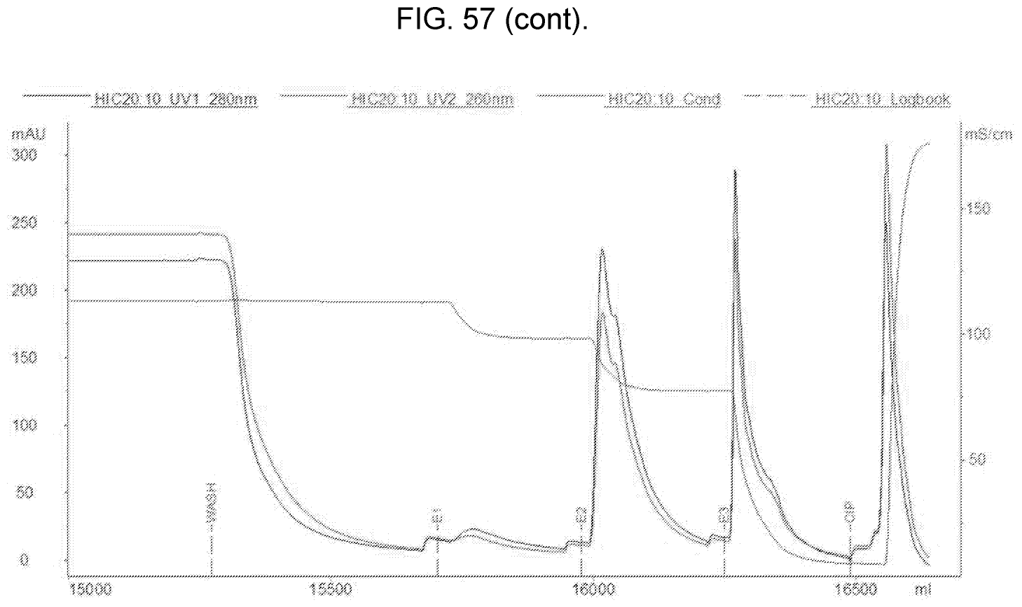

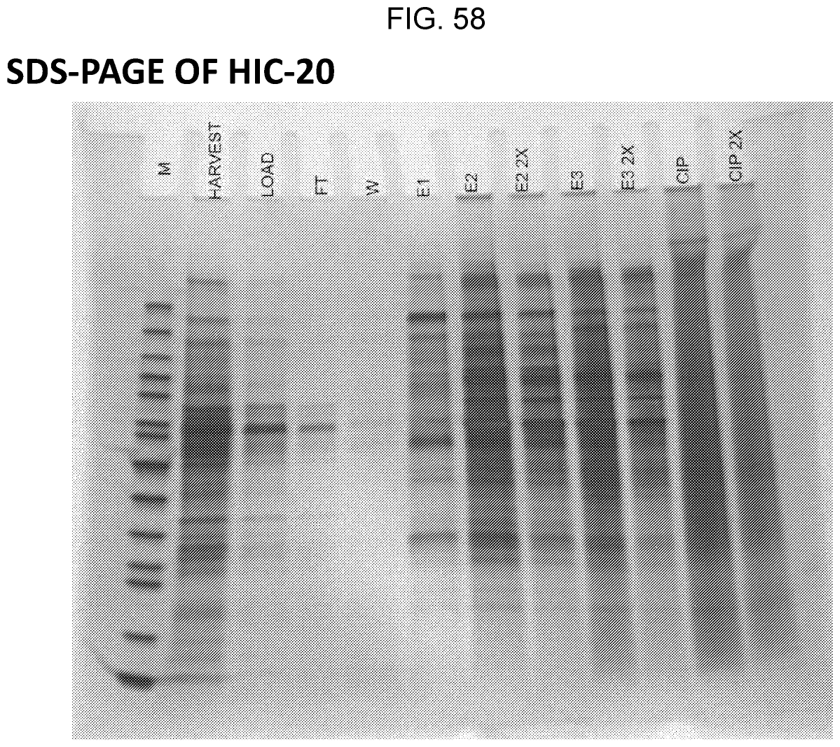

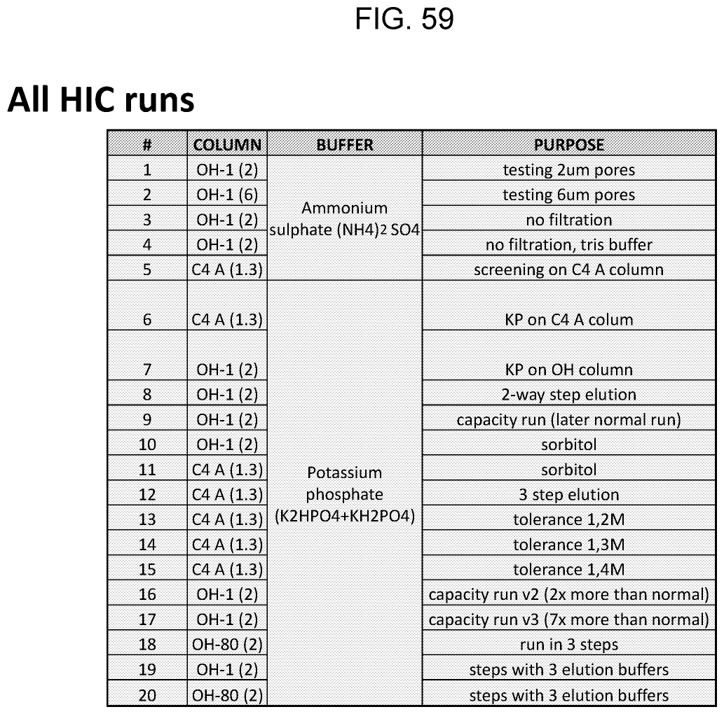

The thawed cells are added to a T-25 flask that contains 4 mL of growth media (DMEM+10% serum) that has been pre-warmed to 37±1° C. The cells are placed in a humidified CO2 incubator that is set to 37° C. and 5% CO2 and left overnight. FIG. 45 shows a flow diagram of the cell thaw step. The parameters and operating conditions to be adhered to during the cell thaw procedure are contained in FIG. 46. FIG. 47 contains details of the key materials and consumables that are required for the cell thaw process.

Cell Expansion

Cells are expanded from the initial T-25 flask through to the 36-layer HYPERStack® units through a series of passages. Cells are grown in humidified incubators set at 37° C. and 5% CO2. Cells are passaged when cell confluency reaches 80%, which is typically every three to five days.

The generic passaging protocol consists of the following:
Remove media from the current cell flask/stack.
Wash the cells using pre-warmed Hanks Balanced Salt Solution (HBSS).
Add pre-warmed cell 1× dissociation solution and swirl the solution to ensure that the cell surface is covered. Remove the excess cell dissociation solution.
Incubate the cell flask/stack for a further 3-5 minutes. Dislodge the cells using careful manual tapping.
Add further growth media to help remove the cells.
Remove the cells into an intermediate storage container.
Combine the cell suspension with new pre-warmed growth media
Seed the new cell flask/stack.
Incubate the cells at 37° C. and 5% CO2.

FIG. 178 provides a high-level summary of the generic passage procedure whilst FIG. 179 details the generic criteria for cell passages. FIG. 180 contains the recommended volumes and seeding densities, related to passages, for each possible cell culture vessel, up to the 36-layer HYPERStack® unit. The recommended minimum warming times are contained in FIG. 176. FIG. 181 contains details of the key materials and consumables that are required for the cell thaw and routine passage steps. Cells are recommended to be sub-cultured for approximately 30 passages. When approaching their useful passage limit, a new vial should be thawed before the old cells are discarded.

Transient Transfection, Benzonase® Addition, Media Release and Harvesting

Following 3 days of growth post seeding, the HYPERStack® media is replaced with fresh DMEM media containing serum and chloroquine (the chloroquine is only required for the calcium phosphate transfection method); this is performed 2-8 hours before transfection. The cells are then transfected with the three production plasmids using either an optimised calcium phosphate or PEIpro® mediated method.

Sufficient DNA plasmid transfection precipitate is prepared in a biological safety cabinet to transfect 5×36-layer HYPERStacks®. The option exists to perform either a calcium phosphate mediated transfection or a transfection that uses PEIpro®. Both methods will be described in this section of the process description. Many of the steps will be common between the two methods, however when there are differences, explicit instructions will be given as to which transfection method is under discussion.

Calcium Phosphate Specific Transfection

Initially a DNA/calcium mix is prepared containing the transgene plasmid, the AV helper plasmid, the capsid plasmid and CaCl₂. After mixing well, the plasmid/CaCl₂ solution is added to an equal volume of 2×HEPES buffered NaHPO4 with concurrent gentle agitation in a disposable process bag to obtain an optimal precipitate. The solution is sat at room temperature for at least 5 minutes and then added to the five HYPERStacks® linked with a manifold. This procedure is repeated four times to complete the transfection of the 20 HYPERStack® units.

PEIpro® Specific Transfection

The transgene plasmid, the AV helper plasmid and the capsid plasmid DNA are diluted in serum-free media and stirred gently. Diluted PEIpro® is added to the DNA solution; all at once. The resulting solution then needs to be gently agitated and left to equilibrate to room temperature. The PEIpro®/DNA complex solution is then added to the five HYPERStacks® linked with a manifold. This procedure is repeated four times to complete the transfection of the 20 HYPERStack® units.

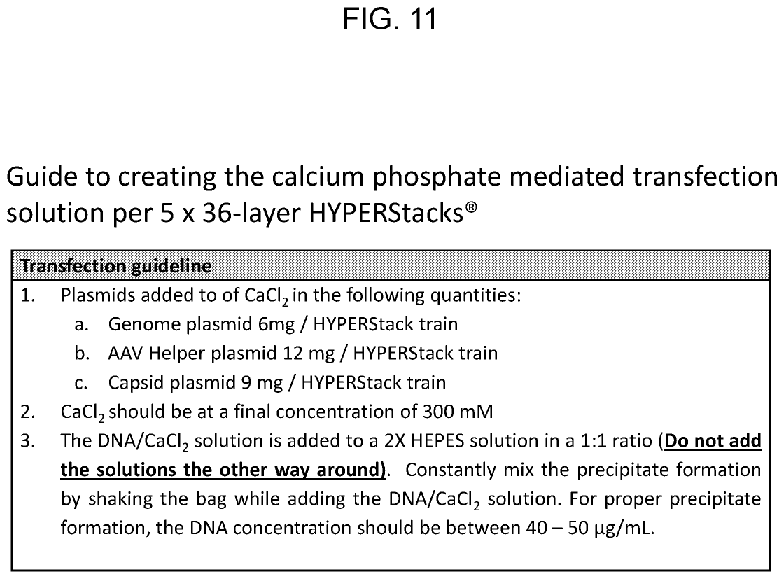

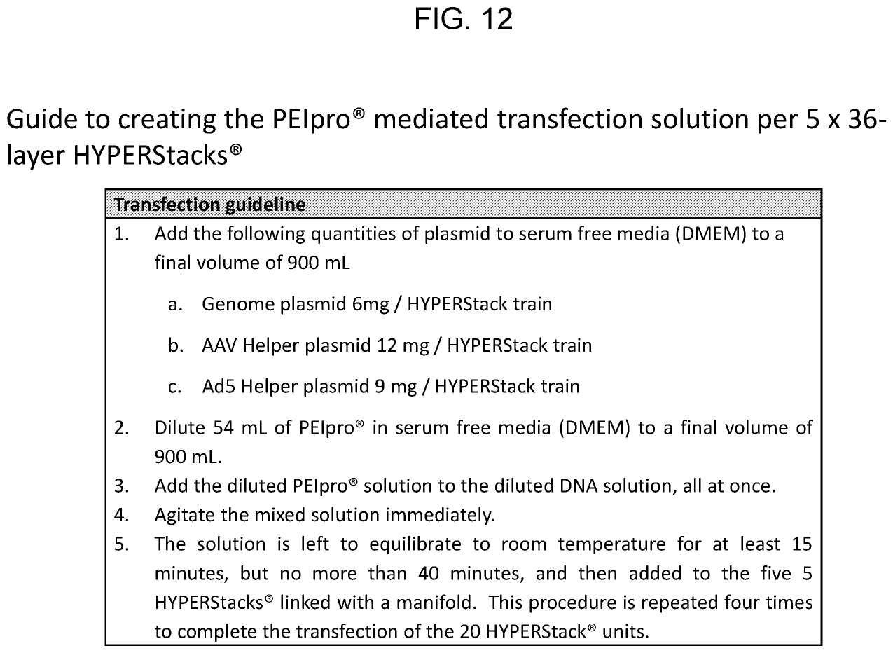

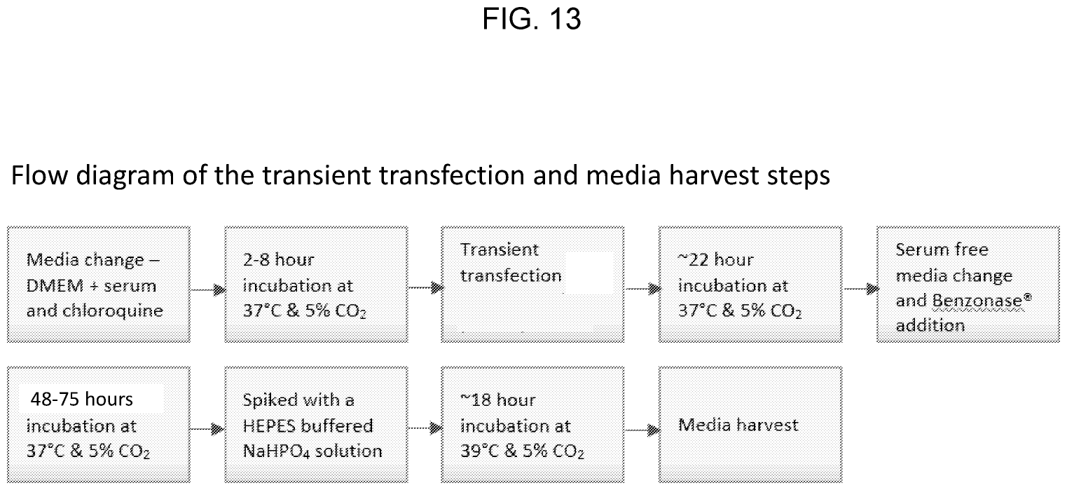

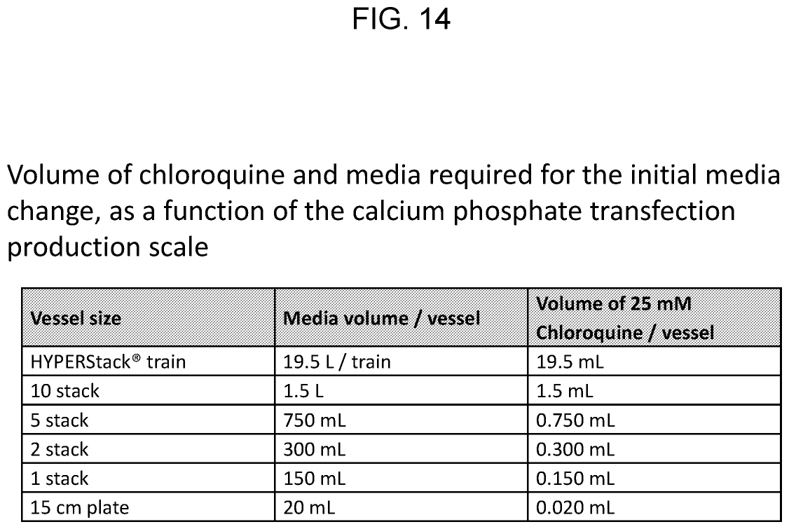

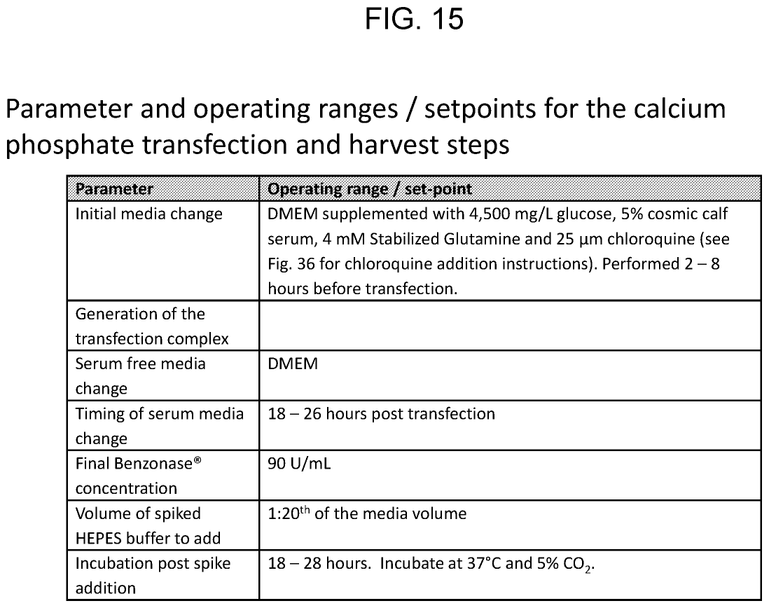

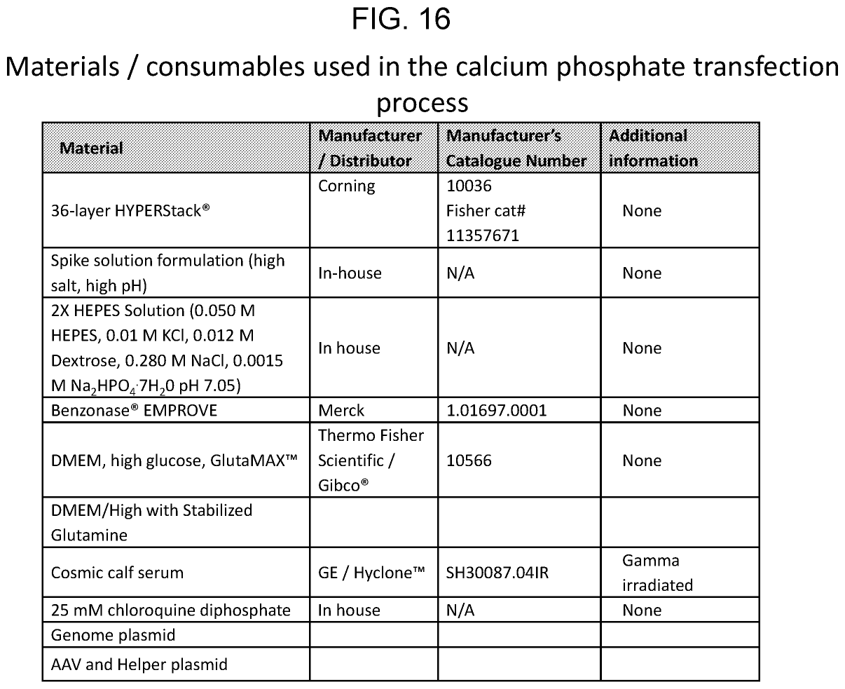

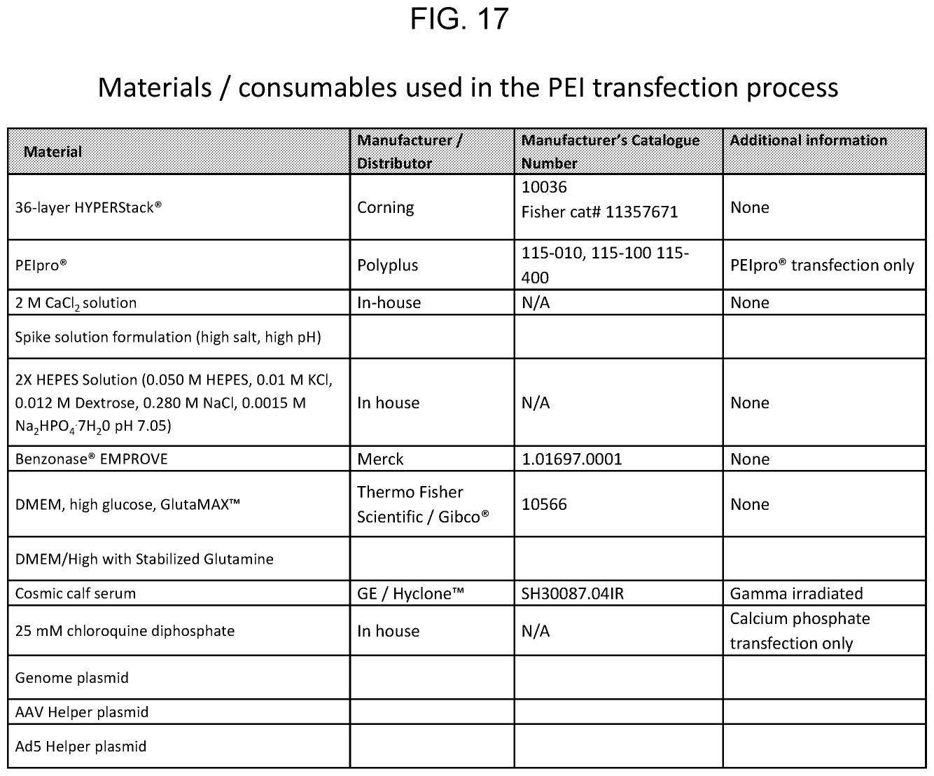

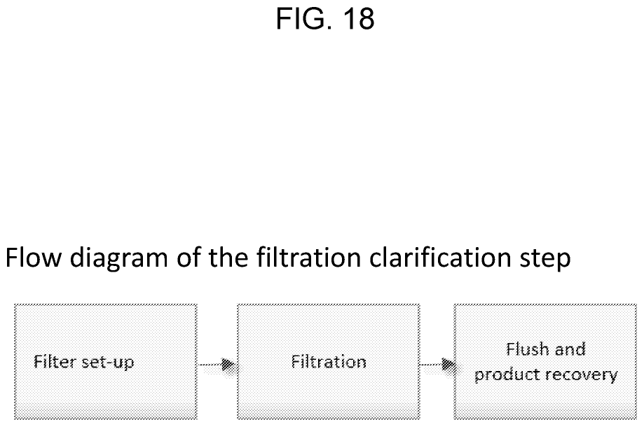

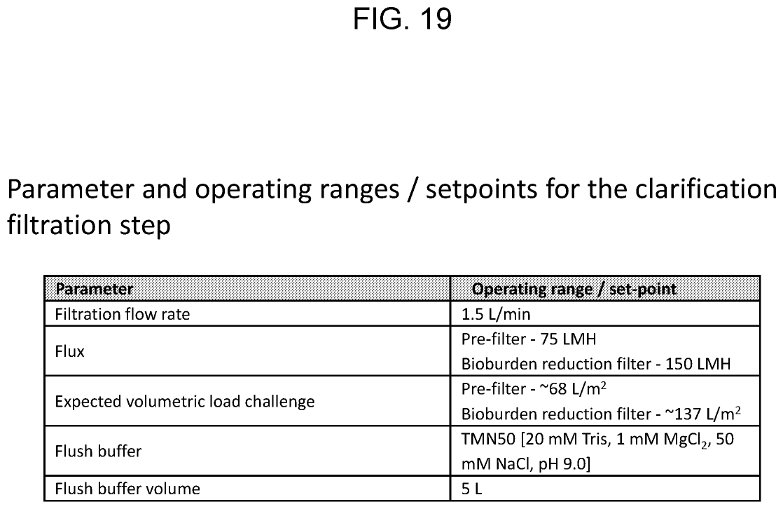

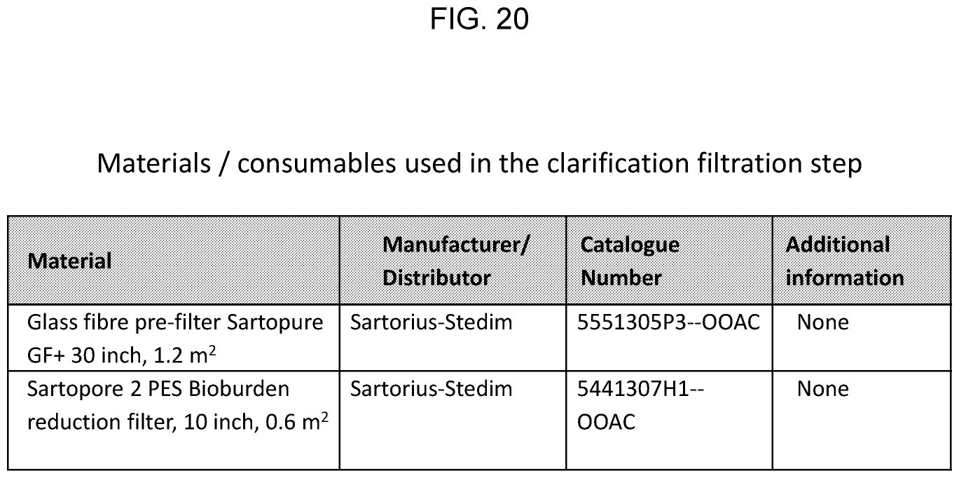

Post transfection, the cells are incubated in an incubator set at 37° C. and 5% CO2. Approximately 22 hours after transfection, the medium is changed using serum-free DMEM. At this time the Benzonase® endonuclease is added to the media, at a concentration of 90 U/mL, to digest free genomic DNA and plasmid DNA present in the media. This step is performed to minimize the amount of residual host cell DNA in the final vector product. The cells are then incubated in an incubator set at 37° C. and 5% CO2 for an additional 69-75 hours. To promote vector release, the 36-layer HYPERStacks® are spiked with a HEPES buffered NaHPO4 solution and incubated for approximately 18 hours in an incubator set at 39° C. and 5% CO2 prior to harvest. The media from each 36-layer HYPERStack® is harvested aseptically using disposable bioprocess bags and pooled into a single volume (˜82 L). FIG. 10 shows a flow diagram of the transient transfection and media harvest steps. FIG. 183 contains the volumes of chloroquine per cell culture unit and FIG. 184 details the operating ranges for the transfection and harvest steps. The specific guidelines for creating the transfection are contained in FIG. 11 and FIG. 12 for the calcium phosphate and PEIpro® transfection methods respectively. The ratio of PEI:DNA ratio is given as a 2:1 ratio in FIG. 12, however it is acceptable to use other ratios, e.g., ratios ranging from 1:1 to 4:1. FIG. 16 contains the details of the key consumables and materials used in the calcium phosphate transfection process. FIG. 17 contains the details of the key consumables and materials used in the PEI transfection process.

Clarification by Filtration

The pooled media containing the recombinant AAV (rAAV) is then clarified through a capsule pre-filter, followed by a sterilising grade capsule filter. A second back-up pre-filter is installed as part of the filtration set up and is to be used if the inlet pressure reaches 10 psi when the first pre-filter is in use. The pre-filter has a pore size of 0.65 μm and is constructed of, glass fibre. The bioburden reduction filter is a 0.2 μm sterilizing grade filter constructed of polyethersulfone (PES). To achieve maximal recovery, after product filtration, filters are blown down aseptically and chased with buffer. FIG. 186 shows a flow diagram of the filtration clarification step. FIG. 187 contains the operating parameters for the filtration clarification unit operation. FIG. 188 shows key materials/consumables used in the clarification filtration step.

Preferred Chemicals for Solution Preparation

Compendial or multi-compendial chemicals are to be used wherever possible. FIG. 208 provides a list of the preferred chemicals and associated grades that have been used in the process.

### Example 5: AAV8-RPGR Manufacturing Process Description—Downstream and Fill and Finish Unit Operations

X-linked retinitis pigmentosa (XLRP) is a very severe form of retinitis pigmentosa (RP), resulting in rapid disease progression and severe retinal dysfunction. The worldwide prevalence of XLRP is approximately 1:30,000 to 1:40,000 (Tee et al., 2016). Patients with XLRP typically experience onset of night blindness in the first decade, followed by reduction of visual field and acuity and progressively severe visual impairment. Most patients are legally blind by the end of the fourth decade.

To date, 3 genes have been mapped to XLRP: RP2; RP3, also known as the RP GTPase regulator (RPGR) gene; and OFD1, which has been identified as a rare cause of XLRP (Webb et al., 2012). Approximately 75% of cases of XLRP are due to RPGR variants, and the worldwide prevalence of XLRP due to RPGR variants is approximately 1:40,000 to 1:53,000 (Pelletier et al., 2007; Shu et al., 2008). RPGR is involved in protein distribution in photoreceptors and plays a role in the transport of photo-transduction components and other outer segment proteins across the connecting cilium (Tee et al., 2016). Essential for photoreceptor viability, the RPGR gene product is localised in the outer segment of rod photoreceptors (Ferrari et al., 2011). Loss of RPGR function in the retina causes the progressive loss of rod and cone vision.

Nightstar Therapeutics is developing AAV8-RPGR as a potential gene therapy medicinal product (GTMP) for the treatment of XLRP due to mutations in RPGR. Replacing the deficient RPGR in XLRP patients with new and viable RPGR is expected to slow or stop retinal degeneration and improve visual function.

This document describes the upstream and primary harvest processes used for the manufacture of AAV8-RPGR product. This document includes all the downstream and primary fill & finish processing steps.

Manufacturing Process Description and Process Controls

Batch Definition

A batch of product defined as a single production campaign consisting of 20 Corning 36-stack HYPERStack® vessels containing plasmid DNA transfected HEK293 cells that produce the AAV8 RPGR biologic product. The cell culture media is harvested and pooled from the 20 Corning 36-HYPERStack® vessels and followed by a single purification process.

Summary of the Downstream Process

After the clarification of the process stream a tangential flow filtration (TFF) step is used to perform a 100 fold volumetric concentration factor of the product followed by diafiltration step into the TMN500T buffer. A 100 kDA modified polyethersulfone (mPES) membrane is employed for this step.

The TFF concentrated media is further purified using a discontinuous iodixanol gradient. This step serves to enrich the preparation for DNA-containing rAAV particles, while removing the bulk of rAAV particles that are devoid of DNA (empty particles) based on the differential buoyant density of these particles. For maximal throughput, the process is completed in two gradient steps.

The iodixanol fraction is further purified on a Sepharose High Performance (SPHP) column which is cation exchange step which captures the positively charged AAV vector whilst other residual impurities and iodixanol are removed from the process stream.

Final vector concentration and diafiltration is achieved using a 100 kDa, TFF mPES membrane. The product is diafiltered into the final formulation buffer (20 mM Tris pH 8.0, 1 mM MgCl₂, 200 mM NaCl). Prior to final formulation, in-process samples are analysed for vector recovery using a qPCR method to determine vector titre and yield of DNase Resistant Particles (DRP). This data is used to estimate the final volume required to achieve the final target titre. The excipient poloxamer 188 is added manually to process stream at a final concentration of 0.001% (v/v). After final formulation, the product is terminally sterile filtered through a 0.22 μm filter to yield the Purified Bulk Drug Substance (PBDS). Filling the PBDS completes the process and yields the Final Drug Product (FDP). Release testing takes place on both the PBDS and FDP.

Manufacturing Flow Diagram

An overview of the downstream and fill and finish steps of the manufacturing process for AAV8-RPGR is illustrated in FIG. 63 along with an overview of the current process controls and QC/analytical tests performed throughout the manufacturing process.

Downstream Manufacturing Process Description

Large Scale Tangential Flow Filtration

The SSS (salt and surfactant solution) is added to the clarified harvest using 1 part SSS buffer to 9 parts clarified media. The addition of the SSS buffer is performed to maintain the solubility of proteins in the clarified media. A 100 kDA mPES hollow fibre membrane is utilised to perform a 100-fold volumetric concentration of the product. The concentration is followed by four dia-filtrations which buffer exchange the product into TMN500T (20 mM Tris, 1 mM MgCl₂, 500 mM NaCl, 0.1% Tween 20). A final concentration takes place after the diafiltration step to reach the target volumetric concentration factor. FIG. 189 provides an overview of the steps of the TFF step. FIG. 190 lists the parameters and associated operating ranges or setpoints which are to be used for the large scale TFF run. FIG. 191 contains the details of the key materials and consumables that are to be used in the large scale tangential flow filtration unit operation.

Additional Comments—Take care not to allow air into the flow loop during the final concentration step as this can initiate frothing of the product.

Initial Iodixanol Concentration

An initial ultra-centrifugation concentration step is performed to reduce the volume that will be processed in the subsequent iodixanol gradient step. The reduction in volume is necessary as the volumetric throughput of the iodixanol gradient separation is limited.

The product from the preceding TFF step is aliquoted into 32 mL volumes and placed into centrifuge tubes. 1×TMNK buffer can be used to top up the last centrifuge tube in the likely event that it is less than 32 mL. A single layer of 3 mL of 57% iodixanol solution is underlaid into each product containing tubes. The centrifuge tubes are loaded into a centrifuge and spun at 65,000 rpm for 30 minutes utilising a temperature of 4° C. The centrifugation is repeated as necessary to process the entire product stream. The entire 57% iodixanol band is harvested alongside 1 mL of the 57% interface. The harvested product is then diluted in a 1×TMNK buffer. FIG. 192 provides an illustrative summary of the iodixanol concentration step. FIG. 193 details the parameters and set points to be employed for the centrifugation concentration step. Key materials and consumables to be used in the centrifugation concentration step are contained in FIG. 194.

Additional Comments
Avoid generating bubbles or foaming when transferring the ‘Lg TFF Concentrate’ Pool sample into the bottom of the ultracentrifuge tube.
Add the 57% underlay slowly to avoid unwanted mixing of the phases.
When harvesting, puncture the top of the centrifuge tube to stop a vacuum being formed when collecting the product

Iodixanol Gradient Purification

The centrifuged concentrated media is further purified using a discontinuous iodixanol gradient. This step serves to enrich the preparation for DNA-containing rAAV particles, while removing the bulk of rAAV particles that are devoid of DNA (empty particles) based on the differential buoyant density of these particles in the iodixanol gradient medium following ultracentrifugation. The discontinuous gradient is formed of 25, 40 and 57% iodixanol phases. After centrifugation the DNA enriched vector is harvested from just below the 40/57% interface. The bulk of the empty particles are contained in the 25/40% interface. The harvested pooled vector is diluted in 1×TMNK buffer to prevent aggregation of the AAV vector. FIG. 195 provides a graphical overview of the steps required to complete the iodixanol gradient purification step. FIG. 196 lists the parameters and associated operating ranges or setpoints which are to be used for the iodixanol gradient centrifugation step whilst FIG. 197 contains the associated key materials and consumables.

Additional Comments
Avoid generating bubbles or foaming when transferring the adding the iodixanol bands.
Add iodixanol solutions slowly to avoid unwanted mixing of the phases.
When harvesting, puncture the top of the centrifuge tube to stop a vacuum being formed when collecting the product

Cation Exchange Chromatography

The iodixanol harvest fraction is purified over a cation exchange (CEX) chromatography column which serves to remove residual contaminants, including iodixanol.

The iodixanol pool is firstly diluted 7-fold using a dilution buffer (6:1 ratio—dilution buffer to iodixanol pool). This is then followed by a 2-fold dilution using WFI (1:1 ratio—WFI to diluted iodixanol pool). The dilution of the iodixanol pool is necessary to allow the vector to bind to the cation exchange column by reducing the conductivity and lowering the pH of the sample.

The 14-fold, fully diluted, iodixanol pool becomes the load for the cation exchange step which utilises an SP Sepharose™ HP resin. The binding of the vector takes place in a low conductivity and low pH citrate based buffer and the elution is achieved by the use of a high salt buffer. The vector containing elution peak is then diluted with an AMPD buffer (1:9 ratio—AMPD buffer to CEX eluate) before it is stored at 2-8° C. before subsequent processing. An overview of the CEX unit operation is illustrated in FIG. 198 whereas the full operating parameters for the cation exchange chromatography step are contained in FIG. 199 and FIG. 200. The key materials and consumables required for the successful execution of the CEX step are listed in FIG. 201 with their associated details.

Small Scale Tangential Flow Filtration and Excipient Addition

Final vector formulation is achieved using a 100 kDa, TFF mPES membrane. Prior to final formulation, in-process samples are analysed for vector recovery using a qPCR method to determine vector titre and yield of DNase Resistant Particles (DRP). This data is used to estimate the final volume required to achieve the final target titre. The product is diafiltered into the final formulation buffer (20 mM Tris pH 8.0, 1 mM MgCl2, 200 mM NaCl). The excipient poloxamer 188 is added manually to a final concentration of 0.001% (v/v). FIG. 202 provides a graphical overview of the steps required to complete the small scale tangential flow filtration step. FIG. 203 lists the parameters and associated operating ranges or setpoints which are to be used for the small scale TFF run. FIG. 204 contains the details of the key materials and consumables that are to be used in the small scale tangential flow filtration unit operation.

Sterile Filtration and Vialling

After final formulation, the final titre is determined and then the product is terminally sterile filtered through a 0.22 μm filter to yield the Purified Bulk Drug Substance (PBDS). The PBDS is filled into sterile tubes, upon which the product becomes the Final Drug Product (FDP). The FDP is inspected before it is stored at <−60° C. FIG. 204 shows a flow chart of the sterile filtration and filling unit operations. FIG. 205 lists the parameters and associated operating ranges or setpoints which are to be used for the sterile filtration and filling operations. FIG. 206 contains the details of the key materials and consumables that are to be used in the sterile filtration and filling steps.

In-Process Hold Conditions

FIG. 207 contains the details of the hold times at in-process points that have been used during the manufacture of the AAV8-RPGR product. As more information becomes available, the in-process hold times will be refined to reflect the latest data.

Preferred Chemicals for Solution Preparation

Compendial or multi-compendial chemicals are to be used wherever possible. FIG. 208 provides a list of the preferred chemicals and associated grades that have been used in the process.

### Example 6: Downstream Process for AAV8-RPGR Production

The aim of the project was to develop an industrial chromatographic downstream process (DSP) for rAAV8 RPGR late stage clinical and commercial program. The project included all developed steps—capture, intermediate polishing and separation of empty-full (E/F) AAV8 capsids using Macro-porous OH, SO3 and QA columns, and a tangent flow filtration (TFF) following client's protocol. Development was based on clarified harvest material where calcium phosphate was used as a transfecting agent.

Materials and Methods

Sample

Sample was formulated in clarified DMEM medium. Two different experimental runs were conducted on different dates, Experiment A and Experiment B. FIG. 210 contains sample details of Experiment A. FIG. 234 contains sample details of Experiment B.

FPLC Systems (Preparative Runs)

### FPLC 2:

GE Healthcare Akta Explorer 100, UV flow cell 2 mm
0.75 mm I.D. capillaries (used with 8 and 80 mL column)
Sample loading: loading via system pump
Detection: UV 280 nm, UV 260 nm, conductivity, pH

HPLC Systems (Analytical Runs)

### HPLC 1:

PATfix™, 10 mL pump heads, 0.25 mm I.D. capillaries
Sample loading: 500 μL sample loop
Detection: UV 280 nm, UV 260 nm, fluorescence 280/348 (FLU, FLD), conductivity, MALS
Flow rate: 1-2 mL/min

Monolith Stationary Phases

Analytics runs (3 columns):
Macro-porous Adeno-0.1
Macro-porous SO3-0.1
Macro-porous AAV empty/full-0.1
   Preparative runs (3 columns):
Macro-porous OH-80
Macro-porous SO3-8
Macro-porous QA-8

Buffers

Buffers were prepared in fresh purified water and filtered through 0.22 μm filters. FIG. 211 shows buffers used for preparative and analytical runs for Experiment A. FIG. 235 shows buffers used for preparative and analytical runs for Experiment B.

Chromotographic Methods

Preparative Runs:
HIC step—HIC purification step was performed as specified in the downstream processing SOP. FIG. 212 shows SOP step gradients with dedicated buffers for Experiment A. FIG. 236 shows SOP step gradients with dedicated buffers for Experiment B.
CEX Step—CEX purification step was performed as specified in the downstream processing SOP. FIG. 213 shows SOP step gradients with dedicated buffers for Experiment A. FIG. 237 shows SOP step gradients with dedicated buffers for Experiment B.
AEX Step—AEX purification step was performed in the downstream processing SOP. FIG. 214 shows SOP linear gradient from 0 to 100% mobile phase B in 60 column volumes (CVs) and then step to 100% MPC for 10 CVs for Experiment A. FIG. 238 shows SOP linear gradient from 0 to 100% mobile phase B in 60 column volumes (CVs) and then step to 100% MPC for 10 CVs for Experiment B.

Analytic Runs:
Partial Separation—linear gradient from 0 to 35% mobile phase B in 50 CV, then from 35 to 100% in 5 CV. Partial Separation method was performed as specified in the analytical HPLC SOP.
Total—linear gradient from 0 to 100% mobile phase B in 50 CV. Total method was performed as specified in the analytical HPLC SOP.
Empty/Full—Linear gradient from 0 to 40% mobile phase B in 50 column volumes (CV), then from 40 to 100% in 10 CV.

Total Protein Assay

Samples were tested for total protein concentration following two assays. Either BCA Pierce method or Bradford method was used depending on buffer composition. Manufacturer protocol was followed.

Total DNA Assay

For total DNA quantification in samples a Quant-IT™ PicoGreen® assay was used. Manufacturer protocol was followed.

SDS-PAGE

SDS-PAGE was carried out with a Mini-Protean II electrophoresis Cell (Bio-Rad) using 4-20% gradient gels under reducing conditions according to the manufacturer's instructions (Bio-Rad). The gels were run at 200 V for 35 min using a discontinuous Tris-glycine buffering system. Protein bands were visualized by Plus one Silver staining reagent (GE Healthcare). A 10-200 kDa molecular weight standard was used (Fermentas Life Sciences). Each time 20 ul of sample in appropriate dilution, was loaded to the well.

TEM

Samples were prepared for examination with TEM using negative staining method. Thawed samples were mixed gently and applied on freshly glow-discharged copper grids (400 mesh, formvar-carbon coated) for 5 minutes, washed and stained with 1 droplet of 1% (w/v) water solution of uranyl acetate.

The grids were observed with transmission electron microscope Philips CM 100 (FEI, The Netherlands), operating at 80 kV. At least 10 grid squares were examined thoroughly and several micrographs (camera ORIUS SC 200, Gatan, Inc.) were taken to evaluate the ratio between full and empty particles. Micrographs were taken coincidentally at different places on the grid.

ddPCR

Samples (and control) were DNAze treated and diluted in three points in duplicates (6 reactions for each sample). Reaction mix: ddPCR Supermix for Probes (no dUTP). Reaction volume: 20 uL, DNA volume 5 uL, Droplet volume 0.000739. Equipment used: Bio-Rad QX100™ Droplet Digital™ PCR System, Bio-Rad QX200™ AutoDG™ Droplet Digital™ PCR System, Fluidigm Biomark HD. Primers and probes used based on clients recommendation.

Capture Step on Hydrophobic Interaction Chromatography (HIC) Using Macro-Porous OH Columns HPLC Analytical Methods

Preparative Run

Clarified harvest material (8 L divided in 1 L bottles) was thawed overnight at room temperature. Next day it was pooled and diluted 1:1 (8 L harvest+8 L buffer) with dilution buffer using peristaltic pump at speed 400 mL/min. Loading to the column using system pump at 1 CV/min. Tech transfer run was the twenty-fifth (25) run for HIC conditions (HIC-25) for Experiment A. FIG. 215 details the preparative run conditions for Experiment A. FIG. 216 shows exemplary chromatograms from run HIC-25 for Experiment A. Tech transfer run was the twenty-sixth (26) run for HIC conditions (HIC-26) for Experiment B. FIG. 239 details the preparative run conditions for Experiment B. FIG. 240 shows exemplary chromatograms from run HIC-26 for Experiment B.

HPLC Total Analytics

Total particle method was used on HPLC for determination of chromatographic recovery. Fractions were desalted using Amicon Ultra 0.5. Main elution was further diluted 10× prior injection. FIG. 217 shows exemplary chromatograms based on HPLC analysis for Experiment A. From FIG. 217 we can confirm that all AAV binds to the column, and elutes in fractions W2, E1 and W3. When observing picture J (overlay) we can see that both fractions W2 and W3 have other protein impurities present compared to main E1 elution. We also have to account that faction E1 is 10-fold diluted compared to other two, so loss of vector in fractions surrounding eluate is negligible. Areas of peaks were compared to load and harvest area peaks, to determine recoveries.

FIG. 241 shows exemplary chromatograms based on HPLC analysis for Experiment B. From FIG. 241 we can confirm that all AAV binds to the column, and elutes in fractions W2, E1 and W3. When observing picture J (overlay) we can see that both fractions W2 and W3 have other protein impurities present compared to main E1 elution. We also have to account that faction E1 is 10-fold diluted compared to other two, so loss of vector in fractions surrounding eluate is negligible. Areas of peaks were compared to load and harvest area peaks, to determine recoveries.

Recovery of Preparative Run

Recoveries for capture step HIC-OH comparing to starting clarified harvest material are 102% and 68% for ddPCR and HPLC Total analytics, respectively. The discrepancy between two methods is mainly caused by high salt concentration in sample, moreover the mass balances are not 100% in both cases, so normalization of two would result in more accurate results with average 80-90% recovery of AAV in main fraction. FIG. 218 details recoveries of HIC-25 run based on ddPCR and HPLC total analytics from Experiment A. FIG. 242 details recoveries of HIC-26 run based on ddPCR and HPLC total analytics from Experiment B. FIG. 219 is a representative SDS-PAGE result for HIC-25 run for Experiment A. M—ladder. Fractions E1, W3 and CIP are 5-fold, 5-fold and 2-fold diluted, respectively. Main fraction is E1. VP1-VP3 proteins are marked by red rectangle.

SDS-PAGE

All fractions were desalted first and then loaded to the gel either neat or diluted under reducing conditions. FIG. 218 shows concentration of AAV and successful capture is achieved from clarified harvest material for Experiment A. Main elution after HIC step has many protein impurities which are removed by next chromatography step CEX-SO3. SDS-PAGE results from HIC-25 run. FIG. 242 shows concentration of AAV and successful capture is achieved from clarified harvest material for Experiment B. Main elution after HIC step has many protein impurities which are removed by next chromatography step CEX-SO3. SDS-PAGE results from HIC-26 run.

Intermediate Polishing on Cation Exchange Chromatography (CEX) Using Macro-Porous SO3 Column

Preparative Run

Entire elution (E1) from HIC-OH was prepared to match binding conditions and loaded to CEX-SO3 column. Tech transfer run was a sixteenth (16) run for CEX conditions (SO3-16) for Experiment A. FIG. 220 details the preparative run conditions for Experiment A. FIG. 221 shows an exemplary chromatogram from run SO3-16 for Experiment A.

Tech transfer run was a seventeenth (17) run for CEX conditions (SO3-17) for Experiment B. FIG. 244 details the preparative run conditions for Experiment B. FIG. 245 shows an exemplary chromatogram from run SO3-17 for Experiment B.

HPLC Total Analytics

Total particle method was used on HPLC for determination of chromatographic recovery. Fractions were 100-fold (E1) or 2.5-fold (other fractions) diluted prior injection. FIG. 222 shows exemplary chromatograms based on HPLC analytics-Total method for SO3-16 for Experiment A. From FIG. 222 we can confirm that all AAV binds to the column, and elutes in fractions E1 and W3. We have to account that faction E1 is 100-fold diluted compared and W3 is 5-fold diluted so loss of vector in W3 fraction negligible. Areas of peaks were compared to load and initial HIC-25 E1 material, to determine recoveries. FIG. 223 details recoveries based on ddPCR and HPLC Total analytics for preparative run SO3-16 for Experiment A. Recoveries for intermediate polishing step CEX-SO3 comparing to starting HIC-28 E1 material are 99% and 87% for ddPCR and HPLC Total analytics, respectively. The discrepancy between two methods is minor. In case of HPLC analytics, mass balance is not 100%.

FIG. 246 shows exemplary chromatograms based on HPLC analytics-Total method for SO3-17 for Experiment B. From FIG. 246 we can confirm that all AAV binds to the column, and elutes in fractions E1 and W3. We have to account that faction E1 is 100-fold diluted compared and W3 is 5-fold diluted so loss of vector in W3 fraction negligible. Areas of peaks were compared to load and initial HIC-26 E1 material, to determine recoveries. FIG. 247 details recoveries based on ddPCR and HPLC Total analytics for preparative run SO3-17 for Experiment B. Recoveries for intermediate polishing step CEX-SO3 comparing to starting HIC-28 E1 material are 99% and 87% for ddPCR and HPLC Total analytics, respectively. The discrepancy between two methods is minor. In case of HPLC analytics, mass balance is not 100%.

SDS-PAGE

All fractions were loaded to the gel either neat or diluted under reducing conditions. FIG. 224 shows SDS-PAGE results for SO3-16 run for Experiment A. FIG. 224 portrays further concentration of AAV, since 10-fold lower column size was used from HIC to CEX step. Main elution after HIC step has other protein impurities present apart from AAV viral bands. In wash 3 there is a small portion of AAV band visible. The majority of host cell proteins are removed by strip with CIP.

FIG. 248 shows SDS-PAGE results for SO3-17 run for Experiment B. FIG. 248 portrays further concentration of AAV, since 10-fold lower column size was used from HIC to CEX step. Main elution after HIC step has other protein impurities present apart from AAV viral bands. In wash 3 there is a small portion of AAV band visible. The majority of host cell proteins are removed by strip with CIP.

Empty and Full AAV Capsids Separation on Anion Exchange Chromatography (AEX) Using Macro-Porous QA Column

Preparative Run

Entire elution (E1) from SO3-16 was diluted to match binding conditions and loaded to AEX-QA column for Experiment A. Tech transfer run was a fourteenth (14) run for AEX conditions (QA-14). FIG. 225 details the preparative run conditions for Experiment A. FIG. 226 shows an exemplary chromatogram from run QA-14 from Experiment A.

Entire elution (E1) from SO3-17 was diluted to match binding conditions and loaded to AEX-QA column for Experiment B. Tech transfer run was a fifteenth (15) run for AEX conditions (QA-15). FIG. 249 details the preparative run conditions for Experiment B. FIG. 250 shows an exemplary chromatogram from run QA-15 from Experiment B.

HPLC Empty-Full Analysis

Empty-full method was used on HPLC for determination of chromatographic recovery and purity (ratio of E/F capsids). Fractions were diluted prior injection. FIG. 227 shows exemplary chromatograms based on HPLC analytics—Empty-full method for QA-14 from Experiment A. From FIG. 227 we can confirm that all AAV binds to the column since no peaks are visible in FT+W fraction. Due to slight difference in charge empty capsid start to elute first (E2) which are followed by full capsids found in E3. The difference in A260/A280 ratios confirms that AAV are pure in empty or full capsids. Values of 0.6 in A260/A280 ratios correspond to empty capsids, with predominantly protein composition, where full capsids which have DNA insert give a value of 1.3 and higher depending on the purity. Fraction E4 is collected separately since lower purity is obtained due to empty capsid contamination from next eluting peak. E5 fraction has predominately empty, aggregated and damaged capsids (two peaks), there is no AAV elution in E6 fraction. Areas of peaks were compared to load and initial SO3-16 E1 material, to determine recoveries and purity.

FIG. 251 shows exemplary chromatograms based on HPLC analytics—Empty-full method for QA-15 from Experiment B. From FIG. 251 we can confirm that all AAV binds to the column since no peaks are visible in FT+W fraction. Due to slight difference in charge empty capsid start to elute first (E2) which are followed by full capsids found in E3. The difference in A260/A280 ratios confirms that AAV are pure in empty or full capsids. Values of 0.6 in A260/A280 ratios correspond to empty capsids, with predominantly protein composition, where full capsids which have DNA insert give a value of 1.3 and higher depending on the purity. Fraction E4 is collected separately since lower purity is obtained due to empty capsid contamination from next eluting peak. E5 fraction has predominately empty, aggregated and damaged capsids (two peaks), there is no AAV elution in E6 fraction. Areas of peaks were compared to load and initial SO3-17 E1 material, to determine recoveries and purity.

Tangent Flow Filtration

Concentration and buffer exchange was achieved by implementation of TFF on QA-14 E3 sample for Experiment A. End volume of sample was 25 mL (10 mL sample+15 mL system hold-up volume). FIG. 228 details the tangent flow filtration conditions for Experiment A.

Concentration and buffer exchange was achieved by implementation of TFF on QA-15 E3 sample for Experiment B. End volume of sample was 35 mL (10 mL sample+25 mL system hold-up volume). FIG. 252 details the tangent flow filtration conditions for Experiment B.

Recovery of Preparative Run

Recoveries for full capsid enrichment step (empty and full separation) step AEX-QA comparing to starting SO3-16 E1 material are 73% and 67% for ddPCR and HPLC Total analytics, respectively, for Experiment A. The discrepancy between two methods is minor. In case of HPLC analytics, and ddPCR mass balance is not 100%. For HPLC E/F analytics only A260 and A280 areas are accounted since fluorescence gives lower response of full AAV capsid recovery due to DNA (insert) quenching FLD signal. Approximately 60-70% recovery is obtained after TFF, meaning the entire downstream yield is 43% or 73% if comparing QA eluate to clarified harvest material. FIG. 229 details recoveries based on ddPCR and HPLC E/F analytics for preparative run QA-14 TFF and total DSP yield for Experiment A.

Recoveries for full capsid enrichment step (empty and full separation) step AEX-QA comparing to starting SO3-17 E1 material are 62% and 64% for ddPCR and HPLC Total analytics, respectively, for Experiment B. The discrepancy between two methods is minor. In case of HPLC analytics, and ddPCR mass balance is not 100%. For HPLC E/F analytics only A260 and A280 areas are accounted since fluorescence gives lower response of full AAV capsid recovery due to DNA (insert) quenching FLD signal. Approximately 70-80% recovery is obtained after TFF, meaning the entire downstream yield is 55% or 82% if comparing QA eluate to clarified harvest material. FIG. 253 details recoveries based on ddPCR and HPLC E/F analytics for preparative run QA-15 TFF and total DSP yield for Experiment B.

Purity (Ration Between Empty and Full AAV Capsids)

FIG. 230 details the purity of both empty and full AAV capsids based on HPLC E/F analytics for Experiment A. FIG. 230 indicates that purity (percentage of full capsids) of main E3 fraction is 87% if FLD is taken in account. Since extinction coefficients for both absorbencies are not known, we cannot rely on their signal; this makes FLD the most reliable value. The ratio drastically changes in base of main peak elution (fraction E4) where ratio is only 55%. The reason for collection of only 3.5 CV (approximately 80% peak) is achieving higher purity in E3 and only a minor loss of vector (E4) (7%).

FIG. 254 details the purity of both empty and full AAV capsids based on HPLC E/F analytics for Experiment B. FIG. 254 indicates that purity (percentage of full capsids) of main E3 fraction is 90% if both MALS and FLD are taken in account. Since extinction coefficients for both absorbencies are not known, we cannot rely on their signal; this makes MALS the most reliable detector, since it measures the diameter of the particle. Next in line is FLD detector regarding the accuracy. The ratio drastically changes in base of main peak elution (fraction E4) where ratio is only 60-70%. The reason for collection of only 3.5 CV (approximately 80% peak) is achieving higher purity in E3 and only a minor loss of vector (E4) (6%).

For Experiment A, purity was additionally tested by TEM, for E2 (empty capsids) and E3 (full capsids) however for full capsids a different stage—QA-14 E3 sample after TFF was evaluated. Sample TFF AAV8-RPGR FULLS contained different kind of impurities in contrast to sample QA-14 E2, which contained only small aggregates of damaged particles. Ratio between full and empty/damaged viruses were similar in both samples, 62% in sample TFF AAV8-RPGR FULLS and 65% in sample QA-14 E2. Relatively high percentages represented unclassified particles. Viruses from this group were not electron lucent on the whole surface, but displayed just electron dense spot on the surface. Such viruses could be not completely full, not correctly formed or damaged. FIG. 231 details the ratio of full and empty AAVs evaluated by TEM for Experiment A. FIG. 232 shows a QA-14 E3 fraction after TFF evaluated by TEM, QA-14 E2 fraction for Experiment A.

Filamentous impurities were found only in sample after TFF, which was later confirmed that derived from TFF that was not properly sanitized. The large portion of full capsids found in empty peak is explained by fraction collection approach, where E2 fraction is prolonged until absorbance crossing where full particles are already eluting and therefore contaminating the empty fraction E2.

For Experiment B, purity was additionally tested by TEM, for QA-15 E3 (full capsids) and sample after TFF (TT BB RPGR-FULLS). Samples TT BB AAV8-RPGR FULLS and QA-15 E3 contained only small aggregates of damaged particles. In both sample some aggregates included also structures usually called discs and most probably represented proteins. In both samples full particles prevailed, but at the time of grid examination we noticed difference in sample QA-15 E3, between non-diluted and diluted samples. We counted and calculated the particles separately for diluted and non-diluted samples. We propose that only calculations from non-diluted samples are taken into account. Sample TT BB AAV8-RPGR FULLS contained 76% of full particles and sample QA-15 E3 contained 84% of full particles. FIG. 255 details the ratio of full and empty AAVs evaluated by TEM for Experiment B. FIG. 256 shows a QA-15 E3 fraction after TFF evaluated by TEM, QA-14 E2 fraction for Experiment B.

SDS-PAGE

All fractions were loaded to the gel either neat or diluted under reducing conditions. FIG. 233 shows SDS-PAGE results for QA-14 run from Experiment A. FIG. 233 portrays that all fractions from E2 to E6 contain AAV. The protein band above 200 kDa mark present in E3 and E4 fractions, corresponds to DNA insert found only in full capsids indicating only those two fraction contain full capsids which complements HPLC E/F analytics results. Other protein impurities are found in E3 fraction aside VP1-VP3. Those impurities are partially removed by TFF (AAV8 FULLS) but other proteins are still present as confirmed also by TEM. Additional protein bands present due to inadequate sanitization of TFF system.

FIG. 257 shows SDS-PAGE results for QA-15 run from Experiment B. FIG. 257 portrays that all fractions from E2 to E6 contain AAV. The protein band above 200 kDa mark present in E3 and E4 fractions, corresponds to DNA insert found only in full capsids indicating only those two fraction contain full capsids which complements HPLC E/F analytics results. Other protein impurities are found in E3 fraction aside VP1-VP3. Those impurities are partially removed by TFF (AAV8 FULLS).

HPLC Analytics—Partial Separation Method

FIG. 258 shows an exemplary chromatogram using the Partial Separation method for Experiment B. From FIG. 258 we can observe the majority of impurities are removed by HIC step (picture A). Sample is not pure enough to achieve separation of empty and full capsid, so additional polishing is performed on CEX-SO3. The eluate from this stage is mainly pure and highly concentrated, but still consists of both empty and full capsids. Last AEX-QA step separates the two capsids, and therefore isolates and enriches full capsids. By comparing harvest material to QA main fraction, one can identify the AAV peak from starting material.

Conclusions

A seamless downstream purification run was performed using clarified harvest as starting material. Capture and concentration of AAV was achieved by HIC-OH step, where proteins were found in flow through and AAV was bound to the column. Protein impurities were removed in either W2 or W3 fractions.

Large portion of protein impurities were still present in main elution fraction (E1) after HIC step. The majority of protein impurities were removed by the intermediate polishing step using CEX-SO3 column, where additional concentration of AAV was achieved by implementation of a 10-fold lower column scale. The percentage of full capsid at this stage was approximately 55% for Experiment A and 34% for Experiment B, so full particle enrichment using AEX-QA was performed.

After separating full capsid from empty capsids a buffer exchange in to formulation buffer was performed using TFF. The entire downstream process yield from clarified harvest to completion of TFF was 43% and 73% from clarified harvest to completion of QA full particle enrichment step for Experiment A. The entire downstream process yield from clarified harvest to completion of TFF was 55% and 82% from clarified harvest to completion of QA full particle enrichment step for Experiment B.

### Example 7: ABCA4 Purification Process Compatibility Study

The disclosure provides an industrial chromatographic downstream process (DSP) for Stargardt (ABCA4) late stage clinical and commercial program. The project included all developed steps—capture, intermediate polishing and separation of empty-full (E/F) AAV8 capsids using Macro-porous OH, SO3 and QA columns, and a tangent flow filtration (TFF).

A compatability study was performed for ABACA4 vector wherein a proxy vector was used. The proxy vector has the same capsid (AAV8/Y733F) as the ABCA4 vector. The capsid is the determining factor for the behavior of the vector across the HIC and SO3 steps. FIG. 259 details the HIC (OH) chromatography conditions. FIG. 260 shows an exemplary HIC (OH) chromatogram and vector recovery analysis as measured by HPLC total particle analytics.

FIG. 261 details the CEX (SO3) chromatography conditions. FIG. 262 shows CEX (SO3) exemplary chromatograms and vector recovery analysis.

The packaged genome was a construct comprising a Bestrophin-1 gene (which is smaller than the ABCA4 gene and does not require a dual vector, allowing for proof of concept studies on the vector itself). The packaged genome has an effect on the behavior over the QA step. This step employs a linear gradient, therefore it was anticipated that there would be no changes to the operating conditions for the QA step when the ABCA4 transgene is used. FIG. 263 details AEX (QA) chromatography conditions. FIG. 264 shows an exemplary chromatogram and vector recovery analysis of empty and full particles in the QA fraction.

Optimal representation of purity (E/F) ratio is given by FLD and MALS detectors. Enrichment from approximately 55% to 94% of full AAV particles is achieved by the QA step. FIG. 265 details purity of (Full:Empty) particles based on HPLC analytics. FIG. 266 shows purity (Full:Empty) based on TEM. FIG. 267 shows purity by SDS-PAGE analysis.

### Example 8: Downstream Process for AAV-ABCA4 Production

The downstream process for the AAV-ABCA4 vector is centred around the use of three monolith chromatography columns of different chemistries, which forms the basis an efficient and robust solution for AAV vector purification. Monoliths are especially suited to the purification of macromolecules, such as viral vectors, due to their large flow channels which allow ligand-target interactions to take place in a diffusion independent manner.

The first unit operation in the purification train is a hydrophobic interaction chromatography (HIC) capture step which is operated in a bind and elute mode. To facilitate binding of the vector to the column it is necessary to increase the concentration of the salting out agent by the dilution of the feed stream with a high molarity stock solution. Product elution is achieved using a step change to a lower molarity salt buffer.

Post the HIC step, the feed stream requires further conditioning to allow the vector to bind the negatively charged strong cation exchange (CEX SO3) column. The conditioning buffer protonates the AAV vector and reduces the counter ion concentration, thereby allowing the vector to bind to the negatively charged ligands. A filtration step is performed after the feed conditioning to remove any particulates and to preserve the effectiveness of the SO3 chromatography column. The SO3 step is also operated in a bind and elute mode with the elution taking place under a step increase in the salt concentration.

The enrichment, for full AAV particles, is achieved by exploiting the minor charge variation that exists between full and empty particles. A linear salt gradient elution utilising a strong anion exchange chromatography (AEX QA) column, allows an adequate resolution between the full and empty species. As with all the other unit operations, a conditioning of the feed is required to allow the vector to bind to the chromatography support.

Final vector concentration and dia-filtration is achieved using a 100 kDa, TFF mPES membrane. The product is diafiltered into the final formulation buffer (20 mM Tris pH 8.0, 1 mM MgCl2, 200 mM NaCl, 0.001% poloxamer 188). Prior to final formulation, in-process samples are analysed for vector recovery using a qPCR method to determine vector titre and yield of DNase Resistant Particles (DRP). This data is used to estimate the final volume required to achieve the final target titre. The excipient poloxamer 188 is added manually to process stream at a final concentration of 0.001% (v/v). After final formulation, the product is terminally sterile filtered through a 0.22 μm filter to yield the Purified Bulk Drug Substance (PBDS). Filling the PBDS completes the process and yields the Final Drug Product (FDP). Release testing takes place on both the PBDS and FDP.

Downstream Manufacturing Process Description

Hydrophobic Interation Chromatography Capture Step

The capture step of the clarified harvest material is performed using a hydrophobic interaction chromatography column. To ensure that the vector binds to the hydrophobic support, an increase in the molarity is required, and is achieved by the addition of a 2.6 M potassium phosphate, 2% sorbitol, pH 7 spike buffer. A 1:1 volumetric addition is performed by adding the dilution buffer to the clarified harvest, whilst the resulting solution is adequately agitated.

An OH monolith column (2 μm pore) is used as the chromatography unit for this unit step. A pulse test is to be performed on the column before use to ensure that the integrity of the column has not been compromised.

After sanitization and equilibration of the column, the product is loaded onto the monolith. Two washes are performed in order of decreasing molarity before product elution is achieved using an isocratic change to a lower molarity salt buffer (0.73 M potassium phosphate, 1% sorbitol, pH 7).

The eluate can be stored at 2-8° C. overnight, prior to forward processing (limit of storage duration to be determined).

FIG. 268 shows the process flow associated with the HIC chromatography unit operation. FIG. 269 shows parameters and associated operating ranges or setpoints which are to be used for the HIC capture step. FIG. 270 shows the steps required specifically for the chromatography procedure.

FIG. 271 shows a representative chromatogram which illustrates a typical full chromatograph HIC profile. FIG. 272 shows a representative chromatogram which provides more clarity by zooming in on the wash, elution and CIP stages.

FIG. 273 shows the details of the buffers that correspond to the stages listed in FIG. 270. FIG. 274 shows the details of the key materials and consumables that are to be utilized in the HIC chromatography step.

Cation Exchange Chromatography

A cation exchange based intermediate polishing chromatography step further reduces process impurities. The eluate from the HIC step needs to be conditioned to allow the vector to bind to the negatively charged ligands. The first part of the conditioning entails lowering the pH of the process stream which is required to be below the iso-electric point (pI), thereby giving the vector an overall positive surface charge. The dilution step also reduces the conductivity of the load, which reduces competitive binding from counter ions in solution. After adjustment of the process stream, a filtration step (0.8/0.45 μm combination filter) is performed to remove any particulates and preserve the effectiveness of the SO3 chromatography column. The neutralisation buffer is added to the added to restore the pH to near physiological levels. The eluate can be stored at 2-8° C. overnight, prior to forward processing (limit of storage duration to be determined). FIG. 275 outlines the steps required to perform the SO3 chromatography unit operation.

An SO3 monolith column (2 μm pore) is used as the chromatography unit for this unit step. A pulse test is to be performed on the column before use to ensure that the integrity of the column has not been compromised. FIG. 275 shows the flow chart of the SO3 chromatography unit operation. FIGS. 276 and 277 detail of the parameters and operating range/set points employed for the SO3 chromatography step.

FIG. 278 shows a representative typical full chromatogram. FIG. 279 shows a focus on the post load activities i.e. column washes, elution and the CIP step. FIG. 280 shows the details of the buffers used for this step. FIG. 281 shows the details of the exemplary materials and consumables used in the centrifugation concentration step.

QA Chromatography Step

The enrichment, for full AAV particles, is achieved by exploiting the minor charge variation that exists between full and empty particles. A linear gradient elution utilising an anion exchange chromatography column, allows an adequate resolution between the full and empty species, which permits peak cutting methods to be employed. Due to the minimal charge variation that exists, a step elution would not form the basis of a robust separation operation. A bioprocess system that can accurately and reproducibly form gradients, along with the ability to monitor UV absorbance signals is required to allow elution profile to be effectively formed and monitored. The neutralisation buffer is added to the added to restore the pH to near physiological levels. The eluate can be stored at 2-8° C. overnight, prior to forward processing (limit of storage duration to be determined).

A QA monolith column (2 μm pore) is used as the chromatography unit for this unit step. A pulse test is to be performed on the column before use to ensure that the integrity of the column has not been compromised. FIG. 282 shows the flow chart of the QA chromatography unit operation. FIG. 283 shows the parameters and associated operating ranges and setpoint which are to be used for the QA chromatography step. FIG. 284 shows the specific steps associated with the chromatography run. FIG. 285 and FIG. 286 show representative chromatograms (full and gradient elution respectively).

The elution collection criteria has been developed using the A260 and A280 wavelengths. The start of the collection is initiated at the crossing point of the A260 and A280 traces, which corresponds to the E3 fraction. Note: the A260 and A280 wavelengths need to be represented on the same scale for the criteria to be meaningful. The end of the peak collection takes place 3.5 CVs after the start of the collection. A collection criteria that achieves the same goal but uses a different method is acceptable; which will be the case where only one wavelength can be monitored. FIG. 287 shows QA buffer conditions and target specifications. FIG. 288 shows key materials/consumables used in the QA chromatography unit operation.

Tangential Flow Filtration and Excipient Addition

Final vector formulation is achieved using a 100 kDa, TFF mPES membrane. Prior to final formulation, in-process samples are analysed for vector recovery using a qPCR method to determine vector titre and yield of DNase Resistant Particles (DRP). This data is used to estimate the final volume required to achieve the final target titre. The product is diafiltered into the final formulation buffer (20 mM Tris pH 8.0, 1 mM MgCl2, 200 mM NaCl, 0.001% poloxamer 188). FIG. 289 shows graphical overview of the steps required to complete the tangential flow filtration step. FIG. 290 shows parameter and operating ranges for the tangential flow filtration step. FIG. 291 shows the details of the key materials and consumables that are to be used in the tangential flow filtration unit operation.

In-Process Hold Conditions

FIG. 292 shows the details of the hold times at in-process points that have been used during the process development of the AAV product.

### Example 9: ABCA4 Purification Process Optimization

Proxy Vector

A proxy vector was used for the compatability study. The proxy vector has the same capsid (AAV8/Y773F) as the ABCA4 vector. The capsid is the determining factor for the behavior of the vector across the HIC and SO3 steps. The packaged genome has an effect on the behavior over the QA step. The exemplary packaged genome used was wild type Bestrophin-1 due to its small size, however, this step employs a linear gradient and it is therefore anticipated that there would be no changes to the operating conditions for the QA step when using, for example, an ABCA4 transgene or other transgene of similar size.

Optimization of the HIC Capture Step

Optimization of the chromatography process for the ABCA4 vector has been performed. Changes were made to the wash buffer for the HIC process and the elution buffer for the CEX process. FIG. 323 details the HIC step parameters optimized by the use of a gradient elution run and shows an exemplary HIC chromatogram. The optimized peak cutting annotation was a 1.02M buffer. The non-optimized peak cutting annotation was a 1.08M buffer. The post load wash 2 buffer (W2) was adjusted from 1.08 M potassium phosphate, 1% sorbitol, pH 7.0 to 1.02M potassium phosphate, 1% sorbitol, pH 7.0. The reduction in molarity of the post load W2 buffer reduces the carryover of process related impurities into the elution fraction. All other operating parameters remained constant. In particular embodiments, the HIC Wash buffer used for RPGR vectors is 1.08 M K₂HPO4+KH₂PO4+1% sorbitol, pH 7.0, and the HIC Wash Buffer used for ABCA4 vectors is 1.02 M K₂HPO4+KH₂PO4+1% sorbitol, pH 7.0.

Optimization of the CEX Step

The CEX step was optimized by the use of a gradient elution run. FIG. 324 shows an exemplary chromatogram of the CEX run using the optimized elution buffer (E1). The optimized peak cutting annotation was a 1.33M buffer and the non-optimized peak cutting annotation was a 1.3M buffer. The E1 buffer was changed from 50 mM acetate, 1.3M NaCL, 0.1% poloxamer 188, pH 3.6 to 50 mM acetate, 1.33M NaCl, 0.1% poloxamer 188, pH 3.6. The increase in the molarity of E1 improves the step recovery of the CEX step. In particular embodiments, the CEX Elution Buffer used for RPGR is 0.05 M acetate+1.3 M NaCl+0.1% Poloxamer 188, pH 3.6±0.05, and the CEX Elution Buffer used for ABCA4 vectors is 0.05 M acetate+1.33 M NaCl+0.1% Poloxamer 188, pH 3.6±0.05.

FIG. 325 shows an exemplary optimized condition run through using both the optimized HIC and CEX chromatography steps. The exemplary QA chromatogram is run using the exemplar packed genome of wild type BEST-1 due to its small size. FIG. 326 details the step recovery for each elution. FIG. 327A details the Full:Empty vector results over the QA separation step by MALS. FIG. 327B details the Full:Empty vector results over the QA separation step by MALS and TEM.

### Example 10: Effect of Transfection Conditions on AAV Product Quality

The aim of this project was to identify transfection conditions that produce high quality AAV product. HEK293 cells were transfected with various ratios of plasmid DNA, (i.e., a plasmid encoding an AAV Construct comprising an RPGR^{ORF15} sequence (ITR), a plasmid encoding AAV8 rep and cap genes (RepCap), and a pHelper plasmid) using a polyethylenimine (PEI) transfection reagent, PEIpro® (Polyplus Transfection). The plasmid DNA/PEIpro® mixture was added to the cells, which were incubated at 37° C., 5% CO2 for 96 hours before being harvested, and the resulting AAV viral particles were evaluated. Four transfection conditions were evaluated, and the number of vector particles (Capsid ELISA) and the number of particles that contain the genome insert (Genomic titre) were quantified for each condition. FIG. 328A shows the transfection conditions tested, including the PEI:DNA (mL:mg) ratios and the plasmid molar ratios that were evaluated. FIG. 328B shows a graph quantifying the percentage of full particles, deduced from the ratio of the capsid ELISA and genomic titre results, which were calculated and highlight the differences between the conditions. FIGS. 328C and 328D show graphs of quantification values for the genomic titre (GC/mL) and capsid ELISA (particles/mL) resulting from transfection conditions 1, 2, 3, and 4, as shown in FIG. 323A, respectively.

Orthogonal Full to Empty Quantification

It is believed that the full particle analysis in FIG. 328(A-D) underestimates actual values, however, the trends are valid. Therefore, samples from these four conditions (FIG. 328B) were measured by an orthogonal method. FIG. 329A shows a representative graph of quantification of full particles to empty particles as measured using the orthogonal method. FIG. 329B shows a table of experimental conditions and results. The results mirrored the trend in FIG. 328(A-D). A comparison with an earlier result using material generated with a different transfection reagent (CaPO₄), suggests that the choice of transfection agent may also have an effect on the ratio of full:empty particles, and that using PEI results in a higher percentage of full particles, which may be enhanced by using molar ratios of the three plasmids wherein there is a higher relative amount of ITR as compared to Rep-Cap or pHelp plasmid and/or using a PEI:DNA (mL:mg) ratio of 4:1 or less, e.g., 2:1.

Effect of Transfection Agent (PEI vs. CaPO₄) on AAV Full to Empty Ratios

A PEI vs. CaPO₄ comparison transfection study was conducted to determine which reagent resulted in superior product. Material generated from PEI transfection or CaPO₄ was used to quantify full to empty vector ratios by HPLC. The material had not been through a process setep that would enrich for full particles. Previous variable conditions were kept constant between the two transfection conditions, including total DNA, PEI/DNA ratio and ratio of transfection plasmids. FIG. 330 shows a representative graph showing a comparison of full vector particle (%) analysis as a function of CaPO₄ vs. PEI transfection as measured by FLD (left bar for each reagent) or MALS (right bar for each reagent). The results from this side by side study further demonstrate that using PEI as a transfection agent, in lieu of CaPO₄, results in a higher ratio of full vector particles.

### Example 11: Downstream Process for AAV8-ABCA4 Production

The aim of the project was to develop an industrial chromatographic downstream process (DSP) for rAAV/Y733F ABCA4 late stage clinical and commercial program. The project included all developed steps—capture, intermediate polishing and separation of empty-full (E/F) AAV8 capsids using Macro-porous OH, SO3 and QA columns, and buffer exchange achieved by dialysis. Development was based on crude harvest material where PEI was used as a transfecting agent.

Materials and Methods

Sample

Sample was Berzonase™ treated and formulated in DMEM medium. The sample was an ABCA4 proxy vector having the same capsid (AAV8/Y733F) as the ABCA4 vector. The volume shipped was 4 L, the titer was 2.24E+10 vp/mL, and the total vector was 8.96+13 vector genomes (vg).

FPLC Systems (Preparative Runs)

### FPLC 1:

GE Healthcare Akta Explorer 100, UV flow cell 2 mm
0.75 mm I.D. capillaries (used with 8 mL and 1 mL column)
Sample loading: loading via system pump
Detection: UV 280 nm, UV 260 nm, conductivity, pH

HPLC Systems (Analytical Runs)

### HPLC 1:

PATfix™, 10 mL pump heads, 0.25 mm I.D. capillaries
Sample loading: 500 μL sample loop
Detection: UV 280 nm, UV 260 nm, fluorescence 280/348 (FLU, FLD), conductivity, MALS
Flow rate: 1-2 mL/min

Monolith Stationary Phases

Analytics runs (2 columns):
Macro-porous Adeno-0.1
Macro-porous SO3-0.1
   Preparative runs (3 columns):
Macro-porous OH-80
Macro-porous SO3-1
Macro-porous QA-1

Buffers

Buffers were prepared in fresh purified water and filtered through 0.22 μm filters. FIG. 336 shows buffers used for preparative and analytical runs.

Chromotographic Methods

Preparative Runs:
HIC Step—HIC purification step was performed using step gradients and with dedicated buffers as shown in FIG. 337.
CEX Step—CEX purification step was performed using step gradients and with dedicated buffers as shown in FIG. 338.
AEX Step—AEX purification step was performed using linear gradient from 0 to 100% mobile phase B in 60 column volumes (CVs) and then stepped to 100% MPC for 10 CVs, as shown in FIG. 339.

Analytic Runs:
Fingerprint—linear gradient from 0 to 35% mobile phase B in 50 CV, then from 35 to 100% in 5 CV; CIMac™ Adeno-0.1 column was used.
Total—linear gradient from 0 to 100% mobile phase B in 50 CV; CIMac™ SO3-0.1 column was used.
Empty/Full—Linear gradient from 0 to 40% mobile phase B in 50 column volumes (CV), then from 40 to 100% in 10 CV; CIMac™ Adeno-0.1 column was used.

SDS-PAGE

SDS-PAGE was carried out with a Mini-Protean II electrophoresis Cell (Bio-Rad) using 4-20% gradient gels under reducing conditions according to the manufacturer's instructions (Bio-Rad). The gels were run at 200 V for 35 min using a discontinuous Tris-glycine buffering system. Protein bands were visualized by Plus one Silver staining reagent (GE Healthcare). A 10-200 kDa molecular weight standard was used (PageRuler™ Unstained, thermos Fisher Scientific). Each time 20 ul of sample in appropriate dilution, was loaded to the well.

TEM

Samples were prepared for examination with TEM using negative staining method. Thawed samples were mixed gently and applied on freshly glow-discharged copper grids (400 mesh, formvar-carbon coated) for 5 minutes, washed and stained with 1 droplet of 1% (w/v) water solution of uranyl acetate.

The grids were observed with transmission electron microscope Philips CM 100 (FEI, The Netherlands), operating at 80 kV. At least 10 grid squares were examined thoroughly and several micrographs (camera ORIUS SC 200, Gatan, Inc.) were taken to evaluate the ratio between full and empty particles. Micrographs were taken coincidentally at different places on the grid.

ddPCR

Samples (and control) were DNAze treated and diluted in three points in duplicates (6 reactions for each sample). Reaction mix: ddPCR Supermix for Probes (no dUTP). Reaction volume: 20 uL, DNA volume 5 uL, Droplet volume 0.000739. Equipment used: Bio-Rad QX100™ Droplet Digital™ PCR System, Bio-Rad QX200™ AutoDG™ Droplet Digital™ PCR System, Fluidigm Biomark HD. Primers and probes used were determined based on the target detected.

Results and Discussion

Capture Step on Hydrophobic Interaction Chromatography (HIC) Using Macro-Porous OH Columns HPLC Analytical Methods

Preparative Run

Clarified harvest material (1.2 L divided in two bottles each containing 0.6 L) was thawed at room temperature, pooled and diluted 1:1 (1.2 L harvest+1.2 L buffer) with dilution buffer. Loading to the column using system pump at 5 CV/min. The run was the eighth (8) run for HIC conditions (HIC-8). FIG. 340 details the preparative run conditions. FIGS. 341A and B show a chromatogram from run HIC-8.

HPLC Total Analytics

Total particle method was used on HPLC for determination of chromatographic recovery. Fractions were desalted using Amicon Ultra 0.5. Main elution was further diluted 10× prior injection. FIGS. 342A-J show exemplary chromatograms based on HPLC analysis. From FIG. 342J, it is confirmed that all AAV bound to the column, and eluted in fractions W2, E1 and W3. When observing FIG. 342J (overlay) it was observed that both fractions W2 and W3 had other protein impurities present compared to main E1 elution. It must be accounted for that faction E1 is 10-fold diluted compared to other two, so loss of vector in fractions surrounding eluate was negligible. Areas of peaks were compared to load and harvest area peaks, to determine recoveries.

Recovery of Preparative Run

Recoveries for capture step HIC-OH comparing to starting clarified harvest material were 76% and 71% for ddPCR and HPLC Total analytics (MALS), respectively. The discrepancy between other methods in other detectors (A260, A280, FLD) was mainly caused by high salt concentration in sample, moreover the mass balances are not 100% in both cases, so normalization of two (ddPCR and HPLC Total analytics (MALS) would result in more accurate results with average 72%±2% recovery of AAV in main fraction. FIG. 343 details recoveries of HIC-8 run based on ddPCR and HPLC total analytics. FIG. 344 is a representative SDS-PAGE result for HIC-8 run. FIG. 344 portrays concentration of AAV and successful capture was achieved from clarified harvest material. Main elution after HIC step was highly concentrated but had many protein impurities that were removed by next chromatography step CEX-SO3.

Intermediate Polishing on Cation Exchange Chromatography (CEX) Using SO3 Column

Entire elution (E1) from HIC-OH was prepared to match binding conditions and loaded to CEX-SO3 column (SO3-7). FIG. 345 provide details on the parameters of the run. FIGS. 346A and B provide a chromatogram from run SO3-7. FIGS. 347A-J provide chromatograms based on HPLC analytics—Total method for SO3-7. From FIGS. 347A-J, it can be confirmed that all AAV bound to the column, and eluted in fractions E1 and W3. It must be accounted for that fraction E1 was 5-fold diluted compared to W3 so loss of vector in W3 was negligible. Areas of peaks were compared to load and initial HIC-8 R1 material to determine recoveries. FIG. 348 provides recoveries based on ddPCR and HPLC Total analytics for preparative run SO3-7. Recoveries for intermediate polishing step CEX-SO3 compared to starting HIC-8 E1 material were 90% and 86% for ddPCR and HPLC Total analytics (MALS), respectively. The discrepancy between the two methods was minor. In case of HPLC analytics, mass balance was not 100%. Normalization of two (ddPCR and HPLC Total analytics (MALS)) resulted in more accurate value with average 97% recovery of AAV in main fraction.

HPLC Total Analysis

Total particle method was used on HPLC for determination of chromatographic recovery. Fraction E1 was 5-fold diluted prior to injection.

SDS-PAGE

All fractions were loaded to the gel either neat or diluted under reducing conditions. FIG. 349 portrays further concentration of AAV, since 8-fold lower column size was used from HIC to CEX step. Main elution after HIC step has other protein impurities present apart from HIC to CEX step. In wash 3, there is a small portion of AAV band visible. The majority of host cell proteins are removed by strip with CIP.

Empty and Full AAV Capsids Separation on Anion Exchange Chromatography (AEX) Using CIM QA Column

Preparative Run

Entire elution (E1) from SO3-7 was diluted to match binding conditions and loaded to AEX-QA column. The run was the third (3) run for AEX conditions (QA-3). FIG. 350 details the preparative run conditions. FIGS. 351A and B show an exemplary chromatogram from run SO3-7.

HPLC Total Analytics

Empty-full method was used on HPLC for determination of chromatographic recovery and purity (ratio of E.F capsids). Fractions were diluted prior injection. FIGS. 352A-H show exemplary chromatograms based on HPLC analytics-Total method for SO3-7. From FIGS. 352A-H, we can confirm that all AAV binds to the column, since no peaks were visible in FT+W fraction. Due to slight difference in charge, empty capsid starts to elute first (E2) which are followed by full capsids found in E3. The difference in A260/A280 ratios confirms that AAV are pure in empty or full capsids. Values of 0.6 in A260/A280 ratios correspond to empty capsids, with predominantly protein composition, where full capsids which have DNA insert give a value of 1.3 and higher depending upon purity. Fraction E4 was collected separately since lower purity was obtained due to empty capsid contamination from the next eluting peak. E5 fraction was predominantly empty, aggregated and damaged capsids (two peaks), there is no AAV elution in E6 fraction. Areas of peaks were compared to load and initial SO3-7 E1 material, to determine recoveries and purity.

Dialysis

Buffer exchange was achieved by implementation of dialysis on QA-3 E3 sample. Details of the dialysis method are provided in FIG. 353. The end volume of sample was 3 mL.

Recovery of Preparative Run

Recoveries for the preparative run are summarized in FIGS. 354A-C. Recoveries for full capsid enrichment step (empty and full separation) step AEX-QA comparing to starting SO3-7 E1 material was 72% for ddPCR and 61% for HPLC Total analytics based on MALS detection (FIG. 354A). In both cases (ddPCR and HPLC analytics), mass balance was not reaching 100%. If percentage of main fraction was normalized to mass balance percentages for corresponding detector/assay, values of 83%±3% were obtained. Approximately 61% recovery was obtained after dialysis (not accounting for sample loss (0.66 mL)).

Based only on initial volume and end volume and their genomic value a DSP yield of 28% (after dialysis) or 45% (QA main fraction) was obtained, however it must be taken into account that sampling of main fractions after each purification step had a significant impact on overall recovery on smaller scale where end volumes are low. More accurate representation of DSP yield was achieved by accounting for losses after each purification step. By accounting normalized values (FIG. 354C), a total DSP yield of approximately 58% after chromatography steps and 42% after dialysis was reached.

Purity

FIG. 355 indicates that purity (percentage of full capsids) of main E3 fraction was approximately 100% if both MALS and FLD are taken in account. Since extinction coefficients for both absorbencies are not known, this makes MALS a more reliable detector, since it measure the diameter of the particle. Next in line was FLD regarding accuracy. The ratio changes in base of mean peak elution (fraction E4) where ratio was only 80-83%. The reason for collection of only 3.5% CV (approximately 80% peak) was achieving higher purity in E3 and only a minor loss of vector (E4) (11%—FIGS. 354A-C).

Purity was additionally tested by TEM, for ratio after SO3 intermediate step, QA-3 E3 (full capsids) and final sample after dialysis (FULL AAV). All grids expressed appropriate quality for observation and all three samples, SO3-7 E1, FULL AAV, and QA-3 E3 were clear, without impurities and without aggregation of particles. Sample SO3-7 E1 contained only 50% of full particles, while the percentage of full particles in other two samples was higher (79% in sample FULL AAV and 88% in sample QA-3 E3; FIG. 356). FIG. 357 shows: SO3-7 E1 (above; A and B), QA-3 E3 (middle; C and D) and after dialysis (below; E and F) evaluated by TEM. Left (A, C and E): low magnification, right (B, D and F): magnification used for counting.

SDS-PAGE

All fractions were loaded to the gel either neat or diluted under reducing conditions. FIG. 358 portrays that all fractions from E2 to E5 contain AAV. The protein band above 200 kDa mark present in E3 and E4 fractions corresponds to DNA insert found only in full capsids, indicating those two fractions contain full capsids, which complements HPLC E/F analytics results. Other protein impurities are found in E3 fraction aside VP1-VP3. Those impurities were not removed by dialysis (AAV8-PD), however, on a higher scale where TFE with MWCO 100 kDa was used, the additional bands were expected to be successfully removed.

HPLC Analysis—Fingerprint Method

FIGS. 359A and B show that the majority of impurities were removed by HIC step (FIG. 359A). The sample was further purified by polishing on CEX-SO3. The eluate from this stage was mainly pure and highly concentrated, but still consisted on both empty and full capsids. Last AEX-QA step separated the two capsids, and therefore isolated and enriched for full capsids. By comparing harvest material to QA main fraction, the AAV peak from starting material was identified.

### CONCLUSIONS

A downstream purification run was performed using clarified harvest as a starting material.

Capture and concentration of AAV was achieved by HIC-OH step, where proteins were found in flow through and AAV was bound to the column. Protein impurities were removed in either W2 or W3 fractions. The majority of protein impurities remaining in main elution fraction (E1) after HIC step were removed by the intermediate polishing step using CEX-SO3 column, where additional concentration of AAV was achieved by implementation of an 8-fold lower column scale. The percentage of full capsid at this state was approximately 50-65%, so full particle enrichment using AEX-QA was performed. After separating full capsids from empty capsids, a buffer exchange into formulation buffer was performed using dialysis. The entire downstream process yield from clarified harvest to completion of dialysis was 42% (after chromatography steps—58%) and purity of approximately 90% full AAV capsids was reached. The process was successfully performed at manufacturing scale.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A method of purifying a recombinant AAV (rAAV) particle from a mammalian host cell culture, comprising the steps of:
(a) purifying the plurality of rAAV particles through hydrophobic interaction chromatography (HIC) to produce a HIC eluate comprising the plurality of rAAV particles;
(b) purifying the HIC eluate of (a) through cation exchange chromatography (CEX) to produce a CEX eluate comprising a plurality of rAAV particles;
(c) isolating a plurality of full rAAV particles from the CEX eluate of (b) by anion exchange (AEX) chromatography to produce a AEX eluate comprising a purified and enriched plurality of full rAAV particles; and
(d) diafiltering and concentrating the AEX eluate from (c) into a formulation buffer by tangential flow filtration (TFF) to produce a final composition comprising a purified and enriched plurality of full rAAV particles and the final formulation buffer.

2. The method of claim 1, wherein the method further comprises the steps of
contacting a plurality of transfected mammalian host cells and a virus release solution under conditions suitable for the release of the plurality of rAAV particles into a harvest media to produce a composition comprising a plurality of rAAV particles, virus release solution and harvest media; and
purifying the plurality of rAAV particles from the composition through hydrophobic interaction chromatography (HIC) to produce a HIC eluate comprising the plurality of rAAV particles.

3. The method of claim 2, wherein the method further comprises the step of
culturing a plurality of mammalian host cells in a harvest media under conditions suitable for the formation of a plurality of rAAV particles, wherein the plurality of mammalian host cells have been transfected with a plasmid vector comprising an exogenous sequence, a helper plasmid vector, and a plasmid vector comprising a sequence encoding a viral Rep protein and a viral Cap protein to produce a plurality of transfected mammalian host cells, prior to the contacting step.

4. The method of claim 3, wherein the harvest media comprises one or more of Dulbecco's Modified Eagle's medium (DMEM), stabilized glutamine, stabilized glutamine dipeptide and Benzonase.

5. The method of claim 3, wherein the harvest media comprises glycine, L-Arginine hydrochloride, L-Cystine dihydrochloride, L-Glutamine, L-Histidine hydrochloride-H2O, L-Isoleucine, L-Leucine, L-Lysine hydrochloride, L-Methionine, L-Phenylalanine, L-Serine, L-Threonine, L-Tryptophan, L-Tyrosine disodium salt dehydrate, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl2) (anhyd.), Ferric Nitrate (Fe(NO3)3″9H2O), Magnesium Sulfate (MgSO4) (anhyd.), Potassium Chloride (KCl), Sodium Bicarbonate (NaHCO3), Sodium Chloride (NaCl), Sodium Phosphate monobasic (NaH2PO4-H2O), and D-Glucose (Dextrose).

6. The method of claim 4, wherein the harvest media comprises 4 mM stabilized glutamine or stabilized glutamine dipeptide.

7. The method of any one of claims 3-6, wherein the harvest media comprises a serum-free media.

8. The method of any one of claims 3-6, wherein the harvest media consists of a serum-free media.

9. The method of any one of claims 3-8, wherein the harvest media comprises a protein-free media.

10. The method of any one of claims 3-8, wherein the harvest media consists of a protein-free media.

11. The method of any one of claims 3-10, wherein the harvest media comprises a clarified media.

12. The method of any one of claims 3-10, wherein the harvest media consists of a clarified media.

13. The method of any one of claims 1-12, wherein the exogenous sequence comprises:
(a) a sequence encoding a rhodopsin kinase promoter;
(b) a sequence encoding a retinitis pigmentosa GTPase regulator ORF15 isoform (RPGR^{ORF15}); and
(c) a sequence encoding a polyadenylation (polyA) signal.

14. The method of claim 13, wherein the rhodopsin kinase promoter is a GRK1 promoter.

15. The method of claim 14, wherein the sequence encoding the GRK1 promoter comprises or consists of:
```
(SEQ ID NO: 5)
    1 gggccccaga agcctggtgg ttgtttgtcc ttctcagggg aaaagtgagg cggccccttg
  61 gaggaagggg ccgggcagaa tgatctaatc ggattccaag cagctcaggg gattgtcttt
121 ttctagcacc ttcttgccac tcctaagcgt cctccgtgac cccggctggg atttagcctg
181 gtgctgtgtc agccccggg.
```

16. The method of any one of claims 13-15, wherein the sequence encoding the RPGR^{ORF15} is a codon optimized human RPGR^{ORF15} sequence.

17. The method of claim 16, wherein the sequence encoding RPGR^{ORF15} comprises a nucleotide sequence encoding an amino acid sequence of:
```
(SEQ ID NO: 78)
      1 MREPEELMPD SGAVFTFGKS KFAENNPGKF WFKNDVPVHL SCGDEHSAVV TGNNKLYMFG
    61 SNNWGQLGLG SKSAISKPTC VKALKPEKVK LAACGRNHTL VSTEGGNVYA TGGNNEGQLG
  121 LGDTEERNTF HVISFFTSEH KIKQLSAGSN TSAALTEDGR LFMWGDNSEG QIGLKNVSNV
  181 CVPQQVTIGK PVSWISCGYY HSAFVTTDGE LYVFGEPENG KLGLPNQLLG NHRTPQLVSE
  241 IPEKVIQVAC GGEHTVVLTE NAVYTFGLGQ FGQLGLGTFL FETSEPKVIE NIRDQTISYI
  301 SCGENHTALI TDIGLMYTFG DGRHGKLGLG LENFTNHFIP TLCSNFLRFI VKLVACGGCH
  361 MVVFAAPHRG VAKEIEFDEI NDTCLSVATF LPYSSLTSGN VLQRTLSARM RRRERERSPD
  421 SFSMRRTLPP IEGTLGLSAC FLPNSVFPRC SERNLQESVL SEQDLMQPEE PDYLLDEMTK
  481 EAEIDNSSTV ESLGETTDIL NMTHIMSLNS NEKSLKLSPV QKQKKQQTIG ELTQDTALTE
  541 NDDSDEYEEM SEMKEGKACK QHVSQGIFMT QPATTIEAFS DEEVEIPEEK EGAEDSKGNG
  601 IEEQEVEANE ENVKVHGGRK EKTEILSDDL TDKAEVSEGK AKSVGEAEDG PEGRGDGTCE
  661 EGSSGAEHWQ DEEREKGEKD KGRGEMERPG EGEKELAEKE EWKKRDGEEQ EQKEREQGHQ
  721 KERNQEMEEG GEEEHGEGEE EEGDREEEEE KEGEGKEEGE GEEVEGEREK EEGERKKEER
  781 AGKEEKGEEE GDQGEGEEEE TEGRGEEKEE GGEVEGGEVE EGKGEREEEE EEGEGEEEEG
  841 EGEEEEGEGE EEEGEGKGEE EGEEGEGEEE GEEGEGEGEE EEGEGEGEEE GEGEGEEEEG
  901 EGEGEEEGEG EGEEEEGEGK GEEEGEEGEG EGEEEEGEGE GEDGEGEGEE EEGEWEGEEE
  961 EGEGEGEEEG EGEGEEGEGE GEEEEGEGEG EEEEGEEEGE EEGEGEEEGE GEGEEEEEGE
1021 VEGEVEGEEG EGEGEEEEGE EEGEEREKEG EGEENRRNRE EEEEEEGKYQ ETGEEENERQ
1081 DGEEYKKVSK IKGSVKYGKH KTYQKKSVTN TQGNGKEQRS KMPVQSKRLL KNGPSGSKKF
1141 WNNVLPHYLE LK.
```

18. The method of claim 16 or 17, wherein the sequence encoding RPGR^{ORF15} comprises or consists of a nucleotide sequence of:
```
(SEQ ID NO: 80)
      1 atgagagagc cagaggagct gatgccagac agtggagcag tgtttacatt cggaaaatct
    61 aagttcgctg aaaataaccc aggaaagttc tggtttaaaa acgacgtgcc cgtccacctg
  121 tcttgtggcg atgagcatag tgccgtggtc actgggaaca ataagctgta catgttcggg
  181 tccaacaact ggggacagct ggggctggga tccaaatctg ctatctctaa gccaacctgc
  241 gtgaaggcac tgaaacccga gaaggtcaaa ctggccgctt gtggcagaaa ccacactctg
  301 gtgagcaccg agggcgggaa tgtctatgcc accggaggca acaatgaggg acagctggga
  361 ctgggggaca ctgaggaaag gaataccttt cacgtgatct ccttctttac atctgagcat
  421 aagatcaagc agctgagcgc tggctccaac acatctgcag ccctgactga ggacgggcgc
  481 ctgttcatgt ggggagataa ttcagagggc cagattgggc tgaaaaacgt gagcaatgtg
  541 tgcgtccctc agcaggtgac catcggaaag ccagtcagtt ggatttcatg tggctactat
  601 catagcgcct tcgtgaccac agatggcgag ctgtacgtct ttggggagcc cgaaaacgga
  661 aaactgggcc tgcctaacca gctgctgggc aatcaccgga caccccagct ggtgtccgag
  721 atccctgaaa aagtgatcca ggtcgcctgc gggggagagc atacagtggt cctgactgag
  781 aatgctgtgt ataccttcgg actgggccag tttggccagc tggggctggg aaccttcctg
  841 tttgagacat ccgaaccaaa agtgatcgag aacattcgcg accagactat cagctacatt
  901 tcctgcggag agaatcacac cgcactgatc acagacattg gcctgatgta tacctttggc
  961 gatggacgac acgggaagct gggactggga ctggagaact tcactaatca ttttatcccc
1021 accctgtgtt ctaacttcct gcggttcatc gtgaaactgg tcgcttgcgg cgggtgtcac
1081 atggtggtct tcgctgcacc tcataggggc gtggctaagg agatcgaatt tgacgagatt
1141 aacgatacat gcctgagcgt ggcaactttc ctgccataca gctccctgac ttctggcaat
1201 gtgctgcaga gaaccctgag tgcaaggatg cggagaaggg agagggaacg ctctcctgac
1261 agtttctcaa tgcgacgaac cctgccacct atcgagggaa cactgggact gagtgcctgc
1321 ttcctgccta actcagtgtt tccacgatgt agcgagcgga atctgcagga gtctgtcctg
1381 agtgagcagg atctgatgca gccagaggaa cccgactacc tgctggatga gatgaccaag
1441 gaggccgaaa tcgacaactc tagtacagtg gagtccctgg gcgagactac cgatatcctg
1501 aatatgacac acattatgtc actgaacagc aatgagaaga gtctgaaact gtcaccagtg
1561 cagaagcaga agaaacagca gactattggc gagctgactc aggacaccgc cctgacagag
1621 aacgacgata gcgatgagta tgaggaaatg tccgagatga aggaaggcaa agcttgtaag
1681 cagcatgtca gtcaggggat cttcatgaca cagccagcca caactattga ggctttttca
1741 gacgaggaag tggagatccc cgaggaaaaa gagggcgcag aagattccaa ggggaatgga
1801 attgaggaac aggaggtgga agccaacgag gaaaatgtga aagtccacgg aggcaggaag
1861 gagaaaacag aaatcctgtc tgacgatctg actgacaagg ccgaggtgtc cgaaggcaag
1921 gcaaaatctg tcggagaggc agaagacgga ccagagggac gaggggatgg aacctgcgag
1981 gaaggctcaa gcggggctga gcattggcag gacgaggaac gagagaaggg cgaaaaggat
2041 aaaggccgcg gggagatgga acgacctgga gagggcgaaa aagagctggc agagaaggag
2101 gaatggaaga aaagggacgg cgaggaacag gagcagaaag aaagggagca gggccaccag
2161 aaggagcgca accaggagat ggaagagggc ggcgaggaag agcatggcga gggagaagag
2221 gaagagggcg atagagaaga ggaagaggaa aaagaaggcg aagggaagga ggaaggagag
2281 ggcgaggaag tggaaggcga gagggaaaag gaggaaggag aacggaagaa agaggaaaga
2341 gccggcaaag aggaaaaggg cgaggaagag ggcgatcagg gcgaaggcga ggaggaagag
2401 accgagggcc gcggggaaga gaaagaggag ggaggagagg tggagggcgg agaggtcgaa
2461 gagggaaagg gcgagcgcga agaggaagag gaagagggcg agggcgagga agaagagggc
2521 gagggggaag aagaggaggg agagggcgaa gaggaagagg gggagggaaa gggcgaagag
2581 gaaggagagg aaggggaggg agaggaagag ggggaggagg gcgaggggga aggcgaggag
2641 gaagaaggag agggggaagg cgaagaggaa ggcgaggggg aaggagagga ggaagaaggg
2701 gaaggcgaag gcgaagagga gggagaagga gagggggagg aagaggaagg agaagggaag
2761 ggcgaggagg aaggcgaaga gggagagggg gaaggcgagg aagaggaagg cgagggcgaa
2821 ggagaggacg gcgagggcga gggagaagag gaggaagggg aatgggaagg cgaagaagag
2881 gaaggcgaag gcgaaggcga agaagagggc gaaggggagg gcgaggaggg cgaaggcgaa
2941 ggggaggaag aggaaggcga aggagaaggc gaggaagaag agggagagga ggaaggcgag
3001 gaggaaggag agggggagga ggagggagaa ggcgagggcg aagaagaaga agagggagaa
3061 gtggagggcg aagtcgaggg ggaggaggga gaaggggaag gggaggaaga agagggcgaa
3121 gaagaaggcg aggaaagaga aaaagaggga gaaggcgagg aaaaccggag aaatagggaa
3181 gaggaggaag aggaagaggg aaagtaccag gagacaggcg aagaggaaaa cgagcggcag
3241 gatggcgagg aatataagaa agtgagcaag atcaaaggat ccgtcaagta cggcaagcac
3301 aaaacctatc agaagaaaag cgtgaccaac acacagggga atggaaaaga gcagaggagt
3361 aagatgcctg tgcagtcaaa acggctgctg aagaatggcc catctggaag taaaaaattc
3421 tggaacaatg tgctgcccca ctatctggaa ctgaaataa.
```

19. The method of any one of claims 13-18, wherein the sequence encoding the polyA signal comprises a bovine growth hormone (BGH) polyA sequence.

20. The method of claim 19, wherein the sequence encoding the BGH polyA signal comprises a nucleotide sequence of:
```
(SEQ ID NO: 83)
    1 cgctgatca gcctcgactg tgccttctag ttgccagcca tctgttgttt gcccctcccc
  61 cgtgccttcc ttgaccctgg aaggtgccac tcccactgtc ctttcctaat aaaatgagga
121 aattgcatcg cattgtctga gtaggtgtca ttctattctg gggggtgggg tggggcagga
181 cagcaagggg gaggattggg aagacaatag caggcatgct ggggatgcgg tgggctctat
241 ggcttctgag gcggaaagaa ccagctgggg.
```

21. The method of any one of claims 1-12, wherein the exogenous sequence comprises a sequence encoding an ATP Binding Cassette, Subfamily Member 4 (ABCA4) protein or a portion thereof.

22. The method of claim 21, wherein the exogenous sequence comprises a 5′ sequence encoding an ABCA4 protein or a portion thereof.

23. The method of claim 21, wherein the exogenous sequence comprises a 3′ sequence encoding an ABCA4 protein or a portion thereof.

24. The method of claim 21, wherein the exogenous sequence further comprises a sequence encoding a promoter.

25. The method of claim 24, wherein the exogenous sequence comprises a sequence encoding a rhodopsin kinase (RK) promoter

26. The method of claim 25, wherein the RK promoter is a GRK1 promoter.

27. The method of claim 26, wherein the sequence encoding the GRK1 promoter comprises or consists of:
```
(SEQ ID NO: 5)
    1 gggccccaga agcctggtgg ttgtttgtcc ttctcagggg aaaagtgagg cggccccttg
  61 gaggaagggg ccgggcagaa tgatctaatc ggattccaag cagctcaggg gattgtcttt
121 ttctagcacc ttcttgccac tcctaagcgt cctccgtgac cccggctggg atttagcctg
181 gtgctgtgtc agccccggg.
```

28. The method of claim 24, wherein the exogenous sequence further comprises a sequence encoding a chicken beta-actin (CBA) promoter.

29. The method of claim 28, wherein the sequence encoding the CBA promoter comprises or consists of:

| (SEQ ID NO: 16) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |
| or | |
| (SEQ ID NO: 24) | |
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

30. The method of any one of claims 21-29, wherein the sequence encoding the ABCA4 is a human ABCA4 sequence.

31. The method of claim 30, wherein the sequence encoding ABCA4 comprises a 5′ nucleotide sequence comprising nucleotides 1-3701 or 1-4326 of SEQ ID NO: 2 or SEQ ID NO: 1.

32. The method of claim 30, wherein the sequence encoding ABCA4 comprises a 3′ nucleotide sequence comprising nucleotides 3154-6822, 3196-6822, 3494-6822, 3603-6822, 3653-6822, 3678-6822, 3702-6822 or 3494-6822 of SEQ ID NO: 2 or SEQ ID NO: 1.

33. The method of any one of claims 1-32, wherein the plasmid vector comprising an exogenous sequence further comprises a sequence encoding a 5′ inverted terminal repeat (ITR) and a sequence encoding a 3′ ITR.

34. The method of any one of claims 1-33, wherein the sequence encoding the 5′ ITR and the sequence encoding the 3′ ITR are derived from a 5′ITR sequence and a 3′ ITR sequence of an AAV of serotype 2 (AAV2).

35. The method of any one of claims 1-34, wherein the sequence encoding the 5′ ITR and the sequence encoding the 3′ ITR comprise sequences that are identical to a sequence of a 5′ITR and a sequence of a 3′ ITR of an AAV2.

36. The method of any one of claims 1-34, wherein the sequence encoding the 5′ ITR comprises or consists of the nucleotide sequence of:
(SEQ ID NO: 34)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT.

37. The method of any one of claim 1-34 or 36, wherein the sequence encoding the 3′ ITR comprises or consists of the nucleotide sequence of:
(SEQ ID NO: 35)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

38. The method of any one of claims 1-37, wherein the exogenous sequence further comprises a sequence encoding a Kozak sequence.

39. The method of claim 38, wherein the Kozak sequence comprises the nucleotide sequence of GGCCACCATG (SEQ ID NO: 73).

40. The method of any one of claims 1-39, wherein the plasmid vector comprising an exogenous sequence, the helper plasmid vector or the plasmid vector comprising the sequence encoding a viral Rep protein and a viral Cap protein further comprises a sequence encoding a selection marker.

41. The method of any one of claims 1-40, wherein the sequence encoding the viral Rep protein and the sequence encoding the viral Cap protein comprise sequences isolated or derived from AAV serotype 8 (AAV8) viral Rep protein and viral Cap protein sequences.

42. The method of any one of claims 2-41, wherein the mammalian host cells have been transfected with a composition comprising one or more of a polymer, calcium phosphate, a lipid, and a vector capable of traversing a cell membrane.

43. The method of claim 42, wherein the polymer comprises polyethylenimine (PEI).

44. The method of claim 43, wherein the vector capable of traversing a cell membrane comprises a liposome, a micelle, or a nanoparticle

45. The method of claim 43, wherein the nanoparticle comprises carbon, silicon, or gold.

46. The method of claim 45, wherein the nanoparticle comprises a polymer.

47. The method of any one of claims 2-46, wherein the virus release solution comprises a salt and a high pH.

48. The method of claim 47, wherein the salt comprises NaCl.

49. The method of claim 47 or 48, wherein the high pH comprises a pH greater than or equal to 7.1.

50. The method of claim 41 or 42, wherein the high pH comprises a pH greater than or equal to 9.0.

51. The method of any one of claims 2-50, wherein conditions suitable for the formation of a plurality of rAAV particles comprise incubating the mammalian host cells for 18 hours at 37° C. and 5% CO2.

52. The method of any one of claims 2-50, wherein the conditions suitable for the formation of a plurality of rAAV particles comprises incubating the mammalian host cells at a CO2 level equal to or less than 10% CO2.

53. The method of any one of claims 1-52, wherein HIC step of (a) further comprises the steps of:
(i) generating a HIC chromatogram; and
(ii) selecting a fraction on the HIC chromatogram containing rAAV particles to produce the HIC eluate comprising a plurality of rAAV viral particles.

54. The method of claim 53, further comprising diluting the harvest media into a high salt buffer prior to generating the HIC chromatogram

55. The method of claim 53 or 54, wherein the plurality of rAAV particles are eluted using a step gradient.

56. The method of claim 55, wherein the step gradient comprises a decrease in salt concentration at each step gradient.

57. The method of any one of claims 1-56, wherein the CEX step of (b) further comprises the steps of:
(i) generating a CEX chromatogram; and
(ii) selecting a fraction from the CEX chromatogram containing rAAV particles to produce the CEX eluate comprising a plurality of rAAV viral particles.

58. The method of claim 57, wherein the CEX chromatography comprises an SO₃− cation exchange matrix.

59. The method of claim 57 or 58, further comprising adjusting the HIC eluate into a low salt buffer prior to generating the CEX chromatogram.

60. The method of claim 59, wherein the adjustment comprises a dilution step.

61. The method of claim 59, wherein the adjustment step comprises a TFF step.

62. The method of claim 61, wherein the TFF step is performed using a 100 kDa hollow fiber filter (HFF).

63. The method of claim 61, wherein the TFF step is performed using a 70 kDa HFF.

64. The method of claim 61, wherein the TFF step is performed using a 50 kDa HFF.

65. The method of claim 61, wherein the TFF step is performed using at least a 50, 55, 60, 65, 70, 75, 80, 85, 90, 95, or 100 kDa HFF or any number of kDa in between.

66. The method of any one of claims 57-62, wherein the pH of the HIC eluate is adjusted to pH 3.0 to pH 4.0, inclusive of the endpoints.

67. The method any one of claims 57-62, wherein the pH of the HIC eluate is adjusted to pH 3.5 to pH 3.7, inclusive of the endpoints.

68. The method of claim of any one of claims 57-67, further comprising filtering the HIC eluate.

69. The method of claim 68, wherein filtering the HIC eluate comprises a 0.8/0.45 μm polyethersulfone (PES) filter.

70. The method of any one of claims 57-69, wherein the plurality of rAAV particles are eluted using a step gradient.

71. The method of claim 70, wherein the step gradient comprises a pH gradient, a salt gradient or a combination thereof.

72. The method of any one of claims 57-69, wherein the plurality of rAAV particles are eluted using a linear gradient.

73. The method of claim 72, wherein the linear gradient comprises a pH gradient, a salt gradient or a combination thereof.

74. The method of any one of claims 57-73, further comprising neutralizing the pH of the CEX eluate.

75. The method of claim 74, wherein the pH of the neutralized CEX eluate is pH 9.0.

76. The method of any one of claims 1-75, wherein the AEX Chromatography step of (c) further comprises the steps of:
(i) generating an AEX chromatogram; and
(ii) selecting a fraction from the AEX chromatogram containing full rAAV particles to produce the AEX eluate comprising a purified and enriched plurality of full rAAV particles.

77. The method of claim 76, wherein the AEX chromatography comprises an Anion Exchange (QA) matrix.

78. The method of claim 76 or 77, further comprising adjusting the CEX eluate into a low salt buffer prior to generating the AEX chromatogram.

79. The method of claim 78, wherein the adjustment comprises a dilution step.

80. The method of claim 78, wherein the adjustment step comprises a TFF step.

81. The method of claim 80 wherein the adjustment step comprises a first TFF step and a second TFF step.

82. The method of claim 80, wherein the TFF step is performed using a 100 kDa hollow fiber filter (HFF).

83. The method of claim 81, wherein both the first and second TFF step is performed using a 100 kDa hollow fiber filter (HFF).

84. The method of any one of claims 78-83, wherein the diluted CEX eluate is pH 9.0.

85. The method of any one of claims 76-84, wherein the purified and enriched plurality of full rAAV particles are eluted using a linear gradient.

86. The method of any one of claims 76-84, wherein the purified and enriched plurality of full rAAV particles are eluted using a step gradient.

87. The method of any one of claims 76-86, further comprising neutralizing the pH of the eluate comprising the purified and enriched plurality of full rAAV particles.

88. The method of any one of claims 1-87, wherein the TFF step of (d) is performed using a 100 kDa hollow fiber filter (HFF).

89. The method of claim 88, wherein the method further comprises a second TFF, and wherein both the first and second TFF steps are performed using a 100 kDa HFF.

90. The method of any one of claims 1-89, wherein the final formulation buffer comprises Tris, MgCl₂, and NaCl.

91. The method of claim 90, wherein the final formulation buffer comprises 20 mM Tris, 1 mM MgCl₂, and 200 mM NaCl at pH 8.

92. The method of claim 90 or 91, wherein the final formulation buffer further comprises poloxamer 188 at 0.001%.

93. The method of any one of claims 1-92, further comprising adding pluronic F-68 to the final composition.

94. The method of claim 93, wherein the final composition comprising the purified and enriched plurality of full rAAV particles and the final formulation buffer is frozen at −80° C.

95. A composition comprising a plurality of rAAV particles produced by the method of any one of claims 1-94.

96. The composition of claim 95, wherein the composition comprises
(a) between 0.5×10¹¹ vg/mL and 1×10¹³ vg/mL, inclusive of the endpoints; and
(b) less than 30% empty capsids.

97. The composition of claim 95, wherein the composition comprises
(a) between 0.5×10¹¹ vg/mL and 1×10¹³ vg/mL, inclusive of the endpoints; and
(b) less than 25% empty capsids.

98. The composition of claim 96 or 97, wherein the composition comprises about 0.5×10¹¹ vg/mL.

99. The composition of claim 96 or 97, wherein the composition comprises about 1.0×10¹³ vg/mL.

100. The composition of claim 96 or 97, wherein the composition comprises about 5×10¹² vg/mL.

101. The composition of any one of claims 96-100, wherein a portion of the plurality of rAAV comprises a functional vector genome, wherein each functional vector genome is capable of expressing an exogenous sequence in a cell following transduction.

102. The composition of claim 101, wherein the portion of the plurality of rAAV comprising a functional vector genome expresses the exogenous sequence at a 2-fold increase when compared to a level of expression of a corresponding endogenous sequence in a nontransduced cell.

103. The composition of claim 101, wherein the portion of the plurality of rAAV comprising a functional vector genome expresses the exogenous sequence at a 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or any other increment fold increase in between, when compared to a level of expression of a corresponding endogenous sequence in a nontransduced cell.

104. The composition of any one of claims 101-103, wherein the exogenous sequence and the corresponding endogenous sequence are not identical.

105. The composition of claim 102 or 103, wherein the exogenous sequence and the corresponding endogenous sequence are not identical, but a protein encoded by the exogenous sequence and a protein encoded by the endogenous sequence are identical.

106. The composition of claim 104 or 105, wherein the exogenous sequence and the corresponding endogenous sequence have at least 70%, 75%, 80%, 85%, 90%, 95%, 97%, 99% or any percentage in between of identity.

107. The composition of any one of claims 95-106, wherein the exogenous sequence is codon-optimized when compared to the endogenous sequence.

108. The composition of 107, wherein the exogenous sequence and the corresponding endogenous sequence have at least 50%, 55%, 60%, 65%, 70%, 75%, 80%, 85%, 90%, 95%, 97%, 99% or any percentage in between of identity.

109. The composition of any one of claims 95-108, wherein following transduction of a cell with the composition, the exogenous sequence encodes a protein.

110. The composition of claim 109, wherein the protein encoded by the exogenous sequence has an activity level equal to or greater than an activity level of a protein encoded by a corresponding sequence of a nontransduced cell.

111. The composition of claim 110, wherein the exogenous sequence and the corresponding endogenous sequence are identical.

112. The composition of claim 110, wherein the exogenous sequence and the corresponding endogenous sequence are not identical.

113. The composition of claim 112, wherein the exogenous sequence and the corresponding endogenous sequence have at least 70%, 75%, 80%, 85%, 90%, 95%, 97%, 99% or any percentage in between of identity.

114. The composition of any one of claims 95-113, wherein following transduction of a cell with the composition, the exogenous sequence encodes a protein.

115. The method of any one of claims 3-114, wherein the plasmid vector comprising an exogenous sequence, the helper plasmid vector, and the plasmid vector comprising a sequence encoding a viral Rep protein and a viral Cap protein are at a molar ratio of about 0.5:1:1 to about 10:1:1 or about 1:1:1 to about 10:1:1, respectively, optionally about 1:1:1, about 2:1:1, about 3:1:1, about 4:1:1, about 5:1:1, about 6:1:1, about 7:1:1, about 8:1:1, about 9:1:1, or about 10:1:1, respectively, optionally wherein the cells were transfected using PEI.

116. The method of any one of claims 3-114, wherein the plasmid vector comprising an exogenous sequence, the helper plasmid vector, and the plasmid vector comprising a sequence encoding a viral Rep protein and a viral Cap protein are provided in a molar ratio of about 3:1:1, respectively, optionally wherein the cells were transfected using PEI.

117. The method of any one of claims 3-114, wherein the plasmid vector comprising an exogenous sequence, the helper plasmid vector, and the plasmid vector comprising a sequence encoding a viral Rep protein and a viral Cap protein are provided in a molar ratio of about 10:1:1, respectively, optionally wherein the cells were transfected using PEI.

118. The method of any one of claims 3-114, wherein the molar ratio of the plasmid vector comprising an exogenous sequence (pITR) to the helper plasmid vector (pHELP) is between 1:1 and 20:19, optionally wherein the cells were transfected using PEI.

119. The method of any one of claims 3-114, wherein the molar ratio of the pITR to the plasmid vector comprising a sequence encoding a viral Rep protein and a viral Cap protein (pREPCAP) is between 1:1 and 20:19, optionally wherein the cells were transfected using PEI.

120. The method of any one of claims 115-119, wherein the culturing a plurality of mammalian host cells in a harvest media under conditions suitable for the formation of a plurality of rAAV particles comprises culturing in the presence of a transfection agent.

121. The method of claim 120, wherein the transfection agent comprises calcium phosphate (CaPO₄).

122. The method of claim 120, wherein the transfection agent comprises polyethylenimine (PEI).

123. The method of claim 122, wherein the transfection agent comprises PEI and DNA at a ratio of about 5:1 to about 1:1 (mL:mg), respectively, optionally about 2:1 to about 4:1, about 4:1, about 3:1, or about 2:1.

124. The method of claim 122 or 123, wherein the transfection agent comprises PEI and DNA, wherein the DNA comprises a plasmid vector comprising an exogenous sequence, a plasmid vector comprising a sequence encoding a viral Rep protein and a viral Cap protein, and a helper plasmid at a molar ratio of about 0.5:1:1 to about 10:1:1 or about 1:1:1 to about 10:1:1, respectively, optionally about 2:1:1, about 3:1:1, about 4:1:1, about 5:1:1, about 6:1:1, about 7:1:1, about 8:1:1, about 9:1:1, or about 10:1:1.

125. A method of producing a recombinant AAV vector, comprising transfecting mammalian host cells with:
(i) a plasmid vector comprising an exogenous sequence;
(ii) a plasmid vector comprising a sequence encoding a viral Rep protein and a viral Cap protein; and
(iii) a helper plasmid vector,
wherein the mammalian host cells are contacted with a transfection medium comprising the plasmid vector comprising the exogenous sequence, the plasmid vector comprising a sequence encoding a viral Rep protein and a viral Cap protein, and the helper plasmid at a molar ratio of about 1:1:1 to about 10:1:1, respectively, optionally about 2:1:1, about 3:1:1, about 4:1:1, about 5:1:1, about 6:1:1, about 7:1:1, about 8:1:1, about 9:1:1, or about 10:1:1.

126. The method of claim 125, wherein the transfection medium comprises a transfection agent selected from polyethylenimine (PEI) and CaPO₄.

127. The method of claim 126, wherein the transfection agent is PEI, and wherein the tranfection medium comprises PEI and DNA at a ratio of about 5:1 to about 1:1, about 2:1 to about 4:1, about 4:1, about 3:1, about 2:1, or about 1:1.

128. The method of any one of claims 125-127, wherein the exogenous sequence comprises:
(a) a sequence encoding a rhodopsin kinase promoter;
(b) a sequence encoding a retinitis pigmentosa GTPase regulator ORF15 isoform (RPGR^{ORF15}); and
(c) a sequence encoding a polyadenylation (polyA) signal.

129. The method of claim 128, wherein the rhodopsin kinase promoter is a GRK1 promoter.

130. The method of claim 129, wherein the sequence encoding the GRK1 promoter comprises or consists of:

| (SEQ ID NO: 5) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

131. The method of any one of claims 128-130, wherein the sequence encoding the RPGR^{ORF15} is a codon optimized human RPGR^{ORF15} sequence.

132. The method of claim 131, wherein the sequence encoding RPGR^{ORF15} comprises a nucleotide sequence encoding an amino acid sequence of:

| (SEQ ID NO: 78) | |
|---|---|
| 1 | MREPEELMPD SGAVFTFGKS KFAENNPGKF WFKNDVPVHL SCGDEHSAVV TGNNKLYMFG |
| 61 | SNNWGQLGLG SKSAISKPTC VKALKPEKVK LAACGRNHTL VSTEGGNVYA TGGNNEGQLG |
| 121 | LGDTEERNTF HVISFFTSEH KIKQLSAGSN TSAALTEDGR LFMWGDNSEG QIGLKNVSNV |
| 181 | CVPQQVTIGK PVSWISCGYY HSAFVTTDGE LYVFGEPENG KLGLPNQLLG NHRTPQLVSE |
| 241 | IPEKVIQVAC GGEHTVVLTE NAVYTFGLGQ FGQLGLGTFL FETSEPKVIE NIRDQTISYI |
| 301 | SCGENHTALI TDIGLMYTFG DGRHGKLGLG LENFTNHFIP TLCSNFLRFI VKLVACGGCH |
| 361 | MVVFAAPHRG VAKEIEFDEI NDTCLSVATF LPYSSLTSGN VLQRTLSARM RRRERERSPD |
| 421 | SFSMRRTLPP IEGTLGLSAC FLPNSVFPRC SERNLQESVL SEQDLMQPEE PDYLLDEMTK |
| 481 | EAEIDNSSTV ESLGETTDIL NMTHIMSLNS NEKSLKLSPV QKQKKQQTIG ELTQDTALTE |
| 541 | NDDSDEYEEM SEMKEGKACK QHVSQGIFMT QPATTIEAFS DEEVEIPEEK EGAEDSKGNG |
| 601 | IEEQEVEANE ENVKVHGGRK EKTEILSDDL TDKAEVSEGK AKSVGEAEDG PEGRGDGTCE |
| 661 | EGSSGAEHWQ DEEREKGEKD KGRGEMERPG EGEKELAEKE EWKKRDGEEQ EQKEREQGHQ |
| 721 | KERNQEMEEG GEEEHGEGEE EEGDREEEEE KEGEGKEEGE GEEVEGEREK EEGERKKEER |
| 781 | AGKEEKGEEE GDQGEGEEEE TEGRGEEKEE GGEVEGGEVE EGKGEREEEE EEGEGEEEEG |
| 841 | EGEEEEGEGE EEEGEGKGEE EGEEGEGEEE GEEGEGEGEE EEGEGEGEEE GEGEGEEEEG |
| 901 | EGEGEEEGEG EGEEEEGEGK GEEEGEEGEG EGEEEEGEGE GEDGEGEGEE EEGEWEGEEE |
| 961 | EGEGEGEEEG EGEGEEGEGE GEEEEGEGEG EEEEGEEEGE EEGEGEEEGE GEGEEEEEGE |
| 1021 | VEGEVEGEEG EGEGEEEEGE EEGEEREKEG EGEENRRNRE EEEEEEGKYQ ETGEEENERQ |
| 1081 | DGEEYKKVSK IKGSVKYGKH KTYQKKSVTN TQGNGKEQRS KMPVQSKRLL KNGPSGSKKF |
| 1141 | WNNVLPHYLE LK. |

133. The method of claim 131 or 132, wherein the sequence encoding RPGR^{ORF15} comprises or consists of a nucleotide sequence of:

| (SEQ ID NO: 80) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3081 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa. |

133. The method of any one of claims 128-132, wherein the sequence encoding the polyA signal comprises a bovine growth hormone (BGH) polyA sequence.

134. The method of claim 133, wherein the sequence encoding the BGH polyA signal comprises a nucleotide sequence of:

| (SEQ ID NO: 83) | |
|---|---|
| 1 | cgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg. |

135. The method of any one of claims 128-134, wherein the plasmid vector comprising an exogenous sequence further comprises a sequence encoding a 5′ inverted terminal repeat (ITR) and a sequence encoding a 3′ ITR.

136. The method of any one of claims 128-134, wherein the sequence encoding the 5′ ITR and the sequence encoding the 3′ ITR are derived from a 5′ITR sequence and a 3′ ITR sequence of an AAV of serotype 2 (AAV2).

137. The method of any one of claims 128-136, wherein the sequence encoding the 5′ ITR and the sequence encoding the 3′ ITR comprise sequences that are identical to a sequence of a 5′ITR and a sequence of a 3′ ITR of an AAV2.

138. The method of any one of claims 128-136, wherein the sequence encoding the 5′ ITR comprises or consists of the nucleotide sequence of:
(SEQ ID NO: 34)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT.

139. The method of any one of claim 128-136 or 138, wherein the sequence encoding the 3′ ITR comprises or consists of the nucleotide sequence of:
(SEQ ID NO: 35)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

140. The method of any one of claims 128-139, wherein the exogenous sequence further comprises a sequence encoding a Kozak sequence, optionally wherein the Kozak sequence comprises the nucleotide sequence of GGCCACCATG (SEQ ID NO: 73).

141. The method of any one of claims 128-140, wherein the exogenous sequence comprises the sequence of:

| (SEQ ID NO: 74) | |
|---|---|
| 1 | CTGCGCGCTC GCTCGCTCAC TGAGGCCGCC CGGGCGTCGG GCGACCTTTG GTCGCCCGGC |
| 61 | CTCAGTGAGC GAGCGAGCGC GCAGAGAGGG AGTGGCCAAC TCCATCACTA GGGGTTCCTG |
| 121 | CGGCAATTCA GTCGATAACT ATAACGGTCC TAAGGTAGCG ATTTAAATAC GCGCTCTCTT |
| 181 | AAGGTAGCCC CGGGACGCGT CAATTGGGGC CCCAGAAGCC TGGTGGTTGT TTGTCCTTCT |
| 241 | CAGGGGAAAA GTGAGGCGGC CCCTTGGAGG AAGGGGCCGG GCAGAATGAT CTAATCGGAT |
| 301 | TCCAAGCAGC TCAGGGGATT GTCTTTTTCT AGCACCTTCT TGCCACTCCT AAGCGTCCTC |
| 361 | CGTGACCCCG GCTGGGATTT AGCCTGGTGC TGTGTCAGCC CCGGGGCCAC CATGAGAGAG |
| 421 | CCAGAGGAGC TGATGCCAGA CAGTGGAGCA GTGTTTACAT TCGGAAAATC TAAGTTCGCT |
| 481 | GAAAATAACC CAGGAAAGTT CTGGTTTAAA AACGACGTGC CCGTCCACCT GTCTTGTGGC |
| 541 | GATGAGCATA GTGCCGTGGT CACTGGGAAC AATAAGCTGT ACATGTTCGG GTCCAACAAC |
| 601 | TGGGGACAGC TGGGGCTGGG ATCCAAATCT GCTATCTCTA AGCCAACCTG CGTGAAGGCA |
| 661 | CTGAAACCCG AGAAGGTCAA ACTGGCCGCT TGTGGCAGAA ACCACACTCT GGTGAGCACC |
| 721 | GAGGGCGGGA ATGTCTATGC CACCGGAGGC AACAATGAGG GACAGCTGGG ACTGGGGGAC |
| 781 | ACTGAGGAAA GGAATACCTT TCACGTGATC TCCTTCTTTA CATCTGAGCA TAAGATCAAG |
| 841 | CAGCTGAGCG CTGGCTCCAA CACATCTGCA GCCCTGACTG AGGACGGGCG CCTGTTCATG |
| 901 | TGGGGAGATA ATTCAGAGGG CCAGATTGGG CTGAAAAACG TGAGCAATGT GTGCGTCCCT |
| 961 | CAGCAGGTGA CCATCGGAAA GCCAGTCAGT TGGATTTCAT GTGGCTACTA TCATAGCGCC |
| 1021 | TTCGTGACCA CAGATGGCGA GCTGTACGTC TTTGGGGAGC CCGAAAACGG AAAACTGGGC |
| 1081 | CTGCCTAACC AGCTGCTGGG CAATCACCGG ACACCCCAGC TGGTGTCCGA GATCCCTGAA |
| 1141 | AAAGTGATCC AGGTCGCCTG CGGGGGAGAG CATACAGTGG TCCTGACTGA GAATGCTGTG |
| 1201 | TATACCTTCG GACTGGGCCA GTTTGGCCAG CTGGGGCTGG GAACCTTCCT GTTTGAGACA |
| 1261 | TCCGAACCAA AAGTGATCGA GAACATTCGC GACCAGACTA TCAGCTACAT TTCCTGCGGA |
| 1321 | GAGAATCACA CCGCACTGAT CACAGACATT GGCCTGATGT ATACCTTTGG CGATGGACGA |
| 1381 | CACGGGAAGC TGGGACTGGG ACTGGAGAAC TTCACTAATC ATTTTATCCC CACCCTGTGT |
| 1441 | TCTAACTTCC TGCGGTTCAT CGTGAAACTG GTCGCTTGCG GCGGGTGTCA CATGGTGGTC |
| 1501 | TTCGCTGCAC CTCATAGGGG CGTGGCTAAG GAGATCGAAT TTGACGAGAT TAACGATACA |
| 1561 | TGCCTGAGCG TGGCAACTTT CCTGCCATAC AGCTCCCTGA CTTCTGGCAA TGTGCTGCAG |
| 1621 | AGAACCCTGA GTGCAAGGAT GCGGAGAAGG GAGAGGGAAC GCTCTCCTGA CAGTTTCTCA |
| 1681 | ATGCGACGAA CCCTGCCACC TATCGAGGGA ACACTGGGAC TGAGTGCCTG CTTCCTGCCT |
| 1741 | AACTCAGTGT TTCCACGATG TAGCGAGCGG AATCTGCAGG AGTCTGTCCT GAGTGAGCAG |
| 1801 | GATCTGATGC AGCCAGAGGA ACCCGACTAC CTGCTGGATG AGATGACCAA GGAGGCCGAA |
| 1861 | ATCGACAACT CTAGTACAGT GGAGTCCCTG GGCGAGACTA CCGATATCCT GAATATGACA |
| 1921 | CACATTATGT CACTGAACAG CAATGAGAAG AGTCTGAAAC TGTCACCAGT GCAGAAGCAG |
| 1981 | AAGAAACAGC AGACTATTGG CGAGCTGACT CAGGACACCG CCCTGACAGA GAACGACGAT |
| 2041 | AGCGATGAGT ATGAGGAAAT GTCCGAGATG AAGGAAGGCA AAGCTTGTAA GCAGCATGTC |
| 2101 | AGTCAGGGGA TCTTCATGAC ACAGCCAGCC ACAACTATTG AGGCTTTTTC AGACGAGGAA |
| 2161 | GTGGAGATCC CCGAGGAAAA AGAGGGCGCA GAAGATTCCA AGGGGAATGG AATTGAGGAA |
| 2221 | CAGGAGGTGG AAGCCAACGA GGAAAATGTG AAAGTCCACG GAGGCAGGAA GGAGAAAACA |
| 2281 | GAAATCCTGT CTGACGATCT GACTGACAAG GCCGAGGTGT CCGAAGGCAA GGCAAAATCT |
| 2341 | GTCGGAGAGG CAGAAGACGG ACCAGAGGGA CGAGGGGATG GAACCTGCGA GGAAGGCTCA |
| 2401 | AGCGGGGCTG AGCATTGGCA GGACGAGGAA CGAGAGAAGG GCGAAAAGGA TAAAGGCCGC |
| 2461 | GGGGAGATGG AACGACCTGG AGAGGGCGAA AAAGAGCTGG CAGAGAAGGA GGAATGGAAG |
| 2521 | AAAAGGGACG GCGAGGAACA GGAGCAGAAA GAAAGGGAGC AGGGCCACCA GAAGGAGCGC |
| 2581 | AACCAGGAGA TGGAAGAGGG CGGCGAGGAA GAGCATGGCG AGGGAGAAGA GGAAGAGGGC |
| 2641 | GATAGAGAAG AGGAAGAGGA AAAAGAAGGC GAAGGGAAGG AGGAAGGAGA GGGCGAGGAA |
| 2701 | GTGGAAGGCG AGAGGGAAAA GGAGGAAGGA GAACGGAAGA AAGAGGAAAG AGCCGGCAAA |
| 2761 | GAGGAAAAGG GCGAGGAAGA GGGCGATCAG GGCGAAGGCG AGGAGGAAGA GACCGAGGGC |
| 2821 | CGCGGGGAAG AGAAAGAGGA GGGAGGAGAG GTGGAGGGCG GAGAGGTCGA AGAGGGAAAG |
| 2881 | GGCGAGCGCG AAGAGGAAGA GGAAGAGGGC GAGGGCGAGG AAGAAGAGGG CGAGGGGGAA |
| 2941 | GAAGAGGAGG GAGAGGGCGA AGAGGAAGAG GGGGAGGGAA AGGGCGAAGA GGAAGGAGAG |
| 3001 | GAAGGGGAGG GAGAGGAAGA GGGGGAGGAG GGCGAGGGGG AAGGCGAGGA GGAAGAAGGA |
| 3061 | GAGGGGGAAG GCGAAGAGGA AGGCGAGGGG GAAGGAGAGG AGGAAGAAGG GGAAGGCGAA |
| 3121 | GGCGAAGAGG AGGGAGAAGG AGAGGGGGAG GAAGAGGAAG GAGAAGGGAA GGGCGAGGAG |
| 3181 | GAAGGCGAAG AGGGAGAGGG GGAAGGCGAG GAAGAGGAAG GCGAGGGCGA AGGAGAGGAC |
| 3241 | GGCGAGGGCG AGGGAGAAGA GGAGGAAGGG GAATGGGAAG GCGAAGAAGA GGAAGGCGAA |
| 3301 | GGCGAAGGCG AAGAAGAGGG CGAAGGGGAG GGCGAGGAGG GCGAAGGCGA AGGGGAGGAA |
| 3361 | GAGGAAGGCG AAGGAGAAGG CGAGGAAGAA GAGGGAGAGG AGGAAGGCGA GGAGGAAGGA |
| 3421 | GAGGGGGAGG AGGAGGGAGA AGGCGAGGGC GAAGAAGAAG AAGAGGGAGA AGTGGAGGGC |
| 3481 | GAAGTCGAGG GGGAGGAGGG AGAAGGGGAA GGGGAGGAAG AAGAGGGCGA AGAAGAAGGC |
| 3541 | GAGGAAAGAG AAAAAGAGGG AGAAGGCGAG GAAAACCGGA GAAATAGGGA AGAGGAGGAA |
| 3601 | GAGGAAGAGG GAAAGTACCA GGAGACAGGC GAAGAGGAAA ACGAGCGGCA GGATGGCGAG |
| 3661 | GAATATAAGA AAGTGAGCAA GATCAAAGGA TCCGTCAAGT ACGGCAAGCA CAAAACCTAT |
| 3721 | CAGAAGAAAA GCGTGACCAA CACACAGGGG AATGGAAAAG AGCAGAGGAG TAAGATGCCT |
| 3781 | GTGCAGTCAA AACGGCTGCT GAAGAATGGC CCATCTGGAA GTAAAAAATT CTGGAACAAT |
| 3841 | GTGCTGCCCC ACTATCTGGA ACTGAAATAA GAGCTCCTCG AGGCGGCCCG CTCGAGTCTA |
| 3901 | GAGGGCCCTT CGAAGGTAAG CCTATCCCTA ACCCTCTCCT CGGTCTCGAT TCTACGCGTA |
| 3961 | CCGGTCATCA TCACCATCAC CATTGAGTTT AAACCCGCTG ATCAGCCTCG ACTGTGCCTT |
| 4021 | CTAGTTGCCA GCCATCTGTT GTTTGCCCCT CCCCCGTGCC TTCCTTGACC CTGGAAGGTG |
| 4081 | CCACTCCCAC TGTCCTTTCC TAATAAAATG AGGAAATTGC ATCGCATTGT CTGAGTAGGT |
| 4141 | GTCATTCTAT TCTGGGGGGT GGGGTGGGGC AGGACAGCAA GGGGGAGGAT TGGGAAGACA |
| 4201 | ATAGCAGGCA TGCTGGGGAT GCGGTGGGCT CTATGGCTTC TGAGGCGGAA AGAACCAGAT |
| 4261 | CCTCTCTTAA GGTAGCATCG AGATTTAAAT TAGGGATAAC AGGGTAATGG CGCGGGCCGC |
| 4321 | AGGAACCCCT AGTGATGGAG TTGGCCACTC CCTCTCTGCG CGCTCGCTCG CTCACTGAGG |
| 4381 | CCGGGCGACC AAAGGTCGCC CGACGCCCGG GCTTTGCCCG GGCGGCCTCA GTGAGCGAGC |
| 4441 | GAGCGCGCAG. |

142. The method of any one of claims 125-127, wherein the exogenous sequence comprises a sequence encoding an ATP Binding Cassette, Subfamily Member 4 (ABCA4) protein or a portion thereof.

143. The method of claim 142, wherein the exogenous sequence comprises a 5′ sequence encoding an ABCA4 protein or a portion thereof.

144. The method of claim 142, wherein the exogenous sequence comprises a 3′ sequence encoding an ABCA4 protein or a portion thereof.

145. The method of claim 142, wherein the exogenous sequence further comprises a promoter sequence.

146. The method of claim 145, wherein the exogenous sequence comprises a rhodopsin kinase (RK) promoter sequence, optionally a GRK1 promoter sequence.

147. The method of claim 146, wherein the GRK1 promoter sequence comprises or consists of:

| (SEQ ID NO: 5) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

148. The method of claim 145, wherein the exogenous sequence comprises a chicken beta-actin (CBA) promoter sequence.

149. The method of claim 148, wherein the CBA promoter sequence comprises or consists of:

| (SEQ ID NO: 16) | |
|---|---|
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCGG GAGTCGCTGC GCGCTGCCTT |
| 301 | CGCCCCGTGC CCCGCTCCGC CGCCGCCTCG CGCCGCCCGC CCCGGCTCTG ACTGACCGCG |
| 361 | TTACTCCCAC AG |
| or | |
| (SEQ ID NO: 24) | |
| 1 | GTCGAGGTGA GCCCCACGTT CTGCTTCACT CTCCCCATCT CCCCCCCCTC CCCACCCCCA |
| 61 | ATTTTGTATT TATTTATTTT TTAATTATTT TGTGCAGCGA TGGGGGCGGG GGGGGGGGGG |
| 121 | GGGCGCGCGC CAGGCGGGGC GGGGCGGGGC GAGGGGCGGG GCGGGGCGAG GCGGAGAGGT |
| 181 | GCGGCGGCAG CCAATCAGAG CGGCGCGCTC CGAAAGTTTC CTTTTATGGC GAGGCGGCGG |
| 241 | CGGCGGCGGC CCTATAAAAA GCGAAGCGCG CGGCGGGCG. |

150. The method of claim 142, wherein the exogenous sequence comprises a CMV.CBA promoter sequence, a CBA.RBG promoter sequence, or a CBA.InEx promoter sequence.

151. The method of any one of claims 142-150, wherein the sequence encoding the ABCA4 is a human ABCA4 sequence or a variant thereof.

152. The method of claim 151, wherein the sequence encoding ABCA4 comprises a 5′ nucleotide sequence comprising nucleotides 1-3701 or 1-4326 of SEQ ID NO: 2 or SEQ ID NO: 1.

153. The method of claim 151, wherein the sequence encoding ABCA4 comprises a 3′ nucleotide sequence comprising nucleotides 3154-6822, 3196-6822, 3494-6822, 3603-6822, 3653-6822, 3678-6822, 3702-6822 or 3494-6822 of SEQ ID NO: 2 or SEQ ID NO: 1.

154. The method of any of claims 142-153, wherein the plasmid vector comprising an exogenous sequence further comprises a sequence encoding a 5′ inverted terminal repeat (ITR) and a sequence encoding a 3′ ITR.

155. The method of claim 154, wherein the sequence encoding the 5′ ITR and the sequence encoding the 3′ ITR are derived from a 5′ITR sequence and a 3′ ITR sequence of an AAV of serotype 2 (AAV2) or a variant thereof.

156. The method of claim 154, wherein the 5′ ITR comprises or consists of:
(SEQ ID NO: 36)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT.

157. The method of any of claims 142-156, wherein the exogenous sequence comprises a 3′ ITR.

158. The method of claim 157, wherein the 5′ ITR comprises or consists of:
(SEQ ID NO: 37)
AGGAACCCCTAGTGATGGAGTTGGCCACTCCCTCTCTGCGCGCTCGCTCG
CTCACTGAGGCCGGGCGACCAAAGGTCGCCCGACGCCCGGGCGGCCTCAG
TGAGCGAGCGAGCGCGCAGAG.

159. The method of any one of claims 125-158, wherein the plasmid vector comprising an exogenous sequence, the helper plasmid vector or the plasmid vector comprising the sequence encoding a viral Rep protein and a viral Cap protein further comprises a sequence encoding a selection marker.

160. The method of any one of claims 125-159, wherein the sequence encoding the viral Rep protein and the sequence encoding the viral Cap protein comprise sequences isolated or derived from AAV serotype 8 (AAV8) viral Rep protein and viral Cap protein sequences.


SEQUENCE LISTING (86 sequences)

SEQ ID NO: 1 - DNA, 7326 bp, Homo sapiens
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 2 - DNA, 7326 bp, Homo sapiens
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 3 - DNA, 4464 bp, Artificial Sequence
  Made in Lab - upstream vector sequence, comprising 5′ ITR, promoter, CDS, 3′ ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtaatggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 4 - DNA, 4581 bp, Artificial Sequence
  Made in Lab - downstream vector sequence, comprising 5′ ITR, CDS, post-transcriptional response element, poly-adenylation sequence, 3′ ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggatttaaattagggataacagggtaatggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 5 - DNA, 199 bp, Homo sapiens
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggg

SEQ ID NO: 6 - DNA, 186 bp, Artificial Sequence
  Made in Lab - UTR including CBA and RBG fragments
gtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgg

SEQ ID NO: 7 - DNA, 593 bp, Woodchuck hepatitis virus
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 8 - DNA, 269 bp, Bos taurus
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 9 - DNA, 4087 bp, Artificial Sequence
  Made in Lab - partial upstream vector sequence, comprising promoter, CDS
ggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcc

SEQ ID NO: 10 - DNA, 4203 bp, Artificial Sequence
  Made in Lab - partial downstream vector sequence, comprising CDS, post transcriptional response element, poly-adenylation sequence
acatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 11 - DNA, 6822 bp, Homo sapiens
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 12 - protein, 737 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRN

SEQ ID NO: 13 - DNA, 123 bp, Artificial Sequence
  Made in Lab
gtgccgcagggggacggctgccttcgggggggacggggcagggcggggttcggcttctggcgtgtgaccggcggctctagagcctctgctaaccatgttcatgccttcttctttttcctacag

SEQ ID NO: 14 - DNA, 53 bp, Oryctolagus cuniculus
ctcctgggcaacgtgctggttattgtgctgtctcatcattttggcaaagaatt

SEQ ID NO: 15 - DNA, 235 bp, Human betaherpesvirus 5
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtca

SEQ ID NO: 16 - DNA, 372 bp, Gallus gallus
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacag

SEQ ID NO: 17 - DNA, 7496 bp, Artificial Sequence
  Made in Lab - CMVCBA.In.GFP.pA vector
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 18 - DNA, 7321 bp, Artificial Sequence
  Made in Lab - CMVCBA.GFP.pA vector
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 19 - DNA, 7483 bp, Artificial Sequence
  Made in Lab - CBA.IntEx.GFP.pA vector
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 20 - DNA, 7728 bp, Artificial Sequence
  Made in Lab - CAG.GFP.pA vector
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattctag

SEQ ID NO: 21 - DNA, 10070 bp, Artificial Sequence
  Made in Lab - AAV.5′CMVCBA.In.ABCA4.WPRE.kan vector
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 22 - DNA, 9895 bp, Artificial Sequence
  Made in Lab - AAV.5′CMVCBA.ABCA4.WPRE.kan vector
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 23 - DNA, 10057 bp, Artificial Sequence
  Made in Lab - AAV.5′CBA.IntEx.ABCA4.WPRE.kan vector
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 24 - DNA, 279 bp, Gallus gallus
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 25 - DNA, 263 bp, Bos taurus
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccag

SEQ ID NO: 26 - DNA, 4464 bp, Artificial Sequence; identical to SEQ ID NO: 3
  Made in Lab - pAAV.RK.5′ABCA4.kan

SEQ ID NO: 27 - DNA, 145 bp, Adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 28 - DNA, 199 bp, Homo sapiens; identical to SEQ ID NO: 5

SEQ ID NO: 29 - DNA, 3703 bp, Homo sapiens
catgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcca

SEQ ID NO: 30 - DNA, 145 bp, Artificial Sequence
  Recombinant synthesis
  misc_feature (1)..(145): 3′ ITR
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 31 - DNA, 3334 bp, Homo sapiens
aaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 32 - DNA, 593 bp, Woodchuck hepatitis viurs; identical to SEQ ID NO: 7

SEQ ID NO: 33 - DNA, 269 bp, Adeno-associated viurs 2; identical to SEQ ID NO: 8

SEQ ID NO: 34 - DNA, 119 bp, Adeno-associated virus 2
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 35 - DNA, 130 bp, Adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 130 bp, Adeno-associated virus 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 37 - DNA, 121 bp, Adeno associated virus 2
aggaacccctagtgatggagttggccactccctctctgcgcgctcgctcgctcactgaggccgggcgaccaaaggtcgcccgacgcccgggcggcctcagtgagcgagcgagcgcgcagag

SEQ ID NO: 38 - DNA, 270 bp, Bos taurus
tcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 39 - DNA, 188 bp, Oryctolagus cuniculus
agccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacca

SEQ ID NO: 40 - protein, 2273 aa, Homo sapiens [sequence omitted]

SEQ ID NO: 41 - DNA, 9819 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggatttaaattagggataacagggtaatggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 42 - DNA, 120 bp, Artificial Sequence
  Recombinant synthesis
gctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 43 - DNA, 3334 bp, Artificial Sequence; identical to SEQ ID NO: 31
  Recombinant synthesis

SEQ ID NO: 44 - DNA, 593 bp, Artificial Sequence; identical to SEQ ID NO: 7
  Recombinant synthesis

SEQ ID NO: 45 - DNA, 174 bp, Artificial Sequence
  Recombinant synthesis
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggg

SEQ ID NO: 46 - DNA, 121 bp, Artificial Sequence; identical to SEQ ID NO: 37
  Recombinant synthesis

SEQ ID NO: 47 - DNA, 9786 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 48 - DNA, 115 bp, Artificial Sequence
  Recombinant synthesis
  5′ ITR (1)..(115)
ctcactgaggccgcccgggcaaagcccgggcgtcgggcgacctttggtcgcccggcctcagtgagcgagcgagcgcgcagagagggagtggccaactccatcactaggggttcct

SEQ ID NO: 49 - DNA, 278 bp, Artificial Sequence
  Recombinant synthesis
  CBA (1)..(278)
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggc

SEQ ID NO: 50 - DNA, 123 bp, Artificial Sequence; identical to SEQ ID NO: 13
  Recombinant synthesis
  Intron (1)..(123)

SEQ ID NO: 51 - DNA, 40 bp, Artificial Sequence
  Recombinant synthesis
  exon (1)..(40)
ctcctgggcaacgtgctggttattgtgctgtctcatcatt

SEQ ID NO: 52 - DNA, 3702 bp, Artificial Sequence
  Recombinant synthesis
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcca

SEQ ID NO: 53 - DNA, 121 bp, Artificial Sequence; identical to SEQ ID NO: 37
  Recombinant synthesis
  3′ ITR (1)..(121)

SEQ ID NO: 54 - DNA, 10055 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtacccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgatatcgaattcctgcagcccggcaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 55 - DNA, 115 bp, Artificial Sequence; identical to SEQ ID NO: 48
  Recombinant synthesis

SEQ ID NO: 56 - DNA, 278 bp, Artificial Sequence; identical to SEQ ID NO: 49
  Recombinant synthesis

SEQ ID NO: 57 - DNA, 173 bp, Artificial Sequence
  Recombinant synthesis
ccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 58 - DNA, 3702 bp, Artificial Sequence; identical to SEQ ID NO: 52
  Recombinant synthesis

SEQ ID NO: 59 - DNA, 121 bp, Artificial Sequence; identical to SEQ ID NO: 37
  Recombinant synthesis

SEQ ID NO: 60 - DNA, 9992 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtaccctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 61 - DNA, 115 bp, Artificial Sequence; identical to SEQ ID NO: 48
  Recombinant synthesis

SEQ ID NO: 62 - DNA, 235 bp, Artificial Sequence; identical to SEQ ID NO: 15
  Recombinant synthesis
  CMV Enhancer (1)..(235)

SEQ ID NO: 63 - DNA, 278 bp, Artificial Sequence; identical to SEQ ID NO: 49
  Recombinant synthesis
  CBA promoter (1)..(278)

SEQ ID NO: 64 - DNA, 3702 bp, Artificial Sequence; identical to SEQ ID NO: 52
  Recombinant synthesis

SEQ ID NO: 65 - DNA, 121 bp, Artificial Sequence; identical to SEQ ID NO: 37
  Recombinant synthesis

SEQ ID NO: 66 - DNA, 9702 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtggtggcgcgggccgcaggaacccctagtgatggagttggccactccctctctgcgcgctcgctcgctcactgaggccgggcgaccaaaggtcgcccgacgcccgggcggcctcagtgagcgagcgagcgcgcagagctagaattaattccgtgtattctatagtgtcacctaaatcgtatgtgtatgatacataaggttatgtattaattgtagccgcgttctaacgacaatatgtacaagcctaattgtgtagcatctggcttagcggccgcctaccgtcaaacagtcaatcccgttctacgccatttgacacataacgcccgggataacagagctgaatttgacggactacgatattgcttatgtgccaccaatcaacagttaacgaacacgtggcggcgcggaacgcctccggccaggccgcgcgcttcgcatatttacttcgagcagtgtaggtgtgacaacgtagcatgcagccacatccctagcttgaaccggagataaaggtctacgcgcgcgacgtccacattcacacggttcagattcctggtgctacccaaaacaaagtccataggtttttcattgggactacggcgcgaagctaagtggtttcacacctacaagggaaacatgcccaaactatgaggacaacatcgtccgcagaaacaatcggccgcgataggggttgcacgttgtcagatgaaagagccacactcggggagcagtccgcggacgccacctcgtgcaacttcggctaaccatataatctaaaaaagttgaggtttgcagttgtcggggcgagatcaaacccaagtatatagtcctgtccggagccttagttcacgtactcgcgacccttgaaagcgcgtcaagcttatcgctcactgactagctcaatgtgtggcaatctaagtaggaggtctgtcgcaaggcaaaaatgctaattattggtagcaagcttagataaggtggagggattgcacaattcagaaggcgtcttctctgctacacccgagcggggtgctttatcaaggggaagcttgatgtcccacgggatgaacgagagcctccatggcatctcacgacctacttaacttcgggggatgggtagaagttagctgaacatacaaatgggaataggattgtgccctcggacgagactgaacggatcgcagtcaacccgcgcaaagtttacatattaattcttacggcgtgtcagagaggcaatggcttgacttgtggtggatcacagtttgtgagtaacggcaagatgcggtaaacactgtaatgcgagcttcattgactcggcttaaagttcctggtaccataatgaatacacggtggttagttgtcaattgcttgtgcaccgccgcaccttgcggtcctcggtccagcctgcgcagggtataaatgaagcacgtcccacccagactgttccatcgtacctccaaatacggattcaacctggcgtctatttccagatatgggccctaggggtgatagactcccaagtctaaggactaccatgggatatgtttcacgtatccaaaaagtaaccataatactgcgtttccgttcacccaagtgaggatgttgcctttgtactggtttcatagtcctgccgtaccaggcgtcttccttagccggcgctacttccagcccggaactgtcttgtttctcgatgtgagacccttgtcagccgcccgcggtggtgcacgtaaaagccgattggagtattaagtatttacaactccgaatcttaagagccctgctctagtttggattcatatatcagcataggcttcgcaacctagtgaatgagcggtacgaactttcgcggagtgcgaaaagcgaccgagcaatcgagatacgtaccgttagattcacgctccagacagcactctgagtctttgatttataaccatcgaaggaatcgacttcacgtccctagcgtgttgagtcatccgcagaagagacgatgagggctcgccccccgaaatagttctgcttcaaactataggctgccctacttggtctccgaggtactatggggtcctcgacggttcgaggcccccaacccatgttcaatcagctcgtatgtctaccctcgagctaacacaggaaccagctgagacttgcctggcgtcacttgggcacgttccatatacataatgaagtacgccgcagggtctctccgttaccgaactgtgctcgacctaaagtccggtacccatcggcgtcctgtcacatttgtggcattaggtatgaactaactctggggggcttctacgaccatggtaaaagttttgtgctgccagacaactgttaataaacatgtcgctgcgtagaacgccaagaaccagctgggatgagtgccttatttaccccgcgcgaggtgggtctgagtaggtagcatcgaggtttacgcctaagttggaccgcaaatataggccctttgccgggatccccactatctgtgaattgtgaaacccgttggcaccctgtacaaagtgcatagctacatcattggtaacaagacgtaaacggaggttcgctcactcccacttcggaaagataaccggggaactaggagggtatggtgcgcgcatggaaagggccgggaagtaactctggccttcacggaacgataagttacaatttgggaacagtcggagagcgccactacgtgcttttttggcttacctcatatctcgtagttggtgagggttaaaattcgcgggagaagatccagcctaagtatatggttacatcgcggccgcctgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaag

SEQ ID NO: 67 - DNA, 115 bp, Artificial Sequence; identical to SEQ ID NO: 48
  Recombinant synthesis

SEQ ID NO: 68 - DNA, 199 bp, Artificial Sequence; identical to SEQ ID NO: 5
  Recombinant synthesis

SEQ ID NO: 69 - DNA, 3702 bp, Artificial Sequence; identical to SEQ ID NO: 52
  Recombinant synthesis

SEQ ID NO: 70 - DNA, 121 bp, Artificial Sequence; identical to SEQ ID NO: 37
  Recombinant synthesis

SEQ ID NO: 71 - DNA, 119 bp, Adeno-associated virus 2; identical to SEQ ID NO: 34

SEQ ID NO: 72 - DNA, 130 bp, Adeno-associated virus 2; identical to SEQ ID NO: 35

SEQ ID NO: 73 - DNA, 10 bp, Artificial Sequence
  Consensus Kozak sequence
ggccaccatg

SEQ ID NO: 74 - DNA, 4450 bp, Artificial Sequence
  Recombinant synthesis
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggggccaccatgagagagccagaggagctgatgccagacagtggagcagtgtttacattcggaaaatctaagttcgctgaaaataacccaggaaagttctggtttaaaaacgacgtgcccgtccacctgtcttgtggcgatgagcatagtgccgtggtcactgggaacaataagctgtacatgttcgggtccaacaactggggacagctggggctgggatccaaatctgctatctctaagccaacctgcgtgaaggcactgaaacccgagaaggtcaaactggccgcttgtggcagaaaccacactctggtgagcaccgagggcgggaatgtctatgccaccggaggcaacaatgagggacagctgggactgggggacactgaggaaaggaatacctttcacgtgatctccttctttacatctgagcataagatcaagcagctgagcgctggctccaacacatctgcagccctgactgaggacgggcgcctgttcatgtggggagataattcagagggccagattgggctgaaaaacgtgagcaatgtgtgcgtccctcagcaggtgaccatcggaaagccagtcagttggatttcatgtggctactatcatagcgccttcgtgaccacagatggcgagctgtacgtctttggggagcccgaaaacggaaaactgggcctgcctaaccagctgctgggcaatcaccggacaccccagctggtgtccgagatccctgaaaaagtgatccaggtcgcctgcgggggagagcatacagtggtcctgactgagaatgctgtgtataccttcggactgggccagtttggccagctggggctgggaaccttcctgtttgagacatccgaaccaaaagtgatcgagaacattcgcgaccagactatcagctacatttcctgcggagagaatcacaccgcactgatcacagacattggcctgatgtatacctttggcgatggacgacacgggaagctgggactgggactggagaacttcactaatcattttatccccaccctgtgttctaacttcctgcggttcatcgtgaaactggtcgcttgcggcgggtgtcacatggtggtcttcgctgcacctcataggggcgtggctaaggagatcgaatttgacgagattaacgatacatgcctgagcgtggcaactttcctgccatacagctccctgacttctggcaatgtgctgcagagaaccctgagtgcaaggatgcggagaagggagagggaacgctctcctgacagtttctcaatgcgacgaaccctgccacctatcgagggaacactgggactgagtgcctgcttcctgcctaactcagtgtttccacgatgtagcgagcggaatctgcaggagtctgtcctgagtgagcaggatctgatgcagccagaggaacccgactacctgctggatgagatgaccaaggaggccgaaatcgacaactctagtacagtggagtccctgggcgagactaccgatatcctgaatatgacacacattatgtcactgaacagcaatgagaagagtctgaaactgtcaccagtgcagaagcagaagaaacagcagactattggcgagctgactcaggacaccgccctgacagagaacgacgatagcgatgagtatgaggaaatgtccgagatgaaggaaggcaaagcttgtaagcagcatgtcagtcaggggatcttcatgacacagccagccacaactattgaggctttttcagacgaggaagtggagatccccgaggaaaaagagggcgcagaagattccaaggggaatggaattgaggaacaggaggtggaagccaacgaggaaaatgtgaaagtccacggaggcaggaaggagaaaacagaaatcctgtctgacgatctgactgacaaggccgaggtgtccgaaggcaaggcaaaatctgtcggagaggcagaagacggaccagagggacgaggggatggaacctgcgaggaaggctcaagcggggctgagcattggcaggacgaggaacgagagaagggcgaaaaggataaaggccgcggggagatggaacgacctggagagggcgaaaaagagctggcagagaaggaggaatggaagaaaagggacggcgaggaacaggagcagaaagaaagggagcagggccaccagaaggagcgcaaccaggagatggaagagggcggcgaggaagagcatggcgagggagaagaggaagagggcgatagagaagaggaagaggaaaaagaaggcgaagggaaggaggaaggagagggcgaggaagtggaaggcgagagggaaaaggaggaaggagaacggaagaaagaggaaagagccggcaaagaggaaaagggcgaggaagagggcgatcagggcgaaggcgaggaggaagagaccgagggccgcggggaagagaaagaggagggaggagaggtggagggcggagaggtcgaagagggaaagggcgagcgcgaagaggaagaggaagagggcgagggcgaggaagaagagggcgagggggaagaagaggagggagagggcgaagaggaagagggggagggaaagggcgaagaggaaggagaggaaggggagggagaggaagagggggaggagggcgagggggaaggcgaggaggaagaaggagagggggaaggcgaagaggaaggcgagggggaaggagaggaggaagaaggggaaggcgaaggcgaagaggagggagaaggagagggggaggaagaggaaggagaagggaagggcgaggaggaaggcgaagagggagagggggaaggcgaggaagaggaaggcgagggcgaaggagaggacggcgagggcgagggagaagaggaggaaggggaatgggaaggcgaagaagaggaaggcgaaggcgaaggcgaagaagagggcgaaggggagggcgaggagggcgaaggcgaaggggaggaagaggaaggcgaaggagaaggcgaggaagaagagggagaggaggaaggcgaggaggaaggagagggggaggaggagggagaaggcgagggcgaagaagaagaagagggagaagtggagggcgaagtcgagggggaggagggagaaggggaaggggaggaagaagagggcgaagaagaaggcgaggaaagagaaaaagagggagaaggcgaggaaaaccggagaaatagggaagaggaggaagaggaagagggaaagtaccaggagacaggcgaagaggaaaacgagcggcaggatggcgaggaatataagaaagtgagcaagatcaaaggatccgtcaagtacggcaagcacaaaacctatcagaagaaaagcgtgaccaacacacaggggaatggaaaagagcagaggagtaagatgcctgtgcagtcaaaacggctgctgaagaatggcccatctggaagtaaaaaattctggaacaatgtgctgccccactatctggaactgaaataagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 75 - DNA, 199 bp, Homo sapiens; identical to SEQ ID NO: 5

SEQ ID NO: 76 - DNA, 372 bp, Gallus gallus; identical to SEQ ID NO: 16

SEQ ID NO: 77 - DNA, 279 bp, Gallus gallus; identical to SEQ ID NO: 24

SEQ ID NO: 78 - protein, 1152 aa, Homo sapiens
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVEIPEEKEGAEDSKGNGIEEQEVEANEENVKVHGGRKEKTEILSDDLTDKAEVSEGKAKSVGEAEDGPEGRGDGTCEEGSSGAEHWQDEEREKGEKDKGRGEMERPGEGEKELAEKEEWKKRDGEEQEQKEREQGHQKERNQEMEEGGEEEHGEGEEEEGDREEEEEKEGEGKEEGEGEEVEGEREKEEGERKKEERAGKEEKGEEEGDQGEGEEEETEGRGEEKEEGGEVEGGEVEEGKGEREEEEEEGEGEEEEGEGEEEEGEGEEEEGEGKGEEEGEEGEGEEEGEEGEGEGEEEEGEGEGEEEGEGEGEEEEGEGEGEEEGEGEGEEEEGEGKGEEEGEEGEGEGEEEEGEGEGEDGEGEGEEEEGEWEGEEEEGEGEGEEEGEGEGEEGEGEGEEEEGEGEGEEEEGEEEGEEEGEGEEEGEGEGEEEEEGEVEGEVEGEEGEGEGEEEEGEEEGEEREKEGEGEENRRNREEEEEEEGKYQETGEEENERQDGEEYKKVSKIKGSVKYGKHKTYQKKSVTNTQGNGKEQRSKMPVQSKRLLKNGPSGSKKFWNNVLPHYLELK

SEQ ID NO: 79 - DNA, 3459 bp, Homo sapiens
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggttgcttgtggtggatgtcacatggtagtttttgctgctcctcatcgtggtgtggcaaaagaaattgaattcgatgaaataaatgatacttgcttatctgtggcgacttttctgccgtatagcagtttaacctcaggaaatgtactgcagaggactctatcagcacgtatgcggcgaagagagagggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaagaaacaacaaacaattggggaactgacgcaggatacagctcttactgaaaacgatgatagtgatgaatatgaagaaatgtcagaaatgaaagaagggaaagcatgtaaacaacatgtgtcacaagggattttcatgacgcagccagctacgactatcgaagcattttcagatgaggaagtagagatcccagaggagaaggaaggagcagaggattcaaaaggaaatggaatagaggagcaagaggtagaagcaaatgaggaaaatgtgaaggtgcatggaggaagaaaggagaaaacagagatcctatcagatgaccttacagacaaagcagaggtgagtgaaggcaaggcaaaatcagtgggagaagcagaggatgggcctgaaggtagaggggatggaacctgtgaggaaggtagttcaggagcagaacactggcaagatgaggagagggagaagggggagaaagacaagggtagaggagaaatggagaggccaggagagggagagaaggaactagcagagaaggaagaatggaagaagagggatggggaagagcaggagcaaaaggagagggagcagggccatcagaaggaaagaaaccaagagatggaggagggaggggaggaggagcatggagaaggagaagaagaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagaggaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaacaggaggaacagagaagaggaggaggaagaagaggggaagtatcaggagacaggcgaagaagagaatgaaaggcaggatggagaggagtacaaaaaagtgagcaaaataaaaggatctgtgaaatatggcaaacataaaacatatcaaaaaaagtcagttactaacacacagggaaatgggaaagagcagaggtccaaaatgccagtccagtcaaaacgacttttaaaaaacgggccatcaggttccaaaaagttctggaataatgtattaccacattacttggaattgaagtaa

SEQ ID NO: 80 - DNA, 3459 bp, Artificial Sequence
  Made in Lab - Codon optimized RPGR ORF15
atgagagagccagaggagctgatgccagacagtggagcagtgtttacattcggaaaatctaagttcgctgaaaataacccaggaaagttctggtttaaaaacgacgtgcccgtccacctgtcttgtggcgatgagcatagtgccgtggtcactgggaacaataagctgtacatgttcgggtccaacaactggggacagctggggctgggatccaaatctgctatctctaagccaacctgcgtgaaggcactgaaacccgagaaggtcaaactggccgcttgtggcagaaaccacactctggtgagcaccgagggcgggaatgtctatgccaccggaggcaacaatgagggacagctgggactgggggacactgaggaaaggaatacctttcacgtgatctccttctttacatctgagcataagatcaagcagctgagcgctggctccaacacatctgcagccctgactgaggacgggcgcctgttcatgtggggagataattcagagggccagattgggctgaaaaacgtgagcaatgtgtgcgtccctcagcaggtgaccatcggaaagccagtcagttggatttcatgtggctactatcatagcgccttcgtgaccacagatggcgagctgtacgtctttggggagcccgaaaacggaaaactgggcctgcctaaccagctgctgggcaatcaccggacaccccagctggtgtccgagatccctgaaaaagtgatccaggtcgcctgcgggggagagcatacagtggtcctgactgagaatgctgtgtataccttcggactgggccagtttggccagctggggctgggaaccttcctgtttgagacatccgaaccaaaagtgatcgagaacattcgcgaccagactatcagctacatttcctgcggagagaatcacaccgcactgatcacagacattggcctgatgtatacctttggcgatggacgacacgggaagctgggactgggactggagaacttcactaatcattttatccccaccctgtgttctaacttcctgcggttcatcgtgaaactggtcgcttgcggcgggtgtcacatggtggtcttcgctgcacctcataggggcgtggctaaggagatcgaatttgacgagattaacgatacatgcctgagcgtggcaactttcctgccatacagctccctgacttctggcaatgtgctgcagagaaccctgagtgcaaggatgcggagaagggagagggaacgctctcctgacagtttctcaatgcgacgaaccctgccacctatcgagggaacactgggactgagtgcctgcttcctgcctaactcagtgtttccacgatgtagcgagcggaatctgcaggagtctgtcctgagtgagcaggatctgatgcagccagaggaacccgactacctgctggatgagatgaccaaggaggccgaaatcgacaactctagtacagtggagtccctgggcgagactaccgatatcctgaatatgacacacattatgtcactgaacagcaatgagaagagtctgaaactgtcaccagtgcagaagcagaagaaacagcagactattggcgagctgactcaggacaccgccctgacagagaacgacgatagcgatgagtatgaggaaatgtccgagatgaaggaaggcaaagcttgtaagcagcatgtcagtcaggggatcttcatgacacagccagccacaactattgaggctttttcagacgaggaagtggagatccccgaggaaaaagagggcgcagaagattccaaggggaatggaattgaggaacaggaggtggaagccaacgaggaaaatgtgaaagtccacggaggcaggaaggagaaaacagaaatcctgtctgacgatctgactgacaaggccgaggtgtccgaaggcaaggcaaaatctgtcggagaggcagaagacggaccagagggacgaggggatggaacctgcgaggaaggctcaagcggggctgagcattggcaggacgaggaacgagagaagggcgaaaaggataaaggccgcggggagatggaacgacctggagagggcgaaaaagagctggcagagaaggaggaatggaagaaaagggacggcgaggaacaggagcagaaagaaagggagcagggccaccagaaggagcgcaaccaggagatggaagagggcggcgaggaagagcatggcgagggagaagaggaagagggcgatagagaagaggaagaggaaaaagaaggcgaagggaaggaggaaggagagggcgaggaagtggaaggcgagagggaaaaggaggaaggagaacggaagaaagaggaaagagccggcaaagaggaaaagggcgaggaagagggcgatcagggcgaaggcgaggaggaagagaccgagggccgcggggaagagaaagaggagggaggagaggtggagggcggagaggtcgaagagggaaagggcgagcgcgaagaggaagaggaagagggcgagggcgaggaagaagagggcgagggggaagaagaggagggagagggcgaagaggaagagggggagggaaagggcgaagaggaaggagaggaaggggagggagaggaagagggggaggagggcgagggggaaggcgaggaggaagaaggagagggggaaggcgaagaggaaggcgagggggaaggagaggaggaagaaggggaaggcgaaggcgaagaggagggagaaggagagggggaggaagaggaaggagaagggaagggcgaggaggaaggcgaagagggagagggggaaggcgaggaagaggaaggcgagggcgaaggagaggacggcgagggcgagggagaagaggaggaaggggaatgggaaggcgaagaagaggaaggcgaaggcgaaggcgaagaagagggcgaaggggagggcgaggagggcgaaggcgaaggggaggaagaggaaggcgaaggagaaggcgaggaagaagagggagaggaggaaggcgaggaggaaggagagggggaggaggagggagaaggcgagggcgaagaagaagaagagggagaagtggagggcgaagtcgagggggaggagggagaaggggaaggggaggaagaagagggcgaagaagaaggcgaggaaagagaaaaagagggagaaggcgaggaaaaccggagaaatagggaagaggaggaagaggaagagggaaagtaccaggagacaggcgaagaggaaaacgagcggcaggatggcgaggaatataagaaagtgagcaagatcaaaggatccgtcaagtacggcaagcacaaaacctatcagaagaaaagcgtgaccaacacacaggggaatggaaaagagcagaggagtaagatgcctgtgcagtcaaaacggctgctgaagaatggcccatctggaagtaaaaaattctggaacaatgtgctgccccactatctggaactgaaataa

SEQ ID NO: 81 - DNA, 3459 bp, Artificial Sequence; identical to SEQ ID NO: 80
  Made in Lab - Codon optimized RPGR ORF15

SEQ ID NO: 82 - DNA, 199 bp, Homo sapiens; identical to SEQ ID NO: 5

SEQ ID NO: 83 - DNA, 269 bp, Bos taurus; identical to SEQ ID NO: 8

SEQ ID NO: 84 - DNA, 664 bp, Artificial Sequence
  Recombinant synthesis - CMV-CBA promoter variant
ctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 85 - DNA, 686 bp, Artificial Sequence
  Recombinant synthesis - CBA-RBG promoter variant
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 86 - DNA, 440 bp, Artificial Sequence
  Recombinant synthesis - CBA-InEx promoter variant
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcatt